(19) United States
(12) Patent Application Publication
(10) Pub. No.: US 2023/0324389 A1
(43) Pub. Date: Oct. 12, 2023

(54) COMPOSITIONS AND METHODS RELATED TO ACTIVATABLE THERAPEUTIC AGENTS

(71) Applicant: AMUNIX PHARMACEUTICALS, INC., Mountain View, CA (US)

(72) Inventors: Volker SCHELLENBERGER, Palo Alto, CA (US); Deena RENNERFELDT, Redwood City, CA (US); Angela HENKENSIEFKEN, San Jose, CA (US); Milton TO, San Leandro, CA (US)

(21) Appl. No.: 18/068,872

(22) Filed: Dec. 20, 2022

Related U.S. Application Data
(63) Continuation of application No. PCT/US2021/042426, filed on Jul. 20, 2021.
(60) Provisional application No. 63/054,525, filed on Jul. 21, 2020.

(51) Int. Cl. G01N 33/573; G01N 33/68

(52) U.S. Cl. CPC G01N 33/573; G01N 2333/8146; G01N 33/6803

(57) ABSTRACT

Described herein are methods for assessing likelihood of response of subjects to activatable therapeutic agents and compositions, kits, and methods of preparing and using activatable therapeutic agents. Also described herein are methods for assessing likelihood of response of subjects to activatable therapeutic agents. In some cases, the activatable therapeutic agents of the compositions, kits, and methods disclosed herein can comprise a mammalian protein-derived sequence.

### RELATED APPLICATIONS

This application is a continuation of International Patent Application No. PCT/US2021/042426, filed Jul. 20, 2021, which claims priority to U.S. Provisional Patent Application Ser. No. 63/054,525 filed Jul. 21, 2020, the entire disclosure of which is hereby incorporated herein by reference.

### SEQUENCE LISTING

The instant application contains a Sequence Listing which has been submitted electronically in XML format and is hereby incorporated by reference in its entirety. Said XML file, created on Dec. 19, 2022, is named 737804_SA9-740PCCON_ST26.xml and is 4,005,294 bytes in size.

### BACKGROUND

A key challenge in developing prodrug therapeutics is avoiding unwanted immunogenicity and nonspecific activation at biological sites in vivo other than the target site. Various release sites have been optimized in vitro and incorporated into prodrugs for programmed and targeted activation, for example, by protease(s) natively produced at or near diseased tissue(s). Such engineered release segments can form T- or B-cell epitopes that can elicit undesired immunogenicity in patients. Further, there is currently a lack of methods for adequately predicting in vivo responses of patients to prodrugs. In particular, with respect to protease-activated prodrugs, diseased tissues being targeted often contain a multitude of proteases with varying activities and specificities, which is difficult to reconstitute in vitro and complicates any prediction of in vivo prodrug activation. There remains a need for identifying new peptide segments that can be incorporated into a variety of prodrug therapeutic, diagnostic and prophylactic compositions for a more effective and reliable release mechanism. There also remains a need for developing more accurate and robust methods for predicting therapeutic responses and outcomes upon administration of prodrugs or other activatable compositions.

### SUMMARY

In certain aspects, the present disclosure provides a method for assessing a likelihood of a subject being responsive to a therapeutic agent that is activatable by a mammalian protease expressed in the subject, the method comprising:
(a) determining, in a biological sample from the subject, a presence or an amount of
   (i) a polypeptide comprising at least five, at least six, at least seven, at least eight, at least nine, or at least ten consecutive amino acid residues shown in a sequence set forth in Column V of Table A (or a subset thereof); or
   (ii) a polypeptide comprising at least five, at least six, at least seven, at least eight, at least nine, or at least ten consecutive amino acids shown in a sequence set forth in Column IV of Table A (or a subset thereof); or
   (iii) a polypeptide comprising at least five, at least six, at least seven, at least eight, at least nine, or at least ten consecutive amino acids shown in a sequence set forth in Column VI of Table A (or a subset thereof); and
(b) designating the subject as being likely to respond to the therapeutic agent when the polypeptide of (i), (ii) or (iii) is present and/or if its amount exceeds a threshold.

In some embodiments of the method for assessing the likelihood of the subject being responsive to the therapeutic agent, the therapeutic agent comprises a peptide substrate, which peptide substrate is susceptible to cleavage by the mammalian protease at a scissile bond. In some embodiments, the polypeptide of (i), (ii), or (iii) comprises a portion containing at least four, at least five, at least six, at least seven, at least eight, at least nine, or at least ten consecutive amino acid residues of the peptide substrate that is either N-terminal or C-terminal side of the scissile bond. In some embodiments, the peptide substrate is susceptible to cleavage by the mammalian protease at a scissile bond, and wherein the polypeptide of (i), (ii), or (iii) is a cleavage product of a reporter polypeptide comprising a substrate sequence that is susceptible to cleavage by the same mammalian protease at a scissile bond and where the reporter polypeptide comprises a sequence set forth in Column II or III of Table A (or a subset thereof). In some embodiments, the peptide substrate is susceptible to cleavage by the mammalian protease at a scissile bond, and wherein the polypeptide of (i), (ii), or (iii) is a cleavage product of a human protein that comprises a portion containing at least five or six consecutive amino acid residues of the peptide substrate that includes the scissile bond.

In some embodiments of the method for assessing the likelihood of the subject being responsive to the therapeutic agent, the polypeptide of (i) comprises at least six, at least seven, at least eight, at least nine, or at least ten consecutive amino acid residues shown in a sequence set forth in Column V of Table A (or a subset thereof). In some embodiments, the polypeptide of (ii) comprises at least six, at least seven, at least eight, at least nine, or at least ten consecutive amino acids shown in a sequence set forth in Column IV of Table A (or a subset thereof). In some embodiments, the polypeptide of (iii) comprises at least six, at least seven, at least eight, at least nine, or at least ten consecutive amino acids shown in a sequence set forth in Column VI of Table A (or a subset thereof).

In some embodiments of the method for assessing the likelihood of the subject being responsive to the therapeutic agent, (a) comprises determining the presence or the amount of any two of (i)-(iii). In some embodiments, (a) comprises determining the presence or the amount of all three of (i)-(iii).

In some embodiments of the method for assessing the likelihood of the subject being responsive to the therapeutic agent, the threshold is zero or nominal. In some embodiments, the biological sample comprises a serum or plasma sample. In some embodiments, the biological sample comprises a serum sample. In some embodiments, the biological sample comprises a plasma sample.

In some embodiments of the method for assessing the likelihood of the subject being responsive to the therapeutic agent, the mammalian protease is a serine protease, a cysteine protease, an aspartate protease, a threonine protease, or a metalloproteinase. In some embodiments, the mammalian protease is selected from the group consisting of disintegrin and metalloproteinase domain-containing protein 10 (ADAM10), disintegrin and metalloproteinase domain-containing protein 12 (ADAM12), disintegrin and metalloproteinase domain-containing protein 15 (ADAM15), disintegrin and metalloproteinase domain-containing protein 17 (ADAM17), disintegrin and metalloproteinase domain-containing protein 9 (ADAM9), disintegrin and metalloproteinase with thrombospondin motifs 5 (ADAMTS5), Cathepsin B, Cathepsin D, Cathepsin E, Cathepsin K, cathepsin L, cathepsin S, Fibroblast activation protein alpha, Hepsin, kallikrein-2, kallikrein-4, kallikrein-3, Prostate-specific antigen (PSA), kallikrein-13, Legumain, matrix metallopeptidase 1 (MMP-1), matrix metallopeptidase 10 (MMP-10), matrix metallopeptidase 11 (MMP-11), matrix metallopeptidase 12 (MMP-12), matrix metallopeptidase 13 (MMP-13), matrix metallopeptidase 14 (MMP-14), matrix metallopeptidase 16 (MMP-16), matrix metallopeptidase 2 (MMP-2), matrix metallopeptidase 3 (MMP-3), matrix metallopeptidase 7 (MMP-7), matrix metallopeptidase 8 (MMP-8), matrix metallopeptidase 9 (MMP-9), matrix metallopeptidase 4 (MMP-4), matrix metallopeptidase 5 (MMP-5), matrix metallopeptidase 6 (MMP-6), matrix metallopeptidase 15 (MMP-15), neutrophil elastase, protease activated receptor 2 (PAR2), plasmin, prostasin, PSMA-FOLH1, membrane type serine protease 1 (MT-SP1), matriptase, and u-plasminogen. In some embodiments, the mammalian protease is selected from the group consisting of matrix metallopeptidase 1 (MMP1), matrix metallopeptidase 2 (MMP2), matrix metallopeptidase 7 (MMP1), matrix metallopeptidase 9 (MMP9), matrix metallopeptidase 11 (MMP11), matrix metallopeptidase 14 (MMP14), urokinase-type plasminogen activator (uPA), legumain, and matriptase. In some embodiments, the mammalian protease is preferentially expressed or activated in a target tissue or cell.

In some embodiments of the method for assessing the likelihood of the subject being responsive to the therapeutic agent, the target tissue or cell is a tumor. In some embodiments, the target tissue or cell produces or is co-localized with the mammalian protease.

In some embodiments of the method for assessing the likelihood of the subject being responsive to the therapeutic agent, the target tissue or cell contains therein or thereon, or is associated with in proximity thereto, a reporter polypeptide. In some embodiments, the reporter polypeptide is a polypeptide selected from the group consisting of coagulation factor, complement component, tubulin, immunoglobulin, apolipoprotein, serum amyloid, insulin, growth factor, fibrinogen, PDZ domain protein, LIM domain protein, c-reactive protein, serum albumin, versican, collagen, elastin, keratin, kininogen-1, alpha-2-antiplasmin, clusterin, biglycan, alpha-1-antitrypsin, transthyretin, alpha-1-antichymotrypsin, glucagon, hepcidin, thymosin beta-4, haptoglobin, hemoglobin subunit alpha, caveolae-associated protein 2, alpha-2-HS-glycoprotein, chromogranin-A, vitronectin, hemopexin, epididymis secretory sperm binding protein, secretogranin-2, angiotensinogen, transgelin-2, pancreatic prohormone, neurosecretory protein VGF, ceruloplasmin, PDZ and LIM domain protein 1, multimerin-1, inter-alpha-trypsin inhibitor heavy chain H2, N-acetylmuramoyl-L-alanine amidase, histone H1.4, adhesion G-protein coupled receptor G6, mannan-binding lectin serine protease 2, prothrombin, deleted in malignant brain tumors 1 protein, desmoglein-3, calsyntenin-1, alpha-2-macroglobulin, myosin-9, sodium/potassium-transporting ATPase subunit gamma, oncoprotein-induced transcript 3 protein, serglycin, histidine-rich glycoprotein, inter-alpha-trypsin inhibitor heavy chain H5, integrin alpha-IIb, membrane-associated progesterone receptor component 1, histone H1.2, rho GDP-dissociation inhibitor 2, zinc-alpha-2-glycoprotein, talin-1, secretogranin-1, neutrophil defensin 3, cytochrome P450 2E1, gastric inhibitory polypeptide, transcription initiation factor TFIID subunit 1, integral membrane protein 2B, pigment epithelium-derived factor, voltage-dependent N-type calcium channel subunit alpha-1B, ras GTPase-activating protein nGAP, type I cytoskeletal 17, sulfhydryl oxidase 1, homeobox protein Hox-B2, transcription factor SOX-10, E3 ubiquitin-protein ligase SIAH2, decorin, secreted protein acidic and rich in cysteine (SPARC), laminin gamma 1 chain, vimentin, and nidogen-1 (NID1). In some embodiments, the reporter polypeptide is a polypeptide selected from the group consisting of versican, type II collagen alpha-1 chain, kininogen-1, complement C4-A, complement C4-B, complement C3, alpha-2-antiplasmin, clusterin, biglycan, elastin, fibrinogen alpha chain, alpha-1-antitrypsin, fibrinogen beta chain, type III collagen alpha-1 chain, serum amyloid A-1 protein, transthyretin, apolipoprotein A-I, apolipoprotein A-I Isoform 1, alpha-1-antichymotrypsin, glucagon, hepcidin, serum amyloid A-2 protein, thymosin beta-4, haptoglobin, hemoglobin subunit alpha, caveolae-associated protein 2, alpha-2-HS-glycoprotein, chromogranin-A, vitronectin, hemopexin, epididymis secretory sperm binding protein, zyxin, apolipoprotein secretogranin-2, angiotensinogen, c-reactive protein, serum albumin, transgelin-2, pancreatic prohormone, neurosecretory protein VGF, ceruloplasmin, PDZ and LIM domain protein 1, tubulin alpha-4A chain, multimerin-1, inter-alpha-trypsin inhibitor heavy chain H2, apolipoprotein C-I, fibrinogen gamma chain, N-acetylmuramoyl-L-alanine amidase, immunoglobulin lambda variable 3-21, histone H1.4, adhesion G-protein coupled receptor G6, immunoglobulin lambda variable 3-25, immunoglobulin lambda variable 1-51, immunoglobulin lambda variable 1-36, mannan-binding lectin serine protease 2, immunoglobulin kappa variable 3-20, immunoglobulin kappa variable 2-30, insulin-like growth factor II, apolipoprotein A-II, probable non-functional immunoglobulin kappa variable 2D-24, prothrombin, coagulation factor IX, apolipoprotein L1, deleted in malignant brain tumors 1 protein, desmoglein-3, calsyntenin-1, immunoglobulin lambda constant 3, complement C5, alpha-2-macroglobulin, myosin-9, sodium/potassium-transporting ATPase subunit gamma, immunoglobulin kappa variable 2-28, oncoprotein-induced transcript 3 protein, serglycin, coagulation factor XII, coagulation factor XIII A chain, insulin, histidine-rich glycoprotein, immunoglobulin kappa variable 3-11, immunoglobulin kappa variable 1-39, collagen alpha-1(I) chain, inter-alpha-trypsin inhibitor heavy chain H5, latent-transforming growth factor beta-binding protein 2, integrin alpha-11b, membrane-associated progesterone receptor component 1, immunoglobulin lambda variable 6-57, immunoglobulin kappa variable 3-15, complement C1r subcomponent-like protein, histone H1.2, rho GDP-dissociation inhibitor 2, latent-transforming growth factor beta-binding protein 4, collagen alpha-1(XVIII) chain, immunoglobulin lambda variable 2-18, zinc-alpha-2-glycoprotein, talin-1, secretogranin-1, neutrophil defensin 3, cytochrome P450 2E1, gastric inhibitory polypeptide, immunoglobulin heavy variable 3-15, immunoglobulin lambda variable 2-11, transcription initiation factor TFIID subunit 1, collagen alpha-1(VII) chain, integral membrane protein 2B, pigment epithelium-derived factor, voltage-dependent N-type calcium channel subunit alpha-1B, immunoglobulin lambda variable 3-27, ras GTPase-activating protein nGAP, keratin, type I cytoskeletal 17, tubulin beta chain, sulfhydryl oxidase 1, immunoglobulin kappa variable 4-1, complement C1r subcomponent, homeobox protein Hox-B2, transcription factor SOX-10, E3 ubiquitin-protein ligase SIAH2, decorin, SPARC, type I collagen alpha-1 chain, type IV collagen alpha-1 chain, laminin gamma 1 chain, vimentin, type III collagen, type IV collagen alpha-3 chain, type VII collagen alpha-1 chain, type VI collagen alpha-1 chain, type V collagen alpha-1 chain, nidogen-1, and type VI collagen alpha-3 chain. In some embodiments, the reporter polypeptide comprises a sequence set forth in Columns II-VI of Table A (or a subset thereof). In some embodiments, the reporter polypeptide is selected from the group set forth in Column I of Table A (or a subset thereof).

In some embodiments of the method for assessing the likelihood of the subject being responsive to the therapeutic agent, the target tissue or cell is characterized by an increased amount or activity of the mammalian protease in proximity to the target tissue or cell as compared to a non-target tissue or cell in the subject. In some embodiments, the subject is suffering from, or is suspected of suffering from, a disease or condition characterized by an increased expression or activity of the mammalian protease in proximity to a target tissue or cell as compared to a corresponding non-target tissue or cell in the subject.

In some embodiments of the method for assessing the likelihood of the subject being responsive to the therapeutic agent, the disease or condition is a cancer or an inflammatory or autoimmune disease. In some embodiments, the disease or condition is selected from the group consisting of carcinoma, Hodgkin's lymphoma, and non-Hodgkin's lymphoma, diffuse large B cell lymphoma, follicular lymphoma, mantle cell lymphoma, blastoma, breast cancer, ER/PR+ breast cancer, Her2+ breast cancer, triple-negative breast cancer, colon cancer, colon cancer with malignant ascites, mucinous tumors, prostate cancer, head and neck cancer, skin cancer, melanoma, genito-urinary tract cancer, ovarian cancer, ovarian cancer with malignant ascites, peritoneal carcinomatosis, uterine serous carcinoma, endometrial cancer, cervix cancer, colorectal, uterine cancer, mesothelioma in the peritoneum, kidney cancer, Wilm's tumor, lung cancer, small-cell lung cancer, non-small cell lung cancer, gastric cancer, stomach cancer, small intestine cancer, liver cancer, hepatocarcinoma, hepatoblastoma, liposarcoma, pancreatic cancer, gall bladder cancer, cancers of the bile duct, esophageal cancer, salivary gland carcinoma, thyroid cancer, epithelial cancer, arrhenoblastoma, adenocarcinoma, sarcoma, and B-cell derived chronic lymphatic leukemia. In some embodiments, the disease or condition is selected from the group consisting of ankylosing spondylitis (AS), arthritis (for example, and not limited to, rheumatoid arthritis (RA), juvenile idiopathic arthritis (JIA), osteoarthritis (OA), psoriatic arthritis (PsA), gout, chronic arthritis), chagas disease, chronic obstructive pulmonary disease (COPD), dermatomyositis, type 1 diabetes, endometriosis, Goodpasture syndrome, Graves' disease, Guillain-Barre syndrome (GB S), Hashimoto's disease, suppurative scab, Kawasaki disease, IgA nephropathy, idiopathic thrombocytopenic purpura, inflammatory bowel disease (IBD) (for example, and not limited to, Crohn's disease (CD), clonal disease, ulcerative colitis, collagen colitis, lymphocytic colitis, ischemic colitis, empty colitis, Behcet's syndrome, infectious colitis, indeterminate colitis, interstitial Cystitis), lupus (for example, and not limited to, systemic lupus erythematosus, discoid lupus, subacute cutaneous lupus erythematosus, cutaneous lupus erythematosus (such as chilblain lupus erythematosus), drug-induced lupus, neonatal lupus, lupus nephritis), mixed connective tissue disease, morphea, multiple sclerosis (MS), severe muscle Force disorder, narcolepsy, neuromuscular angina, pemphigus vulgaris, pernicious anemia, psoriasis, psoriatic arthritis, polymyositis, primary biliary cirrhosis, relapsing polychondritis, schizophrenia, scleroderma, Sjogren's syndrome, systemic stiffness syndrome, temporal arteritis (also known as giant cell arteritis), vasculitis, vitiligo, Wegener's granulomatosis, transplant rejection-associated immune reaction(s) (for example, and not limited to, renal transplant rejection, lung transplant rejection, liver transplant rejection), psoriasis, Wiskott-Aldrich syndrome, autoimmune lymphoproliferative syndrome, myasthenia gravis, inflammatory chronic rhinosinusitis, colitis, celiac disease, Barrett's esophagus, inflammatory gastritis, autoimmune nephritis, autoimmune hepatitis, autoimmune carditis, autoimmune encephalitis, autoimmune mediated hematological disease, asthma, atopic dermatitis, atopy, allergy, allergic rhinitis, scleroderma, bronchitis, pericarditis, the inflammatory disease is, Alzheimer's disease, Parkinson's disease, amyotrophic lateral sclerosis, inflammatory lung disease, inflammatory skin disease, atherosclerosis, myocardial infarction, stroke, gram-positive shock, gram-negative shock, sepsis, septic shock, hemorrhagic shock, anaphylactic shock, systemic inflammatory response syndrome.

In some embodiments of the method for assessing the likelihood of the subject being responsive to the therapeutic agent, the therapeutic agent is an anti-cancer agent. In some embodiments, the therapeutic agent is an activatable therapeutic agent. In some embodiments, the therapeutic agent is an activatable therapeutic agent, or non-natural, activatable therapeutic agent as described herein.

In some embodiments of the method for assessing the likelihood of the subject being responsive to the therapeutic agent, the therapeutic agent further comprises a masking moiety (MM). In some embodiments of the method for assessing the likelihood of the subject being responsive to the therapeutic agent, the masking moiety (MM) is capable of being released from the therapeutic agent upon cleavage of the peptide substrate by the mammalian protease. In some embodiments, the masking moiety (MM) interferes with an interaction of the therapeutic agent, in an uncleaved state, to a target tissue or cell. In some embodiments, a bioactivity of the therapeutic agent is capable of being enhanced upon cleavage of the peptide substrate by the mammalian protease. In some embodiments, the masking moiety (MM) is an extended recombinant polypeptide (XTEN). In some embodiments, the XTEN is characterized in that: (i) it comprises at least 100 amino acids; (ii) at least 90% of the amino acid residues of it are selected from glycine (G), alanine (A), serine (S), threonine (T), glutamate (E) and proline (P); and (iii) it comprises at least 4 different types of amino acids selected from G, A, S, T, E, and P.

In some embodiments of the method for assessing the likelihood of the subject being responsive to the therapeutic agent, further comprises transmitting the designation to a healthcare provider and/or the subject.

In some embodiments of the method for assessing the likelihood of the subject being responsive to the therapeutic agent, further comprises, subsequent to (b), contacting the therapeutic agent with the mammalian protease.

In some embodiments of the method for assessing the likelihood of the subject being responsive to the therapeutic agent, further comprises, subsequent to (b), administering to the subject an effective amount of the therapeutic agent based on the designation of step (b).

In some embodiments of the method for assessing the likelihood of the subject being responsive to the therapeutic agent, (a) comprises detecting the polypeptide of (i), (ii) or (iii) in an immuno-assay. In some embodiments, the immuno-assay utilizes an antibody that specifically binds to the polypeptide of (i), (ii) or (iii), or an epitope thereof.

In some embodiments of the method for assessing the likelihood of the subject being responsive to the therapeutic agent, (a) comprises detecting the polypeptide of (i), (ii) or (iii) (or a derivative (including fragment(s)) thereof) by using a mass spectrometer (MS)

In some embodiment of the method is use of a diagnostic reagent for assessing a likelihood of a subject being responsive to a therapeutic agent that is activatable by a mammalian protease expressed in said subject having a disease or disorder.

In certain aspects the diagnostic reagent is used for assessing a likelihood of a subject being responsive to a therapeutic agent that is activatable by a mammalian protease expressed in said subject having a disease or disorder.

In some embodiments is a kit for the practice of a method for assessing a likelihood of a subject being responsive to a therapeutic agent that is activatable by a mammalian protease expressed in said subject having a disease or disorder comprising a reagent for detecting the presence or amount of a proteolytic peptide product produced by action of said mammalian protease.

In certain aspects, the present disclosure provides a method for treating a subject in need of a therapeutic agent that is activatable by a mammalian protease expressed in the subject, the method comprising: administering an effective amount of the therapeutic agent to the subject, wherein the subject has been shown to express in a biological sample from the subject:
(i) a polypeptide comprising at least five, at least six, at least seven, at least eight, at least nine, or at least ten consecutive amino acid residues shown in a sequence set forth in Column V of Table A (or a subset thereof); or
(ii) a polypeptide comprising at least five, at least six, at least seven, at least eight, at least nine, or at least ten consecutive amino acids shown in a sequence set forth in Column IV of Table A (or a subset thereof); or
(iii) a polypeptide comprising at least five, at least six, at least seven, at least eight, at least nine, or at least ten consecutive amino acids shown in a sequence set forth in Column VI of Table A (or a subset thereof); or
(iv) expression level of polypeptide (i), (ii) or (iii) exceeds a threshold.

In some embodiments for treating the subject with the therapeutic agent, the polypeptide of (i) comprises at least six, at least seven, at least eight, at least nine, or at least ten consecutive amino acid residues shown in a sequence set forth in Column V of Table A (or a subset thereof). In some embodiments, the polypeptide of (ii) comprises at least six, at least seven, at least eight, at least nine, or at least ten consecutive amino acids shown in a sequence set forth in Column IV of Table A (or a subset thereof). In some embodiments, the polypeptide of (iii) comprises at least six, at least seven, at least eight, at least nine, or at least ten consecutive amino acids shown in a sequence set forth in Column VI of Table A (or a subset thereof). In some embodiments, the subject has been shown to express in the biological sample any two of (i)-(iii). In some embodiments, the subject has been shown to express in the biological sample all three of (i)-(iii).

In some embodiments for treating the subject with the therapeutic agent, the therapeutic agent comprises a peptide substrate susceptible to cleavage by the mammalian protease. In some embodiments, the peptide substrate is susceptible to cleavage by the mammalian protease at a scissile bond, and wherein the polypeptide of (i), (ii), or (iii) comprises a portion containing at least four consecutive amino acid residues of the peptide substrate that is either N-terminal or C-terminal of the scissile bond. In some embodiments, a portion of the peptide substrate that is N-terminal of the scissile bond has at most three or two amino acid substitutions or at most one amino acid substitution with respect to a C-terminal end sequence containing from four to ten amino acid residues of a sequence set forth in Column IV or V of Table A (or a subset thereof), wherein none of the amino acid substitution is at a position corresponding to an amino acid residue immediately adjacent to a corresponding scissile bond. In some embodiments, a portion of the peptide substrate that is N-terminal of the scissile bond has at most three or two amino acid substitutions or at most one amino acid substitution with respect to a C-terminal end sequence containing from four to ten amino acid residues of a sequence set forth in Column IV of Table A (or a subset thereof), wherein none of the amino acid substitution is at a position corresponding to an amino acid residue immediately adjacent to a corresponding scissile bond. In some embodiments, a portion of the peptide substrate that is N-terminal of the scissile bond has at most three or two amino acid substitutions or at most one amino acid substitution with respect to a C-terminal end sequence containing from four to ten amino acid residues of a sequence set forth in Column V of Table A (or a subset thereof), wherein none of the amino acid substitution is at a position corresponding to an amino acid residue immediately adjacent to a corresponding scissile bond. In some embodiments, the portion of the peptide substrate that is N-terminal of the scissile bond comprises a C-terminal end sequence containing from four to ten amino acid residues of a sequence set forth in Column IV or V of Table A (or a subset thereof). In some embodiments, the portion of the peptide substrate that is N-terminal of the scissile bond comprises a C-terminal end sequence containing from four to ten amino acid residues of a sequence set forth in Column IV of Table A (or a subset thereof). In some embodiments, the portion of the peptide substrate that is N-terminal of the scissile bond comprises a C-terminal end sequence containing from four to ten amino acid residues of a sequence set forth in Column V of Table A (or a subset thereof). In some embodiments, a portion of the peptide substrate that is C-terminal of the scissile bond has at most three or two amino acid substitutions or at most one amino acid substitution with respect to an N-terminal end sequence containing from four to ten amino acid residues of a sequence set forth in Column V or VI of Table A (or a subset thereof), wherein none of the amino acid substitution is at a position corresponding to an amino acid residue immediately adjacent to a corresponding scissile bond. In some embodiments, a portion of the peptide substrate that is C-terminal of the scissile bond has at most three or two amino acid substitutions or at most one amino acid substitution with respect to an N-terminal end sequence containing from four to ten amino acid residues of a sequence set forth in Column V of Table A (or a subset thereof), wherein none of the amino acid substitution is at a position corresponding to an amino acid residue immediately adjacent to a corresponding scissile bond. In some embodiments, a portion of the peptide substrate that is C-terminal of the scissile bond has at most three or two amino acid substitutions or at most one amino acid substitution with respect to an N-terminal end sequence containing from four to ten amino acid residues of a sequence set forth in Column VI of Table A (or a subset thereof), wherein none of the amino acid substitution is at a position corresponding to an amino acid residue immediately adjacent to a corresponding scissile bond. In some embodiments, the portion of the peptide substrate that is C-terminal of the scissile bond comprises an N-terminal end sequence containing from four to ten amino acid residues of a sequence set forth in Column V or VI of Table A (or a subset thereof). In some embodiments, the portion of the peptide substrate that is C-terminal of the scissile bond comprises an N-terminal end sequence containing from four to ten amino acid residues of a sequence set forth in Column V of Table A (or a subset thereof). In some embodiments, the portion of the peptide substrate that is C-terminal of the scissile bond comprises an N-terminal end sequence containing from four to ten amino acid residues of a sequence set forth in Column VI of Table A (or a subset thereof).

In some embodiments for treating the subject with the therapeutic agent, the threshold is zero or nominal. In some embodiments, the biological sample comprises a serum or plasma sample. In some embodiments, the biological sample comprises a serum sample. In some embodiments, the biological sample comprises a plasma sample.

In some embodiments for treating the subject with the therapeutic agent, the mammalian protease is a serine protease, a cysteine protease, an aspartate protease, a threonine protease, or a metalloproteinase. In some embodiments, the mammalian protease is selected from the group consisting of disintegrin and metalloproteinase domain-containing protein 10 (ADAM10), disintegrin and metalloproteinase domain-containing protein 12 (ADAM12), disintegrin and metalloproteinase domain-containing protein 15 (ADAM15), disintegrin and metalloproteinase domain-containing protein 17 (ADAM17), disintegrin and metalloproteinase domain-containing protein 9 (ADAM9), disintegrin and metalloproteinase with thrombospondin motifs 5 (ADAMTS5), Cathepsin B, Cathepsin D, Cathepsin E, Cathepsin K, cathepsin L, cathepsin S, Fibroblast activation protein alpha, Hepsin, kallikrein-2, kallikrein-4, kallikrein-3, Prostate-specific antigen (PSA), kallikrein-13, Legumain, matrix metallopeptidase 1 (MMP-1), matrix metallopeptidase 10 (MMP-10), matrix metallopeptidase 11 (MMP-11), matrix metallopeptidase 12 (MMP-12), matrix metallopeptidase 13 (MMP-13), matrix metallopeptidase 14 (MMP-14), matrix metallopeptidase 16 (MMP-16), matrix metallopeptidase 2 (MMP-2), matrix metallopeptidase 3 (MMP-3), matrix metallopeptidase 7 (MMP-7), matrix metallopeptidase 8 (MMP-8), matrix metallopeptidase 9 (MMP-9), matrix metallopeptidase 4 (MMP-4), matrix metallopeptidase 5 (MMP-5), matrix metallopeptidase 6 (MMP-6), matrix metallopeptidase 15 (MMP-15), neutrophil elastase, protease activated receptor 2 (PAR2), plasmin, prostasin, PSMA-FOLH1, membrane type serine protease 1 (MT-SP1), matriptase, and u-plasminogen. In some embodiments, the mammalian protease is selected from the group consisting of matrix metallopeptidase 1 (MMP1), matrix metallopeptidase 2 (MMP2), matrix metallopeptidase 7 (MMP1), matrix metallopeptidase 9 (MMP9), matrix metallopeptidase 11 (MMP11), matrix metallopeptidase 14 (MMP14), urokinase-type plasminogen activator (uPA), legumain, and matriptase. In some embodiments, the mammalian protease is preferentially expressed or activated in a target tissue or cell. In some embodiments, the target tissue or cell is a tumor. In some embodiments, the target tissue or cell produces or is co-localized with the mammalian protease.

In some embodiments for treating the subject with the therapeutic agent, the target tissue or cell contains therein or thereon, or is associated with in proximity thereto, a reporter polypeptide. In some embodiments, the reporter polypeptide is a polypeptide selected from the group consisting of coagulation factor, complement component, tubulin, immunoglobulin, apolipoprotein, serum amyloid, insulin, growth factor, fibrinogen, PDZ domain protein, LIM domain protein, c-reactive protein, serum albumin, versican, collagen, elastin, keratin, kininogen-1, alpha-2-antiplasmin, clusterin, biglycan, alpha-1-antitrypsin, transthyretin, alpha-1-antichymotrypsin, glucagon, hepcidin, thymosin beta-4, haptoglobin, hemoglobin subunit alpha, caveolae-associated protein 2, alpha-2-HS-glycoprotein, chromogranin-A, vitronectin, hemopexin, epididymis secretory sperm binding protein, secretogranin-2, angiotensinogen, transgelin-2, pancreatic prohormone, neurosecretory protein VGF, ceruloplasmin, PDZ and LIM domain protein 1, multimerin-1, inter-alpha-trypsin inhibitor heavy chain H2, N-acetylmuramoyl-L-alanine amidase, histone H1.4, adhesion G-protein coupled receptor G6, mannan-binding lectin serine protease 2, prothrombin, deleted in malignant brain tumors 1 protein, desmoglein-3, calsyntenin-1, alpha-2-macroglobulin, myosin-9, sodium/potassium-transporting ATPase subunit gamma, oncoprotein-induced transcript 3 protein, serglycin, histidine-rich glycoprotein, inter-alpha-trypsin inhibitor heavy chain H5, integrin alpha-IIb, membrane-associated progesterone receptor component 1, histone H1.2, rho GDP-dissociation inhibitor 2, zinc-alpha-2-glycoprotein, talin-1, secretogranin-1, neutrophil defensin 3, cytochrome P450 2E1, gastric inhibitory polypeptide, transcription initiation factor TFIID subunit 1, integral membrane protein 2B, pigment epithelium-derived factor, voltage-dependent N-type calcium channel subunit alpha-1B, ras GTPase-activating protein nGAP, type I cytoskeletal 17, sulfhydryl oxidase 1, homeobox protein Hox-B2, transcription factor SOX-10, E3 ubiquitin-protein ligase SIAH2, decorin, secreted protein acidic and rich in cysteine (SPARC), laminin gamma 1 chain, vimentin, and nidogen-1 (NID1). In some embodiments, the reporter polypeptide is a polypeptide selected from the group consisting of versican, type II collagen alpha-1 chain, kininogen-1, complement C4-A, complement C4-B, complement C3, alpha-2-antiplasmin, clusterin, biglycan, elastin, fibrinogen alpha chain, alpha-1-antitrypsin, fibrinogen beta chain, type III collagen alpha-1 chain, serum amyloid A-1 protein, transthyretin, apolipoprotein A-I, apolipoprotein A-I Isoform 1, alpha-1-antichymotrypsin, glucagon, hepcidin, serum amyloid A-2 protein, thymosin beta-4, haptoglobin, hemoglobin subunit alpha, caveolae-associated protein 2, alpha-2-HS-glycoprotein, chromogranin-A, vitronectin, hemopexin, epididymis secretory sperm binding protein, zyxin, apolipoprotein secretogranin-2, angiotensinogen, c-reactive protein, serum albumin, transgelin-2, pancreatic prohormone, neurosecretory protein VGF, ceruloplasmin, PDZ and LIM domain protein 1, tubulin alpha-4A chain, multimerin-1, inter-alpha-trypsin inhibitor heavy chain H2, apolipoprotein C-I, fibrinogen gamma chain, N-acetylmuramoyl-L-alanine amidase, immunoglobulin lambda variable 3-21, histone H1.4, adhesion G-protein coupled receptor G6, immunoglobulin lambda variable 3-25, immunoglobulin lambda variable 1-51, immunoglobulin lambda variable 1-36, mannan-binding lectin serine protease 2, immunoglobulin kappa variable 3-20, immunoglobulin kappa variable 2-30, insulin-like growth factor II, apolipoprotein A-II, probable non-functional immunoglobulin kappa variable 2D-24, prothrombin, coagulation factor IX, apolipoprotein L1, deleted in malignant brain tumors 1 protein, desmoglein-3, calsyntenin-1, immunoglobulin lambda constant 3, complement C5, alpha-2-macroglobulin, myosin-9, sodium/potassium-transporting ATPase subunit gamma, immunoglobulin kappa variable 2-28, oncoprotein-induced transcript 3 protein, serglycin, coagulation factor XII, coagulation factor XIII A chain, insulin, histidine-rich glycoprotein, immunoglobulin kappa variable 3-11, immunoglobulin kappa variable 1-39, collagen alpha-1(I) chain, inter-alpha-trypsin inhibitor heavy chain H5, latent-transforming growth factor beta-binding protein 2, integrin alpha-IIb, membrane-associated progesterone receptor component 1, immunoglobulin lambda variable 6-57, immunoglobulin kappa variable 3-15, complement C1r subcomponent-like protein, histone H1.2, rho GDP-dissociation inhibitor 2, latent-transforming growth factor beta-binding protein 4, collagen alpha-1(XVIII) chain, immunoglobulin lambda variable 2-18, zinc-alpha-2-glycoprotein, talin-1, secretogranin-1, neutrophil defensin 3, cytochrome P450 2E1, gastric inhibitory polypeptide, immunoglobulin heavy variable 3-15, immunoglobulin lambda variable 2-11, transcription initiation factor TFIID subunit 1, collagen alpha-1(VII) chain, integral membrane protein 2B, pigment epithelium-derived factor, voltage-dependent N-type calcium channel subunit alpha-1B, immunoglobulin lambda variable 3-27, ras GTPase-activating protein nGAP, keratin, type I cytoskeletal 17, tubulin beta chain, sulfhydryl oxidase 1, immunoglobulin kappa variable 4-1, complement C1r subcomponent, homeobox protein Hox-B2, transcription factor SOX-10, E3 ubiquitin-protein ligase SIAH2, decorin, SPARC, type I collagen alpha-1 chain, type IV collagen alpha-1 chain, laminin gamma 1 chain, vimentin, type III collagen, type IV collagen alpha-3 chain, type VII collagen alpha-1 chain, type VI collagen alpha-1 chain, type V collagen alpha-1 chain, nidogen-1, and type VI collagen alpha-3 chain. In some embodiments, the reporter polypeptide comprises a sequence set forth in Columns II-VI of Table A (or a subset thereof). In some embodiments, the reporter polypeptide is selected from the group set forth in Column I of Table A (or a subset thereof).

In some embodiments for treating the subject with the therapeutic agent, the target tissue or cell is characterized by an increased amount or activity of the mammalian protease in proximity to the target tissue or cell as compared to a non-target tissue or cell in the subject. In some embodiments, the subject is suffering from, or is suspected of suffering from, a disease or condition characterized by an increased expression or activity of the mammalian protease in proximity to a target tissue or cell as compared to a corresponding non-target tissue or cell in the subject. In some embodiments, the disease or condition is a cancer or an inflammatory or autoimmune disease. In some embodiments, the disease or condition is selected from the group consisting of ankylosing spondylitis (AS), arthritis (for example, and not limited to, rheumatoid arthritis (RA), juvenile idiopathic arthritis (JIA), osteoarthritis (OA), psoriatic arthritis (PsA), gout, chronic arthritis), chagas disease, chronic obstructive pulmonary disease (COPD), dermatomyositis, type 1 diabetes, endometriosis, Goodpasture syndrome, Graves' disease, Guillain-Barre syndrome (GBS), Hashimoto's disease, suppurative scab, Kawasaki disease, IgA nephropathy, idiopathic thrombocytopenic purpura, inflammatory bowel disease (IBD) (for example, and not limited to, Crohn's disease (CD), clonal disease, ulcerative colitis, collagen colitis, lymphocytic colitis, ischemic colitis, empty colitis, Behcet's syndrome, infectious colitis, indeterminate colitis, interstitial Cystitis), lupus (for example, and not limited to, systemic lupus erythematosus, discoid lupus, subacute cutaneous lupus erythematosus, cutaneous lupus erythematosus (such as chilblain lupus erythematosus), drug-induced lupus, neonatal lupus, lupus nephritis), mixed connective tissue disease, morphea, multiple sclerosis (MS), severe muscle Force disorder, narcolepsy, neuromuscular angina, pemphigus vulgaris, pernicious anemia, psoriasis, psoriatic arthritis, polymyositis, primary biliary cirrhosis, relapsing polychondritis, schizophrenia, scleroderma, Sjogren's syndrome, systemic stiffness syndrome, temporal arteritis (also known as giant cell arteritis), vasculitis, vitiligo, Wegener's granulomatosis, transplant rejection-associated immune reaction(s) (for example, and not limited to, renal transplant rejection, lung transplant rejection, liver transplant rejection), psoriasis, Wiskott-Aldrich syndrome, autoimmune lymphoproliferative syndrome, myasthenia gravis, inflammatory chronic rhinosinusitis, colitis, celiac disease, Barrett's esophagus, inflammatory gastritis, autoimmune nephritis, autoimmune hepatitis, autoimmune carditis, autoimmune encephalitis, autoimmune mediated hematological disease, asthma, atopic dermatitis, atopy, allergy, allergic rhinitis, scleroderma, bronchitis, pericarditis, the inflammatory disease is, Alzheimer's disease, Parkinson's disease, amyotrophic lateral sclerosis, inflammatory lung disease, inflammatory skin disease, atherosclerosis, myocardial infarction, stroke, gram-positive shock, gram-negative shock, sepsis, septic shock, hemorrhagic shock, anaphylactic shock, systemic inflammatory response syndrome. In some embodiments, the disease or condition is selected from the group consisting of carcinoma, Hodgkin's lymphoma, and non-Hodgkin's lymphoma, diffuse large B cell lymphoma, follicular lymphoma, mantle cell lymphoma, blastoma, breast cancer, ER/PR+ breast cancer, Her2+ breast cancer, triple-negative breast cancer, colon cancer, colon cancer with malignant ascites, mucinous tumors, prostate cancer, head and neck cancer, skin cancer, melanoma, genito-urinary tract cancer, ovarian cancer, ovarian cancer with malignant ascites, peritoneal carcinomatosis, uterine serous carcinoma, endometrial cancer, cervix cancer, colorectal, uterine cancer, mesothelioma in the peritoneum, kidney cancer, Wilm's tumor, lung cancer, small-cell lung cancer, non-small cell lung cancer, gastric cancer, stomach cancer, small intestine cancer, liver cancer, hepatocarcinoma, hepatoblastoma, liposarcoma, pancreatic cancer, gall bladder cancer, cancers of the bile duct, esophageal cancer, salivary gland carcinoma, thyroid cancer, epithelial cancer, arrhenoblastoma, adenocarcinoma, sarcoma, and B-cell derived chronic lymphatic leukemia. In some embodiments, the therapeutic agent is an anti-cancer agent. In some embodiments, the therapeutic agent is an activatable therapeutic agent. In some embodiments, the therapeutic agent is a non-natural, activatable therapeutic agent as described herein.

In some embodiments for treating the subject with the therapeutic agent, the therapeutic agent comprises a masking moiety (MM). In some embodiments, the masking moiety (MM) is capable of being released from the therapeutic agent upon cleavage of the peptide substrate by the mammalian protease. In some embodiments, the masking moiety (MM) interferes with an interaction of the therapeutic agent, in an uncleaved state, to a target tissue or cell. In some embodiments, a bioactivity of the therapeutic agent is capable of being enhanced upon cleavage of the peptide substrate by the mammalian protease. In some embodiments, the masking moiety (MM) is an extended recombinant polypeptide (XTEN). In some embodiments, the XTEN is characterized in that: (i) it comprises at least 100 amino acids; (ii) at least 90% of the amino acid residues of it are selected from glycine (G), alanine (A), serine (S), threonine (T), glutamate (E) and proline (P); and (iii) it comprises at least 4 different types of amino acids selected from G, A, S, T, E, and P.

In some embodiments for treating the subject with the therapeutic agent, the subject is determined to have a likelihood of a response to the therapeutic agent by a method as described herein.

In certain aspects, the present disclosure provides a method for treating a disease or condition in a subject, comprising administering to the subject in need thereof one or more therapeutically effective doses of a therapeutic agent as described herein, or a pharmaceutical composition as described herein.

In some embodiments for the method for treating the disease or condition in the subject, the subject is selected from the group consisting of mouse, rat, monkey, and human. In some embodiments, the subject is a human. In some embodiments, the subject is determined to have a likelihood of a response to the therapeutic agent or the pharmaceutical composition. In some embodiments, the likelihood of the response is 50% or higher. In some embodiments, the likelihood of the response is determined by a method as described herein.

In some embodiments for the method for treating the disease or condition in the subject, the disease or condition is a cancer or an inflammatory or autoimmune disease. In some embodiments, the disease or condition is selected from the group consisting of ankylosing spondylitis (AS), arthritis (for example, and not limited to, rheumatoid arthritis (RA), juvenile idiopathic arthritis (JIA), osteoarthritis (OA), psoriatic arthritis (PsA), gout, chronic arthritis), chagas disease, chronic obstructive pulmonary disease (COPD), dermatomyositis, type 1 diabetes, endometriosis, Goodpasture syndrome, Graves' disease, Guillain-Barre syndrome (GBS), Hashimoto's disease, suppurative scab, Kawasaki disease, IgA nephropathy, idiopathic thrombocytopenic purpura, inflammatory bowel disease (IBD) (for example, and not limited to, Crohn's disease (CD), clonal disease, ulcerative colitis, collagen colitis, lymphocytic colitis, ischemic colitis, empty colitis, Behcet's syndrome, infectious colitis, indeterminate colitis, interstitial Cystitis), lupus (for example, and not limited to, systemic lupus erythematosus, discoid lupus, subacute cutaneous lupus erythematosus, cutaneous lupus erythematosus (such as chilblain lupus erythematosus), drug-induced lupus, neonatal lupus, lupus nephritis), mixed connective tissue disease, morphea, multiple sclerosis (MS), severe muscle Force disorder, narcolepsy, neuromuscular angina, pemphigus vulgaris, pernicious anemia, psoriasis, psoriatic arthritis, polymyositis, primary biliary cirrhosis, relapsing polychondritis, schizophrenia, scleroderma, Sjogren's syndrome, systemic stiffness syndrome, temporal arteritis (also known as giant cell arteritis), vasculitis, vitiligo, Wegener's granulomatosis, transplant rejection-associated immune reaction(s) (for example, and not limited to, renal transplant rejection, lung transplant rejection, liver transplant rejection), psoriasis, Wiskott-Aldrich syndrome, autoimmune lymphoproliferative syndrome, myasthenia gravis, inflammatory chronic rhinosinusitis, colitis, celiac disease, Barrett's esophagus, inflammatory gastritis, autoimmune nephritis, autoimmune hepatitis, autoimmune carditis, autoimmune encephalitis, autoimmune mediated hematological disease, asthma, atopic dermatitis, atopy, allergy, allergic rhinitis, scleroderma, bronchitis, pericarditis, the inflammatory disease is, Alzheimer's disease, Parkinson's disease, amyotrophic lateral sclerosis, inflammatory lung disease, inflammatory skin disease, atherosclerosis, myocardial infarction, stroke, gram-positive shock, gram-negative shock, sepsis, septic shock, hemorrhagic shock, anaphylactic shock, systemic inflammatory response syndrome. In some embodiments, the disease or condition is selected from the group consisting of carcinoma, Hodgkin's lymphoma, and non-Hodgkin's lymphoma, diffuse large B cell lymphoma, follicular lymphoma, mantle cell lymphoma, blastoma, breast cancer, ER/PR+ breast cancer, Her2+ breast cancer, triple-negative breast cancer, colon cancer, colon cancer with malignant ascites, mucinous tumors, prostate cancer, head and neck cancer, skin cancer, melanoma, genito-urinary tract cancer, ovarian cancer, ovarian cancer with malignant ascites, peritoneal carcinomatosis, uterine serous carcinoma, endometrial cancer, cervix cancer, colorectal, uterine cancer, mesothelioma in the peritoneum, kidney cancer, Wilm's tumor, lung cancer, small-cell lung cancer, non-small cell lung cancer, gastric cancer, stomach cancer, small intestine cancer, liver cancer, hepatocarcinoma, hepatoblastoma, liposarcoma, pancreatic cancer, gall bladder cancer, cancers of the bile duct, esophageal cancer, salivary gland carcinoma, thyroid cancer, epithelial cancer, arrhenoblastoma, adenocarcinoma, sarcoma, and B-cell derived chronic lymphatic leukemia.

In certain aspects, the present disclosure provides use of a therapeutic agent as described herein in the preparation of a medicament for the treatment of a disease or condition in a subject.

In certain aspects, the present disclosure provides use of a pharmaceutical composition as described herein in the preparation of a medicament for the treatment of a disease or condition in a subject.

In some embodiments of the use, the subject is selected from the group consisting of mouse, rat, monkey, and human. In some embodiments, the subject is a human. In some embodiments, the subject is determined to have a likelihood of a response to the therapeutic agent or the pharmaceutical composition. In some embodiments, the likelihood of the response is 50% or higher. In some embodiments, the likelihood of the response is determined by a method as described herein.

In some embodiments of the use, the disease or condition is a cancer or an inflammatory or autoimmune disease. In some embodiments, the disease or condition is selected from the group consisting of carcinoma, Hodgkin's lymphoma, and non-Hodgkin's lymphoma, diffuse large B cell lymphoma, follicular lymphoma, mantle cell lymphoma, blastoma, breast cancer, ER/PR+ breast cancer, Her2+ breast cancer, triple-negative breast cancer, colon cancer, colon cancer with malignant ascites, mucinous tumors, prostate cancer, head and neck cancer, skin cancer, melanoma, genito-urinary tract cancer, ovarian cancer, ovarian cancer with malignant ascites, peritoneal carcinomatosis, uterine serous carcinoma, endometrial cancer, cervix cancer, colorectal, uterine cancer, mesothelioma in the peritoneum, kidney cancer, Wilm's tumor, lung cancer, small-cell lung cancer, non-small cell lung cancer, gastric cancer, stomach cancer, small intestine cancer, liver cancer, hepatocarcinoma, hepatoblastoma, liposarcoma, pancreatic cancer, gall bladder cancer, cancers of the bile duct, esophageal cancer, salivary gland carcinoma, thyroid cancer, epithelial cancer, arrhenoblastoma, adenocarcinoma, sarcoma, and B-cell derived chronic lymphatic leukemia. In some embodiments, the disease or condition is selected from the group consisting of ankylosing spondylitis (AS), arthritis (for example, and not limited to, rheumatoid arthritis (RA), juvenile idiopathic arthritis (JIA), osteoarthritis (OA), psoriatic arthritis (PsA), gout, chronic arthritis), chagas disease, chronic obstructive pulmonary disease (COPD), dermatomyositis, type 1 diabetes, endometriosis, Goodpasture syndrome, Graves' disease, Guillain-Barre syndrome (GB S), Hashimoto's disease, suppurative scab, Kawasaki disease, IgA nephropathy, idiopathic thrombocytopenic purpura, inflammatory bowel disease (IBD) (for example, and not limited to, Crohn's disease (CD), clonal disease, ulcerative colitis, collagen colitis, lymphocytic colitis, ischemic colitis, empty colitis, Behcet's syndrome, infectious colitis, indeterminate colitis, interstitial Cystitis), lupus (for example, and not limited to, systemic lupus erythematosus, discoid lupus, subacute cutaneous lupus erythematosus, cutaneous lupus erythematosus (such as chilblain lupus erythematosus), drug-induced lupus, neonatal lupus, lupus nephritis), mixed connective tissue disease, morphea, multiple sclerosis (MS), severe muscle Force disorder, narcolepsy, neuromuscular angina, pemphigus vulgaris, pernicious anemia, psoriasis, psoriatic arthritis, polymyositis, primary biliary cirrhosis, relapsing polychondritis, schizophrenia, scleroderma, Sjogren's syndrome, systemic stiffness syndrome, temporal arteritis (also known as giant cell arteritis), vasculitis, vitiligo, Wegener's granulomatosis, transplant rejection-associated immune reaction(s) (for example, and not limited to, renal transplant rejection, lung transplant rejection, liver transplant rejection), psoriasis, Wiskott-Aldrich syndrome, autoimmune lymphoproliferative syndrome, myasthenia gravis, inflammatory chronic rhinosinusitis, colitis, celiac disease, Barrett's esophagus, inflammatory gastritis, autoimmune nephritis, autoimmune hepatitis, autoimmune carditis, autoimmune encephalitis, autoimmune mediated hematological disease, asthma, atopic dermatitis, atopy, allergy, allergic rhinitis, scleroderma, bronchitis, pericarditis, the inflammatory disease is, Alzheimer's disease, Parkinson's disease, amyotrophic lateral sclerosis, inflammatory lung disease, inflammatory skin disease, atherosclerosis, myocardial infarction, stroke, gram-positive shock, gram-negative shock, sepsis, septic shock, hemorrhagic shock, anaphylactic shock, systemic inflammatory response syndrome.

In some aspects, the present disclosure provides a therapeutic agent (e.g., activatable therapeutic agent, or non-natural, activatable therapeutic agent) comprising a release segment (RS) linked, directly or indirectly, to a biologically active moiety (BM), wherein the RS comprises a peptide substrate having an amino acid sequence susceptible to cleavage by a mammalian protease at a scissile bond, wherein the peptide substrate comprises an amino acid sequence having at most three amino acid substitutions (or at most two amino acid substitutions, or at most one amino acid substitution) with respect to a sequence set forth in Column II or III of Table A (or a subset thereof).

In some aspects, the present disclosure provides a therapeutic agent (e.g., activatable therapeutic agent, or non-natural, activatable therapeutic agent) comprising a release segment (RS) linked, directly or indirectly, to a biologically active moiety (BM), wherein the RS comprises a peptide substrate having an amino acid sequence susceptible to cleavage by a mammalian protease at a scissile bond, wherein the therapeutic agent is configured for activation at or in proximity to a target tissue or cell in a subject,

wherein the target tissue or cell contains therein or thereon, or is associated with in proximity thereto, a reporter sequence capable of being cleaved by the mammalian protease at a cleavage sequence, and

wherein the peptide substrate comprises an amino acid sequence having at most three amino acid substitutions (or at most two amino acid substitutions, or at most one amino acid substitution) with respect to the cleavage sequence of the reporter polypeptide.

In some embodiments of the therapeutic agent, the reporter polypeptide is a coagulation factor, complement component, tubulin, immunoglobulin, apolipoprotein, serum amyloid, insulin, growth factor, fibrinogen, PDZ domain protein, LIM domain protein, c-reactive protein, serum albumin, versican, collagen, elastin, keratin, kininogen-1, alpha-2-antiplasmin, clusterin, biglycan, alpha-1-antitrypsin, transthyretin, alpha-1-antichymotrypsin, glucagon, hepcidin, thymosin beta-4, haptoglobin, hemoglobin subunit alpha, caveolae-associated protein 2, alpha-2-HS-glycoprotein, chromogranin-A, vitronectin, hemopexin, epididymis secretory sperm binding protein, secretogranin-2, angiotensinogen, transgelin-2, pancreatic prohormone, neurosecretory protein VGF, ceruloplasmin, PDZ and LIM domain protein 1, multimerin-1, inter-alpha-trypsin inhibitor heavy chain H2, N-acetylmuramoyl-L-alanine amidase, histone H1.4, adhesion G-protein coupled receptor G6, mannan-binding lectin serine protease 2, prothrombin, deleted in malignant brain tumors 1 protein, desmoglein-3, calsyntenin-1, alpha-2-macroglobulin, myosin-9, sodium/potassium-transporting ATPase subunit gamma, oncoprotein-induced transcript 3 protein, serglycin, histidine-rich glycoprotein, inter-alpha-trypsin inhibitor heavy chain H5, integrin alpha-IIb, membrane-associated progesterone receptor component 1, histone H1.2, rho GDP-dissociation inhibitor 2, zinc-alpha-2-glycoprotein, talin-1, secretogranin-1, neutrophil defensin 3, cytochrome P450 2E1, gastric inhibitory polypeptide, transcription initiation factor TFIID subunit 1, integral membrane protein 2B, pigment epithelium-derived factor, voltage-dependent N-type calcium channel subunit alpha-1B, ras GTPase-activating protein nGAP, type I cytoskeletal 17, sulfhydryl oxidase 1, homeobox protein Hox-B2, transcription factor SOX-10, E3 ubiquitin-protein ligase SIAH2, decorin, secreted protein acidic and rich in cysteine (SPARC), laminin gamma 1 chain, vimentin, and nidogen-1 (NID1).

In some embodiments of the therapeutic agent, the reporter polypeptide is a polypeptide selected from the group consisting of versican, type II collagen alpha-1 chain, kininogen-1, complement C4-A, complement C4-B, complement C3, alpha-2-antiplasmin, clusterin, biglycan, elastin, fibrinogen alpha chain, alpha-1-antitrypsin, fibrinogen beta chain, type III collagen alpha-1 chain, serum amyloid A-1 protein, transthyretin, apolipoprotein A-I, apolipoprotein A-I Isoform 1, alpha-1-antichymotrypsin, glucagon, hepcidin, serum amyloid A-2 protein, thymosin beta-4, haptoglobin, hemoglobin subunit alpha, caveolae-associated protein 2, alpha-2-HS-glycoprotein, chromogranin-A, vitronectin, hemopexin, epididymis secretory sperm binding protein, zyxin, apolipoprotein secretogranin-2, angiotensinogen, c-reactive protein, serum albumin, transgelin-2, pancreatic prohormone, neurosecretory protein VGF, ceruloplasmin, PDZ and LIM domain protein 1, tubulin alpha-4A chain, multimerin-1, inter-alpha-trypsin inhibitor heavy chain H2, apolipoprotein C-I, fibrinogen gamma chain, N-acetylmuramoyl-L-alanine amidase, immunoglobulin lambda variable 3-21, histone H1.4, adhesion G-protein coupled receptor G6, immunoglobulin lambda variable 3-25, immunoglobulin lambda variable 1-51, immunoglobulin lambda variable 1-36, mannan-binding lectin serine protease 2, immunoglobulin kappa variable 3-20, immunoglobulin kappa variable 2-30, insulin-like growth factor II, apolipoprotein A-II, probable non-functional immunoglobulin kappa variable 2D-24, prothrombin, coagulation factor IX, apolipoprotein L1, deleted in malignant brain tumors 1 protein, desmoglein-3, calsyntenin-1, immunoglobulin lambda constant 3, complement C5, alpha-2-macroglobulin, myosin-9, sodium/potassium-transporting ATPase subunit gamma, immunoglobulin kappa variable 2-28, oncoprotein-induced transcript 3 protein, serglycin, coagulation factor XII, coagulation factor XIII A chain, insulin, histidine-rich glycoprotein, immunoglobulin kappa variable 3-11, immunoglobulin kappa variable 1-39, collagen alpha-1(I) chain, inter-alpha-trypsin inhibitor heavy chain H5, latent-transforming growth factor beta-binding protein 2, integrin alpha-IIb, membrane-associated progesterone receptor component 1, immunoglobulin lambda variable 6-57, immunoglobulin kappa variable 3-15, complement C1r subcomponent-like protein, histone H1.2, rho GDP-dissociation inhibitor 2, latent-transforming growth factor beta-binding protein 4, collagen alpha-1(XVIII) chain, immunoglobulin lambda variable 2-18, zinc-alpha-2-glycoprotein, talin-1, secretogranin-1, neutrophil defensin 3, cytochrome P450 2E1, gastric inhibitory polypeptide, immunoglobulin heavy variable 3-15, immunoglobulin lambda variable 2-11, transcription initiation factor TFIID subunit 1, collagen alpha-1(VII) chain, integral membrane protein 2B, pigment epithelium-derived factor, voltage-dependent N-type calcium channel subunit alpha-1B, immunoglobulin lambda variable 3-27, ras GTPase-activating protein nGAP, keratin, type I cytoskeletal 17, tubulin beta chain, sulfhydryl oxidase 1, immunoglobulin kappa variable 4-1, complement C1r subcomponent, homeobox protein Hox-B2, transcription factor SOX-10, E3 ubiquitin-protein ligase SIAH2, decorin, SPARC, type I collagen alpha-1 chain, type IV collagen alpha-1 chain, laminin gamma 1 chain, vimentin, type III collagen, type IV collagen alpha-3 chain, type VII collagen alpha-1 chain, type VI collagen alpha-1 chain, type V collagen alpha-1 chain, nidogen-1, and type VI collagen alpha-3 chain.

In some embodiments of the therapeutic agent, the cleavage sequence of the reporter polypeptide is a cleavage sequence set forth in Column II or III of Table A (or a subset thereof). In some embodiments, the cleavage sequence does not comprise a methionine residue immediately N-terminal to a scissile bond (contained therein), when the methionine is the first residue at N terminus of the reporter polypeptide. In some embodiments, the target tissue or cell is characterized by an increased amount or activity of the mammalian protease in proximity to the target tissue or cell as compared to a non-target tissue or cell in the subject. In some embodiments, the mammalian proatease is produced at the target tissue or cell. In some embodiments, the peptide substrate comprises an amino acid sequence having at most three amino acid substitutions, or at most two amino acid substitutions, or at most one amino acid substitution with respect to a sequence set forth in Column II or III of Table A (or a subset thereof). In some embodiments, the peptide substrate comprises an amino acid sequence having at most three amino acid substitutions with respect to a sequence set forth in Column II or III of Table A (or a subset thereof). In some embodiments, the scissile bond is not immediately C-terminal to a methionine residue.

In some embodiments of the therapeutic agent, the peptide substrate contains from six to twenty-five or six to twenty amino acid residues. In some embodiments of the therapeutic agent, the peptide substrate contains from six to twenty-five amino acid residues. In some embodiments of the therapeutic agent, the peptide substrate contains from six to twenty amino acid residues. In some embodiments, the peptide substrate contains from seven to twelve amino acid residues. In some embodiments, the peptide substrate comprises an amino acid sequence having at most two amino acid substitutions with respect to a sequence set forth in Column II or III of Table A (or a subset thereof). In some embodiments, the peptide substrate comprises an amino acid sequence having at most one amino acid substitution with respect to a sequence set forth in Column II or III of Table A (or a subset thereof). In some embodiments, none of the at most three amino acid substitutions, or the at most two amino acid substitutions, or the at most one amino acid substitution is at a position corresponding to an amino acid residue immediately adjacent to a corresponding scissile bond of the corresponding sequence shown in Column II or III of Table A (or a subset thereof). In some embodiments, the peptide substrate comprises an amino acid sequence identical to a sequence set forth in Column II or III of Table A (or a subset thereof). In some embodiments, the peptide substrate does not comprise a methionine residue immediately N-terminal to a scissile bond (contained therein). In some embodiments, the peptide substrate does not comprise an amino acid sequence selected from the group consisting of #279, #280, #282, #283, #298, #299, #302, #303, #305, #307, #308, #349, #396, #397, #416, #417, #418, #458, #459, #460, #466, #481 and #482 (or any combination thereof) of Column II of Table A. In some embodiments, the peptide substrate comprises two or three sequences set forth in Column II or III of Table A (or a subset thereof). In some embodiments, where the peptide substrate comprises two sequences set forth in Column II or III of Table A (or a subset thereof), the two sequences partially overlap one another. In some embodiments, where the peptide substrate comprises two sequences set forth in Column II or III of Table A (or a subset thereof), the two sequences do not overlap one another. In some embodiments, where the peptide substrate comprises three sequences set forth in Column II or III of Table A (or a subset thereof), two or all of the three sequences do not overlap one another. In some embodiments, where the peptide substrate comprises three sequences set forth in Column II or III of Table A (or a subset thereof), one of the three sequences partially overlaps with another sequence or both other sequences of the three sequences. In some embodiments, where the peptide substrate comprises three sequences set forth in Column II or III of Table A (or a subset thereof), two of the three sequences partially overlap with one another. In some embodiments, where the peptide substrate comprises three sequences set forth in Column II or III of Table A (or a subset thereof), each two of the three sequences partially overlap with one another. In some embodiments, where the peptide substrate comprises three sequences set forth in Column II or III of Table A (or a subset thereof), all of the three sequences partially overlap with one another. In some embodiments, the peptide substrate susceptible to cleavage by the mammalian protease is susceptible to cleavage by a plurality of mammalian proteases comprising the mammalian protease. In some embodiments, the peptide substrate susceptible to cleavage by the plurality of mammalian proteases has at most three amino acid substitutions, or at most two amino acid substitutions, or at most one amino acid substitution with respect to a sequence set forth in Table 1(j). In some embodiments, the peptide substrate susceptible to cleavage by the plurality of mammalian proteases has at most three amino acid substitutions with respect to a sequence set forth in Table 1(j). In some embodiments, the peptide substrate susceptible to cleavage by the plurality of mammalian proteases has at most two amino acid substitutions with respect to a sequence set forth in Table 1(j). In some embodiments, the peptide substrate susceptible to cleavage by the plurality of mammalian proteases has at most one amino acid substitution with respect to a sequence set forth in Table 1(j). In some embodiments, none of the at most three amino acid substitutions, or the at most two amino acid substitutions, or the at most one amino acid substitution is at a position corresponding to an amino acid residue immediately adjacent to a corresponding scissile bond of the corresponding sequence set forth in Table 1(j). In some embodiments, the peptide substrate susceptible to cleavage by the plurality of mammalian proteases comprises a sequence set forth in Table 1(j).

In some embodiments of the therapeutic agent, the release segment (RS) is capable of being cleaved when in proximity to a target tissue or cell, and wherein the target tissue or cell produces the mammalian protease for which the RS is a peptide substrate. In some embodiments, the mammalian protease for cleavage of the release segment (RS) is a serine protease, a cysteine protease, an aspartate protease, a threonine protease, or a metalloproteinase. In some embodiments, the mammalian protease for cleavage of the release segment (RS) is selected from the group consisting of disintegrin and metalloproteinase domain-containing protein 10 (ADAM10), disintegrin and metalloproteinase domain-containing protein 12 (ADAM12), disintegrin and metalloproteinase domain-containing protein 15 (ADAM15), disintegrin and metalloproteinase domain-containing protein 17 (ADAM17), disintegrin and metalloproteinase domain-containing protein 9 (ADAM9), disintegrin and metalloproteinase with thrombospondin motifs 5 (ADAMTS5), Cathepsin B, Cathepsin D, Cathepsin E, Cathepsin K, cathepsin L, cathepsin S, Fibroblast activation protein alpha, Hepsin, kallikrein-2, kallikrein-4, kallikrein-3, Prostate-specific antigen (PSA), kallikrein-13, Legumain, matrix metallopeptidase 1 (MMP-1), matrix metallopeptidase 10 (MMP-10), matrix metallopeptidase 11 (MMP-11), matrix metallopeptidase 12 (MMP-12), matrix metallopeptidase 13 (MMP-13), matrix metallopeptidase 14 (MMP-14), matrix metallopeptidase 16 (MMP-16), matrix metallopeptidase 2 (MMP-2), matrix metallopeptidase 3 (MMP-3), matrix metallopeptidase 7 (MMP-7), matrix metallopeptidase 8 (MMP-8), matrix metallopeptidase 9 (MMP-9), matrix metallopeptidase 4 (MMP-4), matrix metallopeptidase 5 (MMP-5), matrix metallopeptidase 6 (MMP-6), matrix metallopeptidase 15 (MMP-15), neutrophil elastase, protease activated receptor 2 (PAR2), plasmin, prostasin, PSMA-FOLH1, membrane type serine protease 1 (MT-SP1), matriptase, and u-plasminogen. In some embodiments, the mammalian protease for cleavage of the release segment (RS) is selected from the group consisting of matrix metallopeptidase 1 (MMP1), matrix metallopeptidase 2 (MMP2), matrix metallopeptidase 7 (MMP1), matrix metallopeptidase 9 (MMP9), matrix metallopeptidase 11 (MMP11), matrix metallopeptidase 14 (MMP14), urokinase-type plasminogen activator (uPA), legumain, and matriptase.

In some embodiments of the therapeutic agent, the therapeutic agent further comprises a masking moiety (MM) linked, directly or indirectly, to the release segment (RS). In some embodiments, the therapeutic agent, in an uncleaved state, has a structural arrangement from N-terminus to C-terminus of BM-RS-MM or MM-RS-BM. In some embodiments of the therapeutic agent, upon cleavage of the release segment (RS), the masking moiety (MM) is released from the therapeutic agent. In some embodiments, the masking moiety (MM) comprises an extended recombinant polypeptide (XTEN). In some embodiments, the XTEN is characterized in that: (i) it comprises at least 100 amino acids; (ii) at least 90% of the amino acid residues of it are selected from glycine (G), alanine (A), serine (S), threonine (T), glutamate (E) and proline (P); and (iii) it comprises at least 4 different types of amino acids selected from G, A, S, T, E, and P. In some embodiments, the extended recombinant polypeptide (XTEN) comprises an amino acid sequence having at least 90%, 91%, 92%, 93%, 94%, 95%, 96%, 97%, 98%, 99%, or 100% sequence identity to a sequence set forth in Tables 2b-2c. In some embodiments, the masking moiety (MM), when linked to the therapeutic agent, interferes with an interaction of the biologically active moiety (BM) to the target tissue or cell such that a dissociation constant (K_{d}) of the BM of the therapeutic agent with a target cell marker borne by the target tissue or cell is greater, when the therapeutic agent is in an uncleaved state, compared to a dissociation constant (Kd) of a corresponding biologically active moiety with the target cell marker. In some embodiments, the therapeutic agent effects a broader therapeutic window in delivery of the BM to the target tissue or cell compared to a corresponding biologically active moiety. In some embodiments, the therapeutic agent has a longer terminal half-life compared to that of a corresponding biologically active moiety. In some embodiments, the therapeutic agent is less immunogenic compared to a corresponding biologically active moiety. In some embodiments, the immunogenicity is ascertained by measuring production of IgG antibodies that selectively bind to the biologically active moiety after administration of comparable doses to a subject. In some embodiments, the therapeutic agent has a greater apparent molecular weight factor under a physiological condition compared to a corresponding biologically active moiety.

In some embodiments of the therapeutic agent, the release segment (RS) is a first release segment (RS1), wherein the scissile bond is a first scissile bond, and wherein the therapeutic agent further comprises a second release segment (RS2) linked, directly or indirectly, to the biologically active moiety (BM), wherein the RS2 comprises a second peptide substrate or cleavage by a mammalian protease at a second scissile bond. In some embodiments, the mammalian protease for cleavage of the RS2 is identical to the mammalian protease for cleavage of the RS1. In some embodiments, the mammalian protease for cleavage of the RS2 is different from the mammalian protease for cleavage of the RS1. In some embodiments, the RS2 has an amino acid sequence identical to that of the RS1. In some embodiments, the RS2 has an amino acid sequence different from that of the RS1. In some embodiments, each of the RS1 and the RS2 comprises a peptide substrate for a different mammalian protease selected from the group consisting of disintegrin and metalloproteinase domain-containing protein 10 (ADAM10), disintegrin and metalloproteinase domain-containing protein 12 (ADAM12), disintegrin and metalloproteinase domain-containing protein 15 (ADAM15), disintegrin and metalloproteinase domain-containing protein 17 (ADAM17), disintegrin and metalloproteinase domain-containing protein 9 (ADAM9), disintegrin and metalloproteinase with thrombospondin motifs 5 (ADAMTS5), Cathepsin B, Cathepsin D, Cathepsin E, Cathepsin K, cathepsin L, cathepsin S, Fibroblast activation protein alpha, Hepsin, kallikrein-2, kallikrein-4, kallikrein-3, Prostate-specific antigen (PSA), kallikrein-13, Legumain, matrix metallopeptidase 1 (MMP-1), matrix metallopeptidase 10 (MMP-10), matrix metallopeptidase 11 (MMP-11), matrix metallopeptidase 12 (MMP-12), matrix metallopeptidase 13 (MMP-13), matrix metallopeptidase 14 (MMP-14), matrix metallopeptidase 16 (MMP-16), matrix metallopeptidase 2 (MMP-2), matrix metallopeptidase 3 (MMP-3), matrix metallopeptidase 7 (MMP-7), matrix metallopeptidase 8 (MMP-8), matrix metallopeptidase 9 (MMP-9), matrix metallopeptidase 4 (MMP-4), matrix metallopeptidase 5 (MMP-5), matrix metallopeptidase 6 (MMP-6), matrix metallopeptidase 15 (MMP-15), neutrophil elastase, protease activated receptor 2 (PAR2), plasmin, prostasin, PSMA-FOLH1, membrane type serine protease 1 (MT-SP1), matriptase, and u-plasminogen. In some embodiments, each of the RS1 and the RS2 comprises a peptide substrate for a different mammalian protease selected from the group consisting of matrix metallopeptidase 1 (MMP1), matrix metallopeptidase 2 (MMP2), matrix metallopeptidase 7 (MMP1), matrix metallopeptidase 9 (MMP9), matrix metallopeptidase 11 (MMP11), matrix metallopeptidase 14 (MMP14), urokinase-type plasminogen activator (uPA), legumain, and matriptase. In some embodiments, the second scissile bond is not immediately C-terminal to a methionine residue.

In some embodiments of the therapeutic agent, the second peptide substrate contains from six to twenty-five or six to twenty amino acid residues. In some embodiments of the therapeutic agent, the second peptide substrate contains from six to twenty-five amino acid residues. In some embodiments of the therapeutic agent, the second peptide substrate contains from six to twenty amino acid residues. In some embodiments, the second peptide substrate contains from seven to twelve amino acid residues. In some embodiments, the second peptide substrate comprises an amino acid sequence having at most three amino acid substitutions, or at most two amino acid substitutions, or at most one amino acid substitution with respect to a sequence set forth in Column II or III of Table A (or a subset thereof). In some embodiments, the second peptide substrate comprises an amino acid sequence having at most three amino acid substitutions with respect to a sequence set forth in Column II or III of Table A (or a subset thereof). In some embodiments, the second peptide substrate comprises an amino acid sequence having at most two amino acid substitutions with respect to a sequence set forth in Column II or III of Table A (or a subset thereof). In some embodiments, the second peptide substrate comprises an amino acid sequence having at most one amino acid substitution with respect to a sequence set forth in Column II or III of Table A (or a subset thereof). In some embodiments, none of the at most three amino acid substitutions, or the at most two amino acid substitutions, or the at most one amino acid substitution (of the second peptide substrate) is at a position corresponding to an amino acid residue immediately adjacent to a corresponding scissile bond of the corresponding sequence shown in Column II or III of Table A (or a subset thereof). In some embodiments, the second peptide substrate comprises an amino acid sequence identical to a sequence set forth in Column II or III of Table A (or a subset thereof). In some embodiments, the second peptide substrate does not comprise a methionine residue immediately N-terminal to a scissile bond (contained therein). In some embodiments, the second peptide substrate does not comprise an amino acid sequence selected from the group consisting of #279, #280, #282, #283, #298, #299, #302, #303, #305, #307, #308, #349, #396, #397, #416, #417, #418, #458, #459, #460, #466, #481 and #482 (or any combination thereof) of Column II of Table A. In some embodiments, the second peptide substrate comprises two or three sequences set forth in Column II or III of Table A (or a subset thereof). In some embodiments, where the second peptide substrate comprises two sequences set forth in Column II or III of Table A (or a subset thereof), the two sequences (of the second peptide substrate) partially overlap one another. In some embodiments, where the second peptide substrate comprises two sequences set forth in Column II or III of Table A (or a subset thereof), the two sequences (of the second peptide substrate) do not overlap one another. In some embodiments, where the second peptide substrate comprises three sequences set forth in Column II or III of Table A (or a subset thereof), two or all of the three sequences (of the second peptide substrate) do not overlap one another. In some embodiments, where the second peptide substrate comprises three sequences set forth in Column II or III of Table A (or a subset thereof), one of the three sequences (of the second peptide substrate) partially overlaps with another sequence or both other sequences of the three sequences (of the second peptide substrate). In some embodiments, where the second peptide substrate comprises three sequences set forth in Column II or III of Table A (or a subset thereof), two of the three sequences (of the second peptide substrate) partially overlap with one another. In some embodiments, where the second peptide substrate comprises three sequences set forth in Column II or III of Table A (or a subset thereof), each two of the three sequences (of the second peptide substrate) partially overlap with one another. In some embodiments, where the second peptide substrate comprises three sequences set forth in Column II or III of Table A (or a subset thereof), all of the three sequences (of the second peptide substrate) partially overlap with one another. In some embodiments, the second peptide substrate susceptible to cleavage by the mammalian protease is susceptible to cleavage by a plurality of mammalian proteases comprising the mammalian protease. In some embodiments, the second peptide substrate susceptible to cleavage by the plurality of mammalian proteases has at most three amino acid substitutions, or at most two amino acid substitutions, or at most one amino acid substitution with respect to a sequence set forth in Table 1(j). In some embodiments, the second peptide substrate susceptible to cleavage by the plurality of mammalian proteases has at most three amino acid substitutions with respect to a sequence set forth in Table 1(j). In some embodiments, the second peptide substrate susceptible to cleavage by the plurality of mammalian proteases has at most two amino acid substitutions with respect to a sequence set forth in Table 1(j). In some embodiments, the second peptide substrate susceptible to cleavage by the plurality of mammalian proteases has at most one amino acid substitution with respect to a sequence set forth in Table 1(j). In some embodiments, none of the at most three amino acid substitutions, or the at most two amino acid substitutions, or the at most one amino acid substitution (of the second peptide substrate) is at a position corresponding to an amino acid residue immediately adjacent to a corresponding scissile bond of the corresponding sequence set forth in Table 1(j). In some embodiments, the second peptide substrate susceptible to cleavage by the plurality of mammalian proteases comprises a sequence set forth in Table 1(j).

In some embodiments of the therapeutic agent, the second release segment (RS2) is capable of being cleaved when in proximity to the target tissue or cell, and wherein the target tissue or cell produces the mammalian protease for which the RS2 is a peptide substrate. This includes tumor produced proteases and tumor melieu produced proteases. In some embodiments, the mammalian protease for cleavage of the second release segment (RS2) is a serine protease, a cysteine protease, an aspartate protease, a threonine protease or a metalloproteinase. In some embodiments, the mammalian protease for cleavage of the release segment (RS) is selected from the group consisting of disintegrin and metalloproteinase domain-containing protein 10 (ADAM10), disintegrin and metalloproteinase domain-containing protein 12 (ADAM12), disintegrin and metalloproteinase domain-containing protein 15 (ADAM15), disintegrin and metalloproteinase domain-containing protein 17 (ADAM17), disintegrin and metalloproteinase domain-containing protein 9 (ADAM9), disintegrin and metalloproteinase with thrombospondin motifs 5 (ADAMTS5), Cathepsin B, Cathepsin D, Cathepsin E, Cathepsin K, cathepsin L, cathepsin S, Fibroblast activation protein alpha, Hepsin, kallikrein-2, kallikrein-4, kallikrein-3, Prostate-specific antigen (PSA), kallikrein-13, Legumain, matrix metallopeptidase 1 (MMP-1), matrix metallopeptidase 10 (MMP-10), matrix metallopeptidase 11 (MMP-11), matrix metallopeptidase 12 (MMP-12), matrix metallopeptidase 13 (MMP-13), matrix metallopeptidase 14 (MMP-14), matrix metallopeptidase 16 (MMP-16), matrix metallopeptidase 2 (MMP-2), matrix metallopeptidase 3 (MMP-3), matrix metallopeptidase 7 (MMP-7), matrix metallopeptidase 8 (MMP-8), matrix metallopeptidase 9 (MMP-9), matrix metallopeptidase 4 (MMP-4), matrix metallopeptidase 5 (MMP-5), matrix metallopeptidase 6 (MMP-6), matrix metallopeptidase 15 (MMP-15), neutrophil elastase, protease activated receptor 2 (PAR2), plasmin, prostasin, PSMA-FOLH1, membrane type serine protease 1 (MT-SP1), matriptase, and u-plasminogen. In some embodiments, the mammalian protease for cleavage of the second release segment (RS2) is selected from the group consisting of matrix metallopeptidase 1 (MMP1), matrix metallopeptidase 2 (MMP2), matrix metallopeptidase 7 (MMP1), matrix metallopeptidase 9 (MMP9), matrix metallopeptidase 11 (MMP11), matrix metallopeptidase 14 (MMP14), urokinase-type plasminogen activator (uPA), legumain, and matriptase.

In some embodiments of the therapeutic agent, the masking moiety (MM) is a first masking moiety (MM1), and wherein the therapeutic agent further comprises a second masking moiety (MM2) linked, directly or indirectly, to the second release segment (RS2). In some embodiments, the therapeutic agent, in an uncleaved state, has a structural arrangement from N-terminus to C-terminus of MM1-RS1-BM-RS2-MM2, MM1-RS2-BM-RS1-MM2, MM2-RS1-BM-RS2-MM1, or MM2-RS2-BM-RS1-MM1. In some embodiments of the therapeutic agent, upon cleavage of the second release segment (RS2), the second masking moiety (MM2) is released from the therapeutic agent. In some embodiments, the second masking moiety (MM2) comprises a second extended recombinant polypeptide (XTEN2). In some embodiments, the XTEN2 is characterized in that: (i) it comprises at least 100 amino acids; (ii) at least 90% of the amino acid residues of it are selected from glycine (G), alanine (A), serine (S), threonine (T), glutamate (E) and proline (P); and (iii) it comprises at least 4 different types of amino acids selected from G, A, S, T, E, and P. In some embodiments, the XTEN2 comprises an amino acid sequence having at least 90%, 91%, 92%, 93%, 94%, 95%, 96%, 97%, 98%, 99%, or 100% sequence identity to a sequence selected from the group of sequences set forth in Tables 2b-2c. In some embodiments, the first masking moiety (MM1) and the second masking moiety (MM2), when both linked in the therapeutic agent, interfere with an interaction of the biologically active moiety (BM) to the target tissue or cell such that a dissociation constant (K_{d}) of the BM of the therapeutic agent with a target cell marker borne by the target tissue or cell is greater, when the therapeutic agent is in an uncleaved state, compared to a dissociation constant (Kd) of a corresponding biologically active moiety. In some embodiments, the therapeutic agent, in which the biologically active moiety (BM) is linked, directly or indirectly, to one or both of the first masking moiety (MM1) and the second masking moiety (MM2), effects a broader therapeutic window in delivery of the BM to the target tissue or cell compared to a corresponding biologically active moiety. In some embodiments, the therapeutic agent, in which the biologically active moiety (BM) is linked, directly or indirectly, to one or both of the first masking moiety (MM1) and the second masking moiety (MM2), has a longer terminal half-life compared to that of a corresponding biologically active moiety. In some embodiments, the therapeutic agent, in which the biologically active moiety (BM) is linked, directly or indirectly, to one or both of the first masking moiety (MM1) and the second masking moiety (MM2), is less immunogenic compared to a corresponding biologically active moiety. In some embodiments of the therapeutic agent, immunogenicity is ascertained by measuring production of IgG antibodies that selectively bind to the biologically active moiety after administration of comparable doses to a subject. In some embodiments, the therapeutic agent, in which the biologically active moiety (BM) is linked, directly or indirectly, to one or both of the first masking moiety (MM1) and the second masking moiety (MM2), has a greater apparent molecular weight factor under a physiological condition compared to a corresponding biologically active moiety. In some embodiments, the therapeutic agent comprises a fusion polypeptide or conjugate.

In some embodiments of the therapeutic agent, the biologically active moiety (BM) comprises a biologically active peptide (BP). In some embodiments, the BP comprises an antibody, a cytokine, a cell receptor, or a fragment thereof.

In some embodiments, the therapeutic agent comprises a recombinant polypeptide. In some embodiments, the recombinant polypeptide comprises the biologically active peptide (BP) and the release segment (RS). In some embodiments, the recombinant polypeptide comprises the biologically active peptide (BP), the release segment (RS), and the masking moiety (MM). In some embodiments, the recombinant polypeptide, in an uncleaved state, has a structural arrangement from N-terminus to C-terminus of BP-RS-MM or MM-RS-BP. In some embodiments, the recombinant polypeptide comprises the biologically active peptide (BP), the first release segment (RS1), and the second release segment (RS2). In some embodiments, the recombinant polypeptide comprises the biologically active peptide (BP), the first release segment (RS1), the second release segment (RS2), the first masking moiety (MM1), and the second masking moiety (MM2). In some embodiments, the recombinant polypeptide, in an uncleaved state, has a structural arrangement from N-terminus to C-terminus of MM1-RS1-BP-RS2-MM2, MM1-RS2-BP-RS1-MM2, MM2-RS1-BP-RS2-MM1, or MM2-RS2-BP-RS1-MM1. In some embodiments, the recombinant polypeptide comprises the biologically active peptide (BP), the first release segment (RS1), the second release segment (RS2), the first extended recombinant polypeptide (XTEN1), and the second extended recombinant polypeptide (XTEN2). In some embodiments, the recombinant polypeptide, in an uncleaved state, has a structural arrangement from N-terminus to C-terminus of XTEN1-RS1-BP-RS2-XTEN2, XTEN1-RS2-BP-RS1-XTEN2, XTEN2-RS1-BP-RS2-XTEN1, or XTEN2-RS2-BP-RS1-XTEN1.

In some embodiments of the therapeutic agent, the biologically active polypeptide (BP) comprises a binding moiety having a binding affinity for a target cell marker on the target tissue or cell. In some embodiments, the target cell marker is an effector cell antigen expressed on a surface of an effector cell. In some embodiments, the binding moiety is an antibody. In some embodiments, the binding moiety is an antibody selected from the group consisting of Fv, Fab, Fab′, Fab′-SH, nanobody (also known as single domain antibody or V_{HH}), linear antibody, and single-chain variable fragment (scFv). In some embodiments, the binding moiety is a first binding moiety, wherein the target cell marker is a first target cell marker, and wherein the biologically active polypeptide (BP) further comprises a second binding moiety linked, directly or indirectly to the first binding moiety, wherein the second binding moiety has a binding affinity for a second target cell marker on the target tissue or cell. In some embodiments, the second target cell marker is a marker on a tumor cell or a cancer cell. In some embodiments, the second binding moiety is an antibody. In some embodiments, the second binding moiety is an antibody selected from the group consisting of Fv, Fab, Fab′, Fab′-SH, nanobody (also known as single domain antibody or V_{HH}), linear antibody, and single-chain variable fragment (scFv).

Certain aspects of the present disclosure provide an isolated nucleic acid, the isolated nucleic acid comprising: (a) a polynucleotide encoding a recombinant polypeptide as described herein; or (b) a reverse complement of the polynucleotide of (a).

Certain aspects of the present disclosure provide an expression vector, the expression vector comprising a polynucleotide sequence as described herein and a recombinant regulatory sequence operably linked to the polynucleotide sequence.

Certain aspects of the present disclosure provide an isolated host cell, the isolated cell comprising the expression vector as described herein. In some embodiments, the host cell is a prokaryote. In some embodiments, the host cell is E. coli or a mammalian cell. In some embodiments, the host cell is E. coli. In some embodiments, the host cell is a mammalian cell.

Some aspects of the present disclosure provide a pharmaceutical composition, the pharmaceutical composition comprising a therapeutic agent as described herein and one or more pharmaceutically suitable excipients. In some embodiments, the pharmaceutical composition is formulated for oral, intradermal, subcutaneous, intravenous, intra-arterial, intraabdominal, intraperitoneal, intrathecal, or intramuscular administration. In some embodiments, the pharmaceutical composition is in a liquid form or frozen form. In some embodiments, the pharmaceutical composition is in a pre-filled syringe for a single injection. In some embodiments, the pharmaceutical composition is formulated as a lyophilized powder to be reconstituted prior to administration.

Some aspects of the present disclosure provide a kit, the kit comprising a pharmaceutical composition as described herein, a container, and a label or package insert on or associated with the container.

In certain aspects, the present disclosure provides a method for preparing a therapeutic agent (e.g., activatable therapeutic agent, or non-natural, activatable therapeutic agent) as provided herein.

In certain aspects, the present disclosure provides a method for preparing a therapeutic agent (e.g., activatable therapeutic agent, or non-natural, activatable therapeutic agent), the method comprising:
(a) culturing a host cell comprising a nucleic acid construct that encodes a recombinant polypeptide under conditions sufficient to express the recombinant polypeptide in the host cell, wherein the recombinant polypeptide comprises a biologically active polypeptide (BP), a release segment (RS), and a masking moiety (MM), wherein:
   the RS comprises a peptide substrate susceptible for cleavage by a mammalian protease at a scissile bond, wherein the peptide substrate comprises an amino acid sequence having at most three or two amino acid substitutions (or at most one amino acid substitution) with respect to a sequence set forth in Column II or III of Table A (or a subset thereof); and
   the recombinant polypeptide has a structural arrangement from N-terminus to C-terminus of BP-RS-MM or MM-RS-BP; and
(b) recovering the therapeutic agent (e.g., activatable therapeutic agent, or non-natural, activatable therapeutic agent) comprising the recombinant polypeptide.

In some embodiments of the method for preparing the therapeutic agent, the peptide substrate susceptible to cleavage by the mammalian protease is susceptible to cleavage by a plurality of mammalian proteases comprising the mammalian protease. In some embodiments, the peptide substrate susceptible to cleavage by the plurality of mammalian proteases has at most three amino acid substitutions, or at most two amino acid substitutions, or at most one amino acid substitution with respect to a sequence set forth in Table 1(j). In some embodiments, the peptide substrate susceptible to cleavage by the plurality of mammalian proteases comprises a sequence set forth in Table 1(j). In some embodiments, the peptide substrate does not comprise SEQ ID NO: 1. In some embodiments, the peptide substrate does not comprise SEQ ID NO: 2. In some embodiments, the peptide substrate does not comprise SEQ ID NO: 3. In some embodiments, the peptide substrate does not comprise SEQ ID NO: 4. In some embodiments, the peptide substrate does not comprise SEQ ID NO: 5. In some embodiments, the peptide substrate does not comprise SEQ ID NO: 6. In some embodiments, the peptide substrate does not comprise SEQ ID NO: 7. In some embodiments, the peptide substrate does not comprise SEQ ID NO: 8. In some embodiments, the masking moiety (MM) comprises an extended recombinant polypeptide (XTEN).

In some embodiments of the method for preparing the therapeutic agent, the release segment (RS) is a first release segment (RS1), wherein the peptide substrate is a first peptide substrate, wherein the scissile bond is a first scissile bond, wherein the masking moiety (MM) is a first masking moiety (MM1), and wherein the recombinant polypeptide further comprises a second release segment (RS2), and a second masking moiety (MM2), wherein: the RS2 comprises a second peptide substrate susceptible for cleavage by a mammalian protease at a second scissile bond, wherein the second peptide substrate comprises an amino acid sequence having at most three amino acid substitutions, or at most two amino acid substitutions, or at most one amino acid substitution with respect to a sequence set forth in Column II or III of Table A (or a subset thereof); and the recombinant polypeptide has a structural arrangement from N-terminus to C-terminus of MM1-RS1-BP-RS2-MM2, MM1-RS2-BP-RS1-MM2, MM2-RS1-BP-RS2-MM1, or MM2-RS2-BP-RS1-MM1.

In some embodiments of the method for preparing the therapeutic agent, the second peptide substrate susceptible to cleavage by the mammalian protease is susceptible to cleavage by a plurality of mammalian proteases comprising the mammalian protease. In some embodiments, the second peptide substrate susceptible to cleavage by the plurality of mammalian proteases has at most three amino acid substitutions, or at most two amino acid substitutions, or at most one amino acid substitution with respect to a sequence set forth in Table 1(j). In some embodiments, the second peptide substrate susceptible to cleavage by the plurality of mammalian proteases comprises a sequence set forth in Table 1(j). In some embodiments, the second peptide substrate does not comprise SEQ ID NO: 1. In some embodiments, the second peptide substrate does not comprise SEQ ID NO: 2. In some embodiments, the second peptide substrate does not comprise SEQ ID NO: 3. In some embodiments, the second peptide substrate does not comprise SEQ ID NO: 4. In some embodiments, the second peptide substrate does not comprise SEQ ID NO: 5. In some embodiments, the second peptide substrate does not comprise SEQ ID NO: 6. In some embodiments, the second peptide substrate does not comprise SEQ ID NO: 7. In some embodiments, the second peptide substrate does not comprise SEQ ID NO: 8. In some embodiments, one of the first masking moiety (MM1) and the second masking moiety (MM2) comprises an extended recombinant polypeptide (XTEN). In some embodiments, the extended recombinant polypeptide (XTEN) is characterized in that: (i) it comprises at least 100 amino acids; (ii) at least 90% of the amino acid residues of it are selected from glycine (G), alanine (A), serine (S), threonine (T), glutamate (E) and proline (P); and (iii) it comprises at least 4 different types of amino acids selected from G, A, S, T, E, and P. In some embodiments, the extended recombinant polypeptide (XTEN) comprises an amino acid sequence having at least 90%, 91%, 92%, 93%, 94%, 95%, 96%, 97%, 98%, 99%, or 100% sequence identity to a sequence selected from the group set forth in Tables 2b-2c. In some embodiments, the extended recombinant polypeptide (XTEN) is a first extended recombinant polypeptide (XTEN1), and wherein the other one of the first masking moiety (MM1) and the second masking moiety (MM2) comprises a second extended recombinant polypeptide (XTEN2). In some embodiments, the second extended recombinant polypeptide (XTEN2) is characterized in that: (i) it comprises at least 100 amino acids; (ii) at least 90% of the amino acid residues of it are selected from glycine (G), alanine (A), serine (S), threonine (T), glutamate (E) and proline (P); and (iii) it comprises at least 4 different types of amino acids selected from G, A, S, T, E, and P. In some embodiments, the XTEN1 and the XTEN2 each comprise an amino acid sequence having at least 90%, 91%, 92%, 93%, 94%, 95%, 96%, 97%, 98%, 99%, or 100% sequence identity to a sequence selected from the group of sequences set forth in Tables 2b-2c.

In some embodiments of the method for preparing the therapeutic agent, the masking moiety (MM), when linked to the recombinant polypeptide, interferes with an interaction of the BP to a target tissue or cell such that a dissociation constant (K_{d}) of the BP of the recombinant polypeptide with a target cell marker borne by the target tissue or cell is greater, when the recombinant polypeptide is in an uncleaved state, compared to a dissociation constant (K_{d}) of a corresponding biologically active peptide, as measured in an in vitro assay under equivalent molar concentrations. In some embodiments, the first masking moiety (MM1) and the second masking moiety (MM2), when both linked in the recombinant polypeptide, interfere with an interaction of the BP to a target tissue or cell such that a dissociation constant (K_{d}) of the BP of the recombinant polypeptide with a target cell marker borne by the target tissue or cell is greater, when the recombinant polypeptide is in an uncleaved state, compared to a dissociation constant (K_{d}) of a corresponding biologically active peptide, as measured in an in vitro assay under equivalent molar concentrations. In some embodiments, the in vitro assay is selected from cell membrane integrity assay, mixed cell culture assay, cell-based competitive binding assay, FACS based propidium Iodide assay, trypan Blue influx assay, photometric enzyme release assay, radiometric 51Cr release assay, fluorometric Europium release assay, CalceinAM release assay, photometric MTT assay, XTT assay, WST-1 assay, alamar blue assay, radiometric 3H-Thd incorporation assay, clonogenic assay measuring cell division activity, fluorometric rhodamine123 assay measuring mitochondrial transmembrane gradient, apoptosis assay monitored by FACS-based phosphatidylserine exposure, ELISA-based TUNEL test assay, sandwich ELISA, caspase activity assay, cell-based LDH release assay, and cell morphology assay, or any combination thereof. In some embodiments, the activatable therapeutic agent is an activatable therapeutic agent or non-natural, activatable therapeutic agent as described herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates the nomenclature of a peptide biomarker sequence in a reporter polypeptide (e.g., a protein within or adjacent to a target tissue or cell from which a biomarker sequence is generated) (such as any set forth in Table A). The illustrative reporter polypeptide sequence comprises two cleavage sequences, a first cleavage sequence and a second cleavage sequence (such as any set forth in Table A), both capable of being recognized and cleaved by mammalian enzyme(s) (such as mammalian protease(s)). For example, in some cases, the first and second cleavage sequences can be recognized and cleaved by the same enzyme or the same set of enzymes. As another example, in some cases, the first and second cleavage sequences can be recognized and cleaved by different enzymes or different sets of enzymes. The first cleavage sequence contains a first scissile bond; and the second cleavage sequence, which is C-terminal to the first cleavage sequence, contains a second scissile bond. The first and second scissile bonds (such as indicated with hyphen (-) in Table A) divide the illustrative reporter polypeptide into three portions. By cleaving the illustrative reporter polypeptide with the corresponding enzyme(s) for which both the first and second cleavage sequences are substrates for, an N-terminal fragment (N-terminal to the first scissile bond), a center fragment (between the first and second scissile bonds), and a C-terminal fragment (C-terminal to the second scissile bond) can be obtained. The N-terminal, center, or C-terminal fragment (if present) (such as any set forth in Table A), or a derivative thereof, can function as a peptide biomarker sequence. The first or second cleavage sequence (such as any set forth in Table A) can be incorporated into a release segment of an activatable therapeutic agent (such as any described herein).

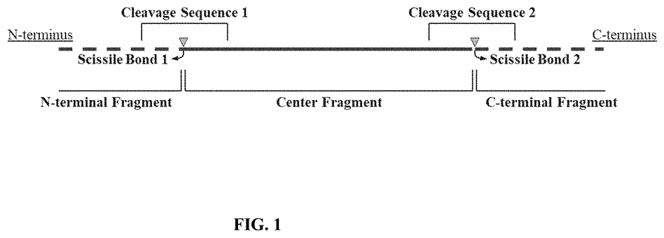

FIG. 2 illustrates the nomenclature of a peptide substrate and a scissile bond thereof for cleavage. The illustrative peptide substrate contains eight consecutive amino acid residues, of which four amino acid residues (with side chain groups, in the order from the N-terminus to the C-terminus, R₄, R₃, R₂, and R₁) are immediately N-terminal to the scissile bond and four amino acid residues (with side chain groups, in the order from the N-terminus to the C-terminus, R′₁, R′₂, R′₃, and R′₄) are immediately C-terminal to the scissile bond. For example, mammalian proteases can recognize up to four residues on both sides of the scissile bond. Upon cleavage, the illustrative peptide substrate separates into an N-terminal proteolytic fragment and a C-terminal proteolytic fragment. The four amino acid residues immediately N-terminal to the scissile bond in the illustrative peptide substrate forms the C-terminus of the N-terminal proteolytic fragment; and the four amino acid residues immediately C-terminal to the scissile bond in the illustrative peptide substrate forms the N-terminus of the C-terminal proteolytic fragment.

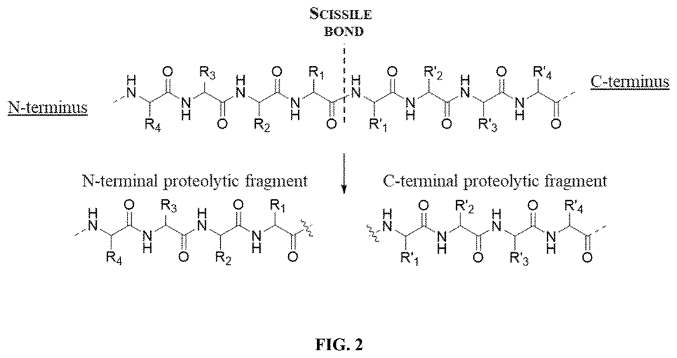

FIG. 3 illustrates a structural configuration of an exemplary activatable antibody (AA) composition comprising an antibody or a fragment thereof, a masking moiety (MM), and a release segment (RS).

FIG. 4 illustrates a structural configuration of an exemplary activatable antibody complex (AAC) composition with cross-masking occurring such that target binding by both antibodies or fragments thereof is attenuated in its uncleaved state, and target binding is increased upon cleavage of the release segment (RS) allowing the complex to disassemble. In this figure, the two antibodies or fragments thereof are referred to as the antibody domain 1 (ABD1) and antibody domain 2 (ABD2), respectively.

FIG. 5 illustrates a structural configuration of an exemplary activatable antibody complex (AAC) composition comprising two antibodies or fragments thereof, a masking moiety (MM), and a release segment (RS).

FIG. 6 illustrates a structural configuration of an exemplary activatable antibody complex (AAC) composition comprising four antibodies or fragments thereof, two masking moieties (MM) and three release segments (RS).

FIG. 7 illustrates a structural configuration of an exemplary activatable antibody composition (AA) comprising one antibody or antibody fragment (AB), two masking moieties (MM), and two release segments (RS).

FIG. 8 illustrates a structural configuration of an XTENylated Protease-Activated T-Cell Engager (XPAT). The illustrative XPAT comprises two binding moieties, each linked to an XTEN via a release segment.

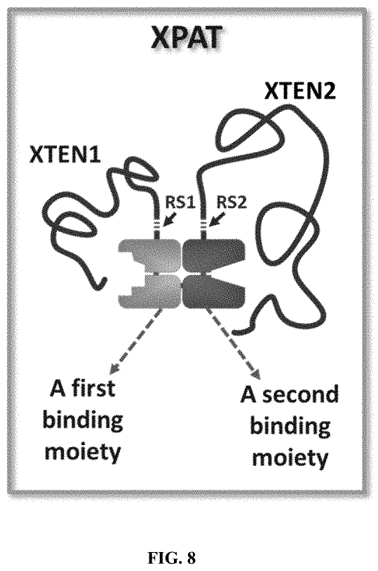

FIG. 9 illustrates the results of mammalian protease cleavage of release segments having sequence similarities to a sequence found in collagen I. The cleavage site is identified by a star (★) with portions of the sequences identical to the collagen site underlined. A sequence engineered not to be recognized or cleaved by proteases that recognize the collagen-derived cleavage site is set forth as 818-NonClv (RSR-3058) and amino acids that vary from the collagen sequence are shown in black type.

### DETAILED DESCRIPTION

In various cancer therapy modalities, agents have been generated that are conditionally activatable in the tumor microenvironment. However, there remains a need for developing more accurate and robust methods for predicting whether administration of these therapies will actually lead to therapeutic responses and outcomes upon administration of prodrugs or other activatable compositions. It is recognized that there is a cascade of events that leads to metastatic growth of cancer cells. A central factor in these events is the interaction between cancer cells and their microenvironment through which the tumor cells proliferate, build new vessels, leave the primary tumor bed and finally enter and persist at secondary sites of metastatic tumor growth. The extracellular matrix (ECM) of the tumor microenvironment consists of a variety of macromolecules, including collagen and glycoproteins. While the basement membranes of the ECM are formed mostly by type IV collagen, type I and type III collagen are the most abundant proteins of the underlying interstitial matrix. In healthy tissue, the ECM undergoes constant remodeling, mediated mainly by matrix-metalloproteinases (MMP), and matrix degradation is balanced by protein formation. This controlled remodeling of the ECM becomes disrupted in cancer development and progression.

In the process of MMP-mediated ECM degradation, small fragments of ECM turnover products are generated and released into the bloodstream. Several studies have shown that serum levels of collagen degradation fragments are elevated in cancer patients compared to healthy controls. Bager et al. found levels of MMP-degraded collagen type I, III and IV (i.e., C1M, C3M and C4M, respectively, Cancer Biomark. 2015; 15:783-788) to be 1.5 to 6-fold higher in ovarian and breast cancer patients than in controls. In the present invention, it is demonstrated that cleavage of the ECM by MMPs results in a cleavage product that is highly similar to the MMP cleavage site in protease-cleavable linkers in XPATs. The data presented herein demonstrate that the protease cleavable linker employed in the XPATs of this invention are more efficiently cleaved than the ECM by purified MMPs. As such, it is shown that the presence of ECM peptides in cancer patients can serve as an indicator that the patients' tumors have a microenvironment that has the appropriate protease (e.g., MMP) activity that can cleave the protease-cleavable linker in an XPAT. In this manner, the presence of the ECM peptides in the sample of a cancer patient thereby predicts whether a given patient or tumor will be able to cleave the XPAT and hence result in treatment of the tumor. This allows for a personalized approach to determine whether an XPAT will be cleaved in a given tumor type by determining whether the subject that has said tumor type has elevated plasma levels of certain cleavage product(s) derived from the extracellular matrix.

Before the embodiments of the disclosure are described, it is to be understood that such embodiments are provided by way of example only, and that various alternatives to the embodiments of the disclosure described herein may be employed in practicing the invention. Numerous variations, changes, and substitutions will now occur to those skilled in the art without departing from the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

### Definitions

In the context of the present application, the following terms have the meanings ascribed to them unless specified otherwise:

As used throughout the specification and claims, the terms “a”, “an” and “the” are generally used in the sense that they mean “at least one”, “at least a first”, “one or more” or “a plurality” of the referenced components or steps, except in instances wherein an upper limit is thereafter specifically stated. For example, a “cleavage sequence”, as used herein, means “at least a first cleavage sequence” but includes a plurality of cleavage sequences. The operable limits and parameters of combinations, as with the amounts of any single agent, will be known to those of ordinary skill in the art in light of the present disclosure.

The term “activatable,” as used herein with respect to a therapeutic agent, generally means that an activity or bioactivity of the therapeutic agent is capable of being enhanced upon activation, for example, via a physical, chemical or physiological process (e.g., enzymatic processes and metabolic processes).

As used herein, the term “activatable therapeutic agent,” generally refers to a therapeutic agent, of which an activity or bioactivity is capable of being enhanced upon activation, for example, via a physical, chemical or physiological process (e.g., enzymatic processes and metabolic processes). For example, the term “activatable therapeutic agent” may refer to a therapeutic agent in an inactive (or less active) state (at least inactive in one aspect) configured to be activated (i.e., in vitro, in vivo, or ex vivo) into an active (or more active) state (at least in the aspect that is inactive prior to activation). As another example, the term “activatable therapeutic agent” may refer to an active therapeutic agent (at least active in one aspect), of which an activity or bioactivity can be further enhanced (i.e., in vitro, in vivo, or ex vivo). Non-limiting examples of an activatable therapeutic agent include a prodrug, a probody, and a pro-moiety.

The terms “polypeptide”, “peptide”, and “protein” are used interchangeably herein to generally refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

As used herein in the context of the structure of a polypeptide, “N-terminus” (or “amino terminus”) and “C-terminus” (or “carboxyl terminus”) generally refer to the extreme amino and carboxyl ends of the polypeptide, respectively.

The term “N-terminal end sequence,” as used herein with respect to a polypeptide or polynucleotide sequence of interest, generally means that no other amino acid or nucleotide residues precede the N-terminal end sequence in the polypeptide or polynucleotide sequence of interest at the N-terminal end. The term “C-terminal end sequence,” as used herein with respect to a polypeptide or polynucleotide sequence of interest, generally means that no other amino acid or nucleotide residues follows the C-terminal end sequence in the polypeptide or polynucleotide sequence of interest at the C-terminal end.

The terms “non-naturally occurring” and “non-natural” are used interchangeably herein. The term “non-naturally occurring” or “non-natural,” as used herein with respect to a therapeutic agent, generally means that the agent is not biologically derived in mammals (including but not limited to human). The term “non-naturally occurring” or “non-natural,” as applied to sequences and as used herein, means polypeptide or polynucleotide sequences that do not have a counterpart to, are not complementary to, or do not have a high degree of homology with a wild-type or naturally-occurring sequence found in a mammal. For example, a non-naturally occurring polypeptide or fragment may share no more than 99%, 98%, 95%, 90%, 80%, 70%, 60%, 50% or even less amino acid sequence identity as compared to a natural sequence when suitably aligned.

As used herein, the term “antibody” generally refers to an immunoglobulin molecule, or any fragment thereof, which is immunologically reactive with an antigen of interest. For example, an antibody fragment may retain the ability to bind its ligand yet have a smaller molecular size and be in a single-chain format. The term “antibody” is used herein in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. The full-length antibodies may be for example monoclonal, recombinant, chimeric, deimmunized, humanized and human antibodies.

A “variant,” when applied to a biologically active protein is a protein with sequence homology to the native biologically active protein that retains at least a portion of the therapeutic and/or biological activity of the biologically active protein. For example, a variant protein may share at least 70%, 75%, 80%, 85%, 90%, 95%, 96%, 97%, 98% or 99% amino acid sequence identity compared with the reference biologically active protein. As used herein, the term “biologically active protein variant” includes proteins modified deliberately, as for example, by site directed mutagenesis, synthesis of the encoding gene, insertions, or accidentally through mutations and that retain activity.

The term “sequence variant” means polypeptides that have been modified compared to their native or original sequence by one or more amino acid insertions, deletions, or substitutions. Insertions may be located at either or both termini of the protein, and/or may be positioned within internal regions of the amino acid sequence. A non-limiting example is substitution of an amino acid in an XTEN with a different amino acid. In deletion variants, one or more amino acid residues in a polypeptide as described herein are removed. Deletion variants, therefore, include all fragments of a described polypeptide sequence. In substitution variants, one or more amino acid residues of a polypeptide are removed and replaced with alternative residues. In one aspect, the substitutions are conservative in nature and conservative substitutions of this type are well known in the art. In the context of an antibody or a biologically active polypeptide, a sequence variant would retain at least a portion of the binding affinity or biological activity, respectively, of the unmodified polypeptide.

The term “moiety” means a component of a larger composition or that is intended to be incorporated into a larger composition, such as a proteinaceous portion joined to a larger polypeptide as a contiguous or non-contiguous sequence. A moiety of a larger composition can confer a desired functionality. For example, an antibody fragment may retain the ability to bind its ligand yet have a smaller molecular size and be in a single-chain format. A masking moiety (including but not limited to an extended recombinant polypeptide (XTEN)) may confer the functionality of increasing molecular weight and/or half-life of a resulting larger composition with which the masking moiety is associated.

The terms “binding domain” and “binding moiety” are used interchangeably herein and each refer to a moiety having specific binding affinity to an antigen (such as an effector cell antigen, or a tumor-specific marker or an antigen of a target cell).

As used herein, a “release segment” or “RS” generally refers to a peptide with one or more cleavage sites in the sequence that can be recognized and cleaved by one or more mammalian enzymes (such as one or more proteases).

As used herein, a “peptide substrate” generally refers to an amino acid sequence recognized by an enzyme (such as a mammalian protease), leading to cleavage at a peptide bond (or the peptide bond) within the peptide substrate such that two consecutive amino acid residues connected by the peptide bond (or the scissile bond) prior to cleavage are separated upon cleavage. As used herein, a “scissile bond” generally refers to a peptide bond joining consecutive amino acids via an amide linkage that can be cleaved (or is cleaved) by an enzyme (such as a mammalian protease). For example, in the context of a peptide substrate, the scissile bond divides the peptide substrate into a C-terminal proteolytic fragment (or a C-terminal fragment) and an N-terminal proteolytic fragment (or an N-terminal fragment), where the C-terminal proteolytic fragment (or the C-terminal fragment) is N-terminal to the scissile bond in the peptide substrate and the N-terminal proteolytic fragment (or the N-terminal fragment) is C-terminal to the scissile bond in the peptide substrate. For example, the (putative) scissile bond of each cleavage sequence listed in Table A is indicated by a hyphen (-).

As used herein, the term “scissile bond” generally refers to a peptide bond between two amino acids which is capable of being cleaved by one or more proteases.

As used herein, the term “mammalian protease” generally means a protease that normally exists in the body fluids, cells, tissues, and may be found in higher levels in certain target tissues or cells, e.g., in diseased tissues (e.g., tumor) of a mammal.

The term “within”, when referring to a first polypeptide being linked to a second polypeptide, encompasses linking or fusion of an additional component that connects the N-terminus of the first or second polypeptide to the C-terminus of the second or first polypeptide, respectively, as well as insertion of the first polypeptide into the sequence of the second polypeptide. For example, when an RS component is linked “within” an recombinant polypeptide, the RS may be linked to the N-terminus, the C-terminus, or may be inserted between any two amino acids of an XTEN polypeptide.

The term “linked directly,” as used herein in the context of a therapeutic agent, generally refers to a structure in which a moiety is connected with or attached to another moiety without an intervening tether. The term “linked indirectly,” as used herein in the context of a therapeutic agent, generally refers to a structure in which a moiety of the therapeutic agent is connected with, or attached to, another moiety of the therapeutic agent via an intervening tether. The terms “link,” “linked,” and “linking,” as used herein in the context of a therapeutic agent, generally includes both covalent and non-covalent attachment of a moiety of the therapeutic agent to another moiety of the therapeutic agent.

“Activity” (such as “bioactivity”) as applied to form(s) of a composition provided herein, generally refers to an action or effect, including but not limited to receptor binding, antagonist activity, agonist activity, a cellular or physiologic response, cell lysis, cell death, or an effect generally known in the art for the effector component of the composition, whether measured by an in vitro, ex vivo or in vivo assay or a clinical effect.

“Effector cell”, as used herein, includes any eukaryotic cells capable of conferring an effect on a target cell. For example, an effect cell can induce loss of membrane integrity, pyknosis, karyorrhexis, apoptosis, lysis, and/or death of a target cell. In another example, an effector cell can induce division, growth, differentiation of a target cell or otherwise altering signal transduction of a target cell.

An “effector cell antigen” refers to molecules expressed by an effector cell, including without limitation cell surface molecules such as proteins, glycoproteins or lipoproteins. An effector cell antigen can serve as the binding counterpart of a binding moiety of the subject recombinant polypeptide.

As used herein, the term “ELISA” refers to an enzyme-linked immunosorbent assay as described herein or as otherwise known in the art.

A “host cell” generally includes an individual cell or cell culture which can be or has been a recipient for the subject vectors into which exogenous nucleic acid has been introduced, such as those described herein. Host cells include progeny of a single host cell. The progeny may not necessarily be completely identical (in morphology or in genomic of total DNA complement) to the original parent cell due to natural, accidental, or deliberate mutation. A host cell includes cells transfected in vivo with a vector of this disclosure.

The term “isolated”, when used to describe the various polypeptides disclosed herein, generally means polypeptide that has been identified and separated and/or recovered from a component of its natural environment or from a more complex mixture (such as during protein purification). Contaminant components of its natural environment are materials that would typically interfere with diagnostic or therapeutic uses for the polypeptide, and may include enzymes, hormones, and other proteinaceous or non-proteinaceous solutes. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is generally greater than that of its naturally occurring counterpart. In general, a polypeptide made by recombinant means and expressed in a host cell is considered to be “isolated.”

An “isolated nucleic acid” is a nucleic acid molecule that is identified and separated from at least one contaminant nucleic acid molecule with which it is ordinarily associated in the natural source of the polypeptide-encoding nucleic acid. For example, an isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal or extra-chromosomal location different from that of natural cells.

A “chimeric” protein or polypeptide contains at least one fusion polypeptide comprising at least one region in a different position in the sequence than that which occurs in nature. The regions may normally exist in separate proteins and are brought together in the fusion polypeptide; or they may normally exist in the same protein but are placed in a new arrangement in the fusion polypeptide. A chimeric protein may be created, for example, by chemical synthesis, or by recombinantly creating and translating a polynucleotide in which the peptide regions are encoded in the desired relationship.

The terms “fused” and “fusion” are used interchangeably herein, and refers to the joining together of two or more peptide or polypeptide sequences by recombinant means. A “fusion protein” or “chimeric protein” comprises a first amino acid sequence linked to a second amino acid sequence with which it is not naturally linked in nature.

“Uncleaved” and “uncleaved state” are used interchangeably herein, and refers to a polypeptide that has not been cleaved or digested by a protease such that the polypeptide remains intact.

“XTENylated” is used to denote a peptide or polypeptide that has been modified by the linking or fusion of one or more XTEN polypeptides (described, below) to the peptide or polypeptide, whether by recombinant or chemical cross-linking means.

“Crosslinking,” and “conjugating,” are used interchangeably herein, and refer to the covalent joining of two different molecules by a chemical reaction. The crosslinking can occur in one or more chemical reactions, as known in the art.

In the context of polypeptides, a “linear sequence” or a “sequence” is an order of amino acids in a polypeptide in an amino to carboxyl terminus (N- to C-terminus) direction in which residues that neighbor each other in the sequence are contiguous in the primary structure of the polypeptide. A “partial sequence” is a linear sequence of part of a polypeptide that is known to comprise additional residues in one or both directions.

“Heterologous” means derived from a genotypically distinct entity from the rest of the entity to which it is being compared. For example, a glycine rich sequence removed from its native coding sequence and operatively linked to a coding sequence other than the native sequence is a heterologous glycine rich sequence. The term “heterologous” as applied to a polynucleotide, a polypeptide, means that the polynucleotide or polypeptide is derived from a genotypically distinct entity from that of the rest of the entity to which it is being compared.

The terms “polynucleotides”, “nucleic acids”, “nucleotides” and “oligonucleotides” are used interchangeably. They refer to nucleotides of any length, encompassing a singular nucleic acid as well as plural nucleic acids, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, the term “reporter polypeptide(s)” refers to human polypeptide(s) or protein(s) that, under certain circumstances, can be acted upon to generate a detectable signal (such as being enzymatically digested to produce detectable peptide sequence(s)) that can be identified and characterized from outside of a cell, organ, tissue, or body of a subject. For example, a “reporter polypeptide” can be a human protein capable of being cleaved by protease(s) that are also capable of cleaving activatable therapeutic agent(s) (such as described hereinbelow) comprising peptide substrate. Non-limiting examples of peptide substrates include those described hereinbelow in section “Release Segments (RS).”

The term “complement of a polynucleotide” denotes a polynucleotide molecule having a complementary base sequence and reverse orientation as compared to a reference sequence, such that it could hybridize with a reference sequence with complete fidelity.

“Recombinant” as applied to a polynucleotide means that the polynucleotide is the product of various combinations of recombination steps which may include cloning, restriction and/or ligation steps, and other procedures that result in expression of a recombinant protein in a host cell.

The terms “gene” and “gene fragment” are used interchangeably herein. They refer to a polynucleotide containing at least one open reading frame that is capable of encoding a particular protein after being transcribed and translated. A gene or gene fragment may be genomic or cDNA, as long as the polynucleotide contains at least one open reading frame, which may cover the entire coding region or a segment thereof. A “fusion gene” is a gene composed of at least two heterologous polynucleotides that are linked together.

The term “homology” or “homologous” or “identity” interchangably refers to sequence similarity between two or more polynucleotide sequences or between two or more polypeptide sequences. When using a program such as BestFit to determine sequence identity, similarity or homology between two different amino acid sequences, the default settings may be used, or an appropriate scoring matrix, such as blosum45 or blosum80, may be selected to optimize identity, similarity or homology scores. Preferably, polynucleotides that are homologous are those which hybridize under stringent conditions as defined herein and have at least 70%, preferably at least 80%, more preferably at least 90%, more preferably 95%, more preferably 97%, more preferably 98%, and even more preferably 99% sequence identity, when optimally aligned, compared to those sequences. Polypeptides that are homologous preferably have sequence identities that are at least 70%, preferably at least 80%, even more preferably at least 90%, even more preferably at least 95-99% identical when optimally aligned over sequences of comparable length.

The terms “percent identity,” percentage of sequence identity,” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity may be measured over the length of an entire defined polynucleotide sequence, or may be measured over a shorter length, for example, over the length of a fragment taken from a larger, defined polynucleotide sequence, for instance, a fragment of at least 45, at least 60, at least 90, at least 120, at least 150, at least 210 or at least 450 contiguous residues. Such lengths are exemplary only, and it is understood that any fragment length supported by the sequences shown herein, in the tables, figures or Sequence Listing, may be used to describe a length over which percentage identity may be measured. The percentage of sequence identity is calculated by comparing two optimally aligned sequences over the window of comparison, determining the number of matched positions (at which identical residues occur in both polypeptide sequences), dividing the number of matched positions by the total number of positions in the window of comparison (e.g., the window size), and multiplying the result by 100 to yield the percentage of sequence identity. When sequences of different length are to be compared, the shortest sequence defines the length of the window of comparison. Conservative substitutions are not considered when calculating sequence identity.

“Percent (%) sequence identity” and “percent (%) identity” with respect to the polypeptide sequences identified herein, is defined as the percentage of amino acid residues in a query sequence that are identical with the amino acid residues of a second, reference polypeptide sequence of comparable length or a portion thereof, after aligning the sequences and introducing gaps, if necessary, to achieve the maximum percent sequence identity, and not considering any conservative substitutions as part of the sequence identity, thereby resulting in optimal alignment. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve optimal alignment over the full length of the sequences being compared. Percent identity may be measured over the length of an entire defined polypeptide sequence, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The term “expression” as used herein refers to a process by which a polynucleotide produces a gene product, for example, an RNA or a polypeptide. It includes without limitation transcription ofthe polynucleotide into messenger RNA (mRNA), transfer RNA (tRNA), small hairpin RNA (shRNA), small interfering RNA (siRNA) or any other RNA product, and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage.

A “vector” or “expression vector” are used interchangeably and refers to a nucleic acid molecule, preferably self-replicating in an appropriate host, which transfers an inserted nucleic acid molecule into and/or between host cells. The term includes vectors that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions. An “expression vector” is a polynucleotide which, when introduced into an appropriate host cell, can be transcribed and translated into a polypeptide(s). An “expression system” usually connotes a suitable host cell comprised of an expression vector that can function to yield a desired expression product.

The terms “t_{1/2}”, “half-life”, “terminal half-life”, “elimination half-life” and “circulating half-life” are used interchangeably herein and, as used herein, generally means the terminal half-life calculated as ln(2)/Kₑₗ. Kₑₗ is the terminal elimination rate constant calculated by linear regression of the terminal linear portion of the log concentration vs. time curve. Half-life typically refers to the time required for half the quantity of an administered substance deposited in a living organism to be metabolized or eliminated by normal biological processes. When a clearance curve of a given polypeptide is constructed as a function of time, the curve is usually biphasic with a rapid α-phase and longer beta-phase. The typical beta-phase half-life of a human antibody in humans is 21 days. Half-life can be measured using timed samples from any body fluid, but is most typically measured in serum or plasma samples.

The term “molecular weight” generally refers to the sum of atomic weights of the constituent atoms in a molecule. Molecular weight can be determined theoretically by summing the atomic masses of the constituent atoms in a molecule. When applied in the context of a polypeptide, the molecular weight is calculated by adding, based on amino acid composition, the molecular weight of each type of amino acid in the composition or by estimation from comparison to molecular weight standards in an SDS electrophoresis gel. The calculated molecular weight of a molecule can differ from the apparent molecular weight of a molecule, which generally refers to the molecular weight of a molecule as determined by one or more analytical techniques. “Apparent molecular weight factor” and “apparent molecular weight” are related terms and when used in the context of a polypeptide, the terms refer to a measure of the relative increase or decrease in apparent molecular weight exhibited by a particular amino acid or polypeptide sequence. The apparent molecular weight can be determined, for example, using size exclusion chromatography (SEC) or similar methods by comparing to globular protein standards, as measured in “apparent kD” units. The apparent molecular weight factor is the ratio between the apparent molecular weight and the “molecular weight”; the latter is calculated by adding, based on amino acid composition as described above, or by estimation from comparison to molecular weight standards in an SDS electrophoresis gel. The determination of apparent molecular weight and apparent molecular weight factor is described inter alia in U.S. Pat. No. 8,673,860.

The terms “hydrodynamic radius” or “Stokes radius” is the effective radius (Rh in nm) of a molecule in a solution measured by assuming that it is a body moving through the solution and resisted by the solution's viscosity. In the embodiments of the disclosure, the hydrodynamic radius measurements of the XTEN polypeptides correlate with the “apparent molecular weight factor” which is a more intuitive measure. The “hydrodynamic radius” of a protein affects its rate of diffusion in aqueous solution as well as its ability to migrate in gels of macromolecules. The hydrodynamic radius of a protein is determined by its molecular weight as well as by its structure, including shape and compactness. Methods for determining the hydrodynamic radius are well known in the art, such as by the use of size exclusion chromatography (SEC), as described inter alia in U.S. Pat. Nos. 6,406,632 and 7,294,513. Most proteins have globular structure, which is the most compact three-dimensional structure a protein can have with the smallest hydrodynamic radius. Some proteins adopt a random and open, unstructured, or ‘linear’ conformation and as a result have a much larger hydrodynamic radius compared to typical globular proteins of similar molecular weight.

“Physiological conditions” refers to a set of conditions in a living host as well as in vitro conditions, including temperature, salt concentration, pH, that mimic those conditions of a living subject. A host of physiologically relevant conditions for use in in vitro assays have been established. Generally, a physiological buffer contains a physiological concentration of salt and is adjusted to a neutral pH ranging from about 6.5 to about 7.8, and preferably from about 7.0 to about 7.5. A variety of physiological buffers are listed in Sambrook et al. (2001). Physiologically relevant temperature ranges from about 25° C. to about 38° C., and preferably from about 35° C. to about 37° C.

The term “binding moiety” is used herein in the broadest sense, and is specifically intended to include the categories of cytokines, cell receptors, antibodies or antibody fragments that have specific affinity for an antigen or ligand such as cell-surface receptors, target cell markers, or antigens or glycoproteins, oligonucleotides, enzymatic substrates, antigenic determinants, or binding sites that may be present in or on the surface of a tissue or cell.

The term “monoclonal antibody” as used herein refers to an antibody obtained from a population of substantially homogeneous antibodies, e.g., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being known in the art or described herein.

An “antibody fragment,” as used herein, generally refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)2, diabodies, single chain diabodies, linear antibodies, nanobodies (also known as single domain antibodies (including single domain camelid antibodies) or V_{HH}) single-chain variable fragment (scFv) antibody molecules, and multispecific antibodies formed from antibody fragments.

“scFv” or “single chain fragment variable” are used interchangeably herein to refer to an antibody fragment format comprising regions of variable heavy (“VH”) and variable light (“VL”) chains or two copies of a VH or VL chain, which are joined together by a short flexible peptide linker. The scFv is not actually a fragment of an antibody, but is a fusion protein of the variable regions of the heavy (VH) and light chains (VL) of immunoglobulins, and can be easily expressed in functional form in E. coli or mammalian cell(s) in either N- to C-terminus orientation; VL-VH or VH-VL.

The terms “antigen”, “target cell marker” and “ligand” are used interchangeably herein to refer to the structure or binding determinant that a binding moiety, an antibody, antibody fragment or an antibody fragment-based molecule binds to or has binding specificity against.

The term “epitope” refers to the particular site on an antigen molecule to which an antibody, antibody fragment, or binding moiety binds. An epitope is a ligand of an antibody, antibody fragment, or a binding moiety.

As used herein, “CD3” or “cluster of differentiation 3” means the T cell surface antigen CD3 complex, which includes in individual form or independently combined form all known CD3 subunits, for example CD3 epsilon, CD3 delta, CD3 gamma, CD3 zeta, CD3 alpha and CD3 beta. The extracellular domains of CD3 epsilon, gamma and delta contain an immunoglobulin-like domain, so are therefore considered part of the immunoglobulin superfamily.

The terms “specific binding” or “specifically bind” or “binding specificity” are used interchangeably herein to refer to the high degree of binding affinity of a binding moiety to its corresponding target. Typically, specific binding as measured by one or more of the assays disclosed herein would have a dissociation constant or K_{d} of less than about 10⁻⁶ M (e.g, of 10⁻⁷ M to 10⁻¹² M).

The term “affinity,” as used herein, generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{d}). As used herein “a greater binding affinity” or “increased binding affinity” means a lower K_{d} value; e.g., 1×10⁻⁹ M is a greater binding affinity than 1×10⁻⁸ M, while a “lower binding affinity” means a greater K_{d} value; e.g., 1×10⁻⁷ M is a lower binding affinity than 1×10⁻⁸ M.

“Inhibition constant”, or “Kᵢ”, are used interchangeably and mean the dissociation constant of the enzyme-inhibitor complex, or the reciprocal of the binding affinity of the inhibitor to the enzyme.

“Dissociation constant”, or “K_{d}”, are used interchangeably and mean the affinity between a ligand “L” and a protein “P”; e.g., how tightly a ligand binds to a particular protein. It can be calculated using the formula K_{d}=[L] [P]/[LP], where [P], [L] and [LP] represent molar concentrations of the protein, ligand and complex, respectively. The term “kₒₙ”, as used herein, is intended to refer to the on rate constant for association of an antibody to the antigen to form the antibody/antigen complex as is known in the art. The term “k_{off}”, as used herein, is intended to refer to the off rate constant for dissociation of an antibody from the antibody/antigen complex as is known in the art. Techniques such as flow cytometry or surface plasmon resonance can be used to detect binding events. The assays may comprise soluble antigens or receptor molecules, or may determine the binding to cell-expressed receptors. Such assays may include cell-based assays, including assays for proliferation, cell death, apoptosis and cell migration. The binding affinity of the subject compositions for the target ligands can be assayed using binding or competitive binding assays, such as Biacore assays with chip-bound receptors or binding proteins or ELISA assays, as described in U.S. Pat. No. 5,534,617, assays described in the Examples herein, radio-receptor assays, reporter gene activity assays, or other assays known in the art. For example, an exemplary reporter gene activity assay can be based on genetically engineered cell(s), generated by stably introducing relevant gene(s) for the receptor(s)-of-interest and the signaling pathway(s)-of-interest, such that binding to the engineered receptor triggers a signaling cascade leading to the activation of the engineered gene pathway with a subsequent production of signature polypeptide(s) (such as an enzyme). The binding affinity constant can then be determined using standard methods, such as Scatchard analysis, as described by van Zoelen, et al., Trends Pharmacol Sciences (1998) 19)12):487, or other methods known in the art.

A “target cell marker” refers to a molecule expressed by a target cell including but not limited to cell-surface receptors, cytokine receptors, antigens, tumor-associated antigens, glycoproteins, oligonucleotides, enzymatic substrates, antigenic determinants, or binding sites that may be present in the on the surface of a target tissue or cell that may serve as ligands for a binding moiety. Non-limiting examples of target cell markers include the target markers of Table 6.

The term “target tissue” generally refers to a tissue that is the cause of or is part of a disease condition such as, but not limited to cancer or inflammatory conditions. Sources of diseased target tissue include a body organ, a tumor, a cancerous cell or population of cancerous cells or cells that form a matrix or are found in association with a population of cancerous cells, bone, skin, cells that produce cytokines or factors contributing to a disease condition.

The term “target cell” generally refers to a cell that has the ligand of a binding moiety, an antibody or antibody fragment of the subject compositions and is associated with or causes a disease or pathologic condition, including cancer cells, tumor cells, and inflammatory cells. The ligand of a target cell is referred to herein as a “target cell marker” or “target cell antigen” and includes, but is not limited to, cell surface receptors or antigens, cytokines, cytokine receptors, MHC proteins, and cytosol proteins or peptides that are exogenously presented. As used herein, “target cell” would not include an effector cell.

As used herein, an “immunoassay” generally refers to a biochemical test that measures the presence or concentration of a substance in a sample, such as a biological sample, using the reaction of an antibody (or a fragment thereof) to its cognate antigen, for example the specific binding of an antibody to a protein. Both the presence of the antigen or the amount of the antigen present can be measured.

As used herein, a “mass spectrometer (MS)” generally refers to an apparatus that includes a means for ionizing molecules and detecting charged molecules. A mass spectrum generated by a mass spectrometer can be used to identify molecule(s) of interest based on the molar mass. Non-limiting examples of “mass spectrometer (MS)” include all combinations with liquid chromatography (LC), such as liquid chromatography with mass spectrometry (LC-MS), liquid chromatography with tandem mass spectrometry (LC-MS/MS), etc.

As used herein, the terms “treatment” or “treating,” or “palliating” or “ameliorating” are used interchangeably herein. These terms generally refer to an approach for obtaining beneficial or desired results including but not limited to a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms or improvement in one or more clinical parameters associated with the underlying disorder such that an improvement is observed in the subject, notwithstanding that the subject may still be afflicted with the underlying disorder. For prophylactic benefit, the compositions may be administered to a subject at risk of developing a particular disease, or to a subject reporting one or more of the physiological symptoms of a disease, even though a diagnosis of this disease may not have been made.

A “therapeutic effect” or “therapeutic benefit,” as used herein, generally refers to a physiologic effect, including but not limited to the mitigation, amelioration, or prevention of disease or an improvement in one or more clinical parameters associated with the underlying disorder in humans or other animals, or to otherwise enhance physical or mental wellbeing of humans or animals, resulting from administration of a polypeptide of the disclosure other than the ability to induce the production of an antibody against an antigenic epitope possessed by the biologically active protein. For prophylactic benefit, the compositions may be administered to a subject at risk of developing a particular disease, a recurrence of a former disease, condition or symptom of the disease, or to a subject reporting one or more of the physiological symptoms of a disease, even though a diagnosis of this disease may not have been made.

The terms “therapeutically effective amount” and “therapeutically effective dose”, as used herein, generally refer to an amount of a drug or a biologically active protein, either alone or as a part of a polypeptide composition, that is capable of having any detectable, beneficial effect on any symptom, aspect, measured parameter or characteristics of a disease state or condition when administered in one or repeated doses to a subject. Such effect need not be absolute to be beneficial. Determination of a therapeutically effective amount is well within the capability of those skilled in the art, especially in light of the detailed disclosure provided herein.

The term “equivalent molar dose” generally means that the amounts of materials administered to a subject have an equivalent amount of moles, based on the molecular weight of the material used in the dose.

The term “therapeutically effective and non-toxic dose,” as used herein, generally refers to a tolerable dose of the compositions as defined herein that is high enough to cause depletion of tumor or cancer cells, tumor elimination, tumor shrinkage or stabilization of disease without or essentially without major toxic effects in the subject. Such therapeutically effective and non-toxic doses may be determined by dose escalation studies described in the art and should be below the dose inducing severe adverse side effects.

The terms “cancer” and “cancerous” refer to or describe the physiological condition in mammals that is typically characterized by unregulated cell growth/proliferation.

### Compositions

### Therapeutic Agents

Provided herein, in some embodiments, is a therapeutic agent (or an activatable therapeutic agent, or a non-natural, activatable therapeutic agent) that comprises a release segment (RS) (such as one described hereinbelow in the RELEASE SEGMENTS section or described anywhere else herein) linked, directly or indirectly, to a biologically active moiety (BM) (such as one described hereinbelow in the BIOLOGICALLY ACTIVE MOIETIES section or described anywhere else herein). The biologically active moiety (BM) can be a biologically active peptide (BP) (such as one described hereinbelow in the BIOLOGICALLY ACTIVE MOIETIES section or described anywhere else herein). The release segment (RS) can comprise a peptide substrate (such as one described hereinbelow in the RELEASE SEGMENTS section or described anywhere else herein) susceptible to cleavage by a mammalian protease (such as one described hereinbelow or described anywhere else herein) at a scissile bond. The therapeutic agent can further comprise a masking moiety (MM) (such as one described hereinbelow in the MASKING MOIETIES section or described anywhere else herein) linked, directly or indirectly, to the release segment (RS). A bioactivity of the therapeutic agent can be enhanced upon cleavage of the peptide substrate by the mammalian protease (thereby releasing the masking moiety). The therapeutic agent, in an uncleaved state, can have a structural arrangement from N-terminus to C-terminus of BM-RS-MM or MM-RS-BM. Upon cleavage of the release segment (RS), the masking moiety (MM) can be released from the therapeutic agent. The masking moiety (MM) can comprise an extended recombinant polypeptide (XTEN). The therapeutic agent, in an uncleaved state, can have a structural arrangement from N-terminus to C-terminus of BM-RS-XTEN or XTEN-RS-BM.

In some embodiments of the therapeutic agent (or the activatable therapeutic agent, or the non-natural, activatable therapeutic agent), where the release segment (RS) can be a first release segment (RS1), where the peptide substrate (of the RS1) can be a first peptide substrate, and where the scissile bond (of the RS1) can be a first scissile bond, the therapeutic agent can further comprise a second release segment (RS2) (such as one described hereinbelow in the RELEASE SEGMENTS section or described anywhere else herein) linked, directly or indirectly, to the biologically active moiety (BM). The second release segment (RS2) can comprise a second peptide substrate (such as one described hereinbelow in the RELEASE SEGMENTS section or described anywhere else herein) for cleavage by a mammalian protease (such as one described hereinbelow or described anywhere else herein) at a second scissile bond. A bioactivity of the therapeutic agent can be enhanced upon cleavage of one or both of the first and second peptide substrate by the mammalian protease (thereby releasing one or both of the first and second masking moieties). The mammalian protease for cleavage of the second release segment (RS2) can be identical to the mammalian protease for cleavage of the first release segment (RS1). The mammalian protease for cleavage of the second release segment (RS2) can be different from the mammalian protease for cleavage of the first release segment (RS1). The second release segment (RS2) can have an amino acid sequence identical to that of the first release segment (RS1). The second release segment (RS2) can have an amino acid sequence different from that of the first release segment (RS1). In some embodiments, the scissile bond (or the first scissile bond, or the second scissile bond) is not immediately C-terminal to a methionine residue. In some embodiments, the first scissile bond is not immediately C-terminal to a methionine residue. In some embodiments, the second scissile bond is not immediately C-terminal to a methionine residue.

In some embodiments of the therapeutic agent (or the activatable therapeutic agent, or the non-natural, activatable therapeutic agent), where the masking moiety (MM) can be a first masking moiety (MM1), the therapeutic agent can further comprise a second masking moiety (MM2) (such as one described hereinbelow in the MASKING MOIETIES section or described anywhere else herein) linked, directly or indirectly, to the second release segment (RS2). The therapeutic agent, in an uncleaved state, can have a structural arrangement from N-terminus to C-terminus of MM1-RS1-BM-RS2-MM2, MM1-RS2-BM-RS1-MM2, MM2-RS1-BM-RS2-MM1, or MM2-RS2-BM-RS1-MM1. Upon cleavage of the second release segment (RS2), the second masking moiety (MM2) can be released from the therapeutic agent. The first masking moiety (MM1) can comprise a first extended recombinant polypeptide (XTEN1). The second masking moiety (MM2) can comprise a second extended recombinant polypeptide (XTEN2). The therapeutic agent, in an uncleaved state, can have a structural arrangement from N-terminus to C-terminus of XTEN1-RS1-BP-RS2-XTEN2, XTEN1-RS2-BP-RS1-XTEN2, XTEN2-RS1-BP-RS2-XTEN1, or XTEN2-RS2-BP-RS1-XTEN1.

In some embodiments of the therapeutic agent (or the activatable therapeutic agent, or the non-natural, activatable therapeutic agent), the therapeutic agent can comprise a fusion polypeptide (e.g., a recombinant fusion protein) or conjugate (e.g., linked by chemical conjugation). In some embodiments, the therapeutic agent can be configured for activation at or in proximity to a target tissue or cell (such as one described hereinbelow in the TARGET TISSUES OR CELLS section or described anywhere else herein) in a subject. The therapeutic agent can be an anti-cancer agent (such as an activatable anti-cancer agent, or a non-natural, activatable anti-cancer agent). The therapeutic agent can be configured for activation by one or more mammalian proteases (such as one or any combination of those described herein).

In some embodiments of the therapeutic agent (or the activatable therapeutic agent, or the non-natural, activatable therapeutic agent), the therapeutic agent can comprise a recombinant polypeptide. The recombinant polypeptide can comprise the biologically active peptide (BP) and the release segment (RS). The recombinant polypeptide can comprise the biologically active peptide (BP), the release segment (RS), and the masking moiety (MM). The recombinant polypeptide, in an uncleaved state, can have a structural arrangement from N-terminus to C-terminus of BP-RS-MM or MM-RS-BP. The recombinant polypeptide can comprise the biologically active peptide (BP), the first release segment (RS1), and the second release segment (RS2). The recombinant polypeptide can comprise the biologically active peptide (BP), the first release segment (RS1), the second release segment (RS2), the first masking moiety (MM1), and the second masking moiety (MM2). The recombinant polypeptide, in an uncleaved state, can have a structural arrangement from N-terminus to C-terminus of MM1-RS1-BP-RS2-MM2, MM1-RS2-BP-RS1-MM2, MM2-RS1-BP-RS2-MM1, or MM2-RS2-BP-RS1-MM1. The recombinant polypeptide can comprise the biologically active peptide (BP), the first release segment (RS1), the second release segment (RS2), the first extended recombinant polypeptide (XTEN1), and the second extended recombinant polypeptide (XTEN2). The recombinant polypeptide, in an uncleaved state, can have a structural arrangement from N-terminus to C-terminus of XTEN1-RS1-BP-RS2-XTEN2, XTEN1-RS2-BP-RS1-XTEN2, XTEN2-RS1-BP-RS2-XTEN1, or XTEN2-RS2-BP-RS1-XTEN1.

### Release Segments (RS)

In some embodiments of the therapeutic agent (or the activatable therapeutic agent, or the non-natural, activatable therapeutic agent), the release segment (RS) (or the first release segment (RS1), or the second release segment (RS2), can (each independently) comprise a peptide substrate susceptible to cleavage by a mammalian protease at a scissile bond. The release segment (RS) (or the first release segment (RS1), or the second release segment (RS2)) can (each independently) be cleaved when in proximity to a target tissue or cell (such as one described hereinbelow in the TARGET TISSUES OR CELLS section or described anywhere else herein), where the target tissue or cell can produce a mammalian protease (such as one described hereinbelow in the TARGET TISSUES OR CELLS section or described anywhere else herein) for which the release segment (RS) (or the first release segment (RS1), or the second release segment (RS2)) is a peptide substrate.

In some embodiments of the therapeutic agent (or the activatable therapeutic agent, or the non-natural, activatable therapeutic agent), the peptide substrate (or the first peptide substrate, or the second peptide substrate) can have at most four, or at most three, or at most two, or at most one amino acid substitution(s) with respect to a cleavage sequence (such as one set forth in Tables 1(a)-1(j) or Table A) of a reporter polypeptide (such as one described hereinbelow in the TARGET TISSUES OR CELLS section or described anywhere else herein). The peptide substrate (or the first peptide substrate, or the second peptide substrate) can have at most four, or at most three, or at most two, or at most one amino acid substitution(s) with respect to a cleavage sequence (such as one set forth in Tables 1(a)-1(j) or Table A) of the reporter polypeptide. The peptide substrate (or the first peptide substrate, or the second peptide substrate) can comprise an amino acid sequence identical to a cleavage sequence (such as one set forth in Tables 1(a)-1(j) or Table A) of the reporter polypeptide. In some embodiments of the therapeutic agent (or the activatable therapeutic agent, or the non-natural, activatable therapeutic agent), the peptide substrate (or the first peptide substrate, or the second peptide substrate) can comprise an amino acid sequence having at most four, or at most three, or at most two, or at most one amino acid substitution(s) with respect to a sequence set forth in Column II or III of Table A (or a subset thereof) and/or the group set forth in Tables 1(a)-1(j) (or any subset thereof). The peptide substrate (or the first peptide substrate, or the second peptide substrate) can comprise an amino acid sequence having at most four, or at most three, or at most two, or at most one amino acid substitution(s) with respect to a sequence set forth in Column II or III of Table A (or a subset thereof) and/or the group set forth in Tables 1(a)-1(j) (or any subset thereof). The peptide substrate (or the first peptide substrate, or the second peptide substrate) can comprise an amino acid sequence identical to a sequence set forth in Column II or III of Table A (or a subset thereof) and/or the group set forth in Tables 1(a)-1(j) (or any subset thereof). In some embodiments, the peptide substrate (or the first peptide substrate, or the second peptide substrate) comprises two or three sequences set forth in Column II or III of Table A (or a subset thereof). In some embodiments, where the peptide substrate (or the first peptide substrate, or the second peptide substrate) comprises two sequences set forth in Column II or III of Table A (or a subset thereof), the two sequences partially overlap one another. In some embodiments, where the peptide substrate (or the first peptide substrate, or the second peptide substrate) comprises two sequences set forth in Column II or III of Table A (or a subset thereof), the two sequences do not overlap one another. In some embodiments, where the peptide substrate (or the first peptide substrate, or the second peptide substrate) comprises three sequences set forth in Column II or III of Table A (or a subset thereof), two or all of the three sequences do not overlap one another. In some embodiments, where the peptide substrate (or the first peptide substrate, or the second peptide substrate) comprises three sequences set forth in Column II or III of Table A (or a subset thereof), one of the three sequences partially overlaps with another sequence or both other sequences of the three sequences. In some embodiments, where the peptide substrate (or the first peptide substrate, or the second peptide substrate) comprises three sequences set forth in Column II or III of Table A (or a subset thereof), two of the three sequences partially overlap with one another. In some embodiments, where the peptide substrate (or the first peptide substrate, or the second peptide substrate) comprises three sequences set forth in Column II or III of Table A (or a subset thereof), each two of the three sequences partially overlap with one another. In some embodiments, where the peptide substrate (or the first peptide substrate, or the second peptide substrate) comprises three sequences set forth in Column II or III of Table A (or a subset thereof), all of the three sequences partially overlap with one another. In some embodiments, none of the at most four, at most three, at most two, or at most one amino acid substitution(s) is/are at a position corresponding to an amino acid residue immediately adjacent to a scissile bond of a sequence set forth in Column II or III of Table A (or a subset thereof). In some embodiments, none of the at most four, at most three, at most two, or at most one amino acid substitution(s) is/are at a position corresponding to an amino acid residue immediately adjacent to a scissile bond of a corresponding sequence selected from the group set forth in Tables 1(a)-1(i) (or any subset thereof). In some embodiments, none of the at most four, at most three, at most two, or at most one amino acid substitution(s) is/are at a position corresponding to an amino acid residue immediately adjacent to a scissile bond of a corresponding sequence selected from the group set forth in Table 1(j) (or any subset thereof). The peptide substrate (or the first peptide substrate, or the second peptide substrate) can contain 4, 5, 6, 7, 8, 9, 10, 11, 12, 13, 14, 15, 16, 17, 18, 19, 20, 21, 22, 23, 24, or 25 amino acid residues or a range of any two of the foregoing values. The peptide substrate can contain from six to twenty-five or six to twenty amino acid residues. The peptide substrate can contain from six to twenty-five amino acid residues. The peptide substrate can contain from six to twenty amino acid residues. In some embodiments, the peptide substrate contains from seven to twelve amino acid residues. The peptide substrate can comprise a fragment of an amino acid sequence set forth in Column II or III of Table A (or a subset thereof) and/or the group set forth in Tables 1(a)-1(j) (or any subset thereof). The fragment of the peptide substrate can contain at least four amino acid residues and a corresponding scissile bond (such as indicated in Tables 1(a)-1(j) or Table A). The fragment of the peptide substrate can contain at least five, at least six, at least seven, at least eight, at least nine, or at least ten amino acid residues. In some cases, a portion of the peptide substrate that is N-terminal of the scissile bond can have at most four, or at most three, or at most two, or at most one amino acid substitution(s) with respect to a C-terminal end sequence containing from four to ten amino acid residues of a sequence set forth in Column IV or V of Table A (or a subset thereof). The portion of the peptide substrate that is N-terminal of the scissile bond can comprise a C-terminal end sequence containing from four to ten amino acid residues of a sequence set forth in Column IV or V of Table A (or a subset thereof). In some cases, a portion of the peptide substrate that is N-terminal of the scissile bond can have at most four, or at most three, or at most two, or at most one amino acid substitution(s) with respect to a C-terminal end sequence containing from four to ten amino acid residues of a sequence set forth in Column IV of Table A (or a subset thereof). The portion of the peptide substrate that is N-terminal of the scissile bond can comprise a C-terminal end sequence containing from four to ten amino acid residues of a sequence set forth in Column IV of Table A (or a subset thereof). In some cases, a portion of the peptide substrate that is N-terminal of the scissile bond can have at most four, or at most three, or at most two, or at most one amino acid substitution(s) with respect to a C-terminal end sequence containing from four to ten amino acid residues of a sequence set forth in Column V of Table A (or a subset thereof). The portion of the peptide substrate that is N-terminal of the scissile bond can comprise a C-terminal end sequence containing from four to ten amino acid residues of a sequence set forth in Column V of Table A (or a subset thereof). In some cases, a portion of the peptide substrate that is C-terminal of the scissile bond can have at most four, or at most three, or at most two, or at most one amino acid substitution(s) with respect to an N-terminal end sequence containing from four to ten amino acid residues of a sequence set forth in Column V or VI of Table A (or a subset thereof). The portion of the peptide substrate that is C-terminal of the scissile bond can an N-terminal end sequence containing from four to ten amino acid residues of a sequence set forth in Column V or VI of Table A (or a subset thereof). In some cases, a portion of the peptide substrate that is C-terminal of the scissile bond can have at most four, or at most three, or at most two, or at most one amino acid substitution(s) with respect to an N-terminal end sequence containing from four to ten amino acid residues of a sequence set forth in Column V of Table A (or a subset thereof). The portion of the peptide substrate that is C-terminal of the scissile bond can an N-terminal end sequence containing from four to ten amino acid residues of a sequence set forth in Column V of Table A (or a subset thereof). In some cases, a portion of the peptide substrate that is C-terminal of the scissile bond can have at most four, or at most three, or at most two, or at most one amino acid substitution(s) with respect to an N-terminal end sequence containing from four to ten amino acid residues of a sequence set forth in Column VI of Table A (or a subset thereof). The portion of the peptide substrate that is C-terminal of the scissile bond can an N-terminal end sequence containing from four to ten amino acid residues of a sequence set forth in Column VI of Table A (or a subset thereof). In some embodiments, where the peptide substrate comprises a scissile bond (for cleavage by one or more mammalian proteases), the peptide substrate does not comprise a methionine residue immediately N-terminal to the scissile bond. In some embodiments, where the peptide substrate comprises a plurality of scissile bonds, the peptide substrate does not comprise a methionine residue immediately N-terminal to at least one scissile bond of the plurality of scissile bonds. In some embodiments, where the peptide substrate comprises a plurality of scissile bonds, the peptide substrate does not comprise a methionine residue immediately N-terminal to each scissile bond of the plurality of scissile bonds. In some embodiments, the peptide substrate does not comprise an amino acid sequence selected from the group consisting of #279, #280, #282, #283, #298, #299, #302, #303, #305, #307, #308, #349, #396, #397, #416, #417, #418, #458, #459, #460, #466, #481 and #482 (or any combination thereof) of Column II of Table A.

In some embodiments of the therapeutic agent (or the activatable therapeutic agent, or the non-natural, activatable therapeutic agent) that comprises (1) a first release segment (RS1) comprising a first peptide substrate and (2) a second release segment (RS2) comprising a second peptide substrate, the second peptide substrate can contain 4, 5, 6, 7, 8, 9, 10, 11, 12, 13, 14, 15, 16, 17, 18, 19, 20, 21, 22, 23, 24, or 25 amino acid residues or a range of any two of the foregoing values. The second peptide substrate can contain from six to twenty-five or six to twenty amino acid residues. The second peptide substrate can contain from six to twenty-five amino acid residues. The second peptide substrate can contain from six to twenty amino acid residues. The second peptide substrate can contain from seven to twelve amino acid residues. The second peptide substrate can comprise an amino acid sequence having at most four, or at most three, or at most two, or at most one amino acid substitution(s) with respect to a sequence set forth in Column II or III of Table A (or a subset thereof) and/or the group set forth in Tables 1(a)-1(j) (or any subset thereof). The second peptide substrate can comprise an amino acid sequence having at most four, or at most three, or at most two, or at most one amino acid substitution(s) with respect to a sequence set forth in Column II or III of Table A (or a subset thereof) and/or the group set forth in Tables 1(a)-1(j) (or any subset thereof). The second peptide substrate can comprise an amino acid sequence identical to a sequence set forth in Column II or III of Table A (or a subset thereof) and/or the group set forth in Tables 1(a)-1(j) (or any subset thereof). In some embodiments, the second peptide substrate comprises two or three sequences set forth in Column II or III of Table A (or a subset thereof). In some embodiments, where the second peptide substrate comprises two sequences set forth in Column II or III of Table A (or a subset thereof), the two sequences (of the second peptide substrate) partially overlap one another. In some embodiments, where the second peptide substrate comprises two sequences set forth in Column II or III of Table A (or a subset thereof), the two sequences (of the second peptide substrate) do not overlap one another. In some embodiments, where the second peptide substrate comprises three sequences set forth in Column II or III of Table A (or a subset thereof), two or all of the three sequences (of the second peptide substrate) do not overlap one another. In some embodiments, where the second peptide substrate comprises three sequences set forth in Column II or III of Table A (or a subset thereof), one of the three sequences (of the second peptide substrate) partially overlaps with another sequence or both other sequences of the three sequences (of the second peptide substrate). In some embodiments, where the second peptide substrate comprises three sequences set forth in Column II or III of Table A (or a subset thereof), two of the three sequences (of the second peptide substrate) partially overlap with one another. In some embodiments, where the second peptide substrate comprises three sequences set forth in Column II or III of Table A (or a subset thereof), each two of the three sequences (of the second peptide substrate) partially overlap with one another. In some embodiments, where the second peptide substrate comprises three sequences set forth in Column II or III of Table A (or a subset thereof), all of the three sequences (of the second peptide substrate) partially overlap with one another. In some embodiments, where the second peptide substrate comprises a scissile bond (for cleavage by one or more mammalian proteases), the second peptide substrate does not comprise a methionine residue immediately N-terminal to the scissile bond. In some embodiments, where the second peptide substrate comprises a plurality of scissile bonds, the second peptide substrate does not comprise a methionine residue immediately N-terminal to at least one scissile bond of the plurality of scissile bonds. In some embodiments, where the second peptide substrate comprises a plurality of scissile bonds, the second peptide substrate does not comprise a methionine residue immediately N-terminal to each scissile bond of the plurality of scissile bonds. In some embodiments, the second peptide substrate does not comprise an amino acid sequence selected from the group consisting of #279, #280, #282, #283, #298, #299, #302, #303, #305, #307, #308, #349, #396, #397, #416, #417, #418, #458, #459, #460, #466, #481 and #482 (or any combination thereof) of Column II of Table A.

In some embodiments of the present disclosure, the peptide substrate (or the first peptide substrate, or the second peptide substrate) does not comprise a sequence selected from SEQ ID NOS: 1-8. In some embodiments, the peptide substrate (or the first peptide substrate, or the second peptide substrate) does not comprise a sequence of SEQ ID NO: 1. In some embodiments, the peptide substrate (or the first peptide substrate, or the second peptide substrate) does not comprise a sequence of SEQ ID NO: 2. In some embodiments, the peptide substrate (or the first peptide substrate, or the second peptide substrate) does not comprise a sequence of SEQ ID NO: 3. In some embodiments, the peptide substrate (or the first peptide substrate, or the second peptide substrate) does not comprise a sequence of SEQ ID NO: 4. In some embodiments, the peptide substrate (or the first peptide substrate, or the second peptide substrate) does not comprise a sequence of SEQ ID NO: 5. In some embodiments, the peptide substrate (or the first peptide substrate, or the second peptide substrate) does not comprise a sequence of SEQ ID NO: 6. In some embodiments, the peptide substrate (or the first peptide substrate, or the second peptide substrate) does not comprise a sequence of SEQ ID NO: 7. In some embodiments, the peptide substrate (or the first peptide substrate, or the second peptide substrate) does not comprise a sequence of SEQ ID NO: 8. In some embodiments, the peptide substrate (or the first peptide substrate, or the second peptide substrate) does not comprise a methionine residue immediately N-terminal to a scissile bond (contained therein) (for cleavage by one or more mammalian proteases). In some embodiments, the peptide substrate (or the first peptide substrate, or the second peptide substrate) does not comprise a methionine residue immediately N-terminal to one or more scissile bonds (contained therein). In some embodiments, the peptide substrate (or the first peptide substrate, or the second peptide substrate) does not comprise a methionine residue immediately N-terminal to any scissile bond (contained therein). In some embodiments, the peptide substrate (or the first peptide substrate or the second peptide substrate) does not comprise an amino acid sequence selected from the group consisting of #279, #280, #282, #283, #298, #299, #302, #303, #305, #307, #308, #349, #396, #397, #416, #417, #418, #458, #459, #460, #466, #481 and #482 (or any combination thereof) of Column II of Table A.

In some embodiments of the therapeutic agent (or the activatable therapeutic agent, or the non-natural, activatable therapeutic agent), a six to ten consecutive amino acid sequence of a peptide substrate (e.g., a first peptide substrate, a second peptide substrate, etc.) comprises at most four, at most three, at most two, or at most one amino acid substitution(s), with respect to a corresponding six to ten consecutive amino acid sequence of a sequence set forth in Column II or III of Table A (or a subset thereof). In some embodiments, a six to ten consecutive amino acid sequence of a peptide substrate (e.g., a first peptide substrate, a second peptide substrate, etc.) is identical to a corresponding six to ten consecutive amino acid sequence of a sequence set forth in Column II or III of Table A (or a subset thereof). In some embodiments, an eight to ten consecutive amino acid sequence of a peptide substrate (e.g., a first peptide substrate, a second peptide substrate, etc.) comprises at most three, at most two, or at most one amino acid substitution(s), with respect to a corresponding eight to ten consecutive amino acid sequence of a sequence set forth in Column II or III of Table A (or a subset thereof). In some embodiments, an eight to ten consecutive amino acid sequence of a peptide substrate (e.g., a first peptide substrate, a second peptide substrate, etc.) is identical to a corresponding eight to ten consecutive amino acid sequence of a sequence set forth in Column II or III of Table A (or a subset thereof). In some embodiments, an eight consecutive amino acid sequence of a peptide substrate (e.g., a first peptide substrate, a second peptide substrate, etc.) comprises at most three, at most two, or at most one amino acid substitution(s), with respect to a corresponding eight consecutive amino acid sequence of a sequence set forth in Column II or III of Table A (or a subset thereof). In some embodiments, an eight consecutive amino acid sequence of a peptide substrate (e.g., a first peptide substrate, a second peptide substrate, etc.) is identical to a corresponding eight consecutive amino acid sequence of a sequence set forth in Column II or III of Table A (or a subset thereof). In some embodiments, a nine consecutive amino acid sequence of a peptide substrate (e.g., a first peptide substrate, a second peptide substrate, etc.) comprises at most three, at most two, or at most one amino acid substitution(s), with respect to a corresponding nine consecutive amino acid sequence of a sequence set forth in Column II or III of Table A (or a subset thereof). In some embodiments, a nine consecutive amino acid sequence of a peptide substrate (e.g., a first peptide substrate, a second peptide substrate, etc.) is identical to a corresponding nine consecutive amino acid sequence of a sequence set forth in Column II or III of Table A (or a subset thereof). In some embodiments, a ten consecutive amino acid sequence of a peptide substrate (e.g., a first peptide substrate, a second peptide substrate, etc.) comprises at most three, at most two, or at most one amino acid substitution(s), with respect to a corresponding ten consecutive amino acid sequence of a sequence set forth in Column II or III of Table A (or a subset thereof). In some embodiments, a ten consecutive amino acid sequence of a peptide substrate (e.g., a first peptide substrate, a second peptide substrate, etc.) is identical to a corresponding ten consecutive amino acid sequence of a sequence set forth in Column II or III of Table A (or a subset thereof).

In some embodiments of the therapeutic agent (or the activatable therapeutic agent, or the non-natural, activatable therapeutic agent), the release segment (RS) (or the first release segment (RS1), or the second release segment (RS2), can (each independently) comprise a peptide substrate (or a first peptide substrate, or a second peptide substrate) for cleavage by a mammalian protease, such as a serine protease, a cysteine protease, an aspartate protease, a threonine protease, or a metalloproteinase. The release segment (RS) (or the first release segment (RS1), or the second release segment (RS2), can (independently) comprise a peptide substrate (or a first peptide substrate, or a second peptide substrate) for cleavage by a mammalian protease selected from the group consisting of disintegrin and metalloproteinase domain-containing protein 10 (ADAM10), disintegrin and metalloproteinase domain-containing protein 12 (ADAM12), disintegrin and metalloproteinase domain-containing protein 15 (ADAM15), disintegrin and metalloproteinase domain-containing protein 17 (ADAM17), disintegrin and metalloproteinase domain-containing protein 9 (ADAM9), disintegrin and metalloproteinase with thrombospondin motifs 5 (ADAMTS5), Cathepsin B, Cathepsin D, Cathepsin E, Cathepsin K, cathepsin L, cathepsin S, Fibroblast activation protein alpha, Hepsin, kallikrein-2, kallikrein-4, kallikrein-3, Prostate-specific antigen (PSA), kallikrein-13, Legumain, matrix metallopeptidase 1 (MMP-1), matrix metallopeptidase 10 (MMP-10), matrix metallopeptidase 11 (MMP-11), matrix metallopeptidase 12 (MMP-12), matrix metallopeptidase 13 (MMP-13), matrix metallopeptidase 14 (MMP-14), matrix metallopeptidase 16 (MMP-16), matrix metallopeptidase 2 (MMP-2), matrix metallopeptidase 3 (MMP-3), matrix metallopeptidase 7 (MMP-7), matrix metallopeptidase 8 (MMP-8), matrix metallopeptidase 9 (MMP-9), matrix metallopeptidase 4 (MMP-4), matrix metallopeptidase 5 (MMP-5), matrix metallopeptidase 6 (MMP-6), matrix metallopeptidase 15 (MMP-15), neutrophil elastase, protease activated receptor 2 (PAR2), plasmin, prostasin, PSMA-FOLH1, membrane type serine protease 1 (MT-SP1), matriptase, and u-plasminogen. The release segment (RS) (or the first release segment (RS1), or the second release segment (RS2), can (independently) comprise a peptide substrate (or a first peptide substrate, or a second peptide substrate) for cleavage by a mammalian protease selected from the group consisting of matrix metallopeptidase 1 (MMP1) (for which the sequences listed in Table 1(a), as examples without being limited to, are substrate sequences), matrix metallopeptidase 2 (MMP2) (for which the sequences listed in Table 1(b), as examples without being limited to, are substrate sequences), matrix metallopeptidase 7 (MMP1) (for which the sequences listed in Table 1(c), as examples without being limited to, are substrate sequences), matrix metallopeptidase 9 (MMP9) (for which the sequences listed in Table 1(d), as examples without being limited to, are substrate sequences), matrix metallopeptidase 11 (MMP11) (for which the sequences listed in Table 1(e), as examples without being limited to, are substrate sequences), matrix metallopeptidase 14 (MMP14) (for which the sequences listed in Table 1(f), as examples without being limited to, are substrate sequences), urokinase-type plasminogen activator (uPA) (for which the sequences listed in Table 1(g), as examples without being limited to, are substrate sequences), legumain (for which the sequences listed in Table 1(h), as examples without being limited to, are substrate sequences), and matriptase (for which the sequences listed in Table 1(i), as examples without being limited to, are substrate sequences). The release segment (RS) (or the first release segment (RS1), or the second release segment (RS2), can (independently) comprise a peptide substrate (or a first peptide substrate, or a second peptide substrate) for cleavage by a plurality of mammalian proteases. The peptide substrate (or the first peptide substrate, or the second peptide substrate) susceptible to cleavage by the mammalian protease can be susceptible to cleavage by a plurality of mammalian proteases comprising the mammalian protease. The peptide substrate (or the first peptide substrate, or the second peptide substrate) susceptible to cleavage by the plurality of mammalian proteases can have at most four, or at most three, or at most two, or at most one amino acid substitution(s) with respect to a sequence set forth in Table 1(j). The peptide substrate (or the first peptide substrate, or the second peptide substrate) susceptible to cleavage by the plurality of mammalian proteases can have at most four, or at most three, or at most two, or at most one amino acid substitution(s) with respect to a sequence set forth in Table 1(j). The peptide substrate (or the first peptide substrate, or the second peptide substrate) susceptible to cleavage by the plurality of mammalian proteases can have at most four, or at most three, or at most two, or at most one amino acid substitution(s) with respect to a sequence set forth in Table 1(j). The peptide substrate (or the first peptide substrate, or the second peptide substrate) susceptible to cleavage by the plurality of mammalian proteases can comprise a sequence set forth in Table 1(j).

In some embodiments of the therapeutic agent (or the activatable therapeutic agent, or the non-natural, activatable therapeutic agent) that comprises a set of release segments, each release segment in the set can (independently) comprise a peptide substrate for cleavage by a mammalian protease, such as a serine protease, a cysteine protease, an aspartate protease, a threonine protease, or a metalloproteinase. Each release segment in the set can (independently) comprise a peptide substrate for a different mammalian protease (independently) selected from the group consisting of disintegrin and metalloproteinase domain-containing protein 10 (ADAM10), disintegrin and metalloproteinase domain-containing protein 12 (ADAM12), disintegrin and metalloproteinase domain-containing protein 15 (ADAM15), disintegrin and metalloproteinase domain-containing protein 17 (ADAM17), disintegrin and metalloproteinase domain-containing protein 9 (ADAM9), disintegrin and metalloproteinase with thrombospondin motifs 5 (ADAMTS5), Cathepsin B, Cathepsin D, Cathepsin E, Cathepsin K, cathepsin L, cathepsin S, Fibroblast activation protein alpha, Hepsin, kallikrein-2, kallikrein-4, kallikrein-3, Prostate-specific antigen (PSA), kallikrein-13, Legumain, matrix metallopeptidase 1 (MMP-1), matrix metallopeptidase 10 (MMP-10), matrix metallopeptidase 11 (MMP-11), matrix metallopeptidase 12 (MMP-12), matrix metallopeptidase 13 (MMP-13), matrix metallopeptidase 14 (MMP-14), matrix metallopeptidase 16 (MMP-16), matrix metallopeptidase 2 (MMP-2), matrix metallopeptidase 3 (MMP-3), matrix metallopeptidase 7 (MMP-7), matrix metallopeptidase 8 (MMP-8), matrix metallopeptidase 9 (MMP-9), matrix metallopeptidase 4 (MMP-4), matrix metallopeptidase 5 (MMP-5), matrix metallopeptidase 6 (MMP-6), matrix metallopeptidase 15 (MMP-15), neutrophil elastase, protease activated receptor 2 (PAR2), plasmin, prostasin, PSMA-FOLH1, membrane type serine protease 1 (MT-SP1), matriptase, and u-plasminogen. Each release segment in the set can (independently) comprise a peptide substrate for a different mammalian protease (independently) selected from the group consisting of matrix metallopeptidase 1 (MMP1) (for which the sequences listed in Table 1(a), as examples without being limited to, are substrate sequences), matrix metallopeptidase 2 (MMP2) (for which the sequences listed in Table 1(b), as examples without being limited to, are substrate sequences), matrix metallopeptidase 7 (MMP1) (for which the sequences listed in Table 1(c), as examples without being limited to, are substrate sequences), matrix metallopeptidase 9 (MMP9) (for which the sequences listed in Table 1(d), as examples without being limited to, are substrate sequences), matrix metallopeptidase 11 (MMP11) (for which the sequences listed in Table 1(e), as examples without being limited to, are substrate sequences), matrix metallopeptidase 14 (MMP14) (for which the sequences listed in Table 1(f), as examples without being limited to, are substrate sequences), urokinase-type plasminogen activator (uPA) (for which the sequences listed in Table 1(g), as examples without being limited to, are substrate sequences), legumain (for which the sequences listed in Table 1(h), as examples without being limited to, are substrate sequences), and matriptase (for which the sequences listed in Table 1(i), as examples without being limited to, are substrate sequences). In some cases, at least one release segment (RS) of the set of release segments can (independently) comprise a peptide substrate for cleavage by a plurality of mammalian proteases. The peptide substrate susceptible to cleavage by the plurality of mammalian proteases can have at most four, or at most three, or at most two, or at most one amino acid substitution(s) with respect to a sequence set forth in Table 1(j). The peptide substrate susceptible to cleavage by the plurality of mammalian proteases can have at most four, or at most three, or at most two, or at most one amino acid substitution(s) with respect to a sequence set forth in Table 1(j). The peptide substrate susceptible to cleavage by the plurality of mammalian proteases can have at most four, or at most three, or at most two, or at most one amino acid substitution(s) with respect to a sequence set forth in Table 1(j). The peptide substrate susceptible to cleavage by the plurality of mammalian proteases can comprise a sequence set forth in Table 1(j). One of skill in the art will understand that a sequence set forth in Tables 1(a)-1(j) may, alternatively or additionally, be cleaved by one or more other proteases with substrate specificity similar to that of a corresponding protease, identified in a corresponding table, as capable of cleaving the sequence.

TABLE 1a
Exemplary peptide substrates for cleavage by
matrix metallopeptidase 1 (MMP1)

| | SEQ | |
|---|---|---|
| | ID | Amino Acid |
| Name of Reporter Polypeptide | NO: | Sequence |
| elastin | 36 | IGPGG-VAAAA |
| alpha-1-antitrypsin | 37 | DPQG-DAAQ |
| type I collagen alpha-1 chain | 38 | DGVRG-LTGPI |
| type V collagen alpha-1 chain | 39 | RGPSG-HMGRE |
| elastin | 40 | ISPEA-QAAAA |
| Complement C4-B OR Complement | 41 | TPLQ-LFEG |
| C4-A | | |
| type III collagen alpha-1 chain | 42 | QGPPG-KNGET |
| alpha-2-HS-glycoprotein | 43 | PPLG-APGL |
| apolipoprotein L1 | 44 | KPLG-DWAA |
| type II collagen alpha-1 chain | 45 | DGAAG-VKGDR |

TABLE 1b
Exemplary peptide substrates for cleavage by
matrix metallopeptidase 2 (MMP2)

| | SEQ | |
|---|---|---|
| | ID | Amino Acid |
| Name of Reporter Polypeptide | NO: | Sequence |
| alpha-1-antichymotrypsin | 46 | LLSA-LVET |
| pigment epithelium-derived factor | 47 | QPAH-LTFP |
| SPARC | 48 | DHPVE-LLARD |
| integrin alpha-IIb | 49 | QPSR-LQDP |
| type I collagen alpha-1 chain | 50 | DGVRG-LTGPI |
| zyxin | 51 | QPVS-LANT |
| elastin | 52 | IGPGG-VAAAA |
| vitronectin | 53 | LTSD-LQAQ |
| immunoglobulin kappa variable 2-30 | 54 | SPLS-LPVT |
| type IV collagen alpha-1 chain | 55 | GDPGE-ILGHV |

TABLE 1c
Exemplary peptide substrates for cleavage by
matrix metallopeptidase 7 (MMP7)

| | SEQ | |
|---|---|---|
| | ID | Amino Acid |
| Name of Reporter Polypeptide | NO: | Sequence |
| elastin | 56 | IGPGG-VAAAA |
| Complement C4-B OR Complement C4-A | 57 | TPLQ-LFEG |
| SPARC | 58 | DHPVE-LLARD |
| type I collagen alpha-1 chain | 59 | DGVRG-LTGPI |
| immunoglobulin kappa variable 2-30 | 60 | LPVT-LGQP |
| pigment epithelium-derived factor | 61 | QPAH-LTFP |
| probable non-functional | 62 | SPVT-LGQP |
| immunoglobulin kappa | | |
| variable 2D-24 | | |
| immunoglobulin kappa variable 3-20 | 63 | GTLS-LSPG |
| fibrinogen beta chain | 64 | EEAPS-LRPA |
| type II collagen alpha-1 chain | 65 | DGAAG-VKGDR |

TABLE 1(d)
Exemplary peptide substrates for cleavage by
matrix metallopeptidase 9 (MMP9)

| | SEQ | |
|---|---|---|
| | ID | Amino Acid |
| Name of Reporter Polypeptide | NO: | Sequence |
| type I collagen alpha-1 chain | 66 | DGVRG-LTGPI |
| elastin | 67 | IGPGG-VAAAA |
| type III collagen alpha-1 chain | 68 | QGPPG-KNGET |
| type V collagen alpha-1 chain | 69 | RGPSG-HMGRE |
| type II collagen alpha-1 chain | 70 | DGAAG-VKGDR |
| type VI collagen alpha-1 chain | 71 | KGAKG-YRGPE |
| alpha-2-HS-glycoprotein | 72 | PPLG-APGL |
| type VI collagen alpha-3 chain | 73 | IGNRG-PRGET |
| chromogranin-A | 74 | GPQL-RRGW |
| transcription factor SOX-10 | 75 | SPPG-VDAK |

TABLE 1(e)
Exemplary peptide substrates for cleavage by
matrix metallopeptidase 11 (MMP11)

| | SEQ | |
|---|---|---|
| | ID | Amino Acid |
| Name of Reporter Polypeptide | NO: | Sequence |
| alpha-1-antitrypsin | 76 | AAGA-MFLE |
| serum amyloid A-1 protein | 77 | AAEA-ISDA |
| fibrinogen alpha chain | 78 | EAAF-FDTA |
| complement C4-A OR complement | 79 | KSHA-LQLN |
| C4-B | | |
| apolipoprotein C-III | 80 | SARA-SEAE |
| ceruloplasmin | 81 | PAWA-KEKH |
| serum amyloid A-2 protein | 82 | AWAA-EVIS |
| fibrinogen beta chain | 83 | EEAPS-LRPA |
| immunoglobulin lambda variable | 84 | SEAS-YELT |
| 3-25 | | |
| PDZ and LIM domain protein 1 | 85 | PFTA-SPAS |

TABLE 1(f)
Exemplary peptide substrates for cleavage by
matrix metallopeptidase 14 (MMP14)

| | SEQ | |
|---|---|---|
| | ID | Amino Acid |
| Name of Reporter Polypeptide | NO: | Sequence |
| integrin alpha-IIb | 86 | QPSR-LQDP |
| alpha-1-antichymotrypsin | 87 | LLSA-LVET |
| pigment epithelium-derived factor | 88 | QPAH-LTFP |
| Complement C4-B OR Complement | 89 | TPLQ-LFEG |
| C4-A | | |
| zyxin | 90 | QPVS-LANT |
| type I collagen alpha-1 chain | 91 | DGVRG-LTGPI |
| SPARC | 92 | DHPVE-LLARD |
| immunoglobulin kappa variable | 93 | SPLS-LPVT |
| 2-30 | | |
| immunoglobulin kappa variable | 94 | LPVT-LGQP |
| 2-30 | | |
| elastin | 95 | IGPGG-VAAAA |

TABLE 1(g)
Exemplary peptide substrates for cleavage by
urokinase-type plasminogen activator (uPA)

| | SEQ | Amino |
|---|---|---|
| | ID | Acid |
| Name of Reporter Polypeptide | NO: | Sequence |
| serum amyloid A-2 protein | 96 | RSGR-DPNH |
| serum amyloid A-2 protein | 97 | AAKR-GPGG |
| deleted in malignant brain | 98 | RSKR-DVGS |
| tumors 1 protein | | |
| secretogranin-2 | 99 | VSKR-FPVG |
| serum amyloid A-1 protein OR | 100 | VSSR-SFFS |
| serum amyloid A-2 protein | | |
| haptoglobin | 101 | PVQR-ILGG |
| fibrinogen alpha chain | 102 | SSGP-GSTG |
| fibrinogen beta chain | 103 | FSAR-GHRP |
| complement C4-A OR complement | 104 | RQIR-GLEE |
| C4-B | | |
| oncoprotein-induced transcript | 105 | RMRR-GAGG |
| 3 protein | | |

TABLE 1(h)
Exemplary peptide substrates for cleavage by
legumain

| | SEQ | |
|---|---|---|
| | ID | Amino Acid |
| Name of Reporter Polypeptide | NO: | Sequence |
| neurosecretory protein VGF | 106 | RKKN-APPE |
| coagulation factor XII | 107 | GDRN-KPGV |
| Complement C4-B OR Complement | 108 | TGRN-GFKS |
| C4-A | | |
| fibrinogen alpha chain | 109 | GSWN-SGSS |
| tubulin beta chain | 110 | EPYN-ATLS |
| transthyretin | 111 | FTAN-DSGP |
| fibrinogen beta chain | 112 | QGVN-DNEE |
| fibrinogen alpha chain | 113 | SPRN-PSSA |
| angiotensinogen | 114 | QQLN-KPEV |
| multimerin-1 | 115 | TSLN-TVGG |

TABLE 1(i)
Exemplary peptide substrates for cleavage by
matriptase

| | SEQ | Amino |
|---|---|---|
| | ID | Acid |
| Name of Reporter Polypeptide | NO: | Sequence |
| oncoprotein-induced transcript | 116 | RMRR-GAGG |
| 3 protein | | |
| deleted in malignant brain | 117 | RSKR-DVGS |
| tumors 1 protein | | |
| serum amyloid A-2 protein | 118 | AAKR-GPGG |
| inter-alpha-trypsin inhibitor | 119 | RVPR-QVRL |
| heavy chain H5 | | |
| haptoglobin | 120 | PVQR-ILGG |
| alpha-2-HS-glycoprotein | 121 | RKTR-TVVQ |
| sulfhydryl oxidase 1 | 122 | PGLR-AAPG |
| gastric inhibitory polypeptide | 123 | RGPR-YAEG |
| keratin, type I cytoskeletal 17 | 124 | RQVR-TIVE |
| complement C4-A OR complement | 125 | RQIR-GLEE |
| C4-B | | |

TABLE 1(j)
Exemplary peptide substrates for cleavage by
multiple proteases

| SEQ | | Exemplary Proteases That |
|---|---|---|
| ID | | May Cleave the Peptide |
| NO. | Amino Acid Sequence | substrate |
| 1 | GPGG-VAAAVSKR-FPVG | MMP2, MMP7, uPA |
| 2 | GVRG-LTGPVSKR-FPVG | MMP2, MMP7, uPA |
| 3 | VSKR-FPVGEAGR-SAN-H | uPA, matriptase, |
| | | legumain |
| 4 | EAGR-SAN-HGVRG-LTGP | matriptase, legumain, |
| | | MMP1 |
| 5 | EAGR-SAN-HTPAG-LTGP | MMP2, MMP9, matriptase, |
| | | legumain |
| 6 | SPEA-QAAAEAGR-SAN-H | MMP1, matriptase, |
| | | legumain |
| 7 | QPAH-LTFPEAGR-SAN-H | MMP2, MMP14, legumain, |
| | | matriptase |
| 8 | AGSPGK-DGVRG-LTGP | matriptase, MMP2, MMP9 |

### Masking Moieties (MM)

A masking moiety (MM) of the present disclosure may be capable of specifically or non-specifically interacting with a biologically active moiety (BM) (or any component(s) or fragment(s) thereof) of an activatable therapeutic agent composition (such as described herein), thereby masking the BM (at least in certain cases) by inhibiting or reducing the ability of the BM to bind with designated target(s). In some instances, the masking moiety (MM) may specifically bind to or have specific affinity for the biologically active moiety (e.g., an antibody or antibody fragment), thereby interfering and/or inhibiting binding of the BM to its designed target (e.g., antigen target). In some instances, the masking moiety does not have significant affinity for the biologically active moiety, but exerts it masking effect due to non-specific steric hinderance.

In some embodiments of the therapeutic agent (or the activatable therapeutic agent, or the non-natural, activatable therapeutic agent), the masking moiety (MM) (or the first masking moiety (MM1), or the second masking moiety (MM2)), when linked to the corresponding therapeutic agent, can (each independently, individually or collectively) interfere with an interaction of the biologically active moiety (BM) to a target tissue or cell (such as one described hereinbelow in the TARGET TISSUES OR CELLS section or described anywhere else herein) such that a dissociation constant (K_{d}) of the BM of the therapeutic agent with a target cell marker (such as one described hereinbelow in the TARGET TISSUES OR CELLS section or described anywhere else herein) borne by the target tissue or cell can be greater, when the therapeutic agent is in an uncleaved state, compared to a dissociation constant (K_{d}) of a corresponding biologically active moiety (as remaining after the release segment (RS) is cleaved and the MM is released) with the target cell marker. The dissociation constant (K_{d}) of the biologically active moiety (BM) of the therapeutic agent, when the therapeutic agent is in an uncleaved state, with the target cell marker can be at least (about) 2-fold greater, at least (about) 5-fold greater, at least (about) 10-fold greater, at least (about) 50-fold greater, at least (about) 100-fold greater, at least (about) 200-fold greater, at least (about) 300-fold greater, at least (about) 400-fold greater, at least (about) 500-fold greater, at least (about) 600-fold greater, at least (about) 700-fold greater, at least (about) 800-fold greater, at least (about) 900-fold greater, or at least (about) 1000-fold greater, than the dissociation constant (Kd) of the corresponding biologically active moiety with the target cell marker. The dissociation constant (Kd) can be measured in an in vitro assay under equivalent molar concentrations. The in vitro assay can be selected from cell membrane integrity assay, mixed cell culture assay, cell-based competitive binding assay, FACS based propidium Iodide assay, trypan Blue influx assay, photometric enzyme release assay, radiometric 51Cr release assay, fluorometric Europium release assay, CalceinAM release assay, photometric MTT assay, XTT assay, WST-1 assay, alamar blue assay, radiometric 3H-Thd incorporation assay, clonogenic assay measuring cell division activity, fluorometric rhodamine123 assay measuring mitochondrial transmembrane gradient, apoptosis assay monitored by FACS-based phosphatidylserine exposure, ELISA-based TUNEL test assay, sandwich ELISA, caspase activity assay, cell-based LDH release assay, and cell morphology assay, reporter gene activity assay, or any combination thereof.

In some embodiments of the therapeutic agent (or the activatable therapeutic agent, or the non-natural, activatable therapeutic agent), the therapeutic agent can effect an enhancement in a safety profile, for example, improve a maximum tolerable exposure level (MTEL), and/or reduce a side effect (e.g., cytotoxicity), in delivery of the BM to a target tissue or cell (such as one described hereinbelow in the TARGET TISSUES OR CELLS section or described anywhere else herein) compared to a corresponding biologically active moiety (as remaining after the release segment (RS) is cleaved and the MM is released). The therapeutic agent, in which the biologically active moiety (BM) is linked (directly or indirectly) to the masking moiety (MM) (or the first masking moiety (MM1), or the second masking moiety (MM2)) can effect an enhancement in a safety profile, for example, improve a maximum tolerable exposure level (MTEL), and/or reduce a side effect (e.g., cytotoxicity), by at least (about) 2-fold, by at least (about) 5-fold, by at least (about) 10 fold, by at least (about) 50-fold, by at least (about) 100-fold, by at least (about) 200-fold, by at least (about) 300-fold, by at least (about) 400-fold, or by at least (about) 500-fold higher, in delivery of the BM to the target tissue or cell, than the corresponding biologically active moiety.

In some embodiments of the therapeutic agent (or the activatable therapeutic agent, or the non-natural, activatable therapeutic agent), the therapeutic agent can have a longer terminal half-life compared to that of a corresponding biologically active moiety. The therapeutic agent, in which the biologically active moiety (BM) is linked (directly or indirectly) to the masking moiety (MM) (or the first masking moiety (MM1), or the second masking moiety (MM2)) can have a terminal half-life of at least (about) 2-fold longer, at least (about) 5-fold longer, at least (about) 10-fold longer, at least (about) 15-fold longer, at least (about) 20-fold longer, at least (about) 50-fold longer, or at least (about) 100-fold longer, than the terminal half-life of the corresponding biologically active moiety.

In some embodiments, the therapeutic agent can be less immunogenic compared to a corresponding biologically active moiety. The therapeutic agent, in which the biologically active moiety (BM) is linked (directly or indirectly) to the masking moiety (MM) (or the first masking moiety (MM1), or the second masking moiety (MM2)), can be at least (about) 2-fold less immunogenic, at least (about) 5-fold less immunogenic, or at least (about) 10-fold less immunogenic, than the corresponding biologically active moiety. The immunogenicity can be ascertained by measuring production of IgG antibodies that selectively bind to the biologically active moiety after administration of comparable doses to a subject.

In some embodiments, the therapeutic agent can have a greater apparent molecular weight factor under a physiological condition, compared to a corresponding biologically active moiety. The therapeutic agent, in which the biologically active moiety (BM) is linked (directly or indirectly) to the masking moiety (MM) (or the first masking moiety (MM1), or the second masking moiety (MM2)), can have an apparent molecular weight factor of at least (about) 1.5-fold greater, at least (about) 2-fold greater, at least (about) 5-fold greater, at least (about) 8-fold greater, at least (about) 10-fold greater, at least (about) 12-fold greater, at least (about) 15-fold greater, at least (about) 18-fold greater, or at least (about) 20-fold greater, under a physiological condition, than the corresponding biologically active moiety.

In some embodiments of the therapeutic agent (or the activatable therapeutic agent, or the non-natural, activatable therapeutic agent) that comprises a first masking moiety (MM1) and a second masking moiety (MM2), the MM1 and the MM2, when both linked in the therapeutic agent, can (each independently, individually or collectively) interfere with an interaction of the biologically active moiety (BM) to a target tissue or cell (such as one described hereinbelow in the TARGET TISSUES OR CELLS section or described anywhere else herein) such that a dissociation constant (K_{d}) of the biologically active moiety (BM) of the therapeutic agent with a target cell marker (such as one described hereinbelow in the TARGET TISSUES OR CELLS section or described anywhere else herein) borne by the target tissue or cell can be greater, when the therapeutic agent is in an uncleaved state, compared to a dissociation constant (Kd) of a corresponding biologically active peptide (as remaining after one or both of the first release segment (RS1) and the second release segment (RS2) is/are cleaved and one or both of the MM1 and the MM2 is/are released). The dissociation constant (Kd) of the biologically active moiety (BM) of the therapeutic agent, when the therapeutic agent is in an uncleaved state, with the target cell marker can be at least (about) 2-fold greater, at least (about) 5-fold greater, at least (about) 10-fold greater, at least (about) 50-fold greater, at least (about) 100-fold greater, at least (about) 200-fold greater, at least (about) 300-fold greater, at least (about) 400-fold greater, at least (about) 500-fold greater, at least (about) 600-fold greater, at least (about) 700-fold greater, at least (about) 800-fold greater, at least (about) 900-fold greater, or at least (about) 1000-fold greater, than the dissociation constant (Kd) of the corresponding biologically active peptide. The dissociation constant (Kd) can be measured in an in vitro assay under equivalent molar concentrations. The in vitro assay can be selected from cell membrane integrity assay, mixed cell culture assay, cell-based competitive binding assay, FACS based propidium Iodide assay, trypan Blue influx assay, photometric enzyme release assay, radiometric ⁵¹Cr release assay, fluorometric Europium release assay, CalceinAM release assay, photometric MTT assay, XTT assay, WST-1 assay, alamar blue assay, radiometric 3H-Thd incorporation assay, clonogenic assay measuring cell division activity, fluorometric rhodamine123 assay measuring mitochondrial transmembrane gradient, apoptosis assay monitored by FACS-based phosphatidylserine exposure, ELISA-based TUNEL test assay, sandwich ELISA, caspase activity assay, cell-based LDH release assay, reporter gene activity assay, and cell morphology assay, or any combination thereof.

In some embodiments of the therapeutic agent (or the activatable therapeutic agent, or the non-natural, activatable therapeutic agent) that comprises a first masking moiety (MM1) and a second masking moiety (MM2), the therapeutic agent, in which the biologically active moiety (BM) is linked, directly or indirectly, to one or both of the MM1 and the MM2, can effect an enhancement in a safety profile, for example, improve a maximum tolerable exposure level (MTEL), and/or reduce a side effect (e.g., cytotoxicity), in delivery of the biologically active moiety (BM) to the target tissue or cell compared to a corresponding biologically active moiety (as remaining after one or both of the first release segment (RS1) and the second release segment (RS2) is/are cleaved and one or both of the MM1 and the MM2 is/are released). The therapeutic agent, in which the biologically active moiety (BM) is linked (directly or indirectly) to one or both of the MM1 and the MM2, can effect an enhancement in a safety profile, for example, improve a maximum tolerable exposure level (MTEL), and/or reduce a side effect (e.g., cytotoxicity) by at least (about) 2-fold, by at least (about) 5-fold, by at least (about) 10 fold, by at least (about) 50-fold, by at least (about) 100-fold, by at least (about) 200-fold, by at least (about) 300-fold, by at least (about) 400-fold, or by at least (about) 500-fold higher in delivery of the BM to the target tissue or cell, than the corresponding biologically active moiety.

In some embodiments of the therapeutic agent (or the activatable therapeutic agent, or the non-natural, activatable therapeutic agent) that comprises a first masking moiety (MM1) and a second masking moiety (MM2), the therapeutic agent, in which the biologically active moiety (BM) is linked, directly or indirectly, to one or both of the MM1 and the MM2, can have a longer terminal half-life compared to that of a corresponding biologically active moiety (as remaining after one or both of the first release segment (RS1) and the second release segment (RS2) is/are cleaved and one or both of the MM1 and the MM2 is/are released). The therapeutic agent, in which the biologically active moiety (BM) is linked (directly or indirectly) to one or both of the MM1 and the MM2, can have a terminal half-life of at least (about) 2-fold longer, at least (about) 5-fold longer, at least (about) 10-fold longer, at least (about) 15-fold longer, at least (about) 20-fold longer, at least (about) 50-fold longer, at least (about) 100-fold longer, than the terminal half-life of the corresponding biologically active moiety.

In some embodiments of the therapeutic agent (or the activatable therapeutic agent, or the non-natural, activatable therapeutic agent) that comprises a first masking moiety (MM1) and a second masking moiety (MM2), the therapeutic agent, in which the biologically active moiety (BM) is linked, directly or indirectly, to one or both of the MM1 and MM2, can be less immunogenic compared to a corresponding biologically active moiety (as remaining after one or both of the first release segment (RS1) and the second release segment (RS2) is/are cleaved and one or both of the MM1 and the MM2 is/are released). The therapeutic agent, in which the biologically active moiety (BM) is linked (directly or indirectly) to one or both of the MM1 and the MM2, can be at least (about) 2-fold less immunogenic, at least (about) 5-fold less immunogenic, or at least (about) 10-fold less immunogenic, than the corresponding biologically active moiety. The immunogenicity can be ascertained by measuring production of IgG antibodies that selectively bind to the biologically active moiety after administration of comparable doses to a subject.

In some embodiments of the therapeutic agent (or the activatable therapeutic agent, or the non-natural, activatable therapeutic agent) that comprises a first masking moiety (MM1) and a second masking moiety (MM2), the therapeutic agent, in which the biologically active moiety (BM) is linked, directly or indirectly, to one or both of the MM1 and the MM2, can have a greater apparent molecular weight factor under a physiological condition compared to a corresponding biologically active moiety. The therapeutic agent, in which the biologically active moiety (BM) is linked (directly or indirectly) to one or both of the MM1 and the MM2, can have an apparent molecular weight factor of at least (about) 1.5-fold greater, at least (about) 2-fold greater, at least (about) 5-fold greater, at least (about) 8-fold greater, at least (about) 10-fold greater, at least (about) 12-fold greater, at least (about) 15-fold greater, at least (about) 18-fold greater, or at least (about) 20-fold greater, under a physiological condition, than the corresponding biologically active moiety.

In some embodiments of the therapeutic agent (or the activatable therapeutic agent, or the non-natural, activatable therapeutic agent), the masking moiety (MM) (or the first masking moiety (MM1), or the second masking moiety (MM2)) can (each independently) comprise an extended recombinant polypeptide (XTEN). The XTEN can be characterized in that: (i) it comprises at least 100 amino acids; (ii) at least 90% of the amino acid residues of it are selected from glycine (G), alanine (A), serine (S), threonine (T), glutamate (E) and proline (P); and (iii) it comprises at least 4 different types of amino acids selected from G, A, S, T, E, and P. The XTEN can be characterized in that: (i) it comprises at least 150 amino acids; (ii) at least 90% of the amino acid residues of it are selected from glycine (G), alanine (A), serine (S), threonine (T), glutamate (E) and proline (P); and (iii) it comprises at least 4 different types of amino acids selected from G, A, S, T, E, and P. The extended recombinant polypeptide (XTEN) can (each independently) comprise an amino acid sequence having at least (about) 90%, at least (about) 91%, at least (about) 92%, at least (about) 93%, at least (about) 94%, at least (about) 95%, at least (about) 96%, at least (about) 97%, at least (about) 98%, at least (about) 99%, or 100% sequence identity to a sequence set forth in Tables 2b-2c, or any subset thereof.

In some embodiments of the therapeutic agent (or the activatable therapeutic agent, or the non-natural, activatable therapeutic agent) that comprises (1) a first masking moiety (MM1) comprising a first extended recombinant polypeptide (XTEN1) and (2) a second masking moiety (MM2) comprising a second extended recombinant polypeptide (XTEN2), the XTEN2 can be characterized in that: (i) it comprises at least 100 amino acids; (ii) at least 90% of the amino acid residues of it are selected from glycine (G), alanine (A), serine (S), threonine (T), glutamate (E) and proline (P); and (iii) it comprises at least 4 different types of amino acids selected from G, A, S, T, E, and P. The XTEN2 can be characterized in that: (i) it comprises at least 150 amino acids; (ii) at least 90% of the amino acid residues of it are selected from glycine (G), alanine (A), serine (S), threonine (T), glutamate (E) and proline (P); and (iii) it comprises at least 4 different types of amino acids selected from G, A, S, T, E, and P. The XTEN2 can comprise an amino acid sequence having at least (about) 90%, at least (about) 91%, at least (about) 92%, at least (about) 93%, at least (about) 94%, at least (about) 95%, at least (about) 96%, at least (about) 97%, at least (about) 98%, at least (about) 99%, or 100% sequence identity to a sequence selected from the group of sequences set forth in Tables 2b-2c, or any subset thereof.

In some embodiments, the XTEN (or the XTEN1, or the XTEN2) can (each independently) comprise, or can (each independently) be formed from, a plurality of non-overlapping sequence motifs. At least one of the non-overlapping sequence motifs can be recurring (or repeated at least two times in the corresponding XTEN). At least one of the non-overlapping sequence motifs can be non-recurring (or found only once within the corresponding XTEN). The plurality of non-overlapping sequence motifs can comprise (i) a set of (recurring) non-overlapping sequence motifs, where each motif of the set is repeated at least two times in the corresponding XTEN and (ii) a non-overlapping (non-recurring) sequence motif that occurs (or is found) only once within the corresponding XTEN. Each non-overlapping sequence motif can be from 9 to 14 (or 10 to 14, or 11 to 13) amino acids in length. Each non-overlapping sequence motif can be 12 amino acids in length. The plurality of non-overlapping sequence motifs can comprise a set of non-overlapping (recurring) sequence motifs, where each motif of the set can be (1) repeated at least two times in the corresponding XTEN and (2) between 9 and 14 amino acids in length. The set of (recurring) non-overlapping sequence motifs can comprise 12-mer sequence motifs selected from the group set forth in Table 2a. The set of (recurring) non-overlapping sequence motifs can comprise 12-mer sequence motifs selected from the group set forth in Table 2a. The set of (recurring) non-overlapping sequence motifs can comprise at least two, at least three, or all four of 12-mer sequence motifs of the group set forth in Table 2a.

TABLE 2a
Exemplary 12-mer sequence motifs for construction
of the XTENs

| | SEQ | |
|---|---|---|
| | ID | |
| Motif Family* | NO: | Amino Acid Sequence |
| AD | 126 | GESPGGSSGSES |
| AD | 127 | GSEGSSGPGESS |
| AD | 128 | GSSESGSSEGGP |
| AD | 129 | GSGGEPSESGSS |
| AE, AM | 130 | GSPAGSPTSTEE |
| AE, AM, AQ | 131 | GSEPATSGSETP |
| AE, AM, AQ | 132 | GTSESATPESGP |
| AE, AM, AQ | 133 | GTSTEPSEGSAP |
| AF, AM | 134 | GSTSESPSGTAP |
| AF, AM | 135 | GTSTPESGSASP |
| AF, AM | 136 | GTSPSGESSTAP |
| AF, AM | 137 | GSTSSTAESPGP |
| AG, AM | 138 | GTPGSGTASSSP |
| AG, AM | 139 | GSSTPSGATGSP |
| AG, AM | 140 | GSSPSASTGTGP |
| AG, AM | 141 | GASPGTSSTGSP |
| AQ | 142 | GEPAGSPTSTSE |
| AQ | 143 | GTGEPSSTPASE |
| AQ | 144 | GSGPSTESAPTE |
| AQ | 145 | GSETPSGPSETA |
| AQ | 146 | GPSETSTSEPGA |
| AQ | 147 | GSPSEPTEGTSA |
| BC | 148 | GSGASEPTSTEP |
| BC | 149 | GSEPATSGTEPS |
| BC | 150 | GTSEPSTSEPGA |
| BC | 151 | GTSTEPSEPGSA |
| BD | 152 | GSTAGSETSTEA |
| BD | 153 | GSETATSGSETA |
| BD | 154 | GTSESATSESGA |
| BD | 155 | GTSTEASEGSAS |

*Denotes individual motif sequences that, when used together in various permutations, results in a “family sequence”

TABLE 2b
Exemplary XTEN polypeptides

| XTEN | SEQ ID | |
|---|---|---|
| Name | NO. | Amino Acid Sequence |
| AE144 | 156 | GSEPATSGSETPGTSESATPESGPGSEPATSGSETPGSPAGSPTSTEEGTST |
| | | EPSEGSAPGSEPATSGSETPGSEPATSGSETPGSEPATSGSETPGTSTEPSE |
| | | GSAPGTSESATPESGPGSEPATSGSETPGTSTEPSEGSAP |
| AE144_1A | 157 | SPAGSPTSTEEGTSESATPESGPGTSTEPSEGSAPGSPAGSPTSTEEGTSTE |
| | | PSEGSAPGTSTEPSEGSAPGTSESATPESGPGSEPATSGSETPGSEPATSGS |
| | | ETPGSPAGSPTSTEEGTSESATPESGPGTSTEPSEGSAPG |
| AE144_2A | 158 | TSTEPSEGSAPGSPAGSPTSTEEGTSTEPSEGSAPGTSTEPSEGSAPGTSES |
| | | ATPESGPGTSTEPSEGSAPGTSESATPESGPGSEPATSGSETPGTSTEPSEG |
| | | SAPGTSTEPSEGSAPGTSESATPESGPGTSESATPESGPG |
| AE144_2B | 159 | TSTEPSEGSAPGSPAGSPTSTEEGTSTEPSEGSAPGTSTEPSEGSAPGTSES |
| | | ATPESGPGTSTEPSEGSAPGTSESATPESGPGSEPATSGSETPGTSTEPSEG |
| | | SAPGTSTEPSEGSAPGTS ESATPESGPGTSESATPESGPG |
| AE144_3A | 160 | SPAGSPTSTEEGTSESATPESGPGSEPATSGSETPGTSESATPESGPGTSTE |
| | | PSEGSAPGTSTEPSEGSAPGTSTEPSEGSAPGTSTEPSEGSAPGTSTEPSEG |
| | | SAPGTSTEPSEGSAPGSPAGSPTSTEEGTSTEPSEGSAPG |
| AE144_3B | 161 | SPAGSPTSTEEGTSESATPESGPGSEPATSGSETPGTSESATPESGPGTSTE |
| | | PSEGSAPGTSTEPSEGSAPGTSTEPSEGSAPGTSTEPSEGSAPGTSTEPSEG |
| | | SAPGTSTEPSEGSAPGSPAGSPTSTEEGTSTEPSEGSAPG |
| AE144_4A | 162 | TSESATPESGPGSEPATSGSETPGTSESATPESGPGSEPATSGSETPGTSES |
| | | ATPESGPGTSTEPSEGSAPGTSESATPESGPGSPAGSPTSTEEGSPAGSPTS |
| | | TEEGSPAGSPTSTEEGTSESATPESGPGTSTEPSEGSAPG |
| AE144_4B | 163 | TSESATPESGPGSEPATSGSETPGTSESATPESGPGSEPATSGSETPGTSES |
| | | ATPESGPGTSTEPSEGSAPGTSESATPESGPGSPAGSPTSTEEGSPAGSPTS |
| | | TEEGSPAGSPTSTEEGTSESATPESGPGTSTEPSEGSAPG |
| AE144_5A | 164 | TSESATPESGPGSEPATSGSETPGTSESATPESGPGSEPATSGSETPGTSES |
| | | ATPESGPGTSTEPSEGSAPGSPAGSPTSTEEGTSESATPESGPGSEPATSGS |
| | | ETPGTSESATPESGPGSPAGSPTSTEEGSPAGSPTSTEEG |
| AE144_6B | 165 | TSTEPSEGSAPGTSESATPESGPGTSESATPESGPGTSESATPESGPGSEPA |
| | | TSGSETPGSEPATSGSETPGSPAGSPTSTEEGTSTEPSEGSAPGTSTEPSEG |
| | | SAPGSEPATSGSETPGTSESATPESGPGTSTEPSEGSAPG |
| AE288_1 | 166 | GTSESATPESGPGSEPATSGSETPGTSESATPESGPGSEPATSGSETPGTSE |
| | | SATPESGPGTSTEPSEGSAPGSPAGSPTSTEEGTSESATPESGPGSEPATSG |
| | | SETPGTSESATPESGPGSPAGSPTSTEEGSPAGSPTSTEEGTSTEPSEGSAP |
| | | GTSESATPESGPGTSESATPESGPGTSESATPESGPGSEPATSGSETPGSEP |
| | | ATSGSETPGSPAGSPTSTEEGTSTEPSEGSAPGTSTEPSEGSAPGSEPATSG |
| | | SETPGTSESATPESGPGTSTEPSEGSAP |
| AE288_2 | 167 | GSPAGSPTSTEEGTSESATPESGPGSEPATSGSETPGTSESATPESGPGTST |
| | | EPSEGSAPGTSTEPSEGSAPGTSTEPSEGSAPGTSTEPSEGSAPGTSTEPSE |
| | | GSAPGTSTEPSEGSAPGSPAGSPTSTEEGTSTEPSEGSAPGTSESATPESGP |
| | | GSEPATSGSETPGTSESATPESGPGSEPATSGSETPGTSESATPESGPGTST |
| | | EPSEGSAPGTSESATPESGPGSPAGSPTSTEEGSPAGSPTSTEEGSPAGSPT |
| | | STEEGTSESATPESGPGTSTEPSEGSAP |
| AE576 | 168 | GSPAGSPTSTEEGTSESATPESGPGTSTEPSEGSAPGSPAGSPTSTEEGTST |
| | | EPSEGSAPGTSTEPSEGSAPGTSESATPESGPGSEPATSGSETPGSEPATSG |
| | | SETPGSPAGSPTSTEEGTSESATPESGPGTSTEPSEGSAPGTSTEPSEGSAP |
| | | GSPAGSPTSTEEGTSTEPSEGSAPGTSTEPSEGSAPGTSESATPESGPGTST |
| | | EPSEGSAPGTSESATPESGPGSEPATSGSETPGTSTEPSEGSAPGTSTEPSE |
| | | GSAPGTSESATPESGPGTSESATPESGPGSPAGSPTSTEEGTSESATPESGP |
| | | GSEPATSGSETPGTSESATPESGPGTSTEPSEGSAPGTSTEPSEGSAPGTST |
| | | EPSEGSAPGTSTEPSEGSAPGTSTEPSEGSAPGTSTEPSEGSAPGSPAGSPT |
| | | STEEGTSTEPSEGSAPGTSESATPESGPGSEPATSGSETPGTSESATPESGP |
| | | GSEPATSGSETPGTSESATPESGPGTSTEPSEGSAPGTSESATPESGPGSPA |
| | | GSPTSTEEGSPAGSPTSTEEGSPAGSPTSTEEGTSESATPESGPGTSTEPSE |
| | | GSAP |
| AE624 | 169 | MAEPAGSPTSTEEGTPGSGTASSSPGSSTPSGATGSPGASPGTSSTGSPGS |
| | | PAGSPTSTEEGTSESATPESGPGTSTEPSEGSAPGSPAGSPTSTEEGTSTEP |
| | | SEGSAPGTSTEPSEGSAPGTSESATPESGPGSEPATSGSETPGSEPATSGSE |
| | | TPGSPAGSPTSTEEGTSESATPESGPGTSTEPSEGSAPGTSTEPSEGSAPGS |
| | | PAGSPTSTEEGTSTEPSEGSAPGTSTEPSEGSAPGTSESATPESGPGTSTEP |
| | | SEGSAPGTSESATPESGPGSEPATSGSETPGTSTEPSEGSAPGTSTEPSEGS |
| | | APGTSESATPESGPGTSESATPESGPGSPAGSPTSTEEGTSESATPESGPGS |
| | | EPATSGSETPGTSESATPESGPGTSTEPSEGSAPGTSTEPSEGSAPGTSTEP |
| | | SEGSAPGTSTEPSEGSAPGTSTEPSEGSAPGTSTEPSEGSAPGSPAGSPTST |
| | | EEGTSTEPSEGSAPGTSESATPESGPGSEPATSGSETPGTSESATPESGPGS |
| | | EPATSGSETPGTSESATPESGPGTSTEPSEGSAPGTSESATPESGPGSPAGS |
| | | PTSTEEGSPAGSPTSTEEGSPAGSPTSTEEGTSESATPESGPGTSTEPSEGS |
| | | AP |
| AE864 | 170 | GSPAGSPTSTEEGTSESATPESGPGTSTEPSEGSAPGSPAGSPTSTEEGTST |
| | | EPSEGSAPGTSTEPSEGSAPGTSESATPESGPGSEPATSGSETPGSEPATSG |
| | | SETPGSPAGSPTSTEEGTSESATPESGPGTSTEPSEGSAPGTSTEPSEGSAP |
| | | GSPAGSPTSTEEGTSTEPSEGSAPGTSTEPSEGSAPGTSESATPESGPGTST |
| | | EPSEGSAPGTSESATPESGPGSEPATSGSETPGTSTEPSEGSAPGTSTEPSE |
| | | GSAPGTSESATPESGPGTSESATPESGPGSPAGSPTSTEEGTSESATPESGP |
| | | GSEPATSGSETPGTSESATPESGPGTSTEPSEGSAPGTSTEPSEGSAPGTST |
| | | EPSEGSAPGTSTEPSEGSAPGTSTEPSEGSAPGTSTEPSEGSAPGSPAGSPT |
| | | STEEGTSTEPSEGSAPGTSESATPESGPGSEPATSGSETPGTSESATPESGP |
| | | GSEPATSGSETPGTSESATPESGPGTSTEPSEGSAPGTSESATPESGPGSPA |
| | | GSPTSTEEGSPAGSPTSTEEGSPAGSPTSTEEGTSESATPESGPGTSTEPSE |
| | | GSAPGTSESATPESGPGSEPATSGSETPGTSESATPESGPGSEPATSGSETP |
| | | GTSESATPESGPGTSTEPSEGSAPGSPAGSPTSTEEGTSESATPESGPGSEP |
| | | ATSGSETPGTSESATPESGPGSPAGSPTSTEEGSPAGSPTSTEEGTSTEPSE |
| | | GSAPGTSESATPESGPGTSESATPESGPGTSESATPESGPGSEPATSGSETP |
| | | GSEPATSGSETPGSPAGSPTSTEEGTSTEPSEGSAPGTSTEPSEGSAPGSEP |
| | | ATSGSETPGTSESATPESGPGTSTEPSEGSAP |
| AE865 | 171 | GGSPAGSPTSTEEGTSESATPESGPGTSTEPSEGSAPGSPAGSPTSTEEGTS |
| | | TEPSEGSAPGTSTEPSEGSAPGTSESATPESGPGSEPATSGSETPGSEPATS |
| | | GSETPGSPAGSPTSTEEGTSESATPESGPGTSTEPSEGSAPGTSTEPSEGSA |
| | | PGSPAGSPTSTEEGTSTEPSEGSAPGTSTEPSEGSAPGTSESATPESGPGTS |
| | | TEPSEGSAPGTSESATPESGPGSEPATSGSETPGTSTEPSEGSAPGTSTEPS |
| | | EGSAPGTSESATPESGPGTSESATPESGPGSPAGSPTSTEEGTSESATPESG |
| | | PGSEPATSGSETPGTSESATPESGPGTSTEPSEGSAPGTSTEPSEGSAPGTS |
| | | TEPSEGSAPGTSTEPSEGSAPGTSTEPSEGSAPGTSTEPSEGSAPGSPAGSP |
| | | TSTEEGTSTEPSEGSAPGTSESATPESGPGSEPATSGSETPGTSESATPESG |
| | | PGSEPATSGSETPGTSESATPESGPGTSTEPSEGSAPGTSESATPESGPGSP |
| | | AGSPTSTEEGSPAGSPTSTEEGSPAGSPTSTEEGTSESATPESGPGTSTEPS |
| | | EGSAPGTSESATPESGPGSEPATSGSETPGTSESATPESGPGSEPATSGSET |
| | | PGTSESATPESGPGTSTEPSEGSAPGSPAGSPTSTEEGTSESATPESGPGSE |
| | | PATSGSETPGTSESATPESGPGSPAGSPTSTEEGSPAGSPTSTEEGTSTEPS |
| | | EGSAPGTSESATPESGPGTSESATPESGPGTSESATPESGPGSEPATSGSET |
| | | PGSEPATSGSETPGSPAGSPTSTEEGTSTEPSEGSAPGTSTEPSEGSAPGSE |
| | | PATSGSETPGTSESATPESGPGTSTEPSEGSAP |
| AE866 | 172 | PGSPAGSPTSTEEGTSESATPESGPGTSTEPSEGSAPGSPAGSPTSTEEGTS |
| | | TEPSEGSAPGTSTEPSEGSAPGTSESATPESGPGSEPATSGSETPGSEPATS |
| | | GSETPGSPAGSPTSTEEGTSESATPESGPGTSTEPSEGSAPGTSTEPSEGSA |
| | | PGSPAGSPTSTEEGTSTEPSEGSAPGTSTEPSEGSAPGTSESATPESGPGTS |
| | | TEPSEGSAPGTSESATPESGPGSEPATSGSETPGTSTEPSEGSAPGTSTEPS |
| | | EGSAPGTSESATPESGPGTSESATPESGPGSPAGSPTSTEEGTSESATPESG |
| | | PGSEPATSGSETPGTSESATPESGPGTSTEPSEGSAPGTSTEPSEGSAPGTS |
| | | TEPSEGSAPGTSTEPSEGSAPGTSTEPSEGSAPGTSTEPSEGSAPGSPAGSP |
| | | TSTEEGTSTEPSEGSAPGTSESATPESGPGSEPATSGSETPGTSESATPESG |
| | | PGSEPATSGSETPGTSESATPESGPGTSTEPSEGSAPGTSESATPESGPGSP |
| | | AGSPTSTEEGSPAGSPTSTEEGSPAGSPTSTEEGTSESATPESGPGTSTEPS |
| | | EGSAPGTSESATPESGPGSEPATSGSETPGTSESATPESGPGSEPATSGSET |
| | | PGTSESATPESGPGTSTEPSEGSAPGSPAGSPTSTEEGTSESATPESGPGSE |
| | | PATSGSETPGTSESATPESGPGSPAGSPTSTEEGSPAGSPTSTEEGTSTEPS |
| | | EGSAPGTSESATPESGPGTSESATPESGPGTSESATPESGPGSEPATSGSET |
| | | PGSEPATSGSETPGSPAGSPTSTEEGTSTEPSEGSAPGTSTEPSEGSAPGSE |
| | | PATSGSETPGTSESATPESGPGTSTEPSEGSAPG |
| AE1152 | 173 | GSPAGSPTSTEEGTSESATPESGPGTSTEPSEGSAPGSPAGSPTSTEEGTST |
| | | EPSEGSAPGTSTEPSEGSAPGTSESATPESGPGSEPATSGSETPGSEPATSG |
| | | SETPGSPAGSPTSTEEGTSESATPESGPGTSTEPSEGSAPGTSTEPSEGSAP |
| | | GSPAGSPTSTEEGTSTEPSEGSAPGTSTEPSEGSAPGTSESATPESGPGTST |
| | | EPSEGSAPGTSESATPESGPGSEPATSGSETPGTSTEPSEGSAPGTSTEPSE |
| | | GSAPGTSESATPESGPGTSESATPESGPGSPAGSPTSTEEGTSESATPESGP |
| | | GSEPATSGSETPGTSESATPESGPGTSTEPSEGSAPGTSTEPSEGSAPGTST |
| | | EPSEGSAPGTSTEPSEGSAPGTSTEPSEGSAPGTSTEPSEGSAPGSPAGSPT |
| | | STEEGTSTEPSEGSAPGTSESATPESGPGSEPATSGSETPGTSESATPESGP |
| | | GSEPATSGSETPGTSESATPESGPGTSTEPSEGSAPGTSESATPESGPGSPA |
| | | GSPTSTEEGSPAGSPTSTEEGSPAGSPTSTEEGTSESATPESGPGTSTEPSE |
| | | GSAPGTSESATPESGPGSEPATSGSETPGTSESATPESGPGSEPATSGSETP |
| | | GTSESATPESGPGTSTEPSEGSAPGSPAGSPTSTEEGTSESATPESGPGSEP |
| | | ATSGSETPGTSESATPESGPGSPAGSPTSTEEGSPAGSPTSTEEGTSTEPSE |
| | | GSAPGTSESATPESGPGTSESATPESGPGTSESATPESGPGSEPATSGSETP |
| | | GSEPATSGSETPGSPAGSPTSTEEGTSTEPSEGSAPGTSTEPSEGSAPGSEP |
| | | ATSGSETPGTSESATPESGPGTSTEPSEGSAPGTSESATPESGPGSEPATSG |
| | | SETPGTSESATPESGPGSEPATSGSETPGTSESATPESGPGTSTEPSEGSAP |
| | | GSPAGSPTSTEEGTSESATPESGPGSEPATSGSETPGTSESATPESGPGSPA |
| | | GSPTSTEEGSPAGSPTSTEEGTSTEPSEGSAPGTSESATPESGPGTSESATP |
| | | ESGPGTSESATPESGPGSEPATSGSETPGSEPATSGSETPGSPAGSPTSTEE |
| | | GTSTEPSEGSAPGTSTEPSEGSAPGSEPATSGSETPGTSESATPESGPGTST |
| | | EPSEGSAP |
| AE144 | 174 | STEPSEGSAPGSPAGSPTSTEEGTSTEPSEGSAPGTSESATPESGPGSEPAT |
| A | | SGSETPGTSESATPESGPGSEPATSGSETPGTSESATPESGPGTSTEPSEGS |
| | | APGTSESATPESGPGSPAGSPTSTEEGSPAGSPTSTEEGS |
| AE144 | 175 | SEPATSGSETPGTSESATPESGPGSEPATSGSETPGTSESATPESGPGTSTE |
| B | | PSEGSAPGSPAGSPTSTEEGTSESATPESGPGSEPATSGSETPGTSESATPE |
| | | SGPGSPAGSPTSTEEGSPAGSPTSTEEGTSTEPSEGSAPG |
| AE180 | 176 | TSTEEGTSESATPESGPGSEPATSGSETPGTSESATPESGPGSPAGSPTSTE |
| A | | EGSPAGSPTSTEEGTSTEPSEGSAPGTSESATPESGPGTSESATPESGPGTS |
| | | ESATPESGPGSEPATSGSETPGSEPATSGSETPGSPAGSPTSTEEGTSTEPS |
| | | EGSAPGTSTEPSEGSAPGSEPATS |
| AE216 | 177 | PESGPGTSTEPSEGSAPGSPAGSPTSTEEGTSESATPESGPGSEPATSGSET |
| A | | PGTSESATPESGPGSPAGSPTSTEEGSPAGSPTSTEEGTSTEPSEGSAPGTS |
| | | ESATPESGPGTSESATPESGPGTSESATPESGPGSEPATSGSETPGSEPATS |
| | | GSETPGSPAGSPTSTEEGTSTEPSEGSAPGTSTEPSEGSAPGSEPATSGSET |
| | | PGTSESAT |
| AE252 | 178 | ESGPGSEPATSGSETPGTSESATPESGPGTSTEPSEGSAPGSPAGSPTSTEE |
| A | | GTSESATPESGPGSEPATSGSETPGTSESATPESGPGSPAGSPTSTEEGSPA |
| | | GSPTSTEEGTSTEPSEGSAPGTSESATPESGPGTSESATPESGPGTSESATP |
| | | ESGPGSEPATSGSETPGSEPATSGSETPGSPAGSPTSTEEGTSTEPSEGSAP |
| | | GTSTEPSEGSAPGSEPATSGSETPGTSESATPESGPGTSTEPSE |
| AE288 | 179 | TPESGPGTSTEPSEGSAPGTSESATPESGPGSEPATSGSETPGTSESATPES |
| A | | GPGSEPATSGSETPGTSESATPESGPGTSTEPSEGSAPGSPAGSPTSTEEGT |
| | | SESATPESGPGSEPATSGSETPGTSESATPESGPGSPAGSPTSTEEGSPAGS |
| | | PTSTEEGTSTEPSEGSAPGTSESATPESGPGTSESATPESGPGTSESATPES |
| | | GPGSEPATSGSETPGSEPATSGSETPGSPAGSPTSTEEGTSTEPSEGSAPGT |
| | | STEPSEGSAPGSEPATSGSETPGTSESA |
| AE324 | 180 | PESGPGSPAGSPTSTEEGSPAGSPTSTEEGSPAGSPTSTEEGTSESATPESG |
| A | | PGTSTEPSEGSAPGTSESATPESGPGSEPATSGSETPGTSESATPESGPGSE |
| | | PATSGSETPGTSESATPESGPGTSTEPSEGSAPGSPAGSPTSTEEGTSESAT |
| | | PESGPGSEPATSGSETPGTSESATPESGPGSPAGSPTSTEEGSPAGSPTSTE |
| | | EGTSTEPSEGSAPGTSESATPESGPGTSESATPESGPGTSESATPESGPGSE |
| | | PATSGSETPGSEPATSGSETPGSPAGSPTSTEEGTSTEPSEGSAPGTSTEPS |
| | | EGSAPGSEPATS |
| AE360 | 181 | PESGPGTSTEPSEGSAPGTSESATPESGPGSPAGSPTSTEEGSPAGSPTSTE |
| A | | EGSPAGSPTSTEEGTSESATPESGPGTSTEPSEGSAPGTSESATPESGPGSE |
| | | PATSGSETPGTSESATPESGPGSEPATSGSETPGTSESATPESGPGTSTEPS |
| | | EGSAPGSPAGSPTSTEEGTSESATPESGPGSEPATSGSETPGTSESATPESG |
| | | PGSPAGSPTSTEEGSPAGSPTSTEEGTSTEPSEGSAPGTSESATPESGPGTS |
| | | ESATPESGPGTSESATPESGPGSEPATSGSETPGSEPATSGSETPGSPAGSP |
| | | TSTEEGTSTEPSEGSAPGTSTEPSEGSAPGSEPATSGSETPGTSESAT |
| AE396 | 182 | PESGPGSEPATSGSETPGTSESATPESGPGTSTEPSEGSAPGTSESATPESG |
| A | | PGSPAGSPTSTEEGSPAGSPTSTEEGSPAGSPTSTEEGTSESATPESGPGTS |
| | | TEPSEGSAPGTSESATPESGPGSEPATSGSETPGTSESATPESGPGSEPATS |
| | | GSETPGTSESATPESGPGTSTEPSEGSAPGSPAGSPTSTEEGTSESATPESG |
| | | PGSEPATSGSETPGTSESATPESGPGSPAGSPTSTEEGSPAGSPTSTEEGTS |
| | | TEPSEGSAPGTSESATPESGPGTSESATPESGPGTSESATPESGPGSEPATS |
| | | GSETPGSEPATSGSETPGSPAGSPTSTEEGTSTEPSEGSAPGTSTEPS |
| AE432 | 183 | EGSAPGSPAGSPTSTEEGTSTEPSEGSAPGTSESATPESGPGSEPATSGSET |
| A | | PGTSESATPESGPGSEPATSGSETPGTSESATPESGPGTSTEPSEGSAPGTS |
| | | ESATPESGPGSPAGSPTSTEEGSPAGSPTSTEEGSPAGSPTSTEEGTSESAT |
| | | PESGPGTSTEPSEGSAPGTSESATPESGPGSEPATSGSETPGTSESATPESG |
| | | PGSEPATSGSETPGTSESATPESGPGTSTEPSEGSAPGSPAGSPTSTEEGTS |
| | | ESATPESGPGSEPATSGSETPGTSESATPESGPGSPAGSPTSTEEGSPAGSP |
| | | TSTEEGTSTEPSEGSAPGTSESATPESGPGTSESATPESGPGTSESATPESG |
| | | PGSEPATSGSETPGSEPATSGSETPGSPAGSPTSTEEGTSTEPSEGSAPGTS |
| | | TEPSEGSAPGSEPATS |
| AE468 | 184 | EGSAPGTSTEPSEGSAPGTSTEPSEGSAPGSPAGSPTSTEEGTSTEPSEGSA |
| A | | PGTSESATPESGPGSEPATSGSETPGTSESATPESGPGSEPATSGSETPGTS |
| | | ESATPESGPGTSTEPSEGSAPGTSESATPESGPGSPAGSPTSTEEGSPAGSP |
| | | TSTEEGSPAGSPTSTEEGTSESATPESGPGTSTEPSEGSAPGTSESATPESG |
| | | PGSEPATSGSETPGTSESATPESGPGSEPATSGSETPGTSESATPESGPGTS |
| | | TEPSEGSAPGSPAGSPTSTEEGTSESATPESGPGSEPATSGSETPGTSESAT |
| | | PESGPGSPAGSPTSTEEGSPAGSPTSTEEGTSTEPSEGSAPGTSESATPESG |
| | | PGTSESATPESGPGTSESATPESGPGSEPATSGSETPGSEPATSGSETPGSP |
| | | AGSPTSTEEGTSTEPSEGSAPGTSTEPSEGSAPGSEPATSGSETPGTSESAT |
| AE504 | 185 | EGSAPGTSTEPSEGSAPGTSTEPSEGSAPGTSTEPSEGSAPGTSTEPSEGSA |
| A | | PGSPAGSPTSTEEGTSTEPSEGSAPGTSESATPESGPGSEPATSGSETPGTS |
| | | ESATPESGPGSEPATSGSETPGTSESATPESGPGTSTEPSEGSAPGTSESAT |
| | | PESGPGSPAGSPTSTEEGSPAGSPTSTEEGSPAGSPTSTEEGTSESATPESG |
| | | PGTSTEPSEGSAPGTSESATPESGPGSEPATSGSETPGTSESATPESGPGSE |
| | | PATSGSETPGTSESATPESGPGTSTEPSEGSAPGSPAGSPTSTEEGTSESAT |
| | | PESGPGSEPATSGSETPGTSESATPESGPGSPAGSPTSTEEGSPAGSPTSTE |
| | | EGTSTEPSEGSAPGTSESATPESGPGTSESATPESGPGTSESATPESGPGSE |
| | | PATSGSETPGSEPATSGSETPGSPAGSPTSTEEGTSTEPSEGSAPGTSTEPS |
| | | EGSAPGSEPATSGSETPGTSESATPESGPGTSTEPS |
| AE540 | 186 | TPESGPGSPAGSPTSTEEGTSESATPESGPGSEPATSGSETPGTSESATPES |
| A | | GPGTSTEPSEGSAPGTSTEPSEGSAPGTSTEPSEGSAPGTSTEPSEGSAPG |
| | | TSTEPSEGSAPGTSTEPSEGSAPGSPAGSPTSTEEGTSTEPSEGSAPGTSES |
| | | ATPESGPGSEPATSGSETPGTSESATPESGPGSEPATSGSETPGTSESATPE |
| | | SGPGTSTEPSEGSAPGTSESATPESGPGSPAGSPTSTEEGSPAGSPTSTEEG |
| | | SPAGSPTSTEEGTSESATPESGPGTSTEPSEGSAPGTSESATPESGPGSEPA |
| | | TSGSETPGTSESATPESGPGSEPATSGSETPGTSESATPESGPGTSTEPSEG |
| | | SAPGSPAGSPTSTEEGTSESATPESGPGSEPATSGSETPGTSESATPESGPG |
| | | SPAGSPTSTEEGSPAGSPTSTEEGTSTEPSEGSAPGTSESATPESGPGTSES |
| | | ATPESGPGTSESATPESGPGSEPATSGSETPGSEPATSGSETPGSPAGSPTS |
| | | TEEGTSTEPSEGSAPGTSTEP |
| AE576 | 187 | TPESGPGTSESATPESGPGSPAGSPTSTEEGTSESATPESGPGSEPATSGSE |
| A | | TPGTSESATPESGPGTSTEPSEGSAPGTSTEPSEGSAPGTSTEPSEGSAPGT |
| | | STEPSEGSAPGTSTEPSEGSAPGTSTEPSEGSAPGSPAGSPTSTEEGTSTEP |
| | | SEGSAPGTSESATPESGPGSEPATSGSETPGTSESATPESGPGSEPATSGSE |
| | | TPGTSESATPESGPGTSTEPSEGSAPGTSESATPESGPGSPAGSPTSTEEGS |
| | | PAGSPTSTEEGSPAGSPTSTEEGTSESATPESGPGTSTEPSEGSAPGTSESA |
| | | TPESGPGSEPATSGSETPGTSESATPESGPGSEPATSGSETPGTSESATPES |
| | | GPGTSTEPSEGSAPGSPAGSPTSTEEGTSESATPESGPGSEPATSGSETPG |
| | | TSESATPESGPGSPAGSPTSTEEGSPAGSPTSTEEGTSTEPSEGSAPGTSES |
| | | ATPESGPGTSESATPESGPGTSESATPESGPGSEPATSGSETPGSEPATSGS |
| | | ETPGSPAGSPTSTEEGTSTEPSEGSAPGTSTEPSEGSAPGSEPATSGSETPG |
| | | TSESA |
| AE612 | 188 | GSETPGTSTEPSEGSAPGTSTEPSEGSAPGTSESATPESGPGTSESATPESG |
| A | | PGSPAGSPTSTEEGTSESATPESGPGSEPATSGSETPGTSESATPESGPGTS |
| | | TEPSEGSAPGTSTEPSEGSAPGTSTEPSEGSAPGTSTEPSEGSAPGTSTEPS |
| | | EGSAPGTSTEPSEGSAPGSPAGSPTSTEEGTSTEPSEGSAPGTSESATPESG |
| | | PGSEPATSGSETPGTSESATPESGPGSEPATSGSETPGTSESATPESGPGTS |
| | | TEPSEGSAPGTSESATPESGPGSPAGSPTSTEEGSPAGSPTSTEEGSPAGSP |
| | | TSTEEGTSESATPESGPGTSTEPSEGSAPGTSESATPESGPGSEPATSGSET |
| | | PGTSESATPESGPGSEPATSGSETPGTSESATPESGPGTSTEPSEGSAPGSP |
| | | AGSPTSTEEGTSESATPESGPGSEPATSGSETPGTSESATPESGPGSPAGSP |
| | | TSTEEGSPAGSPTSTEEGTSTEPSEGSAPGTSESATPESGPGTSESATPESG |
| | | PGTSESATPESGPGSEPATSGSETPGSEPATSGSETPGSPAGSPTSTEEGTS |
| | | TEPSEGSAPGTSTEPSEGSAPGSEPATSGSETPGTSESAT |
| AE648 | 189 | PESGPGTSTEPSEGSAPGTSESATPESGPGSEPATSGSETPGTSTEPSEGSA |
| A | | PGTSTEPSEGSAPGTSESATPESGPGTSESATPESGPGSPAGSPTSTEEGTS |
| | | ESATPESGPGSEPATSGSETPGTSESATPESGPGTSTEPSEGSAPGTSTEPS |
| | | EGSAPGTSTEPSEGSAPGTSTEPSEGSAPGTSTEPSEGSAPGTSTEPSEGSA |
| | | PGSPAGSPTSTEEGTSTEPSEGSAPGTSESATPESGPGSEPATSGSETPGTS |
| | | ESATPESGPGSEPATSGSETPGTSESATPESGPGTSTEPSEGSAPGTSESAT |
| | | PESGPGSPAGSPTSTEEGSPAGSPTSTEEGSPAGSPTSTEEGTSESATPESG |
| | | PGTSTEPSEGSAPGTSESATPESGPGSEPATSGSETPGTSESATPESGPGSE |
| | | PATSGSETPGTSESATPESGPGTSTEPSEGSAPGSPAGSPTSTEEGTSESAT |
| | | PESGPGSEPATSGSETPGTSESATPESGPGSPAGSPTSTEEGSPAGSPTSTE |
| | | EGTSTEPSEGSAPGTSESATPESGPGTSESATPESGPGTSESATPESGPGSE |
| | | PATSGSETPGSEPATSGSETPGSPAGSPTSTEEGTSTEPSEGSAPGTSTEPS |
| | | EGSAPGSEPATSGSETPGTSESAT |
| AE684 | 190 | EGSAPGSPAGSPTSTEEGTSTEPSEGSAPGTSTEPSEGSAPGTSESATPESG |
| A | | PGTSTEPSEGSAPGTSESATPESGPGSEPATSGSETPGTSTEPSEGSAPGTS |
| | | TEPSEGSAPGTSESATPESGPGTSESATPESGPGSPAGSPTSTEEGTSESAT |
| | | PESGPGSEPATSGSETPGTSESATPESGPGTSTEPSEGSAPGTSTEPSEGSA |
| | | PGTSTEPSEGSAPGTSTEPSEGSAPGTSTEPSEGSAPGTSTEPSEGSAPGSP |
| | | AGSPTSTEEGTSTEPSEGSAPGTSESATPESGPGSEPATSGSETPGTSESAT |
| | | PESGPGSEPATSGSETPGTSESATPESGPGTSTEPSEGSAPGTSESATPESG |
| | | PGSPAGSPTSTEEGSPAGSPTSTEEGSPAGSPTSTEEGTSESATPESGPGTS |
| | | TEPSEGSAPGTSESATPESGPGSEPATSGSETPGTSESATPESGPGSEPATS |
| | | GSETPGTSESATPESGPGTSTEPSEGSAPGSPAGSPTSTEEGTSESATPESG |
| | | PGSEPATSGSETPGTSESATPESGPGSPAGSPTSTEEGSPAGSPTSTEEGTS |
| | | TEPSEGSAPGTSESATPESGPGTSESATPESGPGTSESATPESGPGSEPATS |
| | | GSETPGSEPATSGSETPGSPAGSPTSTEEGTSTEPSEGSAPGTSTEPSEGSA |
| | | PGSEPATS |
| AE720 | 191 | TSGSETPGSEPATSGSETPGSPAGSPTSTEEGTSESATPESGPGTSTEPSEG |
| A | | SAPGTSTEPSEGSAPGSPAGSPTSTEEGTSTEPSEGSAPGTSTEPSEGSAPG |
| | | TSESATPESGPGTSTEPSEGSAPGTSESATPESGPGSEPATSGSETPGTSTE |
| | | PSEGSAPGTSTEPSEGSAPGTSESATPESGPGTSESATPESGPGSPAGSPTS |
| | | TEEGTSESATPESGPGSEPATSGSETPGTSESATPESGPGTSTEPSEGSAPG |
| | | TSTEPSEGSAPGTSTEPSEGSAPGTSTEPSEGSAPGTSTEPSEGSAPGTSTE |
| | | PSEGSAPGSPAGSPTSTEEGTSTEPSEGSAPGTSESATPESGPGSEPATSGS |
| | | ETPGTSESATPESGPGSEPATSGSETPGTSESATPESGPGTSTEPSEGSAPG |
| | | TSESATPESGPGSPAGSPTSTEEGSPAGSPTSTEEGSPAGSPTSTEEGTSES |
| | | ATPESGPGTSTEPSEGSAPGTSESATPESGPGSEPATSGSETPGTSESATPE |
| | | SGPGSEPATSGSETPGTSESATPESGPGTSTEPSEGSAPGSPAGSPTSTEEG |
| | | TSESATPESGPGSEPATSGSETPGTSESATPESGPGSPAGSPTSTEEGSPAG |
| | | SPTSTEEGTSTEPSEGSAPGTSESATPESGPGTSESATPESGPGTSESATPE |
| | | SGPGSEPATSGSETPGSEPATSGSETPGSPAGSPTSTEEGTSTE |
| AE756 | 192 | TSGSETPGSEPATSGSETPGSPAGSPTSTEEGTSESATPESGPGTSTEPSEG |
| A | | SAPGTSTEPSEGSAPGSPAGSPTSTEEGTSTEPSEGSAPGTSTEPSEGSAPG |
| | | TSESATPESGPGTSTEPSEGSAPGTSESATPESGPGSEPATSGSETPGTSTE |
| | | PSEGSAPGTSTEPSEGSAPGTSESATPESGPGTSESATPESGPGSPAGSPTS |
| | | TEEGTSESATPESGPGSEPATSGSETPGTSESATPESGPGTSTEPSEGSAPG |
| | | TSTEPSEGSAPGTSTEPSEGSAPGTSTEPSEGSAPGTSTEPSEGSAPGTSTE |
| | | PSEGSAPGSPAGSPTSTEEGTSTEPSEGSAPGTSESATPESGPGSEPATSGS |
| | | ETPGTSESATPESGPGSEPATSGSETPGTSESATPESGPGTSTEPSEGSAPG |
| | | TSESATPESGPGSPAGSPTSTEEGSPAGSPTSTEEGSPAGSPTSTEEGTSES |
| | | ATPESGPGTSTEPSEGSAPGTSESATPESGPGSEPATSGSETPGTSESATPE |
| | | SGPGSEPATSGSETPGTSESATPESGPGTSTEPSEGSAPGSPAGSPTSTEEG |
| | | TSESATPESGPGSEPATSGSETPGTSESATPESGPGSPAGSPTSTEEGSPAG |
| | | SPTSTEEGTSTEPSEGSAPGTSESATPESGPGTSESATPESGPGTSESATPE |
| | | SGPGSEPATSGSETPGSEPATSGSETPGSPAGSPTSTEEGTSTEPSEGSAPG |
| | | TSTEPSEGSAPGSEPATSGSETPGTSES |
| AE792 | 193 | EGSAPGTSESATPESGPGSEPATSGSETPGSEPATSGSETPGSPAGSPTSTE |
| A | | EGTSESATPESGPGTSTEPSEGSAPGTSTEPSEGSAPGSPAGSPTSTEEGTS |
| | | TEPSEGSAPGTSTEPSEGSAPGTSESATPESGPGTSTEPSEGSAPGTSESAT |
| | | PESGPGSEPATSGSETPGTSTEPSEGSAPGTSTEPSEGSAPGTSESATPESG |
| | | PGTSESATPESGPGSPAGSPTSTEEGTSESATPESGPGSEPATSGSETPGTS |
| | | ESATPESGPGTSTEPSEGSAPGTSTEPSEGSAPGTSTEPSEGSAPGTSTEPS |
| | | EGSAPGTSTEPSEGSAPGTSTEPSEGSAPGSPAGSPTSTEEGTSTEPSEGSA |
| | | PGTSESATPESGPGSEPATSGSETPGTSESATPESGPGSEPATSGSETPGTS |
| | | ESATPESGPGTSTEPSEGSAPGTSESATPESGPGSPAGSPTSTEEGSPAGSP |
| | | TSTEEGSPAGSPTSTEEGTSESATPESGPGTSTEPSEGSAPGTSESATPESG |
| | | PGSEPATSGSETPGTSESATPESGPGSEPATSGSETPGTSESATPESGPGTS |
| | | TEPSEGSAPGSPAGSPTSTEEGTSESATPESGPGSEPATSGSETPGTSESAT |
| | | PESGPGSPAGSPTSTEEGSPAGSPTSTEEGTSTEPSEGSAPGTSESATPESG |
| | | PGTSESATPESGPGTSESATPESGPGSEPATSGSETPGSEPATSGSETPGSP |
| | | AGSPTSTEEGTSTEPSEGSAPGTSTEPSEGSAPGSEPATSGSETPGTSESAT |
| | | PESGPGTSTEPS |
| AE828 | 194 | PESGPGTSTEPSEGSAPGSPAGSPTSTEEGTSTEPSEGSAPGTSTEPSEGSA |
| A | | PGTSESATPESGPGSEPATSGSETPGSEPATSGSETPGSPAGSPTSTEEGTS |
| | | ESATPESGPGTSTEPSEGSAPGTSTEPSEGSAPGSPAGSPTSTEEGTSTEPS |
| | | EGSAPGTSTEPSEGSAPGTSESATPESGPGTSTEPSEGSAPGTSESATPESG |
| | | PGSEPATSGSETPGTSTEPSEGSAPGTSTEPSEGSAPGTSESATPESGPGTS |
| | | ESATPESGPGSPAGSPTSTEEGTSESATPESGPGSEPATSGSETPGTSESAT |
| | | PESGPGTSTEPSEGSAPGTSTEPSEGSAPGTSTEPSEGSAPGTSTEPSEGSA |
| | | PGTSTEPSEGSAPGTSTEPSEGSAPGSPAGSPTSTEEGTSTEPSEGSAPGTS |
| | | ESATPESGPGSEPATSGSETPGTSESATPESGPGSEPATSGSETPGTSESAT |
| | | PESGPGTSTEPSEGSAPGTSESATPESGPGSPAGSPTSTEEGSPAGSPTSTE |
| | | EGSPAGSPTSTEEGTSESATPESGPGTSTEPSEGSAPGTSESATPESGPGSE |
| | | PATSGSETPGTSESATPESGPGSEPATSGSETPGTSESATPESGPGTSTEPS |
| | | EGSAPGSPAGSPTSTEEGTSESATPESGPGSEPATSGSETPGTSESATPESG |
| | | PGSPAGSPTSTEEGSPAGSPTSTEEGTSTEPSEGSAPGTSESATPESGPGTS |
| | | ESATPESGPGTSESATPESGPGSEPATSGSETPGSEPATSGSETPGSPAGSP |
| | | TSTEEGTSTEPSEGSAPGTSTEPSEGSAPGSEPATSGSETPGTSESAT |
| AE869 | 195 | GSPGSPAGSPTSTEEGTSESATPESGPGTSTEPSEGSAPGSPAGSPTSTEEG |
| | | TSTEPSEGSAPGTSTEPSEGSAPGTSESATPESGPGSEPATSGSETPGSEPA |
| | | TSGSETPGSPAGSPTSTEEGTSESATPESGPGTSTEPSEGSAPGTSTEPSEG |
| | | SAPGSPAGSPTSTEEGTSTEPSEGSAPGTSTEPSEGSAPGTSESATPESGPG |
| | | TSTEPSEGSAPGTSESATPESGPGSEPATSGSETPGTSTEPSEGSAPGTSTE |
| | | PSEGSAPGTSESATPESGPGTSESATPESGPGSPAGSPTSTEEGTSESATPE |
| | | SGPGSEPATSGSETPGTSESATPESGPGTSTEPSEGSAPGTSTEPSEGSAPG |
| | | TSTEPSEGSAPGTSTEPSEGSAPGTSTEPSEGSAPGTSTEPSEGSAPGSPAG |
| | | SPTSTEEGTSTEPSEGSAPGTSESATPESGPGSEPATSGSETPGTSESATPE |
| | | SGPGSEPATSGSETPGTSESATPESGPGTSTEPSEGSAPGTSESATPESGPG |
| | | SPAGSPTSTEEGSPAGSPTSTEEGSPAGSPTSTEEGTSESATPESGPGTSTE |
| | | PSEGSAPGTSESATPESGPGSEPATSGSETPGTSESATPESGPGSEPATSGS |
| | | ETPGTSESATPESGPGTSTEPSEGSAPGSPAGSPTSTEEGTSESATPESGPG |
| | | SEPATSGSETPGTSESATPESGPGSPAGSPTSTEEGSPAGSPTSTEEGTSTE |
| | | PSEGSAPGTSESATPESGPGTSESATPESGPGTSESATPESGPGSEPATSGS |
| | | ETPGSEPATSGSETPGSPAGSPTSTEEGTSTEPSEGSAPGTSTEPSEGSAPG |
| | | SEPATSGSETPGTSESATPESGPGTSTEPSEGSAPGR |
| AE144_R1 | 196 | SAGSPGSPAGSPTSTEEGTSESATPESGPGTSTEPSEGSAPGSPAGSPTSTE |
| | | EGTSTEPSEGSAPGTSTEPSEGSAPGTSESATPESGPGSEPATSGSETPGSE |
| | | PATSGSETPGSPAGSPTSTEEGTSESATPESGPGTESASR |
| AE288_R1 | 197 | SAGSPTGPGSEPATSGSETPGTSESATPESGPGSEPATSGSETPGTSESATP |
| | | ESGPGTSTEPSEGSAPGSPAGSPTSTEEGTSESATPESGPGSEPATSGSETP |
| | | GTSESATPESGPGSPAGSPTSTEEGSPAGSPTSTEEGTSTEPSEGSAPGTSE |
| | | SATPESGPGTSESATPESGPGTSESATPESGPGSEPATSGSETPGSEPATSG |
| | | SETPGSPAGSPTSTEEGTSTEPSEGSAPGTSTEPSEGSAPGSEPATSGSETP |
| | | GTSESATPESGPGTSTEPSEGSAPSASR |
| AE432_R1 | 198 | SAGSPGSPAGSPTSTEEGTSESATPESGPGTSTEPSEGSAPGSPAGSPTSTE |
| | | EGTSTEPSEGSAPGTSTEPSEGSAPGTSESATPESGPGSEPATSGSETPGSE |
| | | PATSGSETPGSPAGSPTSTEEGTSESATPESGPGTSTEPSEGSAPGTSTEPS |
| | | EGSAPGSPAGSPTSTEEGTSTEPSEGSAPGTSTEPSEGSAPGTSESATPESG |
| | | PGTSTEPSEGSAPGTSESATPESGPGSEPATSGSETPGTSTEPSEGSAPGTS |
| | | TEPSEGSAPGTSESATPESGPGTSESATPESGPGSPAGSPTSTEEGTSESAT |
| | | PESGPGSEPATSGSETPGTSESATPESGPGTSTEPSEGSAPGTSTEPSEGSA |
| | | PGTSTEPSEGSAPGTSTEPSEGSAPGTSTEPSEGSAPGTSTEPSEGSAPGSP |
| | | AGSPTSTEEGTESASR |
| AE576_R1 | 199 | SAGSPTEEGTSESATPESGPGSEPATSGSETPGTSESATPESGPGTSTEPSE |
| | | GSAPGTSTEPSEGSAPGTSTEPSEGSAPGTSTEPSEGSAPGTSTEPSEGSAP |
| | | GTSTEPSEGSAPGSPAGSPTSTEEGTSTEPSEGSAPGTSESATPESGPGSEP |
| | | ATSGSETPGTSESATPESGPGSEPATSGSETPGTSESATPESGPGTSTEPSE |
| | | GSAPGTSESATPESGPGSPAGSPTSTEEGSPAGSPTSTEEGSPAGSPTSTEE |
| | | GTSESATPESGPGTSTEPSEGSAPGTSESATPESGPGSEPATSGSETPGTSE |
| | | SATPESGPGSEPATSGSETPGTSESATPESGPGTSTEPSEGSAPGSPAGSPT |
| | | STEEGTSESATPESGPGSEPATSGSETPGTSESATPESGPGSPAGSPTSTEE |
| | | GSPAGSPTSTEEGTSTEPSEGSAPGTSESATPESGPGTSESATPESGPGTSE |
| | | SATPESGPGSEPATSGSETPGSEPATSGSETPGSPAGSPTSTEEGTSTEPSE |
| | | GSAPGTSTEPSEGSAPGSEPATSGSETPGTSESATPESGPGTSTEPSEGSAP |
| | | SASR |
| AE864_R1 | 200 | SAGSPGSPAGSPTSTEEGTSESATPESGPGTSTEPSEGSAPGSPAGSPTSTE |
| | | EGTSTEPSEGSAPGTSTEPSEGSAPGTSESATPESGPGSEPATSGSETPGSE |
| | | PATSGSETPGSPAGSPTSTEEGTSESATPESGPGTSTEPSEGSAPGTSTEPS |
| | | EGSAPGSPAGSPTSTEEGTSTEPSEGSAPGTSTEPSEGSAPGTSESATPESG |
| | | PGTSTEPSEGSAPGTSESATPESGPGSEPATSGSETPGTSTEPSEGSAPGTS |
| | | TEPSEGSAPGTSESATPESGPGTSESATPESGPGSPAGSPTSTEEGTSESAT |
| | | PESGPGSEPATSGSETPGTSESATPESGPGTSTEPSEGSAPGTSTEPSEGSA |
| | | PGTSTEPSEGSAPGTSTEPSEGSAPGTSTEPSEGSAPGTSTEPSEGSAPGSP |
| | | AGSPTSTEEGTSTEPSEGSAPGTSESATPESGPGSEPATSGSETPGTSESAT |
| | | PESGPGSEPATSGSETPGTSESATPESGPGTSTEPSEGSAPGTSESATPESG |
| | | PGSPAGSPTSTEEGSPAGSPTSTEEGSPAGSPTSTEEGTSESATPESGPGTS |
| | | TEPSEGSAPGTSESATPESGPGSEPATSGSETPGTSESATPESGPGSEPATS |
| | | GSETPGTSESATPESGPGTSTEPSEGSAPGSPAGSPTSTEEGTSESATPESG |
| | | PGSEPATSGSETPGTSESATPESGPGSPAGSPTSTEEGSPAGSPTSTEEGTS |
| | | TEPSEGSAPGTSESATPESGPGTSESATPESGPGTSESATPESGPGSEPATS |
| | | GSETPGSEPATSGSETPGSPAGSPTSTEEGTSTEPSEGSAPGTSTEPSEGSA |
| | | PGSEPATSGSETPGTSESATPESGPGTESASR |
| AE712 | 201 | PGSPAGSPTSTEEGTSESATPESGPGTSTEPSEGSAPGSPAGSPTSTEEGTS |
| | | TEPSEGSAPGTSTEPSEGSAPGTSESATPESGPGSEPATSGSETPGSEPATS |
| | | GSETPGSPAGSPTSTEEGTSESATPESGPGTSTEPSEGSAPGTSTEPSEGSA |
| | | PGSPAGSPTSTEEGTSTEPSEGSAPGTSTEPSEGSAPGTSESATPESGPGTS |
| | | TEPSEGSAPGTSESATPESGPGSEPATSGSETPGTSTEPSEGSAPGTSTEPS |
| | | EGSAPGTSESATPESGPGTSESATPESGPGSPAGSPTSTEEGTSESATPESG |
| | | PGSEPATSGSETPGTSESATPESGPGTSTEPSEGSAPGTSTEPSEGSAPGTS |
| | | TEPSEGSAPGTSTEPSEGSAPGTSTEPSEGSAPGTSTEPSEGSAPGSPAGSP |
| | | TSTEEGTSTEPSEGSAPGTSESATPESGPGSEPATSGSETPGTSESATPESG |
| | | PGSEPATSGSETPGTSESATPESGPGTSTEPSEGSAPGTSESATPESGPGSP |
| | | AGSPTSTEEGSPAGSPTSTEEGSPAGSPTSTEEGTSESATPESGPGTSTEPS |
| | | EGSAPGTSESATPESGPGSEPATSGSETPGTSESATPESGPGSEPATSGSET |
| | | PGTSESATPESGPGTSTEPSEGSAPGSPAGSPTSTEEGTSESATPESGPGSE |
| | | PATSGSETPGTSESATPESGPGSPAGSPTSTEAHHH |
| AE864_R2 | 202 | GSPGAGSPAGSPTSTEEGTSESATPESGPGTSTEPSEGSAPGSPAGSPTSTE |
| | | EGTSTEPSEGSAPGTSTEPSEGSAPGTSESATPESGPGSEPATSGSETPGSE |
| | | PATSGSETPGSPAGSPTSTEEGTSESATPESGPGTSTEPSEGSAPGTSTEPS |
| | | EGSAPGSPAGSPTSTEEGTSTEPSEGSAPGTSTEPSEGSAPGTSESATPESG |
| | | PGTSTEPSEGSAPGTSESATPESGPGSEPATSGSETPGTSTEPSEGSAPGTS |
| | | TEPSEGSAPGTSESATPESGPGTSESATPESGPGSPAGSPTSTEEGTSESAT |
| | | PESGPGSEPATSGSETPGTSESATPESGPGTSTEPSEGSAPGTSTEPSEGSA |
| | | PGTSTEPSEGSAPGTSTEPSEGSAPGTSTEPSEGSAPGTSTEPSEGSAPGSP |
| | | AGSPTSTEEGTSTEPSEGSAPGTSESATPESGPGSEPATSGSETPGTSESAT |
| | | PESGPGSEPATSGSETPGTSESATPESGPGTSTEPSEGSAPGTSESATPESG |
| | | PGSPAGSPTSTEEGSPAGSPTSTEEGSPAGSPTSTEEGTSESATPESGPGTS |
| | | TEPSEGSAPGTSESATPESGPGSEPATSGSETPGTSESATPESGPGSEPATS |
| | | GSETPGTSESATPESGPGTSTEPSEGSAPGSPAGSPTSTEEGTSESATPESG |
| | | PGSEPATSGSETPGTSESATPESGPGSPAGSPTSTEEGSPAGSPTSTEEGTS |
| | | TEPSEGSAPGTSESATPESGPGTSESATPESGPGTSESATPESGPGSEPATS |
| | | GSETPGSEPATSGSETPGSPAGSPTSTEEGTSTEPSEGSAPGTSTEPSEGSA |
| | | PGSEPATSGSETPGTSESATPESGPGTESASR |
| AE288_3 | 203 | SPAGSPTSTEEGTSESATPESGPGTSTEPSEGSAPGTSESATPESGPGSEPA |
| | | TSGSETPGTSESATPESGPGSEPATSGSETPGTSESATPESGPGTSTEPSEG |
| | | SAPGSPAGSPTSTEEGTSESATPESGPGSEPATSGSETPGTSESATPESGPG |
| | | SPAGSPTSTEEGSPAGSPTSTEEGTSTEPSEGSAPGTSESATPESGPGTSES |
| | | ATPESGPGTSESATPESGPGSEPATSGSETPGSEPATSGSETPGSPAGSPTS |
| | | TEEGTSTEPSEGSAPGTSTEPSEGSAPG |
| AE284 | 204 | GTSESATPESGPGSEPATSGSETPGTSESATPESGPGSEPATSGSETPGTSE |
| | | SATPESGPGTSTEPSEGSAPGSPAGSPTSTEEGTSESATPESGPGSEPATSG |
| | | SETPGTSESATPESGPGSPAGSPTSTEEGSPAGSPTSTEEGTSTEPSEGSAP |
| | | GTSESATPESGPGTSESATPESGPGTSESATPESGPGSEPATSGSETPGSEP |
| | | ATSGSETPGSPAGSPTSTEEGTSTEPSEGSAPGTSTEPSEGSAPGSEPATSG |
| | | SETPGTSESATPESGPGTSTEPSE |
| AE292 | 205 | SPAGSPTSTEEGTSESATPESGPGTSTEPSEGSAPGTSESATPESGPGSEPA |
| | | TSGSETPGTSESATPESGPGSEPATSGSETPGTSESATPESGPGTSTEPSEG |
| | | SAPGSPAGSPTSTEEGTSESATPESGPGSEPATSGSETPGTSESATPESGPG |
| | | SPAGSPTSTEEGSPAGSPTSTEEGTSTEPSEGSAPGTSESATPESGPGTSES |
| | | ATPESGPGTSESATPESGPGSEPATSGSETPGSEPATSGSETPGSPAGSPTS |
| | | TEEGTSTEPSEGSAPGTSTEPSEGSAPGGSAP |
| AE293 | 206 | PGSPAGSPTSTEEGTSESATPESGPGTSTEPSEGSAPGSPAGSPTSTEEGTS |
| | | TEPSEGSAPGTSTEPSEGSAPGTSESATPESGPGSEPATSGSETPGSEPATS |
| | | GSETPGSPAGSPTSTEEGTSESATPESGPGTSTEPSEGSAPGTSTEPSEGSA |
| | | PGSPAGSPTSTEEGTSTEPSEGSAPGTSTEPSEGSAPGTSESATPESGPGTS |
| | | TEPSEGSAPGTSESATPESGPGSEPATSGSETPGTSTEPSEGSAPGTSTEPS |
| | | EGSAPGTSESATPESGPGTSESATPEGAAEPEA |
| AE300 | 207 | PGSPAGSPTSTEEGTSESATPESGPGTSTEPSEGSAPGSPAGSPTSTEEGTS |
| | | TEPSEGSAPGTSTEPSEGSAPGTSESATPESGPGSEPATSGSETPGSEPATS |
| | | GSETPGSPAGSPTSTEEGTSESATPESGPGTSTEPSEGSAPGTSTEPSEGSA |
| | | PGSPAGSPTSTEEGTSTEPSEGSAPGTSTEPSEGSAPGTSESATPESGPGTS |
| | | TEPSEGSAPGTSESATPESGPGSEPATSGSETPGTSTEPSEGSAPGTSTEPS |
| | | EGSAPGTSESATPESGPGTSESATPESGPGSPAGAAEPEA |
| AE864_2 | 208 | AGSPTSTEEGTSESATPESGPGTSTEPSEGSAPGSPAGSPTSTEEGTSTEPS |
| | | EGSAPGTSTEPSEGSAPGTSESATPESGPGSEPATSGSETPGSEPATSGSET |
| | | PGSPAGSPTSTEEGTSESATPESGPGTSTEPSEGSAPGTSTEPSEGSAPGSP |
| | | AGSPTSTEEGTSTEPSEGSAPGTSTEPSEGSAPGTSESATPESGPGTSTEPS |
| | | EGSAPGTSESATPESGPGSEPATSGSETPGTSTEPSEGSAPGTSTEPSEGSA |
| | | PGTSESATPESGPGTSESATPESGPGSPAGSPTSTEEGTSESATPESGPGSE |
| | | PATSGSETPGTSESATPESGPGTSTEPSEGSAPGTSTEPSEGSAPGTSTEPS |
| | | EGSAPGTSTEPSEGSAPGTSTEPSEGSAPGTSTEPSEGSAPGSPAGSPTSTE |
| | | EGTSTEPSEGSAPGTSESATPESGPGSEPATSGSETPGTSESATPESGPGSE |
| | | PATSGSETPGTSESATPESGPGTSTEPSEGSAPGTSESATPESGPGSPAGSP |
| | | TSTEEGSPAGSPTSTEEGSPAGSPTSTEEGTSESATPESGPGTSTEPSEGSA |
| | | PGTSESATPESGPGSEPATSGSETPGTSESATPESGPGSEPATSGSETPGTS |
| | | ESATPESGPGTSTEPSEGSAPGSPAGSPTSTEEGTSESATPESGPGSEPATS |
| | | GSETPGTSESATPESGPGSPAGSPTSTEEGSPAGSPTSTEEGTSTEPSEGSA |
| | | PGTSESATPESGPGTSESATPESGPGTSESATPESGPGSEPATSGSETPGSE |
| | | PATSGSETPGSPAGSPTSTEEGTSTEPSEGSAPGTSTEPSEGSAPGSEPATS |
| | | GSETPGTSESATPESGPGTSTEPSEGAAEPEA |
| AE867 | 209 | GSPAGSPTSTEEGTSESATPESGPGTSTEPSEGSAPGSPAGSPTSTEEGTST |
| | | EPSEGSAPGTSTEPSEGSAPGTSESATPESGPGSEPATSGSETPGSEPATSG |
| | | SETPGSPAGSPTSTEEGTSESATPESGPGTSTEPSEGSAPGTSTEPSEGSAP |
| | | GSPAGSPTSTEEGTSTEPSEGSAPGTSTEPSEGSAPGTSESATPESGPGTST |
| | | EPSEGSAPGTSESATPESGPGSEPATSGSETPGTSTEPSEGSAPGTSTEPSE |
| | | GSAPGTSESATPESGPGTSESATPESGPGSPAGSPTSTEEGTSESATPESGP |
| | | GSEPATSGSETPGTSESATPESGPGTSTEPSEGSAPGTSTEPSEGSAPGTST |
| | | EPSEGSAPGTSTEPSEGSAPGTSTEPSEGSAPGTSTEPSEGSAPGSPAGSPT |
| | | STEEGTSTEPSEGSAPGTSESATPESGPGSEPATSGSETPGTSESATPESGP |
| | | GSEPATSGSETPGTSESATPESGPGTSTEPSEGSAPGTSESATPESGPGSPA |
| | | GSPTSTEEGSPAGSPTSTEEGSPAGSPTSTEEGTSESATPESGPGTSTEPSE |
| | | GSAPGTSESATPESGPGSEPATSGSETPGTSESATPESGPGSEPATSGSETP |
| | | GTSESATPESGPGTSTEPSEGSAPGSPAGSPTSTEEGTSESATPESGPGSEP |
| | | ATSGSETPGTSESATPESGPGSPAGSPTSTEEGSPAGSPTSTEEGTSTEPSE |
| | | GSAPGTSESATPESGPGTSESATPESGPGTSESATPESGPGSEPATSGSETP |
| | | GSEPATSGSETPGSPAGSPTSTEEGTSTEPSEGSAPGTSTEPSEGSAPGSEP |
| | | ATSGSETPGTSESATPESGPGTSTEPSEGAAEPEA |
| AE867_2 | 210 | SPGSPAGSPTSTEEGTSESATPESGPGTSTEPSEGSAPGSPAGSPTSTEEGT |
| | | STEPSEGSAPGTSTEPSEGSAPGTSESATPESGPGSEPATSGSETPGSEPAT |
| | | SGSETPGSPAGSPTSTEEGTSESATPESGPGTSTEPSEGSAPGTSTEPSEGS |
| | | APGSPAGSPTSTEEGTSTEPSEGSAPGTSTEPSEGSAPGTSESATPESGPGT |
| | | STEPSEGSAPGTSESATPESGPGSEPATSGSETPGTSTEPSEGSAPGTSTEP |
| | | SEGSAPGTSESATPESGPGTSESATPESGPGSPAGSPTSTEEGTSESATPES |
| | | GPGSEPATSGSETPGTSESATPESGPGTSTEPSEGSAPGTSTEPSEGSAPG |
| | | TSTEPSEGSAPGTSTEPSEGSAPGTSTEPSEGSAPGTSTEPSEGSAPGSPAG |
| | | SPTSTEEGTSTEPSEGSAPGTSESATPESGPGSEPATSGSETPGTSESATPE |
| | | SGPGSEPATSGSETPGTSESATPESGPGTSTEPSEGSAPGTSESATPESGPG |
| | | SPAGSPTSTEEGSPAGSPTSTEEGSPAGSPTSTEEGTSESATPESGPGTSTE |
| | | PSEGSAPGTSESATPESGPGSEPATSGSETPGTSESATPESGPGSEPATSGS |
| | | ETPGTSESATPESGPGTSTEPSEGSAPGSPAGSPTSTEEGTSESATPESGPG |
| | | SEPATSGSETPGTSESATPESGPGSPAGSPTSTEEGSPAGSPTSTEEGTSTE |
| | | PSEGSAPGTSESATPESGPGTSESATPESGPGTSESATPESGPGSEPATSGS |
| | | ETPGSEPATSGSETPGSPAGSPTSTEEGTSTEPSEGSAPGTSTEPSEGSAPG |
| | | SEPATSGSETPGTSESATPESGPGTSTEPSEGSAPG |
| AE868 | 211 | PGSPAGSPTSTEEGTSESATPESGPGTSTEPSEGSAPGSPAGSPTSTEEGTS |
| | | TEPSEGSAPGTSTEPSEGSAPGTSESATPESGPGSEPATSGSETPGSEPATS |
| | | GSETPGSPAGSPTSTEEGTSESATPESGPGTSTEPSEGSAPGTSTEPSEGSA |
| | | PGSPAGSPTSTEEGTSTEPSEGSAPGTSTEPSEGSAPGTSESATPESGPGTS |
| | | TEPSEGSAPGTSESATPESGPGSEPATSGSETPGTSTEPSEGSAPGTSTEPS |
| | | EGSAPGTSESATPESGPGTSESATPESGPGSPAGSPTSTEEGTSESATPESG |
| | | PGSEPATSGSETPGTSESATPESGPGTSTEPSEGSAPGTSTEPSEGSAPGTS |
| | | TEPSEGSAPGTSTEPSEGSAPGTSTEPSEGSAPGTSTEPSEGSAPGSPAGSP |
| | | TSTEEGTSTEPSEGSAPGTSESATPESGPGSEPATSGSETPGTSESATPESG |
| | | PGSEPATSGSETPGTSESATPESGPGTSTEPSEGSAPGTSESATPESGPGSP |
| | | AGSPTSTEEGSPAGSPTSTEEGSPAGSPTSTEEGTSESATPESGPGTSTEPS |
| | | EGSAPGTSESATPESGPGSEPATSGSETPGTSESATPESGPGSEPATSGSET |
| | | PGTSESATPESGPGTSTEPSEGSAPGSPAGSPTSTEEGTSESATPESGPGSE |
| | | PATSGSETPGTSESATPESGPGSPAGSPTSTEEGSPAGSPTSTEEGTSTEPS |
| | | EGSAPGTSESATPESGPGTSESATPESGPGTSESATPESGPGSEPATSGSET |
| | | PGSEPATSGSETPGSPAGSPTSTEEGTSTEPSEGSAPGTSTEPSEGSAPGSE |
| | | PATSGSETPGTSESATPESGPGTSTEPSEGAAEPEA |
| AE584 | 212 | PGSPAGSPTSTEEGTSESATPESGPGTSTEPSEGSAPGSPAGSPTSTEEGTS |
| | | TEPSEGSAPGTSTEPSEGSAPGTSESATPESGPGSEPATSGSETPGSEPATS |
| | | GSETPGSPAGSPTSTEEGTSESATPESGPGTSTEPSEGSAPGTSTEPSEGSA |
| | | PGSPAGSPTSTEEGTSTEPSEGSAPGTSTEPSEGSAPGTSESATPESGPGTS |
| | | TEPSEGSAPGTSESATPESGPGSEPATSGSETPGTSTEPSEGSAPGTSTEPS |
| | | EGSAPGTSESATPESGPGTSESATPESGPGSPAGSPTSTEEGTSESATPESG |
| | | PGSEPATSGSETPGTSESATPESGPGTSTEPSEGSAPGTSTEPSEGSAPGTS |
| | | TEPSEGSAPGTSTEPSEGSAPGTSTEPSEGSAPGTSTEPSEGSAPGSPAGSP |
| | | TSTEEGTSTEPSEGSAPGTSESATPESGPGSEPATSGSETPGTSESATPESG |
| | | PGSEPATSGSETPGTSESATPESGPGTSTEPSEGSAPGTSESATPESGPGSP |
| | | AGSPTSTEEGSPAGSPTSTEEGSPAGSPTSTEEGTSESATPESGPGTSTEPS |
| | | EGSAPGAAEPEA |

TABLE 2c
Exemplary XTEN polypeptides

| Exemplary | SEQ ID | |
|---|---|---|
| Use | NO. | Amino Acid Sequence |
| C-terminal | 213 | PGSPAGSPTSTEEGTSESATPESGPGTSTEPSEGSAPGSPAGSPTSTEE |
| XTEN | | GTSTEPSEGSAPGTSTEPSEGSAPGTSESATPESGPGSEPATSGSETPG |
| | | SEPATSGSETPGSPAGSPTSTEEGTSESATPESGPGTSTEPSEGSAPGT |
| | | STEPSEGSAPGSPAGSPTSTEEGTSTEPSEGSAPGTSTEPSEGSAPGTS |
| | | ESATPESGPGTSTEPSEGSAPGTSESATPESGPGSEPATSGSETPGTST |
| | | EPSEGSAPGTSTEPSEGSAPGTSESATPESGPGTSESATPESGPGSPAG |
| | | SPTSTEEGTSESATPESGPGSEPATSGSETPGTSESATPESGPGTSTEP |
| | | SEGSAPGTSTEPSEGSAPGTSTEPSEGSAPGTSTEPSEGSAPGTSTEPS |
| | | EGSAPGTSTEPSEGSAPGSPAGSPTSTEEGTSTEPSEGSAPGTSESATP |
| | | ESGPGSEPATSGSETPGTSESATPESGPGSEPATSGSETPGTSESATPE |
| | | SGPGTSTEPSEGSAPGTSESATPESGPGSPAGSPTSTEEGSPAGSPTST |
| | | EEGSPAGSPTSTEEGTSESATPESGPGTSTEPSEGSAPGTSESATPESG |
| | | PGSEPATSGSETPGTSESATPESGPGSEPATSGSETPGTSESATPESGP |
| | | GTSTEPSEGSAPGSPAGSPTSTEEGTSESATPESGPGSEPATSGSETPG |
| | | TSESATPESGPGSPAGSPTSTEEGSPAGSPTSTEEGTSTEPSEGSAPGT |
| | | SESATPESGPGTSESATPESGPGftabTSESATPESGPGSEPATSGPTE |
| | | SGSEPATSGSETPGSPAGSPTSTEEGTSTEPSEGSAPGTESTPSEGSAP |
| | | GSEPATSGSETPGTSESATPESGPGTSTEPSEGSAPGEPEA |
| C-terminal | 214 | PGSPAGSPTSTEEGTSESATPESGPGTSTEPSEGSAPGSPAGSPTSTEE |
| XTEN | | GTSTEPSEGSAPGTSTEPSEGSAPGTSESATPESGPGSEPATSGSETPG |
| | | SEPATSGSETPGSPAGSPTSTEEGTSESATPESGPGTSTEPSEGSAPGT |
| | | STEPSEGSAPGSPAGSPTSTEEGTSTEPSEGSAPGTSTEPSEGSAPGTS |
| | | ESATPESGPGTSTEPSEGSAPGTSESATPESGPGSEPATSGSETPGTST |
| | | EPSEGSAPGTSTEPSEGSAPGTSESATPESGPGTSESATPESGPGSPAG |
| | | SPTSTEEGTSESATPESGPGSEPATSGSETPGTSESATPESGPGTSTEP |
| | | SEGSAPGTSTEPSEGSAPGTSTEPSEGSAPGTSTEPSEGSAPGTSTEPS |
| | | EGSAPGTSTEPSEGSAPGSPAGSPTSTEEGTSTEPSEGSAPGTSESATP |
| | | ESGPGSEPATSGSETPGTSESATPESGPGSEPATSGSETPGTSESATPE |
| | | SGPGTSTEPSEGSAPGTSESATPESGPGSPAGSPTSTEEGSPAGSPTST |
| | | EEGSPAGSPTSTEEGTSESATPESGPGTSTEPSEGSAPGTSESATPESG |
| | | PGSEPATSGSETPGTSESATPESGPGSEPATSGSETPGTSESATPESGP |
| | | GTSTEPSEGSAPGSPAGSPTSTEEGTSESATPESGPGSEPATSGSETPG |
| | | TSESATPESGPGSPAGSPTSTEEGSPAGSPTSTEEGTSTEPSEGSAPGT |
| | | SESATPESGPGTSESATPESGPGTSESATPESGPGSEPATSGPTESGSE |
| | | PATSGSETPGSPAGSPTSTEEGTSTEPSEGSAPGTSTEPSEGSAPGSEP |
| | | ATSGSETPGTSESATPESGPGTSTEPSEGSAPGEPEA |
| C-terminal | 215 | PGSPAGSPTSTEEGTSESATPESGPGTSTEPSEGSAPGSPAGSPTSTEE |
| XTEN | | GTSTEPSEGSAPGTSTEPSEGSAPGTSESATPESGPGSEPATSGSETPG |
| | | SEPATSGSETPGSPAGSPTSTEEGTSESATPESGPGTSTEPSEGSAPGT |
| | | STEPSEGSAPGSPAGSPTSTEEGTSTEPSEGSAPGTSTEPSEGSAPGTS |
| | | ESATPESGPGTSTEPSEGSAPGTSESATPESGPGSEPATSGSETPGTST |
| | | EPSEGSAPGTSTEPSEGSAPGTSESATPESGPGTSESATPESGPGSPAG |
| | | SPTSTEEGTSESATPESGPGSEPATSGSETPGTSESATPESGPGTSTEP |
| | | SEGSAPGTSTEPSEGSAPGTSTEPSEGSAPGTSTEPSEGSAPGTSTEPS |
| | | EGSAPGTSTEPSEGSAPGSPAGSPTSTEEGTSTEPSEGSAPGTSESATP |
| | | ESGPGSEPATSGSETPGTSESATPESGPGSEPATSGSETPGTSESATPE |
| | | SGPGTSTEPSEGSAPGTSESATPESGPGSPAGSPTSTEEGSPAGSPTST |
| | | EEGSPAGSPTSTEEGTSESATPESGPGTSTEPSEGSAPGTSESATPESG |
| | | PGSEPATSGSETPGTSESATPESGPGSEPATSGSETPGTSESATPESGP |
| | | GTSTEPSEGSAPGSPAGSPTSTEEGTSESATPESGPGSEPATSGSETPG |
| | | TSESATPESGPGSPAGSPTSTEEGSPAGSPTSTEEGTSTEPSEGSAPGT |
| | | SESATPESGPGTSESATPESGPGTSESATPESGPGSEPATSGSETPGSE |
| | | PATSGSETPGSPAGSPTSTEEGTSTEPSEGSAPGTESTPSEGSAPGSEP |
| | | ATSGSETPGTSESATPESGPGTSTEPSEGSAPGEPEA |
| N-terminal | 216 | ASSPAGSPTSTESGTSESATPESGPGTETEPSEGSAPGTSESATPESGP |
| XTEN | | GSEPATSGSETPGTSESATPESGPGSTPAESGSETPGTSESATPESGPG |
| | | TSTEPSEGSAPGSPAGSPTSTEEGTSESATPESGPGESPATSGSTPEGT |
| | | SESATPESGPGSPAGSPTSTEEGSPAGSPTSTEEGTSTEPSEGSAPGTS |
| | | ESATPESGPGTSESATPESGPGTSESATPESGPGSEPATSGSETPGSEP |
| | | ATSGSETPGSPAGSPTSTEEGTSTEPSEGSAPGTSTEPSEGSAPGGSAP |
| N-terminal | 217 | ASSPAGSPTSTESGTSESATPESGPGTSTEPSEGSAPGTSESATPESGP |
| XTEN | | GSEPATSGSETPGTSESATPESGPGSEPATSGSETPGTSESATPESGPG |
| | | TSTEPSEGSAPGSPAGSPTSTEEGTSESATPESGPGESPATSGSTPEGT |
| | | SESATPESGPGSPAGSPTSTEEGSPAGSPTSTEEGTSTEPSEGSAPGTS |
| | | ESATPESGPGTSESATPESGPGTSESATPESGPGSEPATSGSETPGSEP |
| | | ATSGSETPGSPAGSPTSTEEGTSTEPSEGSAPGTSTEPSEGSAPGGSAP |
| N-terminal | 218 | ASSPAGSPTSTESGTSESATPESGPGTSTEPSEGSAPGTSESATPESGP |
| XTEN | | GSEPATSGSETPGTSESATPESGPGSTPAESGSETPGTSESATPESGPG |
| | | TSTEPSEGSAPGSPAGSPTSTEEGTSESATPESGPGEEPATSGSTPEGT |
| | | SESATPESGPGSPAGSPTSTEEGSPAGSPTSTEEGTSTEPSEGSAPGTS |
| | | ESATPESGPGTSESATPESGPGTSESATPESGPGSEPATSGSETPGSEP |
| | | ATSGSETPGSPAGSPTSTEEGTSTEPSEGSAPGTSTEPSEGSAPGGSAP |
| N-terminal | 219 | ASSPAGSPTSTESGTSESATPESGPGTSTEPSEGSAPGTSESATPESGP |
| XTEN | | GSEPATSGSETPGTSESATPESGPGSTPAESGSETPGTSESATPESGPG |
| | | TSTEPSEGSAPGSPAGSPTSTEEGTSESATPESGPGSEPATSGSETPGT |
| | | SESATPESGPGSPAGSPTSTEEGSPAGSPTSTEEGTSTEPSEGSAPGTS |
| | | ESATPESGPGTSESATPESGPGTSESATPESGPGSEPATSGSETPGSEP |
| | | ATSGSETPGSPAGSPTSTEEGTSTEPSEGSAPGTSTEPSEGSAPGGSAP |
| C-terminal | 220 | PGSPAGSPTSTEEGTSESATPESGPGTSTEPSEGSAPGSPAGSPTSTEE |
| XTEN | | GTSTEPSEGSAPGTSTEPSEGSAPGTSESATPESGPGSEPATSGSETPG |
| | | SEPATSGSETPGSPAGSPTSTEEGTSESATPESGPGTSTEPSEGSAPGT |
| | | STEPSEGSAPGSPAGSPTSTEEGTSTEPSEGSAPGTSTEPSEGSAPGTS |
| | | ESATPESGPGTSTEPSEGSAPGTSESATPESGPGSEPATSGSETPGTST |
| | | EPSEGSAPGTSTEPSEGSAPGTSESATPESGPGTSESATPESGPGSPAG |
| | | SPTSTEEGTSESATPESGPGSEPATSGSETPGTSESATPESGPGTSTEP |
| | | SEGSAPGTSTEPSEGSAPGTSTEPSEGSAPGTSTEPSEGSAPGTSTEPS |
| | | EGSAPGTSTEPSEGSAPGSPAGSPTSTEEGTSTEPSEGSAPGTSESATP |
| | | ESGPGSEPATSGSETPGTSESATPESGPGSEPATSGSETPGTSESATPE |
| | | SGPGTSTEPSEGSAPGTSESATPESGPGSPAGSPTSTEEGSPAGSPTST |
| | | EEGSPAGSPTSTEEGTSESATPESGPGTSTEPSEGSAPGTSESATPESG |
| | | PGSEPATSGSETPGTSESATPESGPGSEPATSGSETPGTSESATPESGP |
| | | GTSTEPSEGSAPGSPAGSPTSTEEGTSESATPESGPGSEPATSGSETPG |
| | | TSESATPESGPGSPAGSPTSTEEGSPAGSPTSTEEGTSTEPSEGSAPGT |
| | | SESATPESGPGTSESATPESGPGTSESATPESGPGSEPATSGSETPGSE |
| | | PATSGSETPGSPAGSPTSTEEGTSTEPSEGSAPGTESTPSEGSAPGSEP |
| | | ATSGSETPGTSESATPESGPGTSTEPSEGSAPG |
| C-terminal | 221 | PGSPAGSPTSTEEGTSESATPESGPGSEPATSGSETPGTSESATPESGP |
| XTEN | | GTSTEPSEGSAPGTSTEPSEGSAPGTSTEPSEGSAPGTSTEPSEGSAPG |
| | | TSTEPSEGSAPGTSTEPSEGSAPGSPAGSPTSTEEGTSTEPSEGSAPGT |
| | | SESATPESGPGSEPATSGSETPGTSESATPESGPGSEPATSGSETPGTS |
| | | ESATPESGPGTSTEPSEGSAPGTSESATPESGPGSPAGSPTSTEEGSPA |
| | | GSPTSTEEGSPAGSPTSTEEGTSESATPESGPGTSTEPSEGSAPGTSES |
| | | ATPESGPGSEPATSGSETPGTSESATPESGPGSEPATSGSETPGTSESA |
| | | TPESGPGTSTEPSEGSAPGSPAGSPTSTEEGTSESATPESGPGSEPATS |
| | | GSETPGTSESATPESGPGSPAGSPTSTEEGSPAGSPTSTEEGTSTEPSE |
| | | GSAPGTSESATPESGPGTSESATPESGPGTSESATPESGPGSEPATSGS |
| | | ETPGSEPATSGSETPGSPAGSPTSTEEGTSTEPSEGSAPGTESTPSEGS |
| | | APGSEPATSGSETPGTSESATPESGPGTSTEPSEGSAPG |
| N-terminal | 222 | SAGSPGSPAGSPTSTEEGTSESATPESGPGTSTEPSEGSAPGSPAGSPT |
| XTEN | | STEEGTSTEPSEGSAPGTSTEPSEGSAPGTSESATPESGPGSTPAESGS |
| | | ETPGSEPATSGSETPGSPAGSPTSTEEGTSESATPESGPGTSTEPSEGS |
| | | APGTSTEPSEGSAPGSPAGSPTSTEEGTSTEPSEGSAPGTSTEPSEGSA |
| | | PGTSESATPESGPGTSTEPSEGSAPGTSESATPESGPGSEPATSGSTET |
| | | PGTSTEPSEGSAPGTSTEPSEGSAPGTSESATPESGPGTSESATPESGP |
| | | GSPAGSPTSTEEGTSESATPESGPGSEPATSGSETPGTSESATPESGPG |
| | | TSTEPSEGSAPGTSTEPSEGSAPGTSTEPSEGSAPGTSTEPSEGSAPGT |
| | | STEPSEGSAPGTSTEPSEGSAPGSPAGSPTSTEEGTSTEPSEGSAPGTS |
| | | ESATPESGPGSEPATSGSETPGTSESATPESGPGSEPATSGSETPGTSE |
| | | SATPESGPGTSTEPSEGSAPGTSESATPESGPGSPAGSPTSTEEGSPAG |
| | | SPTSTEEGSPAGSPTSTEEGTSESATPESGPGTSTEPSEGSAPGTSESA |
| | | TPESGPGSEPATSGSETPGTSESATPESGPGSEPATSGSETPGTSESAT |
| | | PESGPGTSTEPSEGSAPGSPAGSPTSTEEGTSESATPESGPGSEPATSG |
| | | SETPGTSESATPESGPGSPAGSPTSTEEGSPAGSPTSTEEGTSTEPSEG |
| | | SAPGTSESATPESGPGTSESATPESGPGTSESATPESGPGSEPATSGSE |
| | | TPGSEPATSGSETPGSPAGSPTSTEEGTSTEPSEGSAPGTSTEPSEGSA |
| | | PGSEPATSGSETPGTSESATPESGPGTSTEPSEGSAPGSPAGSPTSTEE |
| | | GTSESATPESGPGTSTEPSEGSAPGSPAGSPTSTEEGTSTEPSEGSAPG |
| | | TSTEPSEGSAPGTSESATPESGPGSEPATSGSETPGSEPATSGSETPGS |
| | | PAGSPTSTEEGTSESATPESGPGTSTEPSEGSAPGTSTEPSEGSAPGSP |
| | | AGSPTSTEEGTSTEPSEGSAPGTSTEPSEGSAPGTSESATPESGPGTST |
| | | EPSEGSAPGTSESATPESGPGSEPATSGSETPGTSTEPSEGSAPGTSTE |
| | | PSEGSAPGTSESATPESGPGTESAS |
| C-terminal | 223 | SAGSPGSPAGSPTSTEEGTSESATPESGPGTSTEPSEGSAPGSPAGSPT |
| XTEN | | STEEGTSTEPSEGSAPGTSTEPSEGSAPGTSESATPESGPGSEPATSGS |
| | | ETPGSEPATSGSETPGSPAGSPTSTEEGTSESATPESGPGTSTEPSEGS |
| | | APGTSTEPSEGSAPGSPAGSPTSTEEGTSTEPSEGSAPGTSTEPSEGSA |
| | | PGTSESATPESGPGTSTEPSEGSAPGTSESATPESGPGSEPATSGSETP |
| | | GTSTEPSEGSAPGTSTEPSEGSAPGTSESATPESGPGTSESATPESGPG |
| | | SPAGSPTSTEEGTSESATPESGPGSEPATSGSETPGTSESATPESGPGT |
| | | STEPSEGSAPGTSTEPSEGSAPGTSTEPSEGSAPGTSTEPSEGSAPGTS |
| | | TEPSEGSAPGTSTEPSEGSAPGSPAGSPTSTEEGTSTEPSEGSAPGTSE |
| | | SATPESGPGSEPATSGSETPGTSESATPESGPGSEPATSGSETPGTSES |
| | | ATPESGPGTSTEPSEGSAPGTSESATPESGPGSPAGSPTSTEEGSPAGS |
| | | PTSTEEGSPAGSPTSTEEGTSESATPESGPGTSTEPSEGSAPGTSESAT |
| | | PESGPGSEPATSGSETPGTSESATPESGPGSEPATSGSETPGTSESATP |
| | | ESGPGTSTEPSEGSAPGSPAGSPTSTEEGTSESATPESGPGSEPATSGS |
| | | ETPGTSESATPESGPGSPAGSPTSTEEGSPAGSPTSTEEGTSTEPSEGS |
| | | APGTSESATPESGPGTSESATPESGPGTSESATPESGPGSEPATSGSET |
| | | PGSEPATSGSETPGSPAGSPTSTEEGTSTEPSEGSAPGTSTEPSEGSAP |
| | | GSEPATSGSETPGTSESATPESGPGTSTEPSEGSAPGSPAGSPTSTEEG |
| | | TSESATPESGPGTSTEPSEGSAPGSPAGSPTSTEEGTSTEPSEGSAPGT |
| | | STEPSEGSAPGTSESATPESGPGSEPATSGSETPGSEPATSGSTETPGS |
| | | PAGSPTSTEEGTSESATPESGPGTSTEPSEGSAPGTSTEPSEGSAPGSP |
| | | AGSPTSTEEGTSTEPSEGSAPGTATESPEGSAPGTSESATPESGPGTST |
| | | EPSEGSAPGTSAESATPESGPGSEPATSGSETPGTSTEPSEGSAPGTST |
| | | EPSEGSAPGTSESATPESGPGTESAS |
| N-terminal | 224 | GSPAGSPTSTEEGTSESATPESGPGTSTEPSEGSAPGSPAGSPTSTEEG |
| XTEN | | TSTEPSEGSAPGTSTEPSEGSAPATSESATPESGPGSEPATSGSETPGS |
| | | EPATSGSETPGSPAGSPTSTEEGTSESASPESGPGTSTEPSEGSAPGTS |
| | | TEPSEGSAPGSPAGSPTSTEEGTSTEPSEGSAPGTSTEPSEGSAPGTSE |
| | | SATPESGPGTSTEPSEGSAPGTSESATPESGPGSEPATSGSETPGTSTE |
| | | PSEGSAPGTSTEPSEGSAPGTSESATPESGPGTSESATPESGPGSPAGS |
| | | PTSTEEGTSESATPESGPGSEPATSGSETPGTSESATPESGPGTSTEPS |
| | | EGSAPGTSTEPSEGSAPGTSTEPSEGSAPGTSTEPSEGSAPGTSTEPSE |
| | | GSAPGTSTEPSEGSAPGSPAGSPTSTEEGTSTEPSEGSAPGTSESATPE |
| | | SGPGSEPATSGSETPGTSESATPESGPGSEPATSGSETPGTSESATPES |
| | | GPGTSTEPSEGSAPGTSESATPESGPGSPAGSPTSTEEGSPAGSPTSTE |
| | | EGSPAGSPTSTEEGTSESATPESGPGTSTEPSEGSAPGTSESATPESGP |
| | | GSEPATSGSETPGTSESATPESGPGSEPATSGSETPGTSESATPESGPG |
| | | TSTEPSEGSAPGSPAGSPTSTEEGTSESATPESGPGSEPATSGSETPGT |
| | | SESATPESGPGSPAGSPTSTEEGSPAGSPTSTEEGTSTEPSEGSAPGTS |
| | | ESATPESGPGTSESATPESGPGTSESATPESGPGSEPATSGSETPGSEP |
| | | ATSGSETPGSPAGSPTSTEEGTSTEPSEGSAPGTSTEPSEGSAPGSEPA |
| | | TSGSETPGTSESATPESGPGTSTEPSEGSAP |
| N-terminal | 225 | GSPAGSPTSTEEGTSESATPESGPGTSTEPSEGSAPGSPAGSPTSTEEG |
| XTEN | | TSTEPSEGSAPGTSTEPSEGSAPGTSESATPESGPGSESATSGSETPGS |
| | | EPATSGSETPGSPAGSPTSTEEGTSESATPESGPGTSTEPSEGSAPGTS |
| | | TEPSEGSAPGSPAGSPTSTEEGTSTEPSEGSAPGTSTEPSEGSAPGTSE |
| | | SATPESGPGTSTEPSEGSAPGTSESATPESGPGSEPATSGSETPGTSTE |
| | | PSEGSAPGTSTEPSEGSAPGTSESATPESGPGTSESATPESGPGSPAGS |
| | | PTSTEEGTSESATPESGPGSEPATSGSETPGTSESATPESGPGTSTEPS |
| | | EGSAPGTSTEPSEGSAPGTSTEPSEGSAPGTSTEPSEGSAPGTSTEPSE |
| | | GSAPGTSTEPSEGSAPGSPAGSPTSTEEGTSTEPSEGSAPGTSESATPE |
| | | SGPGSEPATSGSETPGTSESATPESGPGSEPATSGSETPGTSESATPES |
| | | GPGTSTEPSEGSAPGTSESATPESGPGSPAGSPTSTEEGSPAGSPTSTE |
| | | EGSPAGSPTSTEEGTSESATPESGPGTSTEPSEGSAPGTSESATPESGP |
| | | GSEPATSGSETPGTSESATPESGPGSEPATSGSETPGTSESATPESGPG |
| | | TSTEPSEGSAPGSPAGSPTSTEEGTSESATPESGPGSEPATSGSETPGT |
| | | SESATPESGPGSPAGSPTSTEEGSPAGSPTSTEEGTSTEPSEGSAPGTS |
| | | ESATPESGPGTSESATPESGPGTSESATPESGPGSEPATSGSETPGSEP |
| | | ATSGSETPGSPAGSPTSTEEGTSTEPSEGSAPGTSTEPSEGSAPGSEPA |
| | | TSGSETPGTSESATPESGPGTSTEPSEGSAP |
| N-terminal | 226 | SPAGSPTSTESGTSESATPESGPGTSTEPSEGSAPGTSESATPESGPGS |
| XTEN (with | | EPATSGSETPGTSESATPESGPGSTPAESGSETPGTSESATPESGPGTS |
| His-tag) | | TEPSEGSAPGSPAGSPTSTEEGTSESATPESGPGSEPATSGSETPGTSE |
| | | SATPESGPGSPAGSPTSTEEGSPAGSPTSTEEGTSTEPSEGSAPGTSES |
| | | ATPESGPGTSESATPESGPGTSESATPESGPGSEPATSGSETPGSEPAT |
| | | SGSETPGSPAGSPTSTEEGTSTEPSEGSAPGTSTEPSEGSAPGGSAP |
| C-terminal | 227 | PGSPAGSPTSTEEGTSESATPESGPGSEPATSGSETPGTSESATPESGP |
| XTEN | | GTSTEPSEGSAPGTSTEPSEGSAPGTSTEPSEGSAPGTSTEPSEGSAPG |
| | | TSTEPSEGSAPGTSTEPSEGSAPGSPAGSPTSTEEGTSTEPSEGSAPGT |
| | | SESATPESGPGSEPATSGSETPGTSESATPESGPGSEPATSGSETPGTS |
| | | ESATPESGPGTSTEPSEGSAPGTSESATPESGPGSPAGSPTSTEEGSPA |
| | | GSPTSTEEGSPAGSPTSTEEGTSESATPESGPGTSTEPSEGSAPGTSES |
| | | ATPESGPGSEPATSGSETPGTSESATPESGPGSEPATSGSETPGTSESA |
| | | TPESGPGTSTEPSEGSAPGSPAGSPTSTEEGTSESATPESGPGSEPATS |
| | | GSETPGTSESATPESGPGSPAGSPTSTEEGSPAGSPTSTEEGTSTEPSE |
| | | GSAPGTSESATPESGPGTSESATPESGPGTSESATPESGPGSEPATSGS |
| | | ETPGSEPATSGSETPGSPAGSPTSTEEGTSTEPSEGSAPGTESTPSEGS |
| | | APGSEPATSGSETPGTSESATPESGPGTSTEPSEGSAPGEPEA |
| C-terminal | 228 | TPESGPGTSESATPESGPGSPAGSPTSTEEGTSESATPESGPGSEPATS |
| XTEN | | GSETPGTSESATPESGPGTSTEPSEGSAPGTSTEPSEGSAPGTSTEPSE |
| | | GSAPGTSTEPSEGSAPGTSTEPSEGSAPGTSTEPSEGSAPGSPAGSPTS |
| | | TEEGTSTEPSEGSAPGTSESATPESGPGSEPATSGSETPGTSESATPES |
| | | GPGSEPATSGSETPGTSESATPESGPGTSTEPSEGSAPGTSESATPESG |
| | | PGSPAGSPTSTEEGSPAGSPTSTEEGSPAGSPTSTEEGTSESATPESGP |
| | | GTSTEPSEGSAPGTSESATPESGPGSEPATSGSETPGTSESATPESGPG |
| | | SEPATSGSETPGTSESATPESGPGTSTEPSEGSAPGSPAGSPTSTEEGT |
| | | SESATPESGPGSEPATSGSETPGTSESATPESGPGSPAGSPTSTEEGSP |
| | | AGSPTSTEEGTSTEPSEGSAPGTSESATPESGPGTSESATPESGPGTSE |
| | | SATPESGPGSEPATSGSETPGSESATSGSETPGSPAGSPTSTEEGTSTE |
| | | PSEGSAPGTSTEPSEGSAPGSEPATSGSETPGTSESA |
| C-terminal | 229 | GTSTEPSEGSAPGTSESATPESGPGSEPATSGSETPGSEPATSGSETPG |
| XTEN | | SPAGSPTSTEEGTSESATPESGPGTSTEPSEGSAPGTSTEPSEGSAPGS |
| | | PAGSPTSTEEGTSTEPSEGSAPGTSTEPSEGSAPGTSESATPESGPGTS |
| | | TEPSEGSAPGTSESATPESGPGSEPATSGSETPGTSTEPSEGSAPGTST |
| | | EPSEGSAPGTSESATPESGPGTSESATPESGPGSPAGSPTSTEEGTSES |
| | | ATPESGPGSEPATSGSETPGTSESATPESGPGTSTEPSEGSAPGTSTEP |
| | | SEGSAPGTSTEPSEGSAPGTSTEPSEGSAPGTSTEPSEGSAPGTSTEPS |
| | | EGSAPGSPAGSPTSTEEGTSTEPSEGSAPGTSESATPESGPGSEPATS |
| | | GSETPGTSESATPESGPGSEPATSGSETPGTSESATPESGPGTSTEPSE |
| | | GSAPGTSESASPESGPGSPAGSPTSTEEGSPAGSPTSTEEGSPAGSPTS |
| | | TEEGTSESATPESGPGTSTEPSEGSAPGTSESATPESGPGSEPATSGSE |
| | | TPGTSESATPESGPGSEPATSGSETPGTSESATPESGP |
| C-terminal | 230 | GSETPGSPAGSPTSTEEGTSESATPESGPGTSTEPSEGSAPGTSTEPSE |
| XTEN | | GSAPGSPAGSPTSTEEGTSTEPSEGSAPGTSTEPSEGSAPGTSESATPE |
| | | SGPGTSTEPSEGSAPGTSESATPESGPGSEPATSGSETPGTSTEPSEGS |
| | | APGTSTEPSEGSAPGTSESATPESGPGTSESATPESGPGSPAGSPTSTE |
| | | EGTSESATPESGPGSEPATSGSETPGTSESATPESGPGTSTEPSEGSAP |
| | | GTSTEPSEGSAPGTSTEPSEGSAPGTSTEPSEGSAPGTSTEPSEGSAPG |
| | | TSTEPSEGSAPGSPAGSPTSTEEGTSTEPSEGSAPGTSESATPESGPGS |
| | | EPATSGSETPGTSESATPESGPGSEPATSGSETPGTSESATPESGPGTS |
| | | TEPSEGSAPGTSESATPESGPGSPAGSPTSTEEGSPAGSPTSTEEGSPA |
| | | GSPTSTEEGTSESATPESGPGTSTEPSEGSAPGTSESATPESGPGSEPA |
| | | TSGSTETGTSESATPESGPGSEPATSGSETPGTSESATPESGPGTSTEP |
| | | SEGSAPGSPAGSPTSTEEGTSESATPESGPGSEPATS |
| C-terminal | 231 | EGSAPGTSTEPSEGSAPGTSESATPESGPGTSTEPSEGSAPGTSESATP |
| XTEN | | ESGPGSEPATSGSETPGTSTEPSEGSAPGTSTEPSEGSAPGTSESATPE |
| | | SGPGTSESATPESGPGSPAGSPTSTEEGTSESATPESGPGSEPATSGSE |
| | | TPGTSESATPESGPGTSTEPSEGSAPGTSTEPSEGSAPGTSTEPSEGSA |
| | | PGTSTEPSEGSAPGTSTEPSEGSAPGTSTEPSEGSAPGSPAGSPTSTEE |
| | | GTSTEPSEGSAPGTSESATPESGPGSEPATSGSETPGTSESATPESGPG |
| | | SEPATSGSETPGTSESATPESGPGTSTEPSEGSAPGTSESATPESGPGS |
| | | PAGSPTSTEEGSPAGSPTSTEEGSPAGSPTSTEEGTSESATPESGPGTS |
| | | TEPSEGSAPGTSESATPESGPGSEPATSGSETPGTSESATPESGPGSEP |
| | | ATSGSETPGTSESASPESGPGTSTEPSEGSAPGSPAGSPTSTEEGTSES |
| | | ATPESGPGSEPATSGSETPGTSESATPESGPGSPAGSPTSTEEGSPAGS |
| | | PTSTEEGTSTEPSEGSAPGTSESATPESGPGTSESAT |
| N-terminal | 232 | ASSPAGSPTSTESGTSESATPESGPGTSTEPSEGSAPGTSESATPESGP |
| | | GSEPATSGSETPGTSESATPESGPGSTPAESGSETPGTSESATPESGPG |
| | | TSTEPSEGSAPGSPAGSPTSTEEGTSESATPESGPGSEPATSGSETPGT |
| | | SESATPESGPGSPAGSPTSTEEGSPAGSPTSTEEGTSTEPSEGSAPGTS |
| | | ESATPESGPGTSESATPESGPGTSESATPESGPGSEPATSGSETPGSEP |
| | | ATSGSETPGSPAGSPTSTEEGTSTEPSEGSAPGTSTEPSEGSAPGGSAP |

Additional examples of XTEN sequences that can be used according to the present disclosure are disclosed in U.S. Patent Publication Nos. 2010/0239554 A1, 2010/0323956 A1, 2011/0046060 A1, 2011/0046061 A1, 2011/0077199 A1, 2011/0172146 A1, 2018/0244736 A1, 2018/0346952 A1, and 2019/0153115 A1; U.S. Pat. Nos. 8,673,860, 9,371,369, 9,926,351, 9,249,211, and 9,976,166; and International Patent Publication Nos. WO 2010/091122 A1, WO 2010/144502 A2, WO 2010/144508 A1, WO 2011/028228 A1, WO 2011/028229 A1, WO 2011/028344 A2, WO 2014/011819 A2, WO 2015/023891, WO 2016/077505 A2, WO 2017/040344 A2, and WO 2019/126576 A1.

In general, XTEN are polypeptides with non-naturally occurring, substantially non-repetitive sequences having a low degree or no secondary or tertiary structure under physiologic conditions, as well as additional properties described in the paragraphs that follow. XTEN can have at least (about) 100, at least (about) 150, at least (about) 200, at least (about) 300, at least (about) 400, at least (about) 500, at least (about) 600, at least (about) 700, at least (about) 800, at least (about) 900, at least (about) 1,000 amino acids, or a range between any of the foregoing. As used herein, XTEN specifically excludes whole antibodies or antibody fragments (e.g. single-chain antibodies and Fc fragments). XTEN polypeptides have utility as fusion partners in that they serve in various roles, conferring certain desirable properties when linked to a composition comprising, for example, one or more biologically active moieties (such as one described herein). The resulting compositions have enhanced properties, such as enhanced pharmacokinetic, physicochemical, pharmacologic, and improved toxicological and pharmaceutical properties compared to the corresponding one or more biologically active moieties not linked to XTEN, making them useful in the treatment of certain conditions for which the one or more biologically active moieties are known in the art to be used.

The unstructured characteristic and physicochemical properties of the XTEN result, in part, from the overall amino acid composition that is disproportionately limited to 4-6 types of hydrophilic amino acids, the sequence of the amino acids in a quantifiable, substantially non-repetitive design, and from the resulting length of the XTEN polypeptide. In an advantageous feature common to XTEN but uncommon to native polypeptides, the properties of XTEN disclosed herein may not be tied to an absolute primary amino acid sequence, as evidenced by the diversity of the exemplary sequences of Tables 2b-2c that, within varying ranges of length, possess similar properties and confer enhanced properties on the compositions to which they are linked, many of which are documented in the Examples. Indeed, it is specifically contemplated that the compositions of the disclosure not be limited to those XTEN specifically enumerated in Tables 8 or 10, but, rather, the embodiments at least include sequences having at least 90%, 91%, 92%, 93%, 94%, 95%, 96%, 97%, 98%, or 99% sequence identity, when optimally aligned, to the sequences of Tables 2b-2c as they exhibit the properties of XTEN described herein. It has been established that such XTEN have properties more like non-proteinaceous, hydrophilic polymers (such as polyethylene glycol, or “PEG”) than they do proteins. The XTEN of the present disclosure exhibit one or more of the following advantageous properties: defined and uniform length (for a given sequence), conformational flexibility, reduced or lack of secondary structure, high degree of random coil formation, high degree of aqueous solubility, high degree of protease resistance, low immunogenicity, low binding to mammalian receptors, a defined degree of charge, and increased hydrodynamic (or Stokes) radii; properties that are similar to certain hydrophilic polymers (e.g., polyethylene glycol) that make them particularly useful as fusion partners.

XTEN, as described herein, are designed to behave like denatured peptide sequences under physiological conditions, despite the extended length of the polymer. “Denatured” describes the state of a peptide in solution that is characterized by a large conformational freedom of the peptide backbone. Most peptides and proteins adopt a denatured conformation in the presence of high concentrations of denaturants or at elevated temperature. Peptides in denatured conformation have, for example, characteristic circular dichroism (CD) spectra and are characterized by a lack of long-range interactions as determined by NMR. “Denatured conformation” and “unstructured conformation” are used synonymously herein. In some embodiments, the disclosure provides compositions that comprise XTEN sequences that, under physiologic conditions, resemble denatured sequences that are substantially devoid of secondary structure under physiologic conditions. “Substantially devoid,” as used in this context, means that at least about 80%, or about 90%, or about 95%, or about 97%, or at least about 99% of the XTEN amino acid residues of the XTEN sequence do not contribute to secondary structure, as measured or determined by the methods described herein, including algorithms or spectrophotometric assays.

A variety of well-established methods and assays are known in the art for determining and confirming the physicochemical properties of the subject XTEN and the subject polypeptide compositions into which they are incorporated. Such properties include but are not limited to secondary or tertiary structure, solubility, protein aggregation, stability, absolute and apparent molecular weight, purity and uniformity, melting properties, contamination and water content. The methods to measure such properties include analytical centrifugation, EPR, HPLC-ion exchange, HPLC-size exclusion chromatography (SEC), HPLC-reverse phase, light scattering, capillary electrophoresis, circular dichroism, differential scanning calorimetry, fluorescence, HPLC-ion exchange, HPLC-size exclusion, IR, NMR, Raman spectroscopy, refractometry, and UV/Visible spectroscopy. In particular, secondary structure can be measured spectrophotometrically, e.g., by circular dichroism spectroscopy in the “far-UV” spectral region (190-250 nm). Secondary structure elements, such as alpha-helix and beta-sheet, each give rise to a characteristic shape and magnitude of CD spectra, as does the lack of these structure elements. Secondary structure can also be predicted for a polypeptide sequence via certain computer programs or algorithms, such as the well-known Chou-Fasman algorithm (Chou, P. Y., et al. (1974) Biochemistry, 13: 222-45) and the Garnier-Osguthorpe-Robson algorithm (“GOR IV algorithm”) (Gamier J, Gibrat J F, Robson B. (1996), GOR method for predicting protein secondary structure from amino acid sequence. Methods Enzymol 266:540-553), as described in US Patent Application Publication No. 20030228309A1. For a given sequence, the algorithms can predict whether there exists some or no secondary structure at all, expressed as the total and/or percentage of residues of the sequence that form, for example, alpha-helices or beta-sheets or the percentage of residues of the sequence predicted to result in random coil formation (which lacks secondary structure). Polypeptide sequences can be analyzed using the Chou-Fasman algorithm using sites on the world wide web at, for example, fasta.bioch.virginia.edu/fasta_www2/fasta_www.cgi?rm=misc1 and the GOR IV algorithm at npsa-pbil.ibcp.fr/cgi-bin/npsa_automat.pl?page=npsa_gor4.html (both accessed on Dec. 8, 2017). Random coil can be determined by a variety of methods, including by using intrinsic viscosity measurements, which scale with chain length in a conformation-dependent way (Tanford, C., Kawahara, K. & Lapanje, S. (1966) J. Biol. Chem. 241, 1921-1923), as well as by size-exclusion chromatography (Squire, P. G., Calculation of hydrodynamic parameters of random coil polymers from size exclusion chromatography and comparison with parameters by conventional methods. Journal of Chromatography, 1981, 5,433-442). Additional methods are disclosed in Arnau, et al., Prot Expr and Purif (2006) 48, 1-13.

In some embodiments of the present disclosure, the activatable therapeutic agent is an activatable antibody (AA) composition, where the masking moiety (MM) refers to an amino acid sequence coupled to an antibody or antibody fragment (AB) and positioned such that it reduces the ability of the AB to bind its designated binding target by specifically binding to the antigen-binding domain of the AB (such as the complementarity-determining region(s) (CDR(s)). Such binding can be non-covalent. In some embodiments, the activatable antibody composition can be prevented from binding to the designated binding target by binding the MM to an N- or C-terminus of the activatable antibody composition.

Alternatively, the MM may not specifically bind the AB, but rather interfere with AB-target binding through non-specific interactions such as steric hindrance. For example, the MM may be positioned in the uncleaved activatable antibody composition such that the tertiary or quaternary structure of the activatable antibody allows the MM to mask the AB through charge-based interaction, thereby holding the MM in place to interfere with target access to the AB. The masking moiety (MM) can interfere or/and inhibit binding of the antibody or antibody fragment (AB) to the target allosterically or sterically.

When the antibody or antibody fragment (AB) is modified with a MM and is in the presence of the target, specific binding of the AB to its target can be reduced or inhibited, as compared to the specific binding of the AB, not modified with an MM, to the target. A dissociation constant (K_{d}) of the AB modified with a MM towards the AB's target can be generally greater than a corresponding K_{d} of the AB, not modified with a MM, towards the target. Conversely, a binding affinity of the AB modified with a MM towards the target can be generally lower than a binding affinity of the AB, not modified with a MM, towards the target. In some embodiments, the masking moiety (MM) of the activatable antibody composition can have an equilibrium dissociation constant (K_{d}) for binding to the antibody or a fragment thereof which is greater than the equilibrium dissociation of the antibody or the fragment thereof for binding to its designated binding target (near or at a diseased site in a subject).

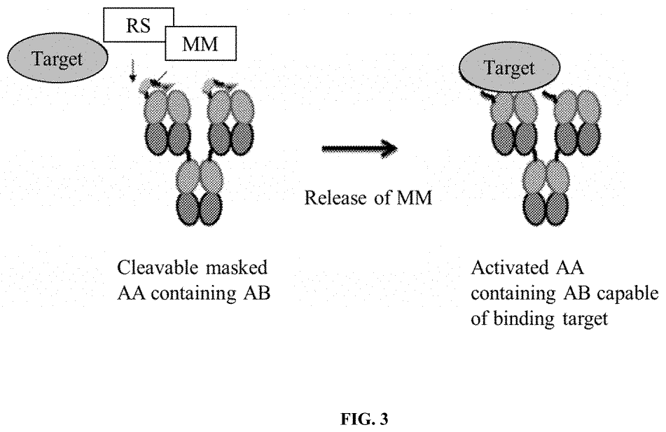

When the antibody or antibody fragment (AB) is modified with a release segment (RS) and a masking moiety (MM) and is in the presence of the target but not sufficient protease or protease activity to cleave the RS, specific binding of the modified AB to the target can be generally reduced or inhibited, as compared to the specific binding of the AB modified with a RS and a MM in the presence of the target and sufficient protease or protease activity to cleave the RS. For example, when the modified antibody is an activatable antibody composition and comprises a release segment (RS), the AB can be unmasked upon cleavage of the RS, in the presence of protease, preferably a disease-specific protease. Thus, the MM is one that when the activatable antibody composition is uncleaved provides for masking of the AB from target binding, but does not substantially or significantly interfere or compete for binding of the target to the AB when the activatable antibody composition is in the cleaved conformation. A schematic of an exemplary activatable antibody (AA) composition is provided in FIG. 3. As illustrated, the release segment (RS) is positioned such that in a cleaved (or relatively active state) and in the presence of a target, the antibody or antibody fragment (AB) binds a target, while in an uncleaved (or relatively inactive state) in the presence of the target, specific binding of the AB to its target is reduced or inhibited. The specific binding of the antibody or antibody fragment (AB) to its target can be reduced due to the due to the inhibition or masking of the AB's ability to specifically bind its target by the masking moiety (MM).

In some embodiments of the activatable antibody compositions, where an antibody or antibody fragment (AB) is capable of specifically binding its designated binding target, a coupling of the masking moiety (MM) to the antibody or antibody fragment (AB) can reduce the ability of the AB to bind its designated binding target as compared to the ability of the AB not coupled to the MM to bind the designated binding target (for example, when assayed in vitro using a target displacement assay). Such coupling of the MM to the AB can reduce the ability of the AB to bind its designated binding target for a duration.

The masking moiety (MM) can be provided in a variety of different forms. In certain embodiments, the MM can be selected to be a known binding partner of the antibody or antibody fragment (AB), provided that the MM binds the AB with less affinity and/or avidity than the target protein to which the AB is designed to bind following cleavage of the release segment (RS) so as to reduce interference of MM in target-AB binding Stated differently, as discussed above, the MM is one that masks the AB from target binding when the activatable antibody composition is uncleaved, but does not substantially or significantly interfere or compete for binding for target when the activatable antibody composition is in the cleaved conformation. In a specific embodiment, the AB and MM do not contain the amino acid sequences of a naturally-occurring binding partner pair, such that at least one of the AB and MM does not have the amino acid sequence of a member of a naturally occurring binding partner. The masking moiety (MM) may not comprise more than 50% amino acid sequence identity to a natural binding partner of the antibody or antibody fragment (AB). The masking moiety (MM) can comprise a consensus sequence specific for binding to a class of antibodies against a designated binding target (e.g., diseased target). The MM can be a polypeptide of no more than 40 (e.g., from 2 to 40) amino acids in length. The MM can be coupled to the activatable antibody composition by covalent binding.

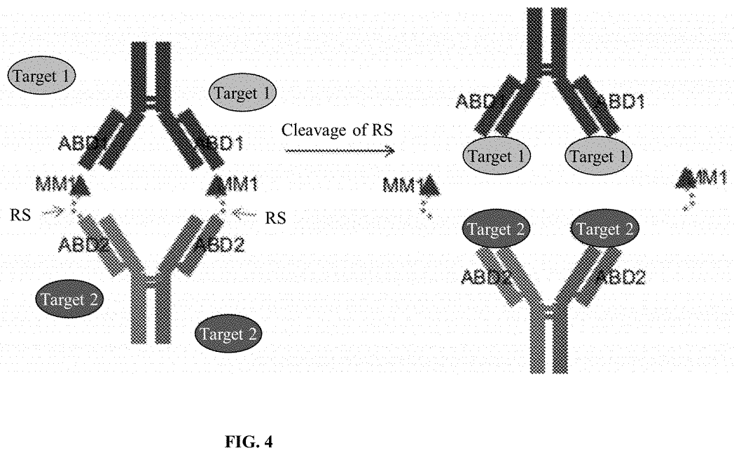

In some embodiments, the present disclosure provides for an activatable antibody complex (AAC) composition (as illustrated in FIG. 4) comprising: (1) two antibodies or antibody fragments (AB1 and AB2), each capable of specifically binding its designated binding target, (2) at least one masking moiety (MM) coupled to either AB1 or AB2, capable of inhibiting the specific binding of AB1 and AB2 to their designated binding target(s), and (3) at least one release segment (RS) coupled to either AB1 or AB2, capable of being specifically cleaved by a protease whereby activating the AAC composition. In some embodiments, when the AAC is in an uncleaved state, the MM can inhibit the specific binding of AB1 and AB2 to their designated binding target(s) and when the AAC is in a cleaved state, the MM does not inhibit the specific binding of AB1 and AB2 to their designated binding targets. The two ABs can bind different targets, or different epitopes on the same target.

In some embodiments, the MM does not inhibit cellular entry of the activatable antibody composition.

In some embodiments, the masking moiety (MM) can comprise an anti-albumin domain, such as a single domain antibody (sdAb) anti-albumin domain. In some embodiments, the anti-albumin domain can comprise non-CDR loops, CDR loops, or any combination thereof. In some embodiments, the anti-albumin domain can comprise both non-CDR loops and CDR loops. The non-CDR loops can be capable of binding to one or more antibody or antibody fragment (AB) (for example, and not limited to, the CDRs of the AB) of an activatable antibody (AA) composition, thereby masking the AB (at least in some cases) by inhibiting or reducing the ability of the AB to bind to its designated target(s). The CDR loops can be capable of binding albumin (e.g., human serum albumin), thereby (at least in some cases) masking the AB in the activatable antibody (AA) composition from binding to its designated target(s) via steric or allosteric hindrance and/or conferring half-life extension for the AA composition. In some embodiments, the non-CDR loops can be engineered into different position of the anti-albumin sdAb domain. In some embodiments, the MM can (1) inhibit or reduce the ability of the AB to bind to its designated target(s) via (1a) specific binding to the target recognition region of the AB and/or (1b) steric masking of target recognition region of the AB, and/or the MM can (2) confer half-life extension for the AA containing the AB via binding to albumin. The MM can be coupled (directly or indirectly) to the activatable antibody composition by covalent binding.

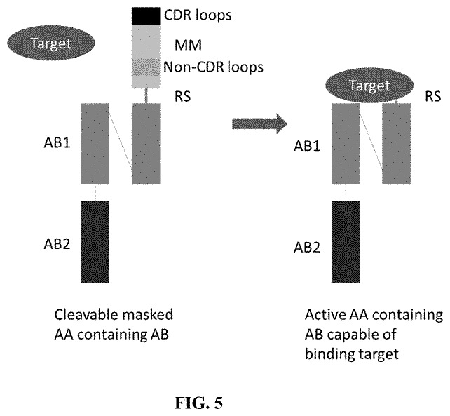

As illustrated in the schematic shown in FIG. 5, an exemplary activatable antibody complex (AAC) composition can comprise: (1) at least two antibodies or antibody fragments (AB1 and AB2), each capable of specifically binding their designated binding target(s), (2) at least one masking moiety (MM) coupled to AB1 or AB2, capable of inhibiting the specific binding of AB1 or AB2 to their designated binding target(s), and (3) at least one release segment (RS) coupled to AB1 or AB2, capable of being specifically cleaved by a protease whereby activating the activatable antibody complex (AAC) composition. In some embodiments, when the AA is in an uncleaved state, the MM can inhibit the specific binding of AB1 or AB2 to their designated binding target(s), and when the activatable antibody complex (AAC) composition is in a cleaved state, the MM does not inhibit the specific binding of AB1 or AB2 to their designated binding target(s). In some embodiments, the masking moiety (MM) can be coupled to both AB1 and AB2 via two separate release segments (RS). In other words, the MM can be placed between AB1 and AB2, coupled either to the C end of AB1 and the N end of AB2, or coupled to the N end of AB1 and the C end of AB2.

In some embodiments of the present disclosure, the activatable therapeutic agent is an activatable antibody (AA) composition, where the masking moiety (MM) refers to an amino acid sequence coupled to an antibody or antibody fragment (AB) (for example, but not limited to, an scFv, an sdAb, or a fragment thereof) and positioned such that it reduces the ability of the AB to dimerize with another antibody or antibody fragment, preventing the formation of an antibody or an antibody fragment capable of binding to target. Such binding can be non-covalent. In some embodiments, the activatable antibody composition can be prevented from binding to the designated binding target by binding the MM to an N- or C-terminus of the activatable antibody composition.

When the antibody or antibody fragment (AB) is modified with a MM and is in the presence of the target, specific binding of the AB to its dimerization partner can be reduced or inhibited, as compared to the specific binding of the AB, not modified with an MM, to its dimerization partner. A dissociation constant (K_{d}) of the AB modified with a MM towards its dimerization partner can be generally greater than a corresponding K_{d} of the AB, not modified with a MM, towards its dimerization partner. Conversely, a binding affinity of the AB modified with a MM towards its dimerization partner can be generally lower than a binding affinity of the AB, not modified with a MM, towards its dimerization partner. In some embodiments, the masking moiety (MM) of the activatable antibody composition can have an equilibrium dissociation constant (K_{d}) for binding to the antibody or a fragment thereof which is greater than the equilibrium dissociation of the antibody or the fragment thereof for binding to its designated dimerization partner.

When the antibody or antibody fragment (AB) is modified with a release segment (RS) and a masking moiety (MM) and is in the presence of the target but not sufficient protease or protease activity to cleave the RS, specific ability of the modified AB to dimerize with another antibody or antibody fragment and the resulting ability of the dimer to bind to its designated binding target(s) can be generally reduced or inhibited, as compared to the specific dimerization ability of the AB modified with a RS and a MM and the subsequent ability of the dimer to bind to its designated binding target(s) in the presence of the target and sufficient protease or protease activity to cleave the RS. For example, when the modified antibody is an activatable antibody composition and comprises a release segment (RS), the AB can be unmasked upon cleavage of the RS, in the presence of protease, preferably a disease-specific protease. Thus, the MM is one that when the activatable antibody composition is uncleaved provides for masking of the AB from dimerization with another AB and for reduction or inhibition of binding of the resulting dimer to its designated binding target(s), but does not substantially or significantly interfere or compete for dimerization to another AB and for reduction or inhibition of binding of the resulting dimer to its designated binding target(s) when the activatable antibody composition is in the cleaved conformation.

The masking moiety can be provided in different forms. In some embodiments, the masking domain can be an inhibitory antibody or antibody fragment (IAB; for example, but not limited to, a VL or VH domain), provided that the MM binds the AB with less affinity and/or avidity than the dimerization partner with which AB is designed to dimerize following cleavage of the release segment (RS) so as to reduce interference of MM in AB-AB dimerization. Stated differently, as discussed above, the MM is one that masks the AB from dimerization to another AB when the activatable antibody composition is uncleaved, but does not substantially or significantly interfere or compete for dimerization with another AB when the activatable antibody composition is in the cleaved conformation. The MM can be coupled to the activatable antibody composition by covalent binding.

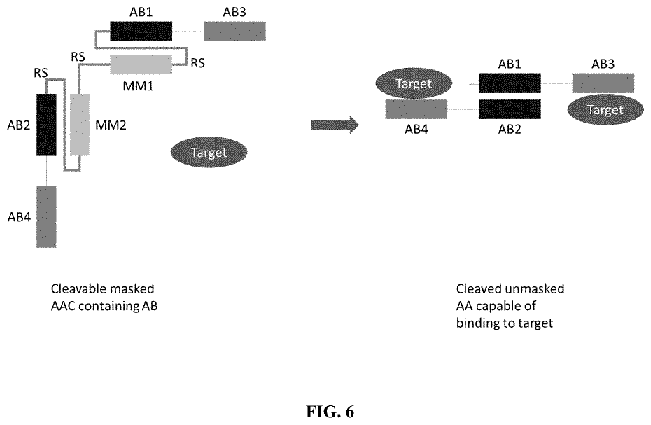

In some embodiments, the present disclosure provides for an activatable antibody complex (AAC) composition (as illustrated in FIG. 6) comprising: (1) two antibodies or antibody fragments (AB1 and AB2) (2) two masking moieties (MM) coupled to one each to AB1 and AB2, capable of reducing or inhibiting the specific dimerization of AB1 and AB2 and subsequent binding of AB1-AB2 complex to their designated binding target(s), (3) at least three release segments (RS) coupled to AB1, AB2 and MMs capable of being specifically cleaved by a protease whereby activating the AAC composition, (4) at least one additional antibody or antibody fragment (AB3 and/or AB4; for example, but not limited to, an scFv or an sdAb), coupled to AB1 and/or AB2. In some embodiments, when the AAC is in an uncleaved state, the MM can inhibit or reduce the specific dimerization of AB1 and AB2 and subsequently inhibit or reduce the binding of the resulting AB1-AB2 dimer to its designated binding target(s) and when the AAC is in a cleaved state, the MM does not reduce or inhibit the specific dimerization of AB1 and AB2 and does not reduce or inhibit the subsequent binding of the AB1-AB2 dimer to its designated binding target(s). When more than one additional AB is coupled to AB1 and/or AB2, the additional ABs can bind the same target or different targets.

In some embodiments, the MM can comprise a coiled-coil domain, for example, but not limited to, (1) high affinity parallel heterodimeric leucine zipper coiled-coil domain, containing or devoid of cysteines, (2) low affinity parallel heterodimeric coiled-coil leucine zipper domain, containing or devoid of cysteines, (3) disulfide-linked covalent coiled-coil domain, (4) antiparallel heterodimeric leucine zipper coiled-coil domain, (5) helix-turn-helix homodimeric leucine zipper coiled coil domain. The MM can be coupled (directly or indirectly) to the activatable antibody composition by covalent binding. In some embodiments, the MM can reduce or inhibit the binding of AB to its intended target(s) via steric or allosteric hindrance.

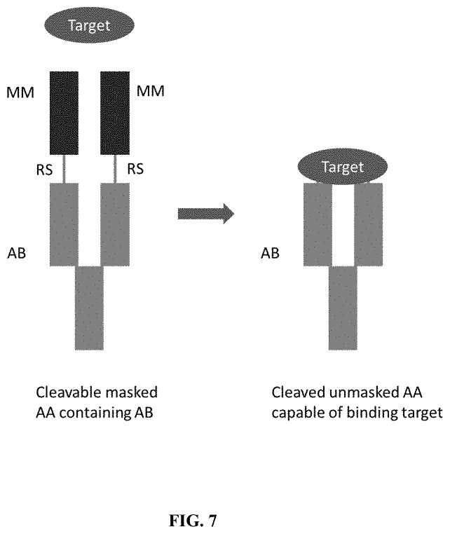

In some embodiments, the present disclosure provides for an activatable antibody complex (AAC) composition (as illustrated in FIG. 7) comprising: (1) at least one antibody or antibody fragment (AB), (2) at least one masking moiety (MM) coupled to AB, capable of inhibiting the specific binding of AB to its designated binding target, and (3) at least one release segment (RS) coupled to AB, capable of being specifically cleaved by a protease whereby activating the AAC composition. In some embodiments, when the AAC is in an uncleaved state, the MM can reduce or inhibit the specific binding of AB to its designated binding target(s) and when the AAC is in a cleaved state, the MM does not reduce or inhibit the specific binding of AB to its designated binding target(s).

In some embodiments, the activatable therapeutic agent may incorporate a cleavage sequence as described herein, and/or be administered to a patient who is identified as being a likely responder to the therapeutic agent based on the identification of a peptide biomarker in a biological sample from the subject (as described further herein).

### Biologically Active Moieties (BM)

In some embodiments of the therapeutic agent (or the activatable therapeutic agent, or the non-natural, activatable therapeutic agent), the biologically active moiety (BM) can comprise a biologically active peptide (BP). The biologically active peptide (BP) can comprise an antibody, a cytokine, a cell receptor, or a fragment thereof. The biologically active polypeptide (BP) can comprise a binding moiety having a binding affinity for a target cell marker on a target tissue or cell. The target cell marker can be an effector cell antigen expressed on a surface of an effector cell. The binding moiety can be an antibody. The antibody can be selected from the group consisting of Fv, Fab, Fab′, Fab′-SH, nanobody (also known as single domain antibody or V_{HH}), linear antibody, and single-chain variable fragment (scFv).

In some embodiments of the therapeutic agent (or the activatable therapeutic agent, or the non-natural, activatable therapeutic agent), where the binding moiety can be a first binding moiety, and wherein the target cell marker can be a first target cell marker, the biologically active polypeptide (BP) can further comprise a second binding moiety linked, directly or indirectly to the first binding moiety. The second binding moiety can have a binding affinity for a second target cell marker on the target tissue or cell. The second target cell marker can be a marker on a tumor cell or a cancer cell. The second binding moiety can be an antibody. The second binding moiety can be an antibody selected from the group consisting of Fv, Fab, Fab′, Fab′-SH, nanobody (also known as single domain antibody or V_{HH}), linear antibody, and single-chain variable fragment (scFv).

In some embodiments as disclosed herein, a biologically active moiety (BM) or a biologically active peptide (BP) can exhibit a binding specificity to a given target (or a given number of targets) or/and another desired biological characteristic, when used in vivo or when utilized in an in vitro assay. For example, the BM or BP can be an agonist, a receptor, a ligand, an antagonist, an enzyme, an antibody (e.g., mono- or bi-specific), or a hormone. Of particular interest are BM or BP used, or known to be useful, for a disease or disorder where the native BM or BP have a relatively short terminal half-life and for which an enhancement of a pharmacokinetic parameter (which optionally could be released from a conjugate or a fusion polypeptide by cleavage of a spacer sequence) would permit less frequent dosing or an enhanced pharmacologic effect. Also of interest are BM or BP that have a relatively narrow therapeutic window between the minimum effective dose or blood concentration (Cₘᵢₙ) and the maximum tolerated dose or blood concentration (Cₘₐₓ). In such cases, the linking of the BM or BP within a conjugate or a fusion polypeptide comprising a select masking moiety, such as XTEN, can result in an improvement in these properties, making them more useful as therapeutic or preventive agents compared to the BM or BP not linked to a masking moiety, such as XTEN. The BM or BP encompassed by the inventive compositions described herein can have utility in the treatment in various therapeutic or disease categories, including but not limited to glucose and insulin disorders, metabolic disorders, cardiovascular diseases, coagulation and bleeding disorders, growth disorders or conditions, endocrine disorders, eye diseases, kidney diseases, liver diseases, tumorigenic conditions, inflammatory conditions, autoimmune conditions, etc.

In some embodiments of the compositions disclosed herein, where the biologically active moiety is a biologically active peptide (BP), the BP can comprise a peptide sequence that exhibits at least (about) 80% sequence identity (e.g., at least (about) 81%, at least (about) 82%, at least (about) 83%, at least (about) 84%, at least (about) 85%, at least (about) 86%, at least (about) 87%, at least (about) 88%, at least (about) 89%, at least (about) 90%, at least (about) 91%, at least (about) 92%, at least (about) 93%, at least (about) 94%, at least (about) 95%, at least (about) 96%, at least (about) 97%, at least (about) 98%, at least (about) 99%, or 100% sequence identity to an amino acid sequence of a glucose regulating peptide or a glucagon-like peptide (native or synthetic analog) set forth in Tables 3a-3c (such as one described more fully hereinbelow in the GLUCOSE REGULATING PEPTIDES section), or to an amino acid sequence of a protein relating to metabolic disorders and cardiology set forth in Table 3d (such as one described more fully hereinbelow in the METABOLIC DISEASE AND CARDIOVASCULAR PROTEINS section), or to an amino acid sequence of a growth hormone set forth in Table 3f (such as one described more fully hereinbelow in the GROWTH HORMONE PROTEINS section), or to an amino acid sequence of a cytokine set forth in Table 3g (such as one described more fully hereinbelow in the CYTOKINES section), or to an amino acid sequence of a transduction domain in Table 3h (such as one described more fully hereinbelow). In some embodiments of the compositions of this disclosure, the sequence of the BP can comprise one or more substitutions shown in Table 4 (such as one described more fully hereinbelow).

In some embodiments of the compositions disclosed herein, where the biologically active moiety is a biologically active peptide (BP), the BP can comprise an antibody (e.g., a monospecific, bispecific, trispecific, or multispecific antibody) (as defined hereinabove, the term “antibody” includes, among other things, an antibody fragment) (such as one described more fully hereinbelow in the ANTIBODIES section). The antibody can comprise a binding domain (or binding moiety) having binding affinity for an effector cell antigen. The effector cell antigen can be expressed on the surface of an effector cell selected from a plasma cell, a T cell, a B cell, a cytokine induced killer cell (CIK cell), a mast cell, a dendritic cell, a regulatory T cell (RegT cell), a helper T cell, a myeloid cell, and a NK cell. The effector cell antigen can be expressed on or within an effector cell. The effector cell antigen can be expressed on a T cell, such as a CD4+, CD8+, or natural killer (NK) cell. The effector cell antigen can be expressed on the surface of a T cell. The effector cell antigen can be expressed on a B cell, master cell, dendritic cell, or myeloid cell. The binding domain (or binding moiety) can comprise VH and VL regions derived from a monoclonal antibody capable of binding human CD3. In some embodiments, where the binding domain (or binding moiety) having binding affinity for CD3, the binding domain (or binding moiety) can have binding affinity for a member of the CD3 complex, which includes in individual form or independently combined form all known CD3 subunits of the CD3 complex; for example, CD3 epsilon, CD3 delta, CD3 gamma, CD3 zeta, CD3 alpha and CD3 beta. The binding domain (or binding moiety) having binding affinity for CD3 can have binding affinity for CD3 epsilon, CD3 delta, CD3 gamma, CD3 zeta, CD3 alpha or CD3 beta. In some embodiments of the compositions of this disclosure, the binding domain (or binding moiety) binding human CD3 can be derived from an anti-CD3 antibody selected from the group of antibodies set forth in Tables 5a-5e. The binding domain (or binding moiety) binding human CD3 can comprise VH and VL regions, where each VH and VL regions exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99%, or 100% sequence identity to paired VL and VH sequences of an anti-CD3 antibody selected from those set forth in Table 5a or Table 5d. The binding domain (or binding moiety) binding human CD3 can comprise VH and VL regions, where each VH and VL regions exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99%, or 100% sequence identity to paired VL and VH sequences of the huUCHT1 anti-CD3 antibody of Table 5a. The binding domain (or binding moiety) binding human CD3 can comprise a CDR-H1 region, a CDR-H2 region, a CDR-H3 region, a CDR-L1 region, a CDR-L2 region, and a CDR-H3 region, wherein each of the regions can be derived from a monoclonal antibody selected from the group of antibodies set forth in Tables 5a-5b or Table 5d. The binding domain (or binding moiety) binding human CD3 can comprise FRs each independently exhibiting at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99%, or 100% sequence identity to a corresponding FR set forth in Table 5c. The binding domain (or binding moiety) binding human CD3 can comprise a single-chain variable fragment (scFv) sequence exhibiting at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99%, or 100% sequence identity to an anti-CD3 scFv sequence set forth in Table 5e. In the foregoing embodiments, the VH and/or VL domains can be configured as scFv, diabodies, a single domain antibody, or a single domain camelid antibody. The antibody can comprise a binding domain (or binding moiety) having specific binding affinity to a tumor-specific marker or an antigen of a target cell (or a target antigen). The tumor-specific marker or the antigen of the target cell can be selected from the group consisting of alpha 4 integrin, Ang2, B7-H3, B7-H6 (e.g., its natural ligand Nkp30 rather than an antibody fragment), CEACAM5, cMET, CTLA4, FOLR1, EpCAM (epithelial cell adhesion molecule), CCR5, CD19, HER2, HER2 neu, HER3, HER4, HER1 (EGFR), PD-L1, PSMA, CEA, TROP-2, MUC1(mucin), MUC-2, MUC3, MUC4, MUC5AC, MUC5B, MUC7, MUC16, βhCG, Lewis-Y, CD20, CD33, CD38, CD30, CD56 (NCAM), CD133, ganglioside GD3, 9-O-acetyl-GD3, GM2, Globo H, fucosyl GM1, GD2, carbonicanhydrase IX, CD44v6, Nectin-4, Sonic Hedgehog (Shh), Wue-1, plasma cell antigen 1 (PC-1), melanoma chondroitin sulfate proteoglycan (MCSP), CCR8, 6-transmembrane epithelial antigen of prostate (STEAP), mesothelin, A33 antigen, prostate stem cell antigen (PSCA), Ly-6, desmoglein 4, fetal acetylcholine receptor (fnAChR), CD25, cancer antigen 19-9 (CA19-9), cancer antigen 125 (CA-125), Muellerian inhibitory substance receptor type II (MISIIR), sialylated Tn antigen (sTN), fibroblast activation antigen (FAP), endosialin (CD248), epidermal growth factor receptor variant III (EGFRvIII), tumor-associated antigen L6 (TAL6), SAS, CD63, TAG72, Thomsen-Friedenreich antigen (TF-antigen), insulin-like growth factor I receptor (IGF-IR), Cora antigen, CD7, CD22, CD70 (e.g., its natural ligand, CD27 rather than an antibody fragment), CD79a, CD79b, G250, MT-MMPs, fibroblast activation antigen (FAP), alpha-fetoprotein (AFP), VEGFR1, VEGFR2, DLK1, SP17, ROR1, EphA2, ENPP3, glypican 3 (GPC3), and TPBG/5T4 (trophoblast glycoprotein). The tumor-specific marker or the antigen of the target cell can be selected from alpha 4 integrin, Ang2, CEACAM5, cMET, CTLA4, FOLR1, EpCAM (epithelial cell adhesion molecule), CD19, HER2, HER2 neu, HER3, HER4, HER1 (EGFR), PD-L1, PSMA, CEA, TROP-2, MUC1(mucin), Lewis-Y, CD20, CD33, CD38, mesothelin, CD70 (e.g., its natural ligand, CD27 rather than an antibody fragment), VEGFR1, VEGFR2, ROR1, EphA2, ENPP3, glypican 3 (GPC3), and TPBG/5T4 (trophoblast glycoprotein). The tumor-specific marker or the antigen of the target cell can be any one set forth in the “Target” column of Table 6. The binding domain (or binding moiety) with binding affinity to the tumor-specific marker or the target cell antigen can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99%, or 100%, sequence identity to any one of the paired VL and VH sequences set forth in the “VH Sequences” and “VL Sequences” columns of Table 6. Without limiting the scope, additional exemplary tumor antigen target(s) can be selected from the group consisting of: FGFR2, LIV1, TRK, RET, BCMA, CD71, CD166, SSTR2, cKIT, VISTA, GPNMB, DLL3, CD123, LAMP1, P-Cadherin, Ephrin-A4, PTK7, NaPi2b, GCC, C4.4a, Mucin 17, FLT3, NKG2D ligands, SLAMF7, IL13a2R, CLL-1/CLEC12A, CD66e, IL3Ra, CD5, ULBP1, B7H4, CSPG4, SDC1, IL1RAP, Survivin, CD138, CD74, TIM1, SLITRK6, CD37, CD142, AXL, ETBR, Cadherin 6, FGFR3, CA6, CanAg (novel glycophorm of Muc 1), Integrin alpha V, Cripto 1 (TDGF1), CD352, and NOTCH3.

The bioactivity of the BP embodiments described herein can be evaluated by using assays or measured/determined parameters as described herein, and those sequences that retain at least (about) 40%, or at least (about) 50%, or at least (about) 55%, or at least (about) 60%, or at least (about) 70%, or at least (about) 80%, or at least (about) 90%, or at least (about) 95% or more activity compared to the corresponding native BP sequence would be considered suitable for inclusion in the compositions of this disclosure.

### Glucose Regulating Peptides

Endocrine and obesity-related diseases or disorders have reached epidemic proportions in most developed nations, and represent a substantial and increasing health care burden in most developed nations, which include a large variety of conditions affecting the organs, tissues, and circulatory system of the body. Of particular concern are endocrine and obesity-related diseases and disorders, which. Chief amongst these is diabetes; one of the leading causes of death in the United States. Diabetes is divided into two major sub-classes-Type I, also known as juvenile diabetes, or Insulin-Dependent Diabetes Mellitus (IDDM), and Type II, also known as adult onset diabetes, or Non-Insulin-Dependent Diabetes Mellitus (NIDDM). Type I Diabetes is a form of autoimmune disease that completely or partially destroys the insulin producing cells of the pancreas in such subjects, and requires use of exogenous insulin during their lifetime. Even in well-managed subjects, episodic complications can occur, some of which are life-threatening.

In Type II diabetics, rising blood glucose levels after meals do not properly stimulate insulin production by the pancreas. Additionally, peripheral tissues are generally resistant to the effects of insulin, and such subjects often have higher than normal plasma insulin levels (hyperinsulinemia) as the body attempts to overcome its insulin resistance. In advanced disease states insulin secretion is also impaired.

Insulin resistance and hyperinsulinemia have also been linked with two other metabolic disorders that pose considerable health risks: impaired glucose tolerance and metabolic obesity. Impaired glucose tolerance is characterized by normal glucose levels before eating, with a tendency toward elevated levels (hyperglycemia) following a meal. These individuals are considered to be at higher risk for diabetes and coronary artery disease. Obesity is also a risk factor for the group of conditions called insulin resistance syndrome, or “Syndrome X,” as is hypertension, coronary artery disease (arteriosclerosis), and lactic acidosis, as well as related disease states. The pathogenesis of obesity is believed to be multifactorial but an underlying problem is that in the obese, nutrient availability and energy expenditure are not in balance until there is excess adipose tissue. Other related diseases or disorders include, but are not limited to, gestational diabetes, juvenile diabetes, obesity, excessive appetite, insufficient satiety, metabolic disorder, glucagonomas, retinal neurodegenerative processes, and the “honeymoon period” of Type I diabetes.

Dyslipidemia is a frequent occurrence among diabetics; typically characterized by elevated plasma triglycerides, low HDL (high density lipoprotein) cholesterol, normal to elevated levels of LDL (low density lipoprotein) cholesterol and increased levels of small dense, LDL particles in the blood. Dyslipidemia is a main contributor to an increased incidence of coronary events and deaths among diabetic subjects.

Most metabolic processes in glucose homeostatis and insulin response are regulated by multiple peptides and hormones, and many such peptides and hormones, as well as analogues thereof, have found utility in the treatment of metabolic diseases and disorders. Many of these peptides tend to be highly homologous to each other, even when they possess opposite biological functions. Glucose-increasing peptides are exemplified by the peptide hormone glucagon, while glucose-lowering peptides include exendin-4, glucagon-like peptide 1, and amylin. However, the use of therapeutic peptides and/or hormones, even when augmented by the use of small molecule drugs, has met with limited success in the management of such diseases and disorders. In particular, dose optimization is important for drugs and biologics used in the treatment of metabolic diseases, especially those with a narrow therapeutic window. Hormones in general, and peptides involved in glucose homeostasis often have a narrow therapeutic window. The narrow therapeutic window, coupled with the fact that such hormones and peptides typically have a short half-life, which necessitates frequent dosing in order to achieve clinical benefit, results in difficulties in the management of such patients. While chemical modifications to a therapeutic protein, such as pegylation, can modify its in vivo clearance rate and subsequent serum half-life, it requires additional manufacturing steps and results in a heterogeneous final product. In addition, unacceptable side effects from chronic administration have been reported. Alternatively, genetic modification by fusion of an Fc domain to the therapeutic protein or peptide increases the size of the therapeutic protein, reducing the rate of clearance through the kidney, and promotes recycling from lysosomes by the FcRn receptor. Unfortunately, the Fc domain does not fold efficiently during recombinant expression and tends to form insoluble precipitates known as inclusion bodies. These inclusion bodies must be solubilized and functional protein must be renatured; a time-consuming, inefficient, and expensive process.

In some embodiments of the compositions of this disclosure, the biologically active peptide (BP) can comprise peptides involved in glucose homoestasis, insulin resistance and obesity (collectively, “glucose regulating peptides”), which compositions have utility in the treatment of glucose, insulin, and obesity disorders, disease and related conditions. Glucose regulating peptides can include any protein of biologic, therapeutic, or prophylactic interest or function that is useful for preventing, treating, mediating, or ameliorating a disease, disorder or condition of glucose homeostasis or insulin resistance or obesity. Suitable glucose-regulating peptides that can be linked to a masking moiety (such as XTEN) can include all biologically active polypeptides that increase glucose-dependent secretion of insulin by pancreatic beta-cells or potentiate the action of insulin. Glucose-regulating peptides can also include all biologically active polypeptides that stimulate pro-insulin gene transcription in the pancreatic beta-cells. Furthermore, glucose-regulating peptides can also include all biologically active polypeptides that slow down gastric emptying time and reduce food intake. Glucose-regulating peptides can also include all biologically active polypeptides that inhibit glucagon release from the alpha cells of the Islets of Langerhans. Table 3a provides a non-limiting list of sequences of glucose regulating peptides that can be encompassed by the compositions of this disclosure. In some embodiments of the compositions disclosed herein, where the biologically active moiety can be a biologically active peptide (BP), the BP can comprise a peptide sequence that exhibits at least (about) 80% sequence identity (e.g., at least (about) 81%, at least (about) 82%, at least (about) 83%, at least (about) 84%, at least (about) 85%, at least (about) 86%, at least (about) 87%, at least (about) 88%, at least (about) 89%, at least (about) 90%, at least (about) 91%, at least (about) 92%, at least (about) 93%, at least (about) 94%, at least (about) 95%, at least (about) 96%, at least (about) 97%, at least (about) 98%, at least (about) 99%, or 100% sequence identity) to an amino acid sequence of a glucose regulating peptide set forth in Table 3a.

TABLE 3a
Glucose-Regulating Peptides

| Name of Protein | SEQ ID | |
|---|---|---|
| (Synonym) | NO: | Amino Acid Sequence |
| Adrenomedullin (ADM) | 233 | YRQSMNNFQGLRSFGCRFGTCTVQKLAHQIYQFTDKDKD |
| | | NVAPRSKISPQGY |
| Amylin, rat | 234 | KCNTATCATQRLANFLVRSSNNLGPVLPPTNVGSNTY |
| Amylin, human | 235 | KCNTATCATQRLANFLVHSSNNFGAILSSTNVGSNTY |
| Calcitonin (hCT) | 236 | CGNLSTCMLGTYTQDFNKFHTFPQTAIGVGAP |
| Calcitonin, salmon | 237 | CSNLSTCVLGKLSQELHKLQTYPRTNTGSGTP |
| Calcitonin gene related peptide | 238 | ACDTATCVTHRLAGLLSRSGGVVKNMVPTNVGSKAF |
| (h-CGRP α) | | |
| Calcitonin gene related peptide | 239 | ACNTATCVTHRLAGLLSRSGGMVKSNFVPTNVGSKAF |
| (h-CGRP β) | | |
| cholecystokinin (CCK) | 240 | MNSGVCLCVLMAVLAAGALTQPVPPADPAGSGLQRAEE |
| | | APRRQLRVSQRTDGESRAHLGALLARYIQQARKAPSGRM |
| | | SIVKNLQNLDPSHRISDRDYMGWMDFGRRSAEEYEYPS |
| CCK-33 | 241 | KAPSGRMSIVKNLQNLDPSHRISDRDYMGWMDF |
| CCK-8 | 242 | DYMGWMDF |
| Exendin-3 | 243 | HSDGTFTSDLSKQMEEEAVRLFIEWLKNGGPSSGAPPPS |
| Exendin-4 | 244 | HGEGTFTSDLSKQMEEEAVR LFIEWLKNGGPSSGAPPPS |
| FGF-19 | 245 | MRSGCVVVHVWILAGLWLAVAGRPLAFSDAGPHVHYG |
| | | WGDPIRLRHLYTSGPHGLSSCFLRIRADGVVDCARGQSAH |
| | | SLLEIKAVALRTVAIKGVHSVRYLCMGADGKMQGLLQYS |
| | | EEDCAFEEEIRPDGYNVYRSEKHRLPVSLSSAKQRQLYKN |
| | | RGFLPLSHFLPMLPMVPEEPEDLRGHLESDMFSSPLETDSM |
| | | DPFGLVTGLEAVRSPSFEK |
| FGF-21 | 246 | MDSDETGFEHSGLWVSVLAGLLLGACQAHPIPDSSPLLQF |
| | | GGQVRQRYLYTDDAQQTEAHLEIREDGTVGGAADQSPES |
| | | LLQLKALKPGVIQILGVKTSRFLCQRPDGALYGSLHFDPEA |
| | | CSFRELLLEDGYNVYQSEAHGLPLHLPGNKSPHRDPAPRG |
| | | PARFLPLPGLPPALPEPPGILAPQPPDVGSSDPLSMVGPSQG |
| | | RSPSYAS |
| Gastrin | 247 | QLGPQGPPHLVADPSKKQGPWLEEEEEAYGWMDF |
| Gastrin-17 | 248 | DPSKKQGPWLEEEEEAYGWMDF |
| Gastric inhibitory polypeptide | 249 | YAEGTFISDYSIAMDKIHQQDFVNWLLAQKGKKNDWKH |
| (GIP) | | NITQ |
| Ghrelin | 250 | GSSFLSPEHQRVQQRKESKKPPAKLQPR |
| Glucagon | 251 | HSQGTFTSDYSKYLDSRRAQDFVQWLMNT |
| Glucagon-like peptide-1 | 252 | HDEFERHAEGTFTSDVSSTLEGQAALEFIAWLVKGRG |
| (hGLP-1) (GLP-1; 1-37) | | |
| GLP-1 (7-36), human | 253 | HAEGTFTSDVSSYLEGQAALEFIAWLVKGR |
| GLP-1 (7-37), human | 254 | HAEGTFTSDVSSTLEGQAALEFIAWLVKGRG |
| GLP-1, frog | 255 | HAEGTYTNDVTEYLEEKAAKEFIEWLIKGKPKKIRYS |
| Glucagon-like peptide 2 (GLP- | 256 | HADGSFSDEMNTILDNLAARDFINWLIETKITD |
| 2), human | | |
| GLP-2, frog | 257 | HAEGTFTNDMTNYLEEKAAKEFVGWLIKGRP-OH |
| IGF-1 | 258 | GPETLCGAELVDALQFVCGDRGFYFNKPTGYGSSSRRAPQ |
| | | TGIVDECCFRSCDLRRLEMYCAPLKPAKSA |
| IGF-2 | 259 | AYRPSETLCGGELVDTLQFVCGDRGFYFSRPASRVSRRSR |
| | | GIVEECCFRSCDLALLETYCATPAKSE |
| INGAP peptide | 260 | EESQKKLPSSRITCPQGSVAYGSYCYSLILIPQTWSNAELSC |
| (islet neogenesis-associated | | QMHFSGHLAFLLSTGEITFVSSLVKNSLTAYQYIWIGLHDP |
| protein) | | SHGTLPNGSGWKWSSSNVLTFYNWERNPSIAADRGYCAV |
| | | LSQKSGFQKWRDFNCENELPYICKFKV |
| Intermedin (AFP-6) | 261 | TQAQLLRVGCVLGTCQVQNLSHRLWQLMGPAGRQDSAP |
| | | VDPSSPHSY |
| Leptin, human | 262 | VPIQKVQDDTKTLIKTIVTRINDISHTQSVSSKQKVTGLDFI |
| | | PGLHPILTLSKMDQTLAVYQQILTSMPSRNVIQISNDLENL |
| | | RDLLHVLAFSKSCHLPWASGLETLDSLGGVLEASGYSTEV |
| | | VALSRLQGSLQDMLWQLDLSPGC |
| Neuromedin (U-8) porcine | 263 | YFLFRPRN |
| Neuromedin (U-9) | 264 | GYFLFRPRN |
| neuromedin (U25) human) | 265 | FRVDEEFQSPFASQSRGYFLFRPRN |
| Neuromedin (U25) pig | 266 | FKVDEEFQGPIVSQNRRYFLFRPRN |
| Neuromedin S, human | 267 | ILQRGSGTAAVDFTKKDHTATWGRPFFLFRPRN |
| Neuromedin U, rat | 268 | YKVNEYQGPVAPSGGFFLFRPRN |
| oxyntomodulin (OXM) | 269 | HSQGTFTSDYSKYLDSRRAQDFVQWLMNTKRNRNNIA |
| Peptide YY (PYY) | 270 | YPIKPEAPGEDASPEELNRYYASLRHYLNLVTRQRY |
| Pramlintide | 271 | KCNTATCATNRLANFLVHSSNNFGPILPPTNVGSNTY-NH2 |
| Urocortin (Ucn-1) | 272 | DNPSLSIDLTFHLLRTLLELARTQSQRERAEQNRIIFDSV |
| Urocortin (Ucn-2) | 273 | IVLSLDVPIGLLQILLEQARARAAREQATTNARILARVGHC |
| Urocortin (Ucn-3) | 274 | FTLSLDVPTNIMNLLFNIAKAKNLRAQAAANAHLMAQI |

“Adrenomedullin” or “ADM” means the human adrenomedulin peptide hormone and species and sequence variants thereof having at least a portion of the biological activity of mature ADM. ADM is generated from a 185 amino acid preprohormone through consecutive enzymatic cleavage and amidation, resulting in a 52 amino acid bioactive peptide with a measured plasma half-life of 22 min. ADM-containing fusion proteins of the invention may find particular use in diabetes for stimulatory effects on insulin secretion from islet cells for glucose regulation or in subjects with sustained hypotension. The complete genomic infrastructure for human AM has been reported (Ishimitsu, et al., Biochem. Biophys. Res. Commun 203:631-639 (1994)), and analogs of ADM peptides have been cloned, as described in U.S. Pat. No. 6,320,022.

“Amylin” means the human peptide hormone referred to as amylin, pramlintide, and species variations thereof, as described in U.S. Pat. No. 5,234,906, having at least a portion of the biological activity of mature amylin. Amylin is a 37-amino acid polypeptide hormone co-secreted with insulin by pancreatic beta cells in response to nutrient intake (Koda et al., Lancet 339:1179-1180. 1992), and has been reported to modulate several key pathways of carbohydrate metabolism, including incorporation of glucose into glycogen. Amylin-containing fusion proteins of the invention may find particular use in diabetes and obesity for regulating gastric emptying, suppressing glucagon secretion and food intake, thereby affecting the rate of glucose appearance in the circulation. Thus, the fusion proteins may complement the action of insulin, which regulates the rate of glucose disappearance from the circulation and its uptake by peripheral tissues. Amylin analogues have been cloned, as described in U.S. Pat. Nos. 5,686,411 and 7,271,238. Amylin mimetics can be created that retain biologic activity. For example, pramlintide has the sequence KCNTATCATNRLANFLVHSSNNFGPILPPTNVGSNTY (SEQ ID NO: 271), wherein amino acids from the rat amylin sequence are substituted for amino acids in the human amylin sequence. In one embodiment, the invention contemplates fusion proteins comprising amylin mimetics of the sequence KCNTATCATX₁RLANFLVHSSNNFGX₂ILX₂X₂TNVGSNTY (SEQ ID NO: 275), wherein X₁ is independently N or Q and X₂ is independently S, P or G. In one embodiment, the amylin mimetic incorporated into a composition of this disclosure can have the sequence KCNTATCATNRLANFLVHSSNNFGGILGGTNVGSNTY (SEQ ID NO: 276). In another embodiment, wherein the amylin mimetic is used at the C-terminus of the composition, the mimetic can have the sequence KCNTATCATNRLANFLVHSSNNFGGILGGTNVGSNTY(NH₂) (SEQ ID NO: 276).

“Calcitonin” (CT) means the human calcitonin protein and species and sequence variants thereof, including salmon calcitonin (“sCT”), having at least a portion of the biological activity of mature CT. CT is a 32 amino acid peptide cleaved from a larger prohormone of the thyroid that appears to function in the nervous and vascular systems, but has also been reported to be a potent hormonal mediator of the satiety reflex. CT is named for its secretion in response to induced hypercalcemia and its rapid hypocalcemic effect. It is produced in and secreted from neuroendocrine cells in the thyroid termed C cells. CT has effects on the osteoclast, and the inhibition of osteoclast functions by CT results in a decrease in bone resorption. In vitro effects of CT include the rapid loss of ruffled borders and decreased release of lysosomal enzymes. A major function of CT(1-32) is to combat acute hypercalcemia in emergency situations and/or protect the skeleton during periods of “calcium stress” such as growth, pregnancy, and lactation. (Reviewed in Becker, JCEM, 89(4): 1512-1525 (2004) and Sexton, Current Medicinal Chemistry 6: 1067-1093 (1999)). Calcitonin-containing fusion proteins of the invention may find particular use for the treatment of osteoporosis and as a therapy for Paget's disease of bone. Synthetic calcitonin peptides have been created, as described in U.S. Pat. Nos. 5,175,146 and 5,364,840.

“Calcitonin gene related peptide” or “CGRP” means the human CGRP peptide and species and sequence variants thereof having at least a portion of the biological activity of mature CGRP. Calcitonin gene related peptide is a member of the calcitonin family of peptides, which in humans exists in two forms, α-CGRP (a 37 amino acid peptide) and β-CGRP. CGRP has 43-46% sequence identity with human amylin. CGRP-containing fusion proteins of the invention may find particular use in decreasing morbidity associated with diabetes, ameliorating hyperglycemia and insulin deficiency, inhibition of lymphocyte infiltration into the islets, and protection of beta cells against autoimmune destruction. Methods for making synthetic and recombinant CGRP are described in U.S. Pat. No. 5,374,618.

“Cholecystokinin” or “CCK” means the human CCK peptide and species and sequence variants thereof having at least a portion of the biological activity of mature CCK. CCK-58 is the mature sequence, while the CCK-33 amino acid sequence first identified in humans is the major circulating form of the peptide. The CCK family also includes an 8-amino acid in vivo C-terminal fragment (“CCK-8”), pentagastrin or CCK-5 being the C-terminal peptide CCK(29-33), and CCK-4 being the C-terminal tetrapeptide CCK(30-33). CCK is a peptide hormone of the gastrointestinal system responsible for stimulating the digestion of fat and protein. CCK-33 and CCK-8-containing fusion proteins of the invention may find particular use in reducing the increase in circulating glucose after meal ingestion and potentiating the increase in circulating insulin. Analogues of CCK-8 have been prepared, as described in U.S. Pat. No. 5,631,230. 1002171″Exendin-3″ means a glucose regulating peptide isolated from Heloderma horridum and sequence variants thereof having at least a portion of the biological activity of mature exendin-3. Exendin-3 amide is a specific exendin receptor antagonist from that mediates an increase in pancreatic cAMP, and release of insulin and amylase. Exendin-3-containing fusion proteins of the invention may find particular use in the treatment of diabetes and insulin resistance disorders. The sequence and methods for its assay are described in U.S. Pat. No. 5,424,286.

Exendin-4″ means a glucose regulating peptide found in the saliva of the Gila-monster Heloderma suspectum, as well as species and sequence variants thereof, and includes the native 39 amino acid sequence His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser and homologous sequences and peptide mimetics, and variants thereof; natural sequences, such as from primates and non-natural having at least a portion of the biological activity of mature exendin-4. Exendin-4 is an incretin polypeptide hormone that decreases blood glucose, promotes insulin secretion, slows gastric emptying and improves satiety, providing a marked improvement in postprandial hyperglycemia. The exendins have some sequence similarity to members of the glucagon-like peptide family, with the highest identity being to GLP-1 (Goke, et al., J. Biol. Chem., 268:19650-55 (1993)). A variety of homologous sequences can be functionally equivalent to native exendin-4 and GLP-1. Conservation of GLP-1 sequences from different species are presented in Regulatory Peptides 2001 98 p. 1-12. Table 3b shows the sequences from a wide variety of species, while Table 3c shows a list of synthetic GLP-1 analogs; all of which are contemplated for use in the composition described herein. Exendin-4 binds at GLP-1 receptors on insulin-secreting OTC′ cells, and also stimulates somatostatin release and inhibits gastrin release in isolated stomachs (Goke, et al., J. Biol. Chem. 268:19650-55, 1993). As a mimetic of GLP-1, exendin-4 displays a similar broad range of biological activities, yet has a longer half-life than GLP-1, with a mean terminal half-life of 2.4 h. Exenatide is a synthetic version of exendin-4, marketed as Byetta. However, due to its short half-life, exenatide is currently dosed twice daily, limiting its utility. Exendin-4-containing fusion proteins of the invention may find particular use in the treatment of diabetes and insulin resistance disorders.

‘Fibroblast growth factor 21’, or “FGF-21” means the human protein encoded by the FGF21 gene, or species and sequence variants thereof having at least a portion of the biological activity of mature FGF21. FGF-21 stimulates glucose uptake in adipocytes but not in other cell types; the effect is additive to the activity of insulin. FGF-21 injection in ob/ob mice results in an increase in Glut1 in adipose tissue. FGF21 also protects animals from diet-induced obesity when over expressed in transgenic mice and lowers blood glucose and triglyceride levels when administered to diabetic rodents (Kharitonenkov A, et al., (2005). “FGF-21 as a novel metabolic regulator”. J. Clin. Invest. 115: 1627-35). FGF-21-containing fusion proteins of the invention may find particular use in treatment of diabetes, including causing increased energy expenditure, fat utilization and lipid excretion. FGF-21 has been cloned, as disclosed in U.S. Pat. No. 6,716,626.

“FGF-19”, or “fibroblast growth factor 19” means the human protein encoded by the FGF19 gene, or species and sequence variants thereof having at least a portion of the biological activity of mature FGF-19. FGF-19 is a protein member of the fibroblast growth factor (FGF) family. FGF family members possess broad mitogenic and cell survival activities, and are involved in a variety of biological processes. FGF-19 increases liver expression of the leptin receptor, metabolic rate, stimulates glucose uptake in adipocytes, and leads to loss of weight in an obese mouse model (Fu, L, et al. FGF-19-containing fusion proteins of the invention may find particular use in increasing metabolic rate and reversal of dietary and leptin-deficient diabetes. FGF-19 has been cloned and expressed, as described in US Patent Application No. 20020042367.

“Gastrin” means the human gastrin peptide, truncated versions, and species and sequence variants thereof having at least a portion of the biological activity of mature gastrin. Gastrin is a linear peptide hormone produced by G cells of the duodenum and in the pyloric antrum of the stomach and is secreted into the bloodstream. Gastrin is found primarily in three forms: gastrin-34 (“big gastrin”); gastrin-17 (“little gastrin”); and gastrin-14 (“minigastrin”). It shares sequence homology with CCK. Gastrin-containing fusion proteins of the invention may find particular use in the treatment of obesity and diabetes for glucose regulation. Gastrin has been synthesized, as described in U.S. Pat. No. 5,843,446.

“Ghrelin” means the human hormone that induces satiation, or species and sequence variants thereof, including the native, processed 27 or 28 amino acid sequence and homologous sequences. Ghrelin is produced mainly by P/D1 cells lining the fundus of the human stomach and epsilon cells of the pancreas that stimulates hunger, and is considered the counterpart hormone to leptin. Ghrelin levels increase before meals and decrease after meals, and can result in increased food intake and increase fat mass by an action exerted at the level of the hypothalamus. Ghrelin also stimulates the release of growth hormone. Ghrelin is acylated at a serine residue by n-octanoic acid; this acylation is essential for binding to the GHS1a receptor and for the GH-releasing capacity of ghrelin. Ghrelin-containing fusion proteins of the invention may find particular use as agonists; e.g., to selectively stimulate motility of the GI tract in gastrointestinal motility disorder, to accelerate gastric emptying, or to stimulate the release of growth hormone. Ghrelin analogs with sequence substitutions or truncated variants, such as described in U.S. Pat. No. 7,385,026, may find particular use as fusion partners to XTEN for use as antagonists for improved glucose homeostasis, treatment of insulin resistance and treatment of obesity. The isolation and characterization of ghrelin has been reported (Kojima M, et al., Ghrelin is a growth-hormone-releasing acylated peptide from stomach. Nature. 1999; 402(6762):656-660.) and synthetic analogs have been prepared by peptide synthesis, as described in U.S. Pat. No. 6,967,237.

“Glucagon” means the human glucagon glucose regulating peptide, or species and sequence variants thereof, including the native 29 amino acid sequence and homologous sequences; natural, such as from primates, and non-natural sequence variants having at least a portion of the biological activity of mature glucagon. The term “glucagon” as used herein also includes peptide mimetics of glucagon. Native glucagon is produced by the pancreas, released when blood glucose levels start to fall too low, causing the liver to convert stored glycogen into glucose and release it into the bloodstream. While the action of glucagon is opposite that of insulin, which signals the body's cells to take in glucose from the blood, glucagon also stimulates the release of insulin, so that newly-available glucose in the bloodstream can be taken up and used by insulin-dependent tissues. Glucagon-containing fusion proteins of the invention may find particular use in increasing blood glucose levels in individuals with extant hepatic glycogen stores and maintaining glucose homeostasis in diabetes. Glucagon has been cloned, as disclosed in U.S. Pat. No. 4,826,763.

“GLP-1” means human glucagon like peptide-1 and sequence variants thereof having at least a portion of the biological activity of mature GLP-1. The term “GLP-1” includes human GLP-1(1-37), GLP-1(7-37), and GLP-1(7-36)amide. GLP-1 stimulates insulin secretion, but only during periods of hyperglycemia. The safety of GLP-1 compared to insulin is enhanced by this property and by the observation that the amount of insulin secreted is proportional to the magnitude of the hyperglycemia. The biological half-life of GLP-1(7-37)OH is a mere 3 to 5 minutes (U.S. Pat. No. 5,118,666). GLP-1-containing fusion proteins of the invention may find particular use in the treatment of diabetes and insulin-resistance disorders for glucose regulation. GLP-1 has been cloned and derivatives prepared, as described in U.S. Pat. No. 5,118,666. Non-limited examples of glucagon-like peptide sequences from a wide variety of species, and synthetic analogs thereof, are shown in Tables 3b-3c. In some embodiments of the compositions disclosed herein, where the biologically active moiety can be a biologically active peptide (BP), the BP can comprise a peptide sequence that exhibits at least (about) 80% sequence identity (e.g., at least (about) 81%, at least (about) 82%, at least (about) 83%, at least (about) 84%, at least (about) 85%, at least (about) 86%, at least (about) 87%, at least (about) 88%, at least (about) 89%, at least (about) 90%, at least (about) 91%, at least (about) 92%, at least (about) 93%, at least (about) 94%, at least (about) 95%, at least (about) 96%, at least (about) 97%, at least (about) 98%, at least (about) 99%, or 100% sequence identity) to an amino acid sequence of a glucagon-like peptide (native or synthetic analog) set forth in Tables 3b-3c.

TABLE 3b
Representative Naturally-Occurring GLP-1 Homologs as BP Candidates

| | SEQ ID | |
|---|---|---|
| Gene Name | NO: | Amino Acid Sequence |
| GLP-1 [frog] | 277 | HAEGTYTNDVTEYLEEKAAKEFIEWLIKGKPKKIRYS |
| GLP-la [Xenopus laevis] | 278 | HAEGTFTSDVTQQLDEKAAKEFIDWLINGGPSKEIIS |
| GLP-16 [Xenopus laevis] | 279 | HAEGTYTNDVTEYLEEKAAKEFIIEWLIKGKPK |
| GLP-1c [Xenopus laevis] | 280 | HAEGTFTNDMTNYLEEKAAKEFVGWLIKGRPK |
| Gastric Inhibitory | 281 | HAEGTFISDYSIAMDKIRQQDFVNWLL |
| Polypeptide [Mus musculus] | | |
| Glucose-dependent | 282 | HAEGTFISDYSIAMDKIRQQDFVNWLL |
| insulinotropic polypeptide | | |
| [Equus caballus] | | |
| Glucagon-like peptide | 283 | HADGTFTNDMTSYLDAKAARDFVSWLARSDKS |
| [Petromyzon marinus] | | |
| Glucagon-like peptide | 284 | HAEGTYTSDVSSYLQDQAAKEFVSWLKTGR |
| [Anguilla rostrata] | | |
| Glucagon-like peptide | 285 | HAEGTYTSDVSSYLQDQAAKEFVSWLKTGR |
| [Anguilla anguilla] | | |
| Glucagon-like peptide | 286 | HADGIYTSDVASLTDYLKSKRFVESLSNYNKRQNDRRM |
| [Hydrolagus colliei] | | |
| Glucagon-like peptide | 287 | YADAPYISDVYSYLQDQVAKKWLKSGQDRRE |
| [Amia calva] | | |
| GLUC_ICTPU/38-65 | 288 | HADGTYTSDVSSYLQEQAAKDFITWLKS |
| GLUCL_ANGRO/1-28 | 289 | HAEGTYTSDVSSYLQDQAAKEFVSWLKT |
| GLUC_BOVIN/98-125 | 290 | HAEGTFTSDVSSYLEGQAAKEFIAWLVK |
| GLUC1_LOPAM/91-118 | 291 | HADGTFTSDVSSYLKDQAIKDFVDRLKA |
| GLUCL_HYDCO/1-28 | 292 | HADGIYTSDVASLTDYLKSKRFVESLSN |
| GLUC_CAVPO/53-80 | 293 | HSQGTFTSDYSKYLDSRRAQQFLKWLLN |
| GLUC_CHIBR/1-28 | 294 | HSQGTFTSDYSKHLDSRYAQEFVQWLMN |
| GLUC1_LOPAM/53-80 | 295 | HSEGTFSNDYSKYLEDRKAQEFVRWLMN |
| GLUC_HYDCO/1-28 | 296 | HTDGIFSSDYSKYLDNRRTKDFVQWLLS |
| GLUC_CALMI/1-28 | 297 | HSEGTFSSDYSKYLDSRRAKDFVQWLMS |
| GIP_BOVIN/1-28 | 298 | YAEGTFISDYSIAMDKIRQQDFVNWLLA |
| VIP_MELGA/89-116 | 299 | HADGIFTTVYSHLLAKLAVKRYLHSLIR |
| PACA_CHICK/131-158 | 300 | HIDGIFTDSYSRYRKQMAVKKYLAAVLG |
| VIP_CAVPO/45-72 | 301 | HSDALFTDTYTRLRKQMAMKKYLNSVLN |
| VIP_DIDMA/1-28 | 302 | HSDAVFTDSYTRLLKQMAMRKYLDSILN |
| EXE1_HELSU/1-28 | 303 | HSDATFTAEYSKLLAKLALQKYLESILG |
| SLIB_CAPHI/1-28 | 304 | YADAIFTNSYRKVLGQLSARKLLQDIMN |
| SLIB_RAT/31-58 | 305 | HADAIFTSSYRRILGQLYARKLLHEIMN |
| SLIB_MOUSE/31-58 | 306 | HVDAIFTTNYRKLLSQLYARKVIQDIMN |
| PACA_HUMAN/83-110 | 307 | VAHGILNEAYRKVLDQLSAGKHLQSLVA |
| PACA_SHEEP/83-110 | 308 | VAHGILDKAYRKVLDQLSARRYLQTLMA |
| PACA_ONCNE/82-109 | 309 | HADGMFNKAYRKALGQLSARKYLHSLMA |
| GLUC_BOVIN/146-173 | 310 | HADGSFSDEMNTVLDSLATRDFINWLLQ |
| SECR_CANFA/1-27 | 311 | HSDGTFTSELSRLRESARLQRLLQGLV |
| SECR_CHICK/1-27 | 312 | HSDGLFTSEYSKMRGNAQVQKFIQNLM |
| EXE3_HELHO/48-75 | 313 | HSDGTFTSDLSKQMEEEAVRLFIEWLKN |

TABLE 3c
Representative GLP-1 Synthetic Analogs

| SEQ | |
|---|---|
| ID | |
| NO: | Amino Acid Sequence |
| 314 | HAEGTFTSDVSSYLEGQAAREFIAWLVKGRG |
| 315 | HAEGTFTSDVSSYLEGQAAKEFIAWLVRGRG |
| 316 | HAEGTFTSDVSSYLEGQAAKEFIAWLVKGKG |
| 317 | HAEGTFTSDVSSYLEGQAAREFIAWLVRGKG |
| 318 | HAEGTFTSDVSSYLEGQAAREFIAWLVRGKGR |
| 319 | HAEGTFTSDVSSYLEGQAAREFIAWLVRGRGRK |
| 320 | HAEGTFTSDVSSYLEGQAAREFIAWLVRGRGRRK |
| 321 | HAEGTFTSDVSSYLEGQAAREFIAWLVKGKG |
| 322 | HAEGTFTSDVSSYLEGQAAKEFIAWLVRGKG |
| 323 | HAEGTFTSDVSSYLEGQAAREFIAWLVKGRGRK |
| 324 | HAEGTFTSDVSSYLEGQAAKEFIAWLVRGRGRRK |
| 325 | HAEGTFTSDVSSYLEGQAAREFIAWLVRGKGRK |
| 326 | HAEGTFTSDVSSYLEGQAAREFIAWLVRGKGRRK |
| 327 | HGEGTFTSDVSSYLEGQAAREFIAWLVKGRG |
| 328 | HGEGTFTSDVSSYLEGQAAKEFIAWLVRGRG |
| 329 | HGEGTFTSDVSSYLEGQAAKEFIAWLVKGKG |
| 330 | HGEGTFTSDVSSYLEGQAAREFIAWLVRGKG |
| 331 | HGEGTFTSDVSSYLEGQAAREFIAWLVRGRGRK |
| 332 | HGEGTFTSDVSSYLEGQAAREFIAWLVRGRGRRK |
| 333 | HGEGTFTSDVSSYLEGQAAREFIAWLVKGKG |
| 334 | HGEGTFTSDVSSYLEGQAAKEFIAWLVRGKG |
| 335 | HGEGTFTSDVSSYLEGQAAREFIAWLVKGRGRK |
| 336 | HGEGTFTSDVSSYLEGQAAKEFIAWLVRGRGRRK |
| 337 | HGEGTFTSDVSSYLEGQAAREFIAWLVRGKGRK |
| 338 | HGEGTFTSDVSSYLEGQAAREFIAWLVRGKGRRK |
| 339 | HAEGTFTSDVSSYLEGQAAREFIAWLVRGRGK |
| 340 | HAEGTFTSDVSSYLEGQAAREFIAWLVRGRGRK |
| 341 | HAEGTFTSDVSSYLEGQAAREFIAWLVRGRGRRK |
| 342 | HAEGTFTSDVSSYLEGQAAREFIAWLVRGRGRREK |
| 343 | HAEGTFTSDVSSYLEGQAAREFIAWLVRGRGRREFK |
| 344 | HAEGTFTSDVSSYLEGQAAREFIAWLVRGRGRREFPK |
| 345 | HAEGTFTSDVSSYLEGQAAREFIAWLVRGRGRREFPEK |
| 346 | HAEGTFTSDVSSYLEGQAAREFIAWLVRGRGRREFPEEK |
| 347 | HDEFERHAEGTFTSDVSSYLEGQAAREFIAWLVRGRGK |
| 348 | HDEFERHAEGTFTSDVSSYLEGQAAREFIAWLVRGRGRK |
| 349 | HDEFERHAEGTFTSDVSSYLEGQAAREFIAWLVRGRGRRK |
| 350 | HDEFERHAEGTFTSDVSSYLEGQAAREFIAWLVRGRGRREK |
| 351 | HDEFERHAEGTFTSDVSSYLEGQAAREFIAWLVRGRGRREFK |
| 352 | HDEFERHAEGTFTSDVSSYLEGQAAREFIAWLVRGRGRREFPK |
| 353 | HDEFERHAEGTFTSDVSSYLEGQAAREFIAWLVRGRGRREFPEK |
| 354 | HDEFERHAEGTFTSDVSSYLEGQAAREFIAWLVRGRGRREFPEEK |
| 355 | DEFERHAEGTFTSDVSSYLEGQAAREFIAWLVRGRGRK |
| 356 | DEFERHAEGTFTSDVSSYLEGQAAREFIAWLVRGRGRRK |
| 357 | DEFERHAEGTFTSDVSSYLEGQAAREFIAWLVRGRGRREK |
| 358 | DEFERHAEGTFTSDVSSYLEGQAAREFIAWLVRGRGRREFK |
| 359 | DEFERHAEGTFTSDVSSYLEGQAAREFIAWLVRGRGRREFPK |
| 360 | DEFERHAEGTFTSDVSSYLEGQAAREFIAWLVRGRGRREFPEK |
| 361 | DEFERHAEGTFTSDVSSYLEGQAAREFIAWLVRGRGRREFPEEK |
| 362 | EFERHAEGTFTSDVSSYLEGQAAREFIAWLVRGRGK |
| 363 | EFERHAEGTFTSDVSSYLEGQAAREFIAWLVRGRGRK |
| 364 | EFERHAEGTFTSDVSSYLEGQAAREFIAWLVRGRGRRK |
| 365 | EFERHAEGTFTSDVSSYLEGQAAREFIAWLVRGRGRREK |
| 366 | EFERHAEGTFTSDVSSYLEGQAAREFIAWLVRGRGRREFK |
| 367 | EFERHAEGTFTSDVSSYLEGQAAREFIAWLVRGRGRREFPK |
| 368 | EFERHAEGTFTSDVSSYLEGQAAREFIAWLVRGRGRREFPEK |
| 369 | EFERHAEGTFTSDVSSYLEGQAAREFIAWLVRGRGRREFPEEK |
| 370 | FERHAEGTFTSDVSSYLEGQAAREFIAWLVRGRGK |
| 371 | FERHAEGTFTSDVSSYLEGQAAREFIAWLVRGRGRK |
| 372 | FERHAEGTFTSDVSSYLEGQAAREFIAWLVRGRGRRK |
| 373 | FERHAEGTFTSDVSSYLEGQAAREFIAWLVRGRGRREK |
| 374 | FERHAEGTFTSDVSSYLEGQAAREFIAWLVRGRGRREFK |
| 375 | FERHAEGTFTSDVSSYLEGQAAREFIAWLVRGRGRREFPK |
| 376 | FERHAEGTFTSDVSSYLEGQAAREFIAWLVRGRGRREFPEK |
| 377 | FERHAEGTFTSDVSSYLEGQAAREFIAWLVRGRGRREFPEEK |
| 378 | ERHAEGTFTSDVSSYLEGQAAREFIAWLVRGRGK |
| 379 | ERHAEGTFTSDVSSYLEGQAAREFIAWLVRGRGRK |
| 380 | ERHAEGTFTSDVSSYLEGQAAREFIAWLVRGRGRRK |
| 381 | ERHAEGTFTSDVSSYLEGQAAREFIAWLVRGRGRREK |
| 382 | ERHAEGTFTSDVSSYLEGQAAREFIAWLVRGRGRREFK |
| 383 | ERHAEGTFTSDVSSYLEGQAAREFIAWLVRGRGRREFPK |
| 384 | ERHAEGTFTSDVSSYLEGQAAREFIAWLVRGRGRREFPEK |
| 385 | ERHAEGTFTSDVSSYLEGQAAREFIAWLVRGRGRREFPEEK |
| 386 | RHAEGTFTSDVSSYLEGQAAREFIAWLVRGRGK |
| 387 | RHAEGTFTSDVSSYLEGQAAREFIAWLVRGRGRK |
| 388 | RHAEGTFTSDVSSYLEGQAAREFIAWLVRGRGRRK |
| 389 | RHAEGTFTSDVSSYLEGQAAREFIAWLVRGRGRREK |
| 390 | RHAEGTFTSDVSSYLEGQAAREFIAWLVRGRGRREFK |
| 391 | RHAEGTFTSDVSSYLEGQAAREFIAWLVRGRGRREFPK |
| 392 | RHAEGTFTSDVSSYLEGQAAREFIAWLVRGRGRREFPEK |
| 393 | RHAEGTFTSDVSSYLEGQAAREFIAWLVRGRGRREFPEEK |
| 394 | HDEFERHAEGTFTSDVSSYLEGQAAREFIAWLVKGRGK |
| 395 | HDEFERHAEGTFTSDVSSYLEGQAAKEFIAWLVRGRGK |
| 396 | HDEFERHAEGTFTSDVSSYLEGQAAREFIAWLVRGKGK |
| 397 | HAEGTFTSDVSSYLEGQAAREFIAWLVKGRGK |
| 398 | HAEGTFTSDVSSYLEGQAAKEFIAWLVRGRGK |
| 399 | HAEGTFTSDVSSYLEGQAAREFIAWLVRGKGK |
| 400 | HAEGTFTSDVSSYLEGQAAREFIAWLVRGRGK |
| 401 | HDEFERHAEGTFTSDVSSYLEGQAAREFIAWLVKGRGRK |
| 402 | HDEFERHAEGTFTSDVSSYLEGQAAKEFIAWLVRGRGRK |
| 403 | HDEFERHAEGTFTSDVSSYLEGQAAREFIAWLVRGKGRK |
| 404 | HAEGTFTSDVSSYLEGQAAREFIAWLVKGRGRK |
| 405 | HAEGTFTSDVSSYLEGQAAKEFIAWLVRGRGRK |
| 406 | HAEGTFTSDVSSYLEGQAAREFIAWLVRGKGRK |
| 407 | HGEGTFTSDVSSYLEGQAAREFIAWLVKGRGK |
| 408 | HGEGTFTSDVSSYLEGQAAREFIAWLVRGKGK |

GLP native sequences may be described by several sequence motifs, which are presented below. Letters in brackets represent acceptable amino acids at each sequence position: [HVY] [AGISTV] [DEHQ] [AG] [ILMPSTV] [FLY] [DINST] [ADEKNST] [ADENSTV] [LMVY] [ANRSTY] [EHIKNQRST] [AHILMQVY] [LMRT] [ADEGKQS] [ADEGKNQSY] [AEIKLMQR] [AKQRSVY] [AILMQSTV] [GKQR] [DEKLQR] [FHLVWY] [ILV] [ADEGHIKNQRST] [ADEGNRSTW] [GILVW] [AIKLMQSV] [ADGIKNQRST] [GKRSY]. In addition, synthetic analogs of GLP-1 can be useful as fusion partners to a masking moiety (such as XTEN) to create a fusion composition with biological activity useful in treatment of glucose-related disorders. Further sequences homologous to Exendin-4 or GLP-1 may be found by standard homology searching techniques.

“GLP-2” means human glucagon like peptide-2 and sequence variants thereof having at least a portion of the biological activity of mature GLP-2. More particularly, GLP-2 is a 33 amino acid peptide, co-secreted along with GLP-1 from intestinal endocrine cells in the small and large intestine.

“IGF-1” or “Insulin-like growth factor 1” means the human IGF-1 protein and species and sequence variants thereof having at least a portion of the biological activity of mature IGF-1. IGF-1, which was once called somatomedin C, is a polypeptide protein anabolic hormone similar in molecular structure to insulin, and that modulates the action of growth hormone. IGF-1 consists of 70 amino acids and is produced primarily by the liver as an endocrine hormone as well as in target tissues in a paracrine/autocrine fashion. IGF-1-containing fusion proteins of the invention may find particular use in the treatment of diabetes and insulin-resistance disorders for glucose regulation. IGF-1 has been cloned and expressed in E. coli and yeast, as described in U.S. Pat. No. 5,324,639.

“IGF-2” or “Insulin-like growth factor 2” means the human IGF-2 protein and species and sequence variants thereof having at least a portion of the biological activity of mature IGF-2. IGF-2 is a polypeptide protein hormone similar in molecular structure to insulin, with a primary role as a growth-promoting hormone during gestation. IGF-2 has been cloned, as described in Bell G I, et al. Isolation of the human insulin-like growth factor genes: insulin-like growth factor II and insulin genes are contiguous. Proc Natl Acad Sci USA. 1985. 82(19):6450-4.

“INGAP”, or “islet neogenesis-associated protein”, or “pancreatic beta cell growth factor” means the human INGAP peptide and species and sequence variants thereof having at least a portion of the biological activity of mature INGAP. INGAP is capable of initiating duct cell proliferation, a prerequisite for islet neogenesis. INGAP-containing fusion proteins of the invention may find particular use in the treatment or prevention of diabetes and insulin-resistance disorders. INGAP has been cloned and expressed, as described in R Rafaeloff R, et al., Cloning and sequencing of the pancreatic islet neogenesis associated protein (INGAP) gene and its expression in islet neogenesis in hamsters. J Clin Invest. 1997. 99(9): 2100-2109.

“Intermedin” or “AFP-6” means the human intermedin peptide and species and sequence variants thereof having at least a portion of the biological activity of mature intermedin. Intermedin is a ligand for the calcitonin receptor-like receptor. Intermedin treatment leads to blood pressure reduction both in normal and hypertensive subjects, as well as the suppression of gastric emptying activity, and is implicated in glucose homeostasis. Intermedin-containing fusion proteins of the invention may find particular use in the treatment of diabetes, insulin-resistance disorders, and obesity. Intermedin peptides and variants have been cloned, as described in U.S. Pat. No. 6,965,013.

“Leptin” means the naturally occurring leptin from any species, as well as biologically active D-isoforms, or fragments and sequence variants thereof. Leptin plays a key role in regulating energy intake and energy expenditure, including appetite and metabolism. Leptin-containing fusion proteins of the invention may find particular use in the treatment of diabetes for glucose regulation, insulin-resistance disorders, and obesity. Leptin is the polypeptide product of the ob gene as described in the International Patent Pub. No. WO 96/05309. Leptin has been cloned, as described in U.S. Pat. No. 7,112,659, and leptin analogs and fragments in U.S. Pat. Nos. 5,521,283, 5,532,336, PCT/US96/22308 and PCT/US96/01471.

“Neuromedin” means the neuromedin family of peptides including neuromedin U and S peptides, and sequence variants thereof. The native active human neuromedin U peptide hormone is neuromedin-U25, particularly its amide form. Of particular interest are their processed active peptide hormones and analogs, derivatives and fragments thereof. Included in the neuromedin U family are various truncated or splice variants, e.g., FLFHYSKTQKLGKSNVVEELQSPFASQSRGYFLFRPRN (SEQ ID NO: 409). Exemplary of the neuromedin S family is human neuromedin S with the sequence ILQRGSGTAAVDFTKKDHTATWGRPFFLFRPRN (SEQ ID NO: 267), particularly its amide form. Neuromedin fusion proteins of the invention may find particular use in treating obesity, diabetes, reducing food intake, and other related conditions and disorders as described herein. Of particular interest are neuromedin modules combined with an amylin family peptide, an exendin peptide family or a GLP I peptide family module.

“Oxyntomodulin”, or “OXM” means human oxyntomodulin and species and sequence variants thereof having at least a portion of the biological activity of mature OXM. OXM is a 37 amino acid peptide produced in the colon that contains the 29 amino acid sequence of glucagon followed by an 8 amino acid carboxyterminal extension. OXM has been found to suppress appetite. OXM-containing fusion proteins of the invention may find particular use in the treatment of diabetes for glucose regulation, insulin-resistance disorders, obesity, and can be used as a weight loss treatment.

“PYY” means human peptide YY polypeptide and species and sequence variants thereof having at least a portion of the biological activity of mature PYY. PYY includes both the human full length, 36 amino acid peptide, PYY₁₋₃₆ and PYY₃₋₃₆ which have the PP fold structural motif. PYY inhibits gastric motility and increases water and electrolyte absorption in the colon. PYY may also suppress pancreatic secretion. PPY-containing fusion proteins of the invention may find particular use in the treatment of diabetes for glucose regulation, insulin-resistance disorders, and obesity. Analogs of PYY have been prepared, as described in U.S. Pat. Nos. 5,604,203, 5,574,010 and 7,166,575.

“Urocortin” means a human urocortin peptide hormone and sequence variants thereof having at least a portion of the biological activity of mature urocortin. There are three human urocortins: Ucn-1, Ucn-2 and Ucn-3. Further urocortins and analogs have been described in U.S. Pat. No. 6,214,797. Urocortins Ucn-2 and Ucn-3 have food-intake suppression, antihypertensive, cardioprotective, and inotropic properties. Ucn-2 and Ucn-3 have the ability to suppress the chronic HPA activation following a stressful stimulus such as dieting/fasting, and are specific for the CRF type 2 receptor and do not activate CRF-R1 which mediates ACTH release. Therapeutic agents comprising urocortin, e.g., Ucn-2 or Ucn-3, may be useful for vasodilation and thus for cardiovascular uses such as chronic heart failure. Urocortin-containing fusion proteins of the invention may also find particular use in treating or preventing conditions associated with stimulating ACTH release, hypertension due to vasodilatory effects, inflammation mediated via other than ACTH elevation, hyperthermia, appetite disorder, congestive heart failure, stress, anxiety, and psoriasis. Urocortin-containing fusion proteins may also be combined with a natriuretic peptide module, amylin family, and exendin family, or a GLP 1 family module to provide an enhanced cardiovascular benefit, e.g. treating CHF, as by providing a beneficial vasodilation effect.

### Metabolic Disease and Cardiovascular Proteins

Metabolic and cardiovascular diseases represent a substantial health care burden in most developed nations, with cardiovascular diseases remaining the number one cause of death and disability in the United States and most European countries. Metabolic diseases and disorders include a large variety of conditions affecting the organs, tissues, and circulatory system of the body. Chief amongst these is diabetes; one of the leading causes of death in the United States, as it results in pathology and metabolic dysfunction in both the vasculature, central nervous system, major organs, and peripheral tissues. Insulin resistance and hyperinsulinemia have also been linked with two other metabolic disorders that pose considerable health risks: impaired glucose tolerance and metabolic obesity. Impaired glucose tolerance is characterized by normal glucose levels before eating, with a tendency toward elevated levels (hyperglycemia) following a meal. These individuals are considered to be at higher risk for diabetes and coronary artery disease. Obesity is also a risk factor for the group of conditions called insulin resistance syndrome, or “Syndrome X,” as is hypertension, coronary artery disease (arteriosclerosis), and lactic acidosis, as well as related disease states. The pathogenesis of obesity is believed to be multifactorial but an underlying problem is that in the obese, nutrient availability and energy expenditure are not in balance until there is excess adipose tissue.

Dyslipidemia is a frequent occurrence among diabetics and subjects with cardiovascular disease; typically characterized by parameters such as elevated plasma triglycerides, low HDL (high density lipoprotein) cholesterol, normal to elevated levels of LDL (low density lipoprotein) cholesterol and increased levels of small dense, LDL particles in the blood. Dyslipidemia and hypertension is a main contributor to an increased incidence of coronary events, renal disease, and deaths among subjects with metabolic diseases like diabetes and cardiovascular disease.

Cardiovascular disease can be manifest by many disorders, symptoms and changes in clinical parameters involving the heart, vasculature and organ systems throughout the body, including aneurysms, angina, atherosclerosis, cerebrovascular accident (Stroke), cerebrovascular disease, congestive heart failure, coronary artery disease, myocardial infarction, reduced cardiac output and peripheral vascular disease, hypertension, hypotension, blood markers (e.g., C-reactive protein, BNP, and enzymes such as CPK, LDH, SGPT, SGOT), amongst others.

Most metabolic processes and many cardiovascular parameters are regulated by multiple peptides and hormones (“metabolic proteins”), and many such peptides and hormones, as well as analogues thereof, have found utility in the treatment of such diseases and disorders. However, the use of therapeutic peptides and/or hormones, even when augmented by the use of small molecule drugs, has met with limited success in the management of such diseases and disorders. In particular, dose optimization is important for drugs and biologics used in the treatment of metabolic diseases, especially those with a narrow therapeutic window. Hormones in general, and peptides involved in glucose homeostasis often have a narrow therapeutic window. The narrow therapeutic window, coupled with the fact that such hormones and peptides typically have a short half-life which necessitates frequent dosing in order to achieve clinical benefit, results in difficulties in the management of such patients. Therefore, there remains a need for therapeutics with broader therapeutic window and increased efficacy and safety in the treatment of metabolic diseases.

In some embodiments of the compositions, as disclosed herein in this disclosure, the biologically active peptide (BP) can comprise a biologically active metabolic protein, and the composition can have utility in the treatment of metabolic and cardiovascular diseases and disorders. The metabolic proteins can include any protein of biologic, therapeutic, or prophylactic interest or function that is useful for preventing, treating, mediating, or ameliorating a metabolic or cardiovascular disease, disorder or condition. Table 3d provides a non-limiting list of such sequences of metabolic BPs that can be encompassed by the compositions (e.g., the therapeutic agents) of the invention. In some embodiments of the compositions disclosed herein, where the biologically active moiety is a biologically active peptide (BP), the BP can comprise a peptide sequence that exhibits at least (about) 80% sequence identity (e.g., at least (about) 81%, at least (about) 82%, at least (about) 83%, at least (about) 84%, at least (about) 85%, at least (about) 86%, at least (about) 87%, at least (about) 88%, at least (about) 89%, at least (about) 90%, at least (about) 91%, at least (about) 92%, at least (about) 93%, at least (about) 94%, at least (about) 95%, at least (about) 96%, at least (about) 97%, at least (about) 98%, at least (about) 99%, or 100% sequence identity) to an amino acid sequence of a metabolic protein set forth in Table 3d.

TABLE 3d
Biologically Active Proteins Relating to Metabolic Disorders and Cardiology

| | SEQ ID | |
|---|---|---|
| Name of Protein | NO: | Sequence |
| Anti-CD3 | | See U.S. Pat. Nos. 5,885,573 and 6,491,916 |
| IL-1ra, human full length | 410 | MEICRGLRSHLITLLLFLFHSETICRPSGRKSSKMQAFRIWD |
| | | VNQKTFYLRNNQLVAGYLQGPNVNLEEKIDVVPIEPHALF |
| | | LGIHGGKMCLSCVKSGDETRLQLEAVNITDLSENRKQDKR |
| | | FAFIRSDSGPTTSFESAACPGWFLCTAMEADQPVSLTNMP |
| | | DEGVMVTKFYFQEDE |
| IL-1ra, Dog | 411 | METCRCPLSYLISFLLFLPHSETACRLGKRPCRMQAFRIWD |
| | | VNQKTFYLRNNQLVAGYLQGSNTKLEEKLDVVPVEPHAV |
| | | FLGIHGGKLCLACVKSGDETRLQLEAVNITDLSKNKDQDK |
| | | RFTFILSDSGPTTSFESAACPGWFLCTALEADRPVSLTNRPE |
| | | EAMMVTKFYFQKE |
| IL-1ra, Rabbit | 412 | MRPSRSTRRHLISLLLFLFHSETACRPSGKRPCRMQAFRIW |
| | | DVNQKTFYLRNNQLVAGYLQGPNAKLEERIDVVPLEPQLL |
| | | FLGIQRGKLCLSCVKSGDKMKLHLEAVNITDLGKNKEQD |
| | | KRFTFIRSNSGPTTTFESASCPGWFLCTALEADQPVSLTNTP |
| | | DDSIVVTKFYFQED |
| IL-1ra, Rat | 413 | MEICRGPYSHLISLLLILLFRSESAGHIPAGKRPCKMQAFRI |
| | | WDTNQKTFYLRNNQLIAGYLQGPNTKLEEKIDMVPIDFRN |
| | | VFLGIHGGKLCLSCVKSGDDTKLQLEEVNITDLNKNKEED |
| | | KRFTFIRSETGPTTSFESLACPGWFLCTTLEADHPVSLTNTP |
| | | KEPCTVTKFYFQED |
| IL-1ra, Mouse | 414 | MEICWGPYSHLISLLLILLFHSEAACRPSGKRPCKMQAFRI |
| | | WDTNQKTFYLRNNQLIAGYLQGPNIKLEEKIDMVPIDLHS |
| | | VFLGIHGGKLCLSCAKSGDDIKLQLEEVNITDLSKNKEEDK |
| | | RFTFIRSEKGPTTSFESAACPGWFLCTTLEADRPVSLTNTPE |
| | | EPLIVTKFYFQEDQ |
| Anakinra | 415 | MRPSGRKSSKMQAFRIWDVNQKTFYLRNNQLVAGYLQGP |
| | | NVNLEEKIDVVPIEPHALFLGIHGGKMCLSCVKSGDETRLQ |
| | | LEAVNITDLSENRKQDKRFAFIRSDSGPTTSFESAACPGWF |
| | | LCTAMEADQPVSLTNMPDEGVMVTKFYFQEDE |
| α-natriuretic peptide (ANP) | 416 | SLRRSSCFGGRMDRIGAQSGLGCNSFRY |
| β-natriuretic peptide, human | 417 | SPKMVQGSGGFGRKMDRISSSSGLGCKVLRRH |
| (BNP human) | | |
| Brain natriuretic | 418 | NSKMAHSSSCFGQKIDRIGAVSRLGCDGLRLF |
| peptide, Rat; (BNP Rat) | | |
| C-type natriuretic peptide | 419 | GLSKGCFGLKLDRIGSMSGLGC |
| (CNP, porcine) | | |
| Fibroblast growth factor 2 | 420 | PALPEDGGSGAFPPGHFKDPKRLYCKNGGFFLRIHPDGRV |
| (FGF-2) | | DGVREKSDPHIKLQLQAEERGVVSIKGVCANRYLAMKED |
| | | GRLLASKCVTDECFFFERLESNNYNTYRS RKYTSWYVAL |
| | | KRTGQYKLGS KTGPGQKAIL FLPMSAKS |
| TNF receptor (TNFR) | 421 | LPAQVAFTPYAPEPGSTCRLREYYDQTAQMCCSKCSPGQH |
| | | AKVFCTKTSDTVCDSCEDSTYTQLWNWVPECLSCGSRCSS |
| | | DQVETQACTREQNRICTCRPGWYCALSKQEGCRLCAPLR |
| | | KCRPGFGVARPGTETSDVVCKPCAPGTFSNTTSSTDICRPH |
| | | QICNVVAIPGNASMDAVCTSTSPTRSMAPGAVHLPQPVST |
| | | RSQHTQPTPEPSTAPSTSFLLPMGPSPPAEGSTGD |

“Anti-CD3” means a monoclonal antibody against the T cell surface protein CD3, species and sequence variants, and fragments thereof, including OKT3 (also called muromonab) and humanized anti-CD3 monoclonal antibody (hOKT31(Ala-Ala))(KC Herold et al., New England Journal of Medicine 346:1692-1698. 2002) Anti-CD3 prevents T-cell activation and proliferation by binding the T-cell receptor complex present on all differentiated T cells. Anti-CD3-containing fusion proteins of the invention may find particular use to slow new-onset Type 1 diabetes, including use of the anti-CD3 as a therapeutic effector as well as a targeting moiety for a second therapeutic BP in the composition of this disclosure. The sequences for the variable region and the creation of an anti-CD3 have been described in U.S. Pat. Nos. 5,885,573 and 6,491,916.

“IL-1ra” means the human IL-1 receptor antagonist protein and species and sequence variants thereof, including the sequence variant anakinra (Kineret®), having at least a portion of the biological activity of mature IL-1ra. Human IL-1ra is a mature glycoprotein of 152 amino acid residues. The inhibitory action of IL-1ra results from its binding to the type I IL-1 receptor. The protein has a native molecular weight of 25 kDa, and the molecule shows limited sequence homology to IL-1α (19%) and IL-1β (26%). Anakinra is a nonglycosylated, recombinant human IL-1ra and differs from endogenous human IL-1ra by the addition of an N-terminal methionine. A commercialized version of anakinra is marketed as Kineret®. It binds with the same avidity to IL-1 receptor as native IL-1ra and IL-1b, but does not result in receptor activation (signal transduction), an effect attributed to the presence of only one receptor binding motif on IL-1ra versus two such motifs on IL-1α and IL-1β. Anakinra has 153 amino acids and 17.3 kD in size, and has a reported half-life of approximately 4-6 hours.

Increased IL-1 production has been reported in patients with various viral, bacterial, fungal, and parasitic infections; intravascular coagulation; high-dose IL-2 therapy; solid tumors; leukemias; Alzheimer's disease; HIV-1 infection; autoimmune disorders; trauma (surgery); hemodialysis; ischemic diseases (myocardial infarction); noninfectious hepatitis; asthma; UV radiation; closed head injury; pancreatitis; peritonitis; graft-versus-host disease; transplant rejection; and in healthy subjects after strenuous exercise. There is an association of increased IL-1b production in patients with Alzheimer's disease and a possible role for IL 1 in the release of the amyloid precursor protein. IL-1 has also been associated with diseases such as type 2 diabetes, obesity, hyperglycemia, hyperinsulinemia, type 1 diabetes, insulin resistance, retinal neurodegenerative processes, disease states and conditions characterized by insulin resistance, acute myocardial infarction (AMI), acute coronary syndrome (ACS), atherosclerosis, chronic inflammatory disorders, rheumatoid arthritis, degenerative intervertebral disc disease, sarcoidosis, Crohn's disease, ulcerative colitis, gestational diabetes, excessive appetite, insufficient satiety, metabolic disorders, glucagonomas, secretory disorders of the airway, osteoporosis, central nervous system disease, restenosis, neurodegenerative disease, renal failure, congestive heart failure, nephrotic syndrome, cirrhosis, pulmonary edema, hypertension, disorders wherein the reduction of food intake is desired, irritable bowel syndrome, myocardial infarction, stroke, post-surgical catabolic changes, hibernating myocardium, diabetic cardiomyopathy, insufficient urinary sodium excretion, excessive urinary potassium concentration, conditions or disorders associated with toxic hypervolemia, polycystic ovary syndrome, respiratory distress, chronic skin ulcers, nephropathy, left ventricular systolic dysfunction, gastrointestinal diarrhea, postoperative dumping syndrome, irritable bowel syndrome, critical illness polyneuropathy (CIPN), systemic inflammatory response syndrome (SIRS), dyslipidemia, reperfusion injury following ischemia, and coronary heart disease risk factor (CHDRF) syndrome. IL-1ra-containing fusion proteins of the invention may find particular use in the treatment of any of the foregoing diseases and disorders. IL-1ra has been cloned, as described in U.S. Pat. Nos. 5,075,222 and 6,858,409.

“Natriuretic peptides” means atrial natriuretic peptide (ANP), brain natriuretic peptide (BNP or B-type natriuretic peptide) and C-type natriuretic peptide (CNP); both human and non-human species and sequence variants thereof having at least a portion of the biological activity of the mature counterpart natriuretic peptides. Alpha atrial natriuretic peptide (aANP) or (ANP) and brain natriuretic peptide (BNP) and type C natriuretic peptide (CNP) are homologous polypeptide hormones involved in the regulation of fluid and electrolyte homeostasis. Sequences of useful forms of natriuretic peptides are disclosed in U.S. Patent Publication 20010027181. Examples of ANPs include human ANP (Kangawa et al., BBRC 118:131 (1984)) or that from various species, including pig and rat ANP (Kangawa et al., BBRC 121:585 (1984)). Sequence analysis reveals that preproBNP consists of 134 residues and is cleaved to a 108-amino acid ProBNP. Cleavage of a 32-amino acid sequence from the C-terminal end of ProBNP results in human BNP (77-108), which is the circulating, physiologically active form. The 32-amino acid human BNP involves the formation of a disulfide bond (Sudoh et al., BBRC 159:1420 (1989)) and U.S. Pat. Nos. 5,114,923, 5,674,710, 5,674,710, and 5,948,761. Compositions-containing one or more natriuretic functions may be useful in treating hypertension, diuresis inducement, natriuresis inducement, vascular conduct dilatation or relaxation, natriuretic peptide receptors (such as NPR-A) binding, 112apida secretion suppression from the kidney, aldostrerone secretion suppression from the adrenal gland, treatment of cardiovascular diseases and disorders, reducing, stopping or reversing cardiac remodeling after a cardiac event or as a result of congestive heart failure, treatment of renal diseases and disorders; treatment or prevention of ischemic stroke, and treatment of asthma.

“FGF-2” or heparin-binding growth factor 2, means the human FGF-2 protein, and species and sequence variants thereof having at least a portion of the biological activity of the mature counterpart. FGF-2 had been shown to stimulate proliferation of neural stem cells differentiated into striatal-like neurons and protect striatal neurons in toxin-induced models of Huntington Disease, and also my have utility in treatment of cardiac reperfusion injury, and may have endothelial cell growth, anti-angiogenic and tumor suppressive properties, wound healing, as well as promoting fracture healing in bones. FGF-2 has been cloned, as described in Burgess, W. H. and Maciag, T., Ann. Rev. Biochem., 58:575-606 (1989); Coulier, F., et al., 1994, Prog. Growth Factor Res. 5:1; and the PCT publication WO 87/01728.

“TNF receptor” means the human receptor for TNF, and species and sequence variants thereof having at least a portion of the biological receptor activity of mature TNFR. P75 TNF Receptor molecule is the extracellular domain of p75 TNF receptor, which is from a family of structurally homologous receptors which includes the p55 TNF receptor. TNFα and TNFβ (TNF ligands) compete for binding to the p55 and p75 TNF receptors. The x-ray crystal structure of the complex formed by the extracellular domain of the human p55 TNF receptor and TNFβ has been determined (Banner et al. Cell 73:431, 1993, incorporated herein by reference).

### Growth Hormone Proteins

“Growth Hormone” or “GH” means the human growth hormone protein and species and sequence variants thereof, and includes, but is not limited to, the 191 single-chain amino acid human sequence of GH. Thus, GH can be the native, full-length protein or can be a truncated fragment or a sequence variant that retains at least a portion of the biological activity of the native protein. Effects of GH on the tissues of the body can generally be described as anabolic. Like most other protein hormones, GH acts by interacting with a specific plasma membrane receptor, referred to as growth hormone receptor. There are two known types of human GH (hereinafter “hGH”) derived from the pituitary gland: one having a molecular weight of about 22,000 daltons (22 kD hGH) and the other having a molecular weight of about 20,000 daltons (20 kD hGH). The 20 kD HGH has an amino acid sequence that corresponds to that of 22 kD hGH consisting of 191 amino acids except that 15 amino acid residues from the 32^{nd} to the 46^{th} of 22 kD hGH are missing. Some reports have shown that the 20 kD hGH has been found to exhibit lower risks and higher activity than 22 kD hGH. The invention also contemplates use of the 20 kD hGH as being appropriate for use as a biologically active polypeptide for the compositions of this disclosure.

The invention contemplates inclusion in the compositions of any GH homologous sequences, sequence fragments that are natural, such as from primates, mammals (including domestic animals), and non-natural sequence variants which retain at least a portion of the biologic activity or biological function of GH and/or that are useful for preventing, treating, mediating, or ameliorating a GH-related disease, deficiency, disorder or condition. Non-mammalian GH sequences are well-described in the literature. For example, a sequence alignment of fish GHs can be found in Genetics and Molecular Biology 2003 26 p. 295-300. An analysis of the evolution of avian GH sequences is presented in Journal of Evolutionary Biology 2006 19 p. 844-854. In addition, native sequences homologous to human GH may be found by standard homology searching techniques, such as NCBI BLAST.

In one embodiment, the GH incorporated into the subject compositions can be a recombinant polypeptide with a sequence corresponding to a protein found in nature. In another embodiment, the GH can be a sequence variant, fragment, homolog, or a mimetics of a natural sequence that retains at least a portion of the biological activity of the native GH. Table 3f provides a non-limiting list of sequences of GHs from a wide variety of mammalian species that are encompassed by the compositions of this disclosure. Any of these GH sequences or homologous derivatives constructed by shuffling individual mutations between species or families may be useful for the fusion proteins of this invention. In some embodiments of the compositions disclosed herein, where the biologically active moiety can be a biologically active peptide (BP), the BP can comprise a peptide sequence that exhibits at least (about) 80% sequence identity (e.g., at least (about) 81%, at least (about) 82%, at least (about) 83%, at least (about) 84%, at least (about) 85%, at least (about) 86%, at least (about) 87%, at least (about) 88%, at least (about) 89%, at least (about) 90%, at least (about) 91%, at least (about) 92%, at least (about) 93%, at least (about) 94%, at least (about) 95%, at least (about) 96%, at least (about) 97%, at least (about) 98%, at least (about) 99%, or 100% sequence identity) to an amino acid sequence of a growth hormone set forth in Table 3f.

TABLE 3f
Growth Hormone Amino Acid Sequences from Animal Species

| | SEQ | |
|---|---|---|
| Species GH | ID NO: | Amino Acid Sequence |
| Man | 422 | FPTIPLSRLFDNAMLRAHRLHQLAFDTYQEFEEAYIPKEQKYSFLQNPQTSL |
| | | CFSESIPTPSNREETQQKSNLELLRISLLLIQSWLEPVQFLRSVFANSLVYGAS |
| | | DSNVYDLLKDLEEGIQTLMGRLEDGSPRTGQIFKQTYSKFDTNSHNDDALL |
| | | KNYGLLYCFRKDMDKVETFLRIVQCRSVEGSCGF |
| Pig | 423 | FPAMPLSSLFANAVLRAQHLHQLAADTYKEFERAYIPEGQRYSIQNAQAAF |
| | | CFSETIPAPTGKDEAQQRSDVELLRFSLLLIQSWLGPVQFLSRVFTNSLVFGT |
| | | SDRVYEKLKDLEEGIQALMRELEDGSPRAGQILKQTYDKFDTNLRSDDALL |
| | | KNYGLLSCFKKDLHKAETYLRV MKCRRFVESSCAF |
| Alpaca | 424 | FPAMPLSSLFANAVLRAQHLHQLAADTYKEFERTYIPEGQRYSIQNAQAAF |
| | | CFSETIPAPTGKDEAQQRSDVELLRFSLLLIQSWLGPVQFLSRVFTNSLVFGT |
| | | SDRVYEKLKDLEEGIQALMRELEDGSPRAGQILRQTYDKFDTNLRSDDALL |
| | | KNYGLLSCFKKDLHKAETYLRV MKCRRFVESSCAF |
| Camel | 425 | FPAMPLSSLFANAVLRAQHLHQLAADTYKEFERTYIPEGQRYSIQNAQAAF |
| | | CFSETIPAPTGKDEAQQRSDVELLRFSLLLIQSWLGPVQFLSRVFTNSLVFGT |
| | | SDRVYEKLKDLEEGIQALMRELEDGSPRAGQILRQTYDKFDTNLRSDDALL |
| | | KNYGLLSCFKKDLHKAETYLRV MKCRRFVESSCAF |
| Horse | 426 | FPAMPLSSLFANAVLRAQHLHQLAADTYKEFERAYIPEGQRYSIQNAQAAF |
| | | CFSETIPAPTGKDEAQQRSDMELLRFSLLLIQSWLGPVQLLSRVFTNSLVFG |
| | | TSDRVYEKLRDLEEGIQALMRELEDGSPRAGQILKQTYDKFDTNLRSDDAL |
| | | LKNYGLLSCFKKDLHKAETYLRV MKCRRFVESSCAF |
| Elephant | 427 | FPAMPLSSLFANAVLRAQHLHQLAADTYKEFERAYIPEGQRYSIQNAQAAF |
| | | CFSETIPAPTGKDEAQQRSDVELLRFSLLLIQSWLGPVQFLSRVFTNSLVFGT |
| | | SDRVYEKLKDLEEGIQALMRELEDGSPRPGQVLKQTYDKFDTNMRSDDAL |
| | | LKNYGLLSCFKKDLHKAETYLRV MKCRRFVESSCAF |
| Red fox | 428 | FPAMPLSSLFANAVLRAQHLHQLAADTYKEFERAYIPEGQRYSIQNAQAAF |
| | | CFSETIPAPTGKDEAQQRSDVELLRFSLVLIQSWLGPLQFLSRVFTNSLVFGT |
| | | SDRVYEKLKDLEEGIQALMRELEDGSPRAGQILKQTYDKFDTNLRSDDALL |
| | | KNYGLLSCFKKDLHKAETYLRV MKCRRFVESSCAF |
| Dog | 429 | FPAMPLSSLFANAVLRAQHLHQLAADTYKEFERAYIPEGQRYSIQNAQAAF |
| | | CFSETIPAPTGKDEAQQRSDVELLRFSLLLIQSWLGPVQFLSRVFTNSLVFGT |
| | | SDRVYEKLKDLEEGIQALMRELEDGSPRAGQILKQTYDKFDTNLRSDDALL |
| | | KNYGLLSCFKKDLHKAETYLRV MKCRRFVESSCAF |
| Cat | 430 | FPAMPLSSLFANAVLRAQHLHQLAADTYKEFERAYIPEGQRYSIQNAQAAF |
| | | CFSETIPAPTGKDEAQQRSDVELLRFSLLLIQSWLGPVQFLSRVFTNSLVFGT |
| | | SDRVYEKLKDLEEGIQALMRELEDGSPRGGQILKQTYDKFDTNLRSDDALL |
| | | KNYGLLSCFKKDLHKAETYLRV MKCRRFVESSCAF |
| American | 431 | FPAMPLSSLFANAVLRAQHLHQLAADTYKDFERAYIPEGQRYSIQNAQAAF |
| mink | | CFSETIPAPTGKDEAQQRSDMELLRFSLLLIQSWLGPVQFLSRVFTNSLVFGT |
| | | SDRVYEKLKDLEEGIQALMRELEDGSPRAGPILKQTYDKFDTNLRSDDALL |
| | | KNYGLLSCFKKDLHKAETYLRV MKCRRFVESSCAF |
| Finback | 432 | FPAMPLSSLFANAVLRAQHLHQLAADTYKEFERAYIPEGQRYSIQNAQAAF |
| whale | | CFSETIPAPTGKDEAQQRSDVELLRFSLLLIQSWLGPVQFLSRVFTNSLVFGT |
| | | SDRVYEKLKDLEEGIQALMRELEDGSPRAGQILKQTYDKFDTNMRSDDAL |
| | | LKNYGLLSCFKKDLHKAETYLRV MKCRRFVESSCAF |
| Dolphin | 433 | FPAMPLSSLFANAVLRAQHLHQLAADTYKEFERAYIPEGQRYSIQNTQAAF |
| | | CFSETIPAPTGKDEAQQRSDVELLRFSLLLIQSWLGPVQFLSRVFTNSLVFGT |
| | | SDRVYEKLKDLEEGIQALMRELEDGSPRAGQILKQTYDKFDTNMRSDDAL |
| | | LKNYGLLSCFKKDLHKAETYLRV MKCRRFVESSCAF |
| Hippo | 434 | FPAMPLSSLFANAVLRAQHLHQLAADTYKEFERAYIPEGQRYSIQNTQAAF |
| | | CFSETIPAPTGKDEAQQRSDVELLRFSLLLIQSWLGPVQFLSRVFTNSLVFGT |
| | | SDRVYEKLKDLEEGIQALMRELEDGSPRAGQILKQTYDKFDTNMRSDDAL |
| | | LKNYGLLSCFKKDLHKAETYLRV MKCRRFVESSCAF |
| Rabbit | 435 | FPAMPLSSLFANAVLRAQHLHQLAADTYKEFERAYIPEGQRYSIQNAQAAF |
| | | CFSETIPAPTGKDEAQQRSDMELLRFSLLLIQSWLGPVQFLSRAFTNTLVFG |
| | | TSDRVYEKLKDLEEGIQALMRELEDGSPRVGQLLKQTYDKFDTNLRGDDA |
| | | LLKNYGLLSCFKKDLHKAETYLRV MKCRRFVESSCVF |
| Rat | 436 | FPAMPLSSLFANAVLRAQHLHQLAADTYKEFERAYIPEGQRYSIQNAQAAF |
| | | CFSETIPAPTGKEEAQQRTDMELLRFSLLLIQSWLGPVQFLSRIFTNSLMFGT |
| | | SDRVYEKLKDLEEGIQALMQELEDGSPRIGQILKQTYDKFDANMRSDDALL |
| | | KNYGLLSCFKKDLHKAETYLRV MKCRRFAESSCAF |
| Mouse | 437 | FPAMPLSSLFSNAVLRAQHLHQLAADTYKEFERAYIPEGQRYSIQNAQAAF |
| | | CFSETIPAPTGKEEAQQRTDMELLRFSLLLIQSWLGPVQFLSRIFTNSLMFGT |
| | | SDRVYEKLKDLEEGIQALMQELEDGSPRVGQILKQTYDKFDANMRSDDAL |
| | | LKNYGLLSCFKKDLHKAETYLRV MKCRRFVESSCAF |
| Hamster | 438 | FPAMPLSSLFANAVLRAQHLHQLAADTYKEFERAYIPEGQRYSIQNAQTAF |
| | | CFSETIPAPTGKEEAQQRSDMELLRFSLLLIQSWLGPVQFLSRIFTNSLMFGT |
| | | SDRVYEKLKDLEEGIQALMQELEDGSPRVGQILKQTYDKFDTNMRSDDAL |
| | | LKNYGLLSCFKKDLHKAETYLRV MKCRRFVESSCAF |
| Mole rat | 439 | FPAMPLSNLFANAVLRAQHLHQLAADTYKEFERAYIPEGQRYSIQNAQAAF |
| | | CFSETIPAPTGKEEAQQRSDMELLRFSLLLIQSWLGPVQFLSRVFTNSLVFGT |
| | | SDRVFEKLKDLEEGIQALMRELEDGSLRAGQLLKQTYDKFDTNMRSDDAL |
| | | LKNYGLLSCFKKDLHKAETYLRV MKCRRFVESSCAF |
| Guinea pig | 440 | FPAMPLSSLFGNAVLRAQHLHQLAADTYKEFERTYIPEGQRYSIHNTQTAF |
| | | CFSETIPAPTDKEEAQQRSDVELLHFSLLLIQSWLGPVQFLSRVFTNSLVFGT |
| | | SDRVYEKLKDLEEGIQALMRELEDGTPRAGQILKQTYDKFDTNLRSNDALL |
| | | KNYGLLSCFRKDLHRTETYLRV MKCRRFVESSCAF |
| Ox | 441 | AFPAMSLSGLFANAVLRAQHLHQLAADTFKEFERTYIPEGQRYSIQNTQVA |
| | | FCFSETIPAPTGKNEAQQKSDLELLRISLLLIQSWLGPLQFLSRVFTNSLVFGT |
| | | SDRVYEKLKDLEEGILALMRELEDGTPRAGQILKQTYDKFDTNMRSDDAL |
| | | LKNYGLLSCFRKDLHKTETYLRV MKCRRFGEASCAF |
| Sheep/Goat | 442 | AFPAMSLSGLFANAVLRAQHLHQLAADTFKEFERTYIPEGQRYSIQNTQVA |
| | | FCFSETIPAPTGKNEAQQKSDLELLRISLLLIQSWLGPLQFLSRVFTNSLVFGT |
| | | SDRVYEKLKDLEEGILALMRELEDVTPRAGQILKQTYDKFDTNMRSDDAL |
| | | LKNYGLLSCFRKDLHKTETYLRV MKCRRFGEASCAF |
| Red deer | 443 | FPAMSLSGLFANAVLRAQHLHQLAADTFKEFERTYIPEGQRYSIQNTQVAF |
| | | CFSETIPAPTGKNEAQQKSDLELLRISLLLIQSWLGPLQFLSRVFTNSLVFGTS |
| | | DRVYEKLKDLEEGILALMRELEDGTPRAGQILKQTYDKFDTNMRSDDALL |
| | | KNYGLLSCFRKDLHKTETYLRV MKCRRFGEASCAF |
| Giraffe | 444 | AFPAMSLSGLFANAVLRAQHLHQLAADTFKEFERTYIPEGQRYSIQNTQVA |
| | | FCFSETIPAPTGKNEAQQKSDLELLRISLLLIQSWLGPLQFLSRVFSNSLVFGT |
| | | SDRVYEKLKDLEEGILALMRELEDGTPRAGQILKQTYDKFDTNMRSDDAL |
| | | LKNYGLLSCFRKDLHKTETYLRV MKCRRFGEASCAF |
| Chevrotain- | 445 | FPAMSLSGLFANAVLRVQHLHQLAADTFKEFERTYIPEGQRYSIQNTQVAF |
| 1 | | CFSETIPAPTGKNEAQQKSDLELLRISLLLIQSWLGPLQFLSRVFTNSLVFGTS |
| | | DRVYEKLKDLEEGILALMRELEDGPPRAGQILKQTYDKFDTNMRSDDALL |
| | | KNYGLLSCFRKDLHKTETYLRV MKCRRFGEASCAF |
| Slow loris | 446 | FPAMPLSSLFANAVLRAQHLHQLAADTYKEFERAYIPEGQRYSIQNAQAAF |
| | | CFSETIPAPTGKDEAQQRSDMELLRFSLLLIQSWLGPVQLLSRVFTNSLVLG |
| | | TSDRVYEKLKDLEEGIQALMRELEDGSPRVGQILKQTYDKFDTNLRSDDAL |
| | | LKNYGLLSCFKKDLHKAETYLRV MKCRRFVESSCAF |
| Marmoset | 447 | FPTIPLSRLLDNAMLRAHRLHQLAFDTYQEFEEAYIPKEQKYSFLQNPQTSL |
| | | CFSESIPTPASKKETQQKSNLELLRMSLLLIQSWFEPVQFLRSVFANSLLYGV |
| | | SDSDVYEYLKDLEEGIQTLMGRLEDGSPRTGEIFMQTYRKFDVNSQNNDAL |
| | | LKNYGLLYCFRKDMDKVETFLRI VQCR-SVEGSCGF |
| BrTailed | 448 | FPAMPLSSLFANAVLRAQHLHQLVADTYKEFERTYIPEAQRHSIQSTQTAFC |
| Possum | | FSETIPAPTGKDEAQQRSDVELLRFSLLLIQSWLSPVQFLSRVFTNSLVFGTS |
| | | DRVYEKLRDLEEGIQALMQELEDGSSRGGLVLKTTYDKFDTNLRSDEALL |
| | | KNYGLLSCFKKDLHKAETYLRV MKCRRFVESSCAF |
| Monkey | 449 | FPTIPLSRLFDNAMLRAHRLHQLAFDTYQEFEEAYIPKEQKYSFLQNPQTSL |
| (rhesus) | | CFSESIPTPSNREETQQKSNLELLRISLLLIQSWLEPVQFLRSVFANSLVYGTS |
| | | YSDVYDLLKDLEEGIQTLMGRLEDGSSRTGQIFKQTYSKFDTNSHNNDALL |
| | | KNYGLLYCFRKDMDKIETFLRI VQCR-SVEGSCGF |

### Cytokines

The BP can be a cytokine. Cytokines encompassed by the inventive compositions can have utility in the treatment in various therapeutic or disease categories, including but not limited to cancer, rheumatoid arthritis, multiple sclerosis, myasthenia gravis, systemic lupus erythematosus, Alzheimer's disease, Schizophrenia, viral infections (e.g., chronic hepatitis C, AIDS), allergic asthma, retinal neurodegenerative processes, metabolic disorder, insulin resistance, and diabetic cardiomyopathy. Cytokines can be especially useful in treating inflammatory conditions and autoimmune conditions.

The BP can be one or more cytokines. The cytokines refer to proteins (e.g., chemokines, interferons, lymphokines, interleukins, and tumor necrosis factors) released by cells which can affect cell behavior. Cytokines can be produced by a broad range of cells, including immune cells such as macrophages, B lymphocytes, T lymphocytes and mast cells, as well as endothelial cells, fibroblasts, and various stromal cells. A given cytokine can be produced by more than one type of cell. Cytokines can be involved in producing systemic or local immunomodulatory effects.

Certain cytokines can function as pro-inflammatory cytokines. Pro-inflammatory cytokines refer to cytokines involved in inducing or amplifying an inflammatory reaction. Pro-inflammatory cytokines can work with various cells of the immune system, such as neutrophils and leukocytes, to generate an immune response. Certain cytokines can function as anti-inflammatory cytokines. Anti-inflammatory cytokines refer to cytokines involved in the reduction of an inflammatory reaction. Anti-inflammatory cytokines, in some cases, can regulate a pro-inflammatory cytokine response. Some cytokines can function as both pro- and anti-inflammatory cytokines.

Examples of cytokines that are regulatable by systems and compositions of the present disclosure include, but are not limited to lymphokines, monokines, and traditional polypeptide hormones except for human growth hormone. Included among the cytokines are parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha; mullerian-inhibiting substance; mouse gonadotropin-associated peptide; inhibin; activin; vascular endothelial growth factor; integrin; thrombopoietin (TPO); nerve growth factors such as NGF-alpha; platelet-growth factor; transforming growth factors (TGFs) such as TGF-alpha, TGF-beta, TGF-beta1, TGF-beta2, and TGF-beta3; insulin-like growth factor-I and —II; erythropoietin (EPO); Flt-3L; stem cell factor (SCF); osteoinductive factors; interferons (IFNs) such as IFN-α, IFN-β, IFN-γ; colony stimulating factors (CSFs) such as macrophage-CSF (M-CSF); granulocyte-macrophage-CSF (GM-CSF); granulocyte-CSF (G-CSF); macrophage stimulating factor (MSP); interleukins (ILs) such as IL-1, IL-1a, IL-1b, IL-IRA, IL-18, IL-2, IL-3, IL-4, IL-5, IL-6, IL-7, IL-8, IL-9, IL-10, IL-11, IL-12, IL-12b, IL-13, IL-14, IL-15, IL-16, IL-17, IL-20; a tumor necrosis factor such as CD154, LT-beta, TNF-alpha, TNF-beta, 4-1BBL, APRIL, CD70, CD153, CD178, GITRL, LIGHT, OX40L, TALL-1, TRAIL, TWEAK, TRANCE; and other polypeptide factors including LIF, oncostatin M (OSM) and kit ligand (KL). Cytokine receptors refer to the receptor proteins which bind cytokines. Cytokine receptors may be both membrane-bound and soluble.

The target polynucleotide can encode for a cytokine. Non-limiting examples of cytokines include 4-1BBL, activin βA, activin βB, activin βC, activin 13E, artemin (ARTN), BAFF/BLyS/TNFSF138, BMP10, BMP15, BMP2, BMP3, BMP4, BMP5, BMP6, BMP7, BMP8a, BMP8b, bone morphogenetic protein 1 (BMP1), CCL1/TCA3, CCL11, CCL12/MCP-5, CCL13/MCP-4, CCL14, CCL15, CCL16, CCL17/TARC, CCL18, CCL19, CCL2/MCP-1, CCL20, CCL21, CCL22/MDC, CCL23, CCL24, CCL25, CCL26, CCL27, CCL28, CCL3, CCL3L3, CCL4, CCL4L1/LAG-1, CCL5, CCL6, CCL7, CCL8, CCL9, CD153/CD30L/TNFSF8, CD40L/CD154/TNFSF5, CD40LG, CD70, CD70/CD27L/TNFSF7, CLCF1, c-MPL/CD110/TPOR, CNTF, CX3CL1, CXCL1, CXCL10, CXCL11, CXCL12, CXCL13, CXCL14, CXCL15, CXCL16, CXCL17, CXCL2/MIP-2, CXCL3, CXCL4, CXCL5, CXCL6, CXCL7/Ppbp, CXCL9, EDA-A1, FAM19A1, FAM19A2, FAM19A3, FAM19A4, FAM19A5, Fas Ligand/FASLG/CD95L/CD178, GDF10, GDF11, GDF15, GDF2, GDF3, GDF4, GDF5, GDF6, GDF7, GDF8, GDF9, glial cell line-derived neurotrophic factor (GDNF), growth differentiation factor 1 (GDF1), IFNA1, IFNA10, IFNA13, IFNA14, IFNA2, IFNA4, IFNA5/IFNaG, IFNA7, IFNA8, IFNB1, IFNE, IFNG, IFNZ, IFNω/IFNW1, IL11, IL18, IL18BP, ILIA, IL1B, IL1F10, IL1F3/IL1RA, IL1F5, IL1F6, IL1F7, IL1F8, IL1F9, IL1RL2, IL31, IL33, IL6, IL8/CXCL8, inhibin-A, inhibin-B, Leptin, LIF, LTA/TNFB/TNFSF1, LTB/TNFC, neurturin (NRTN), OSM, OX-40L/TNFSF4/CD252, persephin (PSPN), RANKL/OPGL/TNFSF11(CD254), TL1A/TNFSF15, TNFA, TNF-alpha/TNFA, TNFSF10/TRAIL/APO-2L(CD253), TNFSF12, TNFSF13, TNFSF14/LIGHT/CD258, XCL1, and XCL2. In some embodiments, the target gene encodes for an immune checkpoint inhibitor. Non-limiting examples of such immune checkpoint inhibitors include PD-1, CTLA-4, LAG3, TIM-3, A2AR, B7-H3, B7-H4, BTLA, IDO, KIR, and VISTA. In some embodiments, the target gene encodes for a T cell receptor (TCR) alpha, beta, gamma, and/or delta chain.

In some cases, the cytokine can be a chemokine. The chemokine can be selected from a group including, but not limited to, ARMCX2, BCA-1/CXCL13, CCL11, CCL12/MCP-5, CCL13/MCP-4, CCL15/MIP-5/MIP-1 delta, CCL16/HCC-4/NCC4, CCL17/TARC, CCL18/PARC/MIP-4, CCL19/MIP-3b, CCL2/MCP-1, CCL20/MIP-3 alpha/MIP3A, CCL21/6Ckine, CCL22/MDC, CCL23/MIP 3, CCL24/Eotaxin-2/MPIF-2, CCL25/TECK, CCL26/Eotaxin-3, CCL27/CTACK, CCL28, CCL3/Mip1a, CCL4/MIP1B, CCL4L1/LAG-1, CCL5/RANTES, CCL6/C10, CCL8/MCP-2, CCL9, CML5, CXCL1, CXCL10/Crg-2, CXCL12/SDF-1 beta, CXCL14/BRAK, CXCL15/Lungkine, CXCL16/SR-PSOX, CXCL17, CXCL2/MIP-2, CXCL3/GRO gamma, CXCL4/PF4, CXCL5, CXCL6/GCP-2, CXCL9/MIG, FAM19A1, FAM19A2, FAM19A3, FAM19A4/TAFA4, FAM19A5, Fractalkine/CX3CL1, I-309/CCL1/TCA-3, IL-8/CXCL8, MCP-3/CCL7, NAP-2/PPBP/CXCL7, XCL2, and IL10.

Table 3g provides a non-limiting list of such sequences of BPs that are encompassed by the compositions of this disclosure. In some embodiments of the compositions disclosed herein, where the biologically active moiety can be a biologically active peptide (BP), the BP can comprise a peptide sequence that exhibits at least (about) 80% sequence identity (e.g., at least (about) 81%, at least (about) 82%, at least (about) 83%, at least (about) 84%, at least (about) 85%, at least (about) 86%, at least (about) 87%, at least (about) 88%, at least (about) 89%, at least (about) 90%, at least (about) 91%, at least (about) 92%, at least (about) 93%, at least (about) 94%, at least (about) 95%, at least (about) 96%, at least (about) 97%, at least (about) 98%, at least (about) 99%, or 100% sequence identity) to an amino acid sequence of a cytokine set forth in Table 3g.

TABLE 3g
Cytokines for Conjugation

| Name of | | |
|---|---|---|
| Protein | SEQ ID | |
| (Synonym) | NO: | Amino Acid Sequence |
| Anti-CD3 | | See U.S. Pat. Nos. 5,885,573 and 6,491,916 |
| IL-1ra, human | 450 | MEICRGLRSHLITLLLFLFHSETICRPSGRKSSKMQAFRIWDVNQKTFYLR |
| full length | | NNQLVAGYLQGPNVNLEEKIDVVPIEPHALFLGIHGGKMCLSCVKSGDE |
| | | TRLQLEAVNITDLSENRKQDKRFAFIRSDSGPTTSFESAACPGWFLCTAM |
| | | EADQPVSLTNMPDEGVMVTKFYFQEDE |
| IL-1ra, Dog | 451 | METCRCPLSYLISFLLFLPHSETACRLGKRPCRMQAFRIWDVNQKTFYLR |
| | | NNQLVAGYLQGSNTKLEEKLDVVPVEPHAVFLGIHGGKLCLACVKSGD |
| | | ETRLQLEAVNITDLSKNKDQDKRFTFILSDSGPTTSFESAACPGWFLCTAL |
| | | EADRPVSLTNRPEEAMMVTKFYFQKE |
| IL-1ra, Rabbit | 452 | MRPSRSTRRHLISLLLFLFHSETACRPSGKRPCRMQAFRIWDVNQKTFYL |
| | | RNNQLVAGYLQGPNAKLEERIDVVPLEPQLLFLGIQRGKLCLSCVKSGD |
| | | KMKLHLEAVNITDLGKNKEQDKRFTFIRSNSGPTTTFESASCPGWFLCTA |
| | | LEADQPVSLTNTPDDSIVVTKFYFQED |
| IL-1ra, Rat | 453 | MEICRGPYSHLISLLLILLFRSESAGHIPAGKRPCKMQAFRIWDTNQKTFY |
| | | LRNNQLIAGYLQGPNTKLEEKIDMVPIDFRNVFLGIHGGKLCLSCVKSGD |
| | | DTKLQLEEVNITDLNKNKEEDKRFTFIRSETGPTTSFESLACPGWFLCTTL |
| | | EADHPVSLTNTPKEPCTVTKFYFQED |
| IL-1ra, Mouse | 454 | MEICWGPYSHLISLLLILLFHSEAACRPSGKRPCKMQAFRIWDTNQKTFY |
| | | LRNNQLIAGYLQGPNIKLEEKIDMVPIDLHSVFLGIHGGKLCLSCAKSGD |
| | | DIKLQLEEVNITDLSKNKEEDKRFTFIRSEKGPTTSFESAACPGWFLCTTL |
| | | EADRPVSLTNTPEEPLIVTKFYFQEDQ |
| Anakinra | 455 | MRPSGRKSSKMQAFRIWDVNQKTFYLRNNQLVAGYLQGPNVNLEEKID |
| | | VVPIEPHALFLGIHGGKMCLSCVKSGDETRLQLEAVNITDLSENRKQDKR |
| | | FAFIRSDSGPTTSFESAACPGWFLCTAMEADQPVSLTNMPDEGVMVTKF |
| | | YFQEDE |
| IL-10 | 456 | MHSSALLCCLVLLTGVRASPGQGTQSENSCTHFPGNLPNMLRDLRDAFS |
| | | RVKTFFQMKDQLDNLLLKESLLEDFKGYLGCQALSEMIQFYLEEVMPQA |
| | | ENQDPDIKAHVNSLGENLKTLRLRLRRCHRFLPCENKSKAVEQVKNAFN |
| | | KLQEKGIYKAMSEFDIFINYIEAYMTMKIRN |

“IL-1ra” means the human IL-1 receptor antagonist protein and species and sequence variants thereof, including the sequence variant anakinra (Kineret®), having at least a portion of the biological activity of mature IL-1ra. Human IL-1ra is a mature glycoprotein of 152 amino acid residues. The inhibitory action of IL-1ra results from its binding to the type I IL-1 receptor. The protein has a native molecular weight of 25 kDa, and the molecule shows limited sequence homology to IL-1α (19%) and IL-1β (26%). Anakinra is a nonglycosylated, recombinant human IL-1ra and differs from endogenous human IL-1ra by the addition of an N-terminal methionine. A commercialized version of anakinra is marketed as Kineret®. It binds with the same avidity to IL-1 receptor as native IL-1ra and IL-1b, but does not result in receptor activation (signal transduction), an effect attributed to the presence of only one receptor binding motif on IL-1ra versus two such motifs on IL-1α and IL-1β. Anakinra has 153 amino acids and 17.3 kD in size, and has a reported half-life of approximately 4-6 hours.

Increased IL-1 production has been reported in patients with various viral, bacterial, fungal, and parasitic infections; intravascular coagulation; high-dose IL-2 therapy; solid tumors; leukemias; Alzheimer's disease; HIV-1 infection; autoimmune disorders; trauma (surgery); hemodialysis; ischemic diseases (myocardial infarction); noninfectious hepatitis; asthma; UV radiation; closed head injury; pancreatitis; peritonitis; graft-versus-host disease; transplant rejection; and in healthy subjects after strenuous exercise. There is an association of increased IL-1b production in patients with Alzheimer's disease and a possible role for IL 1 in the release of the amyloid precursor protein. IL-1 has also been associated with diseases such as type 2 diabetes, obesity, hyperglycemia, hyperinsulinemia, type 1 diabetes, insulin resistance, retinal neurodegenerative processes, disease states and conditions characterized by insulin resistance, acute myocardial infarction (AMI), acute coronary syndrome (ACS), atherosclerosis, chronic inflammatory disorders, rheumatoid arthritis, degenerative intervertebral disc disease, sarcoidosis, Crohn's disease, ulcerative colitis, gestational diabetes, excessive appetite, insufficient satiety, metabolic disorders, glucagonomas, secretory disorders of the airway, osteoporosis, central nervous system disease, restenosis, neurodegenerative disease, renal failure, congestive heart failure, nephrotic syndrome, cirrhosis, pulmonary edema, hypertension, disorders wherein the reduction of food intake is desired, irritable bowel syndrome, myocardial infarction, stroke, post-surgical catabolic changes, hibernating myocardium, diabetic cardiomyopathy, insufficient urinary sodium excretion, excessive urinary potassium concentration, conditions or disorders associated with toxic hypervolemia, polycystic ovary syndrome, respiratory distress, chronic skin ulcers, nephropathy, left ventricular systolic dysfunction, gastrointestinal diarrhea, postoperative dumping syndrome, irritable bowel syndrome, critical illness polyneuropathy (CIPN), systemic inflammatory response syndrome (SIRS), dyslipidemia, reperfusion injury following ischemia, and coronary heart disease risk factor (CHDRF) syndrome. IL-1ra-containing fusion proteins of the invention may find particular use in the treatment of any of the foregoing diseases and disorders. IL-1ra has been cloned, as described in U.S. Pat. Nos. 5,075,222 and 6,858,409.

In some cases, the BP can be IL-10. IL-10 can be an effective anti-inflammatory cytokine that represses the production of the proinflammatory cytokines and chemokines. IL-10 is the one of the major TH2-type cytokine that increases humoral immune responses and lowers cell-mediated immune reactions. IL-10 can be useful for the treatment of autoimmune diseases and inflammatory diseases such as rheumatoid arthritis, multiple sclerosis, myasthenia gravis, systemic lupus erythematosus, Alzheimer's, Schizophrenia, allergic asthma, retinal neurodegenerative processes, and diabetes.

In some cases, IL-10 can be modified to improve stability and decrease prolytic degradation. The modification can be one or more amide bond substitution. In some cases, one or more amide bonds within backbone of IL-10 can be substituted to achieve the abovementioned effects. The one or more amide linkages (—CONH—) in IL-10 can be replaced with a linkage which is an isostere of an amide linkage, such as —CH₂NH—, —CH₂S—, —CH₂CH₂—, —CH═CH— (cis and trans), —COCH₂—, —CH(OH)CH₂— or —CH₂SO—. Furthermore, the amide linkages in IL-10 can also be replaced by a reduced isostere pseudopeptide bond. See Couder et al. (1993) Int. J. Peptide Protein Res. 41:181-184, which is hereby incorporated by reference in its entirety.

The one or more acidic amino acids, including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids; and side chain amide residues such as asparagine, glutamine, and alkyl or aromatic substituted derivatives of asparagine or glutamine; as well as hydroxyl-containing amino acids, including serine, threonine, homoserine, 2,3-diaminopropionic acid, and alkyl or aromatic substituted derivatives of serine or threonine can be substituted.

The one or more hydrophobic amino acids in IL-10 such as alanine, leucine, isoleucine, valine, norleucine, (S)-2-aminobutyric acid, (5)-cyclohexylalanine or other simple alpha-amino acids can be substituted with amino acids including, but not limited to, an aliphatic side chain from C1-C10 carbons including branched, cyclic and straight chain alkyl, alkenyl or alkynyl substitutions

In some cases, the one or more hydrophobic amino acids in IL-10 such as can be substituted substitution of aromatic-substituted hydrophobic amino acids, including phenylalanine, tryptophan, tyrosine, sulfotyrosine, biphenylalanine, 1-naphthylalanine, 2-naphthylalanine, 2-benzothienylalanine, 3-benzothienylalanine, histidine, including amino, alkylamino, dialkylamino, aza, halogenated (fluoro, chloro, bromo, or iodo) or alkoxy (from C₁-C₄)-substituted forms of the above-listed aromatic amino acids, illustrative examples of which are: 2-, 3- or 4-aminophenylalanine, 2-, 3- or 4-chlorophenylalanine, 2-, 3- or 4-methylphenylalanine, 2-, 3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2, 3, or 4-biphenylalanine, 2′-, 3′-, or 4′-methyl-, 2-, 3- or 4-biphenylalanine, and 2- or 3-pyridylalanine;

The one or more hydrophobic amino acids in IL-10 such as phenylalanine, tryptophan, tyrosine, sulfotyrosine, biphenylalanine, 1-naphthylalanine, 2-naphthylalanine, 2-benzothienylalanine, 3-benzothienylalanine, histidine, including amino, alkylamino, dialkylamino, aza, halogenated (fluoro, chloro, bromo, or iodo) or alkox can be substituted by aromatic amino acids including: 2-, 3- or 4-aminophenylalanine, 2-, 3- or 4-chlorophenylalanine, 2-, 3- or 4-methylphenylalanine, 2-, 3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2, 3, or 4-biphenylalanine, 2′-, 3′-, or 4′-methyl-, 2-, 3- or 4-biphenylalanine, and 2- or 3-pyridylalanine

The amino acids comprising basic side chains, including arginine, lysine, histidine, ornithine, 2,3-diaminopropionic acid, homoarginine, including alkyl, alkenyl, or aryl-substituted derivatives of the previous amino acids, can be substituted. Examples are N-epsilon-isopropyl-lysine, 3-(4-tetrahydropyridyl)-glycine, 3-(4-tetrahydropyridyl)-alanine, N,N-gamma, gamma′-diethyl-homoarginine, alpha-methyl-arginine, alpha-methyl-2,3-diaminopropionic acid, alpha-methyl-histidine, and alpha-methyl-ornithine where the alkyl group occupies the pro-R position of the alpha-carbon. The modified IL-10 can comprise amides formed from any combination of alkyl, aromatic, heteroaromatic, ornithine, or 2,3-diaminopropionic acid, carboxylic acids or any of the many well-known activated derivatives such as acid chlorides, active esters, active azolides and related derivatives, lysine, and ornithine.

In some cases, IL-10 comprises can comprise one or more naturally occurring L-amino acids, synthetic L-amino acids, and/or D-enantiomers of an amino acid. The IL-10 polypeptide can comprise one or more of the following amino acids: ω-aminodecanoic acid, ω-aminotetradecanoic acid, cyclohexylalanine, α,γ-diaminobutyric acid, α,β-diaminopropionic acid, δ-amino valeric acid, t-butylalanine, t-butylglycine, N-methylisoleucine, phenylglycine, cyclohexylalanine, norleucine, naphthylalanine, ornithine, citrulline, 4-chlorophenylalanine, 2-fluorophenylalanine, pyridylalanine 3-benzothienyl alanine, hydroxyproline, β-alanine, o-aminobenzoic acid, m-aminobenzoic acid, p-aminobenzoic acid, m-aminomethylbenzoic acid, 2,3-diaminopropionic acid, α-aminoisobutyric acid, N-methylglycine(sarcosine), 3-fluorophenylalanine, 4-fluorophenylalanine, penicillamine, 1,2,3,4-tetrahydroisoquinoline-3-carboxylic acid, β-2-thienylalanine, methionine sulfoxide, homoarginine, N-acetyl lysine, 2,4-diamino butyric acid, rho-aminophenylalanine, N-methylvaline, homocysteine, homoserine, ε-amino hexanoic acid, ω-aminohexanoic acid, ω-aminoheptanoic acid, ω-aminooctanoic acid, and 2,3-diaminobutyric acid.

IL-10 can comprise a cysteine residue or a cysteine which can act as linker to another peptide via a disulfide linkage or to provide for cyclization of the IL-10 polypeptide. Methods of introducing a cysteine or cysteine analog are known in the art; see, e.g., U.S. Pat. No. 8,067,532. An IL-10 polypeptide can be cyclized. Other means of cyclization include introduction of an oxime linker or a lanthionine linker; see, e.g., U.S. Pat. No. 8,044,175. Any combination of amino acids (or non-amino acid moieties) that can form a cyclizing bond can be used and/or introduced. A cyclizing bond can be generated with any combination of amino acids (or with an amino acid and —(CH₂)ₙCO— or —(CH₂)ₙC₆H₄—CO—) with functional groups which allow for the introduction of a bridge. Some examples are disulfides, disulfide mimetics such as the —(CH₂)ₙ-carba bridge, thioacetal, thioether bridges (cystathionine or lanthionine) and bridges containing esters and ethers.

The IL-10 can be substituted with an N-alkyl, aryl, or backbone crosslinking to construct lactams and other cyclic structures, C-terminal hydroxymethyl derivatives, o-modified derivatives, N-terminally modified derivatives including substituted amides such as alkylamides and hydrazides. In some cases, an IL-10 polypeptide is a retroinverso analog.

IL-10 can be IL-10 can be native protein, peptide fragment IL-10, or modified peptide, having at least a portion of the biological activity of native IL-10. IL-10 can be modified to improve intracellular uptake. One such modification can be attachment of a protein transduction domain. The protein transduction domain can be attached to the C-terminus of the IL-10. Alternatively, the protein transduction domain can be attached to the N-terminus of the IL-10. The protein transduction domain can be attached to IL-10 via covalent bond. The protein transduction domain can be chosen from any of the sequences listed in Table 3h.

TABLE 3h
Exemplary protein transduction domains

| SEQ ID NO: | Amino Acid Sequence |
|---|---|
| 457 | YGRKKRRQRRR |
| 458 | RRQRRTSKLMKR |
| 459 | GWTLNSAGYLLGKINLKALAALAKKIL |
| 460 | KALAWEAKLAKALAKALAKHLAKALAKALKCEA |
| 461 | RQIKIWFQNRRMKWKK |
| 462 | YGRKKRRQRRR |
| 463 | RKKRRQRRR |
| 464 | YGRKKRRQRRR |
| 465 | RKKRRQRR |
| 466 | YARAAARQARA |
| 467 | THRLPRRRRRR |
| 468 | GGRRARRRRRR |

The BP of the subject compositions are not limited to native, full-length polypeptides, but also include recombinant versions as well as biologically and/or pharmacologically active variants or fragments thereof. For example, it will be appreciated that various amino acid substitutions can be made in the GP to create variants without departing from the spirit of the invention with respect to the biological activity or pharmacologic properties of the BP. Examples of conservative substitutions for amino acids in polypeptide sequences are shown in Table 4. However, in embodiments of the compositions of this disclosure in which the sequence identity of the BP is less than 100% compared to a specific sequence disclosed herein, the invention contemplates substitution of any of the other 19 natural L-amino acids for a given amino acid residue of the given BP, which may be at any position within the sequence of the BP, including adjacent amino acid residues. If any one substitution results in an undesirable change in biological activity, then one of the alternative amino acids can be employed and the construct evaluated by the methods described herein, or using any of the techniques and guidelines for conservative and non-conservative mutations set forth, for instance, in U.S. Pat. No. 5,364,934, the contents of which is incorporated by reference in its entirety, or using methods generally known to those of skill in the art. In addition, variants can also include, for instance, polypeptides wherein one or more amino acid residues are added or deleted at the N- or C-terminus of the full-length native amino acid sequence of a BP that retains at least a portion of the biological activity of the native peptide.

TABLE 4
Exemplary conservative amino acid substitutions

| Original Residue | Exemplary Substitutions |
|---|---|
| Ala (A) | val; leu; ile |
| Arg (R) | lys; gln; asn |
| Asn (N) | gln; his; Iys; arg |
| Asp (D) | glu |
| Cys (C) | ser |
| Gln (Q) | asn |
| Glu (E) | asp |
| Gly (G) | pro |
| His (H) | asn: gln: Iys: arg |
| xIle (I) | leu; val; met; ala; phe: norleucine |
| Leu (L) | norleucine: ile: val; met; ala: phe |
| Lys (K) | arg: gln: asn |
| Met (M) | leu; phe; ile |
| Phe (F) | leu: val: ile; ala |
| Pro (P) | gly |
| Ser (S) | thr |
| Thr (T) | ser |
| Trp (W) | tyr |
| Tyr(Y) | trp: phe: thr: ser |
| Val (V) | ile; leu; met; phe; ala; norleucine |

In some embodiments, a BP incorporated into a composition of this disclosure can have a sequence that exhibits at least (about) 80% (or at least (about) 81%, or at least (about) 82%, or at least (about) 83%, or at least (about) 84%, or at least (about) 85%, or at least (about) 86%, or at least (about) 87%, or at least (about) 88%, or at least (about) 89%, or at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99%, or (about) 100% sequence identity to a sequence from Tables 3a-3h. In some embodiments of the compositions of this disclosure, the sequence of the BP can comprise one or more substitutions shown in Table 4.

### Antibodies:

In some embodiments of the compositions of this disclosure, the biologically active peptide (BP) can comprise an antibody, such as a monospecific, bispecific, or multispecific antibody. The antibody can comprise a binding domain (or binding moiety) having specific binding affinity to a tumor-specific marker or an antigen of a target cell (or a target cell antigen) (such as one described more fully hereinbelow). The antibody can comprise a binding domain (or binding moiety) that binds to an effector cell antigen (such as one described more fully hereinbelow). In some embodiments of the compositions of this disclosure, the antibody, such as a bispecific or multi-specific antibody, can comprise (1) a binding domain (e.g., a first or second binding domain) having specific binding affinity to a tumor-specific marker or a target cell antigen (such as one described more fully hereinbelow) and (2) another binding domain (e.g., a second or first binding domain) that binds to an effector cell antigen (such as one described more fully hereinbelow). The disclosure contemplates use of single chain binding domains, such as but not limited to Fv, Fab, Fab′, Fab′-SH, nanobodies (also known as single domain antibodies or V_{HH}), F(ab′)2, linear antibodies, single domain antibody, single domain camelid antibody, single-chain antibody molecules (scFv), multispecific antibodies formed from antibody fragments, and diabodies capable of binding ligands or receptors associated with effector cells and antigens of diseased tissues or cells (such as cancers, tumors, or other malignant tissues). The binding domain (or the first binding domain, or the second binding domain) can be a non-antibody scaffold selected from anticalins, adnectins, fynomers, affilins, affibodies, centyrins, DARPins. The binding domain (or the first binding domain, or the second binding domain) for a tumor cell target can be a variable domain of a T cell receptor engineered to bind major histocompatibility complex (MHC) that is loaded with a peptide fragment of a protein that is overexpressed by tumor cells. In some embodiments of the compositions of this disclosure (such as XTENylated Protease-Activated T Cell Engagers (“XPAT” or “XPATs”), other masked therapeutic antibodies, etc.) the biologically active peptide (BP) can be a bispecific antibody (e.g., a bispecific T-cell engager).

With respect to single chain binding domains (or binding moieties), as is well established, an active antibody fragment (Fv) is the minimum antibody fragment which contains a complete antigen recognition and binding site; consisting of a dimer of one heavy (VH) and one light chain variable domain (VL) in non-covalent association. Each scFv can comprise one VL and one VH. Within each VH and VL chain are three complementarity determining regions (CDRs) that interact to define an antigen binding site on the surface of the VH-VL dimer; the six CDRs of a binding domain (or binding moiety) confer antigen binding specificity to the antibody or single chain binding domain (or binding moiety). In some cases, scFv are created in which each has 3, 4, or 5 CHRs within each binding domain (or binding moiety). Framework sequences flanking the CDRs have a tertiary structure that is essentially conserved in native immunoglobulins across species, and the framework residues (FR) serve to hold the CDRs in their appropriate orientation. The constant domains are not required for binding function, but may aid in stabilizing VH-VL interaction. In some embodiments, the domain of the binding site of the polypeptide can be a pair of VH-VL, VH-VH or VL-VL domains either of the same or of different immunoglobulins, however it is generally preferred to make single chain binding domains (or binding moieties) using the respective VH and VL chains from the parental antibody. The order of VH and VL domains within the polypeptide chain is not limiting for the present invention; the order of domains given may be reversed usually without any loss of function, but it is understood that the VH and VL domains are arranged so that the antigen binding site can properly fold. Thus, the single chain binding domains of the bispecific scFv embodiments of the subject compositions can be in the order (VL-VH)1-(VL-VH)2, wherein “1” and “2” represent the first and second binding domains (or the first and second binding moieties), respectively, or (VL-VH)1-(VH-VL)2, or (VH-VL)1-(VL-VH)2, or (VH-VL)1-(VH-VL)2, wherein the paired binding domains (or binding moieties) are linked by a polypeptide linker as described hereinbelow.

In some embodiments of the compositions, wherein the BP comprises (1) a binding domain (or binding moiety) having specific binding affinity to a tumor-specific marker or an antigen of a target cell (or a target cell antigen) and (2) a binding domain (or binding moiety) that binds to an effector cell antigen, the arrangement of the binding domains (or binding moieties) in an exemplary bispecific single chain antibody disclosed herein may therefore be one in which the first binding domain (or first binding moiety) can be located C-terminally to the second binding domain (or second binding moiety). The arrangement of the V chains can be VH (target cell surface antigen)-VL (target cell surface antigen)-VL (effector cell antigen)-VH (effector cell antigen), VH (target cell surface antigen)-VL (target cell surface antigen)-VH (effector cell antigen)-VL (effector cell antigen), VL (target cell surface antigen)-VH (target cell surface antigen)-VL (effector cell antigen)-VH (effector cell antigen) or VL (target cell surface antigen)-VH (target cell surface antigen)-VH (effector cell antigen)-VL (effector cell antigen). For an arrangement, in which the second binding domain (or second binding moiety) can be located N-terminally to the first binding domain (or first binding moiety), the following orders are possible: VH (effector cell antigen)-VL (effector cell antigen)-VL (target cell surface antigen)-VH (target cell surface antigen), VH (effector cell antigen)-VL (effector cell antigen)-VH (target cell surface antigen)-VL (target cell surface antigen), VL (effector cell antigen)-VH (effector cell antigen)-VL (target cell surface antigen)-VH (target cell surface antigen) or VL (effector cell antigen)-VH (effector cell antigen)-VH (target cell surface antigen)-VL (target cell surface antigen). As used herein, “N-terminally to” or “C-terminally to” and grammatical variants thereof denote relative location within the primary amino acid sequence rather than placement at the absolute N- or C-terminus of the bispecific single chain antibody. Hence, as a non-limiting example, a first binding domain (or first binding moiety) which is “located C-terminally to the second binding domain” denotes that the first binding is located on the carboxyl side of the second binding domain (or second binding moiety) within the bispecific single chain antibody, and does not exclude the possibility that an additional sequence, for example a His-tag, or another compound such as a radioisotope, is located at the C-terminus of the bispecific single chain antibody.

The VL and VH domains can be derived from monoclonal antibodies with binding specificity to the tumor-specific marker or the antigen of the target cell and effector cell antigens, respectively. In other cases, the first and second binding domains (or the first and second binding moieties) each comprise six CDRs derived from monoclonal antibodies with binding specificity to a target cell marker, such as a tumor-specific marker and effector cell antigens, respectively. In other embodiments, the first and second binding domains (or the first and second binding moieties) of the subject compositions can have 3, 4, or 5 CHRs within each binding domain (or each binding moiety). In other embodiments, the embodiments of the invention comprise a first binding domain and a second binding domain wherein each comprises a CDR-H1 region, a CDR-H2 region, a CDR-H3 region, a CDR-L1 region, a CDR-L2 region, and a CDR-H3 region, where each of the regions can be derived from a monoclonal antibody capable of binding the tumor-specific marker or the antigen of the target cell, and effector cell antigens, respectively.

In some embodiments, where the BP comprises a binding domain (or binding moiety) (or a first binding domain, or a second binding domain) having binding affinity for an effector cell antigen, the effector cell antigen can be expressed on the surface of an effector cell selected from a plasma cell, a T cell, a B cell, a cytokine induced killer cell (CIK cell), a mast cell, a dendritic cell, a regulatory T cell (RegT cell), a helper T cell, a myeloid cell, and a NK cell. The effector cell antigen can be expressed on or within an effector cell. The effector cell antigen can be expressed on a T cell, such as a CD4+, CD8+, or natural killer (NK) cell. The effector cell antigen can be expressed on the surface of a T cell. The effector cell antigen can be expressed on a B cell, master cell, dendritic cell, or myeloid cell.

In some embodiments of the compositions herein, the BP can comprise a binding domain (or binding moiety) (or a first binding domain, or a second binding domain) having specific binding affinity to a tumor-specific marker or an antigen of a target cell (or a target cell antigen). The tumor-specific marker or the target cell antigen can be associated with a tumor cell. The tumor cell can be of a tumor, such as stroma cell tumor, fibroblast tumor, myofibroblast tumor, glial cell tumor, epithelial cell tumor, fat cell tumor, immune cell tumor, vascular cell tumor, or smooth muscle cell tumor. The tumor-specific marker or the antigen of the target cell can be selected from the group consisting of alpha 4 integrin, Ang2, B7-H3, B7-H6 (e.g., its natural ligand Nkp30 rather than an antibody fragment), CEACAM5, cMET, CTLA4, FOLR1, EpCAM, CCR5, CD19, HER2, HER2 neu, HER3, HER4, HER1 (EGFR), PD-L1, PSMA, CEA, TROP-2, MUC1(mucin), MUC-2, MUC3, MUC4, MUC5AC, MUC5B, MUC7, MUC16 βhCG, Lewis-Y, CD20, CD33, CD38, CD30, CD56 (NCAM), CD133, ganglioside GD3; 9-O-Acetyl-GD3, GM2, Globo H, fucosyl GM1, GD2, carbonicanhydrase IX, CD44v6, Nectin-4, Sonic Hedgehog (Shh), Wue-1, plasma cell antigen 1, melanoma chondroitin sulfate proteoglycan (MCSP), CCR8, 6-transmembrane epithelial antigen of prostate (STEAP), mesothelin, A33 antigen, prostate stem cell antigen (PSCA), Ly-6, desmoglein 4, fetal acetylcholine receptor (fnAChR), CD25, cancer antigen 19-9 (CA19-9), cancer antigen 125 (CA-125), Muellerian inhibitory substance receptor type II (MISIIR), sialylated Tn antigen (s TN), fibroblast activation antigen (FAP), endosialin (CD248), epidermal growth factor receptor variant III (EGFRvIII), tumor-associated antigen L6 (TAL6), SAS, CD63, TAG72, Thomsen-Friedenreich antigen (TF-antigen), insulin-like growth factor I receptor (IGF-IR), Cora antigen, CD7, CD22, CD70 (e.g., its natural ligand, CD27 rather than an antibody fragment), CD79a, CD79b, G250, MT-MMPs, CA19-9, CA-125, alpha-fetoprotein (AFP), VEGFR1, VEGFR2, DLK1, SP17, ROR1, and EphA2. The tumor-specific marker or the antigen of the target cell can be selected from the group consisting of alpha 4 integrin, Ang2, B7-H3, B7-H6 (e.g., its natural ligand Nkp30 rather than an antibody fragment), CEACAM5, cMET, CTLA4, FOLR1, EpCAM (epithelial cell adhesion molecule), CCR5, CD19, HER2, HER2 neu, HER3, HER4, HER1 (EGFR), PD-L1, PSMA, CEA, TROP-2, MUC1(mucin), MUC-2, MUC3, MUC4, MUC5AC, MUC5B, MUC7, MUC16, βhCG, Lewis-Y, CD20, CD33, CD38, CD30, CD56 (NCAM), CD133, ganglioside GD3, 9-O-acetyl-GD3, GM2, Globo H, fucosyl GM1, GD2, carbonicanhydrase IX, CD44v6, Nectin-4, Sonic Hedgehog (Shh), Wue-1, plasma cell antigen 1 (PC-1), melanoma chondroitin sulfate proteoglycan (MCSP), CCR8, 6-transmembrane epithelial antigen of prostate (STEAP), mesothelin, A33 antigen, prostate stem cell antigen (PSCA), Ly-6, desmoglein 4, fetal acetylcholine receptor (fnAChR), CD25, cancer antigen 19-9 (CA19-9), cancer antigen 125 (CA-125), Muellerian inhibitory substance receptor type II (MISIIR), sialylated Tn antigen (sTN), fibroblast activation antigen (FAP), endosialin (CD248), epidermal growth factor receptor variant III (EGFRvIII), tumor-associated antigen L6 (TAL6), SAS, CD63, TAG72, Thomsen-Friedenreich antigen (TF-antigen), insulin-like growth factor I receptor (IGF-IR), Cora antigen, CD7, CD22, CD70 (e.g., its natural ligand, CD27 rather than an antibody fragment), CD79a, CD79b, G250, MT-MMPs, alpha-fetoprotein (AFP), VEGFR1, VEGFR2, DLK1, SP17, ROR1, EphA2, ENPP3, glypican 3 (GPC3), and TPBG/5T4 (trophoblast glycoprotein). The tumor-specific marker or the antigen of the target cell can be selected from alpha 4 integrin, Ang2, CEACAM5, cMET, CTLA4, FOLR1, EpCAM (epithelial cell adhesion molecule), CD19, HER2, HER2 neu, HER3, HER4, HER1 (EGFR), PD-L1, PSMA, CEA, TROP-2, MUC1(mucin), Lewis-Y, CD20, CD33, CD38, mesothelin, CD70 (e.g., its natural ligand, CD27 rather than an antibody fragment), VEGFR1, VEGFR2, ROR1, EphA2, ENPP3, glypican 3 (GPC3), and TPBG/5T4 (trophoblast glycoprotein). The VL and VH sequences of the binding domain (or binding moiety) (or the first binding domain, or the second binding domain) having specific binding affinity to a tumor-specific marker or an antigen of a target cell (or a target antigen) can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99%, or 100%, sequence identity to any one of the paired VL and VH sequences set forth in the “VH Sequences” and “VL Sequences” columns of Table 6 (as described more fully hereinbelow).

Therapeutic monoclonal antibodies from which VL and VH and CDR domains can be derived for the subject compositions are known in the art. Such therapeutic antibodies include, but are not limited to, rituximab, IDEC/Genentech/Roche (see for example U.S. Pat. No. 5,736,137), a chimeric anti-CD20 antibody used in the treatment of many lymphomas, leukemias, and some autoimmune disorders; ofatumumab, an anti-CD20 antibody approved for use for chronic lymphocytic leukemia, and under development for follicular non-Hodgkin's lymphoma, diffuse large B cell lymphoma, rheumatoid arthritis and relapsing remitting multiple sclerosis, being developed by GlaxoSmithKline; lucatumumab (HCD122), an anti-CD40 antibody developed by Novartis for Non-Hodgkin's or Hodgkin's Lymphoma (see, for example, U.S. Pat. No. 6,899,879), AME-133, an antibody developed by Applied Molecular Evolution which binds to cells expressing CD20 to treat non-Hodgkin's lymphoma, veltuzumab (hA20), an antibody developed by Immunomedics, Inc. which binds to cells expressing CD20 to treat immune thrombocytopenic purpura, HumaLYM developed by Intracel for the treatment of low-grade B-cell lymphoma, and ocrelizumab, developed by Genentech which is an anti-CD20 monoclonal antibody for treatment of rheumatoid arthritis (see for example U.S. Patent Application 20090155257), trastuzumab (see for example U.S. Pat. No. 5,677,171), a humanized anti-Her2/neu antibody approved to treat breast cancer developed by Genentech; pertuzumab, an anti-HER2 dimerization inhibitor antibody developed by Genentech in treatment of in prostate, breast, and ovarian cancers; (see for example U.S. Pat. No. 4,753,894); cetuximab, an anti-EGFR antibody used to treat epidermal growth factor receptor (EGFR)-expressing, KRAS wild-type metastatic colorectal cancer and head and neck cancer, developed by Imclone and BMS (see U.S. Pat. No. 4,943,533; PCT WO 96/40210); panitumumab, a fully human monoclonal antibody specific to the epidermal growth factor receptor (also known as EGF receptor, EGFR, ErbB-1 and HER1, currently marketed by Amgen for treatment of metastatic colorectal cancer (see U.S. Pat. No. 6,235,883); zalutumumab, a fully human IgG1 monoclonal antibody developed by Genmab that is directed towards the epidermal growth factor receptor (EGFR) for the treatment of squamous cell carcinoma of the head and neck (see for example U.S. Pat. No. 7,247,301); nimotuzumab, a chimeric antibody to EGFR developed by Biocon, YM Biosciences, Cuba, and Oncosciences, Europe) in the treatment of squamous cell carcinomas of the head and neck, nasopharyngeal cancer and glioma (see for example U.S. Pat. Nos. 5,891,996; 6,506,883); alemtuzumab, a humanized monoclonal antibody to CD52 marketed by Bayer Schering Pharma for the treatment of chronic lymphocytic leukemia (CLL), cutaneous T-cell lymphoma (CTCL) and T-cell lymphoma; muromonab-CD3, an anti-CD3 antibody developed by Ortho Biotech/Johnson & Johnson used as an immunosuppressant biologic given to reduce acute rejection in patients with organ transplants; ibritumomab tiuxetan, an anti-CD20 monoclonal antibody developed by IDEC/Schering AG as treatment for some forms of B cell non-Hodgkin's lymphoma; gemtuzumab ozogamicin, an anti-CD33 (p67 protein) antibody linked to a cytotoxic chelator tiuxetan, to which a radioactive isotope can be attached, developed by Celltech/Wyeth used to treat acute myelogenous leukemia; ABX-CBL, an anti-CD147 antibody developed by Abgenix; ABX-IL8, an anti-IL8 antibody developed by Abgenix, ABX-MA1, an anti-MUC18 antibody developed by Abgenix, Pemtumomab (R1549, 90Y-muHMFG1), an anti-MUC1 in development by Antisoma, Therex (R1550), an anti-MUC1 antibody developed by Antisoma, AngioMab (AS1405), developed by Antisoma, HuBC-1, developed by Antisoma, Thioplatin (AS1407) developed by Antisoma, ANTEGREN (natalizumab), an anti-alpha-4-beta-1 (VLA4) and alpha-4-beta-7 antibody developed by Biogen, VLA-1 mAb, an anti-VLA-1 integrin antibody developed by Biogen, LTBR mAb, an anti-lymphotoxin beta receptor (LTBR) antibody developed by Biogen, CAT-152, an anti-TGF-β2 antibody developed by Cambridge Antibody Technology, J695, an anti-IL-12 antibody developed by Cambridge Antibody Technology and Abbott, CAT-192, an anti-TGFβ1 antibody developed by Cambridge Antibody Technology and Genzyme, CAT-213, an anti-Eotaxin 1 antibody developed by Cambridge Antibody Technology, LYMPHOSTAT-B, an anti-Blys antibody developed by Cambridge Antibody Technology and Human Genome Sciences Inc., TRAIL-R1mAb, an anti-TRAIL-R1 antibody developed by Cambridge Antibody Technology and Human Genome Sciences, Inc.; HERCEPTIN, an anti-HER receptor family antibody developed by Genentech; Anti-Tissue Factor (ATF), an anti-Tissue Factor antibody developed by Genentech; XOLAIR (Omalizumab), an anti-IgE antibody developed by Genentech, MLN-02 Antibody (formerly LDP-02), developed by Genentech and Millennium Pharmaceuticals; HUMAX CD4®, an anti-CD4 antibody developed by Genmab; tocilizuma, and anti-IL6R antibody developed by Chugai; HUMAX-IL15, an anti-IL15 antibody developed by Genmab and Amgen, HUMAX-Inflam, developed by Genmab and Medarex; HUMAX-Cancer, an anti-Heparanase I antibody developed by Genmab and Medarex and Oxford GlycoSciences; HUMAX-Lymphoma, developed by Genmab and Amgen, HUMAX-TAC, developed by Genmab; IDEC-131, an anti-CD40L antibody developed by IDEC Pharmaceuticals; IDEC-151 (Clenoliximab), an anti-CD4 antibody developed by IDEC Pharmaceuticals; IDEC-114, an anti-CD80 antibody developed by IDEC Pharmaceuticals; IDEC-152, an anti-CD23 developed by IDEC Pharmaceuticals; an anti-KDR antibody developed by Imclone, DC101, an anti-flk-1 antibody developed by Imclone; anti-VE cadherin antibodies developed by Imclone; CEA-CIDE (labetuzumab), an anti-carcinoembryonic antigen (CEA) antibody developed by Immunomedics; Yervoy (ipilimumab), an anti-CTLA4 antibody developed by Bristol-Myers Squibb in the treatment of melanoma; Lumphocide® (Epratuzumab), an anti-CD22 antibody developed by Immunomedics, AFP-Cide, developed by Immunomedics; MyelomaCide, developed by Immunomedics; LkoCide, developed by Immunomedics; ProstaCide, developed by Immunomedics; MDX-010, an anti-CTLA4 antibody developed by Medarex; MDX-060, an anti-CD30 antibody developed by Medarex; MDX-070 developed by Medarex; MDX-018 developed by Medarex; OSIDEM (IDM-1), an anti-HER2 antibody developed by Medarex and Immuno-Designed Molecules; HUMAX®-CD4, an anti-CD4 antibody developed by Medarex and Genmab; HuMax-IL15, an anti-IL15 antibody developed by Medarex and Genmab; anti-intercellular adhesion molecule-1 (ICAM-1) (CD54) antibodies developed by MorphoSys, MOR201; tremelimumab, an anti-CTLA-4 antibody developed by Pfizer; visilizumab, an anti-CD3 antibody developed by Protein Design Labs; Anti-a 5β1 Integrin, developed by Protein Design Labs; anti-IL-12, developed by Protein Design Labs; ING-1, an anti-Ep-CAM antibody developed by Xoma; and MLN01, an anti-Beta2 integrin antibody developed by Xoma; all of the above-cited antibody references in this paragraph are expressly incorporated herein by reference. The sequences for the above antibodies can be obtained from publicly available databases, patents, or literature references.

Methods to measure binding affinity and/or other biologic activity of the subject compositions of the invention can be those disclosed herein or methods generally known in the art. For example, the binding affinity of a binding pair (e.g., antibody and antigen), denoted as K_{d}, can be determined using various suitable assays including, but not limited to, radioactive binding assays, non-radioactive binding assays such as fluorescence resonance energy transfer and surface plasmon resonance (SPR, Biacore), and enzyme-linked immunosorbent assays (ELISA), kinetic exclusion assay (KinExA®), reporter gene activity assay, or as described in the Examples. An increase or decrease in binding affinity, for example of a subject therapeutic agent (e.g., a chimeric polypeptide assembly) which has been cleaved to remove a masking moiety compared to the therapeutic agent (e.g., the chimeric polypeptide assembly) with the masking moiety attached, can be determined by measuring the binding affinity of the therapeutic agent (e.g., the chimeric polypeptide assembly) to its target binding partner with and without the masking moiety.

Measurement of half-life of a subject therapeutic agent can be performed by various suitable methods. For example, the half-life of a substance can be determined by administering the substance to a subject and periodically sampling a biological sample (e.g., biological fluid such as blood or plasma or ascites) to determine the concentration and/or amount of that substance in the sample over time. The concentration of a substance in a biological sample can be determined using various suitable methods, including enzyme-linked immunosorbent assays (ELISA), reporter gene activity assays, immunoblots, and chromatography techniques including high-pressure liquid chromatography and fast protein liquid chromatography. In some cases, the substance may be labeled with a detectable tag, such as a radioactive tag or a fluorescence tag, which can be used to determine the concentration of the substance in the sample (e.g., a blood sample, a serum sample, or a plasma sample. The various pharmacokinetic parameters are then determined from the results, which can be done using software packages such as SoftMax Pro software, or by manual calculations known in the art.

In addition, the physicochemical properties of the subject therapeutic agents (e.g., the chimeric polypeptide assembly compositions) may be measured to ascertain the degree of solubility, structure and retention of stability. Assays of the subject compositions are conducted that allow determination of binding characteristics of the binding domains (or binding moieties) towards a ligand, including binding dissociation constant (K_{d}, Kₒₙ and K_{off}), the half-life of dissociation of the ligand-receptor complex, as well as the activity of the binding domain (or binding moiety) to inhibit the biologic activity of the sequestered ligand compared to free ligand (IC₅₀ values). The term “IC₅₀” refers to the concentration needed to inhibit half of the maximum biological response of the ligand agonist, and can be generally determined by competition binding assays. The term “EC₅₀” refers to the concentration needed to achieve half of the maximum biological response of the active substance, and can be generally determined by ELISA or cell-based assays, and/or reporter gene activity assay, including the methods of the Examples described herein.

### Anti-CD3 Binding Domains

The CD3 complex is a group of cell surface molecules that associates with the T-cell antigen receptor (TCR) and functions in the cell surface expression of TCR and in the signaling transduction cascade that originates when a peptide:MHC ligand binds to the TCR. Typically, when an antigen binds to the T-cell receptor, the CD3 sends signals through the cell membrane to the cytoplasm inside the T cell. This causes activation of the T cell that rapidly divide to produce new T cells sensitized to attack the particular antigen to which the TCR were exposed. The CD3 complex is comprised of the CD3epsilon molecule, along with four other membrane-bound polypeptides (CD3-gamma, -delta, -zeta, and -beta). In humans, CD3-epsilon is encoded by the CD3E gene on Chromosome 11. The intracellular domains of each of the CD3 chains contain immunoreceptor tyrosine-based activation motifs (ITAMs) that serve as the nucleating point for the intracellular signal transduction machinery upon T cell receptor engagement.

A number of therapeutic strategies modulate T cell immunity by targeting TCR signalling, particularly the anti-human CD3 monoclonal antibodies (mAbs) that are widely used clinically in immunosuppressive regimes. The CD3-specific mouse mAb OKT3 was the first mAb licensed for use in humans (Sgro, C. Side-effects of a monoclonal antibody, muromonab CD3/orthoclone OKT3: bibliographic review. Toxicology 105:23-29, 1995) and is widely used clinically as an immunosuppressive agent in transplantation (Chatenoud, Clin. Transplant 7:422-430, (1993); Chatenoud, Nat. Rev. Immunol. 3:123-132 (2003); Kumar, Transplant. Proc. 30:1351-1352 (1998)), type 1 diabetes, and psoriasis. Importantly, anti-CD3 mAbs can induce partial T cell signalling and clonal anergy (Smith, JA, Nonmitogenic Anti-CD3 Monoclonal Antibodies Deliver a Partial T Cell Receptor Signal and Induce Clonal Anergy J. Exp. Med. 185:1413-1422 (1997)). OKT3 has been described in the literature as a T cell mitogen as well as a potent T cell killer (Wong, JT. The mechanism of anti-CD3 monoclonal antibodies. Mediation of cytolysis by inter-T cell bridging. Transplantation 50:683-689 (1990)). In particular, the studies of Wong demonstrated that by bridging CD3 T cells and target cells, one could achieve killing of the target and that neither FcR-mediated ADCC nor complement fixation was necessary for bivalent anti-CD3 MAB to lyse the target cells.

OKT3 exhibits both a mitogenic and T-cell killing activity in a time-dependent fashion; following early activation of T cells leading to cytokine release, upon further administration OKT3 later blocks all known T-cell functions. It is due to this later blocking of T cell function that OKT3 has found such wide application as an immunosuppressant in therapy regimens for reduction or even abolition of allograft tissue rejection. Other antibodies specific for the CD3 molecule are disclosed in Tunnacliffe, Int. Immunol. 1 (1989), 546-50, WO2005/118635 and WO2007/033230 describe anti-human monoclonal CD3 epsilon antibodies, U.S. Pat. No. 5,821,337 describes the VL and VH sequences of murine anti-CD3 monoclonal Ab UCHT1 (muxCD3, Shalaby et al., J. Exp. Med. 175, 217-225 (1992) and a humanized variant of this antibody (hu UCHT1), and United States Patent Application 20120034228 discloses binding domains capable of binding to an epitope of human and non-chimpanzee primate CD3 epsilon chain.

TABLE 5a
Anti-CD3 Monoclonal Antibodies and VH & VL Sequences

| Clone | Antibody | | | |
|---|---|---|---|---|
| Name | Name | Target | VH Sequence | VL Sequence |
| huOKT3 | | CD3 | QVQLVQSGGGVVQPGRSLRL | DIQMTQSPSSLSASVGDRVTIT |
| | | | SCKASGYTFTRYTMHWVRQ | CSASSSVSYMNWYQQTPGKA |
| | | | APGKGLEWIGYINPSRGYTN | PKRWIYDTSKLASGVPSRFSG |
| | | | YNQKVKDRFTISRDNSKNTA | SGSGTDYTFTISSLQPEDIATY |
| | | | FLQMDSLRPEDTGVYFCARY | YCQQWSSNPFTFGQGTKLQI |
| | | | YDDHYCLDYWGQGTPVTVS | TR (SEQ ID NO: 479) |
| | | | S (SEQ ID NO: 469) | |
| huUCHT1 | | CD3 | EVQLVESGGGLVQPGGSLRLS | DIQMTQSPSSLSASVGDRVTIT |
| | | | CAASGYSFTGYTMNWVRQA | CRASQDIRNYLNWYQQKPG |
| | | | PGKGLEWVALINPYKGVSTY | KAPKLLIYYTSRLESGVPSRF |
| | | | NQKFKDRFTISVDKSKNTAYL | SGSGSGTDYTLTISSLQPEDFA |
| | | | QMNSLRAEDTAVYYCARSG | TYYCQQGNTLPWTFGQGTK |
| | | | YYGDSDWYFDVWGQGTLVT | VEIK (SEQ ID NO: 480) |
| | | | VSS (SEQ ID NO: 470) | |
| hu12F6 | | CD3 | QVQLVQSGGGVVQPGRSLRL | DIQMTQSPSSLSASVGDRVTM |
| | | | SCKASGYTFTSYTMHWVRQ | TCRASSSVSYMHWYQQTPG |
| | | | APGKGLEWIGYINPSSGYTK | KAPKPWIYATSNLASGVPSRF |
| | | | YNQKFKDRFTISADKSKSTAF | SGSGSGTDYTLTISSLQPEDIA |
| | | | LQMDSLRPEDTGVYFCARW | TYYCQQWSSNPPTFGQGTKL |
| | | | QDYDVYFDYWGQGTPVTVS | QITR (SEQ ID NO: 481) |
| | | | S (SEQ ID NO: 471) | |
| mOKT3 | | CD3 | QVQLQQSGAELARPGASVKM | QIVLTQSPAIMSASPGEKVTM |
| | | | SCKASGYTFTRYTMHWVKQ | TCSASSSVSYMNWYQQKSGT |
| | | | RPGQGLEWIGYINPSRGYTN | SPKRWIYDTSKLASGVPAHF |
| | | | YNQKFKDKATLTTDKSSSTA | RGSGSGTSYSLTISGMEAEDA |
| | | | YMQLSSLTSEDSAVYYCARY | ATYYCQQWSSNPFTFGSGTK |
| | | | YDDHYCLDYWGQGTTLTVS | LEINR (SEQ ID NO: 482) |
| | | | S (SEQ ID NO: 472) | |
| MT103 | blinatumo | CD3 | DIKLQQSGAELARPGASVKM | DIQLTQSPAIMSASPGEKVTM |
| | mab | | SCKTSGYTFTRYTMHWVKQ | TCRASSSVSYMNWYQQKSGT |
| | | | RPGQGLEWIGYINPSRGYTN | SPKRWIYDTSKVASGVPYRFS |
| | | | YNQKFKDKATLTTDKSSSTA | GSGSGTSYSLTISSMEAEDAA |
| | | | YMQLSSLTSEDSAVYYCARY | TYYCQQWSSNPLTFGAGTKL |
| | | | YDDHYCLDYWGQGTTLTVS | ELK (SEQ ID NO: 483) |
| | | | S (SEQ ID NO: 473) | |
| MT110 | solitomab | CD3 | DVQLVQSGAEVKKPGASVKV | DIVLTQSPATLSLSPGERATLS |
| | | | SCKASGYTFTRYTMHWVRQ | CRASQSVSYMNWYQQKPGK |
| | | | APGQGLEWIGYINPSRGYTN | APKRWIYDTSKVASGVPARF |
| | | | YADSVKGRFTITTDKSTSTAY | SGSGSGTDYSLTINSLEAEDA |
| | | | MELSSLRSEDTATYYCARYY | ATYYCQQWSSNPLTFGGGT |
| | | | DDHYCLDYWGQGTTVTVSS | KVEIK (SEQ ID NO: 484) |
| | | | (SEQ ID NO: 474) | |
| CD3.7 | | CD3 | EVQLVESGGGLVQPGGSLKL | QTVVTQEPSLTVSPGGTVTLT |
| | | | SCAASGFTFNKYAMNWVRQ | CGSSTGAVTSGYYPNWVQQK |
| | | | APGKGLEWVARIRSKYNNYA | PGQAPRGLIGGTKFLAPGTPA |
| | | | TYYADSVKDRFTISRDDSKNT | RFSGSLLGGKAALTLSGVQPE |
| | | | AYLQMNNLKTEDTAVYYCV | DEAEYYCALWYSNRWVFGG |
| | | | RHGNFGNSYISYWAYWGQG | GTKLTVL (SEQ ID NO: 485) |
| | | | TLVTVSS (SEQ ID NO: 475) | |
| CD3.8 | | CD3 | EVQLVESGGGLVQPGGSLRLS | QAVVTQEPSLTVSPGGTVTLT |
| | | | CAASGFTFNTYAMNWVRQA | CGSSTGAVTTSNYANWVQQK |
| | | | PGKGLEWVGRIRSKYNNYAT | PGQAPRGLIGGTNKRAPGVPA |
| | | | YYADSVKGRFTISRDDSKNTL | RFSGSLLGGKAALTLSGAQPE |
| | | | YLQMNSLRAEDTAVYYCVR | DEAEYYCALWYSNLWVFGG |
| | | | HGNFGNSYVSWFAYWGQGT | GTKLTVL (SEQ ID NO: 486) |
| | | | LVTVSS (SEQ ID NO: 476) | |
| CD3.9 | | CD3 | EVQLLESGGGLVQPGGSLKLS | ELVVTQEPSLTVSPGGTVTLT |
| | | | CAASGFTFNTYAMNWVRQA | CRSSTGAVTTSNYANWVQQK |
| | | | PGKGLEWVARIRSKYNNYAT | PGQAPRGLIGGTNKRAPGTPA |
| | | | YYADSVKDRFTISRDDSKNTA | RFSGSLLGGKAALTLSGVQPE |
| | | | YLQMNNLKTEDTAVYYCVR | DEAEYYCALWYSNLWVFGG |
| | | | HGNFGNSYVSWFAYWGQGT | GTKLTVL (SEQ ID NO: 487) |
| | | | LVTVSS (SEQ ID NO: 477) | |
| CD3.10 | | CD3 | EVKLLESGGGLVQPKGSLKLS | QAVVTQESALTTSPGETVTLT |
| | | | CAASGFTFNTYAMNWVRQA | CRSSTGAVTTSNYANWVQEK |
| | | | PGKGLEWVARIRSKYNNYAT | PDHLFTGLIGGTNKRAPGVPA |
| | | | YYADSVKDRFTISRDDSQSIL | RFSGSLIGDKAALTITGAQTE |
| | | | YLQMNNLKTEDTAMYYCVR | DEAIYFCALWYSNLWVFGGG |
| | | | HGNFGNSYVSWFAYWGQGT | TKLTVL (SEQ ID NO: 488) |
| | | | LVTVSS (SEQ ID NO: 478) | |

*underlined sequences, if present, are CDRs within the VL and VH

In some embodiments of the compositions of this disclosure, the BP can comprise a binding domain (or a binding moiety) (such as an antigen binding fragment) having specific binding affinity for an effector cell antigen. The effector cell antigen can be expressed on the surface of an effector cell selected from a plasma cell, a T cell, a B cell, a cytokine induced killer cell (CIK cell), a mast cell, a dendritic cell, a regulatory T cell (RegT cell), a helper T cell, a myeloid cell, and a NK cell. The effector cell antigen can be expressed on the surface of a T cell. The binding domain (or binding moiety) can have binding affinity for CD3. In some embodiments, where the binding domain (or binding moiety) having binding affinity for CD3, the binding domain (or binding moiety) can have binding affinity for a member of the CD3 complex, which includes in individual form or independently combined form all known CD3 subunits of the CD3 complex; for example, CD3 epsilon, CD3 delta, CD3 gamma, CD3 zeta, CD3 alpha and CD3 beta. The binding domain (or binding moiety) having binding affinity for CD3 can have binding affinity for CD3 epsilon, CD3 delta, CD3 gamma, CD3 zeta, CD3 alpha or CD3 beta.

The origin of the antigen binding fragments (comprised in the binding domain or binding moiety) contemplated by the disclosure can be derived from a naturally occurring antibody or fragment thereof, a non-naturally occurring antibody or fragment thereof, a humanized antibody or fragment thereof, a synthetic antibody or fragment thereof, a hybrid antibody or fragment thereof, or an engineered antibody or fragment thereof. Methods for generating an antibody for a given target marker are well known in the art. For example, the monoclonal antibodies may be made using the hybridoma method first described by Kohler et al., Nature, 256:495 (1975), or may be made by recombinant DNA methods (U.S. Pat. No. 4,816,567). The structure of antibodies and fragments thereof, variable regions of heavy and light chains of an antibody (VH and VL), single chain variable regions (scFv), complementarity determining regions (CDR), and domain antibodies (dAbs) are well understood. Methods for generating a polypeptide having a desired antigen binding fragment with binding affinity to a given antigen are known in the art.

It will be understood that use of the term antigen binding fragments for the composition embodiments disclosed herein is intended to include portions or fragments of antibodies that retain the ability to bind the antigens that are the ligands of the corresponding intact antibody. In such embodiments, the antigen binding fragment can be, but is not limited to, CDRs and intervening framework regions, variable or hypervariable regions of light and/or heavy chains of an antibody (VL, VH), variable fragments (Fv), Fab′ fragments, F(ab′)2 fragments, Fab fragments, single chain antibodies (scAb), VHH camelid antibodies, single chain variable fragment (scFv), linear antibodies, a single domain antibody, complementarity determining regions (CDR), domain antibodies (dAbs), single domain heavy chain immunoglobulins of the BHH or BNAR type, single domain light chain immunoglobulins, or other polypeptides known in the art containing a fragment of an antibody capable of binding an antigen. The antigen binding fragments having CDR-H and CDR-L can be configured in a (CDR-H)-(CDR-L) or a (CDR-H)-(CDR-L) orientation, N-terminus to C-terminus. The VL and VH of two antigen binding fragments can also be configured in a single chain diabody configuration; e.g., the VL and VH of the first and second binding domains (or binding moieties) configured with linkers of an appropriate length to permit arrangement as a diabody.

Various CD3 binding domains of the disclosure have been specifically modified to enhance their stability in the polypeptide embodiments described herein. Binding specificity can be determined by complementarity determining regions (CDRs), such as light chain CDRs or heavy chain CDRs. In many cases, binding specificity is determined by light chain CDRs and heavy chain CDRs. A given combination of heavy chain CDRs and light chain CDRs provides a given binding pocket that confers greater affinity and/or specificity towards an effector cell antigen as compared to other reference antigens. Protein aggregation of antibodies continues to be a significant problem in their developability and remains a major area of focus in antibody production. Antibody aggregation can be triggered by partial unfolding of its domains, leading to monomer-monomer association followed by nucleation and aggregate growth. Although the aggregation propensities of antibodies and antibody-based proteins can be affected by the external experimental conditions, they are strongly dependent on the intrinsic antibody properties as determined by their sequences and structures. Although it is well known that proteins are only marginally stable in their folded states, it is often less well appreciated that most proteins are inherently aggregation-prone in their unfolded or partially unfolded states, and the resulting aggregates can be extremely stable and long-lived. Reduction in aggregation propensity has also been shown to be accompanied by an increase in expression titer, showing that reducing protein aggregation is beneficial throughout the development process and can lead to a more efficient path to clinical studies. For therapeutic proteins, aggregates are a significant risk factor for deleterious immune responses in patients, and can form via a variety of mechanisms. Controlling aggregation can improve protein stability, manufacturability, attrition rates, safety, formulation, titers, immunogenicity, and solubility. The intrinsic properties of proteins such as size, hydrophobicity, electrostatics and charge distribution play important roles in protein solubility. Low solubility of therapeutic proteins due to surface hydrophobicity has been shown to render formulation development more difficult and may lead to poor bio-distribution, undesirable pharmacokinetics behavior and immunogenicity in vivo. Decreasing the overall surface hydrophobicity of candidate monoclonal antibodies can also provide benefits and cost savings relating to purification and dosing regimens. Individual amino acids can be identified by structural analysis as being contributory to aggregation potential in an antibody, and can be located in CDR as well as framework regions. In particular, residues can be predicted to be at high risk of causing hydrophobicity issues in a given antibody.

In some embodiments, the invention provides therapeutic agents that comprise binding domain(s) with binding affinity to T cell antigen(s). In some embodiments, the binding domain with binding affinity to a T cell antigen can comprise VL and VH derived from a monoclonal antibody to an antigen of the cluster of differentiation 3 T cell receptor (CD3). The binding domain can comprise VL and VH derived from a monoclonal antibody to CD3epsilon and CD3delta subunits. Monoclonal antibodies to CD3 neu are known in the art. Exemplary, non-limiting examples of VL and VH sequences of monoclonal antibodies to CD3 are presented in Table 5a. The binding domain with binding affinity to CD3 can comprise anti-CD3 VL and VH sequences set forth in Table 5a. The binding domain with binding affinity to CD3epsilon can comprise anti-CD3epsilon VL and VH sequences set forth in Table 5a. The binding domain with binding affinity to CD3 can comprise VH and VL regions wherein each VH and VL regions exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99%, or 100% identity to paired VL and VH sequences of the huUCHT1 anti-CD3 antibody of Table 5a. The binding domain with binding affinity to CD3 can comprise the CDR-L1 region, the CDR-L2 region, the CDR-L3 region, the CDR-H1 region, the CDR-H2 region, and the CDR-H3 region, wherein each is derived from the respective anti-CD3 VL and VH sequences set forth in Table 5a. The binding domain with binding affinity to CD3 can comprise the CDR-L1 region, the CDR-L2 region, the CDR-L3 region, the CDR-H1 region, the CDR-H2 region, and the CDR-H3 region, wherein the CDR sequences. The binding domain with binding affinity to CD3 can comprise the CDR-L1 region, the CDR-L2 region, the CDR-L3 region, the CDR-H1 region, the CDR-H2 region, and the CDR-H3 region, wherein the CDR sequences are RASQDIRNYLN (SEQ ID NO: 489), YTSRLES (SEQ ID NO: 490), QQGNTLPWT (SEQ ID NO: 491), GYSFTGYTMN (SEQ ID NO: 492), LINPYKGVST (SEQ ID NO: 493), and SGYYGDSDWYFDV (SEQ ID NO: 494).

In some embodiments, the present disclosure provides a binding domain (or binding moiety) that binds CD3, for incorporation into the compositions described herein, can comprise CDR-L and CDR-H. The binding domain binding CD3 can comprise a CDR-H1, a CDR-H2, and a CDR-H3, each (independently) having an amino acid sequence exhibiting at least 85%, 86%, 87%, 88%, 89%, 90%, 91%, 92%, 93%, 94%, 95%, 96%, 97%, 98%, 99% sequence identity or is identical to the amino acid sequence set forth in Table 5b. The binding domain binding CD3 can comprise a CDR-L1, a CDR-L2, and a CDR-L3, each (independently) having an amino acid sequence exhibiting at least 85%, 86%, 87%, 88%, 89%, 90%, 91%, 92%, 93%, 94%, 95%, 96%, 97%, 98%, 99% sequence identity or is identical to the amino acid sequence set forth in Table 5b.

In some embodiments, the present disclosure provides a binding domain (or binding moiety) that binds CD3, for incorporation into the compositions described herein, can comprise light chain framework regions (FR-L) and heavy chain framework regions (FR-H). The binding domain binding CD3 can comprise a FR-L1 exhibiting at least 86%, 87%, 88%, 89%, 90%, 91%, 92%, 93%, 94%, 95%, 96%, 97%, 98%, 99% sequence identity or is identical to a FR-L1 sequence set forth in Table 5c. The binding domain binding CD3 can comprise a FR-L2 exhibiting at least 86%, 87%, 88%, 89%, 90%, 91%, 92%, 93%, 94%, 95%, 96%, 97%, 98%, 99% sequence identity or is identical to a FR-L2 sequence set forth in Table 5c. The binding domain binding CD3 can comprise a FR-L3 exhibiting at least 86%, 87%, 88%, 89%, 90%, 91%, 92%, 93%, 94%, 95%, 96%, 97%, 98%, 99% sequence identity or is identical to a FR-L3 sequence set forth in Table 5c. The binding domain binding CD3 can comprise a FR-L4 exhibiting at least 86%, 87%, 88%, 89%, 90%, 91%, 92%, 93%, 94%, 95%, 96%, 97%, 98%, 99% sequence identity or is identical to a FR-L4 sequence set forth in Table 5c. The binding domain binding CD3 can comprise a FR-H1 exhibiting at least 86%, 87%, 88%, 89%, 90%, 91%, 92%, 93%, 94%, 95%, 96%, 97%, 98%, 99% sequence identity or is identical to a FR-H1 sequence set forth in Table 5c. The binding domain binding CD3 can comprise a FR-H2 exhibiting at least 86%, 87%, 88%, 89%, 90%, 91%, 92%, 93%, 94%, 95%, 96%, 97%, 98%, 99% sequence identity or is identical to a FR-H2 sequence set forth in Table 5c. The binding domain binding CD3 can comprise a FR-H3 exhibiting at least 86%, 87%, 88%, 89%, 90%, 91%, 92%, 93%, 94%, 95%, 96%, 97%, 98%, 99% sequence identity or is identical to a FR-H3 sequence set forth in Table 5c. The binding domain binding CD3 can comprise a FR-H4 exhibiting at least 86%, 87%, 88%, 89%, 90%, 91%, 92%, 93%, 94%, 95%, 96%, 97%, 98%, 99% sequence identity or is identical to a FR-H4 sequence set forth in Table 5c.

In some embodiments, the present disclosure provides a binding domain (or binding moiety) that binds CD3, for incorporation into the compositions described herein, can comprise a variable light (VL) amino acid sequence and a variable heavy (VH) amino acid sequence. The binding domain that binds CD3 can comprise a VL exhibiting at least 86%, 87%, 88%, 89%, 90%, 91%, 92%, 93%, 94%, 95%, 96%, 97%, 98%, 99% sequence identity or is identical to a VL sequence set forth in Table 5d. The binding domain that binds CD3 can comprise a VH exhibiting at least 86%, 87%, 88%, 89%, 90%, 91%, 92%, 93%, 94%, 95%, 96%, 97%, 98%, 99% sequence identity or is identical to a VH sequence set forth in Table 5d. The binding domain that binds CD3 can comprise an amino acid sequence exhibiting at least 86%, 87%, 88%, 89%, 90%, 91%, 92%, 93%, 94%, 95%, 96%, 97%, 98%, 99% sequence identity or is identical to a scFv sequence set forth in Table 5d.

In some embodiments of the compositions of this disclosure, the VL and VH of the antigen binding fragments can be fused by relatively long linkers, consisting 25, 26, 27, 28, 29, 30, 31, 32, 33, 34, or 35 hydrophilic amino acids that, when joined together, have a flexible characteristic. In some embodiment, the VL and VH of any of the scFv embodiments described herein can be linked by relatively long linkers of hydrophilic amino acids selected from the sequences GSGEGSEGEGGGEGSEGEGSGEGGEGEGSG (SEQ ID NO: 495), TGSGEGSEGEGGGEGSEGEGSGEGGEGEGSGT (SEQ ID NO: 496), GATPPETGAETESPGETTGGSAESEPPGEG (SEQ ID NO: 497), or GSAAPTAGTTPSASPAPPTGGSSAAGSPST (SEQ ID NO: 498).

In some embodiments of the compositions of this disclosure, where the BP comprises a first binding domain (or first binding moiety) and a second binding domain (or second binding moiety), the first and second binding domains (or the first and second binding moieties) can be linked together by a short linker of hydrophilic amino acids having 3, 4, 5, 6, or 7 amino acids. The short linker sequences can be selected from the group of sequences SGGGGS (SEQ ID NO: 499), GGGGS (SEQ ID NO: 500), GGSGGS (SEQ ID NO: 501), GGS, or GSP. In some embodiment, the disclosure provides compositions comprising a single chain diabody in which after folding, the first domain (VL or VH) is paired with the last domain (VH or VL) to form one scFv and the two domains in the middle are paired to form the other scFv in which the first and second domains, as well as the third and last domains, are fused together by one of the foregoing short linkers and the second and the third variable domains are fused by one of the foregoing relatively long linkers. As will be appreciated by one of skill in the art, the selection of the short linker and relatively long linker can be to prevent the incorrect pairing of adjacent variable domains, thereby facilitating the formation of the single chain diabody configuration comprising the VL and VH of the first antigen binding fragment and the second antigen binding fragment.

TABLE 5b
Exemplary CD3 CDR Sequences

| | | SEQ ID | |
|---|---|---|---|
| Construct | Region | NO: | Amino Acid Sequence |
| 3.23, 3.30, 3.31, 3.32 | CDR-L1 | 502 | RSSNGAVTSSNYAN |
| 3.24 | CDR-L1 | 503 | RSSNGEVTTSNYAN |
| 3.33, 3.9 | CDR-L1 | 504 | RSSTGAVTTSNYAN |
| 3.23, 3.30, 3.31, 3.32, 3.9, 3.33 | CDR-L2 | 505 | GTNKRAP |
| 3.24 | CDR-L2 | 506 | GTIKRAP |
| 3.23, 3.24, 3.30, 3.31, 3.32 | CDR-L3 | 507 | ALWYPNLWVF |
| 3.33, 3.9 | CDR-L3 | 508 | ALWYSNLWVF |
| 3.23, 3.24, 3.30, 3.31, 3.32, | CDR-H1 | 509 | GFTFNTYAMN |
| 3.9, 3.33 | | | |
| 3.23, 3.24, 3.30, 3.31, 3.32, | CDR-H2 | 510 | RIRSKYNNYATYYADSVKD |
| 3.9, 3.33 | | | |
| 3.23. 3.24, 3.30, 3.31, 3.32 | CDR-H3 | 511 | HENFGNSYVSWFAH |
| 3.9, 3.33 | CDR-H3 | 512 | HGNFGNSYVSWFAY |

TABLE 5c
Exemplary CD3 FR Sequences

| | | SEQ | |
|---|---|---|---|
| Construct | Region | ID NO: | Amino Acid Sequence |
| 3.23, 3.24, 3.30, | FR-L1 | 513 | ELVVTQEPSLTVSPGGTVTLTC |
| 3.31, 3.32, 3.9, | | | |
| 3.33 | | | |
| 3.23, 3.24, 3.30, | FR-L2 | 514 | WVQQKPGQAPRGLIG |
| 3.31, 3.32, 3.9, | | | |
| 3.33 | | | |
| 3.23, 3.24 | FR-L3 | 515 | GTPARFSGSLLGGKAALTLSGVQPEDEAVYYC |
| 3.30 | FR-L3 | 516 | GTPARFSGSSLGGKAALTLSGVQPEDEAVYYC |
| 3.31 | FR-L3 | 517 | GTPARFSGSLLGGSAALTLSGVQPEDEAVYYC |
| 3.32 | FR-L3 | 518 | GTPARFSGSSLGGSAALTLSGVQPEDEAVYYC |
| 3.9 | FR-L3 | 519 | GTPARFSGSLLGGKAALTLSGVQPEDEAEYYC |
| 3.33 | FR-L3 | 520 | GTPARFSGSSLGGSAALTLSGVQPEDEAEYYC |
| 3.23, 3.24, 3.30, | FR-L4 | 521 | GGGTKLTVL |
| 3.31, 3.32, 3.9, | | | |
| 3.33 | | | |
| 3.23. 3.24 | FR-H1 | 522 | EVQLLESGGGIVQPGGSLKLSCAAS |
| 3.30, 3.31, 3.32 | FR-H1 | 523 | EVQLQESGGGIVQPGGSLKLSCAAS |
| 3.33 | FR-H1 | 524 | EVQLQESGGGLVQPGGSLKLSCAAS |
| 3.9 | FR-H1 | 525 | EVQLLESGGGLVQPGGSLKLSCAAS |
| 3.23, 3.24, 3.30, | FR-H2 | 526 | WVRQAPGKGLEWVA |
| 3.31, 3.32, 3.9, | | | |
| 3.33 | | | |
| 3.23, 3.24, 3.30, | FR-H3 | 527 | RFTISRDDSKNTVYLQMNNLKTEDTAVYYCVR |
| 3.31, 3.32 | | | |
| 3.9. 3.33 | FR-H3 | 528 | RFTISRDDSKNTAYLQMNNLKTEDTAVYYCVR |
| 3.23, 3.24, 3.30, | FR-H4 | 529 | WGQGTLVTVSS |
| 3.31, 3.32, 3.9, | | | |
| 3.33 | | | |

TABLE 5d
Exemplary VL & VH Sequences

| | | SEQ ID | |
|---|---|---|---|
| Construct | Region | NO: | Amino Acid Sequence |
| 3.23 | VL | 530 | ELVVTQEPSLTVSPGGTVTLTCRSSNGAVTSSNYANWVQQKPGQAPR |
| | | | GLIGGTNKRAPGTPARFSGSLLGGKAALTLSGVQPEDEAVYYCALWY |
| | | | PNLWVFGGGTKLTVL |
| 3.23, | VH | 531 | EVQLLESGGGIVQPGGSLKLSCAASGFTFNTYAMNWVRQAPGKGLEW |
| 3.24 | | | VARIRSKYNNYATYYADSVKDRFTISRDDSKNTVYLQMNNLKTEDTA |
| | | | VYYCVRHENFGNSYVSWFAHWGQGTLVTVSS |
| 3.24 | VL | 532 | ELVVTQEPSLTVSPGGTVTLTCRSSNGEVTTSNYANWVQQKPGQAPR |
| | | | GLIGGTIKRAPGTPARFSGSLLGGKAALTLSGVQPEDEAVYYCALWYP |
| | | | NLWVFGGGTKLTVL |
| 3.30 | VL | 533 | ELVVTQEPSLTVSPGGTVTLTCRSSNGAVTSSNYANWVQQKPGQAPR |
| | | | GLIGGTNKRAPGTPARFSGSSLGGKAALTLSGVQPEDEAVYYCALWY |
| | | | PNLWVFGGGTKLTVL |
| 3.30, | VH | 534 | EVQLQESGGGIVQPGGSLKLSCAASGFTFNTYAMNWVRQAPGKGLEW |
| 3.31, | | | VARIRSKYNNYATYYADSVKDRFTISRDDSKNTVYLQMNNLKTEDTA |
| 3.32 | | | VYYCVRHENFGNSYVSWFAHWGQGTLVTVSS |
| 3.31 | VL | 535 | ELVVTQEPSLTVSPGGTVTLTCRSSNGAVTSSNYANWVQQKPGQAPR |
| | | | GLIGGTNKRAPGTPARFSGSLLGGSAALTLSGVQPEDEAVYYCALWYP |
| | | | NLWVFGGGTKLTVL |
| 3.32 | VL | 536 | ELVVTQEPSLTVSPGGTVTLTCRSSNGAVTSSNYANWVQQKPGQAPR |
| | | | GLIGGTNKRAPGTPARFSGSSLGGSAALTLSGVQPEDEAVYYCALWYP |
| | | | NLWVFGGGTKLTVL |
| 3.9 | VL | 537 | ELVVTQEPSLTVSPGGTVTLTCRSSTGAVTTSNYANWVQQKPGQAPR |
| | | | GLIGGTNKRAPGTPARFSGSLLGGKAALTLSGVQPEDEAEYYCALWYS |
| | | | NLWVFGGGTKLTVL |
| 3.9 | VH | 538 | EVQLLESGGGLVQPGGSLKLSCAASGFTFNTYAMNWVRQAPGKGLE |
| | | | WVARIRSKYNNYATYYADSVKDRFTISRDDSKNTAYLQMNNLKTEDT |
| | | | AVYYCVRHGNFGNSYVSWFAYWGQGTLVTVSS |
| 3.33 | VL | 539 | ELVVTQEPSLTVSPGGTVTLTCRSSTGAVTTSNYANWVQQKPGQAPR |
| | | | GLIGGTNKRAPGTPARFSGSSLGGSAALTLSGVQPEDEAEYYCALWYS |
| | | | NLWVFGGGTKLTVL |
| 3.33 | VH | 540 | EVQLQESGGGLVQPGGSLKLSCAASGFTFNTYAMNWVRQAPGKGLE |
| | | | WVARIRSKYNNYATYYADSVKDRFTISRDDSKNTAYLQMNNLKTEDT |
| | | | AVYYCVRHGNFGNSYVSWFAYWGQGTLVTVSS |

TABLE 5e
Exemplary scFv Sequences

| | SEQ ID | |
|---|---|---|
| Construct | NO: | Amino Acid Sequence |
| 3.23 | 541 | ELVVTQEPSLTVSPGGTVTLTCRSSNGAVTSSNYANWVQQKPGQAPRGLIGG |
| | | TNKRAPGTPARFSGSLLGGKAALTLSGVQPEDEAVYYCALWYPNLWVFGG |
| | | GTKLTVLGATPPETGAETESPGETTGGSAESEPPGEGEVQLLESGGGIVQPGG |
| | | SLKLSCAASGFTFNTYAMNWVRQAPGKGLEWVARIRSKYNNYATYYADSV |
| | | KDRFTISRDDSKNTVYLQMNNLKTEDTAVYYCVRHENFGNSYVSWFAHWG |
| | | QGTLVTVSS |
| 3.24 | 542 | ELVVTQEPSLTVSPGGTVTLTCRSSNGEVTTSNYANWVQQKPGQAPRGLIGG |
| | | TIKRAPGTPARFSGSLLGGKAALTLSGVQPEDEAVYYCALWYPNLWVFGGG |
| | | TKLTVLGATPPETGAETESPGETTGGSAESEPPGEGEVQLLESGGGIVQPGGS |
| | | LKLSCAASGFTFNTYAMNWVRQAPGKGLEWVARIRSKYNNYATYYADSVK |
| | | DRFTISRDDSKNTVYLQMNNLKTEDTAVYYCVRHENFGNSYVSWFAHWGQ |
| | | GTLVTVSS |
| 3.30 | 543 | ELVVTQEPSLTVSPGGTVTLTCRSSNGAVTSSNYANWVQQKPGQAPRGLIGG |
| | | TNKRAPGTPARFSGSSLGGKAALTLSGVQPEDEAVYYCALWYPNLWVFGGG |
| | | TKLTVLGATPPETGAETESPGETTGGSAESEPPGEGEVQLQESGGGIVQPGGS |
| | | LKLSCAASGFTFNTYAMNWVRQAPGKGLEWVARIRSKYNNYATYYADSVK |
| | | DRFTISRDDSKNTVYLQMNNLKTEDTAVYYCVRHENFGNSYVSWFAHWGQ |
| | | GTLVTVSS |
| 3.31 | 544 | ELVVTQEPSLTVSPGGTVTLTCRSSNGAVTSSNYANWVQQKPGQAPRGLIGG |
| | | TNKRAPGTPARFSGSLLGGSAALTLSGVQPEDEAVYYCALWYPNLWVFGGG |
| | | TKLTVLGATPPETGAETESPGETTGGSAESEPPGEGEVQLQESGGGIVQPGGS |
| | | LKLSCAASGFTFNTYAMNWVRQAPGKGLEWVARIRSKYNNYATYYADSVK |
| | | DRFTISRDDSKNTVYLQMNNLKTEDTAVYYCVRHENFGNSYVSWFAHWGQ |
| | | GTLVTVSS |
| 3.32 | 545 | ELVVTQEPSLTVSPGGTVTLTCRSSNGAVTSSNYANWVQQKPGQAPRGLIGG |
| | | TNKRAPGTPARFSGSSLGGSAALTLSGVQPEDEAVYYCALWYPNLWVFGGG |
| | | TKLTVLGATPPETGAETESPGETTGGSAESEPPGEGEVQLQESGGGIVQPGGS |
| | | LKLSCAASGFTFNTYAMNWVRQAPGKGLEWVARIRSKYNNYATYYADSVK |
| | | DRFTISRDDSKNTVYLQMNNLKTEDTAVYYCVRHENFGNSYVSWFAHWGQ |
| | | GTLVTVSS |
| 3.9 | 546 | ELVVTQEPSLTVSPGGTVTLTCRSSTGAVTTSNYANWVQQKPGQAPRGLIGG |
| | | TNKRAPGTPARFSGSLLGGKAALTLSGVQPEDEAEYYCALWYSNLWVFGGG |
| | | TKLTVLGATPPETGAETESPGETTGGSAESEPPGEGEVQLLESGGGLVQPGGS |
| | | LKLSCAASGFTFNTYAMNWVRQAPGKGLEWVARIRSKYNNYATYYADSVK |
| | | DRFTISRDDSKNTAYLQMNNLKTEDTAVYYCVRHGNFGNSYVSWFAYWGQ |
| | | GTLVTVSS |
| 3.33 | 547 | ELVVTQEPSLTVSPGGTVTLTCRSSTGAVTTSNYANWVQQKPGQAPRGLIGG |
| | | TNKRAPGTPARFSGSSLGGSAALTLSGVQPEDEAEYYCALWYSNLWVFGGG |
| | | TKLTVLGATPPETGAETESPGETTGGSAESEPPGEGEVQLQESGGGLVQPGGS |
| | | LKLSCAASGFTFNTYAMNWVRQAPGKGLEWVARIRSKYNNYATYYADSVK |
| | | DRFTISRDDSKNTAYLQMNNLKTEDTAVYYCVRHGNFGNSYVSWFAYWGQ |
| | | GTLVTVSS |
| 4.11 | 548 | QSVLTQPPSASGTPGQRVTISCSGSSSNIGSNYVYWYQQLPGTAPKLLIYRNN |
| | | QRPSGVPDRFSGSKSGTSASLAISGLRSEDEADYYCAAWDDSLSGLWVFGGG |
| | | TKLTVLGATPPETGAETESPGETTGGSAESEPPGEGQVQLQQWGGGLVKPGG |
| | | SLRLSCAASGFTFSSYSMNWVRQAPGKGLEWVSRINSDGSSTNYADSVKGRF |
| | | TISRDNAKNTLYLQMNSLRAEDTAVYYCARELRWGNWGQGTLVTVSS |
| 4.12 | 549 | QAGLTQPPSASGTPGQRVTLSCSGSYSNIGTYYVYWYQQLPGTAPKLLIYSN |
| | | DQRLSGVPDRFSGSKSGTSASLAISGLQSEDEAAYYCAAWDDSLNGWAFGG |
| | | GTKLTVLGATPPETGAETESPGETTGGSAESEPPGEGQVQLQQWGGGLVKPG |
| | | GSLRLSCAASGFTFSSYSMNWVRQAPGKGLEWVSRINSDGSSTNYADSVKG |
| | | RFTISRDNAKNTLYLQMNSLRAEDTAVYYCARELRWGNWGQGTLVTVSS |
| 4.13 | 550 | QPGLTQPPSASGTPGQRVTLSCSGRSSNIGSYYVYWYQHLPGMAPKLLIYRN |
| | | SRRPSGVPDRFSGSKSGTSASLVISGLQSDDEADYYCAAWDDSLKSWVFGGG |
| | | TKLTVLGATPPETGAETESPGETTGGSAESEPPGEGQVQLQQWGGGLVKPGG |
| | | SLRLSCAASGFTFSSYSMNWVRQAPGKGLEWVSRINSDGSSTNYADSVKGRF |
| | | TISRDNAKNTLYLQMNSLRAEDTAVYYCARELRWGNWGQGTLVTVSS |
| 4.14 | 551 | QSVLTQPPSASGTPGQRVTISCSGSSSNIGTNYVYWYQQFPGTAPKLLIYSNN |
| | | QRPSGVPDRFSGSKSGTSGSLAISGLQSEDEADYSCAAWDDSLNGWVFGGGT |
| | | KLTVLGATPPETGAETESPGETTGGSAESEPPGEGQVQLVQWGGGLVKPGGS |
| | | LRLSCAASGFTFSSYSMNWVRQAPGKGLEWVSRINSDGSSTNYADSVKGRF |
| | | TISRDNAKNTLYLQMNSLRAEDTAVYYCARELRWGNWGQGTLVTVSS |
| 4.15 | 552 | QPGLTQPPSASGTPGQRVTISCSGSSSNIGSNYVYWYQQLPGTAPKLLIYRNN |
| | | QRPSGVPDRLSGSKSGTSASLAISGLRSEDEADYYCAAWDDSLSGWVFGGGT |
| | | KLTVLGATPPETGAETESPGETTGGSAESEPPGEGQVQLVQWGGGLVKPGGS |
| | | LRLSCAASGFTFSSYSMNWVRQAPGKGLEWVSRINSDGSSTNYADSVKGRF |
| | | TISRDNAKNTLYLQMNSLRAEDTAVYYCARELRWGNWGQGTLVTVSS |
| 4.16 | 553 | QAVLTQPPSASGTPGQRVTISCSGSSSNIGSYYVYWYQQVPGAAPKLLMRLN |
| | | NQRPSGVPDRFSGAKSGTSASLVISGLRSEDEADYYCAAWDDSLSGQWVFG |
| | | GGTKLTVLGATPPETGAETESPGETTGGSAESEPPGEGQVQLQQWGGGLVKP |
| | | GGSLRLSCAASGFTFSSYSMNWVRQAPGKGLEWVSRINSDGSSTNYADSVK |
| | | GRFTISRDNAKNTLYLQMNSLRAEDTAVYYCARELRWGNWGQGTLVTVSS |
| 4.17 | 554 | QAGLTQPPSASGTPGQRVTISCSGSSSNIGSNYVYWYQQLPGTAPKLLIYRNN |
| | | QRPSGVPDRFSGSKSGTSASLAISGLRSEDEADYYCATWDASLSGWVFGGGT |
| | | KLTVLGATPPETGAETESPGETTGGSAESEPPGEGEVQLVQWGGGLVKPGGS |
| | | LRLSCAASGFTFSSYSMNWVRQAPGKGLEWVSRINSDGSSTNYADSVKGRF |
| | | TISRDNAKNTLYLQMNSLRAEDTAVYYCARELRWGNWGQGTLVTVSS |

### Tumor-Specific Markers or Antigens of Target Cells

In some embodiments of the compositions of this disclosure, the binding domain (e.g., the first binding domain) can have specific binding affinity to a tumor-specific marker or an antigen of a target cell. Some embodiments of the compositions of this disclosure can comprise another binding domain (e.g., the second binding domain) that binds to an effector cell antigen. The tumor-specific marker can be associated with a tumor cell (such as of stroma cell tumor, fibroblast tumor, myofibroblast tumor, glial cell tumor, epithelial cell tumor, fat cell tumor, immune cell tumor, vascular cell tumor, or smooth muscle cell tumor). The tumor-specific marker or the antigen of the target cell can be selected from the group consisting of alpha 4 integrin, Ang2, B7-H3, B7-H6 (e.g., its natural ligand Nkp30 rather than an antibody fragment), CEACAM5, cMET, CTLA4, FOLR1, EpCAM (epithelial cell adhesion molecule), CCR5, CD19, HER2, HER2 neu, HER3, HER4, HER1 (EGFR), PD-L1, PSMA, CEA, TROP-2, MUC1(mucin), MUC-2, MUC3, MUC4, MUC5AC, MUC5B, MUC7, MUC16, βhCG, Lewis-Y, CD20, CD33, CD38, CD30, CD56 (NCAM), CD133, ganglioside GD3, 9-O-acetyl-GD3, GM2, Globo H, fucosyl GM1, GD2, carbonicanhydrase IX, CD44v6, Nectin-4, Sonic Hedgehog (Shh), Wue-1, plasma cell antigen 1 (PC-1), melanoma chondroitin sulfate proteoglycan (MCSP), CCR8, 6-transmembrane epithelial antigen of prostate (STEAP), mesothelin, A33 antigen, prostate stem cell antigen (PSCA), Ly-6, desmoglein 4, fetal acetylcholine receptor (fnAChR), CD25, cancer antigen 19-9 (CA19-9), cancer antigen 125 (CA-125), Muellerian inhibitory substance receptor type II (MISIIR), sialylated Tn antigen (sTN), fibroblast activation antigen (FAP), endosialin (CD248), epidermal growth factor receptor variant III (EGFRvIII), tumor-associated antigen L6 (TAL6), SAS, CD63, TAG72, Thomsen-Friedenreich antigen (TF-antigen), insulin-like growth factor I receptor (IGF-IR), Cora antigen, CD7, CD22, CD70 (e.g., its natural ligand, CD27 rather than an antibody fragment), CD79a, CD79b, G250, MT-MMPs, alpha-fetoprotein (AFP), VEGFR1, VEGFR2, DLK1, SP17, ROR1, EphA2, ENPP3, glypican 3 (GPC3), and TPBG/5T4 (trophoblast glycoprotein). The tumor-specific marker or the antigen of the target cell can be selected from alpha 4 integrin, Ang2, CEACAM5, cMET, CTLA4, FOLR1, EpCAM (epithelial cell adhesion molecule), CD19, HER2, HER2 neu, HER3, HER4, HER1 (EGFR), PD-L1, PSMA, CEA, TROP-2, MUC1(mucin), Lewis-Y, CD20, CD33, CD38, mesothelin, CD70 (e.g., its natural ligand, CD27 rather than an antibody fragment), VEGFR1, VEGFR2, ROR1, EphA2, ENPP3, glypican 3 (GPC3), and TPBG/5T4 (trophoblast glycoprotein). The tumor-specific marker or the antigen of the target cell can be any one set forth in the “Target” column of Table 6. The binding domain with binding affinity to the tumor-specific marker or the target cell antigen can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99%, or 100%, sequence identity to any one of the paired VL and VH sequences set forth in the “VH Sequences” and “VL Sequences” columns of Table 6.

TABLE 6
Anti-target Cell Monoclonal Antibodies and Sequences

| Trade | Antibody | | SEQ | | SEQ | |
|---|---|---|---|---|---|---|
| Name | Name | Target | ID NO: | VH Sequence | ID NO: | VL Sequence |
| Tysabri™ | natalizumab | Alpha | 555 | QVQLVQSGAEVKKPG | 654 | DIQMTQSPSSLSASVG |
| | | 4 | | ASVKVSCKASGFNIKD | | DRVTITCKTSQDINK |
| | | Integrin | | TYIHWVRQAPGQRLE | | YMAWYQQTPGKAPR |
| | | | | WMGRIDPANGYTKY | | LLIHYTSALQPGIPSR |
| | | | | DPKFQGRVTITADTSA | | FSGSGSGRDYTFTISS |
| | | | | STAYMELSSLRSEDTA | | LQPEDIATYYCLQYD |
| | | | | VYYCAREGYYGNYG | | NLWTFGQGTKVEIK |
| | | | | VYAMDYWGQGTLVT | | |
| | | | | VSS | | |
| REGN910 | nesvacumab | Ang2 | 556 | EVQLVESGGGLVQPGG | 655 | EIVLTQSPGTLSLSPG |
| | | | | SLRLSCAASGFTFSSY | | ERATLSCRASQSVSS |
| | | | | DIHWVRQATGKGLEW | | TYLAWYQQKPGQAP |
| | | | | VSAIGPAGDTYYPGSV | | RLLIYGASSRATGIPD |
| | | | | KGRFTISRENAKNSLY | | RFSGSGSGTDFTLTIS |
| | | | | LQMNSLRAGDTAVYY | | RLEPEDFAVYYCQH |
| | | | | CARGLITFGGLIAPFD | | YDNSQTFGQGTKVEI |
| | | | | YWGQGTLVTVSS | | K |
| hMFE23 | | CEA | 557 | QVKLEQSGAEVVKPG | 656 | ENVLTQSPSSMSASV |
| | | | | ASVKLSCKASGFNIKD | | GDRVNIACSASSSVS |
| | | | | SYMHWLRQGPGQRLE | | YMHWFQQKPGKSPK |
| | | | | WIGWIDPENGDTEYAP | | LWIYSTSNLASGVPS |
| | | | | KFQGKATFTTDTSANT | | RFSGSGSGTDYSLTIS |
| | | | | AYLGLSSLRPEDTAVY | | SMQPEDAATYYCQQ |
| | | | | YCNEGTPTGPYYFDY | | RSSYPLTFGGGTKLEI |
| | | | | WGQGTLVTVSS | | K |
| M5A | | CEA | 558 | EVQLVESGGGLVQPGG | 657 | DIQLTQSPSSLSASVG |
| (humanized | | | | SLRLSCAASGFNIKDT | | DRVTITCRAGESVDI |
| T84.66) | | | | YMHWVRQAPGKGLE | | FGVGFLHWYQQKPG |
| | | | | WVARIDPANGNSKYA | | KAPKLLIYRASNLES |
| | | | | DSVKGRFTISADTSKN | | GVPSRFSGSGSRTDFT |
| | | | | TAYLQMNSLRAEDTA | | LTISSLQPEDFATYYC |
| | | | | VYYCAPFGYYVSDYA | | QQTNEDPYTFGQGT |
| | | | | MAYWGQGTLVTVSS | | KVEIK |
| M5B | | CEA | 559 | EVQLVESGGGLVQPGG | 658 | DIQLTQSPSSLSASVG |
| (humanized | | | | SLRLSCAASGFNIKDT | | DRVTITCRAGESVDI |
| T84.66) | | | | YMHWVRQAPGKGLE | | FGVGFLHWYQQKPG |
| | | | | WVARIDPANGNSKYV | | KAPKLLIYRASNLES |
| | | | | PKFQGRATISADTSKN | | GVPSRFSGSGSRTDFT |
| | | | | TAYLQMNSLRAEDTA | | LTISSLQPEDFATYYC |
| | | | | VYYCAPFGYYVSDYA | | QQTNEDPYTFGQGT |
| | | | | MAYWGQGTLVTVSS | | KVEIK |
| CEA-Cide | Labetuzumab | CEAC | 560 | EVQLVESGGGVVQPG | 659 | DIQLTQSPSSLSASVG |
| | (MN-14) | AM5 | | RSLRLSCSASGFDFTTY | | DRVTITCKASQDVGT |
| | | | | WMSWVRQAPGKGLE | | SVAWYQQKPGKAPK |
| | | | | WIGEIHPDSSTINYAPS | | LLIYWTSTRHTGVPS |
| | | | | LKDRFTISRDNAKNTL | | RFSGSGSGTDFTFTIS |
| | | | | FLQMDSLRPEDTGVYF | | SLQPEDIATYYCQQY |
| | | | | CASLYFGFPWFAYWG | | SLYRSFGQGTKVEIK |
| | | | | QGTPVTVSS | | |
| CEA-Scan | arcitumomab | CEAC | 561 | EVKLVESGGGLVQPGG | 660 | QTVLSQSPAILSASPG |
| | | AM5 | | SLRLSCATSGFTFTDY | | EKVTMTCRASSSVTY |
| | | | | YMNWVRQPPGKALE | | IHWYQQKPGSSPKS |
| | | | | WLGFIGNKANGYTTE | | WIYATSNLASGVPAR |
| | | | | YSASVKGRFTISRDKS | | FSGSGSGTSYSLTISR |
| | | | | QSILYLQMNTLRAEDS | | VEAEDAATYYCQHW |
| | | | | ATYYCTRDRGLRFYF | | SSKPPTFGGGTKLEIK |
| | | | | DYWGQGTTLTVSS | | R |
| MT110 | | CEAC | 562 | EVQLVESGGGLVQPGR | 661 | QAVLTQPASLSASPG |
| | | AM5 | | SLRLSCAASGFTVSSY | | ASASLTCTLRRGINV |
| | | | | WMHWVRQAPGKGLE | | GAYSIYWYQQKPGSP |
| | | | | WVGFIRNKANGGTTE | | PQYLLRYKSDSDKQ |
| | | | | YAASVKGRFTISRDDS | | QGSGVSSRFSASKDA |
| | | | | KNTLYLQMNSLRAED | | SANAGILLISGLQSED |
| | | | | TAVYYCARDRGLRFY | | EADYYCMIWHSGAS |
| | | | | FDYWGQGTTVTVSS | | AVFGGGTKLTVL |
| MT103 | blinatumom | CD19 | 563 | QVQLQQSGAELVRPGS | 662 | DIQLTQSPASLAVSLG |
| | | | | SVKISCKASGYAFSSY | | QRATISCKASQSVDY |
| | | | | WMNWVKQRPGQGLE | | DGDSYLNWYQQIPG |
| | at | | | WIGQIWPGDGDTNYN | | QPPKLLIYDASNLVS |
| | | | | GKFKGKATLTADESSS | | GIPPRFSGSGSGTDFT |
| | | | | TAYMQLSSLASEDSAV | | LNIHPVEKVDAATYH |
| | | | | YFCARRETTTVGRYY | | CQQSTEDPWTFGGG |
| | | | | YAMDYWGQGTTVTVS | | TKLEIK |
| | | | | S | | |
| Arzerra | ofatumumab | CD20 | 564 | EVQLVESGGGLVQPGR | 663 | EIVLTQSPATLSLSPG |
| | | | | SLRLSCAASGFTFNDY | | ERATLSCRASQSVSS |
| | | | | AMHWVRQAPGKGLE | | YLAWYQQKPGQAPR |
| | | | | WVSTISWNSGSIGYAD | | LLIYDASNRATGIPAR |
| | | | | SVKGRFTISRDNAKKS | | FSGSGSGTDFTLTISS |
| | | | | LYLQMNSLRAEDTAL | | LEPEDFAVYYCQQRS |
| | | | | YYCAKDIQYGNYYYG | | NWPITFGQGTRLEIK |
| | | | | MDVWGQGTTVTVSS | | |
| Bexxar™ | tositumomab | CD20 | 565 | QAYLQQSGAELVRPG | 664 | QIVLSQSPAILSASPG |
| | | | | ASVKMSCKASGYTFTS | | EKVTMTCRASSSVSY |
| | | | | YNMHWVKQTPRQGLE | | MHWYQQKPGSSPKP |
| | | | | WIGAIYPGNGDTSYN | | WIYAPSNLASGVPAR |
| | | | | QKFKGKATLTVDKSS | | FSGSGSGTSYSLTISR |
| | | | | STAYMQLSSLTSEDSA | | VEAEDAATYYCQQW |
| | | | | VYFCARVVYYSNSYW | | SFNPPTFGAGTKLEL |
| | | | | YFDVWGTGTTVTVSG | | K |
| GAZYVA | Obinutuzumab | CD20 | 566 | QVQLVQSGAEVKKPG | 665 | DIVMTQTPLSLPVTPG |
| | | | | SSVKVSCKASGYAFSY | | EPASISCRSSKSLLHS |
| | | | | SWINWVRQAPGQGLE | | NGITYLYWYLQKPG |
| | | | | WMGRIFPGDGDTDYN | | QSPQLLIYQMSNLVS |
| | | | | GKFKGRVTITADKSTS | | GVPDRFSGSGSGTDF |
| | | | | TAYMELSSLRSEDTAV | | TLKISRVEAEDVGVY |
| | | | | YYCARNVFDGYWLV | | YCAQNLELPYTFGG |
| | | | | YWGQGTLVTVSS | | GTKVEIK |
| | Ocrelizuma | CD20 | 567 | EVQLVESGGGLVQPGG | 666 | DIQMTQSPSSLSASVG |
| | b/2H7 v16 | | | SLRLSCAASGYTFTSY | | DRVTITCRASSSVSY |
| | | | | NMHWVRQAPGKGLE | | MHWYQQKPGKAPKP |
| | | | | WVGAIYPGNGDTSYN | | LIYAPSNLASGVPSRF |
| | | | | QKFKGRFTISVDKSKN | | SGSGSGTDFTLTISSL |
| | | | | TLYLQMNSLRAEDTA | | QPEDFATYYCQQWS |
| | | | | VYYCARVVYYSNSYW | | FNPPTFGQGTKVEIK |
| | | | | YFDVWGQGTLVTVSS | | |
| Rituxan™ | rituximab | CD20 | 568 | QVQLQQPGAELVKPG | 667 | QIVLSQSPAILSASPG |
| | | | | ASVKMSCKASGYTFT | | EKVTMTCRASSSVSY |
| | | | | SYNMHWVKQTPGRGL | | IHWFQQKPGSSPKPW |
| | | | | EWIGAIYPGNGDTSYN | | IYATSNLASGVPVRFS |
| | | | | QKFKGKATLTADKSSS | | GSGSGTSYSLTISRVE |
| | | | | TAYMQLSSLTSEDSAV | | AEDAATYYCQQWTS |
| | | | | YYCARSTYYGGDWY | | NPPTFGGGTKLEIK |
| | | | | FNVWGAGTTVTVSA | | |
| Zevalin™ | ibritumomab | CD20 | 569 | QAYLQQSGAELVRPG | 668 | QIVLSQSPAILSASPG |
| | tieuxetan | | | ASVKMSCKASGYTFT | | EKVTMTCRASSSVSY |
| | | | | SYNMHWVKQTPRQGL | | MHWYQQKPGSSPKP |
| | | | | EWIGAIYPGNGDTSYN | | WIYAPSNLASGVPAR |
| | | | | QKFKGKATLTVDKSSS | | FSGSGSGTSYSLTISR |
| | | | | TAYMQLSSLTSEDSAV | | VEAEDAATYYCQQW |
| | | | | YFCARVVYYSNSYWY | | SFNPPTFGAGTKLEL |
| | | | | FDVWGTGTTVTVSA | | K |
| Mylotarg | Gemtuzumab | CD33 | 570 | QLVQSGAEVKKPGSSV | 669 | DIQLTQSPSTLSASVG |
| | (hP67.6) | | | KVSCKASGYTITDSNI | | DRVTITCRASESLDN |
| | | | | HWVRQAPGQSLEWIG | | YGIRFLTWFQQKPG |
| | | | | YIYPYNGGTDYNQKF | | KAPKLLMYAASNQG |
| | | | | KNRATLTVDNPTNTA | | SGVPSRFSGSGSGTEF |
| | | | | YMELSSLRSEDTDFYY | | TLTISSLQPDDFATYY |
| | | | | CVNGNPWLAYWGQG | | CQQTKEVPWSFGQG |
| | | | | TLVTVSS | | TKVEVK |
| Daratumumab | | CD38 | 571 | EVQLLESGGGLVQPGG | 670 | EIVLTQSPATLSLSPG |
| | | | | SLRLSCAVSGFTFNSF | | ERATLSCRASQSVSS |
| | | | | AMSWVRQAPGKGLE | | YLAWYQQKPGQAPR |
| | | | | WVSAISGSGGGTYYA | | LLIYDASNRATGIPAR |
| | | | | DSVKGRFTISRDNSKN | | FSGSGSGTDFTLTISS |
| | | | | TLYLQMNSLRAEDTA | | LEPEDFAVYYCQQRS |
| | | | | VYFCAKDKILWFGEP | | NWPPTFGQGTKVEIK |
| | | | | VFDYWGQGTLVTVSS | | |
| | 1F6 | CD70 | 572 | QIQLVQSGPEVKKPGE | 671 | DIVLTQSPASLAVSLG |
| | | | | TVKISCKASGYTFTNY | | QRATISCRASKSVSTS |
| | | | | GMNWVKQAPGKGLK | | GYSFMHWYQQKPG |
| | | | | WMGWINTYTGEPTY | | QPPKLLIYLASNLESG |
| | | | | ADAFKGRFAFSLETSA | | VPARFSGSGSGTDFT |
| | | | | STAYLQINNLKNEDTA | | LNIHPVEEEDAATY |
| | | | | TYFCARDYGDYGMDY | | YCQHSREVPWTFGG |
| | | | | WGQGTSVTVSS | | GTKLEIK |
| | 2F2 | CD70 | 573 | QVQLQQSGTELMTPG | 672 | DIVLTQSPASLTVSLG |
| | | | | ASVTMSCKTSGYTFST | | QKTTISCRASKSVSTS |
| | | | | YWIEWVKQRPGHGLE | | GYSFMHWYQLKPGQ |
| | | | | WIGEILGPSGYTDYNE | | SPKLLIYLASDLPSGV |
| | | | | KFKAKATFTADTSSNT | | PARFSGSGSGTDFTL |
| | | | | AYMQLSSLASEDSAVY | | KIHPVEEEDAATY |
| | | | | YCARWDRLYAMDYW | | YCQHSREIPYTFGGG |
| | | | | GGGTSVTVSS | | TKLEIT |
| | 2H5 | CD70 | 574 | QVQLVESGGGVVQPG | 673 | EIVLTQSPATLSLSPG |
| | | | | RSLRLSCAASGFTFSSY | | ERATLSCRASQSVSS |
| | | | | IMHWVRQAPGKGLEW | | YLAWYQQKPGQAPR |
| | | | | VAVISYDGRNKYYAD | | LLIYDASNRATGIPAR |
| | | | | SVKGRFTISRDNSKNT | | FSGSGSGTDFTLTISS |
| | | | | LYLQMNSLRAED | | LEPEDFAVYYCQQ |
| | | | | TAVYYCARDTDGYDF | | RTNWPLTFGGGTKV |
| | | | | DYWGQGTLVTVSS | | EIK |
| | 10B4 | CD70 | 575 | QIQLVESGGGVVQPGR | 674 | AIQLTQSPSSLSASVG |
| | | | | SLRLSCAASGFTFGYY | | DRVTITCRASQGISSA |
| | | | | AMHWVRQAPGKGLE | | LAWYQQKPGKAPKF |
| | | | | WVAVISYDGSIKYYA | | LIYDASSLESGVPSRF |
| | | | | DSVKGRFTISRDNSKN | | SGSGSGTDFTLTISSL |
| | | | | TLYLQMNSLRAED | | QPEDFATYYCQQ |
| | | | | TAVYYCAREGPYSNY | | FNSYPFTFGPGTKVD |
| | | | | LDYWGQGTLVTVSS | | IK |
| | 8B5 | CD70 | 576 | QVQLVESGGGVVQPG | 675 | DIQMTQSPSSLSASVG |
| | | | | RSLRLSCATSGFTFSDY | | DRVTITCRASQGISS |
| | | | | GMHWVRQAPGKGLE | | WLAWYQQKPEKAPK |
| | | | | WVAVIWYDGSNKYY | | SLIYAASSLQSGVPSR |
| | | | | ADSVKGRFTISRDNSK | | FSGSGSGTDFTLTISS |
| | | | | KTLSLQMNSLRAED | | LQPEDFATYYCQQ |
| | | | | TAVYYCARDSIMVRG | | YNSYPLTFGGGTKVE |
| | | | | DYWGQGTLVTVSS | | IK |
| | 18E7 | CD70 | 577 | QVQLVESGGGVVQPG | 676 | DIQMTQSPSSLSASVG |
| | | | | RSLRLSCAASGFTFSD | | DRVTITCRASQGISS |
| | | | | HGMHWVRQAPGKGL | | WLAWYQQKPEKAPK |
| | | | | EWVAVIWYDGSNKY | | SLIYAASSLQSGVPSR |
| | | | | YADSVKGRFTISRDNS | | FSGSGSGTDFTLTISS |
| | | | | KNTLYLQMNSLRAED | | LQPEDFATYYCQQ |
| | | | | TAVYYCARDSIMVRG | | YNSYPLTFGGGTKVE |
| | | | | DYWGQGTLVTVSS | | IK |
| | 69A7 | CD70 | 578 | QVQLQESGPGLVKPSE | 677 | EIVLTQSPATLSLSPG |
| | | | | TLSLTCTVSGGSVSSD | | ERATLSCRASQSVSS |
| | | | | YYYWSWIRQPPGKGL | | YLAWYQQKPGQAPR |
| | | | | EWLGYIYYSGSTNYNP | | LLIFDASNRATGIPAR |
| | | | | SLKSRVTISVDTSKNQF | | FSGSGSGTDFTLTISS |
| | | | | SLKLRSVTTA | | LEPEDFAVYYCQQ |
| | | | | DTAVYYCARGDGDYG | | RSNWPLTFGGGTKV |
| | | | | GNCFDYWGQGTLVTV | | EIK |
| | | | | SS | | |
| CE- | | cMET | 579 | QVQLVQSGAEVKKPG | 678 | DIQMTQSPSSVSASV |
| 355621 | | | | ASVKVSCKASGYTFTS | | GDRVTITCRASQGIN |
| | | | | YGFSWVRQAPGQGLE | | TWLAWYQQKPGKA |
| | | | | WMGWISASNGNTYY | | PKLLIYAASSLKSGVP |
| | | | | AQKLQGRVTMTTDTS | | SRFSGSGSGTDFTLTI |
| | | | | TSTAYMELRSLRSDDT | | SSLQPEDFATYYCQQ |
| | | | | AVYYCARVYADYADY | | ANSFPLTFGGGTKVE |
| | | | | WGQGTLVTVSS | | IK |
| LY28753 | emibetuzumab | cMET | 580 | QVQLVQSGAEVKKPG | 679 | DIQMTQSPSSLSASVG |
| 58 | | | | ASVKVSCKASGYTFT | | DRVTITCSVSSSVSSI |
| | | | | DYYMHWVRQAPGQG | | YLHWYQQKPGKAPK |
| | | | | LEWMGRVNPNRRGTT | | LLIYSTSNLASGVPSR |
| | | | | YNQKFEGRVTMTTDTS | | FSGSGSGTDFTLTISS |
| | | | | TSTAYMELRSLRSDDT | | LQPEDFATYYCQVYS |
| | | | | AVYYCARANWLDYW | | GYPLTFGGGTKVEIK |
| | | | | GQGTTVTVSS | | |
| MetMAb | onartuzumab | cMET | 581 | EVQLVESGGGLVQPGG | 680 | DIQMTQSPSSLSASVG |
| | | | | SLRLSCAASGYTFTSY | | DRVTITCKSSQSLLY |
| | | | | WLHWVRQAPGKGLE | | TSSQKNYLAWYQQK |
| | | | | WVGMIDPSNSDTRFN | | PGKAPKLLIYWASTR |
| | | | | PNFKDRFTISADTSKN | | ESGVPSRFSGSGSGT |
| | | | | TAYLQMNSLRAEDTA | | DFTLTISSLQPEDFAT |
| | | | | VYYCATYRSYVTPLD | | YYCQQYYAYPWTFG |
| | | | | YWGQGTLVTVSS | | QGTKVEIK |
| | tremelimumab | CTLA | 582 | QVQLVESGGGVVQPG | 681 | DIQMTQSPSSLSASVG |
| | (CP-675206, | 4 | | RSLRLSCAASGFTFSS | | DRVTITCRASQSINSY |
| | or 11.2.1) | | | YGMHWVRQAPGKGL | | LDWYQQKPGKAPKL |
| | | | | EWVAVIWYDGSNKY | | LIYAASSLQSGVPSRF |
| | | | | YADSVKGRFTISRDNS | | SGSGSGTDFTLTISSL |
| | | | | KNTLYLQMNSLRAED | | QPEDFATYYCQQYY |
| | | | | TAVYYCARDPRGATL | | STPFTFGPGTKVEIK |
| | | | | YYYYYGMDVWGQGT | | |
| | | | | TVTVSS | | |
| Yervoy | Ipilimumab | CTLA | 583 | QVQLVESGGGVVQPG | 682 | EIVLTQSPGTLSLSPG |
| | 10D1 | 4 | | RSLRLSCAASGFTFSSY | | ERATLSCRASQSVGS |
| | | | | TMHWVRQAPGKGLE | | SYLAWYQQKPGQAP |
| | | | | WVTFISYDGNNKYYA | | RLLIYGAFSRATGIPD |
| | | | | DSVKGRFTISRDNSKN | | RFSGSGSGTDFTLTIS |
| | | | | TLYLQMNSLRAEDTAI | | RLEPEDFAVYYCQQ |
| | | | | YYCARTGWLGPFDY | | YGSSPWTFGQGTKV |
| | | | | WGQGTLVTVSS | | EIK |
| AGS16F | H16-7.8 | ENPP3 | 584 | QVQLQESGPGLVKPSQ | 683 | EIVLTQSPDFQSVTPK |
| | | | | TLSLTCTVSGGSISSGG | | EKVTITCRASQSIGIS |
| | | | | YYWSWIRQHPGKGLE | | LHWYQQKPDQSPKL |
| | | | | WIGIIYYSGSTYYNPSL | | LIKYASQSFSGVPSRF |
| | | | | KSRVTISVDTSKNQFSL | | SGSGSGTDFTLTINSL |
| | | | | KLNSVTAADTAVFYC | | EAEDAATYYCHQSR |
| | | | | ARVAIVTTIPGGMDV | | SFPWTFGQGTKVEIK |
| | | | | WGQGTTVTVSS | | |
| MT110 | solitomab | EpCA | 585 | EVQLLEQSGAELVRPG | 684 | ELVMTQSPSSLTVTA |
| | | M | | TSVKISCKASGYAFTN | | GEKVTMSCKSSQSLL |
| | | | | YWLGWVKQRPGHGL | | NSGNQKNYLTWYQ |
| | | | | EWIGDIFPGSGNIHYN | | QKPGQPPKLLIYWAS |
| | | | | EKFKGKATLTADKSSS | | TRESGVPDRFTGSGS |
| | | | | TAYMQLSSLTFEDSAV | | GTDFTLTISSVQAEDL |
| | | | | YFCARLRNWDEPMD | | AVYYCQNDYSYPLT |
| | | | | YWGQGTTVTVSS | | FGAGTKLEIK |
| MT201 | Adecatumumab | EpCA | 586 | EVQLLESGGGVVQPGR | 685 | ELQMTQSPSSLSASV |
| | | M | | SLRLSCAASGFTFSSYG | | GDRVTITCRTSQSISS |
| | | | | MHWVRQAPGKGLEW | | YLNWYQQKPGQPPK |
| | | | | VAVISYDGSNKYYAD | | LLIYWASTRESGVPD |
| | | | | SVKGRFTISRDNSKNT | | RFSGSGSGTDFTLTIS |
| | | | | LYLQMNSLRAEDTAV | | SLQPEDSATYYCQQS |
| | | | | YYCAKDMGWGSGW | | YDIPYTFGQGTKLEI |
| | | | | RPYYYYGMDVWGQG | | K |
| | | | | TTVTVSS | | |
| Panorex | Edrecolomab | EpCA | 587 | QVQLQQSGAELVRPGT | 686 | NIVMTQSPKSMSMSV |
| | Mab CO17- | M | | SVKVSCKASGYAFTN | | GERVTLTCKASENVV |
| | 1A | | | YLIEWVKQRPGQGLE | | TYVSWYQQKPEQSP |
| | | | | WIGVINPGSGGTNYNE | | KLLIYGASNRYTGVP |
| | | | | KFKGKATLTADKSSST | | DRFTGSGSATDFTLTI |
| | | | | AYMQLSSLTSDDSAVY | | SSVQAEDLADYHCG |
| | | | | FCARDGPWFAYWGQ | | QGYSYPYTFGGGTK |
| | | | | GTLVTVSA | | LEIK |
| | tucotuzumab | EpCA | 588 | QIQLVQSGPELKKPGE | 687 | QILLTQSPAIMSASPG |
| | | M | | TVKISCKASGYTFTNY | | EKVTMTCSASSSVSY |
| | | | | GMNWVRQAPGKGLK | | MLWYQQKPGSSPKP |
| | | | | WMGWINTYTGEPTY | | WIFDTSNLASGFPAR |
| | | | | ADDFKGRFVFSLETSA | | FSGSGSGTSYSLIISSM |
| | | | | STAFLQLNNLRSEDTA | | EAEDAATYYCHQRS |
| | | | | TYFCVRFISKGDYWGQ | | GYPYTFGGGTKLEIK |
| | | | | GTSVTVSS | | |
| UBS-54 | | EpCA | 589 | VQLQQSDAELVKPGAS | 688 | DIVMTQSPDSLAVSL |
| | | M | | VKISCKASGYTFTDHA | | GERATINCKSSQSVL |
| | | | | IHWVKQNPEQGLEWI | | YSSNNKNYLAWYQQ |
| | | | | GYFSPGNDDFKYNER | | KPGQPPKLLIYWAST |
| | | | | FKGKATLTADKSSSTA | | RESGVPDRFSGSGSG |
| | | | | YVQLNSLTSEDSAVYF | | TDFTLTISSLQAEDVA |
| | | | | CTRSLNMAYWGQGTS | | VYYCQQYYSYPLTF |
| | | | | VTVSS | | GGGTKVKES |
| 3622W94 | 323/A3 | EpCA | 590 | EVQLVQSGPEVKKPGA | 689 | DIVMTQSPLSLPVTPG |
| | | M | | SVKVSCKASGYTFTN | | EPASISCRSSINKKGS |
| | | | | YGMNWVRQAPGQGL | | NGITYLYWYLQKPG |
| | | | | EWMGWINTYTGEPTY | | QSPQLLIYQMSNLAS |
| | | | | GEDFKGRFAFSLDTSA | | GVPDRFSGSGSGTDF |
| | | | | STAYMELSSLRSEDTA | | TLKISRVEAEDVGVY |
| | | | | VYFCARFGNYVDYWG | | YCAQNLEIPRTFGQG |
| | | | | QGSLVTVSS | | TKVEIK |
| 4D5MOC | | EpCA | 591 | EVQLVQSGPGLVQPGG | 690 | DIQMTQSPSSLSASVG |
| Bv2 | | M | | SVRISCAASGYTFTNY | | DRVTITCRSTKSLLH |
| | | | | GMNWVKQAPGKGLE | | SNGITYLYWYQQKP |
| | | | | WMGWINTYTGESTY | | GKAPKLLIYQMSNLA |
| | | | | ADSFKGRFTFSLDTSA | | SGVPSRFSSSGSGTDF |
| | | | | SAAYLQINSLRAEDTA | | TLTISSLQPEDFATYY |
| | | | | VYYCARFAIKGDYWG | | CAQNLEIPRTFGQGT |
| | | | | QGTLLTVSS | | KVEIK |
| 4D5MOC | | EpCA | 592 | EVQLVQSGPGLVQPGG | 691 | DIQMTQSPSSLSASVG |
| B | | M | | SVRISCAASGYTFTNY | | DRVTITCRSTKSLLH |
| | | | | GMNWVKQAPGKGLE | | SNGITYLYWYQQKP |
| | | | | WMGWINTYTGESTY | | GKAPKLLIYQMSNLA |
| | | | | ADSFKGRFTFSLDTSA | | SGVPSRFSSSGSGTDF |
| | | | | SAAYLQINSLRAEDTA | | TLTISSLQPEDFATYY |
| | | | | VYYCARFAIKGDYWG | | CAQNLEIPRTFGQGT |
| | | | | QGTLLTVSS | | KVELK |
| MEDI- | 1C1 | EphA2 | 593 | EVQLLESGGGLVQPGG | 692 | DIQMTQSPSSLSASVG |
| 547 | | | | SLRLSCAASGFTFSHY | | DRVTITCRASQSIST |
| | | | | MMAWVRQAPGKGLE | | WLAWYQQKPGKAP |
| | | | | WVSRIGPSGGPTHYA | | KLLIYKASNLHTGVP |
| | | | | DSVKGRFTISRDNSKN | | SRFSGSGSGTEFSLTIS |
| | | | | TLYLQMNSLRAEDTA | | GLQPDDFATYYCQQ |
| | | | | VYYCAGYDSGYDYVA | | YNSYSRTFGQGTKVE |
| | | | | VAGPAEYFQHWGQG | | IK |
| | | | | TLVTVSS | | |
| MORAb- | farletuzumab | FOLR1 | 594 | EVQLVESGGGVVQPG | 693 | DIQLTQSPSSLSASVG |
| 003 | | | | RSLRLSCSASGFTFSG | | DRVTITCSVSSSISSN |
| | | | | YGLSWVRQAPGKGLE | | NLHWYQQKPGKAPK |
| | | | | WVAMISSGGSYTYYA | | PWIYGTSNLASGVPS |
| | | | | DSVKGRFAISRDNAKN | | RFSGSGSGTDYTFTIS |
| | | | | TLFLQMDSLRPEDTGV | | SLQPEDIATYYCQQW |
| | | | | YFCARHGDDPAWFAY | | SSYPYMYTFGQGTK |
| | | | | WGQGTPVTVSS | | VEIK |
| M9346A | huMOV19 | FOLR1 | 595 | QVQLVQSGAEVVKPG | 694 | DIVLTQSPLSLAVSLG |
| | (vLCv1.00) | | | ASVKISCKASGYTFTG | | QPAIISCKASQSVSFA |
| | | | | YFMNWVKQSPGQSLE | | GTSLMHWYHQKPG |
| | | | | WIGRIHPYDGDTFYN | | QQPRLLIYRASNLEA |
| | | | | QKFQGKATLTVDKSS | | GVPDRFSGSGSKTDF |
| | | | | NTAHMELLSLTSEDFA | | TLNISPVEAEDAATY |
| | | | | VYYCTRYDGSRAMDY | | YCQQSREYPYTFGG |
| | | | | WGQGTTVTVSS | | GTKLEIK |
| M9346A | huMOV19 | FOLR1 | 596 | QVQLVQSGAEVVKPG | 695 | DIVLTQSPLSLAVSLG |
| | (vLCv1.60) | | | ASVKISCKASGYTFTG | | QPAIISCKASQSVSFA |
| | | | | YFMNWVKQSPGQSLE | | GTSLMHWYHQKPG |
| | | | | WIGRIHPYDGDTFYN | | QQPRLLIYRASNLEA |
| | | | | QKFQGKATLTVDKSS | | GVPDRFSGSGSKTDF |
| | | | | NTAHMELLSLTSEDFA | | TLTISPVEAEDAATY |
| | | | | VYYCTRYDGSRAMDY | | YCQQSREYPYTFGG |
| | | | | WGQGTTVTVSS | | GTKLEIK |
| 26B3.F2 | | FOLR1 | 597 | GPELVKPGASVKISCK | 696 | PASLSASVGETVTITC |
| | | | | ASDYSFTGYFMNWVM | | RTSENIFSYLAWYQQ |
| | | | | QSHGKSLEWIGRIFPY | | KQGISPQLLVYNAKT |
| | | | | NGDTFYNQKFKGRAT | | LAEGVPSRFSGSGSG |
| | | | | LTVDKSSSTAHMELRS | | TQFSLKINSLQPEDFG |
| | | | | LASEDSAVYFCARGTH | | SYYCQHHYAFPWTF |
| | | | | YFDYWGQGTTLTVSS | | GGGSKLEIK |
| RG7686 | GC33 | GPC3 | 598 | QVQLVQSGAEVKKPG | 697 | DVVMTQSPLSLPVTP |
| | | | | ASVKVSCKASGYTFTD | | GEPASISCRSSQSLVH |
| | | | | YEMHWVRQAPGQGL | | SNGNTYLHWYLQKP |
| | | | | EWMGALDPKTGDTA | | GQSPQLLIYKVSNRF |
| | | | | YSQKFKGRVTLTADK | | SGVPDRFSGSGSGTD |
| | | | | STSTAYMELSSLTSED | | FTLKISRVEAEDVGV |
| | | | | TAVYYCTRFYSYTYW | | YYCSQNTHVPPTFG |
| | | | | GQGTLVTVSS | | QGTKLEIK |
| | 4A6 | GPC3 | 599 | EVQLVQSGAEVKKPGE | 698 | EIVLTQSPGTLSLSPG |
| | | | | SLKISCKGSGYSFTSY | | ERATLSCRAVQSVSS |
| | | | | WIAWVRQMPGKGLE | | SYLAWYQQKPGQAP |
| | | | | WMGIIFPGDSDTRYSP | | RLLIYGASSRATGIPD |
| | | | | SFQGQVTISADRSIRTA | | RFSGSGSGTDFTLTIS |
| | | | | YLQWSSLKASD | | RLEPEDFAVYYCQ |
| | | | | TALYYCARTREGYFD | | QYGSSPTFGGGTKVE |
| | | | | YWGQGTLVTVSS | | IK |
| | 11E7 | GPC3 | 600 | EVQLVQSGAEVKKPGE | 699 | EIVLTQSPGTLSLSPG |
| | | | | SLKISCKGSGYSFTNY | | ERATLSCRASQSVSS |
| | | | | WIAWVRQMPGKGLE | | SYLAWYQQKPGQAP |
| | | | | WMGIIYPGDSDTRYSP | | RLLIYGASSRATGIPD |
| | | | | SFQGQVTISADKSIRTA | | RFSGSGSGTDFTLTIS |
| | | | | YLQWSSLKASD | | RLEPEDFAVYYCQ |
| | | | | TAMYYCARTREGYFD | | QYGSSPTFGGGTKVE |
| | | | | YWGQGTLVTVSS | | IK |
| | 16D10 | GPC3 | 601 | EVQLVQSGADVTKPGE | 700 | EILLTQSPGTLSLSPG |
| | | | | SLKISCKVSGYRFTNY | | ERATLSCRASQSVSS |
| | | | | WIGWMRQMSGKGLE | | SYLAWYQQKPGQAP |
| | | | | WMGIIYPGDSDTRYSP | | RLLIYGASSRATGIPD |
| | | | | SFQGHVTISADKSINTA | | RFSGSGSGTDFTLTIS |
| | | | | YLRWSSLKASD | | RLEPEDFAVYYCQ |
| | | | | TAIYYCARTREGFFDY | | QYGSSPTFGQGTKVE |
| | | | | WGQGTPVTVSS | | IK |
| AMG-595 | | HER1 | 602 | QVQLVESGGGVVQSG | 701 | DTVMTQTPLSSHVTL |
| | | (EGFR) | | RSLRLSCAASGFTFRN | | GQPASISCRSSQSLV |
| | | | | YGMHWVRQAPGKGL | | HSDGNTYLSWLQQR |
| | | | | EWVAVIWYDGSDKY | | PGQPPRLLIYRISRRF |
| | | | | YADSVRGRFTISRDNS | | SGVPDRFSGSGAGTD |
| | | | | KNTLYLQMNSLRAED | | FTLEISRVEAEDVGV |
| | | | | TAVYYCARDGYDILT | | YYCMQSTHVPRTFG |
| | | | | GNPRDFDYWGQGTLV | | QGTKVEIK |
| | | | | TVSS | | |
| Erubitux™ | cetutximab | HER1 | 603 | QVQLKQSGPGLVQPSQ | 702 | DILLTQSPVILSVSPGE |
| | | (EGFR) | | SLSITCTVSGFSLTNYG | | RVSFSCRASQSIGTNI |
| | | | | VHWVRQSPGKGLEWL | | HWYQQRTNGSPRLLI |
| | | | | GVIWSGGNTDYNTPF | | KYASESISGIPSRFSGS |
| | | | | TSRLSINKDNSKSQVFF | | GSGTDFTLSINSVESE |
| | | | | KMNSLQSNDTAIYYCA | | DIADYYCQQNNNWP |
| | | | | RALTYYDYEFAYWGQ | | TTFGAGTKLELK |
| | | | | GTLVTVSA | | |
| GA201 | Imgatuzumab | HER1 | 604 | QVQLVQSGAEVKKPG | 703 | DIQMTQSPSSLSASVG |
| | | (EGFR) | | SSVKVSCKASGFTFTD | | DRVTITCRASQGINN |
| | | | | YKIHWVRQAPGQGLE | | YLNWYQQKPGKAPK |
| | | | | WMGYFNPNSGYSTYA | | RLIYNTNNLQTGVPS |
| | | | | QKFQGRVTITADKSTS | | RFSGSGSGTEFTLTISS |
| | | | | TAYMELSSLRSEDTAV | | LQPEDFATYYCLQH |
| | | | | YYCARLSPGGYYVMD | | NSFPTFGQGTKLEIK |
| | | | | AWGQGTTVTVSS | | |
| Humax | zalutumumab | HER1 | 605 | QVQLVESGGGVVQPG | 704 | AIQLTQSPSSLSASVG |
| | | (EGFR) | | RSLRLSCAASGFTFSTY | | DRVTITCRASQDISSA |
| | | | | GMHWVRQAPGKGLE | | LVWYQQKPGKAPKL |
| | | | | WVAVIWDDGSYKYY | | LIYDASSLESGVPSRF |
| | | | | GDSVKGRFTISRDNSK | | SGSESGTDFTLTISSL |
| | | | | NTLYLQMNSLRAEDT | | QPEDFATYYCQQFNS |
| | | | | AVYYCARDGITMVRG | | YPLTFGGGTKVEIK |
| | | | | VMKDYFDYWGQGTL | | |
| | | | | VTVSS | | |
| IMC-11F8 | necitumumab | HER1 | 606 | QVQLQESGPGLVKPSQ | 705 | EIVMTQSPATLSLSPG |
| | | (EGFR) | | TLSLTCTVSGGSISSGD | | ERATLSCRASQSVSS |
| | | | | YYWSWIRQPPGKGLE | | YLAWYQQKPGQAPR |
| | | | | WIGYIYYSGSTDYNPS | | LLIYDASNRATGIPAR |
| | | | | LKSRVTMSVDTSKNQF | | FSGSGSGTDFTLTISS |
| | | | | SLKVNSVTAADTAVY | | LEPEDFAVYYCHQY |
| | | | | YCARVSIFGVGTFDY | | GSTPLTFGGGTKAEI |
| | | | | WGQGTLVTVSS | | K |
| MM-151 | PIX | HER1 | 607 | QVQLVQSGAEVKKPG | 706 | DIQMTQSPSTLSASV |
| | | (EGFR) | | SSVKVSCKASGGTFSS | | GDRVTITCRASQSISS |
| | | | | YAISWVRQAPGQGLE | | WWAWYQQKPGKAP |
| | | | | WMGSIIPIFGTVNYAQ | | KLLIYDASSLESGVPS |
| | | | | KFQGRVTITADESTST | | RFSGSGSGTEFTLTISS |
| | | | | AYMELSSLRSEDTAVY | | LQPDDFATYYCQQY |
| | | | | YCARDPSVNLYWYFD | | HAHPTTFGGGTKVEI |
| | | | | LWGRGTLVTVSS | | K |
| MM-151 | P2X | HER1 | 608 | QVQLVQSGAEVKKPG | 707 | DIVMTQSPDSLAVSL |
| | | (EGFR) | | SSVKVSCKASGGTFGS | | GERATINCKSSQSVL |
| | | | | YAISWVRQAPGQGLE | | YSPNNKNYLAWYQQ |
| | | | | WMGSIIPIFGAANPAQ | | KPGQPPKLLIYWAST |
| | | | | KSQGRVTITADESTST | | RESGVPDRFSGSGSG |
| | | | | AYMELSSLRSEDTAVY | | TDFTLTISSLQAEDVA |
| | | | | YCAKMGRGKVAFDI | | VYYCQQYYGSPITFG |
| | | | | WGQGTMVTVSS | | GGTKVEIK |
| MM-151 | P3X | HER1 | 609 | QVQLVQSGAEVKKPG | 708 | EIVMTQSPATLSVSPG |
| | | (EGFR) | | ASVKVSCKASGYAFTS | | ERATLSCRASQSVSS |
| | | | | YGINWVRQAPGQGLE | | NLAWYQQKPGQAPR |
| | | | | WMGWISAYNGNTYY | | LLIYGASTRATGIPAR |
| | | | | AQKLRGRVTMTTDTS | | FSGSGSGTEFTLTISSL |
| | | | | TSTAYMELRSLRSDDT | | QSEDFAVYYCQDYR |
| | | | | AVYYCARDLGGYGSG | | TWPRRVFGGGTKVE |
| | | | | SVPFDPWGQGTLVTVSS | | IK |
| TheraCIM | nimotuzumab | HER1 | 610 | QVQLQQSGAEVKKPG | 709 | DIQMTQSPSSLSASVG |
| | | (EGFR) | | SSVKVSCKASGYTFTN | | DRVTITCRSSQNIVHS |
| | | | | YYIYWVRQAPGQGLE | | NGNTYLDWYQQTPG |
| | | | | WIGGINPTSGGSNFNE | | KAPKLLIYKVSNRFS |
| | | | | KFKTRVTITADESSTT | | GVPSRFSGSGSGTDFT |
| | | | | AYMELSSLRSEDTAFY | | FTISSLQPEDIATYYC |
| | | | | FCTRQGLWFDSDGRG | | FQYSHVPWTFGQGT |
| | | | | FDFWGQGTTVTVSS | | KLQIT |
| Vectibix™ | panitumimab | HER1 | 611 | QVQLQESGPGLVKPSE | 710 | DIQMTQSPSSLSASVG |
| | | (EGFR) | | TLSLTCTVSGGSVSSG | | DRVTITCQASQDISN |
| | | | | DYYWTWIRQSPGKGL | | YLNWYQQKPGKAPK |
| | | | | EWIGHIYYSGNTNYNP | | LLIYDASNLETGVPSR |
| | | | | SLKSRLTISIDTSKTQFS | | FSGSGSGTDFTFTISSL |
| | | | | LKLSSVTAADTAIYYC | | QPEDIATYFCQHFDH |
| | | | | VRDRVTGAFDIWGQG | | LPLAFGGGTKVEIK |
| | | | | TMVTVSS | | |
| 07D06 | | HER1 | 612 | QIQLVQSGPELKKPGE | 711 | DVVMTQTPLSLPVSL |
| | | (EGFR) | | TVKISCKASGYTFTEY | | GDQASISCRSSQSLV |
| | | | | PIHWVKQAPGKGFKW | | HSNGNTYLHWYLQK |
| | | | | MGMIYTDIGKPTYAE | | PGQSPKLLIYKVSNR |
| | | | | EFKGRFAFSLETSASTA | | FSGVPDRFSGSGSGT |
| | | | | YLQINNLKNEDTATYF | | DFTLKISRVEAEDLG |
| | | | | CVRDRYDSLFDYWGQ | | VYFCSQSTHVPWTF |
| | | | | GTTLTVSS | | GGGTKLEIK |
| 12D03 | | HER1 | 613 | EMQLVESGGGFVKPG | 712 | DVVMTQTPLSLPVSL |
| | | (EGFR) | | GSLKLSCAASGFAFSH | | GDQASISCRSSQSLV |
| | | | | YDMSWVRQTPKQRLE | | HSNGNTYLHWYLQK |
| | | | | WVAYIASGGDITYYA | | PGQSPKLLIYKVSNR |
| | | | | DTVKGRFTISRDNAQN | | FSGVPDRFSGSGSGT |
| | | | | TLYLQMSSLKSEDTAM | | DFTLKISRVEAEDLG |
| | | | | FYCSRSSYGNNGDAL | | VYFCSQSTHVLTFGS |
| | | | | DFWGQGTSVTVSS | | GTKLEIK |
| | C1 | HER2 | 614 | QVQLVESGGGLVQPG | 713 | QSPSFLSAFVGDRITIT |
| | | | | GSLRLSCAASGFTFSSY | | CRASPGIRNYLAWY |
| | | | | AMGWVRQAPGKGLE | | QQKPGKAPKLLIYAA |
| | | | | WVSSISGSSRYIYYAD | | STLQSGVPSRFSGSGS |
| | | | | SVKGRFTISRDNSKNT | | GTDFTLTISSLQPEDF |
| | | | | LYLQMNSLRAEDTAV | | ATYYCQQYNSYPLSF |
| | | | | YYCAKMDASGSYFNF | | GGGTKVEIK |
| | | | | WGQGTLVTVSS | | |
| Erbicin | | HER2 | 615 | QVQLLQSAAEVKKPGE | 714 | QAVVTQEPSFSVSPG |
| | | | | SLKISCKGSGYSFTSY | | GTVTLTCGLSSGSVS |
| | | | | WIGWVRQMPGKGLE | | TSYYPSWYQQTPGQ |
| | | | | WMGIIYPGDSDTRYSP | | APRTLIYSTNTRSSGV |
| | | | | SFQGQVTISADKSISTA | | PDRFSGSILGNKAALT |
| | | | | YLQWSSLKASDTAVY | | ITGAQADDESDYYCV |
| | | | | YCARWRDSPLWGQGT | | LYMGSGQYVFGGGT |
| | | | | LVTVSS | | KLTVL |
| Herceptin | trastuzumab | HER2 | 616 | EVQLVESGGGLVQPGG | 715 | DIQMTQSPSSLSASVG |
| | | | | SLRLSCAASGFNIKDT | | DRVTITCRASQDVNT |
| | | | | YIHWVRQAPGKGLEW | | AVAWYQQKPGKAPK |
| | | | | VARIYPTNGYTRYADS | | LLIYSASFLYSGVPSR |
| | | | | VKGRFTISADTSKNTA | | FSGSRSGTDFTLTISSL |
| | | | | YLQMNSLRAEDTAVY | | QPEDFATYYCQQHY |
| | | | | YCSRWGGDGFYAMD | | TTPPTFGQGTKVEIK |
| | | | | YWGQGTLVTVSS | | |
| MAGH22 | margetuximab | HER2 | 617 | QVQLQQSGPELVKPGA | 716 | DIVMTQSHKFMSTSV |
| | | | | SLKLSCTASGFNIKDT | | GDRVSITCKASQDVN |
| | | | | YIHWVKQRPEQGLEWI | | TAVAWYQQKPGHSP |
| | | | | GRIYPTNGYTRYDPKF | | KLLIYSASFRYTGVPD |
| | | | | QDKATITADTSSNTAY | | RFTGSRSGTDFTFTIS |
| | | | | LQVSRLTSEDTAVYYC | | SVQAEDLAVYYCQQ |
| | | | | SRWGGDGFYAMDYW | | HYTTPPTFGGGTKVE |
| | | | | GQGASVTVSS | | IK |
| MM-302 | F5 | HER2 | 618 | QVQLVESGGGLVQPG | 717 | QSVLTQPPSVSGAPG |
| | | | | GSLRLSCAASGFTFRSY | | QRVTISCTGSSSNIGA |
| | | | | AMSWVRQAPGKGLE | | GYGVHWYQQLPGTA |
| | | | | WVSAISGRGDNTYYA | | PKLLIYGNTNRPSGV |
| | | | | DSVKGRFTISRDNSKN | | PDRFSGFKSGTSASLA |
| | | | | TLYLQMNSLRAEDTA | | ITGLQAEDEADYYCQ |
| | | | | VYYCAKMTSNAFAFD | | FYDSSLSGWVFGGG |
| | | | | YWGQGTLVTVSS | | TKLTVL |
| Perjeta | pertuzumab | HER2 | 619 | EVQLVESGGGLVQPGG | 718 | DIQMTQSPSSLSASVG |
| | | | | SLRLSCAASGFTFTDY | | DRVTITCKASQDVSI |
| | | | | TMDWVRQAPGKGLE | | GVAWYQQKPGKAPK |
| | | | | WVADVNPNSGGSIYN | | LLIYSASYRYTGVPSR |
| | | | | QRFKGRFTLSVDRSKN | | FSGSGSGTDFTLTISS |
| | | | | TLYLQMNSLRAEDTA | | LQPEDFATYYCQQY |
| | | | | VYYCARNLGPSFYFD | | YIYPYTFGQGTKVEI |
| | | | | YWGQGTLVTVSS | | K |
| MM-121/ | | HER3 | 620 | EVQLLESGGGLVQPGG | 719 | QSALTQPASVSGSPG |
| SAR2562 | | | | SLRLSCAASGFTFSHY | | QSITISCTGTSSDVGS |
| 12 | | | | VMAWVRQAPGKGLE | | YNVVSWYQQHPGKA |
| | | | | WVSSISSSGGWTLYA | | PKLIIYEVSQRPSGVS |
| | | | | DSVKGRFTISRDNSKN | | NRFSGSKSGNTASLTI |
| | | | | TLYLQMNSLRAEDTA | | SGLQTEDEADYYCCS |
| | | | | VYYCTRGLKMATIFD | | YAGSSIFVIFGGGTK |
| | | | | YWGQGTLVTVSS | | VTVL |
| MEHD79 | Duligotumab | HER1 | 621 | EVQLVESGGGLVQPGG | 720 | DIQMTQSPSSLSASVG |
| 45A | | (EGFR)/ | | SLRLSCAASGFTLSGD | | DRVTITCRASQNIAT |
| | | HER3 | | WIHWVRQAPGKGLE | | DVAWYQQKPGKAPK |
| | | | | WVGEISAAGGYTDYA | | LLIYSASFLYSGVPSR |
| | | | | DSVKGRFTISADTSKN | | FSGSGSGTDFTLTISS |
| | | | | TAYLQMNSLRAEDTA | | LQPEDFATYYCQQSE |
| | | | | VYYCARESRVSFEAA | | PEPYTFGQGTKVEIK |
| | | | | MDYWGQGTLVTVSS | | |
| MM-111 | | HER2/ | 622 | QVQLQESGGGLVKPG | 721 | QSALTQPASVSGSPG |
| | | 3 | | GSLRLSCAASGFTFSSY | | QSITISCTGTSSDVGG |
| | | | | WMSWVRQAPGKGLE | | YNFVSWYQQHPGKA |
| | | | | WVANINRDGSASYYV | | PKLMIYDVSDRPSGV |
| | | | | DSVKGRFTISRDDAKN | | SDRFSGSKSGNTASLI |
| | | | | SLYLQMNSLRAEDTAV | | ISGLQADDEADYYCS |
| | | | | YYCARDRGVGYFDL | | SYGSSSTHVIFGGGT |
| | | | | WGRGTLVTVSS | | KVTVL |
| MM-111 | | HER2/ | 623 | QVQLVQSGAEVKKPG | 722 | QSVLTQPPSVSAAPGQ |
| | | 3 | | ESLKISCKGSGYSFTSY | | KVTISCSGSSSNIGNN |
| | | | | WIAWVRQMPGKGLEY | | YVSWYQQLPGTAPK |
| | | | | MGLIYPGDSDTKYSPS | | LLIYDHTNRPAGVPD |
| | | | | FQGQVTISVDKSVSTA | | RFSGSKSGTSASLAIS |
| | | | | YLQWSSLKPSDSAVYF | | GFRSEDEADYYCAS |
| | | | | CARHDVGYCTDRTCA | | WDYTLSGWVFGGG |
| | | | | KWPEWLGVWGQGTL | | TKLTVL |
| | | | | VTVSS | | |
| | Hu3S193 | Lewis- | 624 | EVQLVESGGGVVQPG | 723 | DIQMTQSPSSLSASVG |
| | | Y | | RSLRLSCSTSGFTFSDY | | DRVTITCRSSQRIVHS |
| | | | | YMYWVRQAPGKGLE | | NGNTYLEWYQQTPG |
| | | | | WVAYMSNVGAITDYP | | KAPKLLIYKVSNRFS |
| | | | | DTVKGRFTISRDNSKN | | GVPSRFSGSGSGTDFT |
| | | | | TLFLQMDSLRPEDTGV | | FTISSLQPEDIATYYC |
| | | | | YFCARGTRDGSWFAY | | FQGSHVPFTFGQGT |
| | | | | WGQGTPVTVSS | | KLQIT |
| BAY 94- | anetumab | Mesothelin | 625 | QVELVQSGAEVKKPGE | 724 | DIALTQPASVSGSPGQ |
| 9343 | ravtansine | | | SLKISCKGSGYSFTSY | | SITISCTGTSSDIGGY |
| | | | | WIGWVRQAPGKGLEW | | NSVSWYQQHPGKAP |
| | | | | MGIIDPGDSRTRYSPSF | | KLMIYGVNNRPSGVS |
| | | | | QGQVTISADKSISTAYL | | NRFSGSKSGNTASLTI |
| | | | | QWSSLKASDTAMYYC | | SGLQAEDEADYYCSS |
| | | | | ARGQLYGGTYMDGW | | YDIESATPVFGGGTK |
| | | | | GQGTLVTVSS | | LTVL |
| | SS1 | Mesothelin | 626 | QVQLQQSGPELEKPGA | 725 | DIELTQSPAIMSASPG |
| | | | | SVKISCKASGYSFTGYT | | EKVTMTCSASSSVSY |
| | | | | MNWVKQSHGKSLEWI | | MHWYQQKSGTSPKR |
| | | | | GLITPYNGASSYNQKF | | WIYDTSKLASGVPGR |
| | | | | RGKATLTVDKSSSTAY | | FSGSGSGNSYSLTISS |
| | | | | MDLLSLTSEDSAVYFC | | VEAEDDATYYCQQW |
| | | | | ARGGYDGRGFDYWGQ | | SGYPLTFGAGTKLEIK |
| | | | | GTTVTVSS | | |
| | | Mesothelin | 627 | QVYLVESGGGVVQPG | 726 | EIVLTQSPATLSLSPG |
| | | | | RSLRLSCAASGITFSIY | | ERATLSCRASQSVSS |
| | | | | GMHWVRQAPGKGLE | | YLAWYQQKPGQAPR |
| | | | | WVAVIWYDGSHEYY | | LLIYDASNRATGIPAR |
| | | | | ADSVKGRFTISRDNSK | | FSGSGSGTDFTLTISS7 |
| | | | | NTLYLLMNSLRAED | | LEPEDFAVYYCQQ |
| | | | | TAVYYCARDGDYYDS | | RSNWPLTFGGGTKV |
| | | | | GSPLDYWGQGTLVTV | | EIK |
| | | | | SS | | |
| | | Mesothelin | 628 | QVHLVESGGGVVQPG | 727 | EIVLTQSPATLSLSPG |
| | | | | RSLRLSCVASGITFRIY | | ERATLSCRASQSVSS |
| | | | | GMHWVRQAPGKGLE | | YLAWYQQKPGQAPR |
| | | | | WVAVLWYDGSHEYY | | LLIYDASNRATGIPAR |
| | | | | ADSVKGRFTISRDNSK | | FSGSGSGTDFTLTISS |
| | | | | NTLYLQMNSLRAED | | LEPEDFAVYYCQQ |
| | | | | TAIYYCARDGDYYDS | | RSNWPLTFGGGTKV |
| | | | | GSPLDYWGQGTLVTV | | EIK |
| | | | | SS | | |
| | | Mesothelin | 629 | EVHLVESGGGLVQPGG | 728 | EIVLTQSPGTLSLSPG |
| | | | | SLRLSCAASGFTFSRY | | ERATLSCRASQSVSS |
| | | | | WMSWVRQAQGKGLE | | SYLAWYQQKPGQAP |
| | | | | WVASIKQAGSEKTYV | | RLLIYGASSRATGIPD |
| | | | | DSVKGRFTISRDNAKN | | RFSGSGSGTDFTLTIS |
| | | | | SLSLQMNSLRAED | | RLEPEDFAVYYCQ |
| | | | | TAVYYCAREGAYYYD | | QYGSSQYTFGQGTK |
| | | | | SASYYPYYYYYSMDV | | LEIK |
| | | | | WGQGTTVTVSS | | |
| MORAb- | amatuximab | Mesothelin | 630 | QVQLQQSGPELEKPGA | 729 | DIELTQSPAIMSASPG |
| 009 | | | | SVKISCKASGYSFTGY | | EKVTMTCSASSSVSY |
| | | | | TMNWVKQSHGKSLE | | MHWYQQKSGTSPKR |
| | | | | WIGLITPYNGASSYNQ | | WIYDTSKLASGVPGR |
| | | | | KFRGKATLTVDKSSST | | FSGSGSGNSYSLTISS |
| | | | | AYMDLLSLTSEDSAVY | | VEAEDDATYYCQQW |
| | | | | FCARGGYDGRGFDY | | SKHPLTFGSGTKVEI |
| | | | | WGSGTPVTVSS | | K |
| hPAM4 | | MUC- | 631 | EVQLQESGPELVKPGA | 730 | DIVMTQSPAIMSASP |
| | | 1 | | SVKMSCKASGYTFPSY | | GEKVTMTCSASSSVS |
| | | | | VLHWVKQKPGQGLE | | SSYLYWYQQKPGSSP |
| | | | | WIGYINPYNDGTQYN | | KLWIYSTSNLASGVP |
| | | | | EKFKGKATLTSDKSSS | | ARFSGSGSGTSYSLTI |
| | | | | TAYMELSRLTSED | | SSMEAEDAASYFCH |
| | | | | SAVYYCARGFGGSYG | | QWNRYPYTFGGGTK |
| | | | | FAYWGQGTLITVSA | | LEIK |
| hPAM4- | clivatuzumab | MUC1 | 632 | QVQLQQSGAEVKKFG | 731 | DIQLTQSPSSLSASVG |
| Cide | | | | ASVKVSCEASGYTFPS | | DRVTMTCSASSSVSS |
| | | | | YVLHWVKQAPGQGLE | | SYLYWYQQKPGKAP |
| | | | | WIGYINPYNDGTQTN | | KLWIYSTSNLASGVP |
| | | | | KKFKGKATLTRDTSIN | | ARFSGSGSGTDFTLTI |
| | | | | TAYMELSRLRSDDTAV | | SSLQPEDSASYFCHQ |
| | | | | YYCARGFGGSYGFAY | | WNRYPYTFGGGTRL |
| | | | | NGQGTLVTVSS | | EIK |
| SAR5666 | huDS6v1.01 | MUC1 | 754 | QAQLQVSGAEVVKPG | 732 | EIVLTQSPATMSASPG |
| 58 | | | | ASVKMSCKASGYTFTS | | ERVTITCSAHSSVSF |
| | | | | YNMHWVKQTPGQGL | | MHWFQQKPGTSPKL |
| | | | | EWIGYIYPGNGATNY | | WIYSTSSLASGVPAR |
| | | | | NQKFQGKATLTADTS | | FGGSGSGTSYSLTISS |
| | | | | SSTAYMQISSLTSEDSA | | MEAEDAATYYCQQR |
| | | | | VYFCARGDSVPFAYW | | SSFPLTFGAGTKLEL |
| | | | | GQGTLVTVSA | | K |
| Theragyn | Pemtumomab | MUC1 | 633 | QVQLQQSGAELMKPG | 733 | DIVMSQSPSSLAVSV |
| | muHMFG1 | | | ASVKISCKATGYTFSA | | GEKVTMSCKSSQSLL |
| | | | | YWIEWVKQRPGHGLE | | YSSNQKIYLAWYQQ |
| | | | | WIGEILPGSNNSRYNE | | KPGQSPKLLIYWAST |
| | | | | KFKGKATFTADTSSNT | | RESGVPDRFTGGGSG |
| | | | | AYMQLSSLTSEDSAVY | | TDFTLTISSVKAEDLA |
| | | | | YCSRSYDFAWFAYWG | | VYYCQQYYRYPRTF |
| | | | | QGTPVTVSA | | GGGTKLEIK |
| Therex | Sontuzumab | MUC1 | 634 | QVQLVQSGAEVKKPG | 734 | DIQMTQSPSSLSASVG |
| | huHMFG1 | | | ASVKVSCKASGYTFSA | | DRVTITCKSSQSLLY |
| | AS1402 | | | YWIEWVRQAPGKGLE | | SSNQKIYLAWYQQK |
| | R1150 | | | WVGEILPGSNNSRYN | | PGKAPKLLIYWASTR |
| | | | | EKFKGRVTVTRDTST | | ESGVPSRFSGSGSGT |
| | | | | NTAYMELSSLRSEDTA | | DFTFTISSLQPEDIATY |
| | | | | VYYCARSYDFAWFAY | | YCQQYYRYPRTFGQ |
| | | | | WGQGTLVTVSS | | GTKVEIK |
| MDX- | | PD-L1 | 635 | QVQLVQSGAEVKKPG | 735 | EIVLTQSPATLSLSPG |
| 1105 or | | | | SSVKVSCKTSGDTFST | | ERATLSCRASQSVSS |
| BMS- | | | | YAISWVRQAPGQGLE | | YLAWYQQKPGQAPR |
| 936559 | | | | WMGGIIPIFGKAHYA | | LLIYDASNRATGIPAR |
| | | | | QKFQGRVTITADESTS | | FSGSGSGTDFTLTISS |
| | | | | TAYMELSSLRSEDTAV | | LEPEDFAVYYCQQRS |
| | | | | YFCARKFHFVSGSPFG | | NWPTFGQGTKVEIK |
| | | | | MDVWGQGTTVTVSS | | |
| MEDI- | durvalumab | PD-L1 | 636 | EVQLVESGGGLVQPGG | 736 | EIVLTQSPGTLSLSPG |
| 4736 | | | | SLRLSCAASGFTFSRY | | ERATLSCRASQRVSS |
| | | | | WMSWVRQAPGKGLE | | SYLAWYQQKPGQAP |
| | | | | WVANIKQDGSEKYYV | | RLLIYDASSRATGIPD |
| | | | | DSVKGRFTISRDNAKN | | RFSGSGSGTDFTLTIS |
| | | | | SLYLQMNSLRAEDTAV | | RLEPEDFAVYYCQQ |
| | | | | YYCAREGGWFGELA | | YGSLPWTFGQGTKV |
| | | | | FDYWGQGTLVTVSS | | EIK |
| MPDL328 | atezolizumab | PD-L1 | 637 | EVQLVESGGGLVQPGG | 737 | DIQMTQSPSSLSASVG |
| 0A | | | | SLRLSCAASGFTFSDS | | DRVTITCRASQDVST |
| | | | | WIHWVRQAPGKGLEW | | AVAWYQQKPGKAPK |
| | | | | VAWISPYGGSTYYAD | | LLIYSASFLYSGVPSR |
| | | | | SVKGRFTISADTSKNT | | FSGSGSGTDFTLTISS |
| | | | | AYLQMNSLRAEDTAV | | LQPEDFATYYCQQY |
| | | | | YYCARRHWPGGFDY | | LYHPATFGQGTKVEI |
| | | | | WGQGTLVTVSS | | K |
| MSB0010 | avelumab | PD-L1 | 638 | EVQLLESGGGLVQPGG | 738 | QSALTQPASVSGSPG |
| 718C | | | | SLRLSCAASGFTFSSYI | | QSITISCTGTSSDVGG |
| | | | | MMWVRQAPGKGLEW | | YNYVSWYQQHPGKA |
| | | | | VSSIYPSGGITFYADTV | | PKLMIYDVSNRPSGV |
| | | | | KGRFTISRDNSKNTLY | | SNRFSGSKSGNTASL |
| | | | | LQMNSLRAEDTAVYY | | TISGLQAEDEADYYC |
| | | | | CARIKLGTVTTVDYW | | SSYTSSSTRVFGTGT |
| | | | | GQGTLVTVSS | | KVTVL |
| MLN591 | | PSMA | 639 | EVQLVQSGPEVKKPGA | 739 | DIQMTQSPSSLSTSVG |
| | | | | TVKISCKTSGYTFTEY | | DRVTLTCKASQDVG |
| | | | | TIHWVKQAPGKGLEW | | TAVDWYQQKPGPSP |
| | | | | IGNINPNNGGTTYNQ | | KLLIYWASTRHTGIP |
| | | | | KFEDKATLTVDKSTDT | | SRFSGSGSGTDFTLTI |
| | | | | AYMELSSLRSEDTAVY | | SSLQPEDFADYYCQQ |
| | | | | YCAAGWNFDYWGQG | | YNSYPLTFGPGTKVD |
| | | | | TLLTVSS | | IK |
| MT112 | pasotuxizumab | PSMA | 640 | QVQLVESGGGLVKPGE | 740 | DIQMTQSPSSLSASVG |
| | | | | SLRLSCAASGFTFSDY | | DRVTITCKASQNVDT |
| | | | | YMYWVRQAPGKGLE | | NVAWYQQKPGQAPK |
| | | | | WVAIISDGGYYTYYSD | | SLIYSASYRYSDVPSR |
| | | | | IIKGRFTISRDNAKNSL | | FSGSASGTDFTLTISS |
| | | | | YLQMNSLKAEDTAVY | | VQSEDFATYYCQQY |
| | | | | YCARGFPLLRHGAM | | DSYPYTFGGGTKLEI |
| | | | | DYWGQGTLVTVSS | | K |
| | | ROR1 | 641 | QEQLVESGGRLVTPGG | 741 | ELVLTQSPSVSAALG |
| | | | | SLTLSCKASGFDFSAY | | SPAKITCTLSSAHKT |
| | | | | YMSWVRQAPGKGLE | | DTIDWYQQLQGEAP |
| | | | | WIATIYPSSGKTYYAT | | RYLMQVQSDGSYTK |
| | | | | WVNGRFTISSDNAQNT | | RPGVPDRFSGSSSGA |
| | | | | VDLQMNSLTAAD | | DRYLIIPSVQADDEA |
| | | | | RATYFCARDSYADDG | | DY |
| | | | | ALFNIWGPGTLVTISS | | YCGADYIGGYVFGG |
| | | | | | | GTQLTVTG |
| | | ROR1 | 642 | EVKLVESGGGLVKPGG | 742 | DIKMTQSPSSMYASL |
| | | | | SLKLSCAASGFTFSSYA | | GERVTITCKASPDINS |
| | | | | MSWVRQIPEKRLEWV | | YLSWFQQKPGKSPKT |
| | | | | ASISRGGTTYYPDSVK | | LIYRANRLVDGVPSR |
| | | | | GRFTISRDNVRNILYLQ | | FSGGGSGQDYSLTINS |
| | | | | MSSLRSEDT | | LEYEDMGIYYCLQ |
| | | | | AMYYCGRYDYDGYY | | YDEFPYTFGGGTKLE |
| | | | | AMDYWGQGTSVTVSS | | MK |
| | | ROR1 | 643 | QSLEESGGRLVTPGTPL | 743 | ELVMTQTPSSVSAAV |
| | | | | TLTCTVSGIDLNSHWM | | GGTVTINCQASQSIG |
| | | | | SWVRQAPGKGLEWIGI | | SYLAWYQQKPGQPP |
| | | | | IAASGSTYYANWAKG | | KLLIYYASNLASGVP |
| | | | | RFTISKTSTTVDLRIASP | | SRFSGSGSGTEYTLTI |
| | | | | TTEDTATY | | SGVQREDAATYYCLG |
| | | | | FCARDYGDYRLVTFNI | | SLSNSDNVFGGGTEL |
| | | | | WGPGTLVTVSS | | EIL |
| | | ROR1 | 644 | QSVKESEGDLVTPAGN | 744 | ELVMTQTPSSTSGAV |
| | | | | LTLTCTASGSDINDYPI | | GGTVTINCQASQSID |
| | | | | SWVRQAPGKGLEWIG | | SNLAWFQQKPGQPPT |
| | | | | FINSGGSTWYASWVK | | LLIYRASNLASGVPS |
| | | | | GRFTISRTSTTVDLKM | | RFSGSRSGTEYTLTIS |
| | | | | TSLTTDDTATY | | GVQREDAATYYCLG |
| | | | | FCARGYSTYYCDFNI | | GVGNVSYRTSFGGG |
| | | | | WGPGTLVTISS | | TEVVVK |
| CC49 | | TAG- | 645 | QVQLVQSGAEVVKPG | 745 | DIVMSQSPDSLAVSL |
| (Humanized) | | 72 | | ASVKISCKASGYTFTD | | GERVTLNCKSSQSLL |
| | | | | HAIHWVKQNPGQRLE | | YSGNQKNYLAWYQ |
| | | | | WIGYFSPGNDDFKYN | | QKPGQSPKLLIYWAS |
| | | | | ERFKGKATLTADTSAS | | ARESGVPDRFSGSGS |
| | | | | TAYVELSSLRSEDTAV | | GTDFTLTISSVQAEDV |
| | | | | YFCTRSLNMAYWGQG | | AVYYCQQYYSYPLT |
| | | | | TLVTVSS | | FGAGTKLELK |
| | Murine A1 | TPBG/ | 646 | QIQLVQSGPELKKPGE | 746 | SIVMTQTPKFLLVSA |
| | | 5T4 | | TVKISCKASGYTFTNF | | GDRVTITCKASQSVS |
| | | | | GMNWVKQGPGEGLK | | NDVAWYQQKPGQSP |
| | | | | WMGWINTNTGEPRY | | KLLINFATNRYTGVP |
| | | | | AEEFKGRXAFSLETTA | | NRFTGSGYGTDFTFTI |
| | | | | STAYLQINNLKNEDTA | | STVQAEDLALYFCQQ |
| | | | | TYFCARDWDGAYFFD | | DYSSPWTFGGGTKLE |
| | | | | YWGQGTTLTVSS | | IK |
| | Murine A2 | TPBG/ | 647 | QVQLQQSRPELVKPGA | 747 | SVIMSRGQIVLTQSPA |
| | | 5T4 | | SVKMSCKASGYTFTD | | IMSASLGERVTLTCT |
| | | | | YVISWVKQRTGQGLE | | ASSSVNSNYLHWYQ |
| | | | | WIGEIYPGSNSIYYNE | | QKPGSSPKLWIYSTS |
| | | | | KFKGRATLTA | | NLASGVPARFSGSGS |
| | | | | DKSSSTAYMQLSSLTS | | GTSYSLTISSMEAEDA |
| | | | | EDSAVYFCAMGGNYG | | ATYYCHQYHRSPLT |
| | | | | FDYWGQGTTLTVSS | | FGAGTKLELK |
| | Murine A3 | TPBG/ | 648 | EVQLVESGGGLVQPKG | 748 | DIVMTQSHIFMSTSV |
| | | 5T4 | | SLKLSCAASGFTFNTY | | GDRVSITCKASQDVD |
| | | | | AMNWVRQAPGKGLE | | TAVAWYQQKPGQSP |
| | | | | WVARIRSKSNNYATY | | KLLIYWASTRLTGVP |
| | | | | YADSVKDRFTISRDDS | | DRFTGSGSGTDFTLTI |
| | | | | QSMLYLQMNNLKTED | | SNVQSEDLADYFCQQ |
| | | | | TAMYXCVRQWDYDV | | YSSYPYTFGGGTKLE |
| | | | | RAMNYWGQGTSVTVSS | | IK |
| IMMU- | hRS-7 | TROP- | 649 | QVQLQQSGSELKKPGA | 749 | DIQLTQSPSSLSASVG |
| 132 | | 2 | | SVKVSCKASGYTFTNY | | DRVSITCKASQDVSI |
| | | | | GMNWVKQAPGQGLK | | AVAWYQQKPGKAPK |
| | | | | WMGWINTYTGEPTY | | LLIYSASYRYTGVPD |
| | | | | TDDFKGRFAFSLDTSV | | RFSGSGSGTDFTLTIS |
| | | | | STAYLQISSLKADDTA | | SLQPEDFAVYYCQQ |
| | | | | VYFCARGGFGSSYWY | | HYITPLTFGAGTKVE |
| | | | | FDVWGQGSLVTVSS | | IK |
| IMC-18F1 | icrucumab | VEGF | 650 | QAQVVESGGGVVQSG | 750 | EIVLTQSPGTLSLSPG |
| | | RI | | RSLRLSCAASGFAFSS | | ERATLSCRASQSVSS |
| | | | | YGMHWVRQAPGKGL | | SYLAWYQQKPGQAP |
| | | | | EWVAVIWYDGSNKY | | RLLIYGASSRATGIPD |
| | | | | YADSVRGRFTISRDNS | | RFSGSGSGTDFTLTIS |
| | | | | ENTLYLQMNSLRAEDT | | RLEPEDFAVYYCQQ |
| | | | | AVYYCARDHYGSGVH | | YGSSPLTFGGGTKVE |
| | | | | HYFYYGLDVWGQGTT | | IK |
| | | | | VTVSS | | |
| Cyramza | ramucirumab | VEGF | 651 | EVQLVQSGGGLVKPG | 751 | DIQMTQSPSSVSASIG |
| | | R2 | | GSLRLSCAASGFTFSS | | DRVTITCRASQGIDN |
| | | | | YSMNWVRQAPGKGLE | | WLGWYQQKPGKAPK |
| | | | | WVSSISSSSSYIYYADS | | LLIYDASNLDTGVPS |
| | | | | VKGRFTISRDNAKNSL | | RFSGSGSGTYFTLTIS |
| | | | | YLQMNSLRAEDTAVY | | SLQAEDFAVYFCQQ |
| | | | | YCARVTDAFDIWGQG | | AKAFPPTFGGGTKV |
| | | | | TMVTVSSA | | DIK |
| g165DFM- | alacizumabpegol | VEGF | 652 | EVQLVESGGGLVQPGG | 752 | DIQMTQSPSSLSASVG |
| PEG | | R2 | | SLRLSCAASGFTFSSY | | DRVTITCRASQDIAG |
| | | | | GMSWVRQAPGKGLE | | SLNWLQQKPGKAIKR |
| | | | | WVATITSGGSYTYYV | | LIYATSSLDSGVPKRF |
| | | | | DSVKGRFTISRDNAKN | | SGSRSGSDYTLTISSL |
| | | | | TLYLQMNSLRAEDTA | | QPEDFATYYCLQYGS |
| | | | | VYYCVRIGEDALDYW | | FPPTFGQGTKVEIK |
| | | | | GQGTLVTVSS | | |
| Imclone6.64 | | VEGF | 653 | KVQLQQSGTELVKPGA | 753 | DIVLTQSPASLAVSLG |
| | | R2 | | SVKVSCKASGYIFTEYI | | QRATISCRASESVDSY |
| | | | | IHWVKQRSGQGLEWIG | | GNSFMHWYQQKPGQ |
| | | | | WLYPESNIIKYNEKFK | | PPKLLIYRASNLESGI |
| | | | | DKATLTADKSSSTVYM | | PARFSGSGSRTDFTLT |
| | | | | ELSRLTSEDSAVYFCTR | | INPVEADDVATYYCQ |
| | | | | HDGTNFDYWGQGTTL | | QSNEDPLTFGAGTKL |
| | | | | TVSSA | | ELK |

*underlined & bolded sequences, if present, are CDRs within the VL and VH

### Anti-Epcam (Epithelial Cell Adhesion Molecule) Binding Domains:

In some embodiments of the compositions of this disclosure, the binding domain can have specific binding affinity to the tumor-specific marker EpCAM. The binding domain can comprise VL and VH derived from a monoclonal antibody to EpCAM. Some embodiments of the compositions of this disclosure can comprise a bispecific bioactive assembly comprising the binding domain specific for EpCAM and another binding domain (e.g., having specific binding affinity to an effector cell).

Monoclonal antibodies to EpCAM are known in the art (such as described more fully in the following paragraphs). Exemplary, non-limiting examples of EpCAM monoclonal antibodies and the VL and VH sequences thereof are presented in Table 6. Some embodiments of the binding domain with binding affinity to the tumor-specific marker EpCAM can comprise anti-EpCAM VL and VH sequences set forth in Table 6. Some embodiments of the binding domain with binding affinity to the tumor-specific marker EpCAM can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequences of the anti-EpCAM antibodies (such as 4D5MUCB) of Table 6. Some embodiments of the binding domain with binding affinity to the tumor-specific marker EpCAM can comprise the CDR-L1 region, the CDR-L2 region, the CDR-L3 region, the CDR-H1 region, the CDR-H2 region, and the CDR-H3 region, wherein each can be derived from the respective VL and VH sequences set forth in Table 6. Some embodiments of the compositions of this disclosure can comprise a bispecific bioactive assembly comprising a binding domain specific for EpCAM and another binding domain (e.g., having specific binding affinity to an effector cell). In some embodiments of the compositions of this disclosure, the binding domain specific for EpCAM can have a K_{d} value of greater than 10⁻⁷ to 10⁻¹⁰ M, as determined using an in vitro binding assay. The binding domains can be in a scFv format. The binding domains can be in a single chain diabody format.

In general, epithelial cell adhesion molecule (EpCAM, also known as 17-1A antigen) is a 40-kDa membrane-integrated glycoprotein composed of 314 amino acids expressed in certain epithelia and on many human carcinomas (see, Balzar, The biology of the 17-1A antigen (Ep-CAM), J. Mol. Med. 1999, 77:699-712). EpCAM was initially discovered by use of the murine monoclonal antibody 17-1A/edrecolomab that was generated by immunization of mice with colon carcinoma cells (Goettlinger, Int J Cancer. 1986; 38, 47-53 and Simon, Proc. Natl. Acad. Sci. USA. 1990; 87, 2755-2759). Because of their epithelial cell origin, tumor cells from most carcinomas express EpCAM on their surface (more so than normal, healthy cells), including the majority of primary, metastatic, and disseminated non-small cell lung carcinoma cells (Passlick, B., et al. The 17-1A antigen is expressed on primary, metastatic and disseminated non-small cell lung carcinoma cells. Int. J. Cancer 87(4):548-552, 2000), gastric and gastro-oesophageal junction adenocarcinomas (Martin, I. G., Expression of the 17-1A antigen in gastric and gastro-oesophageal junction adenocarcinomas: a potential immunotherapeutic target? J Clin Pathol 1999; 52:701-704), and breast and colorectal cancer (Packeisen J, et al. Detection of surface antigen 17-1A in breast and colorectal cancer. Hybridoma. 1999 18(1):37-40) and, therefore, are an attractive target for immunotherapy approaches. Indeed, increased expression of EpCAM correlates to increased epithelial proliferation; in breast cancer, overexpression of EpCAM on tumor cells is a predictor of survival (Gastl, Lancet. 2000, 356, 1981-1982). Due to their epithelial cell origin, tumor cells from most carcinomas still express EpCAM on their surface, and the bispecific solitomab single-chain antibody composition that targets EpCAM on tumor cells and also contains a CD3 binding region has been proposed for use against primary uterine and ovarian CS cell lines (Ferrari F, et al., Solitomab, an EpCAM/CD3 bispecific antibody construct (BITE®), is highly active against primary uterine and ovarian carcinosarcoma cell lines in vitro. J Exp Clin Cancer Res. 2015 34:123). Monoclonal antibodies to EpCAM are known in the art. The EpCAM monclonals ING-1, 3622W94, adecatumumab and edrecolomab have been described as having been tested in human patients (Münz, M. Side-by-side analysis of five clinically tested anti-EpCAM monoclonal antibodies Cancer Cell International, 10:44-56, 2010). Bispecific antibodies directed against EpCAM and against CD3 have also been described, including construction of two different bispecific antibodies by fusing a hybridoma producing monoclonal antibody against EpCAM with either of the two hybridomas OKT3 and 9.3 (Möller, SA, Reisfeld, RA, Bispecific-monoclonal-antibody-directed lysis of ovarian carcinoma cells by activated human T lymphocytes. Cancer Immunol. Immunother. 33:210-216, 1991). Other examples of bispecific antibodies against EpCAM include BiUII, (anti-CD3 (rat) x anti-EpCAM (mouse)) (Zeidler, J. Immunol., 1999, 163:1247-1252), a scFv CD3/17-1A-bispecific (Mack, M. A small bispecific antibody composition expressed as a functional single-chain molecule with high tumor cell cytotoxicity. Proc. Natl. Acad. Sci., 1995, 92:7021-7025), and a partially humanized bispecific diabody having anti-CD3 and antiEpCAM specificity (Helfrich, W. Construction and characterization of a bispecific diabody for retargeting T cells to human carcinomas. Int. J. Cancer, 1998, 76:232-239).

### Anti-CCR5 Binding Domains:

In some embodiments of the compositions of this disclosure, the binding domain can have specific binding affinity to the marker/antigen CCR5. Some embodiments of the compositions of this disclosure can comprise a bispecific bioactive assembly comprising the binding domain specific for CCR5 and another binding domain (e.g., having specific binding affinity to an effector cell). The binding domain can comprise VL and VH derived from a monoclonal antibody to CCR5. Monoclonal antibodies to CCR5 are known in the art. Some embodiments of the binding domain with binding affinity to the marker/antigen CCR5 can comprise anti-CCR5 VL and VH sequence(s). Some embodiments of the binding domain with binding affinity to the marker/antigen CCR5 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-CCR5 antibody/antibodies. Some embodiments of the binding domain with binding affinity to the marker/antigen CCR5 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for CCR5 can have a K_{d} value of greater than 10⁻⁷ to 10⁻¹⁰ M, as determined using an in vitro binding assay.

### Anti-CD19 Binding Domains:

In some embodiments of the compositions of this disclosure, the binding domain can have specific binding affinity to the marker/antigen CD19. Some embodiments of the compositions of this disclosure can comprise a bispecific bioactive assembly comprising the binding domain specific for CD19 and another binding domain (e.g., having specific binding affinity to an effector cell). The binding domain can comprise VL and VH derived from a monoclonal antibody to CD19. Monoclonal antibodies to CD19 are known in the art. Exemplary, non-limiting example(s) of CD19 monoclonal antibodies and the VL and VH sequences thereof are presented in Table 6. Some embodiments of the binding domain with binding affinity to the marker/antigen CD19 can comprise anti-CD19 VL and VH sequence(s) set forth in Table 6. Some embodiments of the binding domain with binding affinity to the marker/antigen CD19 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-CD19 antibody/antibodies (e.g., MT103) of Table 6. Some embodiments of the binding domain with binding affinity to the marker/antigen CD19 can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for CD19 can have a K_{d} value of greater than 10⁻⁷ to 10⁻¹⁰ M, as determined using an in vitro binding assay.

### Anti-HER-2 Binding Domains:

In some embodiments of the compositions of this disclosure, the binding domain can have specific binding affinity to the marker/antigen HER-2. Some embodiments of the compositions of this disclosure can comprise a bispecific bioactive assembly comprising the binding domain specific for HER-2 and another binding domain (e.g., having specific binding affinity to an effector cell). The binding domain can comprise VL and VH derived from a monoclonal antibody to HER-2. Monoclonal antibodies to HER-2 are known in the art. Exemplary, non-limiting example(s) of HER-2 monoclonal antibodies and the VL and VH sequences thereof are presented in Table 6. Some embodiments of the binding domain with binding affinity to the marker/antigen HER-2 can comprise anti-HER-2 VL and VH sequence(s) set forth in Table 6. Some embodiments of the binding domain with binding affinity to the marker/antigen HER-2 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-HER-2 antibody/antibodies of Table 6. Some embodiments of the binding domain with binding affinity to the marker/antigen HER-2 can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for HER-2 can have a K_{d} value of greater than 10⁻⁷ to 10⁻¹⁰ M, as determined using an in vitro binding assay.

### Anti-HER-3 Binding Domains:

In some embodiments of the compositions of this disclosure, the binding domain can have specific binding affinity to the marker/antigen HER-3. Some embodiments of the compositions of this disclosure can comprise a bispecific bioactive assembly comprising the binding domain specific for HER-3 and another binding domain (e.g., having specific binding affinity to an effector cell). The binding domain can comprise VL and VH derived from a monoclonal antibody to HER-3. Monoclonal antibodies to HER-3 are known in the art. Exemplary, non-limiting example(s) of HER-3 monoclonal antibodies and the VL and VH sequences thereof are presented in Table 6. Some embodiments of the binding domain with binding affinity to the marker/antigen HER-3 can comprise anti-HER-3 VL and VH sequence(s) set forth in Table 6. Some embodiments of the binding domain with binding affinity to the marker/antigen HER-3 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-HER-3 antibody/antibodies of Table 6. Some embodiments of the binding domain with binding affinity to the marker/antigen HER-3 can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for HER-3 can have a K_{d} value of greater than 10⁻⁷ to 10⁻¹⁰ M, as determined using an in vitro binding assay.

### Anti-HER-4 Binding Domains:

In some embodiments of the compositions of this disclosure, the binding domain can have specific binding affinity to the marker/antigen HER-4. Some embodiments of the compositions of this disclosure can comprise a bispecific bioactive assembly comprising the binding domain specific for HER-4 and another binding domain (e.g., having specific binding affinity to an effector cell). The binding domain can comprise VL and VH derived from a monoclonal antibody to HER-4. Monoclonal antibodies to HER-4 are known in the art. Exemplary, non-limiting example(s) of HER-4 monoclonal antibodies and the VL and VH sequences thereof are presented in Table 6. Some embodiments of the binding domain with binding affinity to the marker/antigen HER-4 can comprise anti-HER-4 VL and VH sequence(s) set forth in Table 6. Some embodiments of the binding domain with binding affinity to the marker/antigen HER-4 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-HER-4 antibody/antibodies of Table 6. Some embodiments of the binding domain with binding affinity to the marker/antigen HER-4 can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for HER-4 can have a K_{d} value of greater than 10⁻⁷ to 10⁻¹⁰ M, as determined using an in vitro binding assay.

### Anti-EGFR (Epidermal Growth Factor Receptor) Binding Domains:

In some embodiments of the compositions of this disclosure, the binding domain can have specific binding affinity to the marker/antigen EGFR. Some embodiments of the compositions of this disclosure can comprise a bispecific bioactive assembly comprising the binding domain specific for EGFR and another binding domain (e.g., having specific binding affinity to an effector cell). The binding domain can comprise VL and VH derived from a monoclonal antibody to EGFR. Monoclonal antibodies to EGFR are known in the art. Exemplary, non-limiting example(s) of EGFR monoclonal antibodies and the VL and VH sequences thereof are presented in Table 6. Some embodiments of the binding domain with binding affinity to the marker/antigen EGFR can comprise anti-EGFR VL and VH sequence(s) set forth in Table 6. Some embodiments of the binding domain with binding affinity to the marker/antigen EGFR can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-EGFR antibody/antibodies of Table 6. Some embodiments of the binding domain with binding affinity to the marker/antigen EGFR can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for EGFR can have a K_{d} value of greater than 10⁻⁷ to 10⁻¹⁰ M, as determined using an in vitro binding assay.

### Anti-PSMA Binding Domains:

In some embodiments of the compositions of this disclosure, the binding domain can have specific binding affinity to the marker/antigen PSMA (prostate-specific membrane antigen). Some embodiments of the compositions of this disclosure can comprise a bispecific bioactive assembly comprising the binding domain specific for PSMA and another binding domain (e.g., having specific binding affinity to an effector cell). The binding domain can comprise VL and VH derived from a monoclonal antibody to PSMA. Monoclonal antibodies to PSMA are known in the art. Exemplary, non-limiting example(s) of PSMA monoclonal antibodies and the VL and VH sequences thereof are presented in Table 6. Some embodiments of the binding domain with binding affinity to the marker/antigen PSMA can comprise anti-PSMA VL and VH sequence(s) set forth in Table 6. Some embodiments of the binding domain with binding affinity to the marker/antigen PSMA can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-PSMA antibody/antibodies of Table 6. Some embodiments of the binding domain with binding affinity to the marker/antigen PSMA can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for PSMA can have a K_{d} value of greater than 10⁻⁷ to 10⁻¹⁰ M, as determined using an in vitro binding assay.

### Anti-CEA Binding Domains:

In some embodiments of the compositions of this disclosure, the binding domain can have specific binding affinity to the marker/antigen CEA (carcinoembryonic antigen). Some embodiments of the compositions of this disclosure can comprise a bispecific bioactive assembly comprising the binding domain specific for CEA and another binding domain (e.g., having specific binding affinity to an effector cell). The binding domain can comprise VL and VH derived from a monoclonal antibody to CEA. Monoclonal antibodies to CEA are known in the art. Exemplary, non-limiting example(s) of CEA monoclonal antibodies and the VL and VH sequences thereof are presented in Table 6. Some embodiments of the binding domain with binding affinity to the marker/antigen CEA can comprise anti-CEA VL and VH sequence(s) set forth in Table 6. Some embodiments of the binding domain with binding affinity to the marker/antigen CEA can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-CEA antibody/antibodies of Table 6. Some embodiments of the binding domain with binding affinity to the marker/antigen CEA can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for CEA can have a K_{d} value of greater than 10⁻⁷ to 10⁻¹⁰ M, as determined using an in vitro binding assay.

### Anti-MUC1 Binding Domains:

In some embodiments of the compositions of this disclosure, the binding domain can have specific binding affinity to the marker/antigen MUC1. Some embodiments of the compositions of this disclosure can comprise a bispecific bioactive assembly comprising the binding domain specific for MUC1 and another binding domain (e.g., having specific binding affinity to an effector cell). The binding domain can comprise VL and VH derived from a monoclonal antibody to MUC1. Monoclonal antibodies to MUC1 are known in the art. Exemplary, non-limiting example(s) of MUC1 monoclonal antibodies and the VL and VH sequences thereof are presented in Table 6. Some embodiments of the binding domain with binding affinity to the marker/antigen MUC1 can comprise anti-MUC1 VL and VH sequence(s) set forth in Table 6. Some embodiments of the binding domain with binding affinity to the marker/antigen MUC1 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-MUC1 antibody/antibodies of Table 6. Some embodiments of the binding domain with binding affinity to the marker/antigen MUC1 can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for MUC1 can have a K_{d} value of greater than 10⁻⁷ to 10⁻¹⁰ M, as determined using an in vitro binding assay.

### Anti-MUC2 Binding Domains:

In some embodiments of the compositions of this disclosure, the binding domain can have specific binding affinity to the marker/antigen MUC2. Some embodiments of the compositions of this disclosure can comprise a bispecific bioactive assembly comprising the binding domain specific for MUC2 and another binding domain (e.g., having specific binding affinity to an effector cell). The binding domain can comprise VL and VH derived from a monoclonal antibody to MUC2. Monoclonal antibodies to MUC2 are known in the art. Some embodiments of the binding domain with binding affinity to the marker/antigen MUC2 can comprise anti-MUC2 VL and VH sequence(s). Some embodiments of the binding domain with binding affinity to the marker/antigen MUC2 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-MUC2 antibody/antibodies. Some embodiments of the binding domain with binding affinity to the marker/antigen MUC2 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for MUC2 can have a K_{d} value of greater than 10⁻⁷ to 10⁻¹⁰ M, as determined using an in vitro binding assay.

### Anti-MUC3 Binding Domains:

In some embodiments of the compositions of this disclosure, the binding domain can have specific binding affinity to the marker/antigen MUC3. Some embodiments of the compositions of this disclosure can comprise a bispecific bioactive assembly comprising the binding domain specific for MUC3 and another binding domain (e.g., having specific binding affinity to an effector cell). The binding domain can comprise VL and VH derived from a monoclonal antibody to MUC3. Monoclonal antibodies to MUC3 are known in the art. Some embodiments of the binding domain with binding affinity to the marker/antigen MUC3 can comprise anti-MUC3 VL and VH sequence(s). Some embodiments of the binding domain with binding affinity to the marker/antigen MUC3 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-MUC3 antibody/antibodies. Some embodiments of the binding domain with binding affinity to the marker/antigen MUC3 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for MUC3 can have a K_{d} value of greater than 10⁻⁷ to 10⁻¹⁰ M, as determined using an in vitro binding assay.

### Anti-MUC4 Binding Domains:

In some embodiments of the compositions of this disclosure, the binding domain can have specific binding affinity to the marker/antigen MUC4. Some embodiments of the compositions of this disclosure can comprise a bispecific bioactive assembly comprising the binding domain specific for MUC4 and another binding domain (e.g., having specific binding affinity to an effector cell). The binding domain can comprise VL and VH derived from a monoclonal antibody to MUC4. Monoclonal antibodies to MUC4 are known in the art. Some embodiments of the binding domain with binding affinity to the marker/antigen MUC4 can comprise anti-MUC4 VL and VH sequence(s). Some embodiments of the binding domain with binding affinity to the marker/antigen MUC4 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-MUC4 antibody/antibodies. Some embodiments of the binding domain with binding affinity to the marker/antigen MUC4 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for MUC4 can have a K_{d} value of greater than 10⁻⁷ to 10⁻¹⁰ M, as determined using an in vitro binding assay.

### Anti-MUC5AC Binding Domains:

In some embodiments of the compositions of this disclosure, the binding domain can have specific binding affinity to the marker/antigen MUC5AC. Some embodiments of the compositions of this disclosure can comprise a bispecific bioactive assembly comprising the binding domain specific for MUC5AC and another binding domain (e.g., having specific binding affinity to an effector cell). The binding domain can comprise VL and VH derived from a monoclonal antibody to MUC5AC. Monoclonal antibodies to MUC5AC are known in the art. Some embodiments of the binding domain with binding affinity to the marker/antigen MUC5AC can comprise anti-MUC5AC VL and VH sequence(s). Some embodiments of the binding domain with binding affinity to the marker/antigen MUC5AC can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-MUC5AC antibody/antibodies. Some embodiments of the binding domain with binding affinity to the marker/antigen MUC5AC can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for MUC5AC can have a K_{d} value of greater than 10⁻⁷ to 10⁻¹⁰ M, as determined using an in vitro binding assay.

### Anti-MUC5B Binding Domains:

In some embodiments of the compositions of this disclosure, the binding domain can have specific binding affinity to the marker/antigen MUC5B. Some embodiments of the compositions of this disclosure can comprise a bispecific bioactive assembly comprising the binding domain specific for MUC5B and another binding domain (e.g., having specific binding affinity to an effector cell). The binding domain can comprise VL and VH derived from a monoclonal antibody to MUC5B. Monoclonal antibodies to MUC5B are known in the art. Some embodiments of the binding domain with binding affinity to the marker/antigen MUC5B can comprise anti-MUC5B VL and VH sequence(s). Some embodiments of the binding domain with binding affinity to the marker/antigen MUC5B can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-MUC5B antibody/antibodies. Some embodiments of the binding domain with binding affinity to the marker/antigen MUC5B can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for MUC5B can have a K_{d} value of greater than 10⁻⁷ to 10⁻¹⁰ M, as determined using an in vitro binding assay.

### Anti-MUC7 Binding Domains:

In some embodiments of the compositions of this disclosure, the binding domain can have specific binding affinity to the marker/antigen MUC7. Some embodiments of the compositions of this disclosure can comprise a bispecific bioactive assembly comprising the binding domain specific for MUC7 and another binding domain (e.g., having specific binding affinity to an effector cell). The binding domain can comprise VL and VH derived from a monoclonal antibody to MUC7. Monoclonal antibodies to MUC7 are known in the art. Some embodiments of the binding domain with binding affinity to the marker/antigen MUC7 can comprise anti-MUC7 VL and VH sequence(s). Some embodiments of the binding domain with binding affinity to the marker/antigen MUC7 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-MUC7 antibody/antibodies. Some embodiments of the binding domain with binding affinity to the marker/antigen MUC7 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for MUC7 can have a K_{d} value of greater than 10⁻⁷ to 10⁻¹⁰ M, as determined using an in vitro binding assay.

### Anti-bhCG Binding Domains:

In some embodiments of the compositions of this disclosure, the binding domain can have specific binding affinity to the marker/antigen βhCG. Some embodiments of the compositions of this disclosure can comprise a bispecific bioactive assembly comprising the binding domain specific for βhCG and another binding domain (e.g., having specific binding affinity to an effector cell). The binding domain can comprise VL and VH derived from a monoclonal antibody to βhCG. Monoclonal antibodies to βhCG are known in the art. Some embodiments of the binding domain with binding affinity to the marker/antigen βhCG can comprise anti-βhCG VL and VH sequence(s). Some embodiments of the binding domain with binding affinity to the marker/antigen βhCG can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-βhCG antibody/antibodies. Some embodiments of the binding domain with binding affinity to the marker/antigen βhCG can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for βhCG can have a K_{d} value of greater than 10⁻⁷ to 10⁻¹⁰ M, as determined using an in vitro binding assay.

### Anti-Lewis-Y Binding Domains:

In some embodiments of the compositions of this disclosure, the binding domain can have specific binding affinity to the marker/antigen Lewis-Y. Some embodiments of the compositions of this disclosure can comprise a bispecific bioactive assembly comprising the binding domain specific for Lewis-Y and another binding domain (e.g., having specific binding affinity to an effector cell). The binding domain can comprise VL and VH derived from a monoclonal antibody to Lewis-Y. Monoclonal antibodies to Lewis-Y are known in the art. Exemplary, non-limiting example(s) of Lewis-Y monoclonal antibodies and the VL and VH sequences thereof are presented in Table 6. Some embodiments of the binding domain with binding affinity to the marker/antigen Lewis-Y can comprise anti-Lewis-Y VL and VH sequence(s) set forth in Table 6. Some embodiments of the binding domain with binding affinity to the marker/antigen Lewis-Y can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-Lewis-Y antibody/antibodies of Table 6. Some embodiments of the binding domain with binding affinity to the marker/antigen Lewis-Y can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for Lewis-Y can have a K_{d} value of greater than 10⁻⁷ to 10⁻¹⁰ M, as determined using an in vitro binding assay.

### Anti-CD20 Binding Domains

In some embodiments of the compositions of this disclosure, the binding domain can have specific binding affinity to the marker/antigen CD20. Some embodiments of the compositions of this disclosure can comprise a bispecific bioactive assembly comprising the binding domain specific for CD20 and another binding domain (e.g., having specific binding affinity to an effector cell). The binding domain can comprise VL and VH derived from a monoclonal antibody to CD20. Monoclonal antibodies to CD20 are known in the art. Exemplary, non-limiting example(s) of CD20 monoclonal antibodies and the VL and VH sequences thereof are presented in Table 6. Some embodiments of the binding domain with binding affinity to the marker/antigen CD20 can comprise anti-CD20 VL and VH sequence(s) set forth in Table 6. Some embodiments of the binding domain with binding affinity to the marker/antigen CD20 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-CD20 antibody/antibodies of Table 6. Some embodiments of the binding domain with binding affinity to the marker/antigen CD20 can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for CD20 can have a K_{d} value of greater than 10⁻⁷ to 10⁻¹⁰ M, as determined using an in vitro binding assay.

### Anti-CD33 Binding Domains

In some embodiments of the compositions of this disclosure, the binding domain can have specific binding affinity to the marker/antigen CD33. Some embodiments of the compositions of this disclosure can comprise a bispecific bioactive assembly comprising the binding domain specific for CD33 and another binding domain (e.g., having specific binding affinity to an effector cell). The binding domain can comprise VL and VH derived from a monoclonal antibody to CD33. Monoclonal antibodies to CD33 are known in the art. Exemplary, non-limiting example(s) of CD33 monoclonal antibodies and the VL and VH sequences thereof are presented in Table 6. Some embodiments of the binding domain with binding affinity to the marker/antigen CD33 can comprise anti-CD33 VL and VH sequence(s) set forth in Table 6. Some embodiments of the binding domain with binding affinity to the marker/antigen CD33 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-CD33 antibody/antibodies of Table 6. Some embodiments of the binding domain with binding affinity to the marker/antigen CD33 can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for CD33 can have a K_{d} value of greater than 10⁻⁷ to 10⁻¹⁰ M, as determined using an in vitro binding assay.

### Anti-CD30 Binding Domains:

In some embodiments of the compositions of this disclosure, the binding domain can have specific binding affinity to the marker/antigen CD30. Some embodiments of the compositions of this disclosure can comprise a bispecific bioactive assembly comprising the binding domain specific for CD30 and another binding domain (e.g., having specific binding affinity to an effector cell). The binding domain can comprise VL and VH derived from a monoclonal antibody to CD30. Monoclonal antibodies to CD30 are known in the art. Some embodiments of the binding domain with binding affinity to the marker/antigen CD30 can comprise anti-CD30 VL and VH sequence(s). Some embodiments of the binding domain with binding affinity to the marker/antigen CD30 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-CD30 antibody/antibodies. Some embodiments of the binding domain with binding affinity to the marker/antigen CD30 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for CD30 can have a K_{d} value of greater than 10⁻⁷ to 10⁻¹⁰ M, as determined using an in vitro binding assay.

### Anti-Ganglioside GD3 Binding Domains:

In some embodiments of the compositions of this disclosure, the binding domain can have specific binding affinity to the marker/antigen ganglioside GD3. Some embodiments of the compositions of this disclosure can comprise a bispecific bioactive assembly comprising the binding domain specific for ganglioside GD3 and another binding domain (e.g., having specific binding affinity to an effector cell). The binding domain can comprise VL and VH derived from a monoclonal antibody to ganglioside GD3. Monoclonal antibodies to ganglioside GD3 are known in the art. Some embodiments of the binding domain with binding affinity to the marker/antigen ganglioside GD3 can comprise anti-ganglioside GD3 VL and VH sequence(s). Some embodiments of the binding domain with binding affinity to the marker/antigen ganglioside GD3 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-ganglioside GD3 antibody/antibodies. Some embodiments of the binding domain with binding affinity to the marker/antigen ganglioside GD3 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for ganglioside GD3 can have a K_{d} value of greater than 10⁻⁷ to 10⁻¹⁰ M, as determined using an in vitro binding assay.

### Anti-9-O-Acetyl-GD3 Binding Domains:

In some embodiments of the compositions of this disclosure, the binding domain can have specific binding affinity to the marker/antigen 9-O-Acetyl-GD3. Some embodiments of the compositions of this disclosure can comprise a bispecific bioactive assembly comprising the binding domain specific for 9-O-Acetyl-GD3 and another binding domain (e.g., having specific binding affinity to an effector cell). The binding domain can comprise VL and VH derived from a monoclonal antibody to 9-O-Acetyl-GD3. Monoclonal antibodies to 9-O-Acetyl-GD3 are known in the art. Some embodiments of the binding domain with binding affinity to the marker/antigen 9-O-Acetyl-GD3 can comprise anti-9-O-Acetyl-GD3 VL and VH sequence(s). Some embodiments of the binding domain with binding affinity to the marker/antigen 9-O-Acetyl-GD3 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-9-O-Acetyl-GD3 antibody/antibodies. Some embodiments of the binding domain with binding affinity to the marker/antigen 9-O-Acetyl-GD3 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for 9-O-Acetyl-GD3 can have a K_{d} value of greater than 10⁻⁷ to 10⁻¹⁰ M, as determined using an in vitro binding assay.

### Anti-Globo H Binding Domains:

In some embodiments of the compositions of this disclosure, the binding domain can have specific binding affinity to the marker/antigen globo H. Some embodiments of the compositions of this disclosure can comprise a bispecific bioactive assembly comprising the binding domain specific for globo H and another binding domain (e.g., having specific binding affinity to an effector cell). The binding domain can comprise VL and VH derived from a monoclonal antibody to globo H. Monoclonal antibodies to globo H are known in the art. Some embodiments of the binding domain with binding affinity to the marker/antigen globo H can comprise anti-globo H VL and VH sequence(s). Some embodiments of the binding domain with binding affinity to the marker/antigen globo H can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-globo H antibody/antibodies. Some embodiments of the binding domain with binding affinity to the marker/antigen globo H can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for globo H can have a K_{d} value of greater than 10⁻⁷ to 10⁻¹⁰ M, as determined using an in vitro binding assay.

### Anti-Fucosyl GM1 Binding Domains:

In some embodiments of the compositions of this disclosure, the binding domain can have specific binding affinity to the marker/antigen fucosyl GM1. Some embodiments of the compositions of this disclosure can comprise a bispecific bioactive assembly comprising the binding domain specific for fucosyl GM1 and another binding domain (e.g., having specific binding affinity to an effector cell). The binding domain can comprise VL and VH derived from a monoclonal antibody to fucosyl GM1. Monoclonal antibodies to fucosyl GM1 are known in the art. Some embodiments of the binding domain with binding affinity to the marker/antigen fucosyl GM1 can comprise anti-fucosyl GM1 VL and VH sequence(s). Some embodiments of the binding domain with binding affinity to the marker/antigen fucosyl GM1 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-fucosyl GM1 antibody/antibodies. Some embodiments of the binding domain with binding affinity to the marker/antigen fucosyl GM1 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for fucosyl GM1 can have a K_{d} value of greater than 10⁻⁷ to 10⁻¹⁰ M, as determined using an in vitro binding assay.

### Anti-GD2 Binding Domains:

In some embodiments of the compositions of this disclosure, the binding domain can have specific binding affinity to the marker/antigen GD2. Some embodiments of the compositions of this disclosure can comprise a bispecific bioactive assembly comprising the binding domain specific for GD2 and another binding domain (e.g., having specific binding affinity to an effector cell). The binding domain can comprise VL and VH derived from a monoclonal antibody to GD2. Monoclonal antibodies to GD2 are known in the art. Some embodiments of the binding domain with binding affinity to the marker/antigen GD2 can comprise anti-GD2 VL and VH sequence(s). Some embodiments of the binding domain with binding affinity to the marker/antigen GD2 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-GD2 antibody/antibodies. Some embodiments of the binding domain with binding affinity to the marker/antigen GD2 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for GD2 can have a K_{d} value of greater than 10⁻⁷ to 10⁻¹⁰ M, as determined using an in vitro binding assay.

### Anti-Carbonicanhydrase IX Binding Domains:

In some embodiments of the compositions of this disclosure, the binding domain can have specific binding affinity to the marker/antigen CA IX (carbonicanhydrase IX). Some embodiments of the compositions of this disclosure can comprise a bispecific bioactive assembly comprising the binding domain specific for CA IX and another binding domain (e.g., having specific binding affinity to an effector cell). The binding domain can comprise VL and VH derived from a monoclonal antibody to CA IX. Monoclonal antibodies to CA IX are known in the art. Some embodiments of the binding domain with binding affinity to the marker/antigen CA IX can comprise anti-CA IX VL and VH sequence(s). Some embodiments of the binding domain with binding affinity to the marker/antigen CA IX can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-CA IX antibody/antibodies. Some embodiments of the binding domain with binding affinity to the marker/antigen CA IX can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for CA IX can have a K_{d} value of greater than 10⁻⁷ to 10⁻¹⁰ M, as determined using an in vitro binding assay.

### Anti-CD44v6 Binding Domains:

In some embodiments of the compositions of this disclosure, the binding domain can have specific binding affinity to the marker/antigen CD44v6. Some embodiments of the compositions of this disclosure can comprise a bispecific bioactive assembly comprising the binding domain specific for CD44v6 and another binding domain (e.g., having specific binding affinity to an effector cell). The binding domain can comprise VL and VH derived from a monoclonal antibody to CD44v6. Monoclonal antibodies to CD44v6 are known in the art. Some embodiments of the binding domain with binding affinity to the marker/antigen CD44v6 can comprise anti-CD44v6 VL and VH sequence(s). Some embodiments of the binding domain with binding affinity to the marker/antigen CD44v6 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-CD44v6 antibody/antibodies. Some embodiments of the binding domain with binding affinity to the marker/antigen CD44v6 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for CD44v6 can have a K_{d} value of greater than 10⁻⁷ to 10⁻¹⁰ M, as determined using an in vitro binding assay.

### Anti-Sonic Hedgehog (Shh) Binding Domains:

In some embodiments of the compositions of this disclosure, the binding domain can have specific binding affinity to the marker/antigen Shh (sonic hedgehog). Some embodiments of the compositions of this disclosure can comprise a bispecific bioactive assembly comprising the binding domain specific for Shh and another binding domain (e.g., having specific binding affinity to an effector cell). The binding domain can comprise VL and VH derived from a monoclonal antibody to Shh. Monoclonal antibodies to Shh are known in the art. Some embodiments of the binding domain with binding affinity to the marker/antigen Shh can comprise anti-Shh VL and VH sequence(s). Some embodiments of the binding domain with binding affinity to the marker/antigen Shh can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-Shh antibody/antibodies. Some embodiments of the binding domain with binding affinity to the marker/antigen Shh can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for Shh can have a K_{d} value of greater than 10⁻⁷ to 10⁻¹⁰ M, as determined using an in vitro binding assay.

### Anti-Wue-1 Binding Domains:

In some embodiments of the compositions of this disclosure, the binding domain can have specific binding affinity to the marker/antigen Wue-1. Some embodiments of the compositions of this disclosure can comprise a bispecific bioactive assembly comprising the binding domain specific for Wue-1 and another binding domain (e.g., having specific binding affinity to an effector cell). The binding domain can comprise VL and VH derived from a monoclonal antibody to Wue-1. Monoclonal antibodies to Wue-1 are known in the art. Some embodiments of the binding domain with binding affinity to the marker/antigen Wue-1 can comprise anti-Wue-1 VL and VH sequence(s). Some embodiments of the binding domain with binding affinity to the marker/antigen Wue-1 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-Wue-1 antibody/antibodies. Some embodiments of the binding domain with binding affinity to the marker/antigen Wue-1 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for Wue-1 can have a K_{d} value of greater than 10⁻⁷ to 10⁻¹⁰ M, as determined using an in vitro binding assay.

### Anti-Plasma Cell Antigen 1 (PC-1) Binding Domains:

In some embodiments of the compositions of this disclosure, the binding domain can have specific binding affinity to the marker/antigen PC-1 (plasma cell antigen). Some embodiments of the compositions of this disclosure can comprise a bispecific bioactive assembly comprising the binding domain specific for PC-1 (plasma cell antigen) and another binding domain (e.g., having specific binding affinity to an effector cell). The binding domain can comprise VL and VH derived from a monoclonal antibody to PC-1 (plasma cell antigen). Monoclonal antibodies to PC-1 (plasma cell antigen) are known in the art. Some embodiments of the binding domain with binding affinity to the marker/antigen PC-1 (plasma cell antigen) can comprise anti-PC-1 (plasma cell antigen) VL and VH sequence(s). Some embodiments of the binding domain with binding affinity to the marker/antigen PC-1 (plasma cell antigen) can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-PC-1 (plasma cell antigen) antibody/antibodies. Some embodiments of the binding domain with binding affinity to the marker/antigen PC-1 (plasma cell antigen) can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for PC-1 (plasma cell antigen) can have a K_{d} value of greater than 10⁻⁷ to 10⁻¹⁰ M, as determined using an in vitro binding assay.

### Anti-Melanoma Chondroitin Sulfate Proteoglycan (MCSP) Binding Domains:

In some embodiments of the compositions of this disclosure, the binding domain can have specific binding affinity to the marker/antigen MCSP (melanoma chondroitin sulfate proteoglycan). Some embodiments of the compositions of this disclosure can comprise a bispecific bioactive assembly comprising the binding domain specific for MCSP and another binding domain (e.g., having specific binding affinity to an effector cell). The binding domain can comprise VL and VH derived from a monoclonal antibody to MCSP. Monoclonal antibodies to MCSP are known in the art. Some embodiments of the binding domain with binding affinity to the marker/antigen MCSP can comprise anti-MCSP VL and VH sequence(s). Some embodiments of the binding domain with binding affinity to the marker/antigen MCSP can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-MCSP antibody/antibodies. Some embodiments of the binding domain with binding affinity to the marker/antigen MCSP can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for MCSP can have a K_{d} value of greater than 10⁻⁷ to 10⁻¹⁰ M, as determined using an in vitro binding assay.

### Anti-CCR8 Binding Domains:

In some embodiments of the compositions of this disclosure, the binding domain can have specific binding affinity to the marker/antigen CCR8. Some embodiments of the compositions of this disclosure can comprise a bispecific bioactive assembly comprising the binding domain specific for CCR8 and another binding domain (e.g., having specific binding affinity to an effector cell). The binding domain can comprise VL and VH derived from a monoclonal antibody to CCR8. Monoclonal antibodies to CCR8 are known in the art. Some embodiments of the binding domain with binding affinity to the marker/antigen CCR8 can comprise anti-CCR8 VL and VH sequence(s). Some embodiments of the binding domain with binding affinity to the marker/antigen CCR8 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-CCR8 antibody/antibodies. Some embodiments of the binding domain with binding affinity to the marker/antigen CCR8 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for CCR8 can have a K_{d} value of greater than 10⁻⁷ to 10⁻¹⁰ M, as determined using an in vitro binding assay.

### Anti-6-Transmembrane Epithelial Antigen of Prostate (STEAP) Binding Domains:

In some embodiments of the compositions of this disclosure, the binding domain can have specific binding affinity to the marker/antigen STEAP. Some embodiments of the compositions of this disclosure can comprise a bispecific bioactive assembly comprising the binding domain specific for STEAP and another binding domain (e.g., having specific binding affinity to an effector cell). The binding domain can comprise VL and VH derived from a monoclonal antibody to STEAP. Monoclonal antibodies to STEAP are known in the art. Some embodiments of the binding domain with binding affinity to the marker/antigen STEAP can comprise anti-STEAP VL and VH sequence(s). Some embodiments of the binding domain with binding affinity to the marker/antigen STEAP can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-STEAP antibody/antibodies. Some embodiments of the binding domain with binding affinity to the marker/antigen STEAP can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for STEAP can have a K_{d} value of greater than 10⁻⁷ to 10⁻¹⁰ M, as determined using an in vitro binding assay.

### Anti-Mesothelin Binding Domains:

In some embodiments of the compositions of this disclosure, the binding domain can have specific binding affinity to the marker/antigen mesothelin. Some embodiments of the compositions of this disclosure can comprise a bispecific bioactive assembly comprising the binding domain specific for mesothelin and another binding domain (e.g., having specific binding affinity to an effector cell). The binding domain can comprise VL and VH derived from a monoclonal antibody to mesothelin. Monoclonal antibodies to mesothelin are known in the art. Exemplary, non-limiting example(s) of mesothelin monoclonal antibodies and the VL and VH sequences thereof are presented in Table 6. Some embodiments of the binding domain with binding affinity to the marker/antigen mesothelin can comprise anti-mesothelin VL and VH sequence(s) set forth in Table 6. Some embodiments of the binding domain with binding affinity to the marker/antigen mesothelin can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-mesothelin antibody/antibodies of Table 6. Some embodiments of the binding domain with binding affinity to the marker/antigen mesothelin can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for mesothelin can have a K_{d} value of greater than 10⁻⁷ to 10⁻¹⁰ M, as determined using an in vitro binding assay.

### Anti-A33 Antigen Binding Domains:

In some embodiments of the compositions of this disclosure, the binding domain can have specific binding affinity to the marker/antigen A33. Some embodiments of the compositions of this disclosure can comprise a bispecific bioactive assembly comprising the binding domain specific for A33 and another binding domain (e.g., having specific binding affinity to an effector cell). The binding domain can comprise VL and VH derived from a monoclonal antibody to A33. Monoclonal antibodies to A33 are known in the art. Some embodiments of the binding domain with binding affinity to the marker/antigen A33 can comprise anti-A33 VL and VH sequence(s). Some embodiments of the binding domain with binding affinity to the marker/antigen A33 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-A33 antibody/antibodies. Some embodiments of the binding domain with binding affinity to the marker/antigen A33 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for A33 can have a K_{d} value of greater than 10⁻⁷ to 10⁻¹⁰ M, as determined using an in vitro binding assay.

### Anti-PSCA Binding Domains:

In some embodiments of the compositions of this disclosure, the binding domain can have specific binding affinity to the marker/antigen PSCA. Some embodiments of the compositions of this disclosure can comprise a bispecific bioactive assembly comprising the binding domain specific for PSCA and another binding domain (e.g., having specific binding affinity to an effector cell). The binding domain can comprise VL and VH derived from a monoclonal antibody to PSCA. Monoclonal antibodies to PSCA are known in the art. Some embodiments of the binding domain with binding affinity to the marker/antigen PSCA can comprise anti-PSCA VL and VH sequence(s). Some embodiments of the binding domain with binding affinity to the marker/antigen PSCA can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-PSCA antibody/antibodies. Some embodiments of the binding domain with binding affinity to the marker/antigen PSCA can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for PSCA can have a K_{d} value of greater than 10⁻⁷ to 10⁻¹⁰ M, as determined using an in vitro binding assay.

### Anti-Ly-6 Binding Domains:

In some embodiments of the compositions of this disclosure, the binding domain can have specific binding affinity to the marker/antigen Ly-6. Some embodiments of the compositions of this disclosure can comprise a bispecific bioactive assembly comprising the binding domain specific for Ly-6 and another binding domain (e.g., having specific binding affinity to an effector cell). The binding domain can comprise VL and VH derived from a monoclonal antibody to Ly-6. Monoclonal antibodies to Ly-6 are known in the art. Some embodiments of the binding domain with binding affinity to the marker/antigen Ly-6 can comprise anti-Ly-6 VL and VH sequence(s). Some embodiments of the binding domain with binding affinity to the marker/antigen Ly-6 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-Ly-6 antibody/antibodies. Some embodiments of the binding domain with binding affinity to the marker/antigen Ly-6 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for Ly-6 can have a K_{d} value of greater than 10⁻⁷ to 10⁻¹⁰ M, as determined using an in vitro binding assay.

### Anti-SAS Binding Domains:

In some embodiments of the compositions of this disclosure, the binding domain can have specific binding affinity to the marker/antigen SAS. Some embodiments of the compositions of this disclosure can comprise a bispecific bioactive assembly comprising the binding domain specific for SAS and another binding domain (e.g., having specific binding affinity to an effector cell). The binding domain can comprise VL and VH derived from a monoclonal antibody to SAS. Monoclonal antibodies to SAS are known in the art. Some embodiments of the binding domain with binding affinity to the marker/antigen SAS can comprise anti-SAS VL and VH sequence(s). Some embodiments of the binding domain with binding affinity to the marker/antigen SAS can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-SAS antibody/antibodies. Some embodiments of the binding domain with binding affinity to the marker/antigen SAS can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for SAS can have a K_{d} value of greater than 10⁻⁷ to 10⁻¹⁰ M, as determined using an in vitro binding assay.

### Anti-Desmoglein 4 Binding Domains:

In some embodiments of the compositions of this disclosure, the binding domain can have specific binding affinity to the marker/antigen desmoglein 4. Some embodiments of the compositions of this disclosure can comprise a bispecific bioactive assembly comprising the binding domain specific for desmoglein 4 and another binding domain (e.g., having specific binding affinity to an effector cell). The binding domain can comprise VL and VH derived from a monoclonal antibody to desmoglein 4. Monoclonal antibodies to desmoglein 4 are known in the art. Some embodiments of the binding domain with binding affinity to the marker/antigen desmoglein 4 can comprise anti-desmoglein 4 VL and VH sequence(s). Some embodiments of the binding domain with binding affinity to the marker/antigen desmoglein 4 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-desmoglein 4 antibody/antibodies. Some embodiments of the binding domain with binding affinity to the marker/antigen desmoglein 4 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for desmoglein 4 can have a K_{d} value of greater than 10⁻⁷ to 10⁻¹⁰ M, as determined using an in vitro binding assay.

### Anti-fnAChR (Fetal Acetylcholine Receptor) Binding Domains:

In some embodiments of the compositions of this disclosure, the binding domain can have specific binding affinity to the marker/antigen fnAChR (fetal acetylcholine receptor). Some embodiments of the compositions of this disclosure can comprise a bispecific bioactive assembly comprising the binding domain specific for fnAChR and another binding domain (e.g., having specific binding affinity to an effector cell). The binding domain can comprise VL and VH derived from a monoclonal antibody to fnAChR. Monoclonal antibodies to fnAChR are known in the art. Some embodiments of the binding domain with binding affinity to the marker/antigen fnAChR can comprise anti-fnAChR VL and VH sequence(s). Some embodiments of the binding domain with binding affinity to the marker/antigen fnAChR can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-fnAChR antibody/antibodies. Some embodiments of the binding domain with binding affinity to the marker/antigen fnAChR can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for fnAChR can have a K_{d} value of greater than 10⁻⁷ to 10⁻¹⁰ M, as determined using an in vitro binding assay.

### Anti-CD25 Binding Domains:

In some embodiments of the compositions of this disclosure, the binding domain can have specific binding affinity to the marker/antigen CD25. Some embodiments of the compositions of this disclosure can comprise a bispecific bioactive assembly comprising the binding domain specific for CD25 and another binding domain (e.g., having specific binding affinity to an effector cell). The binding domain can comprise VL and VH derived from a monoclonal antibody to CD25. Monoclonal antibodies to CD25 are known in the art. Some embodiments of the binding domain with binding affinity to the marker/antigen CD25 can comprise anti-CD25 VL and VH sequence(s). Some embodiments of the binding domain with binding affinity to the marker/antigen CD25 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-CD25 antibody/antibodies. Some embodiments of the binding domain with binding affinity to the marker/antigen CD25 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for CD25 can have a K_{d} value of greater than 10⁻⁷ to 10⁻¹⁰ M, as determined using an in vitro binding assay.

### Anti-Cancer Antigen 19-9 Binding Domains:

In some embodiments of the compositions of this disclosure, the binding domain can have specific binding affinity to the marker/antigen cancer antigen 19-9. Some embodiments of the compositions of this disclosure can comprise a bispecific bioactive assembly comprising the binding domain specific for cancer antigen 19-9 and another binding domain (e.g., having specific binding affinity to an effector cell). The binding domain can comprise VL and VH derived from a monoclonal antibody to cancer antigen 19-9. Monoclonal antibodies to cancer antigen 19-9 are known in the art. Some embodiments of the binding domain with binding affinity to the marker/antigen cancer antigen 19-9 can comprise anti-cancer antigen 19-9 VL and VH sequence(s). Some embodiments of the binding domain with binding affinity to the marker/antigen cancer antigen 19-9 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-cancer antigen 19-9 antibody/antibodies. Some embodiments of the binding domain with binding affinity to the marker/antigen cancer antigen 19-9 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for cancer antigen 19-9 (CA 19-9) can have a K_{d} value of greater than 10⁻⁷ to 10⁻¹⁰ M, as determined using an in vitro binding assay.

### Anti-Misiir Binding Domains:

In some embodiments of the compositions of this disclosure, the binding domain can have specific binding affinity to the marker/antigen MISIIR (müllerian inhibiting substance type II receptor). Some embodiments of the compositions of this disclosure can comprise a bispecific bioactive assembly comprising the binding domain specific for MISIIR and another binding domain (e.g., having specific binding affinity to an effector cell). The binding domain can comprise VL and VH derived from a monoclonal antibody to MISIIR. Monoclonal antibodies to MISIIR are known in the art. Some embodiments of the binding domain with binding affinity to the marker/antigen MISIIR can comprise anti-MISIIR VL and VH sequence(s). Some embodiments of the binding domain with binding affinity to the marker/antigen MISIIR can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-MISIIR antibody/antibodies. Some embodiments of the binding domain with binding affinity to the marker/antigen MISIIR can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for MISIIR can have a K_{d} value of greater than 10⁻⁷ to 10⁻¹⁰ M, as determined using an in vitro binding assay.

### Anti-sTn (Sialylated Tn Antigen) Binding Domains:

In some embodiments of the compositions of this disclosure, the binding domain can have specific binding affinity to the marker/antigen sTn (sialylated to antigen). Some embodiments of the compositions of this disclosure can comprise a bispecific bioactive assembly comprising the binding domain specific for sTn and another binding domain (e.g., having specific binding affinity to an effector cell). The binding domain can comprise VL and VH derived from a monoclonal antibody to sTn. Monoclonal antibodies to sTn are known in the art. Some embodiments of the binding domain with binding affinity to the marker/antigen sTn can comprise anti-sTn VL and VH sequence(s). Some embodiments of the binding domain with binding affinity to the marker/antigen sTn can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-sTn antibody/antibodies. Some embodiments of the binding domain with binding affinity to the marker/antigen sTn can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for sTn can have a K_{d} value of greater than 10⁻⁷ to 10⁻¹⁰ M, as determined using an in vitro binding assay.

### Anti-FAP Binding Domains:

In some embodiments of the compositions of this disclosure, the binding domain can have specific binding affinity to the marker/antigen FAP. Some embodiments of the compositions of this disclosure can comprise a bispecific bioactive assembly comprising the binding domain specific for FAP and another binding domain (e.g., having specific binding affinity to an effector cell). The binding domain can comprise VL and VH derived from a monoclonal antibody to FAP. Monoclonal antibodies to FAP are known in the art. Some embodiments of the binding domain with binding affinity to the marker/antigen FAP can comprise anti-FAP VL and VH sequence(s). Some embodiments of the binding domain with binding affinity to the marker/antigen FAP can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-FAP antibody/antibodies. Some embodiments of the binding domain with binding affinity to the marker/antigen FAP can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for FAP can have a K_{d} value of greater than 10⁻⁷ to 10⁻¹⁰ M, as determined using an in vitro binding assay.

### Anti-CD248 Binding Domains:

In some embodiments of the compositions of this disclosure, the binding domain can have specific binding affinity to the marker/antigen CD248. Some embodiments of the compositions of this disclosure can comprise a bispecific bioactive assembly comprising the binding domain specific for CD248 and another binding domain (e.g., having specific binding affinity to an effector cell). The binding domain can comprise VL and VH derived from a monoclonal antibody to CD248. Monoclonal antibodies to CD248 are known in the art. Some embodiments of the binding domain with binding affinity to the marker/antigen CD248 can comprise anti-CD248 VL and VH sequence(s). Some embodiments of the binding domain with binding affinity to the marker/antigen CD248 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-CD248 antibody/antibodies. Some embodiments of the binding domain with binding affinity to the marker/antigen CD248 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for CD248 can have a K_{d} value of greater than 10⁻⁷ to 10⁻¹⁰ M, as determined using an in vitro binding assay.

### Anti-EGFRvIII Binding Domains:

In some embodiments of the compositions of this disclosure, the binding domain can have specific binding affinity to the marker/antigen EGFRvIII. Some embodiments of the compositions of this disclosure can comprise a bispecific bioactive assembly comprising the binding domain specific for EGFRvIII and another binding domain (e.g., having specific binding affinity to an effector cell). The binding domain can comprise VL and VH derived from a monoclonal antibody to EGFRvIII. Monoclonal antibodies to EGFRvIII are known in the art. Some embodiments of the binding domain with binding affinity to the marker/antigen EGFRvIII can comprise anti-EGFRvIII VL and VH sequence(s). Some embodiments of the binding domain with binding affinity to the marker/antigen EGFRvIII can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-EGFRvIII antibody/antibodies. Some embodiments of the binding domain with binding affinity to the marker/antigen EGFRvIII can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for EGFRvIII can have a K_{d} value of greater than 10⁻⁷ to 10⁻¹⁰ M, as determined using an in vitro binding assay.

### Anti-TAL6 Binding Domains:

In some embodiments of the compositions of this disclosure, the binding domain can have specific binding affinity to the marker/antigen TAL6. Some embodiments of the compositions of this disclosure can comprise a bispecific bioactive assembly comprising the binding domain specific for TAL6 and another binding domain (e.g., having specific binding affinity to an effector cell). The binding domain can comprise VL and VH derived from a monoclonal antibody to TAL6. Monoclonal antibodies to TAL6 are known in the art. Some embodiments of the binding domain with binding affinity to the marker/antigen TAL6 can comprise anti-TAL6 VL and VH sequence(s). Some embodiments of the binding domain with binding affinity to the marker/antigen TAL6 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-TAL6 antibody/antibodies. Some embodiments of the binding domain with binding affinity to the marker/antigen TAL6 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for TAL6 can have a K_{d} value of greater than 10⁻⁷ to 10⁻¹⁰ M, as determined using an in vitro binding assay.

### Anti-CD63 Binding Domains:

In some embodiments of the compositions of this disclosure, the binding domain can have specific binding affinity to the marker/antigen CD63. Some embodiments of the compositions of this disclosure can comprise a bispecific bioactive assembly comprising the binding domain specific for CD63 and another binding domain (e.g., having specific binding affinity to an effector cell). The binding domain can comprise VL and VH derived from a monoclonal antibody to CD63. Monoclonal antibodies to CD63 are known in the art. Some embodiments of the binding domain with binding affinity to the marker/antigen CD63 can comprise anti-CD63 VL and VH sequence(s). Some embodiments of the binding domain with binding affinity to the marker/antigen CD63 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-CD63 antibody/antibodies. Some embodiments of the binding domain with binding affinity to the marker/antigen CD63 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for CD63 can have a K_{d} value of greater than 10⁻⁷ to 10⁻¹⁰ M, as determined using an in vitro binding assay.

### Anti-TAG72 Binding Domains:

In some embodiments of the compositions of this disclosure, the binding domain can have specific binding affinity to the marker/antigen TAG72. Some embodiments of the compositions of this disclosure can comprise a bispecific bioactive assembly comprising the binding domain specific for TAG72 and another binding domain (e.g., having specific binding affinity to an effector cell). The binding domain can comprise VL and VH derived from a monoclonal antibody to TAG72. Monoclonal antibodies to TAG72 are known in the art. Exemplary, non-limiting example(s) of TAG72 monoclonal antibodies and the VL and VH sequences thereof are presented in Table 6. Some embodiments of the binding domain with binding affinity to the marker/antigen TAG72 can comprise anti-TAG72 VL and VH sequence(s) set forth in Table 6. Some embodiments of the binding domain with binding affinity to the marker/antigen TAG72 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-TAG72 antibody/antibodies of Table 6. Some embodiments of the binding domain with binding affinity to the marker/antigen TAG72 can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for TAG72 can have a K_{d} value of greater than 10⁻⁷ to 10⁻¹⁰ M, as determined using an in vitro binding assay.

### Anti-TF-Antigen Binding Domains:

In some embodiments of the compositions of this disclosure, the binding domain can have specific binding affinity to the marker/antigen TF antigen. Some embodiments of the compositions of this disclosure can comprise a bispecific bioactive assembly comprising the binding domain specific for TF antigen and another binding domain (e.g., having specific binding affinity to an effector cell). The binding domain can comprise VL and VH derived from a monoclonal antibody to TF antigen. Monoclonal antibodies to TF antigen are known in the art. Some embodiments of the binding domain with binding affinity to the marker/antigen TF antigen can comprise anti-TF antigen VL and VH sequence(s). Some embodiments of the binding domain with binding affinity to the marker/antigen TF antigen can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-TF antigen antibody/antibodies. Some embodiments of the binding domain with binding affinity to the marker/antigen TF antigen can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for TF antigen can have a K_{d} value of greater than 10⁻⁷ to 10⁻¹⁰ M, as determined using an in vitro binding assay.

### Anti-IGF-IR Binding Domains:

In some embodiments of the compositions of this disclosure, the binding domain can have specific binding affinity to the marker/antigen IGF-IR. Some embodiments of the compositions of this disclosure can comprise a bispecific bioactive assembly comprising the binding domain specific for IGF-IR and another binding domain (e.g., having specific binding affinity to an effector cell). The binding domain can comprise VL and VH derived from a monoclonal antibody to IGF-IR. Monoclonal antibodies to IGF-IR are known in the art. Some embodiments of the binding domain with binding affinity to the marker/antigen IGF-IR can comprise anti-IGF-IR VL and VH sequence(s). Some embodiments of the binding domain with binding affinity to the marker/antigen IGF-IR can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-IGF-IR antibody/antibodies. Some embodiments of the binding domain with binding affinity to the marker/antigen IGF-IR can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for IGF-IR can have a K_{d} value of greater than 10⁻⁷ to 10⁻¹⁰ M, as determined using an in vitro binding assay.

### Anti-Cora Antigen Binding Domains:

In some embodiments of the compositions of this disclosure, the binding domain can have specific binding affinity to the marker/antigen cora antigen. Some embodiments of the compositions of this disclosure can comprise a bispecific bioactive assembly comprising the binding domain specific for cora antigen and another binding domain (e.g., having specific binding affinity to an effector cell). The binding domain can comprise VL and VH derived from a monoclonal antibody to cora antigen. Monoclonal antibodies to cora antigen are known in the art. Some embodiments of the binding domain with binding affinity to the marker/antigen cora antigen can comprise anti-cora antigen VL and VH sequence(s). Some embodiments of the binding domain with binding affinity to the marker/antigen cora antigen can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-cora antigen antibody/antibodies. Some embodiments of the binding domain with binding affinity to the marker/antigen cora antigen can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for cora antigen can have a K_{d} value of greater than 10⁻⁷ to 10⁻¹⁰ M, as determined using an in vitro binding assay.

### Anti-CD7 Binding Domains

In some embodiments of the compositions of this disclosure, the binding domain can have specific binding affinity to the marker/antigen CD7. Some embodiments of the compositions of this disclosure can comprise a bispecific bioactive assembly comprising the binding domain specific for CD7 and another binding domain (e.g., having specific binding affinity to an effector cell). The binding domain can comprise VL and VH derived from a monoclonal antibody to CD7. Monoclonal antibodies to CD7 are known in the art. Some embodiments of the binding domain with binding affinity to the marker/antigen CD7 can comprise anti-CD7 VL and VH sequence(s). Some embodiments of the binding domain with binding affinity to the marker/antigen CD7 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-CD7 antibody/antibodies. Some embodiments of the binding domain with binding affinity to the marker/antigen CD7 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for CD7 can have a K_{d} value of greater than 10⁻⁷ to 10⁻¹⁰ M, as determined using an in vitro binding assay.

### Anti-CD22 Binding Domains

In some embodiments of the compositions of this disclosure, the binding domain can have specific binding affinity to the marker/antigen CD22. Some embodiments of the compositions of this disclosure can comprise a bispecific bioactive assembly comprising the binding domain specific for CD22 and another binding domain (e.g., having specific binding affinity to an effector cell). The binding domain can comprise VL and VH derived from a monoclonal antibody to CD22. Monoclonal antibodies to CD22 are known in the art. Some embodiments of the binding domain with binding affinity to the marker/antigen CD22 can comprise anti-CD22 VL and VH sequence(s). Some embodiments of the binding domain with binding affinity to the marker/antigen CD22 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-CD22 antibody/antibodies. Some embodiments of the binding domain with binding affinity to the marker/antigen CD22 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for CD22 can have a K_{d} value of greater than 10⁻⁷ to 10⁻¹⁰ M, as determined using an in vitro binding assay.

### Anti-CD79a Binding Domains

In some embodiments of the compositions of this disclosure, the binding domain can have specific binding affinity to the marker/antigen CD79a. Some embodiments of the compositions of this disclosure can comprise a bispecific bioactive assembly comprising the binding domain specific for CD79a and another binding domain (e.g., having specific binding affinity to an effector cell). The binding domain can comprise VL and VH derived from a monoclonal antibody to CD79a. Monoclonal antibodies to CD79a are known in the art. Some embodiments of the binding domain with binding affinity to the marker/antigen CD79a can comprise anti-CD79a VL and VH sequence(s). Some embodiments of the binding domain with binding affinity to the marker/antigen CD79a can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-CD79a antibody/antibodies. Some embodiments of the binding domain with binding affinity to the marker/antigen CD79a can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for CD79a can have a K_{d} value of greater than 10⁻⁷ to 10⁻¹⁰ M, as determined using an in vitro binding assay.

### Anti-CD79b Binding Domains

In some embodiments of the compositions of this disclosure, the binding domain can have specific binding affinity to the marker/antigen CD79b. Some embodiments of the compositions of this disclosure can comprise a bispecific bioactive assembly comprising the binding domain specific for CD79b and another binding domain (e.g., having specific binding affinity to an effector cell). The binding domain can comprise VL and VH derived from a monoclonal antibody to CD79b. Monoclonal antibodies to CD79b are known in the art. Some embodiments of the binding domain with binding affinity to the marker/antigen CD79b can comprise anti-CD79b VL and VH sequence(s). Some embodiments of the binding domain with binding affinity to the marker/antigen CD79b can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-CD79b antibody/antibodies. Some embodiments of the binding domain with binding affinity to the marker/antigen CD79b can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for CD79b can have a K_{d} value of greater than 10⁻⁷ to 10⁻¹⁰ M, as determined using an in vitro binding assay.

### Anti-G250 Binding Domains

In some embodiments of the compositions of this disclosure, the binding domain can have specific binding affinity to the marker/antigen G250. Some embodiments of the compositions of this disclosure can comprise a bispecific bioactive assembly comprising the binding domain specific for G250 and another binding domain (e.g., having specific binding affinity to an effector cell). The binding domain can comprise VL and VH derived from a monoclonal antibody to G250. Monoclonal antibodies to G250 are known in the art. Some embodiments of the binding domain with binding affinity to the marker/antigen G250 can comprise anti-G250 VL and VH sequence(s). Some embodiments of the binding domain with binding affinity to the marker/antigen G250 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-G250 antibody/antibodies. Some embodiments of the binding domain with binding affinity to the marker/antigen G250 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for G250 can have a K_{d} value of greater than 10⁻⁷ to 10⁻¹⁰ M, as determined using an in vitro binding assay.

### Anti-MT-MMPs Binding Domains

In some embodiments of the compositions of this disclosure, the binding domain can have specific binding affinity to the marker/antigen MT-MMPs. Some embodiments of the compositions of this disclosure can comprise a bispecific bioactive assembly comprising the binding domain specific for MT-MMPs and another binding domain (e.g., having specific binding affinity to an effector cell). The binding domain can comprise VL and VH derived from a monoclonal antibody to MT-MMPs. Monoclonal antibodies to MT-MMPs are known in the art. Some embodiments of the binding domain with binding affinity to the marker/antigen MT-MMPs can comprise anti-MT-MMPs VL and VH sequence(s). Some embodiments of the binding domain with binding affinity to the marker/antigen MT-MMPs can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-MT-MMPs antibody/antibodies. Some embodiments of the binding domain with binding affinity to the marker/antigen MT-MMPs can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for MT-MMPs can have a K_{d} value of greater than 10⁻⁷ to 10⁻¹⁰ M, as determined using an in vitro binding assay.

### Anti-F19 Antigen Binding Domains

In some embodiments of the compositions of this disclosure, the binding domain can have specific binding affinity to the marker/antigen F19. Some embodiments of the compositions of this disclosure can comprise a bispecific bioactive assembly comprising the binding domain specific for F19 and another binding domain (e.g., having specific binding affinity to an effector cell). The binding domain can comprise VL and VH derived from a monoclonal antibody to F19. Monoclonal antibodies to F19 are known in the art. Some embodiments of the binding domain with binding affinity to the marker/antigen F19 can comprise anti-F19 VL and VH sequence(s). Some embodiments of the binding domain with binding affinity to the marker/antigen F19 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of anti-F19 antibody/antibodies. Some embodiments of the binding domain with binding affinity to the marker/antigen F19 can comprise the CDR-L1 region, the CDR-L2 region, the CDR-L3 region, the CDR-H1 region, the CDR-H2 region, and the CDR-H3 region, wherein each can be derived from the respective VL and VH sequence(s). In some embodiments of the compositions of this disclosure, the binding domain specific for F19 can have a K_{d} value of greater than 10⁻⁷ to 10⁻¹⁰ M, as determined using an in vitro binding assay.

### Anti-EphA2 Receptor Binding Domains:

In some embodiments of the compositions of this disclosure, the binding domain can have specific binding affinity to the marker/antigen EphA2. Some embodiments of the compositions of this disclosure can comprise a bispecific bioactive assembly comprising the binding domain specific for EphA2 and another binding domain (e.g., having specific binding affinity to an effector cell). The binding domain can comprise VL and VH derived from a monoclonal antibody to EphA2. Monoclonal antibodies to EphA2 are known in the art. Exemplary, non-limiting example(s) of EphA2 monoclonal antibodies and the VL and VH sequences thereof are presented in Table 6. Some embodiments of the binding domain with binding affinity to the marker/antigen EphA2 can comprise anti-EphA2 VL and VH sequence(s) set forth in Table 6. Some embodiments of the binding domain with binding affinity to the marker/antigen EphA2 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-EphA2 antibody/antibodies of Table 6. Some embodiments of the binding domain with binding affinity to the marker/antigen EphA2 can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for EphA2 can have a K_{d} value of greater than 10⁻⁷ to 10⁻¹⁰ M, as determined using an in vitro binding assay.

### Anti-Alpha 4 Integrin Binding Domains:

In some embodiments of the compositions of this disclosure, the binding domain can have specific binding affinity to the marker/antigen alpha 4 integrin. Some embodiments of the compositions of this disclosure can comprise a bispecific bioactive assembly comprising the binding domain specific for alpha 4 integrin and another binding domain (e.g., having specific binding affinity to an effector cell). The binding domain can comprise VL and VH derived from a monoclonal antibody to alpha 4 integrin. Monoclonal antibodies to alpha 4 integrin are known in the art. Exemplary, non-limiting example(s) of alpha 4 integrin monoclonal antibodies and the VL and VH sequences thereof are presented in Table 6. Some embodiments of the binding domain with binding affinity to the marker/antigen alpha 4 integrin can comprise anti-alpha 4 integrin VL and VH sequence(s) set forth in Table 6. Some embodiments of the binding domain with binding affinity to the marker/antigen alpha 4 integrin can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the natalizumab antibody of Table 6. Some embodiments of the binding domain with binding affinity to the marker/antigen alpha 4 integrin can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for alpha 4 integrin can have a K_{d} value of greater than 10⁻⁷ to 10⁻¹⁰ M, as determined using an in vitro binding assay.

### Anti-Ang2 Binding Domains:

In some embodiments of the compositions of this disclosure, the binding domain can have specific binding affinity to the marker/antigen Ang2 (Angiopoietin-2). Some embodiments of the compositions of this disclosure can comprise a bispecific bioactive assembly comprising the binding domain specific for Ang2 and another binding domain (e.g., having specific binding affinity to an effector cell). The binding domain can comprise VL and VH derived from a monoclonal antibody to Ang2. Monoclonal antibodies to Ang2 are known in the art. Exemplary, non-limiting example(s) of Ang2 monoclonal antibodies and the VL and VH sequences thereof are presented in Table 6. Some embodiments of the binding domain with binding affinity to the marker/antigen Ang2 can comprise anti-Ang2 VL and VH sequence(s) set forth in Table 6. Some embodiments of the binding domain with binding affinity to the marker/antigen Ang2 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the nesvacumab antibody of Table 6. Some embodiments of the binding domain with binding affinity to the marker/antigen Ang2 can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for Ang2 can have a K_{d} value of greater than 10⁻⁷ to 10⁻¹⁰ M, as determined using an in vitro binding assay.

### Anti-CEACAM5 Binding Domains:

In some embodiments of the compositions of this disclosure, the binding domain can have specific binding affinity to the marker/antigen CEACAM5 (Carcinoembryonic Antigen-Related Cell Adhesion Molecule 5). Some embodiments of the compositions of this disclosure can comprise a bispecific bioactive assembly comprising the binding domain specific for CEACAM5 and another binding domain (e.g., having specific binding affinity to an effector cell). The binding domain can comprise VL and VH derived from a monoclonal antibody to CEACAM5. Monoclonal antibodies to CEACAM5 are known in the art. Exemplary, non-limiting example(s) of CEACAM5 monoclonal antibodies and the VL and VH sequences thereof are presented in Table 6. Some embodiments of the binding domain with binding affinity to the marker/antigen CEACAM5 can comprise anti-CEACAM5 VL and VH sequence(s) set forth in Table 6. Some embodiments of the binding domain with binding affinity to the marker/antigen CEACAM5 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-CEACAM5 antibodies of Table 6. Some embodiments of the binding domain with binding affinity to the marker/antigen CEACAM5 can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for CEACAM5 can have a K_{d} value of greater than 10⁻⁷ to 10⁻¹⁰ M, as determined using an in vitro binding assay.

### Anti-CD38 Binding Domains:

In some embodiments of the compositions of this disclosure, the binding domain can have specific binding affinity to the marker/antigen CD38. Some embodiments of the compositions of this disclosure can comprise a bispecific bioactive assembly comprising the binding domain specific for CD38 and another binding domain (e.g., having specific binding affinity to an effector cell). The binding domain can comprise VL and VH derived from a monoclonal antibody to CD38. Monoclonal antibodies to CD38 are known in the art. Exemplary, non-limiting example(s) of CD38 monoclonal antibodies and the VL and VH sequences thereof are presented in Table 6. Some embodiments of the binding domain with binding affinity to the marker/antigen CD38 can comprise anti-CD38 VL and VH sequence(s) set forth in Table 6. Some embodiments of the binding domain with binding affinity to the marker/antigen CD38 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-CD38 antibody/antibodies of Table 6. Some embodiments of the binding domain with binding affinity to the marker/antigen CD38 can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for CD38 can have a K_{d} value of greater than 10⁻⁷ to 10⁻¹⁰ M, as determined using an in vitro binding assay.

### Anti-CD70 Binding Domains:

In some embodiments of the compositions of this disclosure, the binding domain can have specific binding affinity to the marker/antigen CD70. Some embodiments of the compositions of this disclosure can comprise a bispecific bioactive assembly comprising the binding domain specific for CD70 and another binding domain (e.g., having specific binding affinity to an effector cell). The binding domain can comprise VL and VH derived from a monoclonal antibody to CD70. Monoclonal antibodies to CD70 are known in the art. Exemplary, non-limiting example(s) of CD70 monoclonal antibodies and the VL and VH sequences thereof are presented in Table 6. Some embodiments of the binding domain with binding affinity to the marker/antigen CD70 can comprise anti-CD70 VL and VH sequence(s) set forth in Table 6. Some embodiments of the binding domain with binding affinity to the marker/antigen CD70 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-CD70 antibodies of Table 6. Some embodiments of the binding domain with binding affinity to the marker/antigen CD70 can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for CD70 can have a K_{d} value of greater than 10⁻⁷ to 10⁻¹⁰ M, as determined using an in vitro binding assay.

### Anti-cMET (Mesenchymal Epithelial Transition Factor) Binding Domains:

In some embodiments of the compositions of this disclosure, the binding domain can have specific binding affinity to the marker/antigen cMET. Some embodiments of the compositions of this disclosure can comprise a bispecific bioactive assembly comprising the binding domain specific for cMET and another binding domain (e.g., having specific binding affinity to an effector cell). The binding domain can comprise VL and VH derived from a monoclonal antibody to cMET. Monoclonal antibodies to cMET are known in the art. Exemplary, non-limiting example(s) of cMET monoclonal antibodies and the VL and VH sequences thereof are presented in Table 6. Some embodiments of the binding domain with binding affinity to the marker/antigen cMET can comprise anti-cMET VL and VH sequence(s) set forth in Table 6. Some embodiments of the binding domain with binding affinity to the marker/antigen cMET can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-cMET antibodies of Table 6. Some embodiments of the binding domain with binding affinity to the marker/antigen cMET can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for cMET can have a K_{d} value of greater than 10⁻⁷ to 10⁻¹⁰ M, as determined using an in vitro binding assay.

### Anti-CTLA4 Binding Domains:

In some embodiments of the compositions of this disclosure, the binding domain can have specific binding affinity to the marker/antigen CTLA4. Some embodiments of the compositions of this disclosure can comprise a bispecific bioactive assembly comprising the binding domain specific for CTLA4 and another binding domain (e.g., having specific binding affinity to an effector cell). The binding domain can comprise VL and VH derived from a monoclonal antibody to CTLA4. Monoclonal antibodies to CTLA4 are known in the art. Exemplary, non-limiting example(s) of CTLA4 monoclonal antibodies and the VL and VH sequences thereof are presented in Table 6. Some embodiments of the binding domain with binding affinity to the marker/antigen CTLA4 can comprise anti-CTLA4 VL and VH sequence(s) set forth in Table 6. Some embodiments of the binding domain with binding affinity to the marker/antigen CTLA4 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-CTLA4 antibodies of Table 6. Some embodiments of the binding domain with binding affinity to the marker/antigen CTLA4 can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for CTLA4 can have a K_{d} value of greater than 10⁻⁷ to 10⁻¹⁰ M, as determined using an in vitro binding assay.

### Anti-ENPP3 Binding Domains:

In some embodiments of the compositions of this disclosure, the binding domain can have specific binding affinity to the marker/antigen ENPP3 (ectonucleotide pyrophosphatase/phosphodiesterase 3). Some embodiments of the compositions of this disclosure can comprise a bispecific bioactive assembly comprising the binding domain specific for ENPP3 and another binding domain (e.g., having specific binding affinity to an effector cell). The binding domain can comprise VL and VH derived from a monoclonal antibody to ENPP3. Monoclonal antibodies to ENPP3 are known in the art. Exemplary, non-limiting example(s) of ENPP3 monoclonal antibodies and the VL and VH sequences thereof are presented in Table 6. Some embodiments of the binding domain with binding affinity to the marker/antigen ENPP3 can comprise anti-ENPP3 VL and VH sequence(s) set forth in Table 6. Some embodiments of the binding domain with binding affinity to the marker/antigen ENPP3 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the H16-7.8 antibody of Table 6. Some embodiments of the binding domain with binding affinity to the marker/antigen ENPP3 can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for ENPP3 can have a K_{d} value of greater than 10⁻⁷ to 10⁻¹⁰ M, as determined using an in vitro binding assay.

### Anti-FOLR1 Binding Domains:

In some embodiments of the compositions of this disclosure, the binding domain can have specific binding affinity to the marker/antigen FOLR1. Some embodiments of the compositions of this disclosure can comprise a bispecific bioactive assembly comprising the binding domain specific for FOLR1 and another binding domain (e.g., having specific binding affinity to an effector cell). The binding domain can comprise VL and VH derived from a monoclonal antibody to FOLR1. Monoclonal antibodies to FOLR1 are known in the art. Exemplary, non-limiting example(s) of FOLR1 monoclonal antibodies and the VL and VH sequences thereof are presented in Table 6. Some embodiments of the binding domain with binding affinity to the marker/antigen FOLR1 can comprise anti-FOLR1 VL and VH sequence(s) set forth in Table 6. Some embodiments of the binding domain with binding affinity to the marker/antigen FOLR1 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-FOLR1 antibody/antibodies of Table 6. Some embodiments of the binding domain with binding affinity to the marker/antigen FOLR1 can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for FOLR1 can have a K_{d} value of greater than 10⁻⁷ to 10⁻¹⁰ M, as determined using an in vitro binding assay.

### Anti-GPC3 Binding Domains:

In some embodiments of the compositions of this disclosure, the binding domain can have specific binding affinity to the marker/antigen GPC3 (glypican 3). Some embodiments of the compositions of this disclosure can comprise a bispecific bioactive assembly comprising the binding domain specific for GPC3 and another binding domain (e.g., having specific binding affinity to an effector cell). The binding domain can comprise VL and VH derived from a monoclonal antibody to GPC3. Monoclonal antibodies to GPC3 are known in the art. Exemplary, non-limiting example(s) of GPC3 monoclonal antibodies and the VL and VH sequences thereof are presented in Table 6. Some embodiments of the binding domain with binding affinity to the marker/antigen GPC3 can comprise anti-GPC3 VL and VH sequence(s) set forth in Table 6. Some embodiments of the binding domain with binding affinity to the marker/antigen GPC3 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-GPC3 antibody/antibodies of Table 6. Some embodiments of the binding domain with binding affinity to the marker/antigen GPC3 can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for GPC3 can have a K_{d} value of greater than 10⁻⁷ to 10⁻¹⁰ M, as determined using an in vitro binding assay.

### Anti-PD-L1 Binding Domains:

In some embodiments of the compositions of this disclosure, the binding domain can have specific binding affinity to the marker/antigen PD-L1. Some embodiments of the compositions of this disclosure can comprise a bispecific bioactive assembly comprising the binding domain specific for PD-L1 and another binding domain (e.g., having specific binding affinity to an effector cell). The binding domain can comprise VL and VH derived from a monoclonal antibody to PD-L1. Monoclonal antibodies to PD-L1 are known in the art. Exemplary, non-limiting example(s) of PD-L1 monoclonal antibodies and the VL and VH sequences thereof are presented in Table 6. Some embodiments of the binding domain with binding affinity to the marker/antigen PD-L1 can comprise anti-PD-L1 VL and VH sequence(s) set forth in Table 6. Some embodiments of the binding domain with binding affinity to the marker/antigen PD-L1 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-PD-L1 antibody/antibodies of Table 6. Some embodiments of the binding domain with binding affinity to the marker/antigen PD-L1 can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for PD-L1 can have a K_{d} value of greater than 10⁻⁷ to 10⁻¹⁰ M, as determined using an in vitro binding assay.

### Anti-ROR1 Binding Domains:

In some embodiments of the compositions of this disclosure, the binding domain can have specific binding affinity to the marker/antigen ROR1. Some embodiments of the compositions of this disclosure can comprise a bispecific bioactive assembly comprising the binding domain specific for ROR1 and another binding domain (e.g., having specific binding affinity to an effector cell). The binding domain can comprise VL and VH derived from a monoclonal antibody to ROR1. Monoclonal antibodies to ROR1 are known in the art. Exemplary, non-limiting example(s) of ROR1 monoclonal antibodies and the VL and VH sequences thereof are presented in Table 6. Some embodiments of the binding domain with binding affinity to the marker/antigen ROR1 can comprise anti-ROR1 VL and VH sequence(s) set forth in Table 6. Some embodiments of the binding domain with binding affinity to the marker/antigen ROR1 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-ROR1 antibody/antibodies of Table 6. Some embodiments of the binding domain with binding affinity to the marker/antigen ROR1 can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for ROR1 can have a K_{d} value of greater than 10⁻⁷ to 10⁻¹⁰ M, as determined using an in vitro binding assay.

### Anti-TPBG/5T4 Binding Domains:

In some embodiments of the compositions of this disclosure, the binding domain can have specific binding affinity to the marker/antigen TPBG/5T4 (trophoblast glycoprotein). Some embodiments of the compositions of this disclosure can comprise a bispecific bioactive assembly comprising the binding domain specific for TPBG/5T4 and another binding domain (e.g., having specific binding affinity to an effector cell). The binding domain can comprise VL and VH derived from a monoclonal antibody to TPBG/5T4. Monoclonal antibodies to TPBG/5T4 are known in the art. Exemplary, non-limiting example(s) of TPBG/5T4 monoclonal antibodies and the VL and VH sequences thereof are presented in Table 6. Some embodiments of the binding domain with binding affinity to the marker/antigen TPBG/5T4 can comprise anti-TPBG/5T4 VL and VH sequence(s) set forth in Table 6. Some embodiments of the binding domain with binding affinity to the marker/antigen TPBG/5T4 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-TPBG/5T4 antibody/antibodies of Table 6. Some embodiments of the binding domain with binding affinity to the marker/antigen TPBG/5T4 can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for TPBG/5T4 can have a K_{d} value of greater than 10⁻⁷ to 10⁻¹⁰ M, as determined using an in vitro binding assay.

### Anti-TROP-2 Binding Domains:

In some embodiments of the compositions of this disclosure, the binding domain can have specific binding affinity to the marker/antigen TROP-2. Some embodiments of the compositions of this disclosure can comprise a bispecific bioactive assembly comprising the binding domain specific for TROP-2 and another binding domain (e.g., having specific binding affinity to an effector cell). The binding domain can comprise VL and VH derived from a monoclonal antibody to TROP-2. Monoclonal antibodies to TROP-2 are known in the art. Exemplary, non-limiting example(s) of TROP-2 monoclonal antibodies and the VL and VH sequences thereof are presented in Table 6. Some embodiments of the binding domain with binding affinity to the marker/antigen TROP-2 can comprise anti-TROP-2 VL and VH sequence(s) set forth in Table 6. Some embodiments of the binding domain with binding affinity to the marker/antigen TROP-2 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-TROP-2 antibody/antibodies of Table 6. Some embodiments of the binding domain with binding affinity to the marker/antigen TROP-2 can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for TROP-2 can have a K_{d} value of greater than 10⁻⁷ to 10⁻¹⁰ M, as determined using an in vitro binding assay.

### Anti-VEGFR1 Binding Domains:

In some embodiments of the compositions of this disclosure, the binding domain can have specific binding affinity to the marker/antigen VEGFR1. Some embodiments of the compositions of this disclosure can comprise a bispecific bioactive assembly comprising the binding domain specific for VEGFR1 and another binding domain (e.g., having specific binding affinity to an effector cell). The binding domain can comprise VL and VH derived from a monoclonal antibody to VEGFR1. Monoclonal antibodies to VEGFR1 are known in the art. Exemplary, non-limiting example(s) of VEGFR1 monoclonal antibodies and the VL and VH sequences thereof are presented in Table 6. Some embodiments of the binding domain with binding affinity to the marker/antigen VEGFR1 can comprise anti-VEGFR1 VL and VH sequence(s) set forth in Table 6. Some embodiments of the binding domain with binding affinity to the marker/antigen VEGFR1 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-VEGFR1 antibody/antibodies of Table 6. Some embodiments of the binding domain with binding affinity to the marker/antigen VEGFR1 can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for VEGFR1 can have a K_{d} value of greater than 10⁻⁷ to 10⁻¹⁰ M, as determined using an in vitro binding assay.

### Anti-VEGFR2 Binding Domains:

In some embodiments of the compositions of this disclosure, the binding domain can have specific binding affinity to the marker/antigen VEGFR2 (vascular endothelial growth factor receptor 2). Some embodiments of the compositions of this disclosure can comprise a bispecific bioactive assembly comprising the binding domain specific for VEGFR2 and another binding domain (e.g., having specific binding affinity to an effector cell). The binding domain can comprise VL and VH derived from a monoclonal antibody to VEGFR2. Monoclonal antibodies to VEGFR2 are known in the art. Exemplary, non-limiting example(s) of VEGFR2 monoclonal antibodies and the VL and VH sequences thereof are presented in Table 6. Some embodiments of the binding domain with binding affinity to the marker/antigen VEGFR2 can comprise anti-VEGFR2 VL and VH sequence(s) set forth in Table 6. Some embodiments of the binding domain with binding affinity to the marker/antigen VEGFR2 can comprise VH and VL regions wherein each VH and VL regions can exhibit at least (about) 90%, or at least (about) 91%, or at least (about) 92%, or at least (about) 93%, or at least (about) 94%, or at least (about) 95%, or at least (about) 96%, or at least (about) 97%, or at least (about) 98%, or at least (about) 99% identity to, or is identical to, paired VL and VH sequence(s) of the anti-VEGFR2 antibodies of Table 6. Some embodiments of the binding domain with binding affinity to the marker/antigen VEGFR2 can comprise the CDR-L1 region, the CDR-L2 region, the CDR-L3 region, the CDR-H1 region, the CDR-H2 region, and the CDR-H3 region, wherein each can be derived from the respective VL and VH sequence(s) set forth in Table 6. In some embodiments of the compositions of this disclosure, the binding domain specific for VEGFR2 can have a K_{d} value of greater than 10⁻⁷ to 10⁻¹⁰ M, as determined using an in vitro binding assay.

It is specifically contemplated that the compositions of this disclosure can comprise any one of the foregoing binding domains or sequence variants thereof so long as the variants exhibit binding specificity for the described antigen. A sequence variant can be created by substitution of an amino acid in the VL or VH sequence with a different amino acid. In deletion variants, one or more amino acid residues in a VL or VH sequence as described herein are removed. Deletion variants, therefore, include all fragments of a binding domain polypeptide sequence. In substitution variants, one or more amino acid residues of a VL or VH (or CDR) polypeptide are removed and replaced with alternative residues. The substitutions can be conservative in nature and conservative substitutions of this type are well known in the art. In addition, it is specifically contemplated that the compositions comprising the first and the second binding domains disclosed herein can be utilized in any of the methods disclosed herein.

### Exemplary Activatable Therapeutic Agents

In some embodiments of the compositions of this disclosure, the activatable therapeutic agent is a recombinant polypeptide comprising an amino acid sequence having at least (about) 80% sequence identity to a sequence set forth in Table 7, or a subset thereof. The activatable therapeutic agent can comprise an amino acid sequence having at least (about) 81%, at least (about) 82%, at least (about) 83%, at least (about) 84%, at least (about) 85%, at least (about) 86%, at least (about) 87%, at least (about) 88%, at least (about) 89%, at least (about) 90%, at least (about) 91%, at least (about) 92%, at least (about) 93%, at least (about) 94%, at least (about) 95%, at least (about) 96%, at least (about) 97%, at least (about) 98%, or at least (about) 99% sequence identity to a sequence set forth in Table 7, or a subset thereof. The activatable therapeutic agent can comprise an amino acid sequence identical to a sequence set forth in Table 7, or a subset thereof. It is specifically contemplated that the compositions of this disclosure can comprise sequence variants of the amino acid sequences set forth in Table 7, or a subset thereof, such as with linker sequence(s) inserted or with purification tag sequence(s) attached thereto, so long as the variants exhibit substantially similar or same bioactivity/bioactivities and/or activation mechanism(s).

TABLE 7
Amino acid sequences of exemplary recombinant polypeptides

| SEQ ID | |
|---|---|
| NOS. | Amino Acid Sequence |
| 9 | ASHHHHHHSPAGSPTSTEEGTSESATPESGPGTSTEPSEGSAPGTSESATPESGPGS |
| | EPATSGSETPGTSESATPESGPGSEPATSGSETPGTSESATPESGPGTSTEPSEGSAP |
| | GSPAGSPTSTEEGTSESATPESGPGSEPATSGSETPGTSESATPESGPGSPAGSPTST |
| | EEGSPAGSPTSTEEGTSTEPSEGSAPGTSESATPESGPGTSESATPESGPGTSESATP |
| | ESGPGSEPATSGSETPGSEPATSGSETPGSPAGSPTSTEEGTSTEPSEGSAPGTSTEP |
| | SEGSAPGGSAPGPSGHMGRATSGSETPGTDIQMTQSPSSLSASVGDRVTITCKASQ |
| | DVSIGVAWYQQKPGKAPKLLIYSASYRYTGVPSRFSGSGSGTDFTLTISSLQPEDF |
| | ATYYCQQYYTYPYTFGQGTKVEIKGATPPETGAETESPGETTGGSAESEPPGEGE |
| | VQLVESGGGLVQPGGSLRLSCAASGFTFTDYTMDWVRQAPGKGLEWVADVNPN |
| | SGGSIYNQRFKGRFTLSVDRSKNTLYLQMNSLRAEDTAVYYCARNLGPSFYFDY |
| | WGQGTLVTVSSGGGGSELVVTQEPSLTVSPGGTVTLTCRSSNGAVTSSNYANWV |
| | QQKPGQAPRGLIGGTNKRAPGTPARFSGSLLGGKAALTLSGVQPEDEAVYYCAL |
| | WYPNLWVFGGGTKLTVLGATPPETGAETESPGETTGGSAESEPPGEGEVQLLES |
| | GGGIVQPGGSLKLSCAASGFTFNTYAMNWVRQAPGKGLEWVARIRSKYNNYAT |
| | YYADSVKDRFTISRDDSKNTVYLQMNNLKTEDTAVYYCVRHENFGNSYVSWFAH |
| | WGQGTLVTVSSGTAEAASASGGPSGHMGRPGSPAGSPTSTEEGTSESATPESGPG |
| | TSTEPSEGSAPGSPAGSPTSTEEGTSTEPSEGSAPGTSTEPSEGSAPGTSESATPESG |
| | PGSEPATSGSETPGSEPATSGSETPGSPAGSPTSTEEGTSESATPESGPGTSTEPSEG |
| | SAPGTSTEPSEGSAPGSPAGSPTSTEEGTSTEPSEGSAPGTSTEPSEGSAPGTSESAT |
| | PESGPGTSTEPSEGSAPGTSESATPESGPGSEPATSGSETPGTSTEPSEGSAPGTSTE |
| | PSEGSAPGTSESATPESGPGTSESATPESGPGSPAGSPTSTEEGTSESATPESGPGSE |
| | PATSGSETPGTSESATPESGPGTSTEPSEGSAPGTSTEPSEGSAPGTSTEPSEGSAPG |
| | TSTEPSEGSAPGTSTEPSEGSAPGTSTEPSEGSAPGSPAGSPTSTEEGTSTEPSEGSA |
| | PGTSESATPESGPGSEPATSGSETPGTSESATPESGPGSEPATSGSETPGTSESATPE |
| | SGPGTSTEPSEGSAPGTSESATPESGPGSPAGSPTSTEEGSPAGSPTSTEEGSPAGSP |
| | TSTEEGTSESATPESGPGTSTEPSEGSAPGAAEPEA |
| 10 | ASHHHHHHSPAGSPTSTEEGTSESATPESGPGTSTEPSEGSAPGTSESATPESGPGS |
| | EPATSGSETPGTSESATPESGPGSEPATSGSETPGTSESATPESGPGTSTEPSEGSAP |
| | GSPAGSPTSTEEGTSESATPESGPGSEPATSGSETPGTSESATPESGPGSPAGSPTST |
| | EEGSPAGSPTSTEEGTSTEPSEGSAPGTSESATPESGPGTSESATPESGPGTSESATP |
| | ESGPGSEPATSGSETPGSEPATSGSETPGSPAGSPTSTEEGTSTEPSEGSAPGTSTEP |
| | SEGSAPGGSAPHPVELLARATSGSETPGTDIQMTQSPSSLSASVGDRVTITCQASQ |
| | DISNYLNWYQQKPGKAPKLLIYDASNLETGVPSRFSGSGSGTDFTFTISSLQPEDIA |
| | TYFCQHFDHLPLAFGGGTKVEIKGATPPETGAETESPGETTGGSAESEPPGEGQV |
| | QLQESGPGLVKPSETLSLTCTVSGGSVSSGDYYWTWIRQSPGKGLEWIGHIYYSG |
| | NTNYNPSLKSRLTISIDTSKTQFSLKLSSVTAADTAIYYCVRDRVTGAFDIWGQGT |
| | MVTVSSGGGGSELVVTQEPSLTVSPGGTVTLTCRSSTGAVTTSNYANWVQQKPG |
| | QAPRGLIGGTNKRAPGTPARFSGSLLGGKAALTLSGVQPEDEAEYYCALWYSNL |
| | WVFGGGTKLTVLGATPPETGAETESPGETTGGSAESEPPGEGEVQLLESGGGLV |
| | QPGGSLKLSCAASGFTFNTYAMNWVRQAPGKGLEWVARIRSKYNNYATYYADS |
| | VKDRFTISRDDSKNTAYLQMNNLKTEDTAVYYCVRHGNFGNSYVSWFAYWGQG |
| | TLVTVSSGTAEAASASGHPVELLARPGSPAGSPTSTEEGTSESATPESGPGTSTEPS |
| | EGSAPGSPAGSPTSTEEGTSTEPSEGSAPGTSTEPSEGSAPGTSESATPESGPGSEPA |
| | TSGSETPGSEPATSGSETPGSPAGSPTSTEEGTSESATPESGPGTSTEPSEGSAPGTS |
| | TEPSEGSAPGSPAGSPTSTEEGTSTEPSEGSAPGTSTEPSEGSAPGTSESATPESGPG |
| | TSTEPSEGSAPGTSESATPESGPGSEPATSGSETPGTSTEPSEGSAPGTSTEPSEGSA |
| | PGTSESATPESGPGTSESATPESGPGSPAGSPTSTEEGTSESATPESGPGSEPATSGS |
| | ETPGTSESATPESGPGTSTEPSEGSAPGTSTEPSEGSAPGTSTEPSEGSAPGTSTEPS |
| | EGSAPGTSTEPSEGSAPGTSTEPSEGSAPGSPAGSPTSTEEGTSTEPSEGSAPGTSES |
| | ATPESGPGSEPATSGSETPGTSESATPESGPGSEPATSGSETPGTSESATPESGPGTS |
| | TEPSEGSAPGTSESATPESGPGSPAGSPTSTEEGSPAGSPTSTEEGSPAGSPTSTEEG |
| | TSESATPESGPGTSTEPSEGSAPGAAEPEA |
| 11 | ASHHHHHHSPAGSPTSTEEGTSESATPESGPGTSTEPSEGSAPGTSESATPESGPGS |
| | EPATSGSETPGTSESATPESGPGSEPATSGSETPGTSESATPESGPGTSTEPSEGSAP |
| | GSPAGSPTSTEEGTSESATPESGPGSEPATSGSETPGTSESATPESGPGSPAGSPTST |
| | EEGSPAGSPTSTEEGTSTEPSEGSAPGTSESATPESGPGTSESATPESGPGTSESATP |
| | ESGPGSEPATSGSETPGSEPATSGSETPGSPAGSPTSTEEGTSTEPSEGSAPGTSTEP |
| | SEGSAPGGSAPVSKRFPVGATSGSETPGTDIQMTQSPSSLSASVGDRVTITCQASQ |
| | DISNYLNWYQQKPGKAPKLLIYDASNLETGVPSRFSGSGSGTDFTFTISSLQPEDIA |
| | TYFCQHFDHLPLAFGGGTKVEIKGATPPETGAETESPGETTGGSAESEPPGEGQV |
| | QLQESGPGLVKPSETLSLTCTVSGGSVSSGDYYWTWIRQSPGKGLEWIGHIYYSG |
| | NTNYNPSLKSRLTISIDTSKTQFSLKLSSVTAADTAIYYCVRDRVTGAFDIWGQGT |
| | MVTVSSGGGGSELVVTQEPSLTVSPGGTVTLTCRSSTGAVTTSNYANWVQQKPG |
| | QAPRGLIGGTNKRAPGTPARFSGSLLGGKAALTLSGVQPEDEAEYYCALWYSNL |
| | WVFGGGTKLTVLGATPPETGAETESPGETTGGSAESEPPGEGEVQLLESGGGLV |
| | QPGGSLKLSCAASGFTFNTYAMNWVRQAPGKGLEWVARIRSKYNNYATYYADS |
| | VKDRFTISRDDSKNTAYLQMNNLKTEDTAVYYCVRHGNFGNSYVSWFAYWGQG |
| | TLVTVSSGTAEAASASGVSKRFPVGPGSPAGSPTSTEEGTSESATPESGPGTSTEPS |
| | EGSAPGSPAGSPTSTEEGTSTEPSEGSAPGTSTEPSEGSAPGTSESATPESGPGSEPA |
| | TSGSETPGSEPATSGSETPGSPAGSPTSTEEGTSESATPESGPGTSTEPSEGSAPGTS |
| | TEPSEGSAPGSPAGSPTSTEEGTSTEPSEGSAPGTSTEPSEGSAPGTSESATPESGPG |
| | TSTEPSEGSAPGTSESATPESGPGSEPATSGSETPGTSTEPSEGSAPGTSTEPSEGSA |
| | PGTSESATPESGPGTSESATPESGPGSPAGSPTSTEEGTSESATPESGPGSEPATSGS |
| | ETPGTSESATPESGPGTSTEPSEGSAPGTSTEPSEGSAPGTSTEPSEGSAPGTSTEPS |
| | EGSAPGTSTEPSEGSAPGTSTEPSEGSAPGSPAGSPTSTEEGTSTEPSEGSAPGTSES |
| | ATPESGPGSEPATSGSETPGTSESATPESGPGSEPATSGSETPGTSESATPESGPGTS |
| | TEPSEGSAPGTSESATPESGPGSPAGSPTSTEEGSPAGSPTSTEEGSPAGSPTSTEEG |
| | TSESATPESGPGTSTEPSEGSAPGAAEPEA |
| 12 | ASSPAGSPTSTESGTSESATPESGPGTETEPSEGSAPGTSESATPESGPGSEPATSGS |
| | ETPGTSESATPESGPGSTPAESGSETPGTSESATPESGPGTSTEPSEGSAPGSPAGSP |
| | TSTEEGTSESATPESGPGESPATSGSTPEGTSESATPESGPGSPAGSPTSTEEGSPAG |
| | SPTSTEEGTSTEPSEGSAPGTSESATPESGPGTSESATPESGPGTSESATPESGPGSE |
| | PATSGSETPGSEPATSGSETPGSPAGSPTSTEEGTSTEPSEGSAPGTSTEPSEGSAPG |
| | GSAPEAGRSANHTPAGLTGPATSGSETPGTEIVLTQSPATLSLSPGERATLSCKAS |
| | QDVSIGVAWYQQKPGQAPRLLIYSASYRYSGVPARFSGSGSGTDFTLTISSLEPED |
| | FAVYYCQQYYIYPYTFGQGTKVEIKGATPPETGAETESPGETTGGSAESEPPGEG |
| | QVQLVQSGVEVKKPGASVKVSCKASGFTFTDYTMDWVRQAPGQGLEWMADVN |
| | PNSGGSIYNQRFKGRVTLTTDSSTTTAYMELKSLQFDDTAVYYCARNLGPSFYFD |
| | YWGQGTLVTVSSGGGSELVVTQEPSLTVSPGGTVTLTCRSSNGAVTSSNYANWV |
| | QQKPGQAPRGLIGGTNKRAPGTPARFSGSSLGGKAALTLSGVQPEDEAVYYCAL |
| | WYPNLWVFGGGTKLTVLGATPPETGAETESPGETTGGSAESEPPGEGEVQLQES |
| | GGGIVQPGGSLKLSCAASGFTFNTYAMNWVRQAPGKGLEWVARIRSKYNNYAT |
| | YYADSVKDRFTISRDDSKNTVYLQMNNLKTEDTAVYYCVRHENFGNSYVSWFAH |
| | WGQGTLVTVSSGTAEAASASGEAGRSANHTPAGLTGPTPESGPGTSESATPESGP |
| | GSPAGSPTSTEEGTSESATPESGPGSEPATSGSETPGTSESATPESGPGTSTEPSEGS |
| | APGTSTEPSEGSAPGTSTEPSEGSAPGTSTEPSEGSAPGTSTEPSEGSAPGTSTEPSE |
| | GSAPGSPAGSPTSTEEGTSTEPSEGSAPGTSESATPESGPGSEPATSGSETPGTSESA |
| | TPESGPGSEPATSGSETPGTSESATPESGPGTSTEPSEGSAPGTSESATPESGPGSPA |
| | GSPTSTEEGSPAGSPTSTEEGSPAGSPTSTEEGTSESATPESGPGTSTEPSEGSAPGT |
| | SESATPESGPGSEPATSGSETPGTSESATPESGPGSEPATSGSETPGTSESATPESGP |
| | GTSTEPSEGSAPGSPAGSPTSTEEGTSESATPESGPGSEPATSGSETPGTSESATPES |
| | GPGSPAGSPTSTEEGSPAGSPTSTEEGTSTEPSEGSAPGTSESATPESGPGTSESATP |
| | ESGPGTSESATPESGPGSEPATSGSETPGSESATSGSETPGSPAGSPTSTEEGTSTEP |
| | SEGSAPGTSTEPSEGSAPGSEPATSGSETPGTSESA |
| 13 | ASHHHHHHSPAGSPTSTEEGTSESATPESGPGTSTEPSEGSAPGTSESATPESGPGS |
| | EPATSGSETPGTSESATPESGPGSEPATSGSETPGTSESATPESGPGTSTEPSEGSAP |
| | GSPAGSPTSTEEGTSESATPESGPGSEPATSGSETPGTSESATPESGPGSPAGSPTST |
| | EEGSPAGSPTSTEEGTSTEPSEGSAPGTSESATPESGPGTSESATPESGPGTSESATP |
| | ESGPGSEPATSGSETPGSEPATSGSETPGSPAGSPTSTEEGTSTEPSEGSAPGTSTEP |
| | SEGSAPGGSAPSPEAQAAAATSGSETPGTDIQMTQSPSSLSASVGDRVTITCQASQ |
| | DISNYLNWYQQKPGKAPKLLIYDASNLETGVPSRFSGSGSGTDFTFTISSLQPEDIA |
| | TYFCQHFDHLPLAFGGGTKVEIKGATPPETGAETESPGETTGGSAESEPPGEGQV |
| | QLQESGPGLVKPSETLSLTCTVSGGSVSSGDYYWTWIRQSPGKGLEWIGHIYYSG |
| | NTNYNPSLKSRLTISIDTSKTQFSLKLSSVTAADTAIYYCVRDRVTGAFDIWGQGT |
| | MVTVSSGGGGSELVVTQEPSLTVSPGGTVTLTCRSSTGAVTTSNYANWVQQKPG |
| | QAPRGLIGGTNKRAPGTPARFSGSLLGGKAALTLSGVQPEDEAEYYCALWYSNL |
| | WVFGGGTKLTVLGATPPETGAETESPGETTGGSAESEPPGEGEVQLLESGGGLV |
| | QPGGSLKLSCAASGFTFNTYAMNWVRQAPGKGLEWVARIRSKYNNYATYYADS |
| | VKDRFTISRDDSKNTAYLQMNNLKTEDTAVYYCVRHGNFGNSYVSWFAYWGQG |
| | TLVTVSSGTAEAASASGSPEAQAAAPGSPAGSPTSTEEGTSESATPESGPGTSTEPS |
| | EGSAPGSPAGSPTSTEEGTSTEPSEGSAPGTSTEPSEGSAPGTSESATPESGPGSEPA |
| | TSGSETPGSEPATSGSETPGSPAGSPTSTEEGTSESATPESGPGTSTEPSEGSAPGTS |
| | TEPSEGSAPGSPAGSPTSTEEGTSTEPSEGSAPGTSTEPSEGSAPGTSESATPESGPG |
| | TSTEPSEGSAPGTSESATPESGPGSEPATSGSETPGTSTEPSEGSAPGTSTEPSEGSA |
| | PGTSESATPESGPGTSESATPESGPGSPAGSPTSTEEGTSESATPESGPGSEPATSGS |
| | ETPGTSESATPESGPGTSTEPSEGSAPGTSTEPSEGSAPGTSTEPSEGSAPGTSTEPS |
| | EGSAPGTSTEPSEGSAPGTSTEPSEGSAPGSPAGSPTSTEEGTSTEPSEGSAPGTSES |
| | ATPESGPGSEPATSGSETPGTSESATPESGPGSEPATSGSETPGTSESATPESGPGTS |
| | TEPSEGSAPGTSESATPESGPGSPAGSPTSTEEGSPAGSPTSTEEGSPAGSPTSTEEG |
| | TSESATPESGPGTSTEPSEGSAPGAAEPEA |
| 14 | ASHHHHHHSPAGSPTSTEEGTSESATPESGPGTSTEPSEGSAPGTSESATPESGPGS |
| | EPATSGSETPGTSESATPESGPGSEPATSGSETPGTSESATPESGPGTSTEPSEGSAP |
| | GSPAGSPTSTEEGTSESATPESGPGSEPATSGSETPGTSESATPESGPGSPAGSPTST |
| | EEGSPAGSPTSTEEGTSTEPSEGSAPGTSESATPESGPGTSESATPESGPGTSESATP |
| | ESGPGSEPATSGSETPGSEPATSGSETPGSPAGSPTSTEEGTSTEPSEGSAPGTSTEP |
| | SEGSAPGGSAPEAGRSANHGVRGLTGPATSGSETPGTDIQMTQSPSSLSASVGDRV |
| | TITCKASQDVSIGVAWYQQKPGKAPKLLIYSASYRYTGVPSRFSGSGSGTDFTLTI |
| | SSLQPEDFATYYCQQYYTYPYTFGQGTKVEIKGATPPETGAETESPGETTGGSAES |
| | EPPGEGEVQLVESGGGLVQPGGSLRLSCAASGFTFTDYTMDWVRQAPGKGLEW |
| | VADVNPNSGGSIYNQRFKGRFTLSVDRSKNTLYLQMNSLRAEDTAVYYCARNLG |
| | PSFYFDYWGQGTLVTVSSGGGGSELVVTQEPSLTVSPGGTVTLTCRSSNGAVTSS |
| | NYANWVQQKPGQAPRGLIGGTNKRAPGTPARFSGSLLGGKAALTLSGVQPEDEA |
| | VYYCALWYPNLWVFGGGTKLTVLGATPPETGAETESPGETTGGSAESEPPGEGE |
| | VQLLESGGGIVQPGGSLKLSCAASGFTFNTYAMNWVRQAPGKGLEWVARIRSKY |
| | NNYATYYADSVKDRFTISRDDSKNTVYLQMNNLKTEDTAVYYCVRHENFGNSYV |
| | SWFAHWGQGTLVTVSSGTAEAASASGEAGRSANHGVRGLTGPPGSPAGSPTSTE |
| | EGTSESATPESGPGTSTEPSEGSAPGSPAGSPTSTEEGTSTEPSEGSAPGTSTEPSEG |
| | SAPGTSESATPESGPGSEPATSGSETPGSEPATSGSETPGSPAGSPTSTEEGTSESAT |
| | PESGPGTSTEPSEGSAPGTSTEPSEGSAPGSPAGSPTSTEEGTSTEPSEGSAPGTSTE |
| | PSEGSAPGTSESATPESGPGTSTEPSEGSAPGTSESATPESGPGSEPATSGSETPGTS |
| | TEPSEGSAPGTSTEPSEGSAPGTSESATPESGPGTSESATPESGPGSPAGSPTSTEEG |
| | TSESATPESGPGSEPATSGSETPGTSESATPESGPGTSTEPSEGSAPGTSTEPSEGSA |
| | PGTSTEPSEGSAPGTSTEPSEGSAPGTSTEPSEGSAPGTSTEPSEGSAPGSPAGSPTS |
| | TEEGTSTEPSEGSAPGTSESATPESGPGSEPATSGSETPGTSESATPESGPGSEPATS |
| | GSETPGTSESATPESGPGTSTEPSEGSAPGTSESATPESGPGSPAGSPTSTEEGSPAG |
| | SPTSTEEGSPAGSPTSTEEGTSESATPESGPGTSTEPSEGSAPGAAEPEA |
| 15 | ASHHHHHHSPAGSPTSTEEGTSESATPESGPGTSTEPSEGSAPGTSESATPESGPGS |
| | EPATSGSETPGTSESATPESGPGSEPATSGSETPGTSESATPESGPGTSTEPSEGSAP |
| | GSPAGSPTSTEEGTSESATPESGPGSEPATSGSETPGTSESATPESGPGSPAGSPTST |
| | EEGSPAGSPTSTEEGTSTEPSEGSAPGTSESATPESGPGTSESATPESGPGTSESATP |
| | ESGPGSEPATSGSETPGSEPATSGSETPGSPAGSPTSTEEGTSTEPSEGSAPGTSTEP |
| | SEGSAPGGSAPLTSDLQAQATSGSETPGTDIQMTQSPSSLSASVGDRVTITCKASQ |
| | DVSIGVAWYQQKPGKAPKLLIYSASYRYTGVPSRFSGSGSGTDFTLTISSLQPEDF |
| | ATYYCQQYYTYPYTFGQGTKVEIKGATPPETGAETESPGETTGGSAESEPPGEGE |
| | VQLVESGGGLVQPGGSLRLSCAASGFTFTDYTMDWVRQAPGKGLEWVADVNPN |
| | SGGSIYNQRFKGRFTLSVDRSKNTLYLQMNSLRAEDTAVYYCARNLGPSFYFDY |
| | WGQGTLVTVSSGGGGSELVVTQEPSLTVSPGGTVTLTCRSSNGAVTSSNYANWV |
| | QQKPGQAPRGLIGGTNKRAPGTPARFSGSLLGGKAALTLSGVQPEDEAVYYCAL |
| | WYPNLWVFGGGTKLTVLGATPPETGAETESPGETTGGSAESEPPGEGEVQLLES |
| | GGGIVQPGGSLKLSCAASGFTFNTYAMNWVRQAPGKGLEWVARIRSKYNNYAT |
| | YYADSVKDRFTISRDDSKNTVYLQMNNLKTEDTAVYYCVRHENFGNSYVSWFAH |
| | WGQGTLVTVSSGTAEAASASGLTSDLQAQPGSPAGSPTSTEEGTSESATPESGPGT |
| | STEPSEGSAPGSPAGSPTSTEEGTSTEPSEGSAPGTSTEPSEGSAPGTSESATPESGP |
| | GSEPATSGSETPGSEPATSGSETPGSPAGSPTSTEEGTSESATPESGPGTSTEPSEGS |
| | APGTSTEPSEGSAPGSPAGSPTSTEEGTSTEPSEGSAPGTSTEPSEGSAPGTSESATP |
| | ESGPGTSTEPSEGSAPGTSESATPESGPGSEPATSGSETPGTSTEPSEGSAPGTSTEP |
| | SEGSAPGTSESATPESGPGTSESATPESGPGSPAGSPTSTEEGTSESATPESGPGSEP |
| | ATSGSETPGTSESATPESGPGTSTEPSEGSAPGTSTEPSEGSAPGTSTEPSEGSAPGT |
| | STEPSEGSAPGTSTEPSEGSAPGTSTEPSEGSAPGSPAGSPTSTEEGTSTEPSEGSAP |
| | GTSESATPESGPGSEPATSGSETPGTSESATPESGPGSEPATSGSETPGTSESATPES |
| | GPGTSTEPSEGSAPGTSESATPESGPGSPAGSPTSTEEGSPAGSPTSTEEGSPAGSPT |
| | STEEGTSESATPESGPGTSTEPSEGSAPGAAEPEA |
| 16 | ASHHHHHHSPAGSPTSTEEGTSESATPESGPGTSTEPSEGSAPGTSESATPESGPGS |
| | EPATSGSETPGTSESATPESGPGSEPATSGSETPGTSESATPESGPGTSTEPSEGSAP |
| | GSPAGSPTSTEEGTSESATPESGPGSEPATSGSETPGTSESATPESGPGSPAGSPTST |
| | EEGSPAGSPTSTEEGTSTEPSEGSAPGTSESATPESGPGTSESATPESGPGTSESATP |
| | ESGPGSEPATSGSETPGSEPATSGSETPGSPAGSPTSTEEGTSTEPSEGSAPGTSTEP |
| | SEGSAPGGSAPQPVSLANTATSGSETPGTDIQMTQSPSSLSASVGDRVTITCKASQD |
| | VSIGVAWYQQKPGKAPKLLIYSASYRYTGVPSRFSGSGSGTDFTLTISSLQPEDFA |
| | TYYCQQYYTYPYTFGQGTKVEIKGATPPETGAETESPGETTGGSAESEPPGEGEV |
| | QLVESGGGLVQPGGSLRLSCAASGFTFTDYTMDWVRQAPGKGLEWVADVNPNS |
| | GGSIYNQRFKGRFTLSVDRSKNTLYLQMNSLRAEDTAVYYCARNLGPSFYFDYW |
| | GQGTLVTVSSGGGGSELVVTQEPSLTVSPGGTVTLTCRSSNGAVTSSNYANWVQ |
| | QKPGQAPRGLIGGTNKRAPGTPARFSGSLLGGKAALTLSGVQPEDEAVYYCALW |
| | YPNLWVFGGGTKLTVLGATPPETGAETESPGETTGGSAESEPPGEGEVQLLESGG |
| | GIVQPGGSLKLSCAASGFTFNTYAMNWVRQAPGKGLEWVARIRSKYNNYATYY |
| | ADSVKDRFTISRDDSKNTVYLQMNNLKTEDTAVYYCVRHENFGNSYVSWFAHW |
| | GQGTLVTVSSGTAEAASASGQPVSLANTPGSPAGSPTSTEEGTSESATPESGPGTST |
| | EPSEGSAPGSPAGSPTSTEEGTSTEPSEGSAPGTSTEPSEGSAPGTSESATPESGPGS |
| | EPATSGSETPGSEPATSGSETPGSPAGSPTSTEEGTSESATPESGPGTSTEPSEGSAP |
| | GTSTEPSEGSAPGSPAGSPTSTEEGTSTEPSEGSAPGTSTEPSEGSAPGTSESATPES |
| | GPGTSTEPSEGSAPGTSESATPESGPGSEPATSGSETPGTSTEPSEGSAPGTSTEPSE |
| | GSAPGTSESATPESGPGTSESATPESGPGSPAGSPTSTEEGTSESATPESGPGSEPAT |
| | SGSETPGTSESATPESGPGTSTEPSEGSAPGTSTEPSEGSAPGTSTEPSEGSAPGTST |
| | EPSEGSAPGTSTEPSEGSAPGTSTEPSEGSAPGSPAGSPTSTEEGTSTEPSEGSAPGT |
| | SESATPESGPGSEPATSGSETPGTSESATPESGPGSEPATSGSETPGTSESATPESGP |
| | GTSTEPSEGSAPGTSESATPESGPGSPAGSPTSTEEGSPAGSPTSTEEGSPAGSPTST |
| | EEGTSESATPESGPGTSTEPSEGSAPGAAEPEA |
| 17 | ASHHHHHHSPAGSPTSTEEGTSESATPESGPGTSTEPSEGSAPGTSESATPESGPGS |
| | EPATSGSETPGTSESATPESGPGSEPATSGSETPGTSESATPESGPGTSTEPSEGSAP |
| | GSPAGSPTSTEEGTSESATPESGPGSEPATSGSETPGTSESATPESGPGSPAGSPTST |
| | EEGSPAGSPTSTEEGTSTEPSEGSAPGTSESATPESGPGTSESATPESGPGTSESATP |
| | ESGPGSEPATSGSETPGSEPATSGSETPGSPAGSPTSTEEGTSTEPSEGSAPGTSTEP |
| | SEGSAPGGSAPGVRGLTGPATSGSETPGTDIQMTQSPSSLSASVGDRVTITCKASQ |
| | DVSIGVAWYQQKPGKAPKLLIYSASYRYTGVPSRFSGSGSGTDFTLTISSLQPEDF |
| | ATYYCQQYYTYPYTFGQGTKVEIKGATPPETGAETESPGETTGGSAESEPPGEGE |
| | VQLVESGGGLVQPGGSLRLSCAASGFTFTDYTMDWVRQAPGKGLEWVADVNPN |
| | SGGSIYNQRFKGRFTLSVDRSKNTLYLQMNSLRAEDTAVYYCARNLGPSFYFDY |
| | WGQGTLVTVSSGGGGSELVVTQEPSLTVSPGGTVTLTCRSSNGAVTSSNYANWV |
| | QQKPGQAPRGLIGGTNKRAPGTPARFSGSLLGGKAALTLSGVQPEDEAVYYCAL |
| | WYPNLWVFGGGTKLTVLGATPPETGAETESPGETTGGSAESEPPGEGEVQLLES |
| | GGGIVQPGGSLKLSCAASGFTFNTYAMNWVRQAPGKGLEWVARIRSKYNNYAT |
| | YYADSVKDRFTISRDDSKNTVYLQMNNLKTEDTAVYYCVRHENFGNSYVSWFAH |
| | WGQGTLVTVSSGTAEAASASGGVRGLTGPPGSPAGSPTSTEEGTSESATPESGPG |
| | TSTEPSEGSAPGSPAGSPTSTEEGTSTEPSEGSAPGTSTEPSEGSAPGTSESATPESG |
| | PGSEPATSGSETPGSEPATSGSETPGSPAGSPTSTEEGTSESATPESGPGTSTEPSEG |
| | SAPGTSTEPSEGSAPGSPAGSPTSTEEGTSTEPSEGSAPGTSTEPSEGSAPGTSESAT |
| | PESGPGTSTEPSEGSAPGTSESATPESGPGSEPATSGSETPGTSTEPSEGSAPGTSTE |
| | PSEGSAPGTSESATPESGPGTSESATPESGPGSPAGSPTSTEEGTSESATPESGPGSE |
| | PATSGSETPGTSESATPESGPGTSTEPSEGSAPGTSTEPSEGSAPGTSTEPSEGSAPG |
| | TSTEPSEGSAPGTSTEPSEGSAPGTSTEPSEGSAPGSPAGSPTSTEEGTSTEPSEGSA |
| | PGTSESATPESGPGSEPATSGSETPGTSESATPESGPGSEPATSGSETPGTSESATPE |
| | SGPGTSTEPSEGSAPGTSESATPESGPGSPAGSPTSTEEGSPAGSPTSTEEGSPAGSP |
| | TSTEEGTSESATPESGPGTSTEPSEGSAPGAAEPEA |
| 18 | SAGSPGSPAGSPTSTEEGTSESATPESGPGTSTEPSEGSAPGSPAGSPTSTEEGTSTE |
| | PSEGSAPGTSTEPSEGSAPGTSESATPESGPGSTPAESGSETPGSEPATSGSETPGSP |
| | AGSPTSTEEGTSESATPESGPGTSTEPSEGSAPGTSTEPSEGSAPGSPAGSPTSTEEG |
| | TSTEPSEGSAPGTSTEPSEGSAPGTSESATPESGPGTSTEPSEGSAPGTSESATPESG |
| | PGSEPATSGSTETPGTSTEPSEGSAPGTSTEPSEGSAPGTSESATPESGPGTSESATP |
| | ESGPGSPAGSPTSTEEGTSESATPESGPGSEPATSGSETPGTSESATPESGPGTSTEP |
| | SEGSAPGTSTEPSEGSAPGTSTEPSEGSAPGTSTEPSEGSAPGTSTEPSEGSAPGTST |
| | EPSEGSAPGSPAGSPTSTEEGTSTEPSEGSAPGTSESATPESGPGSEPATSGSETPGT |
| | SESATPESGPGSEPATSGSETPGTSESATPESGPGTSTEPSEGSAPGTSESATPESGP |
| | GSPAGSPTSTEEGSPAGSPTSTEEGSPAGSPTSTEEGTSESATPESGPGTSTEPSEGS |
| | APGTSESATPESGPGSEPATSGSETPGTSESATPESGPGSEPATSGSETPGTSESATP |
| | ESGPGTSTEPSEGSAPGSPAGSPTSTEEGTSESATPESGPGSEPATSGSETPGTSESA |
| | TPESGPGSPAGSPTSTEEGSPAGSPTSTEEGTSTEPSEGSAPGTSESATPESGPGTSE |
| | SATPESGPGTSESATPESGPGSEPATSGSETPGSEPATSGSETPGSPAGSPTSTEEGT |
| | STEPSEGSAPGTSTEPSEGSAPGSEPATSGSETPGTSESATPESGPGTSTEPSEGSAP |
| | GSPAGSPTSTEEGTSESATPESGPGTSTEPSEGSAPGSPAGSPTSTEEGTSTEPSEGS |
| | APGTSTEPSEGSAPGTSESATPESGPGSEPATSGSETPGSEPATSGSETPGSPAGSPT |
| | STEEGTSESATPESGPGTSTEPSEGSAPGTSTEPSEGSAPGSPAGSPTSTEEGTSTEP |
| | SEGSAPGTSTEPSEGSAPGTSESATPESGPGTSTEPSEGSAPGTSESATPESGPGSEP |
| | ATSGSETPGTSTEPSEGSAPGTSTEPSEGSAPGTSESATPESGPGTESASASGRAAN |
| | ETPPGLTGAATSGSETPGTEIVLTQSPATLSLSPGERATLSCKASQDVSIGVAWYQ |
| | QKPGQAPRLLIYSASYRYSGVPARFSGSGSGTDFTLTISSLEPEDFAVYYCQQYYIY |
| | PYTFGQGTKVEIKGATPPETGAETESPGETTGGSAESEPPGEGQVQLVQSGVEVK |
| | KPGASVKVSCKASGFTFTDYTMDWVRQAPGQGLEWMADVNPNSGGSIYNQRFK |
| | GRVTLTTDSSTTTAYMELKSLQFDDTAVYYCARNLGPSFYFDYWGQGTLVTVSS |
| | GGGSELVVTQEPSLTVSPGGTVTLTCRSSNGAVTSSNYANWVQQKPGQAPRGLIG |
| | GTNKRAPGTPARFSGSLLGGKAALTLSGVQPEDEAVYYCALWYPNLWVFGGGT |
| | KLTVLGATPPETGAETESPGETTGGSAESEPPGEGEVQLLESGGGIVQPGGSLKLS |
| | CAASGFTFNTYAMNWVRQAPGKGLEWVARIRSKYNNYATYYADSVKDRFTISRD |
| | DSKNTVYLQMNNLKTEDTAVYYCVRHENFGNSYVSWFAHWGQGTLVTVSSGTA |
| | EAASASGASGRAANETPPGLTGAGSETPGSPAGSPTSTEEGTSESATPESGPGTSTE |
| | PSEGSAPGTSTEPSEGSAPGSPAGSPTSTEEGTSTEPSEGSAPGTSTEPSEGSAPGTS |
| | ESATPESGPGTSTEPSEGSAPGTSESATPESGPGSEPATSGSETPGTSTEPSEGSAPG |
| | TSTEPSEGSAPGTSESATPESGPGTSESATPESGPGSPAGSPTSTEEGTSESATPESG |
| | PGSEPATSGSETPGTSESATPESGPGTSTEPSEGSAPGTSTEPSEGSAPGTSTEPSEG |
| | SAPGTSTEPSEGSAPGTSTEPSEGSAPGTSTEPSEGSAPGSPAGSPTSTEEGTSTEPS |
| | EGSAPGTSESATPESGPGSEPATSGSETPGTSESATPESGPGSEPATSGSETPGTSES |
| | ATPESGPGTSTEPSEGSAPGTSESATPESGPGSPAGSPTSTEEGSPAGSPTSTEEGSP |
| | AGSPTSTEEGTSESATPESGPGTSTEPSEGSAPGTSESATPESGPGSEPATSGSTETG |
| | TSESATPESGPGSEPATSGSETPGTSESATPESGPGTSTEPSEGSAPGSPAGSPTSTE |
| | EGTSESATPESGPGSEPATS |
| 19 | ASSPAGSPTSTESGTSESATPESGPGTETEPSEGSAPGTSESATPESGPGSEPATSGS |
| | ETPGTSESATPESGPGSTPAESGSETPGTSESATPESGPGTSTEPSEGSAPGSPAGSP |
| | TSTEEGTSESATPESGPGESPATSGSTPEGTSESATPESGPGSPAGSPTSTEEGSPAG |
| | SPTSTEEGTSTEPSEGSAPGTSESATPESGPGTSESATPESGPGTSESATPESGPGSE |
| | PATSGSETPGSEPATSGSETPGSPAGSPTSTEEGTSTEPSEGSAPGTSTEPSEGSAPG |
| | GSAPTTGRAGEAANATSAGATGPATSGSETPGTDIQMTQSPSSLSASVGDRVTITC |
| | RASQDVNTAVAWYQQKPGKAPKLLIYSASFLYSGVPSRFSGSRSGTDFTLTISSLQ |
| | PEDFATYYCQQHYTTPPTFGQGTKVEIKGATPPETGAETESPGETTGGSAESEPP |
| | GEGEVQLVESGGGLVQPGGSLRLSCAASGFNIKDTYIHWVRQAPGKGLEWVARI |
| | YPTNGYTRYADSVKGRFTISADTSKNTAYLQMNSLRAEDTAVYYCSRWGGDGFY |
| | AMDYWGQGTLVTVSSGGGSELVVTQEPSLTVSPGGTVTLTCRSSNGAVTSSNYA |
| | NWVQQKPGQAPRGLIGGTNKRAPGTPARFSGSLLGGKAALTLSGVQPEDEAVYY |
| | CALWYPNLWVFGGGTKLTVLGATPPETGAETESPGETTGGSAESEPPGEGEVQL |
| | LESGGGIVQPGGSLKLSCAASGFTFNTYAMNWVRQAPGKGLEWVARIRSKYNNY |
| | ATYYADSVKDRFTISRDDSKNTVYLQMNNLKTEDTAVYYCVRHENFGNSYVSWF |
| | AHWGQGTLVTVSSGTAEAASASGTTGRAGEAANATSAGATGPSAGSPGSPAGSPT |
| | STEEGTSESATPESGPGTSTEPSEGSAPGSPAGSPTSTEEGTSTEPSEGSAPGTSTEP |
| | SEGSAPGTSESATPESGPGSEPATSGSETPGSEPATSGSETPGSPAGSPTSTEEGTSE |
| | SATPESGPGTSTEPSEGSAPGTSTEPSEGSAPGSPAGSPTSTEEGTSTEPSEGSAPGT |
| | STEPSEGSAPGTSESATPESGPGTSTEPSEGSAPGTSESATPESGPGSEPATSGSETP |
| | GTSTEPSEGSAPGTSTEPSEGSAPGTSESATPESGPGTSESATPESGPGSPAGSPTST |
| | EEGTSESATPESGPGSEPATSGSETPGTSESATPESGPGTSTEPSEGSAPGTSTEPSE |
| | GSAPGTSTEPSEGSAPGTSTEPSEGSAPGTSTEPSEGSAPGTSTEPSEGSAPGSPAGS |
| | PTSTEEGTSTEPSEGSAPGTSESATPESGPGSEPATSGSETPGTSESATPESGPGSEP |
| | ATSGSETPGTSESATPESGPGTSTEPSEGSAPGTSESATPESGPGSPAGSPTSTEEGS |
| | PAGSPTSTEEGSPAGSPTSTEEGTSESATPESGPGTSTEPSEGSAPGTSESATPESGP |
| | GSEPATSGSETPGTSESATPESGPGSEPATSGSETPGTSESATPESGPGTSTEPSEGS |
| | APGSPAGSPTSTEEGTSESATPESGPGSEPATSGSETPGTSESATPESGPGSPAGSPT |
| | STEEGSPAGSPTSTEEGTSTEPSEGSAPGTSESATPESGPGTSESATPESGPGTSESA |
| | TPESGPGSEPATSGSETPGSEPATSGSETPGSPAGSPTSTEEGTSTEPSEGSAPGTST |
| | EPSEGSAPGSEPATSGSETPGTSESATPESGPGTSTEPSEGSAPGSPAGSPTSTEEGT |
| | SESATPESGPGTSTEPSEGSAPGSPAGSPTSTEEGTSTEPSEGSAPGTSTEPSEGSAP |
| | GTSESATPESGPGSEPATSGSETPGSEPATSGSTETPGSPAGSPTSTEEGTSESATPE |
| | SGPGTSTEPSEGSAPGTSTEPSEGSAPGSPAGSPTSTEEGTSTEPSEGSAPGTATESP |
| | EGSAPGTSESATPESGPGTSTEPSEGSAPGTSAESATPESGPGSEPATSGSETPGTST |
| | EPSEGSAPGTSTEPSEGSAPGTSESATPESGPGTESAS |
| 20 | ASHHHHHHSPAGSPTSTEEGTSESATPESGPGTSTEPSEGSAPGTSESATPESGPGS |
| | EPATSGSETPGTSESATPESGPGSEPATSGSETPGTSESATPESGPGTSTEPSEGSAP |
| | GSPAGSPTSTEEGTSESATPESGPGSEPATSGSETPGTSESATPESGPGSPAGSPTST |
| | EEGSPAGSPTSTEEGTSTEPSEGSAPGTSESATPESGPGTSESATPESGPGTSESATP |
| | ESGPGSEPATSGSETPGSEPATSGSETPGSPAGSPTSTEEGTSTEPSEGSAPGTSTEP |
| | SEGSAPGGSAPGPGGVAAAATSGSETPGTDIQMTQSPSSLSASVGDRVTITCKASQ |
| | DVSIGVAWYQQKPGKAPKLLIYSASYRYTGVPSRFSGSGSGTDFTLTISSLQPEDF |
| | ATYYCQQYYTYPYTFGQGTKVEIKGATPPETGAETESPGETTGGSAESEPPGEGE |
| | VQLVESGGGLVQPGGSLRLSCAASGFTFTDYTMDWVRQAPGKGLEWVADVNPN |
| | SGGSIYNQRFKGRFTLSVDRSKNTLYLQMNSLRAEDTAVYYCARNLGPSFYFDY |
| | WGQGTLVTVSSGGGGSELVVTQEPSLTVSPGGTVTLTCRSSNGAVTSSNYANWV |
| | QQKPGQAPRGLIGGTNKRAPGTPARFSGSLLGGKAALTLSGVQPEDEAVYYCAL |
| | WYPNLWVFGGGTKLTVLGATPPETGAETESPGETTGGSAESEPPGEGEVQLLES |
| | GGGIVQPGGSLKLSCAASGFTFNTYAMNWVRQAPGKGLEWVARIRSKYNNYAT |
| | YYADSVKDRFTISRDDSKNTVYLQMNNLKTEDTAVYYCVRHENFGNSYVSWFAH |
| | WGQGTLVTVSSGTAEAASASGGPGGVAAAPGSPAGSPTSTEEGTSESATPESGPG |
| | TSTEPSEGSAPGSPAGSPTSTEEGTSTEPSEGSAPGTSTEPSEGSAPGTSESATPESG |
| | PGSEPATSGSETPGSEPATSGSETPGSPAGSPTSTEEGTSESATPESGPGTSTEPSEG |
| | SAPGTSTEPSEGSAPGSPAGSPTSTEEGTSTEPSEGSAPGTSTEPSEGSAPGTSESAT |
| | PESGPGTSTEPSEGSAPGTSESATPESGPGSEPATSGSETPGTSTEPSEGSAPGTSTE |
| | PSEGSAPGTSESATPESGPGTSESATPESGPGSPAGSPTSTEEGTSESATPESGPGSE |
| | PATSGSETPGTSESATPESGPGTSTEPSEGSAPGTSTEPSEGSAPGTSTEPSEGSAPG |
| | TSTEPSEGSAPGTSTEPSEGSAPGTSTEPSEGSAPGSPAGSPTSTEEGTSTEPSEGSA |
| | PGTSESATPESGPGSEPATSGSETPGTSESATPESGPGSEPATSGSETPGTSESATPE |
| | SGPGTSTEPSEGSAPGTSESATPESGPGSPAGSPTSTEEGSPAGSPTSTEEGSPAGSP |
| | TSTEEGTSESATPESGPGTSTEPSEGSAPGAAEPEA |
| 21 | ASHHHHHHSPAGSPTSTEEGTSESATPESGPGTSTEPSEGSAPGTSESATPESGPGS |
| | EPATSGSETPGTSESATPESGPGSEPATSGSETPGTSESATPESGPGTSTEPSEGSAP |
| | GSPAGSPTSTEEGTSESATPESGPGSEPATSGSETPGTSESATPESGPGSPAGSPTST |
| | EEGSPAGSPTSTEEGTSTEPSEGSAPGTSESATPESGPGTSESATPESGPGTSESATP |
| | ESGPGSEPATSGSETPGSEPATSGSETPGSPAGSPTSTEEGTSTEPSEGSAPGTSTEP |
| | SEGSAPGGSAPVSKRFPVGATSGSETPGTDIQMTQSPSSLSASVGDRVTITCKASQ |
| | DVSIGVAWYQQKPGKAPKLLIYSASYRYTGVPSRFSGSGSGTDFTLTISSLQPEDF |
| | ATYYCQQYYTYPYTFGQGTKVEIKGATPPETGAETESPGETTGGSAESEPPGEGE |
| | VQLVESGGGLVQPGGSLRLSCAASGFTFTDYTMDWVRQAPGKGLEWVADVNPN |
| | SGGSIYNQRFKGRFTLSVDRSKNTLYLQMNSLRAEDTAVYYCARNLGPSFYFDY |
| | WGQGTLVTVSSGGGGSELVVTQEPSLTVSPGGTVTLTCRSSNGAVTSSNYANWV |
| | QQKPGQAPRGLIGGTNKRAPGTPARFSGSLLGGKAALTLSGVQPEDEAVYYCAL |
| | WYPNLWVFGGGTKLTVLGATPPETGAETESPGETTGGSAESEPPGEGEVQLLES |
| | GGGIVQPGGSLKLSCAASGFTFNTYAMNWVRQAPGKGLEWVARIRSKYNNYAT |
| | YYADSVKDRFTISRDDSKNTVYLQMNNLKTEDTAVYYCVRHENFGNSYVSWFAH |
| | WGQGTLVTVSSGTAEAASASGVSKRFPVGPGSPAGSPTSTEEGTSESATPESGPGT |
| | STEPSEGSAPGSPAGSPTSTEEGTSTEPSEGSAPGTSTEPSEGSAPGTSESATPESGP |
| | GSEPATSGSETPGSEPATSGSETPGSPAGSPTSTEEGTSESATPESGPGTSTEPSEGS |
| | APGTSTEPSEGSAPGSPAGSPTSTEEGTSTEPSEGSAPGTSTEPSEGSAPGTSESATP |
| | ESGPGTSTEPSEGSAPGTSESATPESGPGSEPATSGSETPGTSTEPSEGSAPGTSTEP |
| | SEGSAPGTSESATPESGPGTSESATPESGPGSPAGSPTSTEEGTSESATPESGPGSEP |
| | ATSGSETPGTSESATPESGPGTSTEPSEGSAPGTSTEPSEGSAPGTSTEPSEGSAPGT |
| | STEPSEGSAPGTSTEPSEGSAPGTSTEPSEGSAPGSPAGSPTSTEEGTSTEPSEGSAP |
| | GTSESATPESGPGSEPATSGSETPGTSESATPESGPGSEPATSGSETPGTSESATPES |
| | GPGTSTEPSEGSAPGTSESATPESGPGSPAGSPTSTEEGSPAGSPTSTEEGSPAGSPT |
| | STEEGTSESATPESGPGTSTEPSEGSAPGAAEPEA |
| 22 | ASHHHHHHSPAGSPTSTEEGTSESATPESGPGTSTEPSEGSAPGTSESATPESGPGS |
| | EPATSGSETPGTSESATPESGPGSEPATSGSETPGTSESATPESGPGTSTEPSEGSAP |
| | GSPAGSPTSTEEGTSESATPESGPGSEPATSGSETPGTSESATPESGPGSPAGSPTST |
| | EEGSPAGSPTSTEEGTSTEPSEGSAPGTSESATPESGPGTSESATPESGPGTSESATP |
| | ESGPGSEPATSGSETPGSEPATSGSETPGSPAGSPTSTEEGTSTEPSEGSAPGTSTEP |
| | SEGSAPGGSAPGPGGVAAAATSGSETPGTDIQMTQSPSSLSASVGDRVTITCQASQ |
| | DISNYLNWYQQKPGKAPKLLIYDASNLETGVPSRFSGSGSGTDFTFTISSLQPEDIA |
| | TYFCQHFDHLPLAFGGGTKVEIKGATPPETGAETESPGETTGGSAESEPPGEGQV |
| | QLQESGPGLVKPSETLSLTCTVSGGSVSSGDYYWTWIRQSPGKGLEWIGHIYYSG |
| | NTNYNPSLKSRLTISIDTSKTQFSLKLSSVTAADTAIYYCVRDRVTGAFDIWGQGT |
| | MVTVSSGGGGSELVVTQEPSLTVSPGGTVTLTCRSSTGAVTTSNYANWVQQKPG |
| | QAPRGLIGGTNKRAPGTPARFSGSLLGGKAALTLSGVQPEDEAEYYCALWYSNL |
| | WVFGGGTKLTVLGATPPETGAETESPGETTGGSAESEPPGEGEVQLLESGGGLV |
| | QPGGSLKLSCAASGFTFNTYAMNWVRQAPGKGLEWVARIRSKYNNYATYYADS |
| | VKDRFTISRDDSKNTAYLQMNNLKTEDTAVYYCVRHGNFGNSYVSWFAYWGQG |
| | TLVTVSSGTAEAASASGGPGGVAAAPGSPAGSPTSTEEGTSESATPESGPGTSTEPS |
| | EGSAPGSPAGSPTSTEEGTSTEPSEGSAPGTSTEPSEGSAPGTSESATPESGPGSEPA |
| | TSGSETPGSEPATSGSETPGSPAGSPTSTEEGTSESATPESGPGTSTEPSEGSAPGTS |
| | TEPSEGSAPGSPAGSPTSTEEGTSTEPSEGSAPGTSTEPSEGSAPGTSESATPESGPG |
| | TSTEPSEGSAPGTSESATPESGPGSEPATSGSETPGTSTEPSEGSAPGTSTEPSEGSA |
| | PGTSESATPESGPGTSESATPESGPGSPAGSPTSTEEGTSESATPESGPGSEPATSGS |
| | ETPGTSESATPESGPGTSTEPSEGSAPGTSTEPSEGSAPGTSTEPSEGSAPGTSTEPS |
| | EGSAPGTSTEPSEGSAPGTSTEPSEGSAPGSPAGSPTSTEEGTSTEPSEGSAPGTSES |
| | ATPESGPGSEPATSGSETPGTSESATPESGPGSEPATSGSETPGTSESATPESGPGTS |
| | TEPSEGSAPGTSESATPESGPGSPAGSPTSTEEGSPAGSPTSTEEGSPAGSPTSTEEG |
| | TSESATPESGPGTSTEPSEGSAPGAAEPEA |
| 23 | ASSPAGSPTSTESGTSESATPESGPGTSTEPSEGSAPGTSESATPESGPGSEPATSGS |
| | ETPGTSESATPESGPGSTPAESGSETPGTSESATPESGPGTSTEPSEGSAPGSPAGSP |
| | TSTEEGTSESATPESGPGSEPATSGSETPGTSESATPESGPGSPAGSPTSTEEGSPAG |
| | SPTSTEEGTSTEPSEGSAPGTSESATPESGPGTSESATPESGPGTSESATPESGPGSE |
| | PATSGSETPGSEPATSGSETPGSPAGSPTSTEEGTSTEPSEGSAPGTSTEPSEGSAPG |
| | GSAPASGRSTNAGPPGLTGPATSGSETPGTDIQMTQSPSSLSASVGDRVTITCRAS |
| | QDVNTAVAWYQQKPGKAPKLLIYSASFLYSGVPSRFSGSRSGTDFTLTISSLQPED |
| | FATYYCQQHYTTPPTFGQGTKVEIKGATPPETGAETESPGETTGGSAESEPPGEG |
| | EVQLVESGGGLVQPGGSLRLSCAASGFNIKDTYIHWVRQAPGKGLEWVARIYPT |
| | NGYTRYADSVKGRFTISADTSKNTAYLQMNSLRAEDTAVYYCSRWGGDGFYAM |
| | DYWGQGTLVTVSSGGGSELVVTQEPSLTVSPGGTVTLTCRSSTGAVTTSNYANW |
| | VQQKPGQAPRGLIGGTNKRAPGTPARFSGSLLGGKAALTLSGVQPEDEAEYYCA |
| | LWYSNLWVFGGGTKLTVLGATPPETGAETESPGETTGGSAESEPPGEGEVQLLE |
| | SGGGLVQPGGSLKLSCAASGFTFNTYAMNWVRQAPGKGLEWVARIRSKYNNYA |
| | TYYADSVKDRFTISRDDSKNTAYLQMNNLKTEDTAVYYCVRHGNFGNSYVSWFA |
| | YWGQGTLVTVSSGTAEAASASGASGRSTNAGPPGLTGPPGSPAGSPTSTEEGTSES |
| | ATPESGPGSEPATSGSETPGTSESATPESGPGTSTEPSEGSAPGTSTEPSEGSAPGTS |
| | TEPSEGSAPGTSTEPSEGSAPGTSTEPSEGSAPGTSTEPSEGSAPGSPAGSPTSTEEG |
| | TSTEPSEGSAPGTSESATPESGPGSEPATSGSETPGTSESATPESGPGSEPATSGSET |
| | PGTSESATPESGPGTSTEPSEGSAPGTSESATPESGPGSPAGSPTSTEEGSPAGSPTS |
| | TEEGSPAGSPTSTEEGTSESATPESGPGTSTEPSEGSAPGTSESATPESGPGSEPATS |
| | GSETPGTSESATPESGPGSEPATSGSETPGTSESATPESGPGTSTEPSEGSAPGSPAG |
| | SPTSTEEGTSESATPESGPGSEPATSGSETPGTSESATPESGPGSPAGSPTSTEEGSP |
| | AGSPTSTEEGTSTEPSEGSAPGTSESATPESGPGTSESATPESGPGTSESATPESGPG |
| | SEPATSGSETPGSEPATSGSETPGSPAGSPTSTEEGTSTEPSEGSAPGTESTPSEGSA |
| | PGSEPATSGSETPGTSESATPESGPGTSTEPSEGSAPG |
| 24 | ASHHHHHHSPAGSPTSTEEGTSESATPESGPGTSTEPSEGSAPGTSESATPESGPGS |
| | EPATSGSETPGTSESATPESGPGSEPATSGSETPGTSESATPESGPGTSTEPSEGSAP |
| | GSPAGSPTSTEEGTSESATPESGPGSEPATSGSETPGTSESATPESGPGSPAGSPTST |
| | EEGSPAGSPTSTEEGTSTEPSEGSAPGTSESATPESGPGTSESATPESGPGTSESATP |
| | ESGPGSEPATSGSETPGSEPATSGSETPGSPAGSPTSTEEGTSTEPSEGSAPGTSTEP |
| | SEGSAPGGSAPQPAHLTFPATSGSETPGTDIQMTQSPSSLSASVGDRVTITCKASQD |
| | VSIGVAWYQQKPGKAPKLLIYSASYRYTGVPSRFSGSGSGTDFTLTISSLQPEDFA |
| | TYYCQQYYIYPYTFGQGTKVEIKGATPPETGAETESPGETTGGSAESEPPGEGEV |
| | QLVESGGGLVQPGGSLRLSCAASGFTFTDYTMDWVRQAPGKGLEWVADVNPNS |
| | GGSIYNQRFKGRFTLSVDRSKNTLYLQMNSLRAEDTAVYYCARNLGPSFYFDYW |
| | GQGTLVTVSSGGGGSELVVTQEPSLTVSPGGTVTLTCRSSNGAVTSSNYANWVQ |
| | QKPGQAPRGLIGGTNKRAPGTPARFSGSLLGGKAALTLSGVQPEDEAVYYCALW |
| | YPNLWVFGGGTKLTVLGATPPETGAETESPGETTGGSAESEPPGEGEVQLLESGG |
| | GIVQPGGSLKLSCAASGFTFNTYAMNWVRQAPGKGLEWVARIRSKYNNYATYY |
| | ADSVKDRFTISRDDSKNTVYLQMNNLKTEDTAVYYCVRHENFGNSYVSWFAHW |
| | GQGTLVTVSSGTAEAASASGQPAHLTFPPGSPAGSPTSTEEGTSESATPESGPGTST |
| | EPSEGSAPGSPAGSPTSTEEGTSTEPSEGSAPGTSTEPSEGSAPGTSESATPESGPGS |
| | EPATSGSETPGSEPATSGSETPGSPAGSPTSTEEGTSESATPESGPGTSTEPSEGSAP |
| | GTSTEPSEGSAPGSPAGSPTSTEEGTSTEPSEGSAPGTSTEPSEGSAPGTSESATPES |
| | GPGTSTEPSEGSAPGTSESATPESGPGSEPATSGSETPGTSTEPSEGSAPGTSTEPSE |
| | GSAPGTSESATPESGPGTSESATPESGPGSPAGSPTSTEEGTSESATPESGPGSEPAT |
| | SGSETPGTSESATPESGPGTSTEPSEGSAPGTSTEPSEGSAPGTSTEPSEGSAPGTST |
| | EPSEGSAPGTSTEPSEGSAPGTSTEPSEGSAPGSPAGSPTSTEEGTSTEPSEGSAPGT |
| | SESATPESGPGSEPATSGSETPGTSESATPESGPGSEPATSGSETPGTSESATPESGP |
| | GTSTEPSEGSAPGTSESATPESGPGSPAGSPTSTEEGSPAGSPTSTEEGSPAGSPTST |
| | EEGTSESATPESGPGTSTEPSEGSAPGAAEPEA |
| 25 | ASSPAGSPTSTESGTSESATPESGPGTSTEPSEGSAPGTSESATPESGPGSEPATSGS |
| | ETPGTSESATPESGPGSTPAESGSETPGTSESATPESGPGTSTEPSEGSAPGSPAGSP |
| | TSTEEGTSESATPESGPGEEPATSGSTPEGTSESATPESGPGSPAGSPTSTEEGSPAG |
| | SPTSTEEGTSTEPSEGSAPGTSESATPESGPGTSESATPESGPGTSESATPESGPGSE |
| | PATSGSETPGSEPATSGSETPGSPAGSPTSTEEGTSTEPSEGSAPGTSTEPSEGSAPG |
| | GSAPEAGRSANHTPAGLTGPATSGSETPGTDIQMTQSPSSLSASVGDRVTITCRAS |
| | QDVNTAVAWYQQKPGKAPKLLIYSASFLYSGVPSRFSGSRSGTDFTLTISSLQPED |
| | FATYYCQQHYTTPPTFGQGTKVEIKGATPPETGAETESPGETTGGSAESEPPGEG |
| | EVQLVESGGGLVQPGGSLRLSCAASGFNIKDTYIHWVRQAPGKGLEWVARIYPT |
| | NGYTRYADSVKGRFTISADTSKNTAYLQMNSLRAEDTAVYYCSRWGGDGFYAM |
| | DYWGQGTLVTVSSGGGSELVVTQEPSLTVSPGGTVTLTCRSSNGAVTSSNYANW |
| | VQQKPGQAPRGLIGGTNKRAPGTPARFSGSLLGGKAALTLSGVQPEDEAVYYCA |
| | LWYPNLWVFGGGTKLTVLGATPPETGAETESPGETTGGSAESEPPGEGEVQLLE |
| | SGGGIVQPGGSLKLSCAASGFTFNTYAMNWVRQAPGKGLEWVARIRSKYNNYAT |
| | YYADSVKDRFTISRDDSKNTVYLQMNNLKTEDTAVYYCVRHENFGNSYVSWFAH |
| | WGQGTLVTVSSGTAEAASASGEAGRSANHTPAGLTGPSAGSPGSPAGSPTSTEEG |
| | TSESATPESGPGTSTEPSEGSAPGSPAGSPTSTEEGTSTEPSEGSAPGTSTEPSEGSA |
| | PGTSESATPESGPGSEPATSGSETPGSEPATSGSETPGSPAGSPTSTEEGTSESATPE |
| | SGPGTSTEPSEGSAPGTSTEPSEGSAPGSPAGSPTSTEEGTSTEPSEGSAPGTSTEPS |
| | EGSAPGTSESATPESGPGTSTEPSEGSAPGTSESATPESGPGSEPATSGSETPGTSTE |
| | PSEGSAPGTSTEPSEGSAPGTSESATPESGPGTSESATPESGPGSPAGSPTSTEEGTS |
| | ESATPESGPGSEPATSGSETPGTSESATPESGPGTSTEPSEGSAPGTSTEPSEGSAPG |
| | TSTEPSEGSAPGTSTEPSEGSAPGTSTEPSEGSAPGTSTEPSEGSAPGSPAGSPTSTE |
| | EGTSTEPSEGSAPGTSESATPESGPGSEPATSGSETPGTSESATPESGPGSEPATSGS |
| | ETPGTSESATPESGPGTSTEPSEGSAPGTSESATPESGPGSPAGSPTSTEEGSPAGSP |
| | TSTEEGSPAGSPTSTEEGTSESATPESGPGTSTEPSEGSAPGTSESATPESGPGSEPA |
| | TSGSETPGTSESATPESGPGSEPATSGSETPGTSESATPESGPGTSTEPSEGSAPGSP |
| | AGSPTSTEEGTSESATPESGPGSEPATSGSETPGTSESATPESGPGSPAGSPTSTEEG |
| | SPAGSPTSTEEGTSTEPSEGSAPGTSESATPESGPGTSESATPESGPGTSESATPESG |
| | PGSEPATSGSETPGSEPATSGSETPGSPAGSPTSTEEGTSTEPSEGSAPGTSTEPSEG |
| | SAPGSEPATSGSETPGTSESATPESGPGTSTEPSEGSAPGSPAGSPTSTEEGTSESAT |
| | PESGPGTSTEPSEGSAPGSPAGSPTSTEEGTSTEPSEGSAPGTSTEPSEGSAPGTSES |
| | ATPESGPGSEPATSGSETPGSEPATSGSTETPGSPAGSPTSTEEGTSESATPESGPGT |
| | STEPSEGSAPGTSTEPSEGSAPGSPAGSPTSTEEGTSTEPSEGSAPGTATESPEGSAP |
| | GTSESATPESGPGTSTEPSEGSAPGTSAESATPESGPGSEPATSGSETPGTSTEPSEG |
| | SAPGTSTEPSEGSAPGTSESATPESGPGTESAS |
| 26 | ASHHHHHHSPAGSPTSTEEGTSESATPESGPGTSTEPSEGSAPGTSESATPESGPGS |
| | EPATSGSETPGTSESATPESGPGSEPATSGSETPGTSESATPESGPGTSTEPSEGSAP |
| | GSPAGSPTSTEEGTSESATPESGPGSEPATSGSETPGTSESATPESGPGSPAGSPTST |
| | EEGSPAGSPTSTEEGTSTEPSEGSAPGTSESATPESGPGTSESATPESGPGTSESATP |
| | ESGPGSEPATSGSETPGSEPATSGSETPGSPAGSPTSTEEGTSTEPSEGSAPGTSTEP |
| | SEGSAPGGSAPEAGSPGKDGVRGLTGPATSGSETPGTDIQMTQSPSSLSASVGDRV |
| | TITCKASQDVSIGVAWYQQKPGKAPKLLIYSASYRYTGVPSRFSGSGSGTDFTLTI |
| | SSLQPEDFATYYCQQYYTYPYTFGQGTKVEIKGATPPETGAETESPGETTGGSAES |
| | EPPGEGEVQLVESGGGLVQPGGSLRLSCAASGFTFTDYTMDWVRQAPGKGLEW |
| | VADVNPNSGGSIYNQRFKGRFTLSVDRSKNTLYLQMNSLRAEDTAVYYCARNLG |
| | PSFYFDYWGQGTLVTVSSGGGGSELVVTQEPSLTVSPGGTVTLTCRSSNGAVTSS |
| | NYANWVQQKPGQAPRGLIGGTNKRAPGTPARFSGSLLGGKAALTLSGVQPEDEA |
| | VYYCALWYPNLWVFGGGTKLTVLGATPPETGAETESPGETTGGSAESEPPGEGE |
| | VQLLESGGGIVQPGGSLKLSCAASGFTFNTYAMNWVRQAPGKGLEWVARIRSKY |
| | NNYATYYADSVKDRFTISRDDSKNTVYLQMNNLKTEDTAVYYCVRHENFGNSYV |
| | SWFAHWGQGTLVTVSSGTAEAASASGEAGSPGKDGVRGLTGPPGSPAGSPTSTE |
| | EGTSESATPESGPGTSTEPSEGSAPGSPAGSPTSTEEGTSTEPSEGSAPGTSTEPSEG |
| | SAPGTSESATPESGPGSEPATSGSETPGSEPATSGSETPGSPAGSPTSTEEGTSESAT |
| | PESGPGTSTEPSEGSAPGTSTEPSEGSAPGSPAGSPTSTEEGTSTEPSEGSAPGTSTE |
| | PSEGSAPGTSESATPESGPGTSTEPSEGSAPGTSESATPESGPGSEPATSGSETPGTS |
| | TEPSEGSAPGTSTEPSEGSAPGTSESATPESGPGTSESATPESGPGSPAGSPTSTEEG |
| | TSESATPESGPGSEPATSGSETPGTSESATPESGPGTSTEPSEGSAPGTSTEPSEGSA |
| | PGTSTEPSEGSAPGTSTEPSEGSAPGTSTEPSEGSAPGTSTEPSEGSAPGSPAGSPTS |
| | TEEGTSTEPSEGSAPGTSESATPESGPGSEPATSGSETPGTSESATPESGPGSEPATS |
| | GSETPGTSESATPESGPGTSTEPSEGSAPGTSESATPESGPGSPAGSPTSTEEGSPAG |
| | SPTSTEEGSPAGSPTSTEEGTSESATPESGPGTSTEPSEGSAPGAAEPEA |
| 27 | ASSPAGSPTSTESGTSESATPESGPGTSTEPSEGSAPGTSESATPESGPGSEPATSGS |
| | ETPGTSESATPESGPGSTPAESGSETPGTSESATPESGPGTSTEPSEGSAPGSPAGSP |
| | TSTEEGTSESATPESGPGSEPATSGSETPGTSESATPESGPGSPAGSPTSTEEGSPAG |
| | SPTSTEEGTSTEPSEGSAPGTSESATPESGPGTSESATPESGPGTSESATPESGPGSE |
| | PATSGSETPGSEPATSGSETPGSPAGSPTSTEEGTSTEPSEGSAPGTSTEPSEGSAPG |
| | GSAPRTGRTGESANETPAGLGGPATSGSETPGTEIVLTQSPATLSLSPGERATLSC |
| | KASQDVSIGVAWYQQKPGQAPRLLIYSASYRYSGVPARFSGSGSGTDFTLTISSLE |
| | PEDFAVYYCQQYYIYPYTFGQGTKVEIKGATPPETGAETESPGETTGGSAESEPPG |
| | EGQVQLVQSGVEVKKPGASVKVSCKASGFTFTDYTMDWVRQAPGQGLEWMAD |
| | VNPNSGGSIYNQRFKGRVTLTTDSSTTTAYMELKSLQFDDTAVYYCARNLGPSFY |
| | FDYWGQGTLVTVSSGGGSELVVTQEPSLTVSPGGTVTLTCRSSNGAVTSSNYAN |
| | WVQQKPGQAPRGLIGGTNKRAPGTPARFSGSLLGGKAALTLSGVQPEDEAVYYC |
| | ALWYPNLWVFGGGTKLTVLGATPPETGAETESPGETTGGSAESEPPGEGEVQLL |
| | ESGGGIVQPGGSLKLSCAASGFTFNTYAMNWVRQAPGKGLEWVARIRSKYNNYA |
| | TYYADSVKDRFTISRDDSKNTVYLQMNNLKTEDTAVYYCVRHENFGNSYVSWFA |
| | HWGQGTLVTVSSGTAEAASASGRTGRTGESANETPAGLGGPGSETPGSPAGSPTS |
| | TEEGTSESATPESGPGTSTEPSEGSAPGTSTEPSEGSAPGSPAGSPTSTEEGTSTEPS |
| | EGSAPGTSTEPSEGSAPGTSESATPESGPGTSTEPSEGSAPGTSESATPESGPGSEPA |
| | TSGSETPGTSTEPSEGSAPGTSTEPSEGSAPGTSESATPESGPGTSESATPESGPGSP |
| | AGSPTSTEEGTSESATPESGPGSEPATSGSETPGTSESATPESGPGTSTEPSEGSAPG |
| | TSTEPSEGSAPGTSTEPSEGSAPGTSTEPSEGSAPGTSTEPSEGSAPGTSTEPSEGSA |
| | PGSPAGSPTSTEEGTSTEPSEGSAPGTSESATPESGPGSEPATSGSETPGTSESATPE |
| | SGPGSEPATSGSETPGTSESATPESGPGTSTEPSEGSAPGTSESATPESGPGSPAGSP |
| | TSTEEGSPAGSPTSTEEGSPAGSPTSTEEGTSESATPESGPGTSTEPSEGSAPGTSES |
| | ATPESGPGSEPATSGSTETGTSESATPESGPGSEPATSGSETPGTSESATPESGPGTS |
| | TEPSEGSAPGSPAGSPTSTEEGTSESATPESGPGSEPATS |
| 28 | ASHHHHHHSPAGSPTSTEEGTSESATPESGPGTSTEPSEGSAPGTSESATPESGPGS |
| | EPATSGSETPGTSESATPESGPGSEPATSGSETPGTSESATPESGPGTSTEPSEGSAP |
| | GSPAGSPTSTEEGTSESATPESGPGSEPATSGSETPGTSESATPESGPGSPAGSPTST |
| | EEGSPAGSPTSTEEGTSTEPSEGSAPGTSESATPESGPGTSESATPESGPGTSESATP |
| | ESGPGSEPATSGSETPGSEPATSGSETPGSPAGSPTSTEEGTSTEPSEGSAPGTSTEP |
| | SEGSAPGGSAPSPEAQAAAATSGSETPGTDIQMTQSPSSLSASVGDRVTITCKASQ |
| | DVSIGVAWYQQKPGKAPKLLIYSASYRYTGVPSRFSGSGSGTDFTLTISSLQPEDF |
| | ATYYCQQYYTYPYTFGQGTKVEIKGATPPETGAETESPGETTGGSAESEPPGEGE |
| | VQLVESGGGLVQPGGSLRLSCAASGFTFTDYTMDWVRQAPGKGLEWVADVNPN |
| | SGGSIYNQRFKGRFTLSVDRSKNTLYLQMNSLRAEDTAVYYCARNLGPSFYFDY |
| | WGQGTLVTVSSGGGGSELVVTQEPSLTVSPGGTVTLTCRSSNGAVTSSNYANWV |
| | QQKPGQAPRGLIGGTNKRAPGTPARFSGSLLGGKAALTLSGVQPEDEAVYYCAL |
| | WYPNLWVFGGGTKLTVLGATPPETGAETESPGETTGGSAESEPPGEGEVQLLES |
| | GGGIVQPGGSLKLSCAASGFTFNTYAMNWVRQAPGKGLEWVARIRSKYNNYAT |
| | YYADSVKDRFTISRDDSKNTVYLQMNNLKTEDTAVYYCVRHENFGNSYVSWFAH |
| | WGQGTLVTVSSGTAEAASASGSPEAQAAAPGSPAGSPTSTEEGTSESATPESGPGT |
| | STEPSEGSAPGSPAGSPTSTEEGTSTEPSEGSAPGTSTEPSEGSAPGTSESATPESGP |
| | GSEPATSGSETPGSEPATSGSETPGSPAGSPTSTEEGTSESATPESGPGTSTEPSEGS |
| | APGTSTEPSEGSAPGSPAGSPTSTEEGTSTEPSEGSAPGTSTEPSEGSAPGTSESATP |
| | ESGPGTSTEPSEGSAPGTSESATPESGPGSEPATSGSETPGTSTEPSEGSAPGTSTEP |
| | SEGSAPGTSESATPESGPGTSESATPESGPGSPAGSPTSTEEGTSESATPESGPGSEP |
| | ATSGSETPGTSESATPESGPGTSTEPSEGSAPGTSTEPSEGSAPGTSTEPSEGSAPGT |
| | STEPSEGSAPGTSTEPSEGSAPGTSTEPSEGSAPGSPAGSPTSTEEGTSTEPSEGSAP |
| | GTSESATPESGPGSEPATSGSETPGTSESATPESGPGSEPATSGSETPGTSESATPES |
| | GPGTSTEPSEGSAPGTSESATPESGPGSPAGSPTSTEEGSPAGSPTSTEEGSPAGSPT |
| | STEEGTSESATPESGPGTSTEPSEGSAPGAAEPEA |
| 29 | ASHHHHHHSPAGSPTSTEEGTSESATPESGPGTSTEPSEGSAPGTSESATPESGPGS |
| | EPATSGSETPGTSESATPESGPGSEPATSGSETPGTSESATPESGPGTSTEPSEGSAP |
| | GSPAGSPTSTEEGTSESATPESGPGSEPATSGSETPGTSESATPESGPGSPAGSPTST |
| | EEGSPAGSPTSTEEGTSTEPSEGSAPGTSESATPESGPGTSESATPESGPGTSESATP |
| | ESGPGSEPATSGSETPGSEPATSGSETPGSPAGSPTSTEEGTSTEPSEGSAPGTSTEP |
| | SEGSAPGGSAPLTSDLQAQATSGSETPGTDIQMTQSPSSLSASVGDRVTITCQASQ |
| | DISNYLNWYQQKPGKAPKLLIYDASNLETGVPSRFSGSGSGTDFTFTISSLQPEDIA |
| | TYFCQHFDHLPLAFGGGTKVEIKGATPPETGAETESPGETTGGSAESEPPGEGQV |
| | QLQESGPGLVKPSETLSLTCTVSGGSVSSGDYYWTWIRQSPGKGLEWIGHIYYSG |
| | NTNYNPSLKSRLTISIDTSKTQFSLKLSSVTAADTAIYYCVRDRVTGAFDIWGQGT |
| | MVTVSSGGGGSELVVTQEPSLTVSPGGTVTLTCRSSTGAVTTSNYANWVQQKPG |
| | QAPRGLIGGTNKRAPGTPARFSGSLLGGKAALTLSGVQPEDEAEYYCALWYSNL |
| | WVFGGGTKLTVLGATPPETGAETESPGETTGGSAESEPPGEGEVQLLESGGGLV |
| | QPGGSLKLSCAASGFTFNTYAMNWVRQAPGKGLEWVARIRSKYNNYATYYADS |
| | VKDRFTISRDDSKNTAYLQMNNLKTEDTAVYYCVRHGNFGNSYVSWFAYWGQG |
| | TLVTVSSGTAEAASASGLTSDLQAQPGSPAGSPTSTEEGTSESATPESGPGTSTEPS |
| | EGSAPGSPAGSPTSTEEGTSTEPSEGSAPGTSTEPSEGSAPGTSESATPESGPGSEPA |
| | TSGSETPGSEPATSGSETPGSPAGSPTSTEEGTSESATPESGPGTSTEPSEGSAPGTS |
| | TEPSEGSAPGSPAGSPTSTEEGTSTEPSEGSAPGTSTEPSEGSAPGTSESATPESGPG |
| | TSTEPSEGSAPGTSESATPESGPGSEPATSGSETPGTSTEPSEGSAPGTSTEPSEGSA |
| | PGTSESATPESGPGTSESATPESGPGSPAGSPTSTEEGTSESATPESGPGSEPATSGS |
| | ETPGTSESATPESGPGTSTEPSEGSAPGTSTEPSEGSAPGTSTEPSEGSAPGTSTEPS |
| | EGSAPGTSTEPSEGSAPGTSTEPSEGSAPGSPAGSPTSTEEGTSTEPSEGSAPGTSES |
| | ATPESGPGSEPATSGSETPGTSESATPESGPGSEPATSGSETPGTSESATPESGPGTS |
| | TEPSEGSAPGTSESATPESGPGSPAGSPTSTEEGSPAGSPTSTEEGSPAGSPTSTEEG |
| | TSESATPESGPGTSTEPSEGSAPGAAEPEA |
| 30 | ASHHHHHHSPAGSPTSTEEGTSESATPESGPGTSTEPSEGSAPGTSESATPESGPGS |
| | EPATSGSETPGTSESATPESGPGSEPATSGSETPGTSESATPESGPGTSTEPSEGSAP |
| | GSPAGSPTSTEEGTSESATPESGPGSEPATSGSETPGTSESATPESGPGSPAGSPTST |
| | EEGSPAGSPTSTEEGTSTEPSEGSAPGTSESATPESGPGTSESATPESGPGTSESATP |
| | ESGPGSEPATSGSETPGSEPATSGSETPGSPAGSPTSTEEGTSTEPSEGSAPGTSTEP |
| | SEGSAPGGSAPQPAHLTFPATSGSETPGTDIQMTQSPSSLSASVGDRVTITCQASQD |
| | ISNYLNWYQQKPGKAPKLLIYDASNLETGVPSRFSGSGSGTDFTFTISSLQPEDIAT |
| | YFCQHFDHLPLAFGGGTKVEIKGATPPETGAETESPGETTGGSAESEPPGEGQVQ |
| | LQESGPGLVKPSETLSLTCTVSGGSVSSGDYYWTWIRQSPGKGLEWIGHIYYSGN |
| | TNYNPSLKSRLTISIDTSKTQFSLKLSSVTAADTAIYYCVRDRVTGAFDIWGQGTM |
| | VTVSSGGGGSELVVTQEPSLTVSPGGTVTLTCRSSTGAVTTSNYANWVQQKPGQ |
| | APRGLIGGTNKRAPGTPARFSGSLLGGKAALTLSGVQPEDEAEYYCALWYSNLW |
| | VFGGGTKLTVLGATPPETGAETESPGETTGGSAESEPPGEGEVQLLESGGGLVQP |
| | GGSLKLSCAASGFTFNTYAMNWVRQAPGKGLEWVARIRSKYNNYATYYADSVK |
| | DRFTISRDDSKNTAYLQMNNLKTEDTAVYYCVRHGNFGNSYVSWFAYWGQGTL |
| | VTVSSGTAEAASASGQPAHLTFPPGSPAGSPTSTEEGTSESATPESGPGTSTEPSEG |
| | SAPGSPAGSPTSTEEGTSTEPSEGSAPGTSTEPSEGSAPGTSESATPESGPGSEPATS |
| | GSETPGSEPATSGSETPGSPAGSPTSTEEGTSESATPESGPGTSTEPSEGSAPGTSTE |
| | PSEGSAPGSPAGSPTSTEEGTSTEPSEGSAPGTSTEPSEGSAPGTSESATPESGPGTS |
| | TEPSEGSAPGTSESATPESGPGSEPATSGSETPGTSTEPSEGSAPGTSTEPSEGSAPG |
| | TSESATPESGPGTSESATPESGPGSPAGSPTSTEEGTSESATPESGPGSEPATSGSET |
| | PGTSESATPESGPGTSTEPSEGSAPGTSTEPSEGSAPGTSTEPSEGSAPGTSTEPSEG |
| | SAPGTSTEPSEGSAPGTSTEPSEGSAPGSPAGSPTSTEEGTSTEPSEGSAPGTSESAT |
| | PESGPGSEPATSGSETPGTSESATPESGPGSEPATSGSETPGTSESATPESGPGTSTE |
| | PSEGSAPGTSESATPESGPGSPAGSPTSTEEGSPAGSPTSTEEGSPAGSPTSTEEGTS |
| | ESATPESGPGTSTEPSEGSAPGAAEPEA |
| 31 | ASSPAGSPTSTESGTSESATPESGPGTSTEPSEGSAPGTSESATPESGPGSEPATSGS |
| | ETPGTSESATPESGPGSTPAESGSETPGTSESATPESGPGTSTEPSEGSAPGSPAGSP |
| | TSTEEGTSESATPESGPGEEPATSGSTPEGTSESATPESGPGSPAGSPTSTEEGSPAG |
| | SPTSTEEGTSTEPSEGSAPGTSESATPESGPGTSESATPESGPGTSESATPESGPGSE |
| | PATSGSETPGSEPATSGSETPGSPAGSPTSTEEGTSTEPSEGSAPGTSTEPSEGSAPG |
| | GSAPGAGRTDNHEPLELGAAATSGSETPGTDIQMTQSPSSLSASVGDRVTITCRAS |
| | QDVNTAVAWYQQKPGKAPKLLIYSASFLYSGVPSRFSGSRSGTDFTLTISSLQPED |
| | FATYYCQQHYTTPPTFGQGTKVEIKGATPPETGAETESPGETTGGSAESEPPGEG |
| | EVQLVESGGGLVQPGGSLRLSCAASGFNIKDTYIHWVRQAPGKGLEWVARIYPT |
| | NGYTRYADSVKGRFTISADTSKNTAYLQMNSLRAEDTAVYYCSRWGGDGFYAM |
| | DYWGQGTLVTVSSGGGSELVVTQEPSLTVSPGGTVTLTCRSSTGAVTTSNYANW |
| | VQQKPGQAPRGLIGGTNKRAPGTPARFSGSSLGGSAALTLSGVQPEDEAEYYCAL |
| | WYSNLWVFGGGTKLTVLGATPPETGAETESPGETTGGSAESEPPGEGEVQLQES |
| | GGGLVQPGGSLKLSCAASGFTFNTYAMNWVRQAPGKGLEWVARIRSKYNNYAT |
| | YYADSVKDRFTISRDDSKNTAYLQMNNLKTEDTAVYYCVRHGNFGNSYVSWFAY |
| | WGQGTLVTVSSGTAEAASASGGAGRTDNHEPLELGAAPGSPAGSPTSTEEGTSES |
| | ATPESGPGTSTEPSEGSAPGSPAGSPTSTEEGTSTEPSEGSAPGTSTEPSEGSAPGTS |
| | ESATPESGPGSEPATSGSETPGSEPATSGSETPGSPAGSPTSTEEGTSESATPESGPG |
| | TSTEPSEGSAPGTSTEPSEGSAPGSPAGSPTSTEEGTSTEPSEGSAPGTSTEPSEGSA |
| | PGTSESATPESGPGTSTEPSEGSAPGTSESATPESGPGSEPATSGSETPGTSTEPSEG |
| | SAPGTSTEPSEGSAPGTSESATPESGPGTSESATPESGPGSPAGSPTSTEEGTSESAT |
| | PESGPGSEPATSGSETPGTSESATPESGPGTSTEPSEGSAPGTSTEPSEGSAPGTSTE |
| | PSEGSAPGTSTEPSEGSAPGTSTEPSEGSAPGTSTEPSEGSAPGSPAGSPTSTEEGTS |
| | TEPSEGSAPGTSESATPESGPGSEPATSGSETPGTSESATPESGPGSEPATSGSETPG |
| | TSESATPESGPGTSTEPSEGSAPGTSESATPESGPGSPAGSPTSTEEGSPAGSPTSTE |
| | EGSPAGSPTSTEEGTSESATPESGPGTSTEPSEGSAPGTSESATPESGPGSEPATSGS |
| | ETPGTSESATPESGPGSEPATSGSETPGTSESATPESGPGTSTEPSEGSAPGSPAGSP |
| | TSTEEGTSESATPESGPGSEPATSGSETPGTSESATPESGPGSPAGSPTSTEEGSPAG |
| | SPTSTEEGTSTEPSEGSAPGTSESATPESGPGTSESATPESGPGftabTSESATPESGP |
| | GSEPATSGPTESGSEPATSGSETPGSPAGSPTSTEEGTSTEPSEGSAPGTESTPSEGS |
| | APGSEPATSGSETPGTSESATPESGPGTSTEPSEGSAPGEPEA |
| 32 | ASHHHHHHSPAGSPTSTEEGTSESATPESGPGTSTEPSEGSAPGTSESATPESGPGS |
| | EPATSGSETPGTSESATPESGPGSEPATSGSETPGTSESATPESGPGTSTEPSEGSAP |
| | GSPAGSPTSTEEGTSESATPESGPGSEPATSGSETPGTSESATPESGPGSPAGSPTST |
| | EEGSPAGSPTSTEEGTSTEPSEGSAPGTSESATPESGPGTSESATPESGPGTSESATP |
| | ESGPGSEPATSGSETPGSEPATSGSETPGSPAGSPTSTEEGTSTEPSEGSAPGTSTEP |
| | SEGSAPGGSAPQPVSLANTATSGSETPGTDIQMTQSPSSLSASVGDRVTITCQASQD |
| | ISNYLNWYQQKPGKAPKLLIYDASNLETGVPSRFSGSGSGTDFTFTISSLQPEDIAT |
| | YFCQHFDHLPLAFGGGTKVEIKGATPPETGAETESPGETTGGSAESEPPGEGQVQ |
| | LQESGPGLVKPSETLSLTCTVSGGSVSSGDYYWTWIRQSPGKGLEWIGHIYYSGN |
| | TNYNPSLKSRLTISIDTSKTQFSLKLSSVTAADTAIYYCVRDRVTGAFDIWGQGTM |
| | VTVSSGGGGSELVVTQEPSLTVSPGGTVTLTCRSSTGAVTTSNYANWVQQKPGQ |
| | APRGLIGGTNKRAPGTPARFSGSLLGGKAALTLSGVQPEDEAEYYCALWYSNLW |
| | VFGGGTKLTVLGATPPETGAETESPGETTGGSAESEPPGEGEVQLLESGGGLVQP |
| | GGSLKLSCAASGFTFNTYAMNWVRQAPGKGLEWVARIRSKYNNYATYYADSVK |
| | DRFTISRDDSKNTAYLQMNNLKTEDTAVYYCVRHGNFGNSYVSWFAYWGQGTL |
| | VTVSSGTAEAASASGQPVSLANTPGSPAGSPTSTEEGTSESATPESGPGTSTEPSEG |
| | SAPGSPAGSPTSTEEGTSTEPSEGSAPGTSTEPSEGSAPGTSESATPESGPGSEPATS |
| | GSETPGSEPATSGSETPGSPAGSPTSTEEGTSESATPESGPGTSTEPSEGSAPGTSTE |
| | PSEGSAPGSPAGSPTSTEEGTSTEPSEGSAPGTSTEPSEGSAPGTSESATPESGPGTS |
| | TEPSEGSAPGTSESATPESGPGSEPATSGSETPGTSTEPSEGSAPGTSTEPSEGSAPG |
| | TSESATPESGPGTSESATPESGPGSPAGSPTSTEEGTSESATPESGPGSEPATSGSET |
| | PGTSESATPESGPGTSTEPSEGSAPGTSTEPSEGSAPGTSTEPSEGSAPGTSTEPSEG |
| | SAPGTSTEPSEGSAPGTSTEPSEGSAPGSPAGSPTSTEEGTSTEPSEGSAPGTSESAT |
| | PESGPGSEPATSGSETPGTSESATPESGPGSEPATSGSETPGTSESATPESGPGTSTE |
| | PSEGSAPGTSESATPESGPGSPAGSPTSTEEGSPAGSPTSTEEGSPAGSPTSTEEGTS |
| | ESATPESGPGTSTEPSEGSAPGAAEPEA |
| 33 | ASHHHHHHSPAGSPTSTEEGTSESATPESGPGTSTEPSEGSAPGTSESATPESGPGS |
| | EPATSGSETPGTSESATPESGPGSEPATSGSETPGTSESATPESGPGTSTEPSEGSAP |
| | GSPAGSPTSTEEGTSESATPESGPGSEPATSGSETPGTSESATPESGPGSPAGSPTST |
| | EEGSPAGSPTSTEEGTSTEPSEGSAPGTSESATPESGPGTSESATPESGPGTSESATP |
| | ESGPGSEPATSGSETPGSEPATSGSETPGSPAGSPTSTEEGTSTEPSEGSAPGTSTEP |
| | SEGSAPGGSAPGPSGHMGRATSGSETPGTDIQMTQSPSSLSASVGDRVTITCQASQ |
| | DISNYLNWYQQKPGKAPKLLIYDASNLETGVPSRFSGSGSGTDFTFTISSLQPEDIA |
| | TYFCQHFDHLPLAFGGGTKVEIKGATPPETGAETESPGETTGGSAESEPPGEGQV |
| | QLQESGPGLVKPSETLSLTCTVSGGSVSSGDYYWTWIRQSPGKGLEWIGHIYYSG |
| | NTNYNPSLKSRLTISIDTSKTQFSLKLSSVTAADTAIYYCVRDRVTGAFDIWGQGT |
| | MVTVSSGGGGSELVVTQEPSLTVSPGGTVTLTCRSSTGAVTTSNYANWVQQKPG |
| | QAPRGLIGGTNKRAPGTPARFSGSLLGGKAALTLSGVQPEDEAEYYCALWYSNL |
| | WVFGGGTKLTVLGATPPETGAETESPGETTGGSAESEPPGEGEVQLLESGGGLV |
| | QPGGSLKLSCAASGFTFNTYAMNWVRQAPGKGLEWVARIRSKYNNYATYYADS |
| | VKDRFTISRDDSKNTAYLQMNNLKTEDTAVYYCVRHGNFGNSYVSWFAYWGQG |
| | TLVTVSSGTAEAASASGGPSGHMGRPGSPAGSPTSTEEGTSESATPESGPGTSTEPS |
| | EGSAPGSPAGSPTSTEEGTSTEPSEGSAPGTSTEPSEGSAPGTSESATPESGPGSEPA |
| | TSGSETPGSEPATSGSETPGSPAGSPTSTEEGTSESATPESGPGTSTEPSEGSAPGTS |
| | TEPSEGSAPGSPAGSPTSTEEGTSTEPSEGSAPGTSTEPSEGSAPGTSESATPESGPG |
| | TSTEPSEGSAPGTSESATPESGPGSEPATSGSETPGTSTEPSEGSAPGTSTEPSEGSA |
| | PGTSESATPESGPGTSESATPESGPGSPAGSPTSTEEGTSESATPESGPGSEPATSGS |
| | ETPGTSESATPESGPGTSTEPSEGSAPGTSTEPSEGSAPGTSTEPSEGSAPGTSTEPS |
| | EGSAPGTSTEPSEGSAPGTSTEPSEGSAPGSPAGSPTSTEEGTSTEPSEGSAPGTSES |
| | ATPESGPGSEPATSGSETPGTSESATPESGPGSEPATSGSETPGTSESATPESGPGTS |
| | TEPSEGSAPGTSESATPESGPGSPAGSPTSTEEGSPAGSPTSTEEGSPAGSPTSTEEG |
| | TSESATPESGPGTSTEPSEGSAPGAAEPEA |
| 34 | ASHHHHHHSPAGSPTSTEEGTSESATPESGPGTSTEPSEGSAPGTSESATPESGPGS |
| | EPATSGSETPGTSESATPESGPGSEPATSGSETPGTSESATPESGPGTSTEPSEGSAP |
| | GSPAGSPTSTEEGTSESATPESGPGSEPATSGSETPGTSESATPESGPGSPAGSPTST |
| | EEGSPAGSPTSTEEGTSTEPSEGSAPGTSESATPESGPGTSESATPESGPGTSESATP |
| | ESGPGSEPATSGSETPGSEPATSGSETPGSPAGSPTSTEEGTSTEPSEGSAPGTSTEP |
| | SEGSAPGGSAPGVRGLTGPATSGSETPGTDIQMTQSPSSLSASVGDRVTITCQASQ |
| | DISNYLNWYQQKPGKAPKLLIYDASNLETGVPSRFSGSGSGTDFTFTISSLQPEDIA |
| | TYFCQHFDHLPLAFGGGTKVEIKGATPPETGAETESPGETTGGSAESEPPGEGQV |
| | QLQESGPGLVKPSETLSLTCTVSGGSVSSGDYYWTWIRQSPGKGLEWIGHIYYSG |
| | NTNYNPSLKSRLTISIDTSKTQFSLKLSSVTAADTAIYYCVRDRVTGAFDIWGQGT |
| | MVTVSSGGGGSELVVTQEPSLTVSPGGTVTLTCRSSTGAVTTSNYANWVQQKPG |
| | QAPRGLIGGTNKRAPGTPARFSGSLLGGKAALTLSGVQPEDEAEYYCALWYSNL |
| | WVFGGGTKLTVLGATPPETGAETESPGETTGGSAESEPPGEGEVQLLESGGGLV |
| | QPGGSLKLSCAASGFTFNTYAMNWVRQAPGKGLEWVARIRSKYNNYATYYADS |
| | VKDRFTISRDDSKNTAYLQMNNLKTEDTAVYYCVRHGNFGNSYVSWFAYWGQG |
| | TLVTVSSGTAEAASASGGVRGLTGPPGSPAGSPTSTEEGTSESATPESGPGTSTEPS |
| | EGSAPGSPAGSPTSTEEGTSTEPSEGSAPGTSTEPSEGSAPGTSESATPESGPGSEPA |
| | TSGSETPGSEPATSGSETPGSPAGSPTSTEEGTSESATPESGPGTSTEPSEGSAPGTS |
| | TEPSEGSAPGSPAGSPTSTEEGTSTEPSEGSAPGTSTEPSEGSAPGTSESATPESGPG |
| | TSTEPSEGSAPGTSESATPESGPGSEPATSGSETPGTSTEPSEGSAPGTSTEPSEGSA |
| | PGTSESATPESGPGTSESATPESGPGSPAGSPTSTEEGTSESATPESGPGSEPATSGS |
| | ETPGTSESATPESGPGTSTEPSEGSAPGTSTEPSEGSAPGTSTEPSEGSAPGTSTEPS |
| | EGSAPGTSTEPSEGSAPGTSTEPSEGSAPGSPAGSPTSTEEGTSTEPSEGSAPGTSES |
| | ATPESGPGSEPATSGSETPGTSESATPESGPGSEPATSGSETPGTSESATPESGPGTS |
| | TEPSEGSAPGTSESATPESGPGSPAGSPTSTEEGSPAGSPTSTEEGSPAGSPTSTEEG |
| | TSESATPESGPGTSTEPSEGSAPGAAEPEA |
| 35 | ASHHHHHHSPAGSPTSTEEGTSESATPESGPGTSTEPSEGSAPGTSESATPESGPGS |
| | EPATSGSETPGTSESATPESGPGSEPATSGSETPGTSESATPESGPGTSTEPSEGSAP |
| | GSPAGSPTSTEEGTSESATPESGPGSEPATSGSETPGTSESATPESGPGSPAGSPTST |
| | EEGSPAGSPTSTEEGTSTEPSEGSAPGTSESATPESGPGTSESATPESGPGTSESATP |
| | ESGPGSEPATSGSETPGSEPATSGSETPGSPAGSPTSTEEGTSTEPSEGSAPGTSTEP |
| | SEGSAPGGSAPHPVELLARATSGSETPGTDIQMTQSPSSLSASVGDRVTITCKASQ |
| | DVSIGVAWYQQKPGKAPKLLIYSASYRYTGVPSRFSGSGSGTDFTLTISSLQPEDF |
| | ATYYCQQYYTYPYTFGQGTKVEIKGATPPETGAETESPGETTGGSAESEPPGEGE |
| | VQLVESGGGLVQPGGSLRLSCAASGFTFTDYTMDWVRQAPGKGLEWVADVNPN |
| | SGGSIYNQRFKGRFTLSVDRSKNTLYLQMNSLRAEDTAVYYCARNLGPSFYFDY |
| | WGQGTLVTVSSGGGGSELVVTQEPSLTVSPGGTVTLTCRSSNGAVTSSNYANWV |
| | QQKPGQAPRGLIGGTNKRAPGTPARFSGSLLGGKAALTLSGVQPEDEAVYYCAL |
| | WYPNLWVFGGGTKLTVLGATPPETGAETESPGETTGGSAESEPPGEGEVQLLES |
| | GGGIVQPGGSLKLSCAASGFTFNTYAMNWVRQAPGKGLEWVARIRSKYNNYAT |
| | YYADSVKDRFTISRDDSKNTVYLQMNNLKTEDTAVYYCVRHENFGNSYVSWFAH |
| | WGQGTLVTVSSGTAEAASASGHPVELLARPGSPAGSPTSTEEGTSESATPESGPGT |
| | STEPSEGSAPGSPAGSPTSTEEGTSTEPSEGSAPGTSTEPSEGSAPGTSESATPESGP |
| | GSEPATSGSETPGSEPATSGSETPGSPAGSPTSTEEGTSESATPESGPGTSTEPSEGS |
| | APGTSTEPSEGSAPGSPAGSPTSTEEGTSTEPSEGSAPGTSTEPSEGSAPGTSESATP |
| | ESGPGTSTEPSEGSAPGTSESATPESGPGSEPATSGSETPGTSTEPSEGSAPGTSTEP |
| | SEGSAPGTSESATPESGPGTSESATPESGPGSPAGSPTSTEEGTSESATPESGPGSEP |
| | ATSGSETPGTSESATPESGPGTSTEPSEGSAPGTSTEPSEGSAPGTSTEPSEGSAPGT |
| | STEPSEGSAPGTSTEPSEGSAPGTSTEPSEGSAPGSPAGSPTSTEEGTSTEPSEGSAP |
| | GTSESATPESGPGSEPATSGSETPGTSESATPESGPGSEPATSGSETPGTSESATPES |
| | GPGTSTEPSEGSAPGTSESATPESGPGSPAGSPTSTEEGSPAGSPTSTEEGSPAGSPT |
| | STEEGTSESATPESGPGTSTEPSEGSAPGAAEPEA |

### Target Tissues or Cells

In some embodiments of the compositions (such as the therapeutic agents, or activatable therapeutic agents described hereinabove) or methods described herein, the target tissue or cell can contain therein or thereon, or can be associated with in proximity thereto, a reporter polypeptide (such as one described herein this TARGET TISSUES OR CELLS section) capable of being cleaved by a mammalian protease at a cleavage sequence (such as one set forth in Table A). The reporter polypeptide can be a polypeptide set forth in the “Report Protein” column of Table A (or any subset thereof). In some embodiments, the reporter polypeptide can be selected from coagulation factor, complement component, tubulin, immunoglobulin, apolipoprotein, serum amyloid, insulin, growth factor, fibrinogen, PDZ domain protein, LIM domain protein, c-reactive protein, serum albumin, versican, collagen, elastin, keratin, kininogen-1, alpha-2-antiplasmin, clusterin, biglycan, alpha-1-antitrypsin, transthyretin, alpha-1-antichymotrypsin, glucagon, hepcidin, thymosin beta-4, haptoglobin, hemoglobin subunit alpha, caveolae-associated protein 2, alpha-2-HS-glycoprotein, chromogranin-A, vitronectin, hemopexin, epididymis secretory sperm binding protein, secretogranin-2, angiotensinogen, transgelin-2, pancreatic prohormone, neurosecretory protein VGF, ceruloplasmin, PDZ and LIM domain protein 1, multimerin-1, inter-alpha-trypsin inhibitor heavy chain H2, N-acetylmuramoyl-L-alanine amidase, histone H1.4, adhesion G-protein coupled receptor G6, mannan-binding lectin serine protease 2, prothrombin, deleted in malignant brain tumors 1 protein, desmoglein-3, calsyntenin-1, alpha-2-macroglobulin, myosin-9, sodium/potassium-transporting ATPase subunit gamma, oncoprotein-induced transcript 3 protein, serglycin, histidine-rich glycoprotein, inter-alpha-trypsin inhibitor heavy chain H5, integrin alpha-IIb, membrane-associated progesterone receptor component 1, histone H1.2, rho GDP-dissociation inhibitor 2, zinc-alpha-2-glycoprotein, talin-1, secretogranin-1, neutrophil defensin 3, cytochrome P450 2E1, gastric inhibitory polypeptide, transcription initiation factor TFIID subunit 1, integral membrane protein 2B, pigment epithelium-derived factor, voltage-dependent N-type calcium channel subunit alpha-1B, ras GTPase-activating protein nGAP, type I cytoskeletal 17, sulfhydryl oxidase 1, homeobox protein Hox-B2, transcription factor SOX-10, E3 ubiquitin-protein ligase SIAH2, decorin, secreted protein acidic and rich in cysteine (SPARC), laminin gamma 1 chain, vimentin, and nidogen-1 (NID1). In some embodiments, the reporter polypeptide can be selected from collagen, elastin, keratin, coagulation factor, complement component, tubulin, immunoglobulin, apolipoprotein, serum amyloid, insulin, growth factor, fibrinogen, PDZ domain protein, LIM domain protein, c-reactive protein, and serum albumin. The collagen can comprise alpha chain(s) (such as alpha-1, alpha-2, alpha-3, or a combination thereof) of collagen type I, collagen type II, collagen type III, collagen type IV, collagen type V, collagen type VI, collagen type VII, collagen type VIII, collagen type IX, collagen type X, collagen type XI, collagen type XII, collagen type XIII, collagen type XIV, collagen type XV, collagen type XVI, collagen type XVII, collagen type XVIII, collagen type XIX, collagen type XX, collagen type XXI, collagen type XXII, collagen type XXIII, collagen type XXIV, collagen type XXV, collagen type XXVI, collagen type XXVII, collagen type XXVIII, collagen type XXIX, or a combination thereof. The coagulation factor can be selected from coagulation factor IX, coagulation factor XII, and coagulation factor XIII A chain. The complement component can be selected from C1 (for example, and not limited to, complement C1r subcomponent-like protein, complement C1r subcomponent), C3, C4 (for example, and not limited to, complement C4-A, complement C4-B), and C5. The tubulin can be selected from tubulin alpha chain (for example, and not limited to, tubulin alpha-4A chain), and tubulin beta chain. The immunoglobulin can be selected from immunoglobulin lambda variable 3-21, immunoglobulin lambda variable 3-25, immunoglobulin lambda variable 1-51, immunoglobulin lambda variable 1-36, immunoglobulin kappa variable 3-20, immunoglobulin kappa variable 2-30, probable non-functional immunoglobulin kappa variable 2D-24, immunoglobulin lambda constant 3, immunoglobulin kappa variable 2-28, immunoglobulin kappa variable 3-11, immunoglobulin kappa variable 1-39, immunoglobulin lambda variable 6-57, immunoglobulin kappa variable 3-15, immunoglobulin lambda variable 2-18, immunoglobulin heavy variable 3-15, immunoglobulin lambda variable 2-11, immunoglobulin lambda variable 3-27, and immunoglobulin kappa variable 4-1. The apolipoprotein can be selected from apolipoprotein A-I, apolipoprotein A-I Isoform 1, apolipoprotein apolipoprotein C-I, apolipoprotein A-II, and apolipoprotein L1. The serum amyloid protein can be selected from serum amyloid A-1 protein, and serum amyloid A-2 protein. The growth factor can be selected from insulin-like growth factor II, latent-transforming growth factor beta-binding protein 2, and latent-transforming growth factor beta-binding protein 4. The fibrinogen can be selected from fibrinogen alpha chain, fibrinogen beta chain, and fibrinogen gamma chain. The LIM domain protein can be zyxin. In some embodiments, the reporter polypeptide can be selected from the group consisting of versican, type II collagen alpha-1 chain, kininogen-1, complement C4-A, complement C4-B, complement C3, alpha-2-antiplasmin, clusterin, biglycan, elastin, fibrinogen alpha chain, alpha-1-antitrypsin, fibrinogen beta chain, type III collagen alpha-1 chain, serum amyloid A-1 protein, transthyretin, apolipoprotein A-I, apolipoprotein A-I Isoform 1, alpha-1-antichymotrypsin, glucagon, hepcidin, serum amyloid A-2 protein, thymosin beta-4, haptoglobin, hemoglobin subunit alpha, caveolae-associated protein 2, alpha-2-HS-glycoprotein, chromogranin-A, vitronectin, hemopexin, epididymis secretory sperm binding protein, zyxin, apolipoprotein secretogranin-2, angiotensinogen, c-reactive protein, serum albumin, transgelin-2, pancreatic prohormone, neurosecretory protein VGF, ceruloplasmin, PDZ and LIM domain protein 1, tubulin alpha-4A chain, multimerin-1, inter-alpha-trypsin inhibitor heavy chain H2, apolipoprotein C-I, fibrinogen gamma chain, N-acetylmuramoyl-L-alanine amidase, immunoglobulin lambda variable 3-21, histone H1.4, adhesion G-protein coupled receptor G6, immunoglobulin lambda variable 3-25, immunoglobulin lambda variable 1-51, immunoglobulin lambda variable 1-36, mannan-binding lectin serine protease 2, immunoglobulin kappa variable 3-20, immunoglobulin kappa variable 2-30, insulin-like growth factor II, apolipoprotein A-II, probable non-functional immunoglobulin kappa variable 2D-24, prothrombin, coagulation factor IX, apolipoprotein L1, deleted in malignant brain tumors 1 protein, desmoglein-3, calsyntenin-1, immunoglobulin lambda constant 3, complement C5, alpha-2-macroglobulin, myosin-9, sodium/potassium-transporting ATPase subunit gamma, immunoglobulin kappa variable 2-28, oncoprotein-induced transcript 3 protein, serglycin, coagulation factor XII, coagulation factor XIII A chain, insulin, histidine-rich glycoprotein, immunoglobulin kappa variable 3-11, immunoglobulin kappa variable 1-39, collagen alpha-1(I) chain, inter-alpha-trypsin inhibitor heavy chain H5, latent-transforming growth factor beta-binding protein 2, integrin alpha-IIb, membrane-associated progesterone receptor component 1, immunoglobulin lambda variable 6-57, immunoglobulin kappa variable 3-15, complement C1r subcomponent-like protein, histone H1.2, rho GDP-dissociation inhibitor 2, latent-transforming growth factor beta-binding protein 4, collagen alpha-1(XVIII) chain, immunoglobulin lambda variable 2-18, zinc-alpha-2-glycoprotein, talin-1, secretogranin-1, neutrophil defensin 3, cytochrome P450 2E1, gastric inhibitory polypeptide, immunoglobulin heavy variable 3-15, immunoglobulin lambda variable 2-11, transcription initiation factor TFIID subunit 1, collagen alpha-1(VII) chain, integral membrane protein 2B, pigment epithelium-derived factor, voltage-dependent N-type calcium channel subunit alpha-1B, immunoglobulin lambda variable 3-27, ras GTPase-activating protein nGAP, keratin, type I cytoskeletal 17, tubulin beta chain, sulfhydryl oxidase 1, immunoglobulin kappa variable 4-1, complement C1r subcomponent, homeobox protein Hox-B2, transcription factor SOX-10, E3 ubiquitin-protein ligase SIAH2, decorin, SPARC, type I collagen alpha-1 chain, type IV collagen alpha-1 chain, laminin gamma 1 chain, vimentin, type III collagen, type IV collagen alpha-3 chain, type VII collagen alpha-1 chain, type VI collagen alpha-1 chain, type V collagen alpha-1 chain, nidogen-1, and type VI collagen alpha-3 chain. In some embodiments, the reporter polypeptide can comprise a cleavage sequence set forth in Column II or III of Table A (or a subset thereof) and/or the group set forth in Tables 1(a)-1(j) (or any subset thereof). The reporter polypeptide can comprise a sequence set forth in Column IV of Table A (or a subset thereof). The reporter polypeptide can comprise a sequence set forth in Column V of Table A (or a subset thereof). The reporter polypeptide can comprise a sequence set forth in Column VI of Table A (or a subset thereof). The reporter polypeptide can comprise a peptide biomarker (or a peptide biomarker sequence) (such as one shown in Table A) capable of being identified from a biological sample of the subject. The peptide biomarker can comprise a sequence set forth in Column IV of Table A (or a subset thereof). The peptide biomarker can comprise a sequence set forth in Column V of Table A (or a subset thereof). The peptide biomarker can comprise a sequence set forth in Column VI of Table A (or a subset thereof). In some embodiments, the reporter polypeptide is selected from the group set forth in Column I of Table A (or a subset thereof). In some embodiments, the cleavage sequence of the reporter polypeptide does not comprise a methionine residue immediately N-terminal to a scissile bond (contained therein), when the methionine is the first residue at N terminus of the reporter polypeptide.

TABLE A
Exemplary cleavage sequences and biomarker sequences in exemplary
reporter polypeptides

| | | Column II | | Column III | | | | | | | |
|---|---|---|---|---|---|---|---|---|---|---|---|
| | | | Clea- | | Clea- | | | | | | |
| | Column I | | vage | | vage | Column IV | | Column V | | Column VI | |
| | Reporter | SEQ | Se- | SEQ | Se- | SEQ | N- | SEQ | | SEQ | C- |
| | Poly- | ID | quence | ID | quence | ID | terminal | ID | Center | ID | terminal |
| # | peptide | NO: | 1* | NO: | 2 | NO: | Fragment | NO: | Fragment | NO: | Fragment |
| 1 | type III | 755 | GPPG- | | N/A | 1688 | GGPGPQGPPG | | N/A | 2598 | KNGETGPQGP |
| | collagen | | KNGE | | | | | | | | |
| | alpha-1 | | | | | | | | | | |
| | chain | | | | | | | | | | |
| 2 | versican | 756 | VENA- | | N/A | 1689 | CGQPPVVENA | | N/A | 2599 | KTFGKMKPRY |
| | | | KTFG | | | | | | | | |
| 3 | type II | 757 | GAAG- | | N/A | 1690 | GPPGRDGAAG | | N/A | 2600 | VKGDRGETGA |
| | collagen | | VKGD | | | | | | | | |
| | alpha-1 | | | | | | | | | | |
| | chain | | | | | | | | | | |
| 4 | kininogen- | 758 | SLMK- | 1291 | FSPF- | 1691 | QPLGMISLMK | 2199 | RPPGFSPF | 2601 | RSSRIGEIKE |
| | 1 | | RPPG | | RSSR | | | | | | |
| 5 | complement | 759 | NGFK- | 1292 | LNNR- | 1692 | LSSTGRNGFK | 2200 | SHALQLNNR | 2602 | QIRGLEEELQ |
| | C4-A OR | | SHAL | | QIRG | | | | | | |
| | complement | | | | | | | | | | |
| | C4-B | | | | | | | | | | |
| 6 | kininogen- | 760 | SLMK- | 1293 | SPFR- | 1693 | QPLGMISLMK | 2201 | RPPGFSPFR | 2603 | SSRIGEIKEE |
| | 1 | | RPPG | | SSRI | | | | | | |
| 7 | complement | 761 | THRI- | 1294 | SLLR- | 1694 | SRSSKITHRI | 2202 | HWESASLLR | 2604 | SEETKENEGF |
| | C3 | | HWES | | SEET | | | | | | |
| 8 | complement | 762 | SSKI- | 1295 | WESA- | 1695 | LQLPSRSSKI | 2203 | THRIHWESA | 2605 | SLLRSEETKE |
| | C3 | | THRI | | SLLR | | | | | | |
| 9 | complement | 763 | KSHA- | 1296 | RQIR- | 1696 | TGRNGFKSHA | 2204 | LQLNNRQIR | 2606 | GLEEELQFSL |
| | C4-A OR | | LQLN | | GLEE | | | | | | |
| | complement | | | | | | | | | | |
| | C4-B | | | | | | | | | | |
| 10 | complement | 764 | ITHR- | 1297 | SLLR- | 1697 | PSRSSKITHR | 2205 | IHWESASLLR | 2607 | SEETKENEGF |
| | C3 | | IHWE | | SEET | | | | | | |
| 11 | complement | 765 | FKSH- | 1298 | RQIR- | 1698 | STGRNGFKSH | 2206 | ALQLNNRQIR | 2608 | GLEEELQFSL |
| | C4-A OR | | ALQL | | GLEE | | | | | | |
| | complement | | | | | | | | | | |
| | C4-B | | | | | | | | | | |
| 12 | complement | 766 | RSSK- | 1299 | WESA- | 1699 | SLQLPSRSSK | 2207 | ITHRIHWESA | 2609 | SLLRSEETKE |
| | C3 | | ITHR | | SLLR | | | | | | |
| 13 | alpha-2- | 767 | PVSA- | 1300 | TSGP- | 1700 | PCSVFSPVSA | 2208 | MEPLGRQLTSGP | 2610 | NQEQVSPLTL |
| | anti- | | MEPL | | NQEQ | | | | | | |
| | plasmin | | | | | | | | | | |
| 14 | kininogen- | 768 | GHTR- | 1301 | EKQR- | 1701 | DSGKEQGHTR | 2209 | RHDWGHEKQR | 2611 | KHNLGHGHKH |
| | 1 | | RHDW | | KHNL | | | | | | |
| 15 | complement | 769 | KITH- | 1302 | SLLR- | 1702 | LPSRSSKITH | 2210 | RIHWESASLLR | 2612 | SEETKENEGF |
| | C3 | | RIHW | | SEET | | | | | | |
| 16 | complement | 770 | SRSS- | 1303 | WESA- | 1703 | VSLQLPSRSS | 2211 | KITHRIHWESA | 2613 | SLLRSEETKE |
| | C3 | | KITH | | SLLR | | | | | | |
| 17 | complement | 771 | RQIR- | 1304 | LGSK- | 1704 | ALQLNNRQIR | 2212 | GLEEELQFSLGS | 2614 | INVKVGGNSK |
| | C4-A OR | | GLEE | | INVK | | | | K | | |
| | complement | | | | | | | | | | |
| | C4-B | | | | | | | | | | |
| 18 | complement | 772 | NGFK- | 1305 | RQIR- | 1705 | LSSTGRNGFK | 2213 | SHALQLNNRQIR | 2615 | GLEEELQFSL |
| | C4-A OR | | SHAL | | GLEE | | | | | | |
| | complement | | | | | | | | | | |
| | C4-B | | | | | | | | | | |
| 19 | complement | 773 | PSRS- | 1306 | WESA- | 1706 | DVSLQLPSRS | 2214 | SKITHRIHWESA | 2616 | SLLRSEETKE |
| | C3 | | SKIT | | SLLR | | | | | | |
| 20 | complement | 774 | STGR- | 1307 | LNNR- | 1707 | LNVTLSSTGR | 2215 | NGFKSHALQLNN | 2617 | QIRGLEEELQ |
| | C4-A OR | | NGFK | | QIRG | | | | R | | |
| | complement | | | | | | | | | | |
| | C4-B | | | | | | | | | | |
| 21 | clusterin | 775 | LPHR- | 1308 | SRIV- | 1708 | HYLPFSLPHR | 2216 | RPHFFFPKSRIV | 2618 | RSLMPFSPYE |
| | | | RPHF | | RSLM | | | | | | |
| 22 | biglycan | 776 | NNPVP- | | N/A | 1709 | GISLENNPVP | | N/A | 2619 | YWEVQPATFR |
| | | | YWEVQ | | | | | | | | |
| 23 | elastin | 777 | GLPYT- | | N/A | 1710 | LPGGYGLPYT | | N/A | 2620 | TGKLPYGYGP |
| | | | TGKLP | | | | | | | | |
| 24 | elastin | 778 | ARPGF- | | N/A | 1711 | LGGVAARPGF | | N/A | 2621 | GLSPIFPGGA |
| | | | GLSPI | | | | | | | | |
| 25 | fibrinogen | 779 | SRGK- | 1309 | ESKS- | 1712 | GIAEFPSRGK | 2217 | SSSYSKQFTSST | 2622 | YKMADEAGSE |
| | alpha | | SSSY | | YKMA | | | | SYNRGDSTFESK | | |
| | chain | | | | | | | | S | | |
| 26 | fibrinogen | 780 | SRGK- | 1310 | SKSY- | 1713 | GIAEFPSRGK | 2218 | SSSYSKQFTSST | 2623 | KMADEAGSEA |
| | alpha | | SSSY | | KMAD | | | | SYNRGDSTFESK | | |
| | chain | | | | | | | | SY | | |
| 27 | fibrinogen | 781 | DGFR- | 1311 | PSRG- | 1714 | SGIGTLDGFR | 2219 | HRHPDEAAFFDT | 2624 | KSSSYSKQFT |
| | alpha | | HRHP | | KSSS | | | | ASTGKTFPGFFS | | |
| | chain | | | | | | | | PMLGEFVSETES | | |
| | | | | | | | | | RGSESGIFTNTK | | |
| | | | | | | | | | ESSSHHPGIAEF | | |
| | | | | | | | | | PSRG | | |
| 28 | fibrinogen | 782 | SRGK- | 1312 | SYKM- | 1715 | GIAEFPSRGK | 2220 | SSSYSKQFTSST | 2625 | ADEAGSEADH |
| | alpha | | SSSY | | ADEA | | | | SYNRGDSTFESK | | |
| | chain | | | | | | | | SYKM | | |
| 29 | fibrinogen | 783 | TAWT- | 1313 | GGVR- | 1716 | VLSVVGTAWT | 2221 | ADSGEGDFLAEG | 2626 | GPRVVERHQS |
| | alpha | | ADSG | | GPRV | | | | GGVR | | |
| | chain | | | | | | | | | | |
| 30 | fibrinogen | 784 | AWTA- | 1314 | GGVR- | 1717 | LSVVGTAWTA | 2222 | DSGEGDFLAEGG | 2627 | GPRVVERHQS |
| | alpha | | DSGE | | GPRV | | | | GVR | | |
| | chain | | | | | | | | | | |
| 31 | fibrinogen | 785 | AWTA- | 1315 | GGGV- | 1718 | LSVVGTAWTA | 2223 | DSGEGDFLAEGG | 2628 | RGPRVVERHQ |
| | alpha | | DSGE | | RGPR | | | | GV | | |
| | chain | | | | | | | | | | |
| 32 | fibrinogen | 786 | KNNK- | 1316 | ANNR- | 1719 | SLFEYQKNNK | 2224 | DSHSLTTNIMEI | 2629 | DNTYNRVSED |
| | alpha | | DSHS | | DNTY | | | | LRGDFSSANNR | | |
| | chain | | | | | | | | | | |
| 33 | fibrinogen | 787 | WTAD- | 788 | GGVR- | 1720 | SVVGTAWTAD | 2225 | SGEGDFLAEGGG | 2630 | GPRVVERHQS |
| | alpha | | SGEG | | GPRV | | | | VR | | |
| | chain | | | | | | | | | | |
| 34 | fibrinogen | 789 | SRGK- | 1317 | YKMA- | 1721 | GIAEFPSRGK | 2226 | SSSYSKQFTSST | 2631 | DEAGSEADHE |
| | alpha | | SSSY | | DEAG | | | | SYNRGDSTFESK | | |
| | chain | | | | | | | | SYKMA | | |
| 35 | fibrinogen | 790 | TAWT- | 1318 | GGGV- | 1722 | VLSVVGTAWT | 2227 | ADSGEGDFLAEG | 2632 | RGPRVVERHQ |
| | alpha | | ADSG | | RGPR | | | | GGV | | |
| | chain | | | | | | | | | | |
| 36 | fibrinogen | 791 | GNFK- | 1319 | MRME- | 1723 | VPDLVPGNFK | 2228 | SQLQKVPPEWKA | 2633 | LERPGGNEIT |
| | alpha | | SQLQ | | LERP | | | | LTDMPQMRME | | |
| | chain | | | | | | | | | | |
| 37 | fibrinogen | 792 | TADS- | 1320 | GGVR- | 1724 | VVGTAWTADS | 2229 | GEGDFLAEGGGV | 2634 | GPRVVERHQS |
| | alpha | | GEGD | | GPRV | | | | R | | |
| | chain | | | | | | | | | | |
| 38 | fibrinogen | 793 | SSGP- | 1321 | SSGP- | 1725 | GSWNSGSSGP | 2230 | GSTGNRNPGSSG | 2635 | GSTGSWNSGS |
| | alpha | | GSTG | | GSTG | | | | TGGTATWKPGSS | | |
| | chain | | | | | | | | GP | | |
| 39 | fibrinogen | 794 | ADSG- | 1322 | GGVR- | 1726 | VGTAWTADSG | 2231 | EGDFLAEGGGVR | 2636 | GPRVVERHQS |
| | alpha | | EGDF | | GPRV | | | | | | |
| | chain | | | | | | | | | | |
| 40 | fibrinogen | 795 | SRGK- | 1323 | YKMA- | 1727 | GIAEFPSRGK | 2232 | SSSYSKQFTSST | 2637 | DEAGSEADHE |
| | alpha | | SSSY | | DEAG | | | | SYNRGDSTFESK | | |
| | chain | | | | | | | | SYKMA | | |
| 41 | fibrinogen | 796 | SRGK- | 1324 | TFES- | 1728 | GIAEFPSRGK | 2233 | SSSYSKQFTSST | 2638 | KSYKMADEAG |
| | alpha | | SSSY | | KSYK | | | | SYNRGDSTFES | | |
| | chain | | | | | | | | | | |
| 42 | fibrinogen | 797 | TAWT- | 1325 | PRVV- | 1729 | VLSVVGTAWT | 2234 | ADSGEGDFLAEG | 2639 | ERHQSACKDS |
| | alpha | | ADSG | | ERHQ | | | | GGVRGPRVV | | |
| | chain | | | | | | | | | | |
| 43 | fibrinogen | 798 | NFKS- | 1326 | PEWK- | 1730 | PDLVPGNFKS | 2235 | QLQKVPPEWK | 2640 | ALTDMPQMRM |
| | alpha | | QLQK | | ALTD | | | | | | |
| | chain | | | | | | | | | | |
| 44 | fibrinogen | 799 | KMKP- | 1327 | GNFK- | 1731 | QHLPLIKMKP | 2236 | VPDLVPGNFK | 2641 | SQLQKVPPEW |
| | alpha | | VPDL | | SQLQ | | | | | | |
| | chain | | | | | | | | | | |
| 45 | fibrinogen | 800 | SGEG- | 1328 | GGVR- | 1732 | TAWTADSGEG | 2237 | DFLAEGGGVR | 2642 | GPRVVERHQS |
| | alpha | | DFLA | | GPRV | | | | | | |
| | chain | | | | | | | | | | |
| 46 | fibrinogen | 801 | TAWT- | 1329 | GPRV- | 1733 | VLSVVGTAWT | 2238 | ADSGEGDFLAEG | 2643 | VERHQSACKD |
| | alpha | | ADSG | | VERH | | | | GGVRGPRV | | |
| | chain | | | | | | | | | | |
| 47 | fibrinogen | 802 | STGK- | 1330 | PSRG- | 1734 | AFFDTASTGK | 2239 | TFPGFFSPMLGE | 2644 | KSSSYSKQFT |
| | alpha | | TFPG | | KSSS | | | | FVSETESRGSES | | |
| | chain | | | | | | | | GIFTNTKESSSH | | |
| | | | | | | | | | HPGIAEFPSRG | | |
| 48 | fibrinogen | 803 | PLIK- | 1331 | GNFK- | 1735 | RDRQHLPLIK | 2240 | MKPVPDLVPGNF | 2645 | SQLQKVPPEW |
| | alpha | | MKPV | | SQLQ | | | | K | | |
| | chain | | | | | | | | | | |
| 49 | fibrinogen | 804 | PLIK- | 1332 | SQLQ- | 1736 | RDRQHLPLIK | 2241 | MKPVPDLVPGNF | 2646 | KVPPEWKALT |
| | alpha | | MKPV | | KVPP | | | | KSQLQ | | |
| | chain | | | | | | | | | | |
| 50 | fibrinogen | 805 | GSWN- | 1333 | SSGP- | 1737 | RNPSSAGSWN | 2242 | SGSSGPGSTGNR | 2647 | GSTGSWNSGS |
| | alpha | | SGSS | | GSTG | | | | NPGSSGTGGTAT | | |
| | chain | | | | | | | | WKPGSSGP | | |
| 51 | fibrinogen | 806 | PLIK- | 1334 | MRME- | 1738 | RDRQHLPLIK | 2243 | MKPVPDLVPGNF | 2648 | LERPGGNEIT |
| | alpha | | MKPV | | LERP | | | | KSQLQKVPPEWK | | |
| | chain | | | | | | | | ALTDMPQMRME | | |
| 52 | fibrinogen | 807 | GEFV- | 1335 | PSRG- | 1739 | FFSPMLGEFV | 2244 | SETESRGSESGI | 2649 | KSSSYSKQFT |
| | alpha | | SETE | | KSSS | | | | FTNTKESSSHHP | | |
| | chain | | | | | | | | GIAEFPSRG | | |
| 53 | fibrinogen | 808 | AWTA- | 1336 | PRVV- | 1740 | LSVVGTAWTA | 2245 | DSGEGDFLAEGG | 2650 | ERHQSACKDS |
| | alpha | | DSGE | | ERHQ | | | | GVRGPRVV | | |
| | chain | | | | | | | | | | |
| 54 | fibrinogen | 809 | KNNK- | 1337 | SANN- | 1741 | SLFEYQKNNK | 2246 | DSHSLTTNIMEI | 2651 | RDNTYNRVSE |
| | alpha | | DSHS | | RDNT | | | | LRGDFSSANN | | |
| | chain | | | | | | | | | | |
| 55 | fibrinogen | 810 | TAWT- | 1338 | RVVE- | 1742 | VLSVVGTAWT | 2247 | ADSGEGDFLAEG | 2652 | RHQSACKDSD |
| | alpha | | ADSG | | RHQS | | | | GGVRGPRVVE | | |
| | chain | | | | | | | | | | |
| 56 | fibrinogen | 811 | PLIK- | 1339 | KALT- | 1743 | RDRQHLPLIK | 2248 | MKPVPDLVPGNF | 2653 | DMPQMRMELE |
| | alpha | | MKPV | | DMPQ | | | | KSQLQKVPPEWK | | |
| | chain | | | | | | | | ALT | | |
| 57 | fibrinogen | 812 | GQWH- | 1340 | WGTF- | 1744 | SVSGSTGQWH | 2249 | SESGSFRPDSPG | 2654 | EEVSGNVSPG |
| | alpha | | SESG | | EEVS | | | | SGNARPNNPDWG | | |
| | chain | | | | | | | | TF | | |
| 58 | fibrinogen | 813 | KMKP- | 1341 | PGNF- | 1745 | QHLPLIKMKP | 2250 | VPDLVPGNF | 2655 | KSQLQKVPPE |
| | alpha | | VPDL | | KSQL | | | | | | |
| | chain | | | | | | | | | | |
| 59 | fibrinogen | 814 | MRME- | 1342 | SPRN- | 1746 | LTDMPQMRME | 2251 | LERPGGNEITRG | 2656 | PSSAGSWNSG |
| | alpha | | LERP | | PSSA | | | | GSTSYGTGSETE | | |
| | chain | | | | | | | | SPRN | | |
| 60 | fibrinogen | 815 | KPVP- | 1343 | PGNF- | 1747 | LPLIKMKPVP | 2252 | DLVPGNF | 2657 | KSQLQKVPPE |
| | alpha | | DLVP | | KSQL | | | | | | |
| | chain | | | | | | | | | | |
| 61 | fibrinogen | 816 | PLIK- | 1344 | PQMR- | 1748 | RDRQHLPLIK | 2253 | MKPVPDLVPGNF | 2658 | MELERPGGNE |
| | alpha | | MKPV | | MELE | | | | KSQLQKVPPEWK | | |
| | chain | | | | | | | | ALTDMPQMR | | |
| 62 | fibrinogen | 817 | TAWT- | 1345 | ERHQ- | 1749 | VLSVVGTAWT | 2254 | ADSGEGDFLAEG | 2659 | SACKDSDWPF |
| | alpha | | ADSG | | SACK | | | | GGVRGPRVVERH | | |
| | chain | | | | | | | | Q | | |
| 63 | fibrinogen | 818 | GNFK- | 1346 | KALT- | 1750 | VPDLVPGNFK | 2255 | SQLQKVPPEWKA | 2660 | DMPQMRMELE |
| | alpha | | SQLQ | | DMPQ | | | | LT | | |
| | chain | | | | | | | | | | |
| 64 | fibrinogen | 819 | RGKS- | 1347 | TFES- | 1751 | IAEFPSRGKS | 2256 | SSYSKQFTSSTS | 2661 | KSYKMADEAG |
| | alpha | | SSYS | | KSYK | | | | YNRGDSTFES | | |
| | chain | | | | | | | | | | |
| 65 | fibrinogen | 820 | MLGE- | 1348 | PSRG- | 1752 | PGFFSPMLGE | 2257 | FVSETESRGSES | 2662 | KSSSYSKQFT |
| | alpha | | FVSE | | KSSS | | | | GIFTNTKESSSH | | |
| | chain | | | | | | | | HPGIAEFPSRG | | |
| 66 | fibrinogen | 821 | MRME- | 1349 | GNRN- | 1753 | LTDMPQMRME | 2258 | LERPGGNEITRG | 2663 | PGSSGTGGTA |
| | alpha | | LERP | | PGSS | | | | GSTSYGTGSETE | | |
| | chain | | | | | | | | SPRNPSSAGSWN | | |
| | | | | | | | | | SGSSGPGSTGNR | | |
| | | | | | | | | | N | | |
| 67 | fibrinogen | 822 | SYSK- | 1350 | SKSY- | 1754 | SRGKSSSYSK | 2259 | QFTSSTSYNRGD | 2664 | KMADEAGSEA |
| | alpha | | QFTS | | KMAD | | | | STFESKSY | | |
| | chain | | | | | | | | | | |
| 68 | fibrinogen | 823 | ESSV- | 1351 | WGTF- | 1755 | AGHWTSESSV | 2260 | SGSTGQWHSESG | 2665 | EEVSGNVSPG |
| | alpha | | SGST | | EEVS | | | | SFRPDSPGSGNA | | |
| | chain | | | | | | | | RPNNPDWGTF | | |
| 69 | fibrinogen | 824 | DGFR- | 1352 | PSRG- | 1756 | SGIGTLDGFR | 2261 | HRHPDEAAFFDT | 2666 | KSSSYSKQFT |
| | alpha | | HRHP | | KSSS | | | | ASTGKTFPGFFS | | |
| | chain | | | | | | | | PMLGEFVSETES | | |
| | | | | | | | | | RGSESGIFTNTK | | |
| | | | | | | | | | ESSSHHPGIAEF | | |
| | | | | | | | | | PSRG | | |
| 70 | fibrinogen | 825 | PLIK- | 1353 | MELE- | 1757 | RDRQHLPLIK | 2262 | MKPVPDLVPGNF | 2667 | RPGGNEITRG |
| | alpha | | MKPV | | RPGG | | | | KSQLQKVPPEWK | | |
| | chain | | | | | | | | ALTDMPQMRMEL | | |
| | | | | | | | | | E | | |
| 71 | fibrinogen | 826 | WGTF- | 1354 | LVTS- | 1758 | RPNNPDWGTF | 2263 | EEVSGNVSPGTR | 2668 | KGDKELRTGK |
| | alpha | | EEVS | | KGDK | | | | REYHTEKLVTS | | |
| | chain | | | | | | | | | | |
| 72 | fibrinogen | 827 | MRME- | 1355 | GSWN- | 1759 | LTDMPQMRME | 2264 | LERPGGNEITRG | 2669 | SGSSGPGSTG |
| | alpha | | LERP | | SGSS | | | | GSTSYGTGSETE | | |
| | chain | | | | | | | | SPRNPSSAGSWN | | |
| 73 | fibrinogen | 828 | EAAF- | 1356 | TFPG- | 1760 | RHRHPDEAAF | 2265 | FDTASTGKTFPG | 2670 | FFSPMLGEFV |
| | alpha | | FDTA | | FFSP | | | | | | |
| | chain | | | | | | | | | | |
| 74 | fibrinogen | 829 | SRGK- | 1357 | RGHA- | 1761 | GIAEFPSRGK | 2266 | SSSYSKQFTSST | 2671 | KSRPVRDCDD |
| | alpha | | SSSY | | KSRP | | | | SYNRGDSTFESK | | |
| | chain | | | | | | | | SYKMADEAGSEA | | |
| | | | | | | | | | DHEGTHSTKRGH | | |
| | | | | | | | | | A | | |
| 75 | fibrinogen | 830 | PLIK- | 1358 | PGNF- | 1762 | RDRQHLPLIK | 2267 | MKPVPDLVPGNF | 2672 | KSQLQKVPPE |
| | alpha | | MKPV | | KSQL | | | | | | |
| | chain | | | | | | | | | | |
| 76 | alpha-1- | 831 | DPQG- | 1359 | LAHQ- | 1763 | PVSLAEDPQG | 2268 | DAAQKTDTSHHD | 2673 | SNSTNIFFSP |
| | anti- | | DAAQ | | SNST | | | | QDHPTFNKITPN | | |
| | trypsin | | | | | | | | LAEFAFSLYRQL | | |
| | | | | | | | | | AHQ | | |
| 77 | alpha-1- | 832 | FVFL- | | N/A | 1764 | VKFNKPFVFL | | N/A | 2674 | MIEQNTKSPL |
| | anti- | | MIEQ | | (end | | | | | | FMGKVVNPTQ |
| | trypsin | | | | of | | | | | | K |
| | | | | | pro- | | | | | | |
| | | | | | tein) | | | | | | |
| 78 | alpha-1- | 833 | MFLE- | | N/A | 1765 | TEAAGAMFLE | | N/A | 2675 | AIPMSIPPEV |
| | anti- | | AIPM | | (end | | | | | | KFNKPFVFLM |
| | trypsin | | | | of | | | | | | IEQNTKSPLF |
| | | | | | pro- | | | | | | MGKVVNPTQK |
| | | | | | tein) | | | | | | |
| 79 | alpha-1- | 834 | IPMS- | | N/A | 1766 | AMFLEAIPMS | | N/A | 2676 | IPPEVKFNKP |
| | anti- | | IPPE | | (end | | | | | | FVFLMIEQNT |
| | trypsin | | | | of | | | | | | KSPLFMGKV |
| | | | | | pro- | | | | | | VNPTQK |
| | | | | | tein} | | | | | | |
| 80 | alpha-1- | 835 | GTEA- | | N/A | 1767 | LTIDEKGTEA | | N/A | 2677 | AGAMFLEAIP |
| | anti- | | AGAM | | (end | | | | | | MSIPPEVKFN |
| | trypsin | | | | of | | | | | | KPFVFLMIEQ |
| | | | | | pro- | | | | | | NTKSPLFMGK |
| | | | | | tein) | | | | | | VVNPTQK |
| 81 | alpha-1- | 836 | AIPM- | | N/A | 1768 | GAMFLEAIPM | | N/A | 2678 | SIPPEVKFNK |
| | anti- | | SIPP | | (end | | | | | | PFVFLMIEQN |
| | trypsin | | | | of | | | | | | TKSPLFMGKV |
| | | | | | pro- | | | | | | VNPTQK |
| | | | | | tein) | | | | | | |
| 82 | alpha-1- | 837 | PFVF- | | N/A | 1769 | EVKENKPFVF | | N/A | 2679 | LMIEQNTKSP |
| | anti- | | LMIE | | (end | | | | | | LFMGKVVNPT |
| | trypsin | | | | of | | | | | | QK |
| | | | | | pro- | | | | | | |
| | | | | | tein) | | | | | | |
| 83 | alpha-1- | 838 | PEVK- | | N/A | 1770 | IPMSIPPEVK | | N/A | 2680 | FNKPFVFLMI |
| | anti- | | FNKP | | (end | | | | | | EQNTKSPLFM |
| | trypsin | | | | of | | | | | | GKVVNPTQK |
| | | | | | pro- | | | | | | |
| | | | | | tein) | | | | | | |
| 84 | alpha-1- | 839 | AMFL- | | N/A | 1771 | GTEAAGAMFL | | N/A | 2681 | EAIPMSIPPE |
| | anti- | | EAIP | | (end | | | | | | VKFNKPFVFL |
| | trypsin | | | | of | | | | | | MIEQNTKSPL |
| | | | | | pro- | | | | | | FMGKVVNPTQ |
| | | | | | tein) | | | | | | K |
| 85 | alpha-1- | 840 | VSLA- | 1360 | LAHQ- | 1772 | LCCLVPVSLA | 2269 | EDPQGDAAQKTD | 2682 | SNSTNIFFSP |
| | anti- | | EDPQ | | SNST | | | | TSHHDQDHPTFN | | |
| | trypsin | | | | | | | | KITPNLAEFAFS | | |
| | | | | | | | | | LYRQLAHQ | | |
| 86 | alpha-1- | 841 | VFLM- | | N/A | 1773 | KFNKPFVFLM | | N/A | 2683 | IEQNTKSPLF |
| | anti- | | IEQN | | (end | | | | | | MGKVVNPTQK |
| | trypsin | | | | of | | | | | | |
| | | | | | pro- | | | | | | |
| | | | | | tein) | | | | | | |
| 87 | alpha-1- | 842 | AAGA- | | N/A | 1774 | DEKGTEAAGA | | N/A | 2684 | MFLEAIPMSI |
| | anti- | | MFLE | | (end | | | | | | PPEVKFNKPF |
| | trypsin | | | | of | | | | | | VFLMIEQNTK |
| | | | | | pro- | | | | | | SPLFMGKVVN |
| | | | | | tein) | | | | | | PTQK |
| 88 | alpha-1- | 843 | EAIP- | | N/A | 1775 | AGAMFLEAIP | | N/A | 2685 | MSIPPEVKFN |
| | anti- | | MSIP | | (end | | | | | | KPFVFLMIEQ |
| | trypsin | | | | of | | | | | | NTKSPLFMGK |
| | | | | | pro- | | | | | | VVNPTQK |
| | | | | | tein) | | | | | | |
| 89 | alpha-1- | 844 | AGAM- | | N/A | 1776 | EKGTEAAGAM | | N/A | 2686 | FLEAIPMSIP |
| | anti- | | FLEA | | (end | | | | | | PEVKFNKPFV |
| | trypsin | | | | of | | | | | | FLMIEQNTKS |
| | | | | | pro- | | | | | | PLFMGKVVNP |
| | | | | | tein) | | | | | | TQK |
| 90 | alpha-1- | 845 | DPQG- | 1361 | AHQS- | 1777 | PVSLAEDPQG | 2270 | DAAQKTDTSHHD | 2687 | NSTNIFFSPV |
| | anti- | | DAAQ | | NSTN | | | | QDHPTFNKITPN | | |
| | trypsin | | | | | | | | LAEFAFSLYRQL | | |
| | | | | | | | | | AHQS | | |
| 91 | alpha-1- | 846 | DEKG- | | N/A | 1778 | KAVLTIDEKG | | N/A | 2688 | TEAAGAMFLE |
| | anti- | | TEAA | | (end | | | | | | AIPMSIPPEV |
| | trypsin | | | | of | | | | | | KFNKPFVFLM |
| | | | | | pro- | | | | | | IEQNTKSPLF |
| | | | | | tein) | | | | | | MGKVVNPTQK |
| 92 | alpha-1- | 847 | EKGT- | | N/A | 1779 | AVLTIDEKGT | | N/A | 2689 | EAAGAMFLEA |
| | anti- | | EAAG | | (end | | | | | | IPMSIPPEVK |
| | trypsin | | | | of | | | | | | FNKPFVFLMI |
| | | | | | pro- | | | | | | EQNTKSPLFM |
| | | | | | tein) | | | | | | GKVVNPTQK |
| 93 | alpha-1- | 848 | AMFL- | | N/A | 1780 | GTEAAGAMFL | | N/A | 2690 | EAIPMSIPPE |
| | anti- | | EAIP | | (end | | | | | | VKFNKPFVFL |
| | trypsin | | | | of | | | | | | MIEQNTKSPL |
| | | | | | pro- | | | | | | FMGKVVNPTQ |
| | | | | | tein) | | | | | | K |
| 94 | alpha-1- | 849 | VSLA- | 1362 | AHQS- | 1781 | LCCLVPVSLA | 2271 | EDPQGDAAQKTD | 2691 | NSTNIFFSPV |
| | anti- | | EDPQ | | NSTN | | | | TSHHDQDHPTFN | | |
| | trypsin | | | | | | | | KITPNLAEFAFS | | |
| | | | | | | | | | LYRQLAHQS | | |
| 95 | alpha-1- | 850 | VSLA- | 1363 | QLAH- | 1782 | LCCLVPVSLA | 2272 | EDPQGDAAQKTD | 2692 | QSNSTNIFFS |
| | anti- | | EDPQ | | QSNS | | | | TSHHDQDHPTFN | | |
| | trypsin | | | | | | | | KITPNLAEFAFS | | |
| | | | | | | | | | LYRQLAH | | |
| 96 | alpha-1- | 851 | KGTE- | | N/A | 1783 | VLTIDEKGTE | | N/A | 2693 | AAGAMFLEAI |
| | anti- | | AAGA | | (end | | | | | | PMSIPPEVKF |
| | trypsin | | | | of | | | | | | NKPFVFLMIE |
| | | | | | pro- | | | | | | QNTKSPLFMG |
| | | | | | tein) | | | | | | KVVNPTQK |
| 97 | alpha-1- | 852 | DPQG- | 1364 | QLAH- | 1784 | PVSLAEDPQG | 2273 | DAAQKTDTSHHD | 2694 | QSNSTNIFFS |
| | anti- | | DAAQ | | QSNS | | | | QDHPTFNKITPN | | |
| | trypsin | | | | | | | | LAEFAFSLYRQL | | |
| | | | | | | | | | AH | | |
| 98 | alpha-1- | 853 | TEAA- | | N/A | 1785 | TIDEKGTEAA | | N/A | 2695 | GAMFLEAIPM |
| | anti- | | GAMF | | (end | | | | | | SIPPEVKFNK |
| | trypsin | | | | of | | | | | | PFVFLMIEQN |
| | | | | | pro- | | | | | | TKSPLFMGKV |
| | | | | | tein) | | | | | | VNPTQK |
| 99 | alpha-1- | 854 | IDEK- | | N/A | 1786 | HKAVLTIDEK | | N/A | 2696 | GTEAAGAMFL |
| | anti- | | GTEA | | (end | | | | | | EAIPMSIPPE |
| | trypsin | | | | of | | | | | | VKFNKPFVFL |
| | | | | | pro- | | | | | | MIEQNTKSPL |
| | | | | | tein) | | | | | | FMGKVVNPTQ |
| | | | | | | | | | | | K |
| 100 | alpha-1- | 855 | EAAG- | | N/A | 1787 | IDEKGTEAAG | | N/A | 2697 | AMFLEAIPMS |
| | anti- | | AMFL | | (end | | | | | | IPPEVKFNKP |
| | trypsin | | | | of | | | | | | FVFLMIEQNT |
| | | | | | pro- | | | | | | KSPLFMGKVV |
| | | | | | tein) | | | | | | NPTQK |
| 101 | alpha-1- | 856 | VSLA- | 1365 | RQLA- | 1788 | LCCLVPVSLA | 2274 | EDPQGDAAQKTD | 2698 | HQSNSTNIFF |
| | anti- | | EDPQ | | HQSN | | | | TSHHDQDHPTFN | | |
| | trypsin | | | | | | | | KITPNLAEFAFS | | |
| | | | | | | | | | LYRQLA | | |
| 102 | alpha-1- | 857 | LAED- | 1366 | LAHQ- | 1789 | CLVPVSLAED | 2275 | PQGDAAQKTDTS | 2699 | SNSTNIFFSP |
| | anti- | | PQGD | | SNST | | | | HHDQDHPTFNKI | | |
| | trypsin | | | | | | | | TPNLAEFAFSLY | | |
| | | | | | | | | | RQLAHQ | | |
| 103 | Complement | 858 | LSLQ- | 1367 | QVVK- | 1790 | ASSFFTLSLQ | 2276 | KPRLLLFSPSVV | 2700 | GSVFLRNPSR |
| | C4-B OR | | KPRL | | GSVF | | | | HLGVPLSVGVQL | | |
| | Complement | | | | | | | | QDVPRGQVVK | | |
| | C4-A | | | | | | | | | | |
| 104 | Complement | 859 | LSLQ- | 1368 | VPRG- | 1791 | ASSFFTLSLQ | 2277 | KPRLLLFSPSVV | 2701 | QVVKGSVFLR |
| | C4-B OR | | KPRL | | QVVK | | | | HLGVPLSVGVQL | | |
| | Complement | | | | | | | | QDVPRG | | |
| | C4-A | | | | | | | | | | |
| 105 | Complement | 860 | LSLQ- | 1369 | PSRN- | 1792 | ASSFFTLSLQ | 2278 | KPRLLLFSPSVV | 2702 | NVPCSPKVDF |
| | C4-B OR | | KPRL | | NVPC | | | | HLGVPLSVGVQL | | |
| | Complement | | | | | | | | QDVPRGQVVKGS | | |
| | C4-A | | | | | | | | VFLRNPSRN | | |
| 106 | Complement | 861 | LSLQ- | 1370 | SRNN- | 1793 | ASSFFTLSLQ | 2279 | KPRLLLFSPSVV | 2703 | VPCSPKVDFT |
| | C4-B OR | | KPRL | | VPCS | | | | HLGVPLSVGVQL | | |
| | Complement | | | | | | | | QDVPRGQVVKGS | | |
| | C4-A | | | | | | | | VFLRNPSRNN | | |
| 107 | Complement | 862 | LSLQ- | 1371 | VPLS- | 1794 | ASSFFTLSLQ | 2280 | KPRLLLFSPSVV | 2704 | VGVQLQDVPR |
| | C4-B OR | | KPRL | | VGVQ | | | | HLGVPLS | | |
| | Complement | | | | | | | | | | |
| | C4-A | | | | | | | | | | |
| 108 | Complement | 863 | LSLQ- | 1372 | GSVF- | 1795 | ASSFFTLSLQ | 2281 | KPRLLLFSPSVV | 2705 | LRNPSRNNVP |
| | C4-B OR | | KPRL | | LRNP | | | | HLGVPLSVGVQL | | |
| | Complement | | | | | | | | QDVPRGQVVKGS | | |
| | C4-A | | | | | | | | VF | | |
| 109 | Complement | 864 | LSLQ- | 1373 | VQLQ- | 1796 | ASSFFTLSLQ | 2282 | KPRLLLFSPSVV | 2706 | DVPRGQVVKG |
| | C4-B OR | | KPRL | | DVPR | | | | HLGVPLSVGVQL | | |
| | Complement | | | | | | | | Q | | |
| | C4-A | | | | | | | | | | |
| 110 | Complement | 865 | LSLQ- | 1374 | VGVQ- | 1797 | ASSFFTLSLQ | 2283 | KPRLLLFSPSVV | 2707 | LQDVPRGQVV |
| | C4-B OR | | KPRL | | LQDV | | | | HLGVPLSVGVQ | | |
| | Complement | | | | | | | | | | |
| | C4-A | | | | | | | | | | |
| 111 | Complement | 866 | STGR- | 1375 | NRQI- | 1798 | LNVTLSSTGR | 2284 | NGFKSHALQLNN | 2708 | RGLEEELQFS |
| | C4-B OR | | NGFK | | RGLE | | | | RQI | | |
| | Complement | | | | | | | | | | |
| | C4-A | | | | | | | | | | |
| 112 | Complement | 867 | LSLQ- | 1376 | VHLG- | 1799 | ASSFFTLSLQ | 2285 | KPRLLLFSPSVV | 2709 | VPLSVGVQLQ |
| | C4-B OR | | KPRL | | VPLS | | | | HLG | | |
| | Complement | | | | | | | | | | |
| | C4-A | | | | | | | | | | |
| 113 | Complement | 868 | STGR- | 1377 | RQIR- | 1800 | LNVTLSSTGR | 2286 | NGFKSHALQLNN | 2710 | GLEEELQFSL |
| | C4-B OR | | NGFK | | GLEE | | | | RQIR | | |
| | Complement | | | | | | | | | | |
| | C4-A | | | | | | | | | | |
| 114 | Complement | 869 | LPAK- | 1378 | GRRN- | 1801 | DYEYDELPAK | 2287 | DDPDAPLQPVTP | 2711 | RRRREAPKVV |
| | C4-B OR | | DDPD | | RRRR | | | | LQLFEGRRN | | |
| | Complement | | | | | | | | | | |
| | C4-A | | | | | | | | | | |
| 115 | Complement | 870 | LSLQ- | 1379 | SVVH- | 1802 | ASSFFTLSLQ | 2288 | KPRLLLFSPSVV | 2712 | LGVPLSVGVQ |
| | C4-B OR | | KPRL | | LGVP | | | | H | | |
| | Complement | | | | | | | | | | |
| | C4-A | | | | | | | | | | |
| 116 | Complement | 871 | RQIR- | 1380 | INVK- | 1803 | ALQLNNRQIR | 2289 | GLEEELQFSLGS | 2713 | VGGNSKGTLK |
| | C4-B OR | | GLEE | | VGGN | | | | KINVK | | |
| | Complement | | | | | | | | | | |
| | C4-A | | | | | | | | | | |
| 117 | Complement | 872 | PAKD- | 1381 | PVTP- | 1804 | YEYDELPAKD | 2290 | DPDAPLQPVTP | 2714 | LQLFEGRRNR |
| | C4-B OR | | DPDA | | LQLF | | | | | | |
| | Complement | | | | | | | | | | |
| | C4-A | | | | | | | | | | |
| 118 | Complement | 873 | LPAK- | 1382 | PVTP- | 1805 | DYEYDELPAK | 2291 | DDPDAPLQPVTP | 2715 | LQLFEGRRNR |
| | C4-B OR | | DDPD | | LQLF | | | | | | |
| | Complement | | | | | | | | | | |
| | C4-A | | | | | | | | | | |
| 119 | Complement | 874 | TGRN- | 1383 | NRQI- | 1806 | NVTLSSTGRN | 2292 | GFKSHALQLNNR | 2716 | RGLEEELQFS |
| | C4-B OR | | GFKS | | RGLE | | | | QI | | |
| | Complement | | | | | | | | | | |
| | C4-A | | | | | | | | | | |
| 120 | Complement | 875 | LPAK- | 1384 | TPLQ- | 1807 | DYEYDELPAK | 2293 | DDPDAPLQPVTP | 2717 | LFEGRRNRRR |
| | C4-B OR | | DDPD | | LFEG | | | | LQ | | |
| | Complement | | | | | | | | | | |
| | C4-A | | | | | | | | | | |
| 121 | Complement | 876 | LSLQ- | 1385 | VFLR- | 1808 | ASSFFTLSLQ | 2294 | KPRLLLFSPSVV | 2718 | NPSRNNVPCS |
| | C4-B OR | | KPRL | | NPSR | | | | HLGVPLSVGVQL | | |
| | Complement | | | | | | | | QDVPRGQVVKGS | | |
| | C4-A | | | | | | | | VFLR | | |
| 122 | Complement | 877 | LPAK- | 1386 | PLQL- | 1809 | DYEYDELPAK | 2295 | DDPDAPLQPVTP | 2719 | FEGRRNRRRR |
| | C4-B OR | | DDPD | | FEGR | | | | LQL | | |
| | Complement | | | | | | | | | | |
| | C4-A | | | | | | | | | | |
| 123 | Complement | 878 | RQIR- | 1387 | LGSK- | 1810 | ALQLNNRQIR | 2296 | GLEEELQFSLGS | 2720 | INVKVGGNSK |
| | C4-B OR | | GLEE | | INVK | | | | K | | |
| | Complement | | | | | | | | | | |
| | C4-A | | | | | | | | | | |
| 124 | Complement | 879 | HRGR- | 1388 | VRVT- | 1811 | ELNPLDHRGR | 2297 | TLEIPGNSDPNM | 2721 | ASDPLDTLGS |
| | C4-B OR | | TLEI | | ASDP | | | | IPDGDFNSYVRV | | |
| | Complement | | | | | | | | T | | |
| | C4-A | | | | | | | | | | |
| 125 | Complement | 880 | PRLL- | 1389 | VGVQ- | 1812 | TLSLQKPRLL | 2298 | LFSPSVVHLGVP | 2722 | LQDVPRGQVV |
| | C4-B OR | | LFSP | | LQDV | | | | LSVGVQ | | |
| | Complement | | | | | | | | | | |
| | C4-A | | | | | | | | | | |
| 126 | Complement | 881 | SELQ- | 1390 | ARLT- | 1813 | IIPQTISELQ | 2299 | LSVSAGSPHPAI | 2723 | VAAPPSGGPG |
| | C4-B OR | | LSVS | | VAAP | | | | ARLT | | |
| | Complement | | | | | | | | | | |
| | C4-A | | | | | | | | | | |
| 127 | Complement | 882 | ARLT- | 1391 | PRVG- | 1814 | SPHPAIARLT | 2300 | VAAPPSGGPGFL | 2724 | DTLNLNLRAV |
| | C4-B OR | | VAAP | | DTLN | | | | SIERPDSRPPRV | | |
| | Complement | | | | | | | | G | | |
| | C4-A | | | | | | | | | | |
| 128 | Complement | 883 | PRLL- | 1392 | VPLS- | 1815 | TLSLQKPRLL | 2301 | LFSPSVVHLGVP | 2725 | VGVQLQDVPR |
| | C4-B OR | | LFSP | | VGVQ | | | | LS | | |
| | Complement | | | | | | | | | | |
| | C4-A | | | | | | | | | | |
| 129 | Complement | 884 | AKDD- | 1393 | GRRN- | 1816 | EYDELPAKDD | 2302 | PDAPLQPVTPLQ | 2726 | RRRREAPKVV |
| | C4-B OR | | PDAP | | RRRR | | | | LFEGRRN | | |
| | Complement | | | | | | | | | | |
| | C4-A | | | | | | | | | | |
| 130 | Complement | 885 | PAKD- | 1394 | GRRN- | 1817 | YEYDELPAKD | 2303 | DPDAPLQPVTPL | 2727 | RRRREAPKVV |
| | C4-B OR | | DPDA | | RRRR | | | | QLFEGRRN | | |
| | Complement | | | | | | | | | | |
| | C4-A | | | | | | | | | | |
| 131 | Complement | 886 | RLLL- | 1395 | VPLS- | 1818 | LSLQKPRLLL | 2304 | FSPSVVHLGVPL | 2728 | VGVQLQDVPR |
| | C4-B OR | | FSPS | | VGVQ | | | | S | | |
| | Complement | | | | | | | | | | |
| | C4-A | | | | | | | | | | |
| 132 | Complement | 887 | DYEY- | 1396 | GRRN- | 1819 | ANEDYEDYEY | 2305 | DELPAKDDPDAP | 2729 | RRRREAPKVV |
| | C4-B OR | | DELP | | RRRR | | | | LQPVTPLQLFEG | | |
| | Complement | | | | | | | | RRN | | |
| | C4-A | | | | | | | | | | |
| 133 | Complement | 888 | PRLL- | 1397 | QVVK- | 1820 | TLSLQKPRLL | 2306 | LFSPSVVHLGVP | 2730 | GSVFLRNPSR |
| | C4-B OR | | LFSP | | GSVF | | | | LSVGVQLQDVPR | | |
| | Complement | | | | | | | | GQVVK | | |
| | C4-A | | | | | | | | | | |
| 134 | Complement | 889 | PRLL- | 1398 | VHLG- | 1821 | TLSLQKPRLL | 2307 | LFSPSVVHLG | 2731 | VPLSVGVQLQ |
| | C4-B OR | | LFSP | | VPLS | | | | | | |
| | Complement | | | | | | | | | | |
| | C4-A | | | | | | | | | | |
| 135 | Complement | 890 | LTVA- | 1399 | PRVG- | 1822 | HPAIARLTVA | 2308 | APPSGGPGFLSI | 2732 | DTLNLNLRAV |
| | C4-B OR | | APPS | | DTLN | | | | ERPDSRPPRVG | | |
| | Complement | | | | | | | | | | |
| | C4-A | | | | | | | | | | |
| 136 | fibrinogen | 891 | DKKR- | 1400 | GGGY- | 1823 | RGHRPLDKKR | 2309 | EEAPSLRPAPPP | 2733 | RARPAKAAAT |
| | beta | | EEAP | | RARP | | | | ISGGGY | | |
| | chain | | | | | | | | | | |
| 137 | fibrinogen | 892 | KKRE- | 1401 | GGGY- | 1824 | GHRPLDKKRE | 2310 | EAPSLRPAPPPI | 2734 | RARPAKAAAT |
| | beta | | EAPS | | RARP | | | | SGGGY | | |
| | chain | | | | | | | | | | |
| 138 | fibrinogen | 893 | LDKK- | 1402 | GGGY- | 1825 | ARGHRPLDKK | 2311 | REEAPSLRPAPP | 2735 | RARPAKAAAT |
| | beta | | REEA | | RARP | | | | PISGGGY | | |
| | chain | | | | | | | | | | |
| 139 | fibrinogen | 894 | LVKS- | 1403 | GGGY- | 1826 | LLLCVFLVKS | 2312 | QGVNDNEEGFFS | 2736 | RARPAKAAAT |
| | beta | | QGVN | | RARP | | | | ARGHRPLDKKRE | | |
| | chain | | | | | | | | EAPSLRPAPPPI | | |
| | | | | | | | | | SGGGY | | |
| 140 | fibrinogen | 895 | LVKS- | 1404 | FSAR- | 1827 | LLLCVFLVKS | 2313 | QGVNDNEEGFFS | 2737 | GHRPLDKKRE |
| | beta | | QGVN | | GHRP | | | | AR | | |
| | chain | | | | | | | | | | |
| 141 | fibrinogen | 896 | LVKS- | 1405 | ARGH- | 1828 | LLLCVFLVKS | 2314 | QGVNDNEEGFFS | 2738 | RPLDKKREEA |
| | beta | | QGVN | | RPLD | | | | ARGH | | |
| | chain | | | | | | | | | | |
| 142 | fibrinogen | 897 | LVKS- | 1406 | FFSA- | 1829 | LLLCVFLVKS | 2315 | QGVNDNEEGFFS | 2739 | RGHRPLDKKR |
| | beta | | QGVN | | RGHR | | | | A | | |
| | chain | | | | | | | | | | |
| 143 | fibrinogen | 898 | LVKS- | 1407 | KKRE- | 1830 | LLLCVFLVKS | 2316 | QGVNDNEEGFFS | 2740 | EAPSLRPAPP |
| | beta | | QGVN | | EAPS | | | | ARGHRPLDKKRE | | |
| | chain | | | | | | | | | | |
| 144 | fibrinogen | 899 | PLDK- | 1408 | GGGY- | 1831 | SARGHRPLDK | 2317 | KREEAPSLRPAP | 2741 | RARPAKAAAT |
| | beta | | KREE | | RARP | | | | PPISGGGY | | |
| | chain | | | | | | | | | | |
| 145 | fibrinogen | 900 | LVKS- | 1409 | GGYR- | 1832 | LLLCVFLVKS | 2318 | QGVNDNEEGFFS | 2742 | ARPAKAAAT |
| | beta | | QGVN | | ARPA | | | | ARGHRPLDKKRE | | |
| | chain | | | | | | | | EAPSLRPAPPPI | | |
| | | | | | | | | | SGGGYR | | |
| 146 | fibrinogen | 901 | LVKS- | 1410 | RPLD- | 1833 | LLLCVFLVKS | 2319 | QGVNDNEEGFFS | 2743 | KKREEAPSLR |
| | beta | | QGVN | | KKRE | | | | ARGHRPLD | | |
| | chain | | | | | | | | | | |
| 147 | fibrinogen | 902 | HRPL- | 1411 | GGGY- | 1834 | FFSARGHRPL | 2320 | DKKREEAPSLRP | 2744 | RARPAKAAAT |
| | beta | | DKKR | | RARP | | | | APPPISGGGY | | |
| | chain | | | | | | | | | | |
| 148 | fibrinogen | 903 | LVKS- | 1412 | DKKR- | 1835 | LLLCVFLVKS | 2321 | QGVNDNEEGFFS | 2745 | EEAPSLRPAP |
| | beta | | QGVN | | EEAP | | | | ARGHRPLDKKR | | |
| | chain | | | | | | | | | | |
| 149 | fibrinogen | 904 | SQGV- | 1413 | FFSA- | 1836 | CVFLVKSQGV | 2322 | NDNEEGFFSA | 2746 | RGHRPLDKKR |
| | beta | | NDNE | | RGHR | | | | | | |
| | chain | | | | | | | | | | |
| 150 | fibrinogen | 905 | LVKS- | 1414 | RPAK- | 1837 | LLLCVFLVKS | 2323 | QGVNDNEEGFFS | 2747 | AAATQKKVER |
| | beta | | QGVN | | AAAT | | | | ARGHRPLDKKRE | | |
| | chain | | | | | | | | EAPSLRPAPPPI | | |
| | | | | | | | | | SGGGYRARPAK | | |
| 151 | fibrinogen | 906 | LVKS- | 1415 | GFFS- | 1838 | LLLCVFLVKS | 2324 | QGVNDNEEGFFS | 2748 | ARGHRPLDKK |
| | beta | | QGVN | | ARGH | | | | | | |
| | chain | | | | | | | | | | |
| 152 | fibrinogen | 907 | KSQG- | 1416 | RPLD- | 1839 | LCVFLVKSQG | 2325 | VNDNEEGFFSAR | 2749 | KKREEAPSLR |
| | beta | | VNDN | | KKRE | | | | GHRPLD | | |
| | chain | | | | | | | | | | |
| 153 | fibrinogen | 908 | KSQG- | 1417 | GGYR- | 1840 | LCVFLVKSQG | 2326 | VNDNEEGFFSAR | 2750 | ARPAKAAATQ |
| | beta | | VNDN | | ARPA | | | | GHRPLDKKREEA | | |
| | chain | | | | | | | | PSLRPAPPPISG | | |
| | | | | | | | | | GGYR | | |
| 154 | fibrinogen | 909 | KSQG- | 1418 | PLDK- | 1841 | LCVFLVKSQG | 2327 | VNDNEEGFFSAR | 2751 | KREEAPSLRP |
| | beta | | VNDN | | KREE | | | | GHRPLDK | | |
| | chain | | | | | | | | | | |
| 155 | fibrinogen | 910 | LVKS- | 1419 | APSL- | 1842 | LLLCVFLVKS | 2328 | QGVNDNEEGFFS | 2752 | RPAPPPISGG |
| | beta | | QGVN | | RPAP | | | | ARGHRPLDKKRE | | |
| | chain | | | | | | | | EAPSL | | |
| 156 | fibrinogen | 911 | LVKS- | 1420 | EAPS- | 1843 | LLLCVFLVKS | 2329 | QGVNDNEEGFFS | 2753 | LRPAPPPISG |
| | beta | | QGVN | | LRPA | | | | ARGHRPLDKKRE | | |
| | chain | | | | | | | | EAPS | | |
| 157 | fibrinogen | 912 | LVKS- | 1421 | LDKK- | 1844 | LLLCVFLVKS | 2330 | QGVNDNEEGFFS | 2754 | REEAPSLRPA |
| | beta | | QGVN | | REEA | | | | ARGHRPLDKK | | |
| | chain | | | | | | | | | | |
| 158 | fibrinogen | 913 | KSQG- | 1422 | FFSA- | 1845 | LCVFLVKSQG | 2331 | VNDNEEGFFSA | 2755 | RGHRPLDKKR |
| | beta | | VNDN | | RGHR | | | | | | |
| | chain | | | | | | | | | | |
| 159 | fibrinogen | 914 | PLDK- | 1423 | GGYR- | 1846 | SARGHRPLDK | 2332 | KREEAPSLRPAP | 2756 | ARPAKAAATQ |
| | beta | | KREE | | ARPA | | | | PPISGGGYR | | |
| | chain | | | | | | | | | | |
| 160 | fibrinogen | 915 | FSAR- | 1424 | GGGY- | 1847 | DNEEGFFSAR | 2333 | GHRPLDKKREEA | 2757 | RARPAKAAAT |
| | beta | | GHRP | | RARP | | | | PSLRPAPPPISG | | |
| | chain | | | | | | | | GGY | | |
| 161 | fibrinogen | 916 | SQGV- | 1425 | RPLD- | 1848 | CVFLVKSQGV | 2334 | NDNEEGFFSARG | 2758 | KKREEAPSLR |
| | beta | | NDNE | | KKRE | | | | HRPLD | | |
| | chain | | | | | | | | | | |
| 162 | fibrinogen | 917 | LVKS- | 1426 | RARP- | 1849 | LLLCVFLVKS | 2335 | QGVNDNEEGFFS | 2759 | AKAAATQKKV |
| | beta | | QGVN | | AKAA | | | | ARGHRPLDKKRE | | |
| | chain | | | | | | | | EAPSLRPAPPPI | | |
| | | | | | | | | | SGGGYRARP | | |
| 163 | fibrinogen | 918 | LVKS- | 1427 | AKAA- | 1850 | LLLCVFLVKS | 2336 | QGVNDNEEGFFS | 2760 | ATQKKVERKA |
| | beta | | QGVN | | ATQK | | | | ARGHRPLDKKRE | | |
| | chain | | | | | | | | EAPSLRPAPPPI | | |
| | | | | | | | | | SGGGYRARPAKA | | |
| | | | | | | | | | A | | |
| 164 | fibrinogen | 919 | MSMK- | | N/A | 1851 | WYSMRKMSMK | | N/A | 2761 | IRPFFPQQ |
| | beta | | IRPF | | (end | | | | | | |
| | chain | | | | of | | | | | | |
| | | | | | pro- | | | | | | |
| | | | | | tein) | | | | | | |
| 165 | fibrinogen | 920 | QGVN- | 1428 | FFSA- | 1852 | VFLVKSQGVN | 2337 | DNEEGFFSA | 2762 | RGHRPLDKKR |
| | beta | | DNEE | | RGHR | | | | | | |
| | chain | | | | | | | | | | |
| 166 | fibrinogen | 921 | LVKS- | 1429 | PPIS- | 1853 | LLLCVFLVKS | 2338 | QGVNDNEEGFFS | 2763 | GGGYRARPAK |
| | beta | | QGVN | | GGGY | | | | ARGHRPLDKKRE | | |
| | chain | | | | | | | | EAPSLRPAPPPI | | |
| | | | | | | | | | S | | |
| 167 | fibrinogen | 922 | LVKS- | 1430 | KREE- | 1854 | LLLCVFLVKS | 2339 | QGVNDNEEGFFS | 2764 | APSLRPAPPP |
| | beta | | QGVN | | APSL | | | | ARGHRPLDKKRE | | |
| | chain | | | | | | | | E | | |
| 168 | fibrinogen | 923 | LVKS- | 1431 | ARPA- | 1855 | LLLCVFLVKS | 2340 | QGVNDNEEGFFS | 2765 | KAAATQKKVE |
| | beta | | QGVN | | KAAA | | | | ARGHRPLDKKRE | | |
| | chain | | | | | | | | EAPSLRPAPPPI | | |
| | | | | | | | | | SGGGYRARPA | | |
| 169 | serum | 924 | NIQR- | | N/A | 1856 | ISDARENIQR | | N/A | 2766 | FFGHGAEDSL |
| | amyloid | | FFGH | | (end | | | | | | ADQAANEWGR |
| | A-1 | | | | of | | | | | | SGKDPNHFRP |
| | protein | | | | pro- | | | | | | AGLPEKY |
| | | | | | tein) | | | | | | |
| 170 | serum | 925 | AAEA- | | N/A | 1857 | GPGGVWAAEA | | N/A | 2767 | ISDARENIQR |
| | amyloid | | ISDA | | (end | | | | | | FFGHGAEDSL |
| | A-1 | | | | of | | | | | | ADQAANEWGR |
| | protein | | | | pro- | | | | | | SGKDPNHFRP |
| | | | | | tein) | | | | | | AGLPEKY |
| 171 | serum | 926 | GAED- | | N/A | 1858 | QRFFGHGAED | | N/A | 2768 | SLADQAANEW |
| | amyloid | | SLAD | | (end | | | | | | GRSGKDPNHF |
| | A-1 | | | | of | | | | | | RPAGLPEKY |
| | protein | | | | pro- | | | | | | |
| | | | | | tein) | | | | | | |
| 172 | serum | 927 | IQRF- | | N/A | 1859 | SDARENIQRF | | N/A | 2769 | FGHGAEDSLA |
| | amyloid | | FGHG | | (end | | | | | | DQAANEWGRS |
| | A-1 | | | | of | | | | | | GKDPNHFRPA |
| | protein | | | | pro- | | | | | | GLPEKY |
| | | | | | tein) | | | | | | |
| 173 | serum | 928 | GVSS- | 1432 | EAFD- | 1860 | FCSLVLGVSS | 2341 | RSFFSFLGEAFD | 2770 | GARDMWRAYS |
| | amyloid | | RSFF | | GARD | | | | | | |
| | A-1 | | | | | | | | | | |
| | protein | | | | | | | | | | |
| 174 | serum | 929 | SGKD- | | N/A | 1861 | ANEWGRSGKD | | N/A | 2771 | PNHFRPAGLP |
| | amyloid | | PNHF | | (end | | | | | | EKY |
| | A-1 | | | | of | | | | | | |
| | protein | | | | pro- | | | | | | |
| | | | | | tein) | | | | | | |
| 175 | serum | 930 | GVWA- | | N/A | 1862 | AKRGPGGVWA | | N/A | 2772 | AEAISDAREN |
| | amyloid | | AEAI | | (end | | | | | | IQRFFGHGAE |
| | A-1 | | | | of | | | | | | DSLADQAANE |
| | protein | | | | pro- | | | | | | WGRSGKDPNH |
| | | | | | tein) | | | | | | FRPAGLPEKY |
| 176 | serum | 931 | ADQA- | | N/A | 1863 | GAEDSLADQA | | N/A | 2773 | ANEWGRSGKD |
| | amyloid | | ANEW | | (end | | | | | | PNHFRPAGLP |
| | A-1 | | | | of | | | | | | EKY |
| | protein | | | | pro- | | | | | | |
| | | | | | tein) | | | | | | |
| 177 | serum | 932 | GEAF- | 1433 | KRGP- | 1864 | SFFSFLGEAF | 2342 | DGARDMWRAYSD | 2774 | GGVWAAEAIS |
| | amyloid | | DGAR | | GGVW | | | | MREANYIGSDKY | | |
| | A-1 | | | | | | | | FHARGNYDAAKR | | |
| | protein | | | | | | | | GP | | |
| 178 | serum | 933 | AISD- | | N/A | 1865 | GVWAAEAISD | | N/A | 2775 | ARENIQRFFG |
| | amyloid | | AREN | | (end | | | | | | HGAEDSLADQ |
| | A-1 | | | | of | | | | | | AANEWGRSGK |
| | protein | | | | pro- | | | | | | DPNHFRPAGL |
| | | | | | tein) | | | | | | PEKY |
| 179 | serum | 934 | VSSR- | 935 | EAFD- | 1866 | CSLVLSVSSR | 2343 | SFFSFLGEAFD | 2776 | GARDMWRAYS |
| | amyloid | | SFFS | | GARD | | | | | | |
| | A-1 pro- | | | | | | | | | | |
| | tein OR | | | | | | | | | | |
| | serum | | | | | | | | | | |
| | amyloid | | | | | | | | | | |
| | A-2 | | | | | | | | | | |
| | protein | | | | | | | | | | |
| 180 | serum | 936 | DSLA- | | N/A | 1867 | FGHGAEDSLA | | N/A | 2777 | DQAANEWGRS |
| | amyloid | | DQAA | | (end | | | | | | GKDPNHFRPA |
| | A-1 | | | | of | | | | | | GLPEKY |
| | protein | | | | pro- | | | | | | |
| | | | | | tein) | | | | | | |
| 181 | serum | 937 | SFFS- | 1434 | KRGP- | 1868 | LSVSSRSFFS | 2344 | FLGEAFDGARDM | 2778 | GGVWAAEVIS |
| | amyloid | | FLGE | | GGVW | | | | WRAYSDMREANY | | |
| | A-1 | | | | | | | | IGSDKYFHARGN | | |
| | protein | | | | | | | | YDAAKRGP | | |
| 182 | serum | 938 | SLAD- | | N/A | 1869 | GHGAEDSLAD | | N/A | 2779 | QAANEWGRSG |
| | amyloid | | QAAN | | (end | | | | | | KDPNHFRPAG |
| | A-1 | | | | of | | | | | | LPEKY |
| | protein | | | | pro- | | | | | | |
| | | | | | tein) | | | | | | |
| 183 | serum | 939 | ENIQ- | | N/A | 1870 | AISDARENIQ | | N/A | 2780 | RFFGHGAEDS |
| | amyloid | | RFFG | | (end | | | | | | LADQAANEWG |
| | A-1 | | | | of | | | | | | RSGKDPNHFR |
| | protein | | | | pro- | | | | | | PAGLPEKY |
| | | | | | tein) | | | | | | |
| 184 | serum | 940 | NIQR- | 1435 | LPEK-Y | 1871 | ISDARENIQR | 2345 | FFGHGAEDSLAD | | N/A |
| | amyloid | | FFGH | | | | | | QAANEWGRSGKD | | |
| | A-1 | | | | | | | | PNHFRPAGLPEK | | |
| | protein | | | | | | | | | | |
| 185 | serum | 941 | FFGH- | | N/A | 1872 | RENIQRFFGH | | N/A | 2781 | GAEDSLADQA |
| | amyloid | | GAED | | (end | | | | | | ANEWGRSGKD |
| | A-1 | | | | of | | | | | | PNHFRPAGLP |
| | protein | | | | pro- | | | | | | EKY |
| | | | | | tein) | | | | | | |
| 186 | serum | 942 | RSGK- | | N/A | 1873 | AANEWGRSGK | | N/A | 2782 | DPNHFRPAGL |
| | amyloid | | DPNH | | (end | | | | | | PEKY |
| | A-1 | | | | of | | | | | | |
| | protein | | | | pro- | | | | | | |
| | | | | | tein) | | | | | | |
| 187 | serum | 943 | RFFG- | | N/A | 1874 | ARENIQRFFG | | N/A | 2783 | HGAEDSLADQ |
| | amyloid | | HGAE | | (end | | | | | | AANEWGRSGK |
| | A-1 | | | | of | | | | | | DPNHFRPAGL |
| | protein | | | | pro- | | | | | | PEKY |
| | | | | | tein) | | | | | | |
| 188 | serum | 944 | EWGR- | | N/A | 1875 | ADQAANEWGR | | N/A | 2784 | SGKDPNHFRP |
| | amyloid | | SGKD | | (end | | | | | | AGLPEKY |
| | A-1 | | | | of | | | | | | |
| | protein | | | | pro- | | | | | | |
| | | | | | tein) | | | | | | |
| 189 | serum | 945 | DQAA- | | N/A | 1876 | AEDSLADQAA | | N/A | 2785 | NEWGRSGKDP |
| | amyloid | | NEWG | | (end | | | | | | NHFRPAGLPE |
| | A-1 | | | | of | | | | | | KY |
| | protein | | | | pro- | | | | | | |
| | | | | | tein) | | | | | | |
| 190 | serum | 946 | EAIS- | | N/A | 1877 | GGVWAAEAIS | | N/A | 2786 | DARENIQRFF |
| | amyloid | | DARE | | (end | | | | | | GHGAEDSLAD |
| | A-1 | | | | of | | | | | | QAANEWGRSG |
| | protein | | | | pro- | | | | | | KDPNHFRPAG |
| | | | | | tein) | | | | | | LPEKY |
| 191 | serum | 947 | RSFF- | 1436 | KRGP- | 1878 | VLSVSSRSFF | 2346 | SFLGEAFDGARD | 2787 | GGVWAAEAIS |
| | amyloid | | SFLG | | GGVW | | | | MWRAYSDMREAN | | |
| | A-1 | | | | | | | | YIGSDKYFHARG | | |
| | protein | | | | | | | | NYDAAKRGP | | |
| 192 | serum | 948 | DSLA- | 1437 | FRPA- | 1879 | FGHGAEDSLA | 2347 | DQAANEWGRSGK | 2788 | GLPEKY |
| | amyloid | | DQAA | | GLPE | | | | DPNHFRPA | | |
| | A-1 | | | | | | | | | | |
| | protein | | | | | | | | | | |
| 193 | serum | 949 | AISD- | 1438 | GAED- | 1880 | GVWAAEAISD | 2348 | ARENIQRFFGHG | 2789 | SLADQAANEW |
| | amyloid | | AREN | | SLAD | | | | AED | | |
| | A-1 | | | | | | | | | | |
| | protein | | | | | | | | | | |
| 194 | serum | 950 | EDSL- | | N/A | 1881 | FFGHGAEDSL | | N/A | 2790 | ADQAANEWGR |
| | amyloid | | ADQA | | (end | | | | | | SGKDPNHFRP |
| | A-1 | | | | of | | | | | | AGLPEKY |
| | protein | | | | pro- | | | | | | |
| | | | | | tein) | | | | | | |
| 195 | serum | 951 | AEAI- | | N/A | 1882 | PGGVWAAEAI | | N/A | 2791 | SDARENIQRF |
| | amyloid | | SDAR | | (end | | | | | | FGHGAEDSLA |
| | A-1 | | | | of | | | | | | DQAANEWGRS |
| | protein | | | | pro- | | | | | | GKDPNHFRPA |
| | | | | | tein) | | | | | | GLPEKY |
| 196 | serum | 952 | FSFL- | 1439 | KRGP- | 1883 | VSSRSFFSFL | 2349 | GEAFDGARDMWR | 2792 | GGVWAAEAIS |
| | amyloid | | GEAF | | GGVW | | | | AYSDMREANYIG | | |
| | A-1 | | | | | | | | SDKYFHARGNYD | | |
| | protein | | | | | | | | AAKRGP | | |
| 197 | serum | 953 | FHAR- | | N/A | 1884 | IGSDKYFHAR | | N/A | 2793 | GNYDAAKRGP |
| | amyloid | | GNYD | | (end | | | | | | GGVWAAEAIS |
| | A-1 | | | | of | | | | | | DARENIQRFF |
| | protein | | | | pro- | | | | | | GHGAEDSLAD |
| | | | | | tein) | | | | | | QAANEWGRSG |
| | | | | | | | | | | | KDPNHFRPA |
| | | | | | | | | | | | GLPEKY |
| 198 | serum | 954 | GVSS- | 1440 | FSFL- | 1885 | FCSLVLGVSS | 2350 | RSFFSFL | 2794 | GEAFDGARDM |
| | amyloid | | RSFF | | GEAF | | | | | | |
| | A-1 pro- | | | | | | | | | | |
| | tein OR | | | | | | | | | | |
| | serum | | | | | | | | | | |
| | amyloid | | | | | | | | | | |
| | A-2 | | | | | | | | | | |
| | protein | | | | | | | | | | |
| 199 | serum | 955 | KRGP- | | N/A | 1886 | GNYDAAKRGP | | N/A | 2795 | GGVWAAEAIS |
| | amyloid | | GGVW | | (end | | | | | | DARENIQRFF |
| | A-1 | | | | of | | | | | | GHGAEDSLAD |
| | protein | | | | pro | | | | | | QAANEWGRSG |
| | | | | | tein) | | | | | | KDPNHFRPAG |
| | | | | | | | | | | | LPEKY |
| 200 | trans- | 956 | FTAN- | | N/A | 1887 | EHAEVVFTAN | | N/A | 2796 | DSGPRRYTIA |
| | thyretin | | DSGP | | (end | | | | | | ALLSPYSYST |
| | | | | | of | | | | | | TAVVTNPKE |
| | | | | | pro- | | | | | | |
| | | | | | tein) | | | | | | |
| 201 | trans- | 957 | ANDS- | | N/A | 1888 | AEVVFTANDS | | N/A | 2797 | GPRRYTIAAL |
| | thyretin | | GPRR | | (end | | | | | | LSPYSYSTTA |
| | | | | | of | | | | | | VVTNPKE |
| | | | | | pro- | | | | | | |
| | | | | | tein) | | | | | | |
| 202 | trans- | 958 | ANDS- | 1441 | TNPK-E | 1889 | AEVVFTANDS | 2351 | GPRRYTIAALLS | | N/A |
| | thyretin | | GPRR | | | | | | PYSYSTTAVVTN | | |
| | | | | | | | | | PK | | |
| 203 | trans- | 959 | TAND- | | N/A | 1890 | HAEVVFTAND | | N/A | 2798 | SGPRRYTIAA |
| | thyretin | | SGPR | | (end | | | | | | LLSPYSYSTT |
| | | | | | of | | | | | | AVVTNPKE |
| | | | | | pro- | | | | | | |
| | | | | | tein) | | | | | | |
| 204 | trans- | 960 | NDSG- | | N/A | 1891 | EVVFTANDSG | | N/A | 2799 | PRRYTIAALL |
| | thyretin | | PRRY | | (end | | | | | | SPYSYSTTAV |
| | | | | | of | | | | | | VTNPKE |
| | | | | | pro- | | | | | | |
| | | | | | tein) | | | | | | |
| 205 | trans- | 961 | AALL- | | N/A | 1892 | PRRYTIAALL | | N/A | 2800 | SPYSYSTTAV |
| | thyretin | | SPYS | | (end | | | | | | VTNPKE |
| | | | | | of | | | | | | |
| | | | | | pro- | | | | | | |
| | | | | | tein) | | | | | | |
| 206 | trans- | 962 | YTIA- | | N/A | 1893 | DSGPRRYTIA | | N/A | 2801 | ALLSPYSYST |
| | thyretin | | ALLS | | (end | | | | | | TAVVTNPKE |
| | | | | | of | | | | | | |
| | | | | | pro- | | | | | | |
| | | | | | tein) | | | | | | |
| 207 | trans- | 963 | RRYT- | | N/A | 1894 | ANDSGPRRYT | | N/A | 2802 | IAALLSPYSY |
| | thyretin | | IAAL | | (end | | | | | | STTAVVTNPK |
| | | | | | of | | | | | | E |
| | | | | | pro- | | | | | | |
| | | | | | tein) | | | | | | |
| 208 | trans- | 964 | EVVF- | | N/A | 1895 | PFHEHAEVVF | | N/A | 2803 | TANDSGPRRY |
| | thyretin | | TAND | | (end | | | | | | TIAALL |
| | | | | | of | | | | | | SPYSYSTTAV |
| | | | | | pro- | | | | | | VTNPKE |
| | | | | | tein) | | | | | | |
| 209 | trans- | 965 | KALG- | | N/A | 1896 | DTKSYWKALG | | N/A | 2804 | ISPFHEHAEV |
| | thyretin | | ISPF | | (end | | | | | | VFTANDSGPR |
| | | | | | of | | | | | | RYTIAALLSP |
| | | | | | pro- | | | | | | YSYSTTAVVT |
| | | | | | tein) | | | | | | NPKE |
| 210 | alpha-2- | 966 | PVSA- | 1442 | TALK- | 1897 | PCSVFSPVSA | 2352 | MEPLGRQLTSGP | 2805 | SPPGVCSRDP |
| | anti- | | MEPL | | SPPG | | | | NQEQVSPLTLLK | | |
| | plasmin | | | | | | | | LGNQEPGGQTAL | | |
| | | | | | | | | | K | | |
| 211 | alpha-2- | 967 | PVSA- | 1443 | TLLK- | 1898 | PCSVFSPVSA | 2353 | MEPLGRQLTSGP | 2806 | LGNQEPGGQT |
| | anti- | | MEPL | | LGNQ | | | | NQEQVSPLTLLK | | |
| | plasmin | | | | | | | | | | |
| 212 | alpha-2- | 968 | TSGP- | 1444 | TALK- | 1899 | PLGRQLTSGP | 2354 | NQEQVSPLTLLK | 2807 | SPPGVCSRDP |
| | anti- | | NQEQ | | SPPG | | | | LGNQEPGGQTAL | | |
| | plasmin | | | | | | | | K | | |
| 213 | alpha-2- | 969 | PDLK- | | N/A | 1900 | GDKLFGPDLK | | N/A | 2808 | LVPPMEEDYP |
| | anti- | | LVPP | | (end | | | | | | QFGSPK |
| | plasmin | | | | of | | | | | | |
| | | | | | pro- | | | | | | |
| | | | | | tein) | | | | | | |
| 214 | alpha-2- | 970 | RQLT- | 1445 | TLLK- | 1901 | AMEPLGRQLT | 2355 | SGPNQEQVSPLT | 2809 | LGNQEPGGQT |
| | anti- | | SGPN | | LGNQ | | | | LLK | | |
| | plasmin | | | | | | | | | | |
| 215 | alpha-2- | 971 | PVSA- | 1446 | TSGP- | 1902 | PCSVFSPVSA | 2356 | MEPLGRQLTSGP | 2810 | NQEQVSPLTL |
| | anti- | | MEPL | | NQEQ | | | | | | |
| | plasmin | | | | | | | | | | |
| 216 | alpha-2- | 972 | VSAM- | 1447 | TSGP- | 1903 | CSVFSPVSAM | 2357 | EPLGRQLTSGP | 2811 | NQEQVSPLTL |
| | anti- | | EPLG | | NQEQ | | | | | | |
| | plasmin | | | | | | | | | | |
| 217 | alpha-2- | 973 | TSGP- | 1448 | TLLK- | 1904 | PLGRQLTSGP | 2358 | NQEQVSPLTLLK | 2812 | LGNQEPGGQT |
| | anti- | | NQEQ | | LGNQ | | | | | | |
| | plasmin | | | | | | | | | | |
| 218 | alpha-2- | 974 | TSGP- | 1449 | LKLG- | 1905 | PLGRQLTSGP | 2359 | NQEQVSPLTLLK | 2813 | NQEPGGQTAL |
| | anti- | | NQEQ | | NQEP | | | | LG | | |
| | plasmin | | | | | | | | | | |
| 219 | alpha-2- | 975 | AMSR- | 1450 | KEQQ- | 1906 | AAATSIAMSR | 2360 | MSLSSFSVNRPF | 2814 | DSPGNKDFLQ |
| | anti- | | MSLS | | DSPG | | | | LFFIFEDTTGLP | | |
| | plasmin | | | | | | | | LFVGSVRNPNPS | | |
| | | | | | | | | | APRELKEQQ | | |
| 220 | alpha-2- | 976 | PVSA- | 1451 | PLTL- | 1907 | PCSVFSPVSA | 2361 | MEPLGRQLTSGP | 2815 | LKLGNQEPGG |
| | anti- | | MEPL | | LKLG | | | | NQEQVSPLTL | | |
| | plasmin | | | | | | | | | | |
| 221 | apolipo- | 977 | LLPV- | | N/A | 1908 | EDLRQGLLPV | | N/A | 2816 | LESFKVSFLS |
| | protein | | LESF | | (end | | | | | | ALEEYTKKLN |
| | A-I | | | | of | | | | | | TQ |
| | | | | | pro- | | | | | | |
| | | | | | tein) | | | | | | |
| 222 | apolipo- | 978 | LPVL- | | N/A | 1909 | DLRQGLLPVL | | N/A | 2817 | ESFKVSFLSA |
| | protein | | ESFK | | (end | | | | | | LEEYTKKLNT |
| | A-I | | | | of | | | | | | Q |
| | | | | | pro- | | | | | | |
| | | | | | tein) | | | | | | |
| 223 | apolipo | 979 | RVKD- | 1452 | GKQL- | 1910 | PQSPWDRVKD | 2362 | LATVYVDVLKDS | 2818 | NLKLLDNWDS |
| | protein | | LATV | | NLKL | | | | GRDYVSQFEGSA | | |
| | A-I | | | | | | | | LGKQL | | |
| | Isoform 1 | | | | | | | | | | |
| 224 | apolipo- | 980 | FWQQ- | 1453 | DVLK- | 1911 | GSQARHFWQQ | 2363 | DEPPQSPWDRVK | 2819 | DSGRDYVSQF |
| | protein | | DEPP | | DSGR | | | | DLATVYVDVLK | | |
| | A-I | | | | | | | | | | |
| 225 | apolipo- | 981 | PVLE- | | N/A | 1912 | LRQGLLPVLE | | N/A | 2820 | SFKVSFLSAL |
| | protein | | SFKV | | (end | | | | | | EEYTKKLNTQ |
| | A-I | | | | of | | | | | | |
| | | | | | pro- | | | | | | |
| | | | | | tein) | | | | | | |
| 226 | apolipo- | 982 | ESFK- | | N/A | 1913 | GLLPVLESFK | | N/A | 2821 | VSFLSALEEY |
| | protein | | VSFL | | (end | | | | | | TKKLNTQ |
| | A-I | | | | of | | | | | | |
| | | | | | pro- | | | | | | |
| | | | | | tein) | | | | | | |
| 227 | apolipo- | 983 | EFWD- | 1454 | RQEM- | 1914 | LGPVTQEFWD | 2364 | NLEKETEGLRQE | 2822 | SKDLEEVKAK |
| | protein | | NLEK | | SKDL | | | | M | | |
| | A-I | | | | | | | | | | |
| 228 | apolipo- | 984 | VSFL- | | N/A | 1915 | VLESFKVSFL | | N/A | 2823 | SALEEYTKKL |
| | protein | | SALE | | (end | | | | | | NTQ |
| | A-I | | | | of | | | | | | |
| | | | | | pro- | | | | | | |
| | | | | | tein) | | | | | | |
| 229 | apolipo- | 985 | SFKV- | | N/A | 1916 | LLPVLESFKV | | N/A | 2824 | SFLSALEEYT |
| | protein | | SFLS | | (end | | | | | | KKLNTQ |
| | A-I | | | | of | | | | | | |
| | | | | | pro- | | | | | | |
| | | | | | tein) | | | | | | |
| 230 | apolipo- | 986 | FWQQ- | 1455 | RVKD- | 1917 | GSQARHFWQQ | 2365 | DEPPQSPWDRVK | 2825 | LATVYVDVLK |
| | protein | | DEPP | | LATV | | | | D | | |
| | A-I | | | | | | | | | | |
| 231 | apolipo- | 987 | EPLR- | 1456 | HELQ- | 1918 | LYRQKVEPLR | 2366 | AELQEGARQKLH | 2826 | EKLSPLGEEM |
| | protein | | AELQ | | EKLS | | | | ELQ | | |
| | A-I | | | | | | | | | | |
| 232 | apolipo- | 988 | EPLR- | 1457 | SPLG- | 1919 | LYRQKVEPLR | 2367 | AELQEGARQKLH | 2827 | EEMRDRARAH |
| | protein | | AELQ | | EEMR | | | | ELQEKLSPLG | | |
| | A-I | | | | | | | | | | |
| 233 | apolipo- | 989 | FWQQ- | 1458 | ATVY- | 1920 | GSQARHFWQQ | 2368 | DEPPQSPWDRVK | 2828 | VDVLKDSGRD |
| | protein | | DEPP | | VDVL | | | | DLATVY | | |
| | A-I | | | | | | | | | | |
| 234 | alpha-1- | 990 | LLSA- | | N/A | 1921 | TAVKITLLSA | | N/A | 2829 | LVETRTIVRF |
| | antichy- | | LVET | | (end | | | | | | NRPFLMIIVP |
| | motrypsin | | | | of | | | | | | TDTQNIFFMS |
| | | | | | pro- | | | | | | KVTNPKQA |
| | | | | | tein) | | | | | | |
| 235 | alpha-1- | 991 | ITLL- | | N/A | 1922 | AATAVKITLL | | N/A | 2830 | SALVETRTIV |
| | antichy- | | SALV | | (end | | | | | | RFNRPFLMII |
| | motrypsin | | | | of | | | | | | VPTDTQNIFF |
| | | | | | pro- | | | | | | MSKVTNPKQA |
| | | | | | tein) | | | | | | |
| 236 | alpha-1- | 992 | LSAL- | | N/A | 1923 | AVKITLLSAL | | N/A | 2831 | VETRTIVRFN |
| | antichy- | | VETR | | (end | | | | | | RPFLMIIVPT |
| | motrypsin | | | | of | | | | | | DTQNIFFMSK |
| | | | | | pro- | | | | | | VTNPKQA |
| | | | | | tein) | | | | | | |
| 237 | alpha-1- | 993 | ALVE- | | N/A | 1924 | KITLLSALVE | | N/A | 2832 | TRTIVRFNRP |
| | antichy- | | TRTI | | (end | | | | | | FLMIIVPTDT |
| | motrypsin | | | | of | | | | | | QNIFFMSKVT |
| | | | | | pro- | | | | | | NPKQA |
| | | | | | tein) | | | | | | |
| 238 | alpha-1- | 994 | VPTD- | | N/A | 1925 | PFLMIIVPTD | | N/A | 2833 | TQNIFFMSKV |
| | antichy- | | TQNI | | (end | | | | | | TNPKQA |
| | motrypsin | | | | of | | | | | | |
| | | | | | pro- | | | | | | |
| | | | | | tein) | | | | | | |
| 239 | alpha-1- | 995 | TLLS- | | N/A | 1926 | ATAVKITLLS | | N/A | 2834 | ALVETRTIVR |
| | antichy- | | ALVE | | (end | | | | | | FNRPFLMIIV |
| | motrypsin | | | | of | | | | | | PTDTQNIFFM |
| | | | | | pro- | | | | | | SKVTNPKQA |
| | | | | | tein) | | | | | | |
| 240 | alpha-1- | 996 | LCHP- | 1459 | ENLT- | 1927 | GFCPAVLCHP | 2369 | NSPLDEENLT | 2835 | QENQDRGTHV |
| | antichy- | | NSPL | | QENQ | | | | | | |
| | motrypsin | | | | | | | | | | |
| 241 | alpha-1- | 997 | PFLM- | | N/A | 1928 | IVRFNRPFLM | | N/A | 2836 | IIVPTDTQNI |
| | antichy- | | IIVP | | (end | | | | | | FFMSKVTNPK |
| | motrypsin | | | | of | | | | | | QA |
| | | | | | pro- | | | | | | |
| | | | | | tein) | | | | | | |
| 242 | alpha-1- | 998 | AVLC- | 1460 | LGLA- | 1929 | AAGFCPAVLC | 2370 | HPNSPLDEENLT | 2837 | SANVDFAFSL |
| | antichy- | | HPNS | | SANV | | | | QENQDRGTHVDL | | |
| | motrypsin | | | | | | | | GLA | | |
| 243 | glucagon | 999 | GSWQ- | 1461 | MNED- | 1930 | FVMLVQGSWQ | 2371 | RSLQDTEEKSRS | 2838 | KRHSQGTFTS |
| | | | RSLQ | | KRHS | | | | FSASQADPLSDP | | |
| | | | | | | | | | DQMNED | | |
| 244 | glucagon | 1000 | GSWQ- | 1462 | QMNE- | 1931 | FVMLVQGSWQ | 2372 | RSLQDTEEKSRS | 2839 | DKRHSQGTFT |
| | | | RSLQ | | DKRH | | | | FSASQADPLSDP | | |
| | | | | | | | | | DQMNE | | |
| 245 | glucagon | 1001 | WQRS- | 1463 | MNED- | 1932 | MLVQGSWQRS | 2373 | LQDTEEKSRSFS | 2840 | KRHSQGTFTS |
| | | | LQDT | | KRHS | | | | ASQADPLSDPDQ | | |
| | | | | | | | | | MNED | | |
| 246 | glucagon | 1002 | SWQR- | 1464 | MNED- | 1933 | VMLVQGSWQR | 2374 | SLQDTEEKSRSF | 2841 | KRHSQGTFTS |
| | | | SLQD | | KRHS | | | | SASQADPLSDPD | | |
| | | | | | | | | | QMNED | | |
| 247 | glucagon | 1003 | EDKR- | 1465 | RAQD- | 1934 | DPDQMNEDKR | 2375 | HSQGTFTSDYSK | 2842 | FVQWLMNTKR |
| | | | HSQG | | FVQW | | | | YLDSRRAQD | | |
| 248 | glucagon | 1004 | WQRS- | 1466 | QMNE- | 1935 | MLVQGSWQRS | 2376 | LQDTEEKSRSFS | 2843 | DKRHSQGTFT |
| | | | LQDT | | DKRH | | | | ASQADPLSDPDQ | | |
| | | | | | | | | | MNE | | |
| 249 | glucagon | 1005 | EDKR- | 1467 | LMNT- | 1936 | DPDQMNEDKR | 2377 | HSQGTFTSDYSK | 2844 | KRNRNNIAKR |
| | | | HSQG | | KRNR | | | | YLDSRRAQDFVQ | | |
| | | | | | | | | | WLMNT | | |
| 250 | glucagon | 1006 | QRSL- | 1468 | MNED- | 1937 | LVQGSWQRSL | 2378 | QDTEEKSRSFSA | 2845 | KRHSQGTFTS |
| | | | QDTE | | KRHS | | | | SQADPLSDPDQM | | |
| | | | | | | | | | NED | | |
| 251 | glucagon | 1007 | SWQR- | 1469 | QMNE- | 1938 | VMLVQGSWQR | 2379 | SLQDTEEKSRSF | 2846 | DKRHSQGTFT |
| | | | SLQD | | DKRH | | | | SASQADPLSDPD | | |
| | | | | | | | | | QMNE | | |
| 252 | glucagon | 1008 | RGRR- | 1470 | KITD- | 1939 | AWLVKGRGRR | 2380 | DFPEEVAIVEEL | | N/A |
| | | | DFPE | | RK | | | | GRRHADGSFSDE | | |
| | | | | | | | | | MNTILDNLAARD | | |
| | | | | | | | | | FINWLIQTKITD | | |
| 253 | glucagon | 1009 | IAKR- | 1471 | KGRG- | 1940 | KRNRNNIAKR | 2381 | HDEFERHAEGTF | 2847 | RRDFPEEVAI |
| | | | HDEF | | RRDF | | | | TSDVSSYLEGQA | | |
| | | | | | | | | | AKEFIAWLVKGR | | |
| | | | | | | | | | G | | |
| 254 | hepcidin | 1010 | LTSG- | 1472 | PMFQ- | 1941 | LLLLASLTSG | 2382 | SVFPQQTGQLAE | 2848 | RRRRRDTHFP |
| | | | SVFP | | RRRR | | | | LQPQDRAGARAS | | |
| | | | | | | | | | WMPMFQ | | |
| 255 | hepcidin | 1011 | LTSG- | 1473 | WMPM- | 1942 | LLLLASLTSG | 2383 | SVFPQQTGQLAE | 2849 | FQRRRRRDTH |
| | | | SVFP | | FQRR | | | | LQPQDRAGARAS | | |
| | | | | | | | | | WMPM | | |
| 256 | hepcidin | 1012 | LTSG- | 1474 | SWMP- | 1943 | LLLLASLTSG | 2384 | SVFPQQTGQLAE | 2850 | MFQRRRRRDT |
| | | | SVFP | | MFQR | | | | LQPQDRAGARAS | | |
| | | | | | | | | | WMP | | |
| 257 | hepcidin | 1013 | LTSG- | 1475 | GARA- | 1944 | LLLLASLTSG | 2385 | SVFPQQTGQLAE | 2851 | SWMPMFQRRR |
| | | | SVFP | | SWMP | | | | LQPQDRAGARA | | |
| 258 | hepcidin | 1014 | LTSG- | 1476 | ARAS- | 1945 | LLLLASLTSG | 2386 | SVFPQQTGQLAE | 2852 | WMPMFQRRRR |
| | | | SVFP | | WMPM | | | | LQPQDRAGARAS | | |
| 259 | hepcidin | 1015 | LTSG- | 1477 | RAGA- | 1946 | LLLLASLTSG | 2387 | SVFPQQTGQLAE | 2853 | RASWMPMFQR |
| | | | SVFP | | RASW | | | | LQPQDRAGA | | |
| 260 | hepcidin | 1016 | SVFP- | 1478 | ARAS- | 1947 | ASLTSGSVFP | 2388 | QQTGQLAELQPQ | 2854 | WMPMFQRRRR |
| | | | QQTG | | WMPM | | | | DRAGARAS | | |
| 261 | hepcidin | 1017 | TSGS- | 1479 | ARAS- | 1948 | LLLASLTSGS | 2389 | VFPQQTGQLAEL | 2855 | WMPMFQRRRR |
| | | | VFPQ | | WMPM | | | | QPQDRAGARAS | | |
| 262 | hepcidin | 1018 | SVFP- | 1480 | RAGA- | 1949 | ASLTSGSVFP | 2390 | QQTGQLAELQPQ | 2856 | RASWMPMFQR |
| | | | QQTG | | RASW | | | | DRAGA | | |
| 263 | hepcidin | 1019 | LTSG- | 1481 | QLAE- | 1950 | LLLLASLTSG | 2391 | SVFPQQTGQLAE | 2857 | LQPQDRAGAR |
| | | | SVFP | | LQPQ | | | | | | |
| 264 | hepcidin | 1020 | LTSG- | 1482 | AGAR- | 1951 | LLLLASLTSG | 2392 | SVFPQQTGQLAE | 2858 | ASWMPMFQRR |
| | | | SVFP | | ASWM | | | | LQPQDRAGAR | | |
| 265 | hepcidin | 1021 | LTSG- | 1483 | ELQP- | 1952 | LLLLASLTSG | 2393 | SVFPQQTGQLAE | 2859 | QDRAGARASW |
| | | | SVFP | | QDRA | | | | LQP | | |
| 266 | serum | 1022 | NIQR- | | N/A | 1953 | GPNARENIQR | | N/A | 2860 | LTGRGAEDSL |
| | amyloid | | LTGR | | (end | | | | | | ADQAANKWGR |
| | A-2 | | | | of | | | | | | SGRDPNHFRP |
| | protein | | | | pro- | | | | | | AGLPEKY |
| | | | | | tein) | | | | | | |
| 267 | serum | 1023 | IQRL- | | N/A | 1954 | PNARENIQRL | | N/A | 2861 | TGRGAEDSLA |
| | amyloid | | TGRG | | (end | | | | | | DQAANKWGRS |
| | A-2 | | | | of | | | | | | GRDPNHFRPA |
| | protein | | | | pro- | | | | | | GLPEKY |
| | | | | | tein) | | | | | | |
| 268 | serum | 1024 | RSGR- | | N/A | 1955 | AANKWGRSGR | | N/A | 2862 | DPNHFRPAGL |
| | amyloid | | DPNH | | (end | | | | | | PEKY |
| | A-2 | | | | of | | | | | | |
| | protein | | | | pro- | | | | | | |
| | | | | | tein) | | | | | | |
| 269 | serum | 1025 | QRLT- | | N/A | 1956 | NARENIQRLT | | N/A | 2863 | GRGAEDSLAD |
| | amyloid | | GRGA | | (end | | | | | | QAANKWGRSG |
| | A-2 | | | | of | | | | | | RDPNHFRPAG |
| | protein | | | | pro- | | | | | | LPEKY |
| | | | | | tein) | | | | | | |
| 270 | serum | 1026 | IQRL- | 1484 | DPNH- | 1957 | PNARENIQRL | 2394 | TGRGAEDSLADQ | 2864 | FRPAGLPEKY |
| | amyloid | | TGRG | | FRPA | | | | AANKWGRSGRDP | | |
| | A-2 | | | | | | | | NH | | |
| | protein | | | | | | | | | | |
| 271 | serum | 1027 | NIQR- | 1485 | DPNH- | 1958 | GPNARENIQR | 2395 | LTGRGAEDSLAD | 2865 | FRPAGLPEKY |
| | amyloid | | LTGR | | FRPA | | | | QAANKWGRSGRD | | |
| | A-2 | | | | | | | | PNH | | |
| | protein | | | | | | | | | | |
| 272 | serum | 1028 | GAED- | | N/A | 1959 | QRLTGRGAED | | N/A | 2866 | SLADQAANKW |
| | amyloid | | SLAD | | (end | | | | | | GRSGRDPNHF |
| | A-2 | | | | of | | | | | | RPAGLPEKY |
| | protein | | | | pro- | | | | | | |
| | | | | | tein) | | | | | | |
| 273 | serum | 1029 | RLTG- | | N/A | 1960 | ARENIQRLTG | | N/A | 2867 | RGAEDSLADQ |
| | amyloid | | RGAE | | (end | | | | | | AANKWGRSGR |
| | A-2 | | | | of | | | | | | DPNHFRPAGL |
| | protein | | | | pro- | | | | | | PEKY |
| | | | | | tein) | | | | | | |
| 274 | serum | 1030 | TGRG- | 1486 | DPNH- | 1961 | ENIQRLTGRG | 2396 | AEDSLADQAANK | 2868 | FRPAGLPEKY |
| | amyloid | | AEDS | | FRPA | | | | WGRSGRDPNH | | |
| | A-2 | | | | | | | | | | |
| | protein | | | | | | | | | | |
| 275 | serum | 1031 | AAKR- | 1487 | AEVI- | 1962 | ARGNYDAAKR | 2397 | GPGGAWAAEVI | 2869 | SNARENIQRL |
| | amyloid | | GPGG | | SNAR | | | | | | |
| | A-2 | | | | | | | | | | |
| | protein | | | | | | | | | | |
| 276 | serum | 1032 | GAWA- | | N/A | 1963 | AKRGPGGAWA | | N/A | 2870 | AEVISNAREN |
| | amyloid | | AEVI | | (end | | | | | | IQRLTGRGAE |
| | A-2 | | | | of | | | | | | DSLADQAANK |
| | protein | | | | pro- | | | | | | WGRSGRDPNH |
| | | | | | tein) | | | | | | FRPAGLPEKY |
| 277 | serum | 1033 | DSLA- | | N/A | 1964 | TGRGAEDSLA | | N/A | 2871 | DQAANKWGRS |
| | amyloid | | DQAA | | (end | | | | | | GRDPNHFRPA |
| | A-2 | | | | of | | | | | | GLPEKY |
| | protein | | | | pro- | | | | | | |
| | | | | | tein) | | | | | | |
| 278 | serum | 1034 | AWAA- | | N/A | 1965 | KRGPGGAWAA | | N/A | 2872 | EVISNARENI |
| | amyloid | | EVIS | | (end | | | | | | QRLTGRGAED |
| | A-2 | | | | of | | | | | | SLADQAANKW |
| | protein | | | | pro- | | | | | | GRSGRDPNHF |
| | | | | | tein) | | | | | | RPAGLPEKY |
| 279 | thymosin | 1035 | M- | 1488 | KSKL- | | N/A | 2398 | SDKPDMAEIEKF | 2873 | KKTETQEKNP |
| | beta-4 | | SDKP | | KKTE | | | | DKSKL | | |
| 280 | thymosin | 1036 | M- | 1489 | KKTE- | | N/A | 2399 | SDKPDMAEIEKF | 2874 | TQEKNPLPSK |
| | beta-4 | | SDKP | | TQEK | | | | DKSKLKKTE | | |
| 281 | thymosin | 1037 | KTET- | | N/A | 1966 | DKSKLKKTET | | N/A | 2875 | QEKNPLPSKE |
| | beta-4 | | QEKN | | (end | | | | | | TIEQEKQAGE |
| | | | | | of | | | | | | S |
| | | | | | pro- | | | | | | |
| | | | | | tein) | | | | | | |
| 282 | thymosin | 1038 | M- | | N/A | | N/A | | N/A | 2876 | SDKPDMAEIE |
| | beta-4 | | SDKP | | (end | | | | | | KFDKSKLKKT |
| | | | | | of | | | | | | ETQEKNPLPS |
| | | | | | pro- | | | | | | KETIEQEKQA |
| | | | | | tein) | | | | | | GES |
| 283 | thymosin | 1039 | M- | 1490 | ETQE- | | N/A | 2400 | SDKPDMAEIEKF | 2877 | KNPLPSKETI |
| | beta-4 | | SDKP | | KNPL | | | | DKSKLKKTETQE | | |
| 284 | thymosin | 1040 | KKTE- | | N/A | 1967 | FDKSKLKKTE | | N/A | 2878 | TQEKNPLPSK |
| | beta-4 | | TQEK | | (end | | | | | | ETIEQEKQAG |
| | | | | | of | | | | | | ES |
| | | | | | pro- | | | | | | |
| | | | | | tein) | | | | | | |
| 285 | thymosin | 1041 | ETQE- | | N/A | 1968 | SKLKKTETQE | | N/A | 2879 | KNPLPSKETI |
| | beta-4 | | KNPL | | (end | | | | | | EQEKQAGES |
| | | | | | of | | | | | | |
| | | | | | pro- | | | | | | |
| | | | | | tein) | | | | | | |
| 286 | thymosin | 1042 | KLKK- | | N/A | 1969 | EKFDKSKLKK | | N/A | 2880 | TETQEKNPLP |
| | beta-4 | | TETQ | | (end | | | | | | SKETIEQEKQ |
| | | | | | of | | | | | | AGES |
| | | | | | pro- | | | | | | |
| | | | | | tein) | | | | | | |
| 287 | thymosin | 1043 | TETQ- | | N/A | 1970 | KSKLKKTETQ | | N/A | 2881 | EKNPLPSKET |
| | beta-4 | | EKNP | | (end | | | | | | IEQEKQAGES |
| | | | | | of | | | | | | |
| | | | | | pro- | | | | | | |
| | | | | | tein) | | | | | | |
| 288 | thymosin | 1044 | TQEK- | | N/A | 1971 | KLKKTETQEK | | N/A | 2882 | NPLPSKETIE |
| | beta-4 | | NPLP | | (end | | | | | | QEKQAGES |
| | | | | | of | | | | | | |
| | | | | | pro- | | | | | | |
| | | | | | tein) | | | | | | |
| 289 | hapto- | 1045 | PVQR- | 1491 | MVSH- | 1972 | PKNPANPVQR | 2401 | ILGGHLDAKGSF | 2883 | HNLTTGATLI |
| | globin | | ILGG | | HNLT | | | | PWQAKMVSH | | |
| 290 | hapto- | 1046 | PVQR- | 1492 | VSHH- | 1973 | PKNPANPVQR | 2402 | ILGGHLDAKGSF | 2884 | NLTTGATLIN |
| | globin | | ILGG | | NLTT | | | | PWQAKMVSHH | | |
| 291 | hapto- | 1047 | GVYV- | | N/A | 1974 | CAVAEYGVYV | | N/A | 2885 | KVTSIQDWVQ |
| | globin | | KVTS | | (end | | | | | | KTIAEN |
| | | | | | of | | | | | | |
| | | | | | pro- | | | | | | |
| | | | | | tein) | | | | | | |
| 292 | hapto- | 1048 | PVQR- | 1493 | AKMV- | 1975 | PKNPANPVQR | 2403 | ILGGHLDAKGSF | 2886 | SHHNLTTGAT |
| | globin | | ILGG | | SHHN | | | | PWQAKMV | | |
| 293 | hapto- | 1049 | PVQR- | 1494 | FPWQ- | 1976 | PKNPANPVQR | 2404 | ILGGHLDAKGSF | 2887 | AKMVSHHNLT |
| | globin | | ILGG | | AKMV | | | | PWQ | | |
| 294 | hapto- | 1050 | SALG- | 1495 | LWGQ- | 1977 | MSALG | 2405 | AVIALLLWGQ | 2888 | LFAVDSGNDV |
| | globin | | AVIA | | LFAV | | | | | | |
| 295 | hapto- | 1051 | MSAL- | 1496 | LWGQ- | 1978 | MSAL | 2406 | GAVIALLLWGQ | 2889 | LFAVDSGNDV |
| | globin | | GAVI | | LFAV | | | | | | |
| 296 | hemoglobin | 1052 | HCLL- | | N/A | 1979 | NFKLLSHCLL | | N/A | 2890 | VTLAAHLPAE |
| | subunit | | VTLA | | (end | | | | | | FTPAVHASLD |
| | alpha | | | | of | | | | | | KFLASVSTVL |
| | | | | | pro- | | | | | | TSKYR |
| | | | | | tein) | | | | | | |
| 297 | hemoglobin | 1053 | LLVT- | | N/A | 1980 | KLLSHCLLVT | | N/A | 2891 | LAAHLPAEFT |
| | subunit | | LAAH | | (end | | | | | | PAVHASLDKF |
| | alpha | | | | of | | | | | | LASVSTVLTS |
| | | | | | pro- | | | | | | KYR |
| | | | | | tein) | | | | | | |
| 298 | hemoglobin | 1054 | M- | 1497 | LERM- | | N/A | 2407 | VLSPADKTNVKA | 2892 | FLSFPTTKTY |
| | subunit | | VLSP | | FLSF | | | | AWGKVGAHAGEY | | |
| | alpha | | | | | | | | GAEALERM | | |
| 299 | hemoglobin | 1055 | M- | 1498 | ERMF- | | N/A | 2408 | VLSPADKTNVKA | 2893 | LSFPTTKTYF |
| | subunit | | VLSP | | LSFP | | | | AWGKVGAHAGEY | | |
| | alpha | | | | | | | | GAEALERMF | | |
| 300 | hemoglobin | 1056 | ASLD- | | N/A | 1981 | FTPAVHASLD | | N/A | 2894 | KFLASVSTVL |
| | subunit | | KFLA | | (end | | | | | | TSKYR |
| | alpha | | | | of | | | | | | |
| | | | | | pro- | | | | | | |
| | | | | | tein) | | | | | | |
| 301 | hemoglobin | 1057 | LVTL- | | N/A | 1982 | LLSHCLLVTL | | N/A | 2895 | AAHLPAEFTP |
| | subunit | | AAHL | | (end | | | | | | AVHASLDKFL |
| | alpha | | | | of | | | | | | ASVSTVLTSK |
| | | | | | pro- | | | | | | YR |
| | | | | | tein) | | | | | | |
| 302 | hemoglobin | 1058 | M- | 1499 | FLSF- | | N/A | 2409 | VLSPADKTNVKA | 2896 | PTTKTYFPHF |
| | subunit | | VLSP | | PTTK | | | | AWGKVGAHAGEY | | |
| | alpha | | | | | | | | GAEALERMFLSF | | |
| 303 | hemoglobin | 1059 | M- | 1500 | GKVG- | | N/A | 2410 | VLSPADKTNVKA | 2897 | AHAGEYGAEA |
| | subunit | | VLSP | | AHAG | | | | AWGKVG | | |
| | alpha | | | | | | | | | | |
| 304 | caveolae- | 1060 | QKVR- | | N/A | 1983 | VALEQAQKVR | | N/A | 2898 | YEGSYALTSE |
| | associated | | YEGS | | (end | | | | | | EAERSDGDPV |
| | protein 2 | | | | of | | | | | | QPAVLQVHQT |
| | | | | | pro- | | | | | | S |
| | | | | | tein) | | | | | | |
| 305 | caveolae- | 1061 | M- | 1501 | SDMR- | | N/A | 2411 | GEDAAQAEKFQH | 2899 | QEKPSSPSPM |
| | associated | | GEDA | | QEKP | | | | PGSDMR | | |
| | protein 2 | | | | | | | | | | |
| 306 | caveolae- | 1062 | EGSY- | | N/A | 1984 | AQKVRYEGSY | | N/A | 2900 | ALTSEEAERS |
| | associated | | ALTS | | (end | | | | | | DGDPVQPAVL |
| | protein 2 | | | | of | | | | | | QVHQTS |
| | | | | | pro- | | | | | | |
| | | | | | tein) | | | | | | |
| 307 | caveolae- | 1063 | M- | 1502 | QHPG- | | N/A | 2412 | GEDAAQAEKFQH | 2901 | SDMRQEKPSS |
| | associated | | GEDA | | SDMR | | | | PG | | |
| | protein 2 | | | | | | | | | | |
| 308 | caveolae- | 1064 | M- | 1503 | GSDM- | | N/A | 2413 | GEDAAQAEKFQH | 2902 | RQEKPSSPSP |
| | associated | | GEDA | | RQEK | | | | PGSDM | | |
| | protein 2 | | | | | | | | | | |
| 309 | caveolae- | 1065 | RYEG- | | N/A | 1985 | EQAQKVRYEG | | N/A | 2903 | SYALTSEEAE |
| | associated | | SYAL | | (end | | | | | | RSDGDPVQPA |
| | protein 2 | | | | of | | | | | | VLQVHQTS |
| | | | | | pro- | | | | | | |
| | | | | | tein) | | | | | | |
| 310 | alpha-2- | 1066 | PPLG- | 1504 | HVLL- | 1986 | PDAPPSPPLG | 2414 | APGLPPAGSPPD | 2904 | AAPPGHQLHR |
| | HS-glyco- | | APGL | | AAPP | | | | SHVLL | | |
| | protein | | | | | | | | | | |
| 311 | alpha-2- | 1067 | RKTR- | | N/A | 1987 | GEVSHPRKTR | | N/A | 2905 | TVVQPSVGAA |
| | HS-glyco- | | TVVQ | | (end | | | | | | AGPVVPPCPG |
| | protein | | | | of | | | | | | RIRHFKV |
| | | | | | pro- | | | | | | |
| | | | | | tein) | | | | | | |
| 312 | alpha-2- | 1068 | PPLG- | 1505 | VLLA- | 1988 | PDAPPSPPLG | 2415 | APGLPPAGSPPD | 2906 | APPGHQLHRA |
| | HS-glyco- | | APGL | | APPG | | | | SHVLLA | | |
| | protein | | | | | | | | | | |
| 313 | alpha-2- | 1069 | HVLL- | 1506 | PRKT- | 1989 | GSPPDSHVLL | 2416 | AAPPGHQLHRAH | 2907 | RTVVQPSVGA |
| | HS-glyco- | | AAPP | | RTVV | | | | YDLRHTFMGVVS | | |
| | protein | | | | | | | | LGSPSGEVSHPR | | |
| | | | | | | | | | KT | | |
| 314 | alpha-2- | 1070 | PPLG- | 1507 | PRKT- | 1990 | PDAPPSPPLG | 2417 | APGLPPAGSPPD | 2908 | RTVVQPSVGA |
| | HS-glyco- | | APGL | | RTVV | | | | SHVLLAAPPGHQ | | |
| | protein | | | | | | | | LHRAHYDLRHTF | | |
| | | | | | | | | | MGVVSLGSPSGE | | |
| | | | | | | | | | VSHPRKT | | |
| 315 | alpha-2- | 1071 | VLLA- | 1508 | PRKT- | 1991 | SPPDSHVLLA | 2418 | APPGHQLHRAHY | 2909 | RTVVQPSVGA |
| | HS-glyco- | | APPG | | RTVV | | | | DLRHTFMGVVSL | | |
| | protein | | | | | | | | GSPSGEVSHPRK | | |
| | | | | | | | | | T | | |
| 316 | alpha-2- | 1072 | PPLG- | 1509 | SHVL- | 1992 | PDAPPSPPLG | 2419 | APGLPPAGSPPD | 2910 | LAAPPGHQLH |
| | HS-glyco- | | APGL | | LAAP | | | | SHVL | | |
| | protein | | | | | | | | | | |
| 317 | alpha-2- | 1073 | HVLL- | 1510 | SHPR- | 1993 | GSPPDSHVLL | 2420 | AAPPGHQLHRAH | 2911 | KTRTVVQPSV |
| | HS-glyco- | | AAPP | | KTRT | | | | YDLRHTFMGVVS | | |
| | protein | | | | | | | | LGSPSGEVSHPR | | |
| 318 | chromo- | 1074 | QQKK- | 1511 | KDVM- | 1994 | AKERAHQQKK | 2421 | HSGFEDELSEVL | 2912 | EKREDSKEAE |
| | granin-A | | HSGF | | EKRE | | | | ENQSSQAELKEA | | |
| | | | | | | | | | VEEPSSKDVM | | |
| 319 | chromo- | 1075 | QQKK- | 1512 | DVME- | 1995 | AKERAHQQKK | 2422 | HSGFEDELSEVL | 2913 | KREDSKEAEK |
| | granin-A | | HSGF | | KRED | | | | ENQSSQAELKEA | | |
| | | | | | | | | | VEEPSSKDVME | | |
| 320 | chromo- | 1076 | LQVR- | 1513 | ALRR- | 1996 | LEAGLPLQVR | 2423 | GYPEEKKEEEGS | | N/A |
| | granin-A | | GYPE | | G | | | | ANRRPEDQELES | | |
| | | | | | | | | | LSAIEAELEKVA | | |
| | | | | | | | | | HQLQALRR | | |
| 321 | chromo- | 1077 | QQKK- | 1514 | KDVM- | 1997 | AKERAHQQKK | 2424 | HSGFEDELSEVL | 2914 | EKREDSKEAE |
| | granin-A | | HSGF | | EKRE | | | | ENQSSQAELKEA | | |
| | | | | | | | | | VEEPSSKDVM | | |
| 322 | chromo- | 1078 | AEKR- | 1515 | GPQL- | 1998 | LAKELTAEKR | 2425 | LEGQEEEEDNRD | 2915 | RRGWRPSSRE |
| | granin-A | | LEGQ | | RRGW | | | | SSMKLSFRARAY | | |
| | | | | | | | | | GFRGPGPQL | | |
| 323 | chromo- | 1079 | AEKR- | 1516 | KLSF- | 1999 | LAKELTAEKR | 2426 | LEGQEEEEDNRD | 2916 | RARAYGFRGP |
| | granin-A | | LEGQ | | RARA | | | | SSMKLSF | | |
| 324 | complement | 1080 | LPSR- | 1517 | SLLR- | 2000 | LDVSLQLPSR | 2427 | SSKITHRIHWES | 2917 | SEETKENEGF |
| | C3 | | SSKI | | SEET | | | | ASLLR | | |
| 325 | complement | 1081 | LPSR- | 1518 | ASLL- | 2001 | LDVSLQLPSR | 2428 | SSKITHRIHWES | 2918 | RSEETKENEG |
| | C3 | | SSKI | | RSEE | | | | ASLL | | |
| 326 | complement | 1082 | SSKI- | 1519 | ASLL- | 2002 | LQLPSRSSKI | 2429 | THRIHWESASLL | 2919 | RSEETKENEG |
| | C3 | | THRI | | RSEE | | | | | | |
| 327 | complement | 1083 | SRSS- | 1520 | ASLL- | 2003 | VSLQLPSRSS | 2430 | KITHRIHWESAS | 2920 | RSEETKENEG |
| | C3 | | KITH | | RSEE | | | | LL | | |
| 328 | complement | 1084 | LALG- | 1521 | HDAQ- | 2004 | LLTHLPLALG | 2431 | SPMYSIITPNIL | 2921 | GDVPVTVTVH |
| | C3 | | SPMY | | GDVP | | | | RLESEETMVLEA | | |
| | | | | | | | | | HDAQ | | |
| 329 | complement | 1085 | KITH- | 1522 | ASLL- | 2005 | LPSRSSKITH | 2432 | RIHWESASLL | 2922 | RSEETKENEG |
| | C3 | | RIHW | | RSEE | | | | | | |
| 330 | complement | 1086 | SKIT- | 1523 | ASLL- | 2006 | QLPSRSSKIT | 2433 | HRIHWESASLL | 2923 | RSEETKENEG |
| | C3 | | HRIH | | RSEE | | | | | | |
| 331 | complement | 1087 | THRI - | 1524 | ASLL- | 2007 | SRSSKITHRI | 2434 | HWESASLL | 2924 | RSEETKENEG |
| | C3 | | HWES | | RSEE | | | | | | |
| 332 | complement | 1088 | ITHR- | 1525 | ASLL- | 2008 | PSRSSKITHR | 2435 | IHWESASLL | 2925 | RSEETKENEG |
| | C3 | | IHWE | | RSEE | | | | | | |
| 333 | vitronec- | 1089 | FWGR- | 1526 | PSLA- | 2009 | DIFELLEWGR | 2436 | TSAGTRQPQFIS | 2926 | KKQRFRHRNR |
| | tin | | TSAG | | KKQR | | | | RDWHGVPGQVDA | | |
| | | | | | | | | | AMAGRIYISGMA | | |
| | | | | | | | | | PRPSLA | | |
| 334 | vitronec- | 1090 | TSAG- | 1527 | IYIS- | 2010 | LLFWGRTSAG | 2437 | TRQPQFISRDWH | 2927 | GMAPRPSLAK |
| | tin | | TRQP | | GMAP | | | | GVPGQVDAAMAG | | |
| | | | | | | | | | RIYIS | | |
| 335 | vitronec- | 1091 | LTSD- | 1528 | KPEG- | 2011 | QVGGPSLTSD | 2438 | LQAQSKGNPEQT | 2928 | IDSRPETLHP |
| | tin | | LQAQ | | IDSR | | | | PVLKPEEEAPAP | | |
| | | | | | | | | | EVGASKPEG | | |
| 336 | vitronec- | 1092 | FELL- | 1529 | PSLA- | 2012 | DSWEDIFELL | 2439 | FWGRTSAGTRQP | 2929 | KKQRFRHRNR |
| | tin | | FWGR | | KKQR | | | | QFISRDWHGVPG | | |
| | | | | | | | | | QVDAAMAGRIYI | | |
| | | | | | | | | | SGMAPRPSLA | | |
| 337 | vitronec- | 1093 | TSAG- | 1530 | APRP- | 2013 | LLFWGRTSAG | 2440 | TRQPQFISRDWH | 2930 | SLAKKQRFRH |
| | tin | | TRQP | | SLAK | | | | GVPGQVDAAMAG | | |
| | | | | | | | | | RIYISGMAPRP | | |
| 338 | vitronec- | 1094 | FWGR- | 1531 | PSLA- | 2014 | DIFELLFWGR | 2441 | TSAGTRQPQFIS | 2931 | KKQRFRHRNR |
| | tin | | TSAG | | KKQR | | | | RDWHGVPGQVDA | | |
| | | | | | | | | | AMAGRIYISGMA | | |
| | | | | | | | | | PRPSLA | | |
| 339 | vitronec- | 1095 | FELL- | 1532 | PRPS- | 2015 | DSWEDIFELL | 2442 | FWGRTSAGTRQP | 2932 | LAKKQRFRHR |
| | tin | | FWGR | | LAKK | | | | QFISRDWHGVPG | | |
| | | | | | | | | | QVDAAMAGRIYI | | |
| | | | | | | | | | SGMAPRPS | | |
| 340 | hemopexin | 1096 | QGHN- | 1533 | KLLQ- | 2016 | VDAAFRQGHN | 2443 | SVFLIKGDKVWV | 2933 | DEFPGIPSPL |
| | OR | | SVFL | | DEFP | | | | YPPEKKEKGYPK | | |
| | epididymis | | | | | | | | LLQ | | |
| | secretory | | | | | | | | | | |
| | sperm | | | | | | | | | | |
| | binding | | | | | | | | | | |
| | protein | | | | | | | | | | |
| 341 | hemopexin | 1097 | QGHN- | 1534 | PPEK- | 2017 | VDAAFRQGHN | 2444 | SVFLIKGDKVWV | 2934 | KEKGYPKLLQ |
| | OR | | SVFL | | KEKG | | | | YPPEK | | |
| | epididymis | | | | | | | | | | |
| | secretory | | | | | | | | | | |
| | sperm | | | | | | | | | | |
| | binding | | | | | | | | | | |
| | protein | | | | | | | | | | |
| 342 | hemopexin | 1098 | QGHN- | 1535 | EKKE- | 2018 | VDAAFRQGHN | 2445 | SVFLIKGDKVWV | 2935 | KGYPKLLQDE |
| | OR | | SVFL | | KGYP | | | | YPPEKKE | | |
| | epididymis | | | | | | | | | | |
| | secretory | | | | | | | | | | |
| | sperm | | | | | | | | | | |
| | binding | | | | | | | | | | |
| | protein | | | | | | | | | | |
| 343 | hemopexin | 1099 | RWKN- | 1536 | QGHN- | 2019 | RELISERWKN | 2446 | FPSPVDAAFRQG | 2936 | SVFLIKGDKV |
| | OR | | FPSP | | SVFL | | | | HN | | |
| | epididymis | | | | | | | | | | |
| | secretory | | | | | | | | | | |
| | sperm | | | | | | | | | | |
| | binding | | | | | | | | | | |
| | protein | | | | | | | | | | |
| 344 | hemopexin | 1100 | QGHN- | 1537 | YPPE- | 2020 | VDAAFRQGHN | 2447 | SVFLIKGDKVWV | 2937 | KKEKGYPKLL |
| | OR | | SVFL | | KKEK | | | | YPPE | | |
| | epididymis | | | | | | | | | | |
| | secretory | | | | | | | | | | |
| | sperm | | | | | | | | | | |
| | binding | | | | | | | | | | |
| | protein | | | | | | | | | | |
| 345 | hemopexin | 1101 | DKVW- | 1538 | KLLQ- | 2021 | VFLIKGDKVW | 2448 | VYPPEKKEKGYP | 2938 | DEFPGIPSPL |
| | OR | | VYPP | | DEFP | | | | KLLQ | | |
| | epididymis | | | | | | | | | | |
| | secretory | | | | | | | | | | |
| | sperm | | | | | | | | | | |
| | binding | | | | | | | | | | |
| | protein | | | | | | | | | | |
| 346 | zyxin | 1102 | QTQF- | 1539 | QSQT- | 2022 | PAPAQSQTQF | 2449 | HVQPQPQPKPQV | 2939 | QPVSLANTQP |
| | | | HVQP | | QPVS | | | | QLHVQSQT | | |
| 347 | zyxin | 1103 | QPVS- | 1540 | PVAS- | 2023 | HVQSQTQPVS | 2450 | LANTQPRGPPAS | 2940 | KFSPGAPGGS |
| | | | LANT | | KFSP | | | | SPAPAPKFSPVT | | |
| | | | | | | | | | PKFTPVAS | | |
| 348 | zyxin | 1104 | QTQF- | 1541 | LHVQ- | 2024 | PAPAQSQTQF | 2451 | HVQPQPQPKPQV | 2941 | SQTQPVSLAN |
| | | | HVQP | | SQTQ | | | | QLHVQ | | |
| 349 | zyxin | 1105 | M- | 1542 | APAF- | | N/A | 2452 | AAPRPSPAISVS | 2942 | YAPQKKFGPV |
| | | | AAPR | | YAPQ | | | | VSAPAF | | |
| 350 | zyxin | 1106 | QTQF- | 1543 | QPVS- | 2025 | PAPAQSQTQF | 2453 | HVQPQPQPKPQV | 2943 | LANTQPRGPP |
| | | | HVQP | | LANT | | | | QLHVQSQTQPVS | | |
| 351 | zyxin | 1107 | PKPK- | 1544 | QRAQ- | 2026 | FGPVVAPKPK | 2454 | VNPFRPGDSEPP | 2944 | MGRVGEIPPP |
| | | | VNPF | | MGRV | | | | PAPGAQRAQ | | |
| 352 | apolipo- | 1108 | SARA- | 1545 | TAKD- | 2027 | LLALLASARA | 2455 | SEAEDASLLSFM | 2945 | ALSSVQESQV |
| | protein | | SEAE | | ALSS | | | | QGYMKHATKTAK | | |
| | C-III | | | | | | | | D | | |
| 353 | apolipo- | 1109 | SARA- | 1546 | TKTA- | 2028 | LLALLASARA | 2456 | SEAEDASLLSFM | 2946 | KDALSSVQES |
| | protein | | SEAE | | KDAL | | | | QGYMKHATKTA | | |
| | C-III | | | | | | | | | | |
| 354 | apolipo- | 1110 | FSEF- | | N/A | 2029 | STVKDKFSEF | | N/A | 2947 | WDLDPEVRPT |
| | protein | | WDLD | | (end | | | | | | SAVAA |
| | C-III | | | | of | | | | | | |
| | | | | | pro- | | | | | | |
| | | | | | tein) | | | | | | |
| 355 | apolipo- | 1111 | SARA- | 1547 | AQQA- | 2030 | LLALLASARA | 2457 | SEAEDASLLSFM | 2948 | RGWVTDGFSS |
| | protein | | SEAE | | RGWV | | | | QGYMKHATKTAK | | |
| | C-III | | | | | | | | DALSSVQESQVA | | |
| | | | | | | | | | QQA | | |
| 356 | apolipo- | 1112 | WDLD- | | N/A | 2031 | DKFSEFWDLD | | N/A | 2949 | PEVRPTSAVA |
| | protein | | PEVR | | (end | | | | | | A |
| | | | | | of | | | | | | |
| | | | | | pro- | | | | | | |
| | | | | | tein) | | | | | | |
| 357 | secreto- | 1113 | KRFP- | 1548 | EHIA- | 2032 | KLAPVSKRFP | 2458 | VGPPKNDDTPNR | 2950 | KRAMENM |
| | granin-2 | | VGPP | | KRAM | | | | QYWDEDLLMKVL | | |
| | | | | | | | | | EYLNQEKAEKGR | | |
| | | | | | | | | | EHIA | | |
| 358 | secreto- | 1114 | VSKR- | 1549 | EHIA- | 2033 | TDKLAPVSKR | 2459 | FPVGPPKNDDTP | 2951 | KRAMENM |
| | granin-2 | | FPVG | | KRAM | | | | NRQYWDEDLLMK | | |
| | | | | | | | | | VLEYLNQEKAEK | | |
| | | | | | | | | | GREHIA | | |
| 359 | secreto- | 1115 | KRFP- | 1550 | GREH- | 2034 | KLAPVSKRFP | 2460 | VGPPKNDDTPNR | 2952 | IAKRAMENM |
| | granin-2 | | VGPP | | IAKR | | | | QYWDEDLLMKVL | | |
| | | | | | | | | | EYLNQEKAEKGR | | |
| | | | | | | | | | EH | | |
| 360 | secreto- | 1116 | KRVP- | 1551 | APVS- | 2035 | INSNQVKRVP | 2461 | GQGSSEDDLQEE | 2953 | KRFPVGPPKN |
| | granin-2 | | GQGS | | KRFP | | | | EQIEQAIKEHLN | | |
| | | | | | | | | | QGSSQETDKLAP | | |
| | | | | | | | | | VS | | |
| 361 | secreto- | 1117 | QVKR- | 1552 | APVS- | 2036 | EIINSNQVKR | 2462 | VPGQGSSEDDLQ | 2954 | KRFPVGPPKN |
| | granin-2 | | VPGQ | | KRFP | | | | EEEQIEQAIKEH | | |
| | | | | | | | | | LNQGSSQETDKL | | |
| | | | | | | | | | APVS | | |
| 362 | secreto- | 1118 | PKTP- | 1553 | DGLS- | 2037 | LSKSGYPKTP | 2463 | GRAGTEALPDGL | 2955 | VEDILNLLGM |
| | granin-2 | | GRAG | | VEDI | | | | S | | |
| 363 | secreto- | 1119 | ERKL- | 1554 | PMYE- | 2038 | ETQQWPERKL | 2464 | KHMQFPPMYE | 2956 | ENSRDNPFKR |
| | granin-2 | | KHMQ | | ENSR | | | | | | |
| 364 | angioten- | 1120 | QQLN- | | N/A | 2039 | EPTESTQQLN | | N/A | 2957 | KPEVLEVTLN |
| | sinogen | | KPEV | | (end | | | | | | RPFLFAVYDQ |
| | | | | | of | | | | | | SATALHFLGR |
| | | | | | pro- | | | | | | VANPLSTA |
| | | | | | tein) | | | | | | |
| 365 | angioten- | 1121 | EPTE- | | N/A | 2040 | LEADEREPTE | | N/A | 2958 | STQQLNKPEV |
| | sinogen | | STQQ | | (end | | | | | | LEVTLNRPFL |
| | | | | | of | | | | | | FAVYDQSATA |
| | | | | | pro- | | | | | | LHFLGRVAN |
| | | | | | tein) | | | | | | PLSTA |
| 366 | angioten- | 1122 | TEST- | | N/A | 2041 | ADEREPTEST | | N/A | 2959 | QQLNKPEVLE |
| | sinogen | | QQLN | | (end | | | | | | VTLNRPFLFA |
| | | | | | of | | | | | | VYDQSATALH |
| | | | | | pro- | | | | | | FLGRVANPLS |
| | | | | | tein) | | | | | | TA |
| 367 | c-reactive | 1123 | HAFG- | 1555 | VSLK- | 2042 | VLTSLSHAFG | 2465 | QTDMSRKAFVFP | 2960 | APLTKPLKAF |
| | protein | | QTDM | | APLT | | | | KESDTSYVSLK | | |
| 368 | c-reactive | 1124 | HAFG- | 1556 | APLT- | 2043 | VLTSLSHAFG | 2466 | QTDMSRKAFVFP | 2961 | KPLKAFTVCL |
| | protein | | QTDM | | KPLK | | | | KESDTSYVSLKA | | |
| | | | | | | | | | PLT | | |
| 369 | c-reactive | 1125 | HAFG- | 1557 | SYVS- | 2044 | VLTSLSHAFG | 2467 | QTDMSRKAFVFP | 2962 | LKAPLTKPLK |
| | protein | | QTDM | | LKAP | | | | KESDTSYVS | | |
| 370 | serum | 1126 | VFRR- | 1558 | QYLQ- | 2045 | SAYSRGVFRR | 2468 | DAHKSEVAHRFK | 2963 | QCPFEDHVKL |
| | albumin | | DAHK | | QCPF | | | | DLGEENFKALVL | | |
| | | | | | | | | | IAFAQYLQ | | |
| 371 | serum | 1127 | VFRR- | 1559 | ENFK- | 2046 | SAYSRGVFRR | 2469 | DAHKSEVAHRFK | 2964 | ALVLIAFAQY |
| | albumin | | DAHK | | ALVL | | | | DLGEENFK | | |
| 372 | serum | 1128 | VFRR- | 1560 | ALVL- | 2047 | SAYSRGVFRR | 2470 | DAHKSEVAHRFK | 2965 | IAFAQYLQQC |
| | albumin | | DAHK | | IAFA | | | | DLGEENFKALVL | | |
| 373 | trans- | 1129 | GLQM- | | N/A | 2048 | EGKNVIGLQM | | N/A | 2966 | GTNRGASQAG |
| | gelin-2 | | GTNR | | (end | | | | | | MTGYGMPRQI |
| | | | | | of | | | | | | L |
| | | | | | pro- | | | | | | |
| | | | | | tein) | | | | | | |
| 374 | trans- | 1130 | IGLQ- | | N/A | 2049 | QEGKNVIGLQ | | N/A | 2967 | MGTNRGASQA |
| | gelin-2 | | MGTN | | (end | | | | | | GMTGYGMPRQ |
| | | | | | of | | | | | | IL |
| | | | | | pro- | | | | | | |
| | | | | | tein) | | | | | | |
| 375 | trans- | 1131 | QAGM- | | N/A | 2050 | TNRGASQAGM | | N/A | 2968 | TGYGMPRQIL |
| | gelin-2 | | TGYG | | (end | | | | | | |
| | | | | | of | | | | | | |
| | | | | | pro- | | | | | | |
| | | | | | tein) | | | | | | |
| 376 | pancreatic | 1132 | YGKR- | 1561 | AVPR- | 2051 | MLTRPRYGKR | 2471 | HKEDTLAFSEWG | 2969 | ELSPLDL |
| | prohormone | | HKED | | ELSP | | | | SPHAAVPR | | |
| 377 | pancreatic | 1133 | QGAP- | 1562 | LRRY- | 2052 | QPLLGAQGAP | 2472 | LEPVYPGDNATP | 2970 | INMLTRPRYG |
| | prohormone | | LEPV | | INML | | | | EQMAQYAADLRR | | |
| | | | | | | | | | Y | | |
| 378 | pancreatic | 1134 | PLLG- | 1563 | RPRY- | 2053 | VALLLQPLLG | 2473 | AQGAPLEPVYPG | 2971 | GKRHKEDTLA |
| | prohormone | | AQGA | | GKRH | | | | DNATPEQMAQYA | | |
| | | | | | | | | | ADLRRYINMLTR | | |
| | | | | | | | | | PRY | | |
| 379 | pancreatic | 1135 | QGAP- | 1564 | RPRY- | 2054 | QPLLGAQGAP | 2474 | LEPVYPGDNATP | 2972 | GKRHKEDTLA |
| | prohormone | | LEPV | | GKRH | | | | EQMAQYAADLRR | | |
| | | | | | | | | | YINMLTRPRY | | |
| 380 | neurosec- | 1136 | AAPP- | 1565 | VRGA- | 2055 | LINGLGAAPP | 2475 | GRPEAQPPPLSS | 2973 | RNSEPQDEGE |
| | retory | | GRPE | | RNSE | | | | EHKEPVAGDAVP | | |
| | protein | | | | | | | | GPKDGSAPEVRG | | |
| | VGF | | | | | | | | A | | |
| 381 | neurosec- | 1137 | AAPP- | 1566 | APEV- | 2056 | LINGLGAAPP | 2476 | GRPEAQPPPLSS | 2974 | RGARNSEPQD |
| | retory | | GRPE | | RGAR | | | | EHKEPVAGDAVP | | |
| | protein | | | | | | | | GPKDGSAPEV | | |
| | VGF | | | | | | | | | | |
| 382 | neurosec- | 1138 | GLGA- | 1567 | VRGA- | 2057 | CLLLINGLGA | 2477 | APPGRPEAQPPP | 2975 | RNSEPQDEGE |
| | retory | | APPG | | RNSE | | | | LSSEHKEPVAGD | | |
| | protein | | | | | | | | AVPGPKDGSAPE | | |
| | VGF | | | | | | | | VRGA | | |
| 383 | neurosec- | 1139 | RKKN- | 1568 | PTHV- | 2058 | VEEKRKRKKN | 2478 | APPEPVPPPRAA | 2976 | RSPQPPPPAP |
| | retory | | APPE | | RSPQ | | | | PAPTHV | | |
| | protein | | | | | | | | | | |
| | VGF | | | | | | | | | | |
| 384 | neurosec- | 1140 | KRKK- | 1569 | PTHV- | 2059 | EVEEKRKRKK | 2479 | NAPPEPVPPPRA | 2977 | RSPQPPPPAP |
| | retory | | NAPP | | RSPQ | | | | APAPTHV | | |
| | protein | | | | | | | | | | |
| | VGF | | | | | | | | | | |
| 385 | neurosec- | 1141 | GLGA- | 1570 | APEV- | 2060 | CLLLINGLGA | 2480 | APPGRPEAQPPP | 2978 | RGARNSEPQD |
| | retory | | APPG | | RGAR | | | | LSSEHKEPVAGD | | |
| | protein | | | | | | | | AVPGPKDGSAPE | | |
| | VGF | | | | | | | | V | | |
| 386 | neurosec- | 1142 | EEEA- | 1571 | LTET- | 2061 | GSQQGPEEEA | 2481 | AEALLTET | 2979 | VRSQTHSLPA |
| | retory | | AEAL | | VRSQ | | | | | | |
| | protein | | | | | | | | | | |
| | VGF | | | | | | | | | | |
| 387 | neurosec- | 1143 | KRKK- | 1572 | ELPD- | 2062 | EVEEKRKRKK | 2482 | NAPPEPVPPPRA | 2980 | WNEVLPPWDR |
| | retory | | NAPP | | WNEV | | | | APAPTHVRSPQP | | |
| | protein | | | | | | | | PPPAPAPARDEL | | |
| | VGF | | | | | | | | PD | | |
| 388 | cerulo- | 1144 | LLHC- | 1573 | EDTK- | 2063 | RTPGIWLLHC | 2483 | HVTDHIHAGMET | | N/A |
| | plasmin | | HVTD | | SG | | | | TYTVLQNEDTK | | |
| 389 | cerulo- | 1145 | PAWA- | 1574 | WDYA- | 2064 | LFLCSTPAWA | 2484 | KEKHYYIGIIET | 2981 | SDHGEKKLIS |
| | plasmin | | KEKH | | SDHG | | | | TWDYA | | |
| 390 | PDZ and | 1146 | PFTA- | 1575 | TNQY- | 2065 | HNRSAMPFTA | 2485 | SPASSTTARVIT | 2982 | NNPAGLYSSE |
| | LIM | | SPAS | | NNPA | | | | NQY | | |
| | domain | | | | | | | | | | |
| | protein 1 | | | | | | | | | | |
| 391 | PDZ and | 1147 | LVLQ- | 1576 | APVT- | 2066 | KQSTSFLVLQ | 2486 | EILESEEKGDPN | 2983 | KVAASIGNAQ |
| | LIM | | EILE | | KVAA | | | | KPSGFRSVKAPV | | |
| | domain | | | | | | | | T | | |
| | protein 1 | | | | | | | | | | |
| 392 | PDZ and | 1148 | TNQY- | 1577 | SGVE- | 2067 | TTARVITNQY | 2487 | NNPAGLYSSENI | 2984 | ANSRPLDHAQ |
| | LIM | | NNPA | | ANSR | | | | SNFNNALESKTA | | |
| | domain | | | | | | | | ASGVE | | |
| | protein 1 | | | | | | | | | | |
| 393 | tubulin | 1149 | FPLA- | 1578 | YHEQ- | 2068 | PYPRIHFPLA | 2488 | TYAPVISAEKAY | 2985 | LSVAEITNAC |
| | alpha-4A | | TYAP | | LSVA | | | | HEQ | | |
| | chain | | | | | | | | | | |
| 394 | tubulin | 1150 | FPLA- | 1579 | HEQL- | 2069 | PYPRIHFPLA | 2489 | TYAPVISAEKAY | 2986 | SVAEITNACE |
| | alpha-4A | | TYAP | | SVAE | | | | HEQL | | |
| | chain | | | | | | | | | | |
| 395 | tubulin | 1151 | PVIS- | 1580 | EITN- | 2070 | PLATYAPVIS | 2490 | AEKAYHEQLSVA | 2987 | ACFEPANQMV |
| | alpha-4A | | AEKA | | ACFE | | | | EITN | | |
| | chain | | | | | | | | | | |
| 396 | tubulin | 1152 | M- | 1581 | LGRL- | | N/A | 2491 | VHLTPEEKSAVT | 2988 | LVVYPWTQRF |
| | alpha-4A | | VHLT | | LVVY | | | | ALWGKVNVDEVG | | |
| | chain | | | | | | | | GEALGRL | | |
| 397 | tubulin | 1153 | M- | 1582 | TQRF- | | N/A | 2492 | VHLTPEEKSAVT | 2989 | FESFGDLSTP |
| | alpha-4A | | VHLT | | FESF | | | | ALWGKVNVDEVG | | |
| | chain | | | | | | | | GEALGRLLVVYP | | |
| | | | | | | | | | WTQRF | | |
| 398 | tubulin | 1154 | LGRL- | 1583 | TQRF- | 2071 | EVGGEALGRL | 2493 | LVVYPWTQRF | 2990 | FESFGDLSTP |
| | alpha-4A | | LVVY | | FESF | | | | | | |
| | chain | | | | | | | | | | |
| 399 | multi- | 1155 | SLNT- | 1584 | RAPR- | 2072 | SNEQATSLNT | 2494 | VGGTGGIGGVGG | 2991 | ETYLSRGDSS |
| | merin-1 | | VGGT | | ETYL | | | | TGGVGNRAPR | | |
| 400 | multi- | 1156 | LNTV- | 1585 | RAPR- | 2073 | NEQATSLNTV | 2495 | GGTGGIGGVGGT | 2992 | ETYLSRGDSS |
| | merin-1 | | GGTG | | ETYL | | | | GGVGNRAPR | | |
| 401 | multi- | 1157 | SLNT- | 1586 | NRAP- | 2074 | SNEQATSLNT | 2496 | VGGTGGIGGVGG | 2993 | RETYLSRGDS |
| | merin-1 | | VGGT | | RETY | | | | TGGVGNRAP | | |
| 402 | multi- | 1158 | TSLN- | 1587 | RAPR- | 2075 | KSNEQATSLN | 2497 | TVGGTGGIGGVG | 2994 | ETYLSRGDSS |
| | merin-1 | | TVGG | | ETYL | | | | GTGGVGNRAPR | | |
| 403 | inter- | 1159 | EVSG- | 1588 | RRYQ- | 2076 | ICFFLSEVSG | 2498 | FEIPINGLSEFV | 2995 | RSLPGESEEM |
| | alpha- | | FEIP | | RSLP | | | | DYEDLVELAPGK | | |
| | trypsin | | | | | | | | FQLVAENRRYQ | | |
| | inhibitor | | | | | | | | | | |
| | heavy | | | | | | | | | | |
| | chain H2 | | | | | | | | | | |
| 404 | inter- | 1160 | RITR- | 1589 | RMLA- | 2077 | TAAAKRRITR | 2499 | SILQMSLDHHIV | 2996 | DAPPQDPSCC |
| | alpha- | | SILQ | | DAPP | | | | TPLTSLVIENEA | | |
| | trypsin | | | | | | | | GDERMLA | | |
| | inhibitor | | | | | | | | | | |
| | heavy | | | | | | | | | | |
| | chain H2 | | | | | | | | | | |
| 405 | clusterin | 1161 | VTTV- | 1590 | PVEV- | 2078 | DQYYLRVTTV | 2500 | ASHTSDSDVPSG | 2997 | SRKNPKFMET |
| | | | ASHT | | SRKN | | | | VTEVVVKLFDSD | | |
| | | | | | | | | | PITVTVPVEV | | |
| 406 | clusterin | 1162 | KNPK- | | N/A | 2079 | PVEVSRKNPK | | N/A | 2998 | FMETVAEKAL |
| | | | FMET | | (end | | | | | | QEYRKKHREE |
| | | | | | of | | | | | | |
| | | | | | pro- | | | | | | |
| | | | | | tein) | | | | | | |
| 407 | apolipo- | 1163 | PAQG- | | N/A | 2080 | VLEGPAPAQG | | N/A | 2999 | TPDVSSALDK |
| | protein | | TPDV | | (end | | | | | | LKEFGNTLED |
| | C-I | | | | of | | | | | | KARELISRIK |
| | | | | | pro- | | | | | | QSELSAKMRE |
| | | | | | tein) | | | | | | WFSETFQKVK |
| | | | | | | | | | | | EKLKIDS |
| 408 | apolipo- | 1164 | QGTP- | | N/A | 2081 | EGPAPAQGTP | | N/A | 3000 | DVSSALDKLK |
| | protein | | DVSS | | (end | | | | | | EFGNTLEDKA |
| | C-I | | | | of | | | | | | RELISRIKQS |
| | | | | | pro- | | | | | | ELSAKMREWF |
| | | | | | tein) | | | | | | SETFQKVKEK |
| | | | | | | | | | | | LKIDS |
| 409 | fibrinogen | 1165 | QLIK- | 1591 | ATLK- | 2082 | KTSEVKQLIK | 2501 | AIQLTYNPDESS | 3001 | SRKMLEEIMK |
| | gamma | | AIQL | | SRKM | | | | KPNMIDAATLK | | |
| | chain | | | | | | | | | | |
| 410 | fibrinogen | 1166 | EGFG- | 1592 | HLIS- | 2083 | NWIQYKEGFG | 2502 | HLSPTGTTEFWL | 3002 | TQSAIPYALR |
| | gamma | | HLSP | | TQSA | | | | GNEKIHLIS | | |
| | chain | | | | | | | | | | |
| 411 | fibrinogen | 1167 | NRLT- | | N/A | 2084 | MKIIPFNRLT | | N/A | 3003 | IGEGQQHHLG |
| | gamma | | IGEG | | (end | | | | | | GAKQAGDV |
| | chain | | | | of | | | | | | |
| | | | | | pro- | | | | | | |
| | | | | | tein) | | | | | | |
| 412 | N-acetyl- | 1168 | RSRR- | | N/A | 2085 | ARSVSKRSRR | | N/A | 3004 | EPPPRTLPAT |
| | muramoyl- | | EPPP | | (end | | | | | | DLQ |
| | L-alanine | | | | of | | | | | | |
| | amidase | | | | pro- | | | | | | |
| | | | | | tein) | | | | | | |
| 413 | N-acetyl- | 1169 | SRRE- | | N/A | 2086 | RSVSKRSRRE | | N/A | 3005 | PPPRTLPATD |
| | muramoyl- | | PPPR | | (end | | | | | | LQ |
| | L-alanine | | | | of | | | | | | |
| | amidase | | | | pro- | | | | | | |
| | | | | | tein) | | | | | | |
| 414 | immuno- | 1170 | GSVT- | 1593 | PSVS- | 2087 | LLSHCTGSVT | 2503 | SYVLTQPPSVS | 3006 | VAPGQTARIT |
| | globulin | | SYVL | | VAPG | | | | | | |
| | lambda | | | | | | | | | | |
| | variable | | | | | | | | | | |
| | 3-21 | | | | | | | | | | |
| 415 | immuno- | 1171 | SVTS- | 1594 | PSVS- | 2088 | LSHCTGSVTS | 2504 | YVLTQPPSVS | 3007 | VAPGQTARIT |
| | globulin | | YVLT | | VAPG | | | | | | |
| | lambda | | | | | | | | | | |
| | variable | | | | | | | | | | |
| | 3-21 | | | | | | | | | | |
| 416 | histone | 1172 | M- | 1595 | KKKA- | | N/A | 2505 | SETAPAAPAAPA | 3008 | RKSAGAAKRK |
| | H1.4 | | SETA | | RKSA | | | | PAEKTPVKKKA | | |
| 417 | histone | 1173 | M- | 1596 | KTPV- | | N/A | 2506 | SETAPAAPAAPA | 3009 | KKKARKSAGA |
| | H1.4 | | SETA | | KKKA | | | | PAEKTPV | | |
| 418 | histone | 1174 | M- | 1597 | TPVK- | | N/A | 2507 | SETAPAAPAAPA | 3010 | KKARKSAGAA |
| | H1.4 | | SETA | | KKAR | | | | PAEKTPVK | | |
| 419 | adhesion | 1175 | CNHF- | 1598 | RSAS- | 2089 | SETVCLCNHF | 2508 | THFGVLMDLPRS | 3011 | QLDARNTKVL |
| | G-protein | | THFG | | QLDA | | | | AS | | |
| | coupled | | | | | | | | | | |
| | receptor | | | | | | | | | | |
| | G6 | | | | | | | | | | |
| 420 | adhesion | 1176 | CNHF- | 1599 | QLDA- | 2090 | SETVCLCNHF | 2509 | THFGVLMDLPRS | 3012 | RNTKVLTFIS |
| | G-protein | | THFG | | RNTK | | | | ASQLDA | | |
| | coupled | | | | | | | | | | |
| | receptor | | | | | | | | | | |
| | G6 | | | | | | | | | | |
| 421 | immuno- | 1177 | SEAS- | 1600 | PSVS- | 2091 | LTLCTGSEAS | 2510 | YELTQPPSVS | 3013 | VSPGQTARIT |
| | globulin | | YELT | | VSPG | | | | | | |
| | lambda | | | | | | | | | | |
| | variable | | | | | | | | | | |
| | 3-25 | | | | | | | | | | |
| 422 | immuno- | 1178 | GSVA- | 1601 | PSVS- | 2092 | VLAYCTGSVA | 2511 | SYELTQPPSVS | 3014 | VSPGQTASIT |
| | globulin | | SYEL | | VSPG | | | | | | |
| | lambda | | | | | | | | | | |
| | variable | | | | | | | | | | |
| | 3-25 | | | | | | | | | | |
| 423 | immuno- | 1179 | SWAQ- | 1602 | PSVS- | 2093 | LIHCTGSWAQ | 2512 | SVLTQPPSVS | 3015 | AAPGQKVTIS |
| | globulin | | SVLT | | AAPG | | | | | | |
| | lambda | | | | | | | | | | |
| | variable | | | | | | | | | | |
| | 1-51 | | | | | | | | | | |
| 424 | immuno- | 1180 | QSVL- | 1603 | PSVS- | 2094 | CTGSWAQSVL | 2513 | TQPPSVS | 3016 | AAPGQKVTIS |
| | globulin | | TQPP | | AAPG | | | | | | |
| | lambda | | | | | | | | | | |
| | variable | | | | | | | | | | |
| | 1-51 | | | | | | | | | | |
| 425 | immuno- | 1181 | GSWA- | 1604 | PSVS- | 2095 | LITHCAGSWA | 2514 | QSVLTQPPSVS | 3017 | EAPRQRVTIS |
| | globulin | | QSVL | | EAPR | | | | | | |
| | lambda | | | | | | | | | | |
| | variable | | | | | | | | | | |
| | 1-36 | | | | | | | | | | |
| 426 | immuno- | 1182 | SWAQ- | 1605 | PSVS- | 2096 | LITHCAGSWA | 2515 | SVLTQPPSVS | 3018 | EAPRQRVTIS |
| | globulin | | SVLT | | EAPR | | Q | | | | |
| | lambda | | | | | | | | | | |
| | variable | | | | | | | | | | |
| | 1-36 | | | | | | | | | | |
| 427 | immuno- | 1183 | SYEL- | 1606 | PSVS- | 2097 | CTGSVASYEL | 2516 | TQPPSVS | 3019 | VSPGQTASIT |
| | globulin | | TQPP | | VSPG | | | | | | |
| | lambda | | | | | | | | | | |
| | variable | | | | | | | | | | |
| | 1-36 | | | | | | | | | | |
| 428 | mannan- | 1184 | GSVA- | 1607 | GRLA- | 2098 | LLGLLCGSVA | 2517 | TPLGPKWPEPVF | 3020 | SPGFPGEYAN |
| | binding | | TPLG | | SPGF | | | | GRLA | | |
| | lectin | | | | | | | | | | |
| | serine | | | | | | | | | | |
| | protease 2 | | | | | | | | | | |
| 429 | immuno- | 1185 | DTTG- | 1608 | GTLS- | 2099 | LLLWLPDTTG | 2518 | EIVLTQSPGTLS | 3021 | LSPGERATLS |
| | globulin | | EIVL | | LSPG | | | | | | |
| | kappa | | | | | | | | | | |
| | variable | | | | | | | | | | |
| | 3-20 | | | | | | | | | | |
| 430 | immuno- | 1186 | TTGE- | 1609 | GTLS- | 2100 | LLWLPDTTGE | 2519 | IVLTQSPGTLS | 3022 | LSPGERATLS |
| | globulin | | IVLT | | LSPG | | | | | | |
| | kappa | | | | | | | | | | |
| | variable | | | | | | | | | | |
| | 3-20 | | | | | | | | | | |
| 431 | immuno- | 1187 | GSSG- | 1610 | LPVT- | 2101 | LMLWVPGSSG | 2520 | DVVMTQSPLSLP | 3023 | LGQPASISCR |
| | globulin | | DVVM | | LGQP | | | | VT | | |
| | kappa | | | | | | | | | | |
| | variable | | | | | | | | | | |
| | 2-30 | | | | | | | | | | |
| 432 | immuno- | 1188 | GSSG- | 1611 | SPLS- | 2102 | LMLWVPGSSG | 2521 | DVVMTQSPLS | 3024 | LPVTLGQPAS |
| | globulin | | DVVM | | LPVT | | | | | | |
| | kappa | | | | | | | | | | |
| | variable | | | | | | | | | | |
| | 2-30 | | | | | | | | | | |
| 433 | insulin- | 1189 | PVGK- | 1612 | QSTQ- | 2103 | DNFPRYPVGK | 2522 | FFQYDTWKQSTQ | 3025 | RLRRGLPALL |
| | like | | FFQY | | RLRR | | | | | | |
| | growth | | | | | | | | | | |
| | factor II | | | | | | | | | | |
| 434 | insulin- | 1190 | PVGK- | 1613 | TQRL- | 2104 | DNFPRYPVGK | 2523 | FFQYDTWKQSTQ | 3026 | RRGLPALLRA |
| | like | | FFQY | | RRGL | | | | RL | | |
| | growth | | | | | | | | | | |
| | factor II | | | | | | | | | | |
| 435 | apolipo- | 1191 | VNFL- | 1614 | TQPA- | 2105 | KAGTELVNFL | 2524 | SYFVELGTQPA | | N/A |
| | protein | | SYFV | | TQ | | | | | | |
| | A-II | | | | | | | | | | |
| 436 | apolipo- | 1192 | IKKA- | 1615 | TQPA- | 2106 | EQLTPLIKKA | 2525 | GTELVNFLSYFV | | N/A |
| | protein | | GTEL | | TQ | | | | ELGTQPA | | |
| | A-II | | | | | | | | | | |
| 437 | apolipo- | 1193 | FQTV- | 1616 | SPEL- | 2107 | SLVSQYFQTV | 2526 | TDYGKDLMEKVK | 3027 | QAEAKSYFEK |
| | protein | | TDYG | | QAEA | | | | SPEL | | |
| | A-II | | | | | | | | | | |
| 438 | probable | 1194 | GSSG- | 1617 | SPVT- | 2108 | LMLWVPGSSG | 2527 | DIVMTQTPLSSP | 3028 | LGQPASISFR |
| | non- | | DIVM | | LGQP | | | | VT | | |
| | functional | | | | | | | | | | |
| | immuno- | | | | | | | | | | |
| | globulin | | | | | | | | | | |
| | kappa | | | | | | | | | | |
| | variable | | | | | | | | | | |
| | 2D-24 | | | | | | | | | | |
| 439 | probable | 1195 | GSSG- | 1618 | TPLS- | 2109 | LMLWVPGSSG | 2528 | DIVMTQTPLS | 3029 | SPVTLGQPAS |
| | non- | | DIVM | | SPVT | | | | | | |
| | functional | | | | | | | | | | |
| | immuno- | | | | | | | | | | |
| | globulin | | | | | | | | | | |
| | kappa | | | | | | | | | | |
| | variable | | | | | | | | | | |
| | 2D-24 | | | | | | | | | | |
| 440 | pro- | 1196 | RTAT- | 1619 | FNPR- | 2110 | DRAIEGRTAT | 2529 | SEYQTFFNPR | 3030 | TFGSGEADCG |
| | thrombin | | SEYQ | | TFGS | | | | | | |
| 441 | pro- | 1197 | TATS- | 1620 | FNPR- | 2111 | RAIEGRTATS | 2530 | EYQTFFNPR | 3031 | TFGSGEADCG |
| | thrombin | | EYQT | | TFGS | | | | | | |
| 442 | pro- | 1198 | LVHS- | 1621 | LQRV- | 2112 | LAALCSLVHS | 2531 | QHVFLAPQQARS | 3032 | RRANTFLEEV |
| | thrombin | | QHVF | | RRAN | | | | LLQRV | | |
| 443 | coagu- | 1199 | SAEC- | 1622 | NRPK- | 2113 | LLGYLLSAEC | 2532 | TVFLDHENANKI | 3033 | RYNSGKLEEF |
| | lation | | TVFL | | RYNS | | | | LNRPK | | |
| | factor IX | | | | | | | | | | |
| 444 | coagu- | 1200 | ECTV- | 1623 | NRPK- | 2114 | GYLLSAECTV | 2533 | FLDHENANKILN | 3034 | RYNSGKLEEF |
| | lation | | FLDH | | RYNS | | | | RPK | | |
| | factor IX | | | | | | | | | | |
| 445 | apolipo- | 1201 | GVRA- | 1624 | KPLG- | 2115 | ALFLGVGVRA | 2534 | EEAGARVQQNVP | 3035 | DWAAGTMDPE |
| | protein L1 | | EEAG | | DWAA | | | | SGTDTGDPQSKP | | |
| | | | | | | | | | LG | | |
| 446 | apolipo- | 1202 | GVRA- | 1625 | QSKP- | 2116 | ALFLGVGVRA | 2535 | EEAGARVQQNVP | 3036 | LGDWAAGTMD |
| | protein L1 | | EEAG | | LGDW | | | | SGTDTGDPQSKP | | |
| 447 | deleted in | 1203 | RSKR- | | N/A | 2117 | YRGCVLRSKR | | N/A | 3037 | DVGSYQEKVD |
| | malignant | | DVGS | | (end | | | | | | VVLGPIQLQT |
| | brain | | | | of | | | | | | PPRREEEPR |
| | tumors 1 | | | | pro- | | | | | | |
| | protein | | | | tein) | | | | | | |
| 448 | desmo- | 1204 | GELR- | 1626 | KRRQ- | 2118 | VVILVHGELR | 2536 | IETKGQYDEEEM | 3038 | KREWVKFAKP |
| | glein-3 | | IETK | | KREW | | | | TMQQAKRRQ | | |
| 449 | desmo- | 1205 | LVHG- | 1627 | KRRQ- | 2119 | IFVVVILVHG | 2537 | ELRIETKGQYDE | 3039 | KREWVKFAKP |
| | glein-3 | | ELRI | | KREW | | | | EEMTMQQAKRRQ | | |
| 450 | calsyn- | 1206 | NHMA- | 1628 | PHPF- | 2120 | NPMEHANHMA | 2538 | AQPQFVHPEHRS | 3040 | AVVPSTATVV |
| | tenin-1 | | AQPQ | | AVVP | | | | FVDLSGHNLANP | | |
| | | | | | | | | | HPF | | |
| 451 | calsyn- | 1207 | HANH- | 1629 | PHPF- | 2121 | TANPMEHANH | 2539 | MAAQPQFVHPEH | 3041 | AVVPSTATVV |
| | tenin-1 | | MAAQ | | AVVP | | | | RSFVDLSGHNLA | | |
| | | | | | | | | | NPHPF | | |
| 452 | immuno- | 1208 | GAVT- | 1630 | AGVE- | 2122 | ISDFYPGAVT | 2540 | VAWKADSSPVKA | 3042 | TTTPSKQSNN |
| | globulin | | VAWK | | TTTP | | | | GVE | | |
| | lambda | | | | | | | | | | |
| | constant 3 | | | | | | | | | | |
| 453 | immuno- | 1209 | GAVT- | 1631 | SYLS- | 2123 | ISDFYPGAVT | 2541 | VAWKADSSPVKA | 3043 | LTPEQWKSHK |
| | globulin | | VAWK | | LTPE | | | | GVETTTPSKQSN | | |
| | lambda | | | | | | | | NKYAASSYLS | | |
| | constant 3 | | | | | | | | | | |
| 454 | immuno- | 1210 | SPVK- | 1632 | SYLS- | 2124 | AWKADSSPVK | 2542 | AGVETTTPSKQS | 3044 | LTPEQWKSHK |
| | globulin | | AGVE | | LTPE | | | | NNKYAASSYLS | | |
| | lambda | | | | | | | | | | |
| | constant 3 | | | | | | | | | | |
| 455 | complement | 1211 | KTWG- | 1633 | FRVG- | 2125 | FLIFLGKTWG | 2543 | QEQTYVISAPKI | 3045 | ASENIVIQVY |
| | C5 | | QEQT | | ASEN | | | | FRVG | | |
| 456 | alpha-2- | 1212 | NKVD- | 1634 | LRVT- | 2126 | VENCLANKVD | 2544 | LSFSPSQSLPAS | 3046 | AAPQSVCALR |
| | macro- | | LSFS | | AAPQ | | | | HAHLRVT | | |
| | globulin | | | | | | | | | | |
| 457 | alpha-2- | 1213 | PTDA- | 1635 | SLLH- | 2127 | LLLVLLPTDA | 2545 | SVSGKPQYMVLV | 3047 | TETTEKGCVL |
| | macro- | | SVSG | | TETT | | | | PSLLH | | |
| | globulin | | | | | | | | | | |
| 458 | myosin-9 | 1214 | M- | 1636 | DKNF- | | N/A | 2546 | AQQAADKYLYVD | 3048 | INNPLAQADW |
| | | | AQQA | | INNP | | | | KNF | | |
| 459 | sodium/ | 1215 | M- | 1637 | NGGL- | | N/A | 2547 | TGLSMDGGGSPK | 3049 | IFAGLAFIVG |
| | potassium- | | TGLS | | IFAG | | | | GDVDPFYYDYET | | |
| | transport- | | | | | | | | VRNGGL | | |
| | ing | | | | | | | | | | |
| | ATPase | | | | | | | | | | |
| | subunit | | | | | | | | | | |
| | gamma | | | | | | | | | | |
| 460 | sodium/ | 1216 | M- | 1638 | DVDP- | | N/A | 2548 | TGLSMDGGGSPK | 3050 | FYYDYETVRN |
| | potassium- | | TGLS | | FYYD | | | | GDVDP | | |
| | transport- | | | | | | | | | | |
| | ing | | | | | | | | | | |
| | ATPase | | | | | | | | | | |
| | subunit | | | | | | | | | | |
| | gamma | | | | | | | | | | |
| 461 | immuno- | 1217 | GSSG- | 1639 | SPLS- | 2128 | LMLWVSGSSG | 2549 | DIVMTQSPLS | 3051 | LPVTPGEPAS |
| | globulin | | DIVM | | LPVT | | | | | | |
| | kappa | | | | | | | | | | |
| | variable | | | | | | | | | | |
| | 2-28 | | | | | | | | | | |
| 462 | immuno- | 1218 | GSSG- | 1640 | LPVT- | 2129 | LMLWVSGSSG | 2550 | DIVMTQSPLSLP | 3052 | PGEPASISCR |
| | globulin | | DIVM | | PGEP | | | | VT | | |
| | kappa | | | | | | | | | | |
| | variable | | | | | | | | | | |
| | 2-28 | | | | | | | | | | |
| 463 | onco- | 1219 | RMRR- | | N/A | 2130 | AQGCHRRMRR | | N/A | 3053 | GAGGEDSAGL |
| | protein- | | GAGG | | (end | | | | | | QGQTLTGGPI |
| | induced | | | | of | | | | | | RIDWED |
| | transcript | | | | pro- | | | | | | |
| | 3 protein | | | | tein) | | | | | | |
| 464 | serglycin | 1220 | SDAF- | | N/A | 2131 | YQLVDESDAF | | N/A | 3054 | HDNLRSLDRN |
| | | | HDNL | | (end | | | | | | LPSDSQDLGQ |
| | | | | | of | | | | | | HGLEEDFML |
| | | | | | pro- | | | | | | |
| | | | | | tein) | | | | | | |
| 465 | coagula- | 1221 | GDRN- | | N/A | 2132 | SWGSGCGDRN | | N/A | 3055 | KPGVYTDVAY |
| | tion | | KPGV | | (end | | | | | | YLAWIREHTV |
| | factor XII | | | | of | | | | | | S |
| | | | | | pro- | | | | | | |
| | | | | | tein) | | | | | | |
| 466 | coagula- | 1222 | M- | 1641 | VVPR- | | N/A | 2551 | SETSRTAFGGRR | 3056 | GVNLQEFLNV |
| | tion | | SETS | | GVNL | | | | AVPPNNSNAAED | | |
| | factor | | | | | | | | DLPTVELQGVVP | | |
| | XIII | | | | | | | | R | | |
| | A chain | | | | | | | | | | |
| 467 | insulin | 1223 | KTRR- | 1642 | GSLQ- | 2133 | GFFYTPKTRR | 2552 | EAEDLQVGQVEL | 3057 | KRGIVEQCCT |
| | | | EAED | | KRGI | | | | GGGPGAGSLQPL | | |
| | | | | | | | | | ALEGSLQ Q | | |
| 468 | histidine- | 1224 | GKFK- | | N/A | 2134 | VSESCPGKFK | | N/A | 3058 | SGFPQVSMFF |
| | rich | | SGFP | | (end | | | | | | THTFPK |
| | glyco- | | | | of | | | | | | |
| | protein | | | | pro- | | | | | | |
| | | | | | tein) | | | | | | |
| 469 | immuno- | 1225 | DTTG- | 1643 | ATLS- | 2135 | LLLWLPDTTG | 2553 | EIVLTQSPATLS | 3059 | LSPGERATLS |
| | globulin | | EIVL | | LSPG | | | | | | |
| | kappa | | | | | | | | | | |
| | variable | | | | | | | | | | |
| | 3-11 | | | | | | | | | | |
| 470 | immuno- | 1226 | GARC- | 1644 | SSLS- | 2136 | LLLWLRGARC | 2554 | DIQMTQSPSSLS | 3060 | ASVGDRVTIT |
| | globulin | | DIQM | | ASVG | | | | | | |
| | kappa | | | | | | | | | | |
| | variable | | | | | | | | | | |
| | 1-3 | | | | | | | | | | |
| 471 | collagen | 1227 | AGFD- | 1645 | QPPQ- | 2137 | PPGPPSAGFD | 2555 | FSFLPQPPQ | 3061 | EKAHDGGRYY |
| | alpha-1(1) | | FSFL | | EKAH | | | | | | |
| | chain | | | | | | | | | | |
| 472 | inter- | 1228 | CVGS- | 1646 | RVPR- | 2138 | CLGLSLCVGS | 2556 | QEEAQSWGHSSE | 3062 | QVRLLQRLKT |
| | alpha- | | QEEA | | QVRL | | | | QDGLRVPR | | |
| | trypsin | | | | | | | | | | |
| | inhibitor | | | | | | | | | | |
| | heavy | | | | | | | | | | |
| | chain H5 | | | | | | | | | | |
| 473 | latent- | 1229 | AGHA- | 1647 | AAKV- | 2139 | LALFVGAGHA | 2557 | QRDPVGRYEPAG | 3063 | YSLFREQDAP |
| | trans- | | QRDP | | YSLF | | | | GDANRLRRPGGS | | |
| | forming | | | | | | | | YPAAAAAKV | | |
| | growth | | | | | | | | | | |
| | factor | | | | | | | | | | |
| | beta- | | | | | | | | | | |
| | binding | | | | | | | | | | |
| | protein 2 | | | | | | | | | | |
| 474 | latent- | 1230 | AGHA- | 1648 | RPGG- | 2140 | LALFVGAGHA | 2558 | QRDPVGRYEPAG | 3064 | SYPAAAAAKV |
| | trans- | | QRDP | | SYPA | | | | GDANRLRRPGG | | |
| | forming | | | | | | | | | | |
| | growth | | | | | | | | | | |
| | factor | | | | | | | | | | |
| | beta- | | | | | | | | | | |
| | binding | | | | | | | | | | |
| | protein 2 | | | | | | | | | | |
| 475 | integrin | 1231 | RDRR- | 1649 | QPSR- | 2141 | HPAHHKRDRR | 2559 | QIFLPEPEQPSR | 3065 | LQDPVLVSCD |
| | alpha-IIb | | QIFL | | LQDP | | | | | | |
| 476 | membrane- | 1232 | LLYK- | 1650 | LPRL- | 2142 | LGLCIFLLYK | 2560 | IVRGDQPAASGD | 3066 | KRRDFTPAEL |
| | associated | | IVRG | | KRRD | | | | SDDDEPPPLPRL | | |
| | progester- | | | | | | | | | | |
| | one | | | | | | | | | | |
| | receptor | | | | | | | | | | |
| | component | | | | | | | | | | |
| | 1 | | | | | | | | | | |
| 477 | immuno- | 1233 | GSWA- | 1651 | HSVS- | 2143 | LLAHCTGSWA | 2561 | NFMLTQPHSVS | 3067 | ESPGKTVTIS |
| | globulin | | NFML | | ESPG | | | | | | |
| | lambda | | | | | | | | | | |
| | variable | | | | | | | | | | |
| | 6-57 | | | | | | | | | | |
| 478 | immuno- | 1234 | SWAN- | 1652 | HSVS- | 2144 | LAHCTGSWAN | 2562 | FMLTQPHSVS | 3068 | ESPGKTVTIS |
| | globulin | | FMLT | | ESPG | | | | | | |
| | lambda | | | | | | | | | | |
| | variable | | | | | | | | | | |
| | 6-57 | | | | | | | | | | |
| 479 | immuno- | 1235 | DTTG- | 1653 | ATLS- | 2145 | LLLWLPDTTG | 2563 | EIVMTQSPATLS | 3069 | VSPGERATLS |
| | globulin | | EIVM | | VSPG | | | | | | |
| | kappa | | | | | | | | | | |
| | variable | | | | | | | | | | |
| | 3-15 | | | | | | | | | | |
| 480 | complement | 1236 | PTRG- | 1654 | QQLT- | 2146 | GVLQACPTRG | 2564 | SVLLAQELPQQL | 3070 | SPGYPEPYGK |
| | C1r | | SVLL | | SPGY | | | | T | | |
| | sub- | | | | | | | | | | |
| | component- | | | | | | | | | | |
| | like | | | | | | | | | | |
| | protein | | | | | | | | | | |
| 481 | histone | 1237 | M- | 1655 | KKAA- | | N/A | 2565 | SETAPAAPAAAP | 3071 | KKAGGTPRKA |
| | H1.2 | | SETA | | KKAG | | | | PAEKAPVKKKAA | | |
| 482 | rho GDP- | 1238 | M- | 1656 | KLNY- | | N/A | 2566 | TEKAPEPHVEED | 3072 | KPPPQKSLKE |
| | dissoci- | | TEKA | | KPPP | | | | DDDELDSKLNY | | |
| | ation | | | | | | | | | | |
| | inhibitor | | | | | | | | | | |
| | 2 | | | | | | | | | | |
| 483 | latent- | 1239 | FARR- | 1657 | SRDT- | 2147 | PPPPGPFARR | 2567 | EAPYGAPRFDMP | 3073 | RRSFPEPEEP |
| | trans- | | EAPY | | RRSF | | | | DFEDDGGPYGES | | |
| | forming | | | | | | | | EAPAPPGPGTRW | | |
| | growth | | | | | | | | PYRSRDT | | |
| | factor | | | | | | | | | | |
| | beta- | | | | | | | | | | |
| | binding | | | | | | | | | | |
| | protein 4 | | | | | | | | | | |
| 484 | collagen | 1240 | PWRA- | 1658 | HHSS- | 2148 | PHPTARPWRA | 2568 | DDILASPPRLPE | 3074 | YVHLRPARPT |
| | alpha- | | DDIL | | YVHL | | | | PQPYPGAPHHSS | | |
| | 1(XVIII) | | | | | | | | | | |
| | chain | | | | | | | | | | |
| 485 | immuno- | 1241 | GSWA- | 1659 | PSVS- | 2149 | LLTQGTGSWA | 2569 | QSALTQPPSVS | 3075 | GSPGQSVTIS |
| | globulin | | QSAL | | GSPG | | | | | | |
| | lambda | | | | | | | | | | |
| | variable | | | | | | | | | | |
| | 2-18 | | | | | | | | | | |
| 486 | immuno- | 1242 | QSAL- | 1660 | PSVS- | 2150 | GTGSWAQSAL | 2570 | TQPPSVS | 3076 | GSPGQSVTIS |
| | globulin | | TQPP | | GSPG | | | | | | |
| | lambda | | | | | | | | | | |
| | variable | | | | | | | | | | |
| | 2-18 | | | | | | | | | | |
| 487 | zinc- | 1243 | SSLA- | | N/A | 2151 | SCHVQHSSLA | | N/A | 3077 | QPLVVPWEAS |
| | alpha-2- | | QPLV | | (end | | | | | | |
| | glycopro- | | | | of | | | | | | |
| | tein | | | | pro- | | | | | | |
| | | | | | tein) | | | | | | |
| 488 | talin-1 | 1244 | TVLQ- | 1661 | FQVG- | 2152 | VSPKKSTVLQ | 2571 | QQYNRVGKVEHG | 3078 | SMPPAQQQIT |
| | | | QQYN | | SMPP | | | | SVALPAIMRSGA | | |
| | | | | | | | | | SGPENFQVG | | |
| 489 | secreto- | 1245 | LRDP- | 1662 | HSRE- | 2153 | KFEVRLLRDP | 2572 | ADASEAHESSSR | 3079 | RADEPQWSLY |
| | granin-1 | | ADAS | | RADE | | | | GEAGAPGEEDIQ | | |
| | | | | | | | | | GPTKADTEKWAE | | |
| | | | | | | | | | GGGHSRE | | |
| 490 | neutrophil | 1246 | QAQA- | 1663 | PEQI- | 2154 | ILLVALQAQA | 2573 | EPLQARADEVAA | 3080 | AADIPEVVVS |
| | defensin 3 | | EPLQ | | AADI | | | | APEQI | | |
| 491 | cytochrome | 1247 | LYDN- | 1664 | PEKF- | 2155 | PTLDSVLYDN | 2574 | QEFPDPEKF | 3081 | KPEHFLNENG |
| | P450 2E1 | | QEFP | | KPEH | | | | | | |
| 492 | gastric | 1248 | VGLG- | 1665 | RGPR- | 2156 | LSLFLAVGLG | 2575 | EKKEGHFSALPS | 3082 | YAEGTFISDY |
| | inhibitory | | EKKE | | YAEG | | | | LPVGSHAKVSSP | | |
| | polypep- | | | | | | | | QPRGPR | | |
| | tide | | | | | | | | | | |
| 493 | immuno- | 1249 | GVQC- | 1666 | GSLR- | 2157 | LAAILKGVQC | 2576 | EVQLVESGGGLV | 3083 | LSCAASGFTF |
| | globulin | | EVQL | | LSCA | | | | KPGGSLR | | |
| | heavy | | | | | | | | | | |
| | variable | | | | | | | | | | |
| | 3-15 | | | | | | | | | | |
| 494 | immuno- | 1250 | SWAQ- | 1667 | RSVS- | 2158 | LTQGTGSWAQ | 2577 | SALTQPRSVS | 3084 | GSPGQSVTIS |
| | globulin | | SALT | | GSPG | | | | | | |
| | lambda | | | | | | | | | | |
| | variable | | | | | | | | | | |
| | 2-11 | | | | | | | | | | |
| 495 | trans- | 1251 | DIDC- | 1668 | PPPP- | 2159 | YDADCEDIDC | 2578 | KLMPPPPPP | 3085 | PGPMKKDKDQ |
| | cription | | KLMP | | PGPM | | | | | | |
| | initiation | | | | | | | | | | |
| | factor | | | | | | | | | | |
| | TFIID | | | | | | | | | | |
| | subunit 1 | | | | | | | | | | |
| 496 | collagen | 1252 | EQGR- | 1669 | PPGP- | 2160 | EKGERGEQGR | 2579 | DGPPGLPGTPGP | 3086 | KVSVDEPGPG |
| | alpha- | | DGPP | | KVSV | | | | PGPPGP | | |
| | 1(VII) | | | | | | | | | | |
| | chain | | | | | | | | | | |
| 497 | kinino- | 1253 | SLMK- | 1670 | FSPF- | 2161 | QPLGMISLMK | 2580 | RPPGFSPF | 3087 | RSSRIGEIKE |
| | gen-1 | | RPPG | | RSSR | | | | | | |
| 498 | integral | 1254 | AIRH- | 1671 | AVET- | 2162 | EASNCFAIRH | 2581 | FENKFAVET | 3088 | LICS |
| | membrane | | FENK | | LICS | | | | | | |
| | protein | | | | | | | | | | |
| | 2B | | | | | | | | | | |
| 499 | pigment | 1255 | QPAH- | | N/A | 2163 | TPSPGLQPAH | | N/A | 3089 | LTFPLDYHLN |
| | epithel- | | LTFP | | (end | | | | | | QPFIFVLRDT |
| | ium- | | | | of | | | | | | DTGALLFIGK |
| | derived | | | | pro- | | | | | | ILDPRGP |
| | factor | | | | tein) | | | | | | |
| 500 | voltage- | 1256 | RHRA- | 1672 | ADKE- | 2164 | GEEPARRHRA | 2582 | RHKAQPAHEAVE | 3090 | KELRNHQPRE |
| | dependent | | RHKA | | KELR | | | | KETTEKEATEKE | | |
| | N-type | | | | | | | | AEIVEADKE | | |
| | calcium | | | | | | | | | | |
| | channel | | | | | | | | | | |
| | subunit | | | | | | | | | | |
| | alpha-1B | | | | | | | | | | |
| 501 | immuno- | 1257 | SVAS- | 1673 | SSVS- | 2165 | LILCTVSVAS | 2583 | YELTQPSSVS | 3091 | VSPGQTARIT |
| | globulin | | YELT | | VSPG | | | | | | |
| | lambda | | | | | | | | | | |
| | variable | | | | | | | | | | |
| | 3-27 | | | | | | | | | | |
| 502 | ras | 1258 | PGGL- | 1674 | LSFQ- | 2166 | HPALNQPGGL | 2584 | QPLSFQ | 3092 | NPVYHLNNPI |
| | GTPase- | | QPLS | | NPVY | | | | | | |
| | activating | | | | | | | | | | |
| | protein | | | | | | | | | | |
| | nGAP | | | | | | | | | | |
| 503 | keratin, | 1259 | RQVR- | 1675 | HQTT- | 2167 | KEPVTTRQVR | 2585 | TIVEEVQDGKVI | | N/A |
| | type I | | TIVE | | R | | | | SSREQVHQTT | | |
| | cytoskel- | | | | | | | | | | |
| | etal 17 | | | | | | | | | | |
| 504 | tubulin | 1260 | MNTF- | 1676 | EPYN- | 2168 | EYPDRIMNTF | 2586 | SVVPSPKVSDTV | 3093 | ATLSVHQLVE |
| | beta chain | | SVVP | | ATLS | | | | VEPYN | | |
| 505 | sulfhydryl | 1261 | PGLR- | 1677 | WHLS- | 2169 | RPPKLHPGLR | 2587 | AAPGQEPPEHMA | 3094 | KRDTGAALLA |
| | oxidase 1 | | AAPG | | KRDT | | | | ELQRNEQEQPLG | | |
| | | | | | | | | | QWHLS | | |
| 506 | immuno- | 1262 | GAYG- | 1678 | SLAV- | 2170 | LLLWISGAYG | 2588 | DIVMTQSPDSLA | 3095 | SLGERATINC |
| | globulin | | DIVM | | SLGE | | | | V | | |
| | kappa | | | | | | | | | | |
| | variable | | | | | | | | | | |
| | 4-1 | | | | | | | | | | |
| 507 | complement | 1263 | RAGG- | 1679 | GEVT- | 2171 | VPALFCRAGG | 2589 | SIPIPQKLFGEV | 3096 | SPLFPKPYPN |
| | C1r sub- | | SIPI | | SPLF | | | | T | | |
| | component | | | | | | | | | | |
| 508 | homeobox | 1264 | KKPS- | 1680 | SPSP- | 2172 | KEKKSAKKPS | 2590 | QSATSPSP | 3097 | AASAVPASGV |
| | protein | | QSAT | | AASA | | | | | | |
| | Hox-B2 | | | | | | | | | | |
| 509 | trans- | 1265 | VALP- | 1681 | SPPG- | 2173 | ISKPPGVALP | 2591 | TVSPPG | 3098 | VDAKAQVKTE |
| | cription | | TVSP | | VDAK | | | | | | |
| | factor | | | | | | | | | | |
| | SOX-10 | | | | | | | | | | |
| 510 | E3 | 1266 | NKPC- | 1682 | TPSP- | 2174 | STGPSANKPC | 2592 | SKQPPPQPQHTP | 3099 | AAPPAAATIS |
| | ubiquitin- | | SKQP | | AAPP | | | | SP | | |
| | protein | | | | | | | | | | |
| | ligase | | | | | | | | | | |
| | SIAH2 | | | | | | | | | | |
| 511 | decorin | 1267 | GLDK- | | N/A | 2175 | VQCSDLGLDK | | N/A | 3100 | VPKDLPPDTT |
| | | | VPKD | | | | | | | | |
| 512 | SPARC | 1268 | HPVE- | | N/A | 2176 | RLEAGDHPVE | | N/A | 3101 | LLARDFEKNY |
| | | | LLAR | | | | | | | | |
| 513 | elastin | 1269 | LGYP- | | N/A | 2177 | PQPGVPLGYP | | N/A | 3102 | IKAPKLPGGY |
| | | | IKAP | | | | | | | | |
| 514 | elastin | 1270 | PGVV- | | N/A | 2178 | GGPGFGPGVV | | N/A | 3103 | GVPGAGVPGV |
| | | | GVPG | | | | | | | | |
| 515 | type I | 1271 | GVRG- | | N/A | 2179 | GSPGKDGVRG | | N/A | 3104 | LTGPIGPPGP |
| | collagen | | LTGP | | | | | | | | |
| | alpha-1 | | | | | | | | | | |
| | chain | | | | | | | | | | |
| 516 | type IV | 1272 | SDGL- | | N/A | 2180 | EPGPAGSDGL | | N/A | 3105 | PGLKGKRGDS |
| | collagen | | PGLK | | | | | | | | |
| | alpha-1 | | | | | | | | | | |
| | chain | | | | | | | | | | |
| 517 | laminin | 1273 | QAKN- | | N/A | 2181 | LNRKYEQAKN | | N/A | 3106 | ISQDLEKQAA |
| | gamma 1 | | ISQD | | | | | | | | |
| | chain | | | | | | | | | | |
| 518 | vimentin | 1274 | PGVR- | | N/A | 2182 | RLRSSVPGVR | | N/A | 3107 | LLQDSVDFSL |
| | | | LLQD | | | | | | | | |
| 519 | type III | 1275 | QGLQ- | | N/A | 2183 | TGPPGPQGLQ | | N/A | 3108 | GLPGTGGPPG |
| | collagen | | GLPG | | | | | | | | |
| 520 | type IV | 1276 | DPGE- | | N/A | 2184 | LPGMKGDPGE | | N/A | 3109 | ILGHVPGMLL |
| | collagen | | ILGH | | | | | | | | |
| | alpha-1 | | | | | | | | | | |
| | chain | | | | | | | | | | |
| 521 | type IV | 1277 | PPGP- | | N/A | 2185 | LPGSPGPPGP | | N/A | 3110 | PGDIVFRKGP |
| | collagen | | PGDI | | | | | | | | |
| | alpha-3 | | | | | | | | | | |
| | chain | | | | | | | | | | |
| 522 | type VII | 1278 | GRLV- | | N/A | 2186 | GPPGPPGRLV | | N/A | 3111 | DTGPGAREKG |
| | collagen | | DTGP | | | | | | | | E |
| | alpha-1 | | | | | | | | | | |
| | chain | | | | | | | | | | |
| 523 | fibrinogen | 1279 | ADSG- | 1683 | GGVR- | 2187 | VGTAWTADSG | 2593 | EGDFLAEGGGVR | 3112 | GPRVVERHQS |
| | alpha | | EGDF | | GPRV | | | | | | |
| | chain | | | | | | | | | | |
| 524 | fibrinogen | 1280 | AWTA- | 1684 | GGVR- | 2188 | LSVVGTAWTA | 2594 | DSGEGDFLAEGG | 3113 | GPRVVERHQS |
| | alpha | | DSGE | | GPRV | | | | GVR | | |
| | chain | | | | | | | | | | |
| 525 | elastin | 1281 | SPEA- | | N/A | 2189 | VPGVGISPEA | | N/A | 3114 | QAAAAAKAAK |
| | | | QAAA | | | | | | | | |
| 526 | C-reactive | 1282 | DMSR- | | N/A | 2190 | HAFGQTDMSR | | N/A | 3115 | KAFVFP |
| | protein | | KAFV | | | | | | | | |
| 527 | elastin | 1283 | GPGG- | | N/A | 2191 | GVAPGIGPGG | | N/A | 3116 | VAAAAKSAAK |
| | | | VAAA | | | | | | | | |
| 528 | type VI | 1284 | GAKG- | | N/A | 2192 | APRGVKGAKG | | N/A | 3117 | YRGPEGPQGP |
| | collagen | | YRGP | | | | | | | | |
| | alpha-1 | | | | | | | | | | |
| | chain | | | | | | | | | | |
| 529 | type V | 1285 | GPSG- | | N/A | 2193 | GPPGKRGPSG | | N/A | 3118 | HMGREGREGE |
| | collagen | | HMGR | | | | | | | | |
| | alpha-1 | | | | | | | | | | |
| | chain | | | | | | | | | | |
| 530 | complement | 1286 | STGR- | 1685 | RQIR- | 2194 | LNVTLSSTGR | 2595 | NGFKSHALQLNN | 3119 | GLEEELQFSL |
| | C4-A OR | | NGFK | | GLEE | | | | RQIR | | |
| | complement | | | | | | | | | | |
| | C4-B | | | | | | | | | | |
| 531 | complement | 1287 | LPSR- | 1686 | SLLR- | 2195 | LDVSLQLPSR | 2596 | SSKITHRIHWES | 3120 | SEETKENEGF |
| | C3 | | SSKI | | SEET | | | | ASLLR | | |
| 532 | fibrinogen | 1288 | FESK- | 1687 | RPVR- | 2196 | NRGDSTFESK | 2597 | SYKMADEAGSEA | 3121 | DCDDVLQTHP |
| | alpha | | SYKM | | DCDD | | | | DHEGTHSTKRGH | | |
| | chain | | | | | | | | AKSRPVR | | |
| 533 | nidogen-1 | 1289 | HERE- | | N/A | 2197 | VEKTRCQHER | | N/A | 3122 | HILGAAGATD |
| | | | HILG | | | | E | | | | |
| 534 | type VI | 1290 | GNRG- | | N/A | 2198 | GPKGGIGNRG | | N/A | 3123 | PRGETGDDGR |
| | collagen | | PRGE | | | | | | | | |
| | alpha-3 | | | | | | | | | | |
| | chain | | | | | | | | | | |

*The (putative) scissile bond of each cleavage sequence listed in Table A, cleavage sequence 1 and cleavage sequence 2 (if present) in each reporter polypeptide, is indicated by a hyphen (-).
“N/A” indicates that the amino acid sequence of the corresponding cleavage sequence is not, or cannot be, specified in the instance.

In some embodiments of the compositions (such as the therapeutic agents, or activatable therapeutic agents described hereinabove) or methods described herein, the mammalian protease (for cleavage of the release segment (RS), or the first release segment (RS1), or the second release segment (RS2)) can be a serine protease, a cysteine protease, an aspartate protease, a threonine protease, or a metalloproteinase. The mammalian protease (for cleavage of the release segment (RS), or the first release segment (RS1), or the second release segment (RS2)) can be selected from the group consisting of disintegrin and metalloproteinase domain-containing protein 10 (ADAM10), disintegrin and metalloproteinase domain-containing protein 12 (ADAM12), disintegrin and metalloproteinase domain-containing protein 15 (ADAM15), disintegrin and metalloproteinase domain-containing protein 17 (ADAM17), disintegrin and metalloproteinase domain-containing protein 9 (ADAM9), disintegrin and metalloproteinase with thrombospondin motifs 5 (ADAMTS5), Cathepsin B, Cathepsin D, Cathepsin E, Cathepsin K, cathepsin L, cathepsin S, Fibroblast activation protein alpha, Hepsin, kallikrein-2, kallikrein-4, kallikrein-3, Prostate-specific antigen (PSA), kallikrein-13, Legumain, matrix metallopeptidase 1 (MMP-1), matrix metallopeptidase 10 (MMP-10), matrix metallopeptidase 11 (MMP-11), matrix metallopeptidase 12 (MMP-12), matrix metallopeptidase 13 (MMP-13), matrix metallopeptidase 14 (MMP-14), matrix metallopeptidase 16 (MMP-16), matrix metallopeptidase 2 (MMP-2), matrix metallopeptidase 3 (MMP-3), matrix metallopeptidase 7 (MMP-7), matrix metallopeptidase 8 (MMP-8), matrix metallopeptidase 9 (MMP-9), matrix metallopeptidase 4 (MMP-4), matrix metallopeptidase 5 (MMP-5), matrix metallopeptidase 6 (MMP-6), matrix metallopeptidase 15 (MMP-15), neutrophil elastase, protease activated receptor 2 (PAR2), plasmin, prostasin, PSMA-FOLH1, membrane type serine protease 1 (MT-SP1), matriptase, and u-plasminogen. The mammalian protease (for cleavage of the release segment (RS), or the first release segment (RS1), or the second release segment (RS2)) can be selected from the group consisting of matrix metallopeptidase 1 (MMP1), matrix metallopeptidase 2 (MMP2), matrix metallopeptidase 7 (MMP1), matrix metallopeptidase 9 (MMP9), matrix metallopeptidase 11 (MMP11), matrix metallopeptidase 14 (MMP14), urokinase-type plasminogen activator (uPA), legumain, and matriptase. The mammalian protease can be preferentially expressed or activated in the target tissue or cell.

In some embodiments of the compositions (such as the therapeutic agents, or activatable therapeutic agents described hereinabove) or methods described herein, the target tissue or cell can be characterized by an increased amount or activity of a mammalian protease (such as one described herein) in proximity to the target tissue or cell as compared to a non-target tissue or cell in a subject. The target tissue or cell can be characterized by a presence, in proximity thereto, of at least (about) 10% more, at least (about) 20% more, at least (about) 30% more, at least (about) 40% more, at least (about) 50% more, at least (about) 60% more, at least (about) 70% more, at least (about) 80% more, at least (about) 90% more, at least (about) 100% more, or at least (about) 200% more amount of the mammalian protease as compared to a non-target tissue or cell in the subject. The target tissue or cell can be characterized by an activity, in proximity thereto, of the mammalian protease of at least (about) 10% higher, at least (about) 20% higher, at least (about) 30% higher, at least (about) 40% higher, at least (about) 50% higher, at least (about) 60% higher, at least (about) 70% higher, at least (about) 80% higher, at least (about) 90% higher, at least (about) 100% higher, or at least (about) 200% higher as compared to a non-target tissue or cell in the subject. The target tissue or cell can produce or can be co-localized with the mammalian protease (such as one described herein). The target tissue or cell can be a tumor.

In some embodiments, the compositions of this disclosure (such as activatable therapeutic agents) are designed with considerations of the location of the target tissue protease as well as the presence of the same protease in healthy tissues not intended to be targeted, but a greater presence of the ligand in unhealthy target tissue, in order to provide a wide therapeutic window. A “therapeutic window” refers to the largest difference between the minimal effective dose and the maximal tolerated dose for a given therapeutic composition. To help achieve a wide therapeutic window, the binding domains of the compositions are shielded by the proximity of the masking moiety (e.g., XTEN) such that the binding affinity of the intact composition for one or both of the ligands is reduced compared to the composition that has been cleaved by a mammalian protease, thereby releasing the biologically active moiety from the shielding effects of the masking moiety.

### Nucleic Acids, Expression Vectors, Host Cells

Provided herein, in some embodiments, is an isolated nucleic acid comprising: (a) a polynucleotide encoding a recombinant polypeptide as described herein; or (b) a reverse complement of the polynucleotide of (a).

Provided herein, in some embodiments, is an expression vector comprising a polynucleotide sequence as described herein and a recombinant regulatory sequence operably linked to the polynucleotide sequence.

Provided herein, in some embodiments, is an isolated host cell, comprising an expression vector as described herein. The isolated host cell can be a prokaryote. The isolated host cell can be E. coli. The isolated host cell can be mammalian cell(s).

### Pharmaceutical Compositions

Provided herein, in some embodiments, is a pharmaceutical composition comprising a therapeutic agent (such as described hereinabove or described anywhere else herein) and one or more pharmaceutically suitable excipients. The pharmaceutical composition can be formulated for oral, intradermal, subcutaneous, intravenous, intra-arterial, intraabdominal, intraperitoneal, intrathecal, or intramuscular administration. The pharmaceutical composition can be in a liquid form or frozen form. The pharmaceutical composition can be in a pre-filled syringe for a single injection. The pharmaceutical composition can be formulated as a lyophilized powder to be reconstituted prior to administration.

### Kits

Provided herein, in some embodiments, is a kit comprising a pharmaceutical composition described herein (or a therapeutic agent described herein), a container, and a label or package insert on or associated with the container.

### Methods

### Methods for Assessing a Likelihood of a Response to Therapeutic Agent(s)

Provided herein, in some embodiments, is a method for assessing a likelihood of a subject being responsive to a therapeutic agent that is activatable by a mammalian protease expressed in the subject, the method comprising:
(a) determining, in a biological sample from the subject, a presence or an amount of
   (i) a polypeptide comprising at least four, at least five, at least six, at least seven, at least eight, at least nine, or at least ten consecutive amino acid residues shown in a sequence set forth in Column V of Table A (or a subset thereof); or
   (ii) a polypeptide comprising at least four, at least five, at least six, at least seven, at least eight, at least nine, or at least ten consecutive amino acids shown in a sequence set forth in Column IV of Table A (or a subset thereof); or
   (iii) a polypeptide comprising at least four, at least five, at least six, at least seven, at least eight, at least nine, or at least ten consecutive amino acids shown in a sequence set forth in Column VI of Table A (or a subset thereof); and
(b) designating the subject as being likely to respond to the therapeutic agent when the polypeptide of (i), (ii) or (iii) is present and/or if its amount exceeds a threshold.

In some embodiments of the method for assessing the likelihood of the subject being responsive to the therapeutic agent, the therapeutic agent can comprise a peptide substrate susceptible to cleavage by the mammalian protease (e.g., at a scissile bond). The peptide substrate can be susceptible to cleavage by the mammalian protease at a scissile bond. The polypeptide of (i), (ii), or (iii) can comprise a portion (e.g., containing at least four, at least five, at least six, at least seven, at least eight, at least nine, at least ten, at least eleven, at least twelve, at least thirteen, at least fourteen, or at least fifteen consecutive amino acid residues) of the peptide substrate that is either N-terminal or C-terminal side of the scissile bond. The portion (e.g., containing at least four, at least five, at least six, at least seven, at least eight, at least nine, at least ten, at least eleven, at least twelve, at least thirteen, at least fourteen, or at least fifteen consecutive amino acid residues) of the peptide substrate can be either immediately N-terminal or immediately C-terminal of the scissile bond. The polypeptide of (i) can comprise at least four, at least five, at least six, at least seven, at least eight, at least nine, or at least ten consecutive amino acid residues shown in a sequence set forth in Column V of Table A (or a subset thereof). The polypeptide of (i) can comprise a sequence set forth in Column V of Table A (or a subset thereof). The polypeptide of (ii) can comprise at least four, at least five, at least six, at least seven, at least eight, at least nine, or at least ten consecutive amino acids shown in a sequence set forth in Column IV of Table A (or a subset thereof). The polypeptide of (ii) can comprise a sequence set forth in Column IV of Table A (or a subset thereof). The polypeptide of (iii) can comprise at least four, at least five, at least six, at least seven, at least eight, at least nine, or at least ten consecutive amino acids shown in a sequence set forth in Column VI of Table A (or a subset thereof). The polypeptide of (iii) can comprise a sequence set forth in Column VI of Table A (or a subset thereof). In some embodiments of the method for assessing the likelihood, (a) comprises determining the presence or the amount of any two of (i)-(iii). In some embodiments of the method for assessing the likelihood, (a) comprises determining the presence or the amount of all three of (i)-(iii). Additionally or alternatively, the subject designated, by the method described herein in the section entitled “METHODS FOR ASSESSING A LIKELIHOOD OF A RESPONSE TO THERAPEUTIC AGENT(S),” as being likely to respond to the activatable therapeutic agent (such as one described herein) can be one with an expression profile of biomarker(s) such that, upon administering an activatable therapeutic agent (such as one described herein) to the subject, the activatable therapeutic agent is more likely than not to be cleaved at or near the target tissue(s) or cell(s) (such as described herein in the “Target Tissues or Cells” section), e.g., by mammalian protease(s), thereby activating the therapeutic agent. In some embodiments of the method for assessing the likelihood, the threshold can be zero or nominal. The peptide substrate can be any peptide substrate described hereinabove in the RELEASE SEGMENTS section or described anywhere else herein. The activatable therapeutic agent can be any therapeutic agent (or any activatable therapeutic agent, or any non-natural, activatable therapeutic agent) as described hereinabove in the THERAPEUTIC AGENTS section or described anywhere else herein. The mammalian protease can be any mammalian protease as described hereinabove in the TARGET TISSUES OR CELLS section or described anywhere else herein. The target tissue or cell can be any one described hereinabove in the TARGET TISSUES OR CELLS section or described anywhere else herein. The target tissue or cell can be a tumor.

In some embodiments of the method for assessing the likelihood, the biological sample can be selected from serum, plasma, blood, spinal fluid, semen, and saliva. The biological sample can comprise a serum or plasma sample. The biological sample can comprise a serum sample. The biological sample can comprise a plasma sample. The biological sample can comprise a blood sample. The biological sample can comprise a spinal fluid sample. The biological sample can comprise a semen sample. The biological sample can comprise a saliva sample.

In some embodiments of the method for assessing the likelihood, the subject can be suffering from, or can be suspected of suffering from, a disease or condition characterized by an increased expression or activity of the mammalian protease in proximity to a target tissue or cell (such as one described hereinabove in the TARGET TISSUES OR CELLS section or described anywhere else herein) as compared to a corresponding non-target tissue or cell in the subject. The subject can be selected from mouse, rat, monkey, and human. The subject can be a human. In some embodiments, the disease or condition can be a cancer or an inflammatory or autoimmune disease. In some embodiments, the disease or condition can be a cancer. The cancer can be selected from the group consisting of carcinoma, Hodgkin's lymphoma, and non-Hodgkin's lymphoma, diffuse large B cell lymphoma, follicular lymphoma, mantle cell lymphoma, blastoma, breast cancer, ER/PR+ breast cancer, Her2+ breast cancer, triple-negative breast cancer, colon cancer, colon cancer with malignant ascites, mucinous tumors, prostate cancer, head and neck cancer, skin cancer, melanoma, genito-urinary tract cancer, ovarian cancer, ovarian cancer with malignant ascites, peritoneal carcinomatosis, uterine serous carcinoma, endometrial cancer, cervix cancer, colorectal, uterine cancer, mesothelioma in the peritoneum, kidney cancer, Wilm's tumor, lung cancer, small-cell lung cancer, non-small cell lung cancer, gastric cancer, stomach cancer, small intestine cancer, liver cancer, hepatocarcinoma, hepatoblastoma, liposarcoma, pancreatic cancer, gall bladder cancer, cancers of the bile duct, esophageal cancer, salivary gland carcinoma, thyroid cancer, epithelial cancer, arrhenoblastoma, adenocarcinoma, sarcoma, and B-cell derived chronic lymphatic leukemia. In some embodiments, the disease or condition can be an inflammatory or autoimmune disease. The inflammatory or autoimmune disease can be selected from the group consisting of ankylosing spondylitis (AS), arthritis (for example, and not limited to, rheumatoid arthritis (RA), juvenile idiopathic arthritis (JIA), osteoarthritis (OA), psoriatic arthritis (PsA), gout, chronic arthritis), chagas disease, chronic obstructive pulmonary disease (COPD), dermatomyositis, type 1 diabetes, endometriosis, Goodpasture syndrome, Graves' disease, Guillain-Barre syndrome (GB S), Hashimoto's disease, suppurative scab, Kawasaki disease, IgA nephropathy, idiopathic thrombocytopenic purpura, inflammatory bowel disease (IBD) (for example, and not limited to, Crohn's disease (CD), clonal disease, ulcerative colitis, collagen colitis, lymphocytic colitis, ischemic colitis, empty colitis, Behcet's syndrome, infectious colitis, indeterminate colitis, interstitial Cystitis), lupus (for example, and not limited to, systemic lupus erythematosus, discoid lupus, subacute cutaneous lupus erythematosus, cutaneous lupus erythematosus (such as chilblain lupus erythematosus), drug-induced lupus, neonatal lupus, lupus nephritis), mixed connective tissue disease, morphea, multiple sclerosis (MS), severe muscle Force disorder, narcolepsy, neuromuscular angina, pemphigus vulgaris, pernicious anemia, psoriasis, psoriatic arthritis, polymyositis, primary biliary cirrhosis, relapsing polychondritis, schizophrenia, scleroderma, Sjogren's syndrome, systemic stiffness syndrome, temporal arteritis (also known as giant cell arteritis), vasculitis, vitiligo, Wegener's granulomatosis, transplant rejection-associated immune reaction(s) (for example, and not limited to, renal transplant rejection, lung transplant rejection, liver transplant rejection), psoriasis, Wiskott-Aldrich syndrome, autoimmune lymphoproliferative syndrome, myasthenia gravis, inflammatory chronic rhinosinusitis, colitis, celiac disease, Barrett's esophagus, inflammatory gastritis, autoimmune nephritis, autoimmune hepatitis, autoimmune carditis, autoimmune encephalitis, autoimmune mediated hematological disease, asthma, atopic dermatitis, atopy, allergy, allergic rhinitis, scleroderma, bronchitis, pericarditis, the inflammatory disease is, Alzheimer's disease, Parkinson's disease, amyotrophic lateral sclerosis, inflammatory lung disease, inflammatory skin disease, atherosclerosis, myocardial infarction, stroke, gram-positive shock, gram-negative shock, sepsis, septic shock, hemorrhagic shock, anaphylactic shock, systemic inflammatory response syndrome. Additionally or alternatively, the subject designated, by the method described herein in the section entitled “METHODS FOR ASSESSING A LIKELIHOOD OF A RESPONSE TO THERAPEUTIC AGENT(S),” as being likely to respond to the activatable therapeutic agent (such as one described herein) can be one with an expression profile of biomarker(s) such that, upon administering an activatable therapeutic agent (such as one described herein) to the subject, the activatable therapeutic agent is more likely than not to be cleaved at or near the target tissue(s) or cell(s) (such as described herein in the “Target Tissues or Cells” section), e.g., by mammalian protease(s), thereby activating the therapeutic agent. In some embodiments, the method for assessing the likelihood can further comprise transmitting the designation to a healthcare provider and/or the subject. In some embodiments, the method for assessing the likelihood can further comprise, subsequent to (b), contacting the therapeutic agent with the mammalian protease. In some embodiments, the method for assessing the likelihood can further comprise, subsequent to (b), administering to the subject an effective amount of the therapeutic agent based on the designation of step (b). In some embodiments of the method for assessing the likelihood, (a) can comprise detecting the polypeptide of (i), (ii) or (iii) in an immunoassay. The immunoassay can utilize an antibody that specifically binds to the polypeptide of (i), (ii) or (iii), or an epitope thereof. In some embodiments of the method for assessing the likelihood, (a) can comprise detecting the polypeptide of (i), (ii) or (iii) by using a mass spectrometer (MS) (including but not limited to LC-MS, LC-MS/MS, etc.).

### Methods for Preparing Therapeutic Agent(s)

Provided herein, in some embodiments, is a method for preparing an activatable therapeutic agent, the method comprising:
(a) culturing a host cell comprising a nucleic acid construct that encodes a recombinant polypeptide under conditions sufficient to express the recombinant polypeptide in the host cell, wherein the recombinant polypeptide comprises a biologically active polypeptide (BP), a release segment (RS), and a masking moiety (MM), wherein:
   the RS comprises a peptide substrate susceptible for cleavage by a mammalian protease at a scissile bond, wherein the peptide substrate comprises an amino acid sequence having at least 80% sequence identity to a sequence set forth in Column II or III of Table A (or a subset thereof) and/or the group set forth in Tables 1(a)-1(j); and
   the recombinant polypeptide has a structural arrangement from N-terminus to C-terminus of BP-RS-MM or MM-RS-BP; and
(b) recovering the activatable therapeutic agent comprising the recombinant polypeptide.

In some embodiments of the method for preparing the activatable therapeutic agent, the release segment (RS) can be a first release segment (RS1), the peptide substrate can be a first peptide substrate, the scissile bond can be a first scissile bond, the masking moiety (MM) can be a first masking moiety (MM1), and the recombinant polypeptide can further comprise a second release segment (RS2), and a second masking moiety (MM2), where:
the RS2 comprises a second peptide substrate susceptible for cleavage by a mammalian protease at a second scissile bond, where the second peptide substrate can comprise an amino acid sequence having at least 80% sequence identity to a sequence set forth in Column II or III of Table A (or a subset thereof) and/or the group set forth in Tables 1(a)-1(j); and
the recombinant polypeptide can have a structural arrangement from N-terminus to C-terminus of MM1-RS1-BP-RS2-MM2, MM1-RS2-BP-RS1-MM2, MM2-RS1-BP-RS2-MM1, or MM2-RS2-BP-RS1-MM1.

In some embodiments of the method for preparing the activatable therapeutic agent, the masking moiety (MM) can comprise an extended recombinant polypeptide (XTEN) (such as one described hereinabove in the MASKING MOIETIES section or described anywhere else herein). In some embodiments of the method for preparing the activatable therapeutic agent, where the activatable therapeutic agent comprises a first masking moiety (MM1) and a second masking moiety (MM2), one of the MM1 and the MM2 can be a first extended recombinant polypeptide (XTEN1) (such as one described hereinabove in the MASKING MOIETIES section or described anywhere else herein). The other one of the MM1 and the MM2 can comprise a second extended recombinant polypeptide (XTEN2) (such as one described hereinabove in the MASKING MOIETIES section or described anywhere else herein).

In some embodiments of the method for preparing the activatable therapeutic agent, the recombinant polypeptide can be anyone described herein. The masking moiety (MM), when linked to the recombinant polypeptide, can interfere with an interaction of the biologically active peptide (BP) to a target tissue or cell such that a dissociation constant (K_{d}) of the BP of the recombinant polypeptide with a target cell marker borne by the target tissue or cell can be greater, when the recombinant polypeptide is in an uncleaved state, compared to a dissociation constant (K_{d}) of a corresponding biologically active peptide released from the recombinant polypeptide. The first masking moiety (MM1) and the second masking moiety (MM2), when both linked in the recombinant polypeptide, can (each independently, individually or collectively) interfere with an interaction of the biologically active peptide (BP) to a target tissue or cell such that a dissociation constant (K_{d}) of the BP of the recombinant polypeptide with a target cell marker borne by the target tissue or cell can be greater, when the recombinant polypeptide is in an uncleaved state, compared to a dissociation constant (K_{d}) of a corresponding biologically active peptide, when one or both of the first release segment (RS1) and the second release segment (RS2) is/are cleaved. The dissociation constant (Kd) can be measured in an in vitro assay under equivalent molar concentrations. The in vitro assay can be selected from cell membrane integrity assay, mixed cell culture assay, cell-based competitive binding assay, FACS based propidium Iodide assay, trypan Blue influx assay, photometric enzyme release assay, radiometric 51Cr release assay, fluorometric Europium release assay, CalceinAM release assay, photometric MTT assay, XTT assay, WST-1 assay, alamar blue assay, radiometric 3H-Thd incorporation assay, clonogenic assay measuring cell division activity, fluorometric rhodamine123 assay measuring mitochondrial transmembrane gradient, apoptosis assay monitored by FACS-based phosphatidylserine exposure, ELISA-based TUNEL test assay, sandwich ELISA, caspase activity assay, cell-based LDH release assay, reporter gene activity assay, and cell morphology assay, or any combination thereof.

Methods for Treating Subjects with Therapeutic Agent(s)

Provided herein, in some embodiments, is a method for treating a subject with an activatable therapeutic agent, the method comprising:
(a) identifying the subject as having a likelihood of a response to the activatable therapeutic agent based on identification of a peptide biomarker in a biological sample from the subject, which activatable therapeutic agent comprises a peptide substrate susceptible to cleavage by a mammalian protease at a scissile bond; and
(b) administering the activatable therapeutic agent to the subject based on the identification of the subject in (a);
wherein the peptide biomarker comprises a portion identical to at least four consecutive amino acid residues of the peptide substrate that is either N-terminal or C-terminal of the scissile bond.

In some embodiments described in the immediately preceding paragraph, the peptide substrate can be any peptide substrate described hereinabove in the RELEASE SEGMENTS section or described anywhere else herein. The activatable therapeutic agent can be any therapeutic agent (or any activatable therapeutic agent, or any non-natural, activatable therapeutic agent) as described hereinabove in the THERAPEUTIC AGENTS section or described anywhere else herein. The mammalian protease can be any mammalian protease as described hereinabove in the TARGET TISSUES OR CELLS section or described anywhere else herein. The peptide biomarker can be any peptide biomarker as described hereinabove in the TARGET TISSUES OR CELLS section (such as those set forth in Table A) or described anywhere else herein. The likelihood of the response can be determined by a method as described hereinabove in the METHODS FOR ASSESSING A LIKELIHOOD OF A RESPONSE TO THERAPEUTIC AGENT(S) section or described anywhere else herein. The portion containing at least four consecutive amino acid residues can contain at least five, at least six, at least seven, at least eight, at least nine, at least ten, at least eleven, at least twelve, at least thirteen, at least fourteen, or at least fifteen consecutive amino acid residues of the peptide substrate that is either N-terminal or C-terminal of the scissile bond. The portion containing at least four (e.g., at least five, at least six, at least seven, at least eight, at least nine, at least ten, at least eleven, at least twelve, at least thirteen, at least fourteen, or at least fifteen) consecutive amino acid residues of the peptide substrate can be either immediately N-terminal or immediately C-terminal of the scissile bond. Additionally or alternatively, the subject designated, by the method described herein in the section entitled “METHODS FOR ASSESSING A LIKELIHOOD OF A RESPONSE TO THERAPEUTIC AGENT(S),” as being likely to respond to the activatable therapeutic agent (such as one described herein) can be one with an expression profile of biomarker(s) such that, upon administering an activatable therapeutic agent (such as one described herein) to the subject, the activatable therapeutic agent is more likely than not to be cleaved at or near the target tissue(s) or cell(s) (such as described herein in the “Target Tissues or Cells” section), e.g., by mammalian protease(s), thereby activating the therapeutic agent. In some embodiments, the peptide biomarker can be derived from a reporter polypeptide (such as described herein). In some embodiments, the peptide biomarker can have an amino acid sequence that is identical to a sequence of a reporter polypeptide. The reporter polypeptide can comprise a sequence set forth in Columns II-VI of Table A (or a subset thereof). In some embodiments, the peptide substrate can comprise an amino acid sequence having at most three, at most two, or at most one amino acid substitution(s) with respect to a sequence set forth in Column II or III of Table A (or a subset thereof). In some embodiments, none of the amino acid substitution can be at a position corresponding to an amino acid residue immediately adjacent to a corresponding scissile bond as indicated in Table A. In some embodiments, the peptide substrate can comprise an amino acid sequence set forth in Column II or III of Table A (or a subset thereof). In some embodiments, the peptide substrate can comprise an amino acid sequence having at most three, at most two or at most one amino acid substitution(s) with respect to a sequence set forth in Table 1(j). In some embodiments, none of the amino acid substitution can be at a position corresponding to an amino acid residue immediately adjacent to a corresponding scissile bond set forth in Table 1(j). In some embodiments, the peptide substrate can comprise an amino acid sequence set forth in Table 1(j).

Provided herein, in some embodiments, is a method for treating a subject in need of a therapeutic agent that is activatable by a mammalian protease expressed in the subject, the method comprising:
administering an effective amount of the therapeutic agent to the subject, wherein the subject has been shown to express in a biological sample from the subject:
   (i) a polypeptide comprising at least four, at least five, at least six, at least seven, at least eight, at least nine, or at least ten consecutive amino acid residues shown in a sequence set forth in Column V of Table A (or a subset thereof); or
   (ii) a polypeptide comprising at least four, at least five, at least six, at least seven, at least eight, at least nine, or at least ten consecutive amino acids shown in a sequence set forth in Column IV of Table A (or a subset thereof); or
   (iii) a polypeptide comprising at least four, at least five, at least six, at least seven, at least eight, at least nine, or at least ten consecutive amino acids shown in a sequence set forth in Column VI of Table A (or a subset thereof); or
   (iv) expression level of polypeptide (i), (ii) or (iii) exceeds a threshold.

In some embodiments described in the immediately preceding paragraph, the threshold can be zero or nominal. The peptide substrate can be any peptide substrate described hereinabove in the RELEASE SEGMENTS section or described anywhere else herein. The activatable therapeutic agent can be any therapeutic agent (or any activatable therapeutic agent, or any non-natural, activatable therapeutic agent) as described hereinabove in the THERAPEUTIC AGENTS section or described anywhere else herein. The mammalian protease can be any mammalian protease as described hereinabove in the TARGET TISSUES OR CELLS section or described anywhere else herein. The likelihood of the response can be determined by a method described hereinabove in the METHODS FOR ASSESSING A LIKELIHOOD OF A RESPONSE TO THERAPEUTIC AGENT(S) section or described anywhere else herein. The polypeptide of (i) can comprise at least four, at least five, at least six, at least seven, at least eight, at least nine, or at least ten consecutive amino acid residues shown in a sequence set forth in Column V of Table A (or a subset thereof). The polypeptide of (i) can comprise a sequence set forth in Column V of Table A (or a subset thereof). The polypeptide of (ii) can comprise at least four, at least five, at least six, at least seven, at least eight, at least nine, or at least ten consecutive amino acids shown in a sequence set forth in Column IV of Table A (or a subset thereof). The polypeptide of (ii) can comprise a sequence set forth in Column IV of Table A (or a subset thereof). The polypeptide of (iii) can comprise at least four, at least five, at least six, at least seven, at least eight, at least nine, or at least ten consecutive amino acids shown in a sequence set forth in Column VI of Table A (or a subset thereof). The polypeptide of (iii) can comprise a sequence set forth in Column VI of Table A (or a subset thereof). The therapeutic agent can comprise a peptide substrate susceptible to cleavage by the mammalian protease (e.g., at a scissile bond). The peptide substrate can be susceptible to cleavage by the mammalian protease at a scissile bond, and the polypeptide of (i), (ii), or (iii) can comprise a portion (e.g., containing at least four, at least five, at least six, at least seven, at least eight, at least nine, at least ten, at least eleven, at least twelve, at least thirteen, at least fourteen, or at least fifteen consecutive amino acid residues) of the peptide substrate that is either N-terminal or C-terminal of the scissile bond. The portion (e.g., containing at least four, at least five, at least six, at least seven, at least eight, at least nine, at least ten, at least eleven, at least twelve, at least thirteen, at least fourteen, or at least fifteen consecutive amino acid residues) of the peptide substrate can be either immediately N-terminal or immediately C-terminal of the scissile bond. In some embodiments, the subject has been shown to express in the biological sample any two of (i)-(iii). In some embodiments, the subject has been shown to express in the biological sample all three of (i)-(iii).

In some embodiments of the method described herein this METHODS FOR TREATING SUBJECTS WITH THERAPEUTIC AGENT(S) section, the biological sample can be selected from serum, plasma, blood, spinal fluid, semen, and saliva. The biological sample can comprise a serum or plasma sample. The biological sample can comprise a serum sample. The biological sample can comprise a plasma sample. The biological sample can comprise a blood sample. The biological sample can comprise a spinal fluid sample. The biological sample can comprise a semen sample. The biological sample can comprise a saliva sample.

In some embodiments of the method described herein this METHODS FOR TREATING SUBJECTS WITH THERAPEUTIC AGENT(S) section, the subject can be suffering from, or can be suspected of suffering from, a disease or condition characterized by an increased expression or activity of the mammalian protease in proximity to a target tissue or cell (such as one described hereinabove in the TARGET TISSUES OR CELLS section or described anywhere else herein) as compared to a corresponding non-target tissue or cell in the subject. The subject can be selected from mouse, rat, monkey, and human. The subject can be a human. The subject can be determined to have a likelihood of a response to the therapeutic agent or the pharmaceutical composition. The likelihood of the response can be 50% or higher. The likelihood of the response can be determined by a method as described herein (such as one described hereinabove in the METHODS FOR ASSESSING A LIKELIHOOD OF A RESPONSE TO THERAPEUTIC AGENT(S) section). In some embodiments, the disease or condition can be a cancer or an inflammatory or autoimmune disease. In some embodiments, the disease or condition can be a cancer. The cancer can be selected from the group consisting of carcinoma, Hodgkin's lymphoma, and non-Hodgkin's lymphoma, diffuse large B cell lymphoma, follicular lymphoma, mantle cell lymphoma, blastoma, breast cancer, ER/PR+ breast cancer, Her2+ breast cancer, triple-negative breast cancer, colon cancer, colon cancer with malignant ascites, mucinous tumors, prostate cancer, head and neck cancer, skin cancer, melanoma, genito-urinary tract cancer, ovarian cancer, ovarian cancer with malignant ascites, peritoneal carcinomatosis, uterine serous carcinoma, endometrial cancer, cervix cancer, colorectal, uterine cancer, mesothelioma in the peritoneum, kidney cancer, Wilm's tumor, lung cancer, small-cell lung cancer, non-small cell lung cancer, gastric cancer, stomach cancer, small intestine cancer, liver cancer, hepatocarcinoma, hepatoblastoma, liposarcoma, pancreatic cancer, gall bladder cancer, cancers of the bile duct, esophageal cancer, salivary gland carcinoma, thyroid cancer, epithelial cancer, arrhenoblastoma, adenocarcinoma, sarcoma, and B-cell derived chronic lymphatic leukemia. In some embodiments, the disease or condition can be an inflammatory or autoimmune disease. The inflammatory or autoimmune disease can be selected from the group consisting of ankylosing spondylitis (AS), arthritis (for example, and not limited to, rheumatoid arthritis (RA), juvenile idiopathic arthritis (JIA), osteoarthritis (OA), psoriatic arthritis (PsA), gout, chronic arthritis), chagas disease, chronic obstructive pulmonary disease (COPD), dermatomyositis, type 1 diabetes, endometriosis, Goodpasture syndrome, Graves' disease, Guillain-Barre syndrome (GBS), Hashimoto's disease, suppurative scab, Kawasaki disease, IgA nephropathy, idiopathic thrombocytopenic purpura, inflammatory bowel disease (IBD) (for example, and not limited to, Crohn's disease (CD), clonal disease, ulcerative colitis, collagen colitis, lymphocytic colitis, ischemic colitis, empty colitis, Behcet's syndrome, infectious colitis, indeterminate colitis, interstitial Cystitis), lupus (for example, and not limited to, systemic lupus erythematosus, discoid lupus, subacute cutaneous lupus erythematosus, cutaneous lupus erythematosus (such as chilblain lupus erythematosus), drug-induced lupus, neonatal lupus, lupus nephritis), mixed connective tissue disease, morphea, multiple sclerosis (MS), severe muscle Force disorder, narcolepsy, neuromuscular angina, pemphigus vulgaris, pernicious anemia, psoriasis, psoriatic arthritis, polymyositis, primary biliary cirrhosis, relapsing polychondritis, schizophrenia, scleroderma, Sjogren's syndrome, systemic stiffness syndrome, temporal arteritis (also known as giant cell arteritis), vasculitis, vitiligo, Wegener's granulomatosis, transplant rejection-associated immune reaction(s) (for example, and not limited to, renal transplant rejection, lung transplant rejection, liver transplant rejection), psoriasis, Wiskott-Aldrich syndrome, autoimmune lymphoproliferative syndrome, myasthenia gravis, inflammatory chronic rhinosinusitis, colitis, celiac disease, Barrett's esophagus, inflammatory gastritis, autoimmune nephritis, autoimmune hepatitis, autoimmune carditis, autoimmune encephalitis, autoimmune mediated hematological disease, asthma, atopic dermatitis, atopy, allergy, allergic rhinitis, scleroderma, bronchitis, pericarditis, the inflammatory disease is, Alzheimer's disease, Parkinson's disease, amyotrophic lateral sclerosis, inflammatory lung disease, inflammatory skin disease, atherosclerosis, myocardial infarction, stroke, gram-positive shock, gram-negative shock, sepsis, septic shock, hemorrhagic shock, anaphylactic shock, systemic inflammatory response syndrome. Additionally or alternatively, the subject designated, by the method described herein in the section entitled “METHODS FOR ASSESSING A LIKELIHOOD OF A RESPONSE TO THERAPEUTIC AGENT(S),” as being likely to respond to the activatable therapeutic agent (such as one described herein) can be one with an expression profile of biomarker(s) such that, upon administering an activatable therapeutic agent (such as one described herein) to the subject, the activatable therapeutic agent is more likely than not to be cleaved at or near the target tissue(s) or cell(s) (such as described herein in the “Target Tissues or Cells” section), e.g., by mammalian protease(s), thereby activating the therapeutic agent.

### Methods and Uses of Therapeutic Agent(s)

Provided herein, in some embodiments, is a method for treating a disease or condition in a subject, comprising administering to the subject in need thereof one or more therapeutically effective doses of a therapeutic agent (such as one described herein) or a pharmaceutical composition (such as one described herein). The subject can be selected from mouse, rat, monkey, and human. The subject can be a human. The subject can be determined to have a likelihood of a response to the therapeutic agent or the pharmaceutical composition. The likelihood of the response can be 50% or higher. The likelihood of the response can be determined by a method as described herein (such as one described hereinabove in the METHODS FOR ASSESSING A LIKELIHOOD OF A RESPONSE TO THERAPEUTIC AGENT(S) section). In some embodiments, the disease or condition can be a cancer or an inflammatory or autoimmune disease. In some embodiments, the disease or condition can be a cancer. The cancer can be selected from the group consisting of carcinoma, Hodgkin's lymphoma, and non-Hodgkin's lymphoma, diffuse large B cell lymphoma, follicular lymphoma, mantle cell lymphoma, blastoma, breast cancer, ER/PR+ breast cancer, Her2+ breast cancer, triple-negative breast cancer, colon cancer, colon cancer with malignant ascites, mucinous tumors, prostate cancer, head and neck cancer, skin cancer, melanoma, genito-urinary tract cancer, ovarian cancer, ovarian cancer with malignant ascites, peritoneal carcinomatosis, uterine serous carcinoma, endometrial cancer, cervix cancer, colorectal, uterine cancer, mesothelioma in the peritoneum, kidney cancer, Wilm's tumor, lung cancer, small-cell lung cancer, non-small cell lung cancer, gastric cancer, stomach cancer, small intestine cancer, liver cancer, hepatocarcinoma, hepatoblastoma, liposarcoma, pancreatic cancer, gall bladder cancer, cancers of the bile duct, esophageal cancer, salivary gland carcinoma, thyroid cancer, epithelial cancer, arrhenoblastoma, adenocarcinoma, sarcoma, and B-cell derived chronic lymphatic leukemia. In some embodiments, the disease or condition can be an inflammatory or autoimmune disease. The inflammatory or autoimmune disease can be selected from the group consisting of ankylosing spondylitis (AS), arthritis (for example, and not limited to, rheumatoid arthritis (RA), juvenile idiopathic arthritis (JIA), osteoarthritis (OA), psoriatic arthritis (PsA), gout, chronic arthritis), chagas disease, chronic obstructive pulmonary disease (COPD), dermatomyositis, type 1 diabetes, endometriosis, Goodpasture syndrome, Graves' disease, Guillain-Barre syndrome (GBS), Hashimoto's disease, suppurative scab, Kawasaki disease, IgA nephropathy, idiopathic thrombocytopenic purpura, inflammatory bowel disease (IBD) (for example, and not limited to, Crohn's disease (CD), clonal disease, ulcerative colitis, collagen colitis, lymphocytic colitis, ischemic colitis, empty colitis, Behcet's syndrome, infectious colitis, indeterminate colitis, interstitial Cystitis), lupus (for example, and not limited to, systemic lupus erythematosus, discoid lupus, subacute cutaneous lupus erythematosus, cutaneous lupus erythematosus (such as chilblain lupus erythematosus), drug-induced lupus, neonatal lupus, lupus nephritis), mixed connective tissue disease, morphea, multiple sclerosis (MS), severe muscle Force disorder, narcolepsy, neuromuscular angina, pemphigus vulgaris, pernicious anemia, psoriasis, psoriatic arthritis, polymyositis, primary biliary cirrhosis, relapsing polychondritis, schizophrenia, scleroderma, Sjogren's syndrome, systemic stiffness syndrome, temporal arteritis (also known as giant cell arteritis), vasculitis, vitiligo, Wegener's granulomatosis, transplant rejection-associated immune reaction(s) (for example, and not limited to, renal transplant rejection, lung transplant rejection, liver transplant rejection), psoriasis, Wiskott-Aldrich syndrome, autoimmune lymphoproliferative syndrome, myasthenia gravis, inflammatory chronic rhinosinusitis, colitis, celiac disease, Barrett's esophagus, inflammatory gastritis, autoimmune nephritis, autoimmune hepatitis, autoimmune carditis, autoimmune encephalitis, autoimmune mediated hematological disease, asthma, atopic dermatitis, atopy, allergy, allergic rhinitis, scleroderma, bronchitis, pericarditis, the inflammatory disease is, Alzheimer's disease, Parkinson's disease, amyotrophic lateral sclerosis, inflammatory lung disease, inflammatory skin disease, atherosclerosis, myocardial infarction, stroke, gram-positive shock, gram-negative shock, sepsis, septic shock, hemorrhagic shock, anaphylactic shock, systemic inflammatory response syndrome. Additionally or alternatively, the subject designated, by the method described herein in the section entitled “METHODS FOR ASSESSING A LIKELIHOOD OF A RESPONSE TO THERAPEUTIC AGENT(S),” as being likely to respond to the activatable therapeutic agent (such as one described herein) can be one with an expression profile of biomarker(s) such that, upon administering an activatable therapeutic agent (such as one described herein) to the subject, the activatable therapeutic agent is more likely than not to be cleaved at or near the target tissue(s) or cell(s) (such as described herein in the “Target Tissues or Cells” section), e.g., by mammalian protease(s), thereby activating the therapeutic agent.

Provided herein, in some embodiments, is use of a therapeutic agent (such as one described herein) or a pharmaceutical composition (such as one described herein) in the preparation of a medicament for the treatment of a disease or condition in a subject. The subject can be selected from mouse, rat, monkey, and human. The subject can be a human. The subject can be determined to have a likelihood of a response to the therapeutic agent or the pharmaceutical composition. The likelihood of the response can be 50% or higher. The likelihood of the response can be determined by a method as described herein (such as one described hereinabove in the METHODS FOR ASSESSING A LIKELIHOOD OF A RESPONSE TO THERAPEUTIC AGENT(S) section). In some embodiments, the disease or condition can be a cancer or an inflammatory or autoimmune disease. In some embodiments, the disease or condition can be a cancer. The cancer can be selected from the group consisting of carcinoma, Hodgkin's lymphoma, and non-Hodgkin's lymphoma, diffuse large B cell lymphoma, follicular lymphoma, mantle cell lymphoma, blastoma, breast cancer, ER/PR+ breast cancer, Her2+ breast cancer, triple-negative breast cancer, colon cancer, colon cancer with malignant ascites, mucinous tumors, prostate cancer, head and neck cancer, skin cancer, melanoma, genito-urinary tract cancer, ovarian cancer, ovarian cancer with malignant ascites, peritoneal carcinomatosis, uterine serous carcinoma, endometrial cancer, cervix cancer, colorectal, uterine cancer, mesothelioma in the peritoneum, kidney cancer, Wilm's tumor, lung cancer, small-cell lung cancer, non-small cell lung cancer, gastric cancer, stomach cancer, small intestine cancer, liver cancer, hepatocarcinoma, hepatoblastoma, liposarcoma, pancreatic cancer, gall bladder cancer, cancers of the bile duct, esophageal cancer, salivary gland carcinoma, thyroid cancer, epithelial cancer, arrhenoblastoma, adenocarcinoma, sarcoma, and B-cell derived chronic lymphatic leukemia. In some embodiments, the disease or condition can be an inflammatory or autoimmune disease. The inflammatory or autoimmune disease can be selected from the group consisting of ankylosing spondylitis (AS), arthritis (for example, and not limited to, rheumatoid arthritis (RA), juvenile idiopathic arthritis (JIA), osteoarthritis (OA), psoriatic arthritis (PsA), gout, chronic arthritis), chagas disease, chronic obstructive pulmonary disease (COPD), dermatomyositis, type 1 diabetes, endometriosis, Goodpasture syndrome, Graves' disease, Guillain-Barre syndrome (GB S), Hashimoto's disease, suppurative scab, Kawasaki disease, IgA nephropathy, idiopathic thrombocytopenic purpura, inflammatory bowel disease (IBD) (for example, and not limited to, Crohn's disease (CD), clonal disease, ulcerative colitis, collagen colitis, lymphocytic colitis, ischemic colitis, empty colitis, Behcet's syndrome, infectious colitis, indeterminate colitis, interstitial Cystitis), lupus (for example, and not limited to, systemic lupus erythematosus, discoid lupus, subacute cutaneous lupus erythematosus, cutaneous lupus erythematosus (such as chilblain lupus erythematosus), drug-induced lupus, neonatal lupus, lupus nephritis), mixed connective tissue disease, morphea, multiple sclerosis (MS), severe muscle Force disorder, narcolepsy, neuromuscular angina, pemphigus vulgaris, pernicious anemia, psoriasis, psoriatic arthritis, polymyositis, primary biliary cirrhosis, relapsing polychondritis, schizophrenia, scleroderma, Sjogren's syndrome, systemic stiffness syndrome, temporal arteritis (also known as giant cell arteritis), vasculitis, vitiligo, Wegener's granulomatosis, transplant rejection-associated immune reaction(s) (for example, and not limited to, renal transplant rejection, lung transplant rejection, liver transplant rejection), psoriasis, Wiskott-Aldrich syndrome, autoimmune lymphoproliferative syndrome, myasthenia gravis, inflammatory chronic rhinosinusitis, colitis, celiac disease, Barrett's esophagus, inflammatory gastritis, autoimmune nephritis, autoimmune hepatitis, autoimmune carditis, autoimmune encephalitis, autoimmune mediated hematological disease, asthma, atopic dermatitis, atopy, allergy, allergic rhinitis, scleroderma, bronchitis, pericarditis, the inflammatory disease is, Alzheimer's disease, Parkinson's disease, amyotrophic lateral sclerosis, inflammatory lung disease, inflammatory skin disease, atherosclerosis, myocardial infarction, stroke, gram-positive shock, gram-negative shock, sepsis, septic shock, hemorrhagic shock, anaphylactic shock, systemic inflammatory response syndrome. Additionally or alternatively, the subject designated, by the method described herein in the section entitled “METHODS FOR ASSESSING A LIKELIHOOD OF A RESPONSE TO THERAPEUTIC AGENT(S),” as being likely to respond to the activatable therapeutic agent (such as one described herein) can be one with an expression profile of biomarker(s) such that, upon administering an activatable therapeutic agent (such as one described herein) to the subject, the activatable therapeutic agent is more likely than not to be cleaved at or near the target tissue(s) or cell(s) (such as described herein in the “Target Tissues or Cells” section), e.g., by mammalian protease(s), thereby activating the therapeutic agent.

### EXAMPLES

### Example 1. Recombinant Production of an XTENylated Fusion Polypeptide Containing an Exemplary Peptide Substrate

This example illustrates recombinant construction, production, and purification of an XTENylated fusion polypeptide containing an exemplary peptide substrate using the methods disclosed herein.

EXPRESSION: Constructs encoding an XTENylated fusion polypeptide comprising an amino acid sequence of SEQ ID NO: 20 or 22, containing two elastin-based peptide substrates, both of the sequence GPGG-VAAA (SEQ ID NO: 1283) (shown in #527 of Column II of Table A), are expressed in a proprietary E. coli AmE098 strain and partitioned into the periplasm via an N-terminal secretory leader sequence (MKKNIAFLLASMFVFSIATNAYA-) (SEQ ID NO: 3129), which is cleaved during translocation. Fermentation cultures are grown with animal-free complex medium at 37° C.; and the temperature is shifted to 26° C. prior to phosphate depletion. During harvest, fermentation whole broth is centrifuged to pellet the cells. At harvest, the total volume and the wet cell weight (WCW; ratio of pellet to supernatant) is recorded, and the pelleted cells are collected and frozen at −80° C.

RECOVERY: The frozen cell pellet is resuspended in Lysis Buffer (17.7 mM citric acid, 22.3 mM Na₂HPO₄, 75 mM NaCl, 2 mM EDTA, pH 4.0) targeting 30% wet cell weight. The resuspension is allowed to equilibrate at pH 4 then homogenized via two passes at 800±50 bar while output temperature is monitored and maintained at 15±5° C. The pH of the homogenate is confirmed to be within the specified range (pH 4.0±0.2).

CLARIFICATION: To reduce endotoxin and host cell impurities, the homogenate is allowed to undergo low-temperature (10±5° C.), acidic (pH 4.0±0.2) flocculation overnight (15-20 hours). To remove the insoluble fraction, the flocculated homogenate is centrifuged for 40 minutes at 16,900 RCF at 2-8° C., and the supernatant is retained. The supernatant is diluted approximately 3-fold with Milli-Q water (MQ), then adjusted to 7±1 mS/cm with 5 M NaCl. To remove nucleic acid, lipids, and endotoxin and to act as a filter aid, the supernatant is adjusted to 0.1% (m/m) diatomaceous earth. To keep the filter aid suspended, the supernatant is mixed via impeller and allowed to equilibrate for 30 minutes. A filter train, consisting of a depth filter followed by a 0.22 μm filter, is assembled then flushed with MQ. The supernatant is pumped through the filter train while modulating flow to maintain a pressure drop of 25±5 psig. To adjust the composite buffer system (based on the ratio of citric acid and Na₂HPO₄) to the desired range for capture chromatography, the filtrate is adjusted with 500 mM Na₂HPO₄ such that the final ratio of Na₂HPO₄ to citric acid is 9.33:1, and the pH of the buffered filtrate is confirmed to be within the specified range (pH 7.0±0.2).

Purification

AEX Capture: To separate dimer, aggregate, and large truncates from monomeric product, and to remove endotoxin and nucleic acids, anion exchange (AEX) chromatography is utilized to capture the electronegative C-terminal XTEN domain. The AEX1 stationary phase (GE Q Sepharose FF), AEX1 mobile phase A (12.2 mM Na₂HPO₄, 7.8 mM Na₂HPO₄, 40 mM NaCl), and AEX1 mobile phase B (12.2 mM Na₂HPO₄, 7.8 mM Na₂HPO₄, 500 mM NaCl) are used herein. The column is equilibrated with AEX1 mobile phase A. Based on the total protein concentration measured by bicinchoninic acid (BCA) assay, the filtrate is loaded onto the column targeting 28±4 g/L-resin, chased with AEX1 mobile phase A, then washed with a step to 30% B. Bound material is eluted with a gradient from 30% B to 60% B over 20 CV. Fractions are collected in 1 CV aliquots while A220≥100 mAU above (local) baseline. Elution fractions are analyzed and pooled on the basis of SDS-PAGE and SE-HPLC.

IMAC Intermediate Purification: To ensure C-terminal integrity, immobilized metal affinity chromatography (IMAC) is used to capture the C-terminal polyhistidine tag (His(6)). The IMAC stationary phase (GE IMAC Sepharose FF), IMAC mobile phase A (18.3 mM Na₂HPO₄, 1.7 mM Na₂HPO₄, 500 mM NaCl, 1 mM imidazole), and IMAC mobile phase B (18.3 mM Na₂HPO₄, 1.7 mM Na₂HPO₄, 500 mM NaCl, 500 mM imidazole) are used herein. The column is charged with zinc solution and equilibrated with IMAC mobile phase A. The AEX1 Pool is adjusted to pH 7.8±0.1, 50±5 mS/cm (with 5 M NaCl), and 1 mM imidazole, loaded onto the IMAC column targeting 2 g/L-resin, and chased with IMAC mobile phase A until absorbance at 280 nm (A280) returned to (local) baseline. Bound material is eluted with a step to 25% IMAC mobile phase B. The IMAC Elution collection is initiated when A280≥10 mAU above (local) baseline, directed into a container pre-spiked with EDTA sufficient to bring 2 CV to 2 mM EDTA, and terminated once 2 CV were collected. The elution is analyzed by SDS-PAGE.

Protein-L Intermediate Purification: To ensure N-terminal integrity, Protein-L is used to capture kappa domains present close to the N-terminus of the fusion polypeptide (specifically the aEpCAM scFv). Protein-L stationary phase (GE Capto L), Protein-L mobile phase A (16.0 mM citric acid, 20.0 mM Na₂HPO₄, pH 4.0±0.1), Protein-L mobile phase B (29.0 mM citric acid, 7.0 mM Na₂HPO₄, pH 2.60±0.02), and Protein-L mobile phase C (3.5 mM citric acid, 32.5 mM Na₂HPO₄, 250 mM NaCl, pH 7.0±0.1) are used herein. The column is equilibrated with Protein-L mobile phase C. The IMAC Elution is adjusted to pH 7.0±0.1 and 30±3 mS/cm (with 5 M NaCl and MQ) and loaded onto the Protein-L column targeting 2 g/L-resin then chased with Protein-L mobile phase C until absorbance at 280 nm (A280) returns to (local) baseline. The column is washed with Protein-L mobile phase A, and Protein-L mobile phases A and B are used to effect low-pH elution. Bound material is eluted at approximately pH 3.0 and collected into a container pre-spiked with one part 0.5 M Na₂HPO₄ for every 10 parts collected volume. Fractions are analyzed by SDS-PAGE.

HIC Polishing: To separate N-terminal variants (4 residues at the absolute N-terminus are not essential for Protein-L binding) and overall conformation variants, hydrophobic interaction chromatography (HIC) is used. HIC stationary phase (GE Capto Phenyl ImpRes), HIC mobile phase A (20 mM histidine, 0.02% (w/v) polysorbate 80, pH 6.5±0.1) and HIC mobile phase B (1 M ammonium sulfate, 20 mM histidine, 0.02% (w/v) polysorbate 80, pH 6.5±0.1) are used herein. The column is equilibrated with HIC mobile phase B. The adjusted Protein-L Elution is loaded onto the HIC column targeting 2 g/L-resin and chased with HIC mobile phase B until absorbance at 280 nm (A280) returned to (local) baseline. The column is washed with 50% B. Bound material is eluted with a gradient from 50% B to 0% B over 75 CV. Fractions are collected in 1 CV aliquots while A280≥3 mAU above (local) baseline. Elution fractions are analyzed and pooled on the basis of SE-HPLC and HI-HPLC.

FORMULATION: To exchange the product into formulation buffer and to bring the product to the target concentration (0.5 g/L), anion exchange is again used to capture the C-terminal XTEN. AEX2 stationary phase (GE Q Sepharose FF), AEX2 mobile phase A (20 mM histidine, 40 mM NaCl, 0.02% (w/v) polysorbate 80, pH 6.5±0.2), AEX2 mobile phase B (20 mM histidine, 1 M NaCl, 0.02% (w/v) polysorbate 80, pH 6.5±0.2), and AEX2 mobile phase C (12.2 mM Na₂HPO₄, 7.8 mM NaH₂PO₄, 40 mM NaCl, 0.02% (w/v) polysorbate 80, pH 7.0±0.2) are used herein. The column is equilibrated with AEX2 mobile phase C. The HIC Pool is adjusted to pH 7.0±0.1 and 7±1 mS/cm (with MQ) and loaded onto the AEX2 column targeting 2 g/L-resin then chased with AEX2 mobile phase C until A280 returned to (local) baseline. The column is washed with AEX2 mobile phase A (20 mM histidine, 40 mM NaCl, 0.02% (w/v) polysorbate 80, pH 6.5±0.2). AEX2 mobile phases A and B are used to generate an [NaCl] step and effect elution. Bound material is eluted with a step to 38% AEX2 mobile phase B. The AEX2 Elution collection is initiated when A280≥5 mAU above (local) baseline and terminated once 2 CV were collected. The AEX2 Elution is 0.22 μm filtered within a BSC, aliquoted, labeled, and stored at −80° C. as Bulk Drug Substance (BDS). The bulk drug substance (BDS) is confirmed by various analytical methods to meet all lot release criteria. Overall quality is analyzed by SDS-PAGE, the ratio of monomer to dimer and aggregate is analyzed by SE-HPLC, and N-terminal quality and product homogeneity are analyzed by HI-HPLC.

### Example 2. Preparation of Plasma Samples

This example illustrates preparation of plasma samples from patients suffering from, or is suspected of suffering from, a disease or condition known to be associated with an elevated level of elastin at or near a diseased site.

Blood is collected from a patient of choice into an EDTA plasma tube and centrifuged for 10 minutes at 4° C. and 3,500 g. Plasma is then aliquoted and flash-frozen on dry ice within 30 minutes of collection. 250 μL aliquots of plasma are later thawed on ice and precipitated with 1 mL of water containing 80% acetonitrile and 1 nanogram (ng) of bovine insulin as an internal standard. The solid phase extraction eluant is transferred and evaporated to dryness, then diluted with 75 μL of water with 0.1% formic acid, thereby obtaining a sample of plasma peptides.

Possible variations in sample preparation, including those for a nano LC/MS, may be found in Kay et al. 2018 (Rapid Communications in Mass Spectrometry 32 (16), 1414-1424, 2018.

### Example 3. Liquid Chromatography-Mass Spectrometry (LC-MS)

This example illustrates liquid chromatography-mass spectrometry (LC-MS) methods used to determine the presence and/or amount of biomarker peptides in plasma samples from subjects using the methods disclosed herein.

50 μL of the plasma peptides as obtained according to Example 2 is injected into a liquid chromatography-mass spectrometry (LC-MS) system with a high flow configuration. Two buffers, buffer A (0.1% formic acid in water) and buffer B (0.1% formic acid in 80:20 acetonitrile/water), for liquid chromatography (LC) separations are prepared. 50 μL of sample extract is injected into a HSS T3 column (2.1×50 mm) at 15% buffer A and 85% buffer B with a flow rate of 300 μL/min, then separated to 40% buffer B using a 6.5 minute gradient. The column is then washed at 90% buffer B for 1.5 minutes and returned to initial conditions after 8 minutes. A scan from 600 mass per charge (m/z) to 1,600 m/z is conducted for information-dependent acquisition using a resolution of 75,000, a maximum fill time of 200 ms, and an automatic gain control of 3×10⁶.

Peptides are identified using Peaks 8.0 software searched against the human Swissprot database. The search configuration includes precursor and product ion tolerances of 10 ppm and 0.05 Da (respectively), the no-digest setting, a false discovery rate threshold of 1%, and allowance of modifications such as C-terminal amidation.

### Example 4. Matrix-Assisted Laser Desorption/Ionization-Time of Flight (MALDI-TOF) Mass Spectrometry

This example illustrates matrix-assisted laser desorption/ionization-time of flight (MALDI-TOF) mass spectrometry methods used to determine the presence and/or amount of biomarker peptides in plasma samples from subjects using the methods disclosed herein.

As an alternative to Example 3, plasma peptides obtained according to Example 2 is isolated by loading plasma samples, mixed in a 3:1 ratio with a solution of 20% acetonitrile and 1% trifluoroacetic acid, onto nanoporous silica chips for analysis by a matrix-assisted laser desorption/ionization-time of flight (MALDI-TOF) mass spectrometer, as described in details in Bedin et al. 2015 (J Cell Physiol., 231(4):915-25). The plasma peptides are identified using Mascot and MS-Tag search engines with preprocessing steps performed by flexAnalysis and Snap™ softwares. The presence or/and amount of the plasma peptides having (i) a sequence of GVAPGIGPGG (shown in #527 of column IV of Table A), or (ii) a sequence of VAAAAKSAAK (SEQ ID NO. 3116; shown in #527 of column VI of Table A) (or a fragment thereof) is determined.

### Example 5. Enzyme-Linked Immunosorbent Assay (ELISA)

This example illustrates immunoassay methods used to determine the presence and/or amount of biomarker peptides in plasma samples from subjects using the methods disclosed herein.

Capture antibodies specific to one or more biomarker(s) of (i) a sequence of GVAPGIGPGG (SEQ ID NO: ) (shown in #527 of column IV of Table A), (ii) a sequence of VAAAAKSAAK (SEQ ID NO: ) (shown in #527 of column VI of Table A), and (iii) a sequence of GPGGVAAA (SEQ ID NO: ) (shown in #527 of column II of Table A) (or a fragment thereof) are obtained.

The plasma sample obtained according to Example 2 is diluted and the plasma concentrations of the biomarker peptide(s) are measured using a competitive ELISA. Primary antibody (unlabeled) is incubated with sample antigen. Antibody-antigen complexes are then added to 96-well plates which are pre-coated with the same antigen. Unbound antibody is removed by washing the plate. (The more antigen in the sample, the less antibody will be able to bind to the antigen in the well, hence “competition.”) The secondary antibody that is specific to the primary antibody and conjugated with an enzyme is added. A substrate is added, and remaining enzymes elicit a chromogenic or fluorescent signal.

### Example 6. Patient Designations

This example illustrates designating patients as being likely to respond to activatable therapeutic agents using the methods disclosed herein.

The presence or/and amount of biomarker peptide(s) as determined according to one of Examples 3-5 is analyzed manually or with semi-automated/automated procedures/instruments. If the biomarker peptide(s) is/determined to be present in the plasma sample from the patient, or if the amount of biomarker peptide(s) of the patient is determined to exceed a pre-determined threshold, the patient is designated as having a likeliness of more than 50% to respond to the therapeutic agent constructed and produced according to Example 1 which comprises the elastin-based peptide substrate (shown in #527 of Column II of Table A) in its release segment.

### Example 7. Assessment of Protease Cleavage of Release Segments Having Collagen I Derived Amino Acid Sequences

This invention provides non-natural, activatable therapeutic agents (e.g. XPATs) wherein a biologically active moiety (BM) is preferentially released at a target site associated with expression of a mammalian protease that cleaves a scissile bond in a release segment linked directly or indirectly to the BM. Successful therapeutic use of these agents in an individual depends on whether the agent comprises a release segment linked directly or indirectly to the BM that is cleaved by a mammalian protease expressed at a target site in that individual. An assessment of whether an individual having a target site to be targeted for delivery and release of the BM expresses a mammalian protease that cleaves a release segment can be valuable in identifying and matching therapeutically effective agents for a particular individual. Achieving such a beneficial assessment is dependent on determining the relative efficiency of cleavage of release segment sequences by mammalian proteases known to be expressed at therapeutic target sites, such as tumors and inflammatory sites.

Set forth in this example are the results of experiments that demonstrated unmasking rates of ECP-based release sites. The substrates 818-P1, C1MA, and C1 MB were digested by proteases and cleavage rates measured.

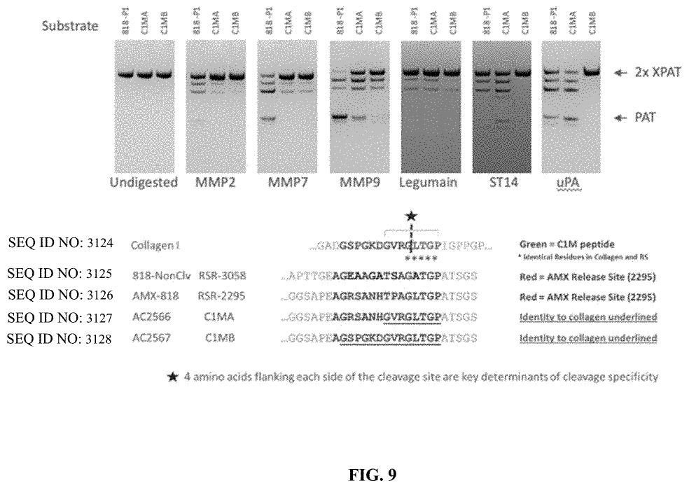

Protease digestion was performed under varying conditions and were based on comparison of 818-C1MA and 818-C1 MB to 818-P1 digestion. Substrate (1 μM) was digested at 37° C. with MMPs for two hours, Legumain and ST14 for four hours, or Urokinase-type Plasminogen Activator (uPA) for 6 hours as shown in Table 8. Digestion buffers varied in composition and enzyme concentration, MMP (5 nM), Legumain, ST14 (50 nM) and uPA (100 nM). Cleavage of 818-P1, C1MA and C1 MB at lysine/leucine residues similar to collagen (a known component of the extracellular matrix, ECM) are demonstrated in FIG. 9.

Results demonstrated that MMP 2, 7, and 9 unmasked 818-P1 faster than 818-C1MA and 818-C1 MB (MMP2: 818-P1>818-C1MA>818-C1 MB; MMP1: 818-P1>818-C1MA=818-C1 MB; MMP9: 818-P1>818-C1 MB>818-C1MA). Legumain and ST 14 required a higher concentration and longer time for unmasking. Legumain demonstrated minimal unmasking differences whereas ST14 unmaking was characterized by 818-C1MA>818-P1>818-C1 MB. Unmasking activity attributable to uPA required higher concentrations of proteases and longer digestion times.

Proteases expressed during cancer growth and metastasis remodel the ECM and can lead to elevated plasma levels of ECM protein cleavage products that are elevated in the plasma of patients with a wide variety of tumors. The current example demonstrates that a cleavage product resulting from MMP cleavage of an ECM protein is highly similar to the MMP cleavage site in protease-cleavable linkers in XPATs. These results demonstrated that the protease cleavable linker employed in the XPATs of this invention are more efficiently cleaved than the ECM by purified MMPs and that the presence of ECM peptides in cancer patients can serve as an indicator that the patients' tumors are expressing MMPs that can cleave the protease-cleavable linker in an XPAT, thereby predicting whether a given patient or tumor will be able to cleave the XPAT and hence result in treatment of the tumor. This allows for a personalized approach to determine whether an XPAT will be cleaved in a given tumor type by determining whether the subject that has said tumor type has elevated plasma levels of certain cleavage product(s) derived from the extracellular matrix.

TABLE 8
Protease Sources and Partial Digest Conditions

| | Protease | | |
|---|---|---|---|
| | Conc | Time | |
| Protease | (nM) | (hr) | Digest Buffer |
| MMP2 | 5 | 2 | 20 mM Histidine, 154 mM NaCl, |
| | | | 0.005% PS-80, 10 mM CaCl2, pH 6.5 |
| MMP7 | 5 | 2 | 20 mM Histidine, 154 mM NaCl, |
| | | | 0.005% PS-80, 10 mM CaCl2, pH 6.5 |
| MMP9 | 5 | 2 | 20 mM Histidine, 154 mM NaCl, |
| | | | 0.005% PS-80, 10 mM CaCl2, pH 6.5 |
| Legumain | 50 | 4 | 50 mM MES, 250 mM NaCl, pH 5.0 |
| ST14/ | 50 | 4 | 20 mM Histidine, 154 mM NaCl, |
| Matriptase | | | 0.005% PS-80, 10 mM CaCl2, pH 6.5 |
| uPA | 100 | 6 | 50 mM Tris-HCl, pH 8.0 |
| Trypsin | N/A (~20 μL | 0.5-2 | PBS |
| (immo- | slurry/ | | |
| bilized) | 100 μL) | | |

TABLE 9
Protease Cleavage Release Segment Sequences

| Name | SEQ ID NO | Sequence |
|---|---|---|
| Collagen I | 3124 | GADGSPGKDGVRGLTGPIGPPGP |
| 818-NonClv | 3225 | APTTGEAGEAAGATSAGATGPATSGS |
| AMX-818 | 3126 | GGSAPEAGRSANHTPAGLTGPATSGS |
| AC2566 | 3127 | GGSAPEAGRSANHGVRGLTGPATSGS |
| AC2567 | 3128 | GGSAPEAGSPGKDGVRGLTGPATSGS |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for assessing a likelihood of a subject being responsive to a therapeutic agent that is activatable by a mammalian protease expressed in said subject having a disease or disorder, the method comprising:
a. determining, in a biological sample from said subject affected by the disease or disorder, a presence or an amount of a proteolytic peptide product produced by action of said mammalian protease, wherein said peptide
i. comprises at least five or six consecutive amino acid residues shown in a sequence set forth in Column V of Table A; or
ii. comprises at least five or six consecutive amino acids shown in a sequence set forth in Column IV of Table A; or
iii. comprises at least five or six consecutive amino acids shown in a sequence set forth in Column VI of Table A; and
b. designating said subject as being likely to respond to said therapeutic agent when said peptide of (i), (ii) or (iii) is present and/or if its amount exceeds a threshold value.

2. The method of claim 1, wherein:
a. said therapeutic agent comprises a peptide substrate having an amino acid sequence that is susceptible to cleavage by said mammalian protease at a scissile bond;
b. said polypeptide of (i), (ii), or (iii) comprises a portion containing at least four, at least five, at least six, at least seven, at least eight, at least nine, or at least ten consecutive amino acid residues of said sequence of the peptide substrate that is either N-terminal or C-terminal side of said scissile bond;
c. said sequence of the peptide substrate is susceptible to cleavage by said mammalian protease at a scissile bond, and wherein said polypeptide of (i), (ii), or (iii) is a cleavage product of a reporter polypeptide comprising a substrate sequence that is susceptible to cleavage by the same mammalian protease at a scissile bond and where said reporter polypeptide comprises a sequence set forth in Column II or III of Table A; and/or
d. said sequence of the peptide substrate is susceptible to cleavage by said mammalian protease at a scissile bond, and wherein said polypeptide of (i), (ii), or (iii) is a cleavage product of a human protein that comprises a portion containing at least five or six consecutive amino acid residues of said peptide substrate sequence that includes the scissile bond;
e. said polypeptide of (i) comprises at least seven, at least eight, at least nine, or at least ten consecutive amino acid residues shown in a sequence set forth in Column V of Table A;
f. said polypeptide of (ii) comprises at least seven, at least eight, at least nine, or at least ten consecutive amino acids shown in a sequence set forth in Column IV of Table A;
g. said polypeptide of (iii) comprises at least seven, at least eight, at least nine, or at least ten consecutive amino acids shown in a sequence set forth in Column VI of Table A; and/or
h. step (a) comprises determining the presence or the amount of any two of (i)-(iii).

3-9. (canceled)

10. The method of claim 1, wherein:
a. said threshold is zero or nominal;
b. said biological sample comprises a serum or plasma sample;
c. said mammalian protease is a serine protease, a cysteine protease, an aspartate protease, a threonine protease, or a metalloproteinase; optionally wherein:
i. said mammalian protease is selected from the group consisting of disintegrin and metalloproteinase domain-containing protein 10 (ADAM10), disintegrin and metalloproteinase domain-containing protein 12 (ADAM12), disintegrin and metalloproteinase domain-containing protein 15 (ADAM15), disintegrin and metalloproteinase domain-containing protein 17 (ADAM17), disintegrin and metalloproteinase domain-containing protein 9 (ADAM9), disintegrin and metalloproteinase with thrombospondin motifs 5 (ADAMTS5), Cathepsin B, Cathepsin D, Cathepsin E, Cathepsin K, cathepsin L, cathepsin S, Fibroblast activation protein alpha, Hepsin, kallikrein-2, kallikrein-4, kallikrein-3, Prostate-specific antigen (PSA), kallikrein-13, Legumain, matrix metallopeptidase 1 (MMP-1), matrix metallopeptidase 10 (MMP-10), matrix metallopeptidase 11 (MMP-11), matrix metallopeptidase 12 (MMP-12), matrix metallopeptidase 13 (MMP-13), matrix metallopeptidase 14 (MMP-14), matrix metallopeptidase 16 (MMP-16), matrix metallopeptidase 2 (MMP-2), matrix metallopeptidase 3 (MMP-3), matrix metallopeptidase 7 (MMP-7), matrix metallopeptidase 8 (MMP-8), matrix metallopeptidase 9 (MMP-9), matrix metallopeptidase 4 (MMP-4), matrix metallopeptidase 5 (MMP-5), matrix metallopeptidase 6 (MMP-6), matrix metallopeptidase 15 (MMP-15), neutrophil elastase, protease activated receptor 2 (PAR2), plasmin, prostasin, PSMA-FOLH1, membrane type serine protease 1 (MT-SP1), matriptase, and u-plasminogen; or
ii. said mammalian protease is selected from the group consisting of matrix metallopeptidase 1 (MMP1), matrix metallopeptidase 2 (MMP2), matrix metallopeptidase 7 (MMP7), matrix metallopeptidase 9 (MMP9), matrix metallopeptidase 11 (MMP11), matrix metallopeptidase 14 (MMP14), urokinase-type plasminogen activator (uPA), legumain, and matriptase.

11-14. (canceled)

15. The method of claim 1, wherein:
a. said mammalian protease is preferentially expressed or activated in a target tissue or cell;
b. said target tissue or cell is a tumor;
c. said target tissue or cell produces or is co-localized with said mammalian protease;
d. said target tissue or cell contains therein or thereon, or is associated within proximity thereto, a reporter polypeptide; and/or
e. said target tissue or cell is characterized by an increased amount or activity of said mammalian protease in proximity to said target tissue or cell as compared to a non-target tissue or cell in said subject.

16-18. (canceled)

19. The method of claim 2, wherein said reporter polypeptide:
a. is a polypeptide selected from the group consisting of coagulation factor, complement component, tubulin, immunoglobulin, apolipoprotein, serum amyloid, insulin, growth factor, fibrinogen, PDZ domain protein, LIM domain protein, c-reactive protein, serum albumin, versican, collagen, elastin, keratin, kininogen-1, alpha-2-antiplasmin, clusterin, biglycan, alpha-1-antitrypsin, transthyretin, alpha-1-antichymotrypsin, glucagon, hepcidin, thymosin beta-4, haptoglobin, hemoglobin subunit alpha, caveolae-associated protein 2, alpha-2-HS-glycoprotein, chromogranin-A, vitronectin, hemopexin, epididymis secretory sperm binding protein, secretogranin-2, angiotensinogen, transgelin-2, pancreatic prohormone, neurosecretory protein VGF, ceruloplasmin, PDZ and LIM domain protein 1, multimerin-1, inter-alpha-trypsin inhibitor heavy chain H2, N-acetylmuramoyl-L-alanine amidase, histone H1.4, adhesion G-protein coupled receptor G6, mannan-binding lectin serine protease 2, prothrombin, deleted in malignant brain tumors 1 protein, desmoglein-3, calsyntenin-1, alpha-2-macroglobulin, myosin-9, sodium/potassium-transporting ATPase subunit gamma, oncoprotein-induced transcript 3 protein, serglycin, histidine-rich glycoprotein, inter-alpha-trypsin inhibitor heavy chain H5, integrin alpha-IIb, membrane-associated progesterone receptor component 1, histone H1.2, rho GDP-dissociation inhibitor 2, zinc-alpha-2-glycoprotein, talin-1, secretogranin-1, neutrophil defensin 3, cytochrome P450 2E1, gastric inhibitory polypeptide, transcription initiation factor TFIID subunit 1, integral membrane protein 2B, pigment epithelium-derived factor, voltage-dependent N-type calcium channel subunit alpha-1B, ras GTPase-activating protein nGAP, type I cytoskeletal 17, sulfhydryl oxidase 1, homeobox protein Hox-B2, transcription factor SOX-10, E3 ubiquitin-protein ligase SIAH2, decorin, secreted protein acidic and rich in cysteine (SPARC), laminin gamma 1 chain, vimentin, and nidogen-1 (NID1);
b. is a polypeptide selected from the group consisting of versican, type II collagen alpha-1 chain, kininogen-1, complement C4-A, complement C4-B, complement C3, alpha-2-antiplasmin, clusterin, biglycan, elastin, fibrinogen alpha chain, alpha-1-antitrypsin, fibrinogen beta chain, type III collagen alpha-1 chain, serum amyloid A-1 protein, transthyretin, apolipoprotein A-I, apolipoprotein A-I Isoform 1, alpha-1-antichymotrypsin, glucagon, hepcidin, serum amyloid A-2 protein, thymosin beta-4, haptoglobin, hemoglobin subunit alpha, caveolae-associated protein 2, alpha-2-HS-glycoprotein, chromogranin-A, vitronectin, hemopexin, epididymis secretory sperm binding protein, zyxin, apolipoprotein secretogranin-2, angiotensinogen, c-reactive protein, serum albumin, transgelin-2, pancreatic prohormone, neurosecretory protein VGF, ceruloplasmin, PDZ and LEVI domain protein 1, tubulin alpha-4A chain, multimerin-1, inter-alpha-trypsin inhibitor heavy chain H2, apolipoprotein C-I, fibrinogen gamma chain, N-acetylmuramoyl-L-alanine amidase, immunoglobulin lambda variable 3-21, histone H1.4, adhesion G-protein coupled receptor G6, immunoglobulin lambda variable 3-25, immunoglobulin lambda variable 1-51, immunoglobulin lambda variable 1-36, mannan-binding lectin serine protease 2, immunoglobulin kappa variable 3-20, immunoglobulin kappa variable 2-30, insulin-like growth factor II, apolipoprotein A-II, probable non-functional immunoglobulin kappa variable 2D-24, prothrombin, coagulation factor IX, apolipoprotein L1, deleted in malignant brain tumors 1 protein, desmoglein-3, calsyntenin-1, immunoglobulin lambda constant 3, complement C5, alpha-2-macroglobulin, myosin-9, sodium/potassium-transporting ATPase subunit gamma, immunoglobulin kappa variable 2-28, oncoprotein-induced transcript 3 protein, serglycin, coagulation factor XII, coagulation factor XIII A chain, insulin, histidine-rich glycoprotein, immunoglobulin kappa variable 3-11, immunoglobulin kappa variable 1-39, collagen alpha-1(I) chain, inter-alpha-trypsin inhibitor heavy chain H5, latent-transforming growth factor beta-binding protein 2, integrin alpha-IIb, membrane-associated progesterone receptor component 1, immunoglobulin lambda variable 6-57, immunoglobulin kappa variable 3-15, complement C1r subcomponent-like protein, histone H1.2, rho GDP-dissociation inhibitor 2, latent-transforming growth factor beta-binding protein 4, collagen alpha-1(XVIII) chain, immunoglobulin lambda variable 2-18, zinc-alpha-2-glycoprotein, talin-1, secretogranin-1, neutrophil defensin 3, cytochrome P450 2E1, gastric inhibitory polypeptide, immunoglobulin heavy variable 3-15, immunoglobulin lambda variable 2-11, transcription initiation factor TFIID subunit 1, collagen alpha-1(VII) chain, integral membrane protein 2B, pigment epithelium-derived factor, voltage-dependent N-type calcium channel subunit alpha-1B, immunoglobulin lambda variable 3-27, ras GTPase-activating protein nGAP, keratin, type I cytoskeletal 17, tubulin beta chain, sulfhydryl oxidase 1, immunoglobulin kappa variable 4-1, complement C1r subcomponent, homeobox protein Hox-B2, transcription factor SOX-10, E3 ubiquitin-protein ligase SIAH2, decorin, SPARC, type I collagen alpha-1 chain, type IV collagen alpha-1 chain, laminin gamma 1 chain, vimentin, type III collagen, type IV collagen alpha-3 chain, type VII collagen alpha-1 chain, type VI collagen alpha-1 chain, type V collagen alpha-1 chain, nidogen-1, and type VI collagen alpha-3 chain; and/or
c. comprises a sequence set forth in Columns II-VI of Table A.

20-22. (canceled)

23. The method of claim 1, wherein said subject is suffering from, or is suspected of suffering from, a disease or condition characterized by an increased expression or activity of said mammalian protease in proximity to a target tissue or cell as compared to a corresponding non-target tissue or cell in said subject; optionally wherein:
a. said disease or condition is a cancer or an inflammatory or autoimmune disease;
b. said disease or condition is selected from the group consisting of carcinoma, Hodgkin's lymphoma, and non-Hodgkin's lymphoma, diffuse large B cell lymphoma, follicular lymphoma, mantle cell lymphoma, blastoma, breast cancer, ER/PR+ breast cancer, Her2+ breast cancer, triple-negative breast cancer, colon cancer, colon cancer with malignant ascites, mucinous tumors, prostate cancer, head and neck cancer, skin cancer, melanoma, genito-urinary tract cancer, ovarian cancer, ovarian cancer with malignant ascites, peritoneal carcinomatosis, uterine serous carcinoma, endometrial cancer, cervix cancer, colorectal, uterine cancer, mesothelioma in the peritoneum, kidney cancer, Wilm's tumor, lung cancer, small-cell lung cancer, non-small cell lung cancer, gastric cancer, stomach cancer, small intestine cancer, liver cancer, hepatocarcinoma, hepatoblastoma, liposarcoma, pancreatic cancer, gall bladder cancer, cancers of the bile duct, esophageal cancer, salivary gland carcinoma, thyroid cancer, epithelial cancer, arrhenoblastoma, adenocarcinoma, sarcoma, and B-cell derived chronic lymphatic leukemia; or
c. wherein said disease or condition is selected from the group consisting of ankylosing spondylitis (AS), arthritis (for example, and not limited to, rheumatoid arthritis (RA), juvenile idiopathic arthritis (JIA), osteoarthritis (OA), psoriatic arthritis (PsA), gout, chronic arthritis), chagas disease, chronic obstructive pulmonary disease (COPD), dermatomyositis, type 1 diabetes, endometriosis, Goodpasture syndrome, Graves' disease, Guillain-Barre syndrome (GBS), Hashimoto's disease, suppurative scab, Kawasaki disease, IgA nephropathy, idiopathic thrombocytopenic purpura, inflammatory bowel disease (IBD) (for example, and not limited to, Crohn's disease (CD), clonal disease, ulcerative colitis, collagen colitis, lymphocytic colitis, ischemic colitis, empty colitis, Behcet's syndrome, infectious colitis, indeterminate colitis, interstitial Cystitis), lupus (for example, and not limited to, systemic lupus erythematosus, discoid lupus, subacute cutaneous lupus erythematosus, cutaneous lupus erythematosus (such as chilblain lupus erythematosus), drug-induced lupus, neonatal lupus, lupus nephritis), mixed connective tissue disease, morphea, multiple sclerosis (MS), severe muscle Force disorder, narcolepsy, neuromuscular angina, pemphigus vulgaris, pernicious anemia, psoriasis, psoriatic arthritis, polymyositis, primary biliary cirrhosis, relapsing polychondritis, schizophrenia, scleroderma, Sjogren's syndrome, systemic stiffness syndrome, temporal arteritis (also known as giant cell arteritis), vasculitis, vitiligo, Wegener's granulomatosis, transplant rejection-associated immune reaction(s) (for example, and not limited to, renal transplant rejection, lung transplant rejection, liver transplant rejection), psoriasis, Wiskott-Aldrich syndrome, autoimmune lymphoproliferative syndrome, myasthenia gravis, inflammatory chronic rhinosinusitis, colitis, celiac disease, Barrett's esophagus, inflammatory gastritis, autoimmune nephritis, autoimmune hepatitis, autoimmune carditis, autoimmune encephalitis, autoimmune mediated hematological disease, asthma, atopic dermatitis, atopy, allergy, allergic rhinitis, scleroderma, bronchitis, pericarditis, the inflammatory disease is, Alzheimer's disease, Parkinson's disease, amyotrophic lateral sclerosis, inflammatory lung disease, inflammatory skin disease, atherosclerosis, myocardial infarction, stroke, gram-positive shock, gram-negative shock, sepsis, septic shock, hemorrhagic shock, anaphylactic shock, systemic inflammatory response syndrome.

24-26. (canceled)

27. The method of claim 1, wherein said therapeutic agent:
a. is an anti-cancer agent;
b. is an activatable therapeutic agent;
c. further comprises a masking moiety (MM); optionally wherein:
i. said masking moiety (MM) is capable of being released from said therapeutic agent upon cleavage of said peptide substrate by said mammalian protease;
ii. said masking moiety (MM) interferes with an interaction of said therapeutic agent, in an uncleaved state, to a target tissue or cell;
iii. a bioactivity of said therapeutic agent is capable of being enhanced upon cleavage of said peptide substrate by said mammalian protease;
iv. said masking moiety is an extended recombinant polypeptide; optionally wherein said extended recombinant polypeptide is characterized in that (i) it comprises at least 100 amino acids; (ii) at least 90% of the amino acid residues of it are selected from glycine (G), alanine (A), serine (S), threonine (T), glutamate (E) and proline (P); and (iii) it comprises at least 4 different types of amino acids selected from G, A, S, T, E, and P.

28-34. (canceled)

35. The method of claim 1:
a. further comprises, assessing if a subject will be responsive to a therapeutic subsequent to (b), by contacting said therapeutic agent with said mammalian protease;
b. wherein (a) comprises detecting said polypeptide of (i), (ii) or (iii) in an immuno-assay; optionally wherein said immuno-assay utilizes an antibody that specifically binds to said polypeptide of (i), (ii) or (iii), or an epitope thereof;
c. wherein (a) comprises detecting said polypeptide of (i), (ii) or (iii) by using a mass spectrometer (MS); and/or
d. further comprises, subsequent to (b), administering to said subject an effective amount of said therapeutic agent based on the designation of step (b).

36-39. (canceled)

40. A method for treating a subject with an activatable therapeutic agent, the method comprising:
(a) identifying said subject as having a likelihood of a response to said activatable therapeutic agent based on identification of a peptide biomarker in a biological sample from said subject, which activatable therapeutic agent comprises a peptide substrate sequence susceptible to cleavage by a mammalian protease at a scissile bond; and
(b) administering said activatable therapeutic agent to said subject based on said identification of said subject in (a);
wherein said peptide biomarker comprises a portion identical to at least four consecutive amino acid residues of said peptide substrate sequence that is either N-terminal or C-terminal of said scissile bond.

41. The method of claim 40, wherein:
a. said peptide biomarker is derived from a reporter polypeptide, which reporter polypeptide comprises a sequence set forth in Columns II-VI of Table A;
b. said peptide biomarker has an amino acid sequence that is identical to a sequence of a reporter polypeptide, which reporter polypeptide comprises a sequence set forth in Columns II-VI of Table A;
c. said peptide substrate sequence contains from six to twenty-five or six to twenty amino acid residues; optionally wherein said peptide substrate sequence contains from seven to twelve amino acid residues;
d. said peptide substrate sequence comprises an amino acid sequence having at most three amino acid substitutions, at most two amino acid substitutions, or at most one amino acid substitution with respect to a sequence set forth in Column II or III of Table A, wherein none of said amino acid substitution is at a position corresponding to an amino acid residue immediately adjacent to a corresponding scissile bond as indicated in Table A; optionally wherein:
i. said peptide substrate sequence comprises an amino acid sequence set forth in Column II or III of Table A; or
ii. said peptide substrate sequence has an amino acid sequence identical to a fragment of a sequence set forth in Column II or III of Table A, wherein said fragment comprises at least four consecutive amino acid residues immediately adjacent to a corresponding scissile bond as indicated in Table A; optionally wherein said fragment contains at least five, at least six, at least seven, at least eight, at least nine, or at least ten amino acid residues
e. said biological sample comprises a serum or plasma sample;
f. said mammalian protease is a serine protease, a cysteine protease, an aspartate protease, a threonine protease, or a metalloproteinase; optionally wherein:
i. said mammalian protease is selected from the group consisting of disintegrin and metalloproteinase domain-containing protein 10 (ADAM10), disintegrin and metalloproteinase domain-containing protein 12 (ADAM12), disintegrin and metalloproteinase domain-containing protein 15 (ADAM15), disintegrin and metalloproteinase domain-containing protein 17 (ADAM17), disintegrin and metalloproteinase domain-containing protein 9 (ADAM9), disintegrin and metalloproteinase with thrombospondin motifs 5 (ADAMTS5), Cathepsin B, Cathepsin D, Cathepsin E, Cathepsin K, cathepsin L, cathepsin S, Fibroblast activation protein alpha, Hepsin, kallikrein-2, kallikrein-4, kallikrein-3, Prostate-specific antigen (PSA), kallikrein-13, Legumain, matrix metallopeptidase 1 (MMP-1), matrix metallopeptidase 10 (MMP-10), matrix metallopeptidase 11 (MMP-11), matrix metallopeptidase 12 (MMP-12), matrix metallopeptidase 13 (MMP-13), matrix metallopeptidase 14 (MMP-14), matrix metallopeptidase 16 (MMP-16), matrix metallopeptidase 2 (MMP-2), matrix metallopeptidase 3 (MMP-3), matrix metallopeptidase 7 (MMP-7), matrix metallopeptidase 8 (MMP-8), matrix metallopeptidase 9 (MMP-9), matrix metallopeptidase 4 (MMP-4), matrix metallopeptidase 5 (MMP-5), matrix metallopeptidase 6 (MMP-6), matrix metallopeptidase 15 (MMP-15), neutrophil elastase, protease activated receptor 2 (PAR2), plasmin, prostasin, PSMA-FOLH1, membrane type serine protease 1 (MT-SP1), matriptase, and u-plasminogen; or
ii. said mammalian protease is selected from the group consisting of matrix metallopeptidase 1 (MMP1), matrix metallopeptidase 2 (MMP2), matrix metallopeptidase 7 (MMP7), matrix metallopeptidase 9 (MMP9), matrix metallopeptidase 11 (MMP11), matrix metallopeptidase 14 (MMP14), urokinase-type plasminogen activator (uPA), legumain, and matriptase;
g. said mammalian protease is preferentially expressed or activated in a target tissue or cell; optionally wherein:
i. said target tissue or cell is a tumor;
ii. said target tissue or cell produces or is co-localized with said mammalian protease;
iii. said target tissue or cell contains therein or thereon, or is associated with in proximity thereto, a reporter polypeptide, optionally wherein said reporter polypeptide is a polypeptide selected from the group consisting of coagulation factor, complement component, tubulin, immunoglobulin, apolipoprotein, serum amyloid, insulin, growth factor, fibrinogen, PDZ domain protein, LIM domain protein, c-reactive protein, serum albumin, versican, collagen, elastin, keratin, kininogen-1, alpha-2-antiplasmin, clusterin, biglycan, alpha-1-antitrypsin, transthyretin, alpha-1-antichymotrypsin, glucagon, hepcidin, thymosin beta-4, haptoglobin, hemoglobin subunit alpha, caveolae-associated protein 2, alpha-2-HS-glycoprotein, chromogranin-A, vitronectin, hemopexin, epididymis secretory sperm binding protein, secretogranin-2, angiotensinogen, transgelin-2, pancreatic prohormone, neurosecretory protein VGF, ceruloplasmin, PDZ and LIM domain protein 1, multimerin-1, inter-alpha-trypsin inhibitor heavy chain H2, N-acetylmuramoyl-L-alanine amidase, histone H1.4, adhesion G-protein coupled receptor G6, mannan-binding lectin serine protease 2, prothrombin, deleted in malignant brain tumors 1 protein, desmoglein-3, calsyntenin-1, alpha-2-macroglobulin, myosin-9, sodium/potassium-transporting ATPase subunit gamma, oncoprotein-induced transcript 3 protein, serglycin, histidine-rich glycoprotein, inter-alpha-trypsin inhibitor heavy chain H5, integrin alpha-IIb, membrane-associated progesterone receptor component 1, histone H1.2, rho GDP-dissociation inhibitor 2, zinc-alpha-2-glycoprotein, talin-1, secretogranin-1, neutrophil defensin 3, cytochrome P450 2E1, gastric inhibitory polypeptide, transcription initiation factor TFIID subunit 1, integral membrane protein 2B, pigment epithelium-derived factor, voltage-dependent N-type calcium channel subunit alpha-1B, ras GTPase-activating protein nGAP, type I cytoskeletal 17, sulfhydryl oxidase 1, homeobox protein Hox-B2, transcription factor SOX-10, E3 ubiquitin-protein ligase SIAH2, decorin, secreted protein acidic and rich in cysteine (SPARC), laminin gamma 1 chain, vimentin, and nidogen-1 (NID1), or versican, type II collagen alpha-1 chain, kininogen-1, complement C4-A, complement C4-B, complement C3, alpha-2-antiplasmin, clusterin, biglycan, elastin, fibrinogen alpha chain, alpha-1-antitrypsin, fibrinogen beta chain, type III collagen alpha-1 chain, serum amyloid A-1 protein, transthyretin, apolipoprotein A-I, apolipoprotein A-I Isoform 1, alpha-1-antichymotrypsin, glucagon, hepcidin, serum amyloid A-2 protein, thymosin beta-4, haptoglobin, hemoglobin subunit alpha, caveolae-associated protein 2, alpha-2-HS-glycoprotein, chromogranin-A, vitronectin, hemopexin, epididymis secretory sperm binding protein, zyxin, apolipoprotein secretogranin-2, angiotensinogen, c-reactive protein, serum albumin, transgelin-2, pancreatic prohormone, neurosecretory protein VGF, ceruloplasmin, PDZ and LIM domain protein 1, tubulin alpha-4A chain, multimerin-1, inter-alpha-trypsin inhibitor heavy chain H2, apolipoprotein C-I, fibrinogen gamma chain, N-acetylmuramoyl-L-alanine amidase, immunoglobulin lambda variable 3-21, histone H1.4, adhesion G-protein coupled receptor G6, immunoglobulin lambda variable 3-25, immunoglobulin lambda variable 1-51, immunoglobulin lambda variable 1-36, mannan-binding lectin serine protease 2, immunoglobulin kappa variable 3-20, immunoglobulin kappa variable 2-30, insulin-like growth factor II, apolipoprotein A-II, probable non-functional immunoglobulin kappa variable 2D-24, prothrombin, coagulation factor IX, apolipoprotein L1, deleted in malignant brain tumors 1 protein, desmoglein-3, calsyntenin-1, immunoglobulin lambda constant 3, complement C5, alpha-2-macroglobulin, myosin-9, sodium/potassium-transporting ATPase subunit gamma, immunoglobulin kappa variable 2-28, oncoprotein-induced transcript 3 protein, serglycin, coagulation factor XII, coagulation factor XIII A chain, insulin, histidine-rich glycoprotein, immunoglobulin kappa variable 3-11, immunoglobulin kappa variable 1-39, collagen alpha-1(I) chain, inter-alpha-trypsin inhibitor heavy chain H5, latent-transforming growth factor beta-binding protein 2, integrin alpha-IIb, membrane-associated progesterone receptor component 1, immunoglobulin lambda variable 6-57, immunoglobulin kappa variable 3-15, complement C1r subcomponent-like protein, histone H1.2, rho GDP-dissociation inhibitor 2, latent-transforming growth factor beta-binding protein 4, collagen alpha-1(XVIII) chain, immunoglobulin lambda variable 2-18, zinc-alpha-2-glycoprotein, talin-1, secretogranin-1, neutrophil defensin 3, cytochrome P450 2E1, gastric inhibitory polypeptide, immunoglobulin heavy variable 3-15, immunoglobulin lambda variable 2-11, transcription initiation factor TFIID subunit 1, collagen alpha-1(VII) chain, integral membrane protein 2B, pigment epithelium-derived factor, voltage-dependent N-type calcium channel subunit alpha-1B, immunoglobulin lambda variable 3-27, ras GTPase-activating protein nGAP, keratin, type I cytoskeletal 17, tubulin beta chain, sulfhydryl oxidase 1, immunoglobulin kappa variable 4-1, complement C1r subcomponent, homeobox protein Hox-B2, transcription factor SOX-10, E3 ubiquitin-protein ligase SIAH2, decorin, SPARC, type I collagen alpha-1 chain, type IV collagen alpha-1 chain, laminin gamma 1 chain, vimentin, type III collagen, type IV collagen alpha-3 chain, type VII collagen alpha-1 chain, type VI collagen alpha-1 chain, type V collagen alpha-1 chain, nidogen-1, and type VI collagen alpha-3 chain.

42-46. (canceled)

47. The method of claim 41, wherein said peptide substrate sequence susceptible to cleavage by said mammalian protease is susceptible to cleavage by a plurality of mammalian proteases comprising said mammalian protease; optionally wherein:
a. said peptide substrate sequence susceptible to cleavage by said plurality of mammalian proteases has at most three amino acid substitutions, at most two amino acid substitutions, or at most one amino acid substitution with respect to a sequence set forth in Table 1(j), wherein none of said amino acid substitution is at a position corresponding to an amino acid residue immediately adjacent to a corresponding scissile bond; or
b. said peptide substrate sequence susceptible to cleavage by said plurality of mammalian proteases comprises a sequence set forth in Table 1(j).

48-51. (canceled)

52. The method of claim 40, wherein a portion of said peptide substrate sequence:
a. a portion of said peptide substrate sequence that is N-terminal of said scissile bond has at most three amino acid substitutions, at most two amino acid substitutions, or at most one amino acid substitution with respect to a C-terminal end sequence containing from four to ten amino acid residues of a sequence set forth in Column IV or V of Table A, wherein none of said amino acid substitution is at a position corresponding to an amino acid residue immediately adjacent to a corresponding scissile bond; optionally wherein said portion of said peptide substrate sequence that is N-terminal of said scissile bond comprises a C-terminal end sequence containing from four to ten amino acid residues of a sequence set forth in Column IV or V of Table A;
b. a portion of said peptide substrate sequence that is C-terminal of said scissile bond has at most three amino acid substitutions, at most two amino acid substitutions, or at most one amino acid substitution with respect to an N-terminal end sequence containing from four to ten amino acid residues of a sequence set forth in Column V or VI of Table A, wherein none of said amino acid substitution is at a position corresponding to an amino acid residue immediately adjacent to a corresponding scissile bond, optionally wherein said portion of said peptide substrate sequence that is C-terminal of said scissile bond comprises an N-terminal end sequence containing from four to ten amino acid residues of a sequence set forth in Column V or VI of Table A;
c. said likelihood of said response is determined by said method.

53-56. (canceled)

57. A method for treating a subject in need of a therapeutic agent that is activatable by a mammalian protease expressed in said subject, the method comprising:
administering an effective amount of said therapeutic agent to said subject, wherein said subject has been shown to express in a biological sample from said subject:
(i) a polypeptide comprising at least five or six consecutive amino acid residues shown in a sequence set forth in Column V of Table A; or
(ii) a polypeptide comprising at least five or six consecutive amino acids shown in a sequence set forth in Column IV of Table A; or
(iii) a polypeptide comprising at least five or six consecutive amino acids shown in a sequence set forth in Column VI of Table A; or
(iv) expression level of polypeptide (i), (ii) or (iii) exceeds a threshold.

58. The method of claim 57, wherein:
a. said polypeptide sequence of (i) comprises at least seven, at least eight, at least nine, or at least ten consecutive amino acid residues shown in a sequence set forth in Column V of Table A;
b. said polypeptide of (ii) comprises at least seven, at least eight, at least nine, or at least ten consecutive amino acids shown in a sequence set forth in Column IV of Table A;
c. said polypeptide of (iii) comprises at least seven, at least eight, at least nine, or at least ten consecutive amino acids shown in a sequence set forth in Column VI of Table A;
d. said subject has been shown to express in said biological sample any two of (i)-(iii);
e. said therapeutic agent comprises a peptide substance sequence susceptible to cleavage by said mammalian protease;
f. said threshold is zero or nominal; and/or
g. said subject is determined to have a likelihood of a response to a therapeutic agent.

59-62. (canceled)

63. The method of claim 58, wherein:
a. said peptide substrate sequence is susceptible to cleavage by said mammalian protease at a scissile bond, and wherein said polypeptide of (i), (ii), or (iii) comprises a portion containing at least four consecutive amino acid residues of said peptide substrate sequence that is either N-terminal or C-terminal of said scissile bond; optionally wherein
i. a portion of said peptide substrate sequence that is N-terminal of said scissile bond has at most three amino acid substitutions, at most two amino acid substitutions, or at most one amino acid substitution with respect to a C-terminal end sequence containing from four to ten amino acid residues of a sequence set forth in Column IV or V of Table A, wherein none of said amino acid substitution is at a position corresponding to an amino acid residue immediately adjacent to a corresponding scissile bond;
ii. said portion of said peptide substrate sequence that is N-terminal of said scissile bond comprises a C-terminal end sequence containing from four to ten amino acid residues of a sequence set forth in Column IV or V of Table A;
b. a portion of said peptide substrate sequence that is C-terminal of said scissile bond has at most three amino acid substitutions, at most two amino acid substitutions, or at most one amino acid substitution with respect to an N-terminal end sequence containing from four to ten amino acid residues of a sequence set forth in Column V or VI of Table A, wherein none of said amino acid substitution is at a position corresponding to an amino acid residue immediately adjacent to a corresponding scissile bond;
c. said portion of said peptide substrate sequence that is C-terminal of said scissile bond comprises an N-terminal end sequence containing from four to ten amino acid residues of a sequence set forth in Column V or VI of Table A;
d. a portion of said peptide substrate sequence that is C-terminal of said scissile bond has at most three amino acid substitutions, at most two amino acid substitutions, or at most one amino acid substitution with respect to an N-terminal end sequence containing from four to ten amino acid residues of a sequence set forth in Column V or VI of Table A, wherein none of said amino acid substitution is at a position corresponding to an amino acid residue immediately adjacent to a corresponding scissile bond;
e. said portion of said peptide substrate sequence that is C-terminal of said scissile bond comprises an N-terminal end sequence containing from four to ten amino acid residues of a sequence set forth in Column V or VI of Table A.

64-69. (canceled)

70. The method of claim 40, wherein:
a. said mammalian protease is a serine protease, a cysteine protease, an aspartate protease, a threonine protease, or a metalloproteinase; optionally wherein:
i. said mammalian protease is selected from the group consisting of disintegrin and metalloproteinase domain-containing protein 10 (ADAM10), disintegrin and metalloproteinase domain-containing protein 12 (ADAM12), disintegrin and metalloproteinase domain-containing protein 15 (ADAM15), disintegrin and metalloproteinase domain-containing protein 17 (ADAM17), disintegrin and metalloproteinase domain-containing protein 9 (ADAM9), disintegrin and metalloproteinase with thrombospondin motifs 5 (ADAMTS5), Cathepsin B, Cathepsin D, Cathepsin E, Cathepsin K, cathepsin L, cathepsin S, Fibroblast activation protein alpha, Hepsin, kallikrein-2, kallikrein-4, kallikrein-3, Prostate-specific antigen (PSA), kallikrein-13, Legumain, matrix metallopeptidase 1 (MMP-1), matrix metallopeptidase 10 (MMP-10), matrix metallopeptidase 11 (MMP-11), matrix metallopeptidase 12 (MMP-12), matrix metallopeptidase 13 (MMP-13), matrix metallopeptidase 14 (MMP-14), matrix metallopeptidase 16 (MMP-16), matrix metallopeptidase 2 (MMP-2), matrix metallopeptidase 3 (MMP-3), matrix metallopeptidase 7 (MMP-7), matrix metallopeptidase 8 (MMP-8), matrix metallopeptidase 9 (MMP-9), matrix metallopeptidase 4 (MMP-4), matrix metallopeptidase 5 (MMP-5), matrix metallopeptidase 6 (MMP-6), matrix metallopeptidase 15 (MMP-15), neutrophil elastase, protease activated receptor 2 (PAR2), plasmin, prostasin, PSMA-FOLH1, membrane type serine protease 1 (MT-SP1), matriptase, and u-plasminogen; or
ii. said mammalian protease is selected from the group consisting of matrix metallopeptidase 1 (MMP1), matrix metallopeptidase 2 (MMP2), matrix metallopeptidase 7 (MMP7), matrix metallopeptidase 9 (MMP9), matrix metallopeptidase 11 (MMP11), matrix metallopeptidase 14 (MMP14), urokinase-type plasminogen activator (uPA), legumain, and matriptase;
b. said mammalian protease is preferentially expressed or activated in a target tissue or cell; optionally wherein:
i. said target tissue or cell is a tumor;
ii. said target tissue or cell produces or is co-localized with said mammalian protease;
iii. said target tissue or cell contains therein or thereon, or is associated with in proximity thereto, a reporter polypeptide; optionally wherein: said reporter polypeptide is a polypeptide selected from the group consisting of coagulation factor, complement component, tubulin, immunoglobulin, apolipoprotein, serum amyloid, insulin, growth factor, fibrinogen, PDZ domain protein, LIM domain protein, c-reactive protein, serum albumin, versican, collagen, elastin, keratin, kininogen-1, alpha-2-antiplasmin, clusterin, biglycan, alpha-1-antitrypsin, transthyretin, alpha-1-antichymotrypsin, glucagon, hepcidin, thymosin beta-4, haptoglobin, hemoglobin subunit alpha, caveolae-associated protein 2, alpha-2-HS-glycoprotein, chromogranin-A, vitronectin, hemopexin, epididymis secretory sperm binding protein, secretogranin-2, angiotensinogen, transgelin-2, pancreatic prohormone, neurosecretory protein VGF, ceruloplasmin, PDZ and LIM domain protein 1, multimerin-1, inter-alpha-trypsin inhibitor heavy chain H2, N-acetylmuramoyl-L-alanine amidase, histone H1.4, adhesion G-protein coupled receptor G6, mannan-binding lectin serine protease 2, prothrombin, deleted in malignant brain tumors 1 protein, desmoglein-3, calsyntenin-1, alpha-2-macroglobulin, myosin-9, sodium/potassium-transporting ATPase subunit gamma, oncoprotein-induced transcript 3 protein, serglycin, histidine-rich glycoprotein, inter-alpha-trypsin inhibitor heavy chain H5, integrin alpha-IIb, membrane-associated progesterone receptor component 1, histone H1.2, rho GDP-dissociation inhibitor 2, zinc-alpha-2-glycoprotein, talin-1, secretogranin-1, neutrophil defensin 3, cytochrome P450 2E1, gastric inhibitory polypeptide, transcription initiation factor TFIID subunit 1, integral membrane protein 2B, pigment epithelium-derived factor, voltage-dependent N-type calcium channel subunit alpha-1B, ras GTPase-activating protein nGAP, type I cytoskeletal 17, sulfhydryl oxidase 1, homeobox protein Hox-B2, transcription factor SOX-10, E3 ubiquitin-protein ligase SIAH2, decorin, secreted protein acidic and rich in cysteine (SPARC), laminin gamma 1 chain, vimentin, and nidogen-1 (NID1) or said reporter polypeptide is a polypeptide selected from the group consisting of versican, type II collagen alpha-1 chain, kininogen-1, complement C4-A, complement C4-B, complement C3, alpha-2-antiplasmin, clusterin, biglycan, elastin, fibrinogen alpha chain, alpha-1-antitrypsin, fibrinogen beta chain, type III collagen alpha-1 chain, serum amyloid A-1 protein, transthyretin, apolipoprotein A-I, apolipoprotein A-I Isoform 1, alpha-1-antichymotrypsin, glucagon, hepcidin, serum amyloid A-2 protein, thymosin beta-4, haptoglobin, hemoglobin subunit alpha, caveolae-associated protein 2, alpha-2-HS-glycoprotein, chromogranin-A, vitronectin, hemopexin, epididymis secretory sperm binding protein, zyxin, apolipoprotein secretogranin-2, angiotensinogen, c-reactive protein, serum albumin, transgelin-2, pancreatic prohormone, neurosecretory protein VGF, ceruloplasmin, PDZ and LIM domain protein 1, tubulin alpha-4A chain, multimerin-1, inter-alpha-trypsin inhibitor heavy chain H2, apolipoprotein C-I, fibrinogen gamma chain, N-acetylmuramoyl-L-alanine amidase, immunoglobulin lambda variable 3-21, histone H1.4, adhesion G-protein coupled receptor G6, immunoglobulin lambda variable 3-25, immunoglobulin lambda variable 1-51, immunoglobulin lambda variable 1-36, mannan-binding lectin serine protease 2, immunoglobulin kappa variable 3-20, immunoglobulin kappa variable 2-30, insulin-like growth factor II, apolipoprotein A-II, probable non-functional immunoglobulin kappa variable 2D-24, prothrombin, coagulation factor IX, apolipoprotein L1, deleted in malignant brain tumors 1 protein, desmoglein-3, calsyntenin-1, immunoglobulin lambda constant 3, complement C5, alpha-2-macroglobulin, myosin-9, sodium/potassium-transporting ATPase subunit gamma, immunoglobulin kappa variable 2-28, oncoprotein-induced transcript 3 protein, serglycin, coagulation factor XII, coagulation factor XIII A chain, insulin, histidine-rich glycoprotein, immunoglobulin kappa variable 3-11, immunoglobulin kappa variable 1-39, collagen alpha-1(I) chain, inter-alpha-trypsin inhibitor heavy chain H5, latent-transforming growth factor beta-binding protein 2, integrin alpha-IIb, membrane-associated progesterone receptor component 1, immunoglobulin lambda variable 6-57, immunoglobulin kappa variable 3-15, complement C1r subcomponent-like protein, histone H1.2, rho GDP-dissociation inhibitor 2, latent-transforming growth factor beta-binding protein 4, collagen alpha-1(XVIII) chain, immunoglobulin lambda variable 2-18, zinc-alpha-2-glycoprotein, talin-1, secretogranin-1, neutrophil defensin 3, cytochrome P450 2E1, gastric inhibitory polypeptide, immunoglobulin heavy variable 3-15, immunoglobulin lambda variable 2-11, transcription initiation factor TFIID subunit 1, collagen alpha-1(VII) chain, integral membrane protein 2B, pigment epithelium-derived factor, voltage-dependent N-type calcium channel subunit alpha-1B, immunoglobulin lambda variable 3-27, ras GTPase-activating protein nGAP, keratin, type I cytoskeletal 17, tubulin beta chain, sulfhydryl oxidase 1, immunoglobulin kappa variable 4-1, complement C1r subcomponent, homeobox protein Hox-B2, transcription factor SOX-10, E3 ubiquitin-protein ligase SIAH2, decorin, SPARC, type I collagen alpha-1 chain, type IV collagen alpha-1 chain, laminin gamma 1 chain, vimentin, type III collagen, type IV collagen alpha-3 chain, type VII collagen alpha-1 chain, type VI collagen alpha-1 chain, type V collagen alpha-1 chain, nidogen-1, and type VI collagen alpha-3 chain;
iv. said reporter polypeptide comprises a sequence set forth in Columns II-VI of Table A;
c. said target tissue or cell is characterized by an increased amount or activity of said mammalian protease in proximity to said target tissue or cell as compared to a non-target tissue or cell in said subject.

71-80. (canceled)

81. The method of claim 40, wherein said subject is suffering from, or is suspected of suffering from, a disease or condition characterized by an increased expression or activity of said mammalian protease in proximity to a target tissue or cell as compared to a corresponding non-target tissue or cell in said subject optionally wherein said disease or condition is a cancer or an inflammatory or autoimmune disease; optionally wherein said disease or condition is selected from:
a. the group consisting of ankylosing spondylitis (AS), arthritis (for example, and not limited to, rheumatoid arthritis (RA), juvenile idiopathic arthritis (JIA), osteoarthritis (OA), psoriatic arthritis (PsA), gout, chronic arthritis), chagas disease, chronic obstructive pulmonary disease (COPD), dermatomyositis, type 1 diabetes, endometriosis, Goodpasture syndrome, Graves' disease, Guillain-Barre syndrome (GBS), Hashimoto's disease, suppurative scab, Kawasaki disease, IgA nephropathy, idiopathic thrombocytopenic purpura, inflammatory bowel disease (IBD) (for example, and not limited to, Crohn's disease (CD), clonal disease, ulcerative colitis, collagen colitis, lymphocytic colitis, ischemic colitis, empty colitis, Behcet's syndrome, infectious colitis, indeterminate colitis, interstitial Cystitis), lupus (for example, and not limited to, systemic lupus erythematosus, discoid lupus, subacute cutaneous lupus erythematosus, cutaneous lupus erythematosus (such as chilblain lupus erythematosus), drug-induced lupus, neonatal lupus, lupus nephritis), mixed connective tissue disease, morphea, multiple sclerosis (MS), severe muscle Force disorder, narcolepsy, neuromuscular angina, pemphigus vulgaris, pernicious anemia, psoriasis, psoriatic arthritis, polymyositis, primary biliary cirrhosis, relapsing polychondritis, schizophrenia, scleroderma, Sjogren's syndrome, systemic stiffness syndrome, temporal arteritis (also known as giant cell arteritis), vasculitis, vitiligo, Wegener's granulomatosis, transplant rejection-associated immune reaction(s) (for example, and not limited to, renal transplant rejection, lung transplant rejection, liver transplant rejection), psoriasis, Wiskott-Aldrich syndrome, autoimmune lymphoproliferative syndrome, myasthenia gravis, inflammatory chronic rhinosinusitis, colitis, celiac disease, Barrett's esophagus, inflammatory gastritis, autoimmune nephritis, autoimmune hepatitis, autoimmune carditis, autoimmune encephalitis, autoimmune mediated hematological disease, asthma, atopic dermatitis, atopy, allergy, allergic rhinitis, scleroderma, bronchitis, pericarditis, the inflammatory disease is, Alzheimer's disease, Parkinson's disease, amyotrophic lateral sclerosis, inflammatory lung disease, inflammatory skin disease, atherosclerosis, myocardial infarction, stroke, gram-positive shock, gram-negative shock, sepsis, septic shock, hemorrhagic shock, anaphylactic shock, systemic inflammatory response syndrome; or
b. the group consisting of carcinoma, Hodgkin's lymphoma, and non-Hodgkin's lymphoma, diffuse large B cell lymphoma, follicular lymphoma, mantle cell lymphoma, blastoma, breast cancer, ER/PR+ breast cancer, Her2+ breast cancer, triple-negative breast cancer, colon cancer, colon cancer with malignant ascites, mucinous tumors, prostate cancer, head and neck cancer, skin cancer, melanoma, genito-urinary tract cancer, ovarian cancer, ovarian cancer with malignant ascites, peritoneal carcinomatosis, uterine serous carcinoma, endometrial cancer, cervix cancer, colorectal, uterine cancer, mesothelioma in the peritoneum, kidney cancer, Wilm's tumor, lung cancer, small-cell lung cancer, non-small cell lung cancer, gastric cancer, stomach cancer, small intestine cancer, liver cancer, hepatocarcinoma, hepatoblastoma, liposarcoma, pancreatic cancer, gall bladder cancer, cancers of the bile duct, esophageal cancer, salivary gland carcinoma, thyroid cancer, epithelial cancer, arrhenoblastoma, adenocarcinoma, sarcoma, and B-cell derived chronic lymphatic leukemia.

82-84. (canceled)

85. The method of claim 40, wherein:
a. said therapeutic agent is an anti-cancer agent;
b. said therapeutic agent is an activatable therapeutic agent optionally wherein said therapeutic agent is a non-natural, activatable therapeutic agent;
c. said therapeutic agent comprises a masking moiety (MM); optionally wherein:
i. said masking moiety (MM) is capable of being released from said therapeutic agent upon cleavage of said peptide substrate sequence by said mammalian protease;
ii. said masking moiety (MM) interferes with an interaction of said therapeutic agent, in an uncleaved state, to a target tissue or cell;
iii. said bioactivity of said therapeutic agent is capable of being enhanced upon cleavage of said peptide substrate sequence by said mammalian protease;
iv. said masking moiety is an extended recombinant polypeptide; optionally wherein the extended recombinant polypeptide is characterized in that (i) it comprises at least 100 amino acids; (ii) at least 90% of the amino acid residues of it are selected from glycine (G), alanine (A), serine (S), threonine (T), glutamate (E) and proline (P); and (iii) it comprises at least 4 different types of amino acids selected from G, A, S, T, E, and P.

86-94. (canceled)

95. A method for treating a disease or condition in a subject, comprising administering to said subject in need thereof one or more therapeutically effective doses of a therapeutic agent or a pharmaceutical composition.

96. The method of claim 95, wherein:
a. said subject is selected from the group consisting of mouse, rat, monkey, and human, optionally wherein said subject is a human;
b. said subject is determined to have a likelihood of a response to said therapeutic agent or said pharmaceutical composition; optionally wherein:
i. said likelihood of said response is 50% or higher; and/or
ii. said likelihood of said response is determined by said method;
c. said disease or condition is a cancer or an inflammatory or autoimmune disease; optionally wherein said disease or condition is selected from:
i. the group consisting of ankylosing spondylitis (AS), arthritis (for example, and not limited to, rheumatoid arthritis (RA), juvenile idiopathic arthritis (JIA), osteoarthritis (OA), psoriatic arthritis (PsA), gout, chronic arthritis), chagas disease, chronic obstructive pulmonary disease (COPD), dermatomyositis, type 1 diabetes, endometriosis, Goodpasture syndrome, Graves' disease, Guillain-Barre syndrome (GBS), Hashimoto's disease, suppurative scab, Kawasaki disease, IgA nephropathy, idiopathic thrombocytopenic purpura, inflammatory bowel disease (IBD) (for example, and not limited to, Crohn's disease (CD), clonal disease, ulcerative colitis, collagen colitis, lymphocytic colitis, ischemic colitis, empty colitis, Behcet's syndrome, infectious colitis, indeterminate colitis, interstitial Cystitis), lupus (for example, and not limited to, systemic lupus erythematosus, discoid lupus, subacute cutaneous lupus erythematosus, cutaneous lupus erythematosus (such as chilblain lupus erythematosus), drug-induced lupus, neonatal lupus, lupus nephritis), mixed connective tissue disease, morphea, multiple sclerosis (MS), severe muscle Force disorder, narcolepsy, neuromuscular angina, pemphigus vulgaris, pernicious anemia, psoriasis, psoriatic arthritis, polymyositis, primary biliary cirrhosis, relapsing polychondritis, schizophrenia, scleroderma, Sjogren's syndrome, systemic stiffness syndrome, temporal arteritis (also known as giant cell arteritis), vasculitis, vitiligo, Wegener's granulomatosis, transplant rejection-associated immune reaction(s) (for example, and not limited to, renal transplant rejection, lung transplant rejection, liver transplant rejection), psoriasis, Wiskott-Aldrich syndrome, autoimmune lymphoproliferative syndrome, myasthenia gravis, inflammatory chronic rhinosinusitis, colitis, celiac disease, Barrett's esophagus, inflammatory gastritis, autoimmune nephritis, autoimmune hepatitis, autoimmune carditis, autoimmune encephalitis, autoimmune mediated hematological disease, asthma, atopic dermatitis, atopy, allergy, allergic rhinitis, scleroderma, bronchitis, pericarditis, the inflammatory disease is, Alzheimer's disease, Parkinson's disease, amyotrophic lateral sclerosis, inflammatory lung disease, inflammatory skin disease, atherosclerosis, myocardial infarction, stroke, gram-positive shock, gram-negative shock, sepsis, septic shock, hemorrhagic shock, anaphylactic shock, systemic inflammatory response syndrome; or
ii. the group consisting of carcinoma, Hodgkin's lymphoma, and non-Hodgkin's lymphoma, diffuse large B cell lymphoma, follicular lymphoma, mantle cell lymphoma, blastoma, breast cancer, ER/PR+ breast cancer, Her2+ breast cancer, triple-negative breast cancer, colon cancer, colon cancer with malignant ascites, mucinous tumors, prostate cancer, head and neck cancer, skin cancer, melanoma, genito-urinary tract cancer, ovarian cancer, ovarian cancer with malignant ascites, peritoneal carcinomatosis, uterine serous carcinoma, endometrial cancer, cervix cancer, colorectal, uterine cancer, mesothelioma in the peritoneum, kidney cancer, Wilm's tumor, lung cancer, small-cell lung cancer, non-small cell lung cancer, gastric cancer, stomach cancer, small intestine cancer, liver cancer, hepatocarcinoma, hepatoblastoma, liposarcoma, pancreatic cancer, gall bladder cancer, cancers of the bile duct, esophageal cancer, salivary gland carcinoma, thyroid cancer, epithelial cancer, arrhenoblastoma, adenocarcinoma, sarcoma, and B-cell derived chronic lymphatic leukemia.

97-105. (canceled)

106. A kit for the practice of a method of claim 1 for assessing a likelihood of a subject being responsive to a therapeutic agent that is activatable by a mammalian protease expressed in said subject having a disease or disorder comprising a reagent for detecting the presence or amount of a proteolytic peptide product produced by action of said mammalian protease.
